(19) United States
(12) Patent Application Publication
(10) Pub. No.: US 2024/0246954 A1
(43) Pub. Date: Jul. 25, 2024

(54) COMPOSITIONS AND METHODS FOR INHIBITING KRAS

(71) Applicants: TheRas, Inc., Palo Alto, CA (US); Leidos Biomedical Research, Inc., Frederick, MD (US); Lawrence Livermore National Security, LLC, Livermore, CA (US)

(72) Inventors: Bin Wang, Dallas, TX (US); Rui Xu, Dallas, TX (US); Eli Wallace, Loveland, CO (US); Felice Lightstone, Fremont, CA (US); Yue Yang, Foster City, CA (US); Paola Bisignano, San Francisco, CA (US); Anna Elzbieta Maciag, Frederick, MD (US); David Michael Turner, Frederick, MD (US); Dhirendra Kumar Simanshu, Frederick, MD (US); Albert Hay Wah Chan, Frederick, MD (US); Zuhui Zhang, Millbrae, CA (US); Christopher John Brassard, Frederick, MD (US); Tao Liao, Frederick, MD (US)

(21) Appl. No.: 18/546,317

(22) PCT Filed: Feb. 15, 2022
(86) PCT No.: PCT/US22/16487

§ 371 Date: Aug. 14, 2023

Related U.S. Application Data
(60) Provisional application No. 63/246,181, filed on Sep. 20, 2021.
(60) Provisional application No. 63/150,011, filed on Feb. 16, 2021.

(51) Int. Cl. C07D 417/14; A61K 31/517; A61K 31/519; A61K 31/5377; A61K 31/551; C07D 403/12; C07D 413/14; C07D 417/04; C07D 471/04; C07D 487/04; C07D 487/08; C07D 487/10; C07D 519/00

(52) U.S. Cl. CPC C07D 417/14; A61K 31/517; A61K 31/519; A61K 31/5377; A61K 31/551; C07D 403/12; C07D 413/14; C07D 417/04; C07D 471/04; C07D 487/04; C07D 487/08; C07D 487/10; C07D 519/00

(57) ABSTRACT

Provided herein are compounds of formula (I) and (II), or salts, esters, tautomers, prodrugs, zwitterionic forms, or stereoisomers thereof, as well as pharmaceutical compositions comprising the same. Also provided herein are methods of using the same in modulating (e.g., inhibiting) KRAS (e.g., KRAS having a G12D mutation) and treating diseases or disorders such as cancers in subjects in need thereof.

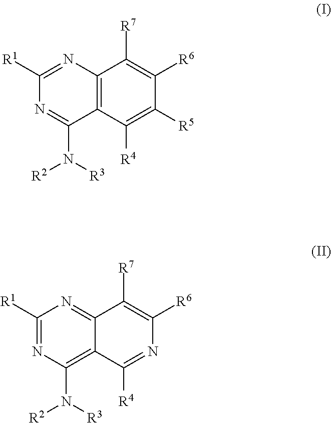

### RELATED APPLICATIONS

This application claims priority to and benefit of U.S. Provisional Application No. 63/150,011, filed Feb. 16, 2021, and U.S. Provisional Application No. 63/246,181, filed Sep. 20, 2021, the entire contents of each of which are hereby incorporated by reference.

### STATEMENT AS TO RIGHTS TO INVENTIONS MADE UNDER FEDERALLY SPONSORED RESEARCH AND DEVELOPMENT

This invention was made with government support under (1) Contract No.: 75N91019D00024 awarded by the National Institutes of Health and (2) Contract No. DE-AC52-07NA27344 awarded by the United States Department of Energy. The government has certain rights in the invention.

### BACKGROUND

RAS mutations occur in approximately 20-30% of human cancers, including the majority of pancreatic ductal adenocarcinoma (PDAC), half of colorectal cancers, and a third of all lung cancers. With the highest RAS mutation frequencies seen with the top three causes of cancer deaths in the United States (lung, colorectal, and pancreatic cancer), the development of anti-RAS therapies is a major priority and a major challenge for cancer research. RAS proteins did not appear to present suitable pockets to which drugs could bind, except for the GDP/GTP binding site. Unfortunately, RAS proteins bind to these nucleotides with very high (picomolar) affinities, making the development of effective nucleotide analogs virtually impossible. Attempts to block pathways downstream of RAS with a hope to provide clinical benefit for patients suffering from RAS-driven cancers have been generally disappointing. Very recently, allele-specific covalent KRAS G12C inhibitors entered clinical trials, and early clinical data show some effectiveness, at least in lung cancer.

The three RAS genes (HRAS, NRAS, and KRAS) encode four 188-189 amino acid proteins that share 82%-90% amino acid sequence identity and near-identical structural and biochemical properties. However, they are differentially expressed, and mutated with different frequencies in cancer. KRAS is the most frequently mutated oncogene in cancer and KRAS mutation is commonly associated with poor prognosis and resistance to therapy. Significant cancer type preferences exist among the RAS genes. KRAS mutations predominate in lung, colorectal, and pancreatic cancer, whereas NRAS mutations predominate in cutaneous melanomas and acute myelogenous leukemia, and HRAS mutations are found in bladder and head and neck squamous cell carcinomas.

KRAS is mutationally activated in 94% of pancreatic cancers. Pancreatic cancer (e.g., pancreatic ductal adenocarcinoma (PDAC)) became the third leading cause of cancer deaths in the United States in 2016. With a continued increase in incidence, pancreatic cancer is projected to become the second leading cause of cancer death by 2020. With no biomarkers for early detection, late onset of symptoms when the cancer has already reached the metastatic state, and the 5-year survival rate of 8%, pancreatic cancer is the deadliest cancer in the US.

KRAS mutations are the initiating genetic step in pancreatic cancer; however, continued mutant KRAS function is required to maintain the growth of PDAC. RNA interference-mediated KRAS inactivation in KRAS G12D-driven PDAC showed rapid regression of tumor growth. These data support the significance of mutant KRAS as a therapeutic target in PDAC. Since 40% of PDACs are driven by KRAS G12D mutant, inhibitors targeting this mutation are highly desirable. KRAS G12D mutation also occurs in high frequency in lung and colorectal cancers, making KRAS G12D desirable therapeutic target for direct G12D allele-specific inhibitors.

### SUMMARY

In an aspect, the present disclosure provides compositions comprising compounds represented by Formula I or Formula II:

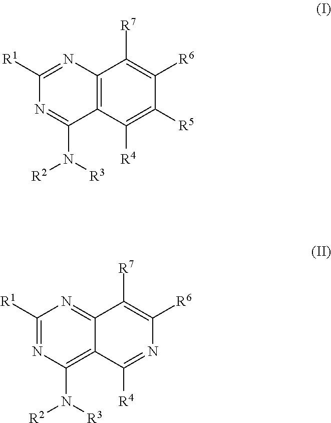

or a salt, ester, tautomer, prodrug, zwitterionic form, or stereoisomer thereof, wherein R¹, R², R³, R⁴, R⁵, R⁶, and R⁷ are as provided herein. In some embodiments, a compound provided herein, or a salt, ester, tautomer, zwitterionic form, or stereoisomer thereof, can modulate the activity of a KRAS protein, such as a KRAS protein having a G12D mutation.

In another aspect, the present disclosure provides a pharmaceutical composition comprising a compound represented by Formula I or Formula II, together with a pharmaceutically acceptable carrier.

In a further aspect, the present disclosure provides a method of inhibition of KRAS activity in a human or animal subject for the treatment of a disease such as cancer, including pancreatic cancer (e.g., pancreatic ductal adenocarcinoma (PDAC)), colorectal cancer, and lung cancer.

In another aspect, the present disclosure provides a use of a compound provided herein, or a salt, ester, tautomer, zwitterionic form, or stereoisomer thereof, in the manufacture of a medicament for the treatment of a disease, disorder, or condition (e.g., a cancer) ameliorated, treated, inhibited, or reduced by inhibition of KRAS, including KRAS having a G12D mutation. In some embodiments, the disease, disorder, or condition is pancreatic cancer (e.g., pancreatic ductal adenocarcinoma (PDAC)), colorectal cancer, or lung cancer.

In a further aspect, the present disclosure provides a compound as provided herein, or a salt, ester, tautomer, zwitterionic form, or stereoisomer thereof, for use as a medicament. In some embodiments, the medicament is used in the treatment of a disease, disorder, or condition (e.g., a cancer). In some embodiments, the disease, disorder, or condition is pancreatic cancer (e.g., pancreatic ductal adenocarcinoma (PDAC)), colorectal cancer, or lung cancer.

### DETAILED DESCRIPTION

The present disclosure provides compounds of Formulas I and II, which compounds may possess useful KRAS inhibitory activity, and may be used in the treatment or prophylaxis of a disease, disorder, or condition in which KRAS plays an active role. In particular, certain compounds provided herein may possess useful inhibitory activity of KRAS having a G12D mutation, which KRAS protein is in an active (GTP-bound) or inactive (GDP-bound) conformation. The present disclosure also provides pharmaceutical compositions comprising one or more compounds provided herein together with a pharmaceutically acceptable carrier, as well as methods of making and using the compounds and compositions. The present disclosure also provides methods for inhibiting KRAS, including KRAS having a G12D mutation, which KRAS is in an active or inactive conformation. In an aspect, the present disclosure provides a method for treating a disorder mediated by KRAS including a KRAS having a G12D mutation in a subject in need of such treatment, which method comprises administering to the subject a therapeutically effective amount of a compound or composition provided herein. Also provided herein is the use of certain compounds provided herein in the manufacture of a medicament for the treatment of a disease, disorder, or condition ameliorated, treated, inhibited, or reduced by inhibition of KRAS, including KRAS having a G12D mutation. In some embodiments, the disease, disorder, or condition is a cancer.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

“Alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

“Alkynyl” refers to either a straight chain or branched-chain hydrocarbon having at least 2 carbon atoms and at least one triple bond and having the number of carbon atoms indicated (i.e., C₂₋₆ means to two to six carbons). Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, and 1,3,5-hexatriynyl.

“Alkoxy,” as used herein, alone or in combination, refers to an alkyl ether radical, wherein the term alkyl is as described herein. Examples of suitable alkyl ether radicals include methoxy, ethoxy, n-propoxy, isopropoxy, n-butoxy, iso-butoxy, sec-butoxy, tert-butoxy, and the like.

“Alkyl,” as used herein, alone or in combination, refers to a straight-chain or branched-chain alkyl radical containing from 1 to 20 carbon atoms (e.g., C₁₋₂₀ alkyl). In certain embodiments, said alkyl will comprise from 1 to 10 carbon atoms (e.g., C₁₋₁₀ alkyl). In further embodiments, said alkyl will comprise from 1 to 8 carbon atoms (e.g., C₁₋₈ alkyl). In further embodiments, said alkyl will comprise from 1 to 6 carbon atoms (e.g., C₁₋₆ alkyl). In further embodiments, said alkyl will comprise from 1 to 3 carbon atoms (e.g., C₁₋₃ alkyl). Alkyl groups are unsubstituted or substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

“Alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino, and the like.

“Alkylthio,” as used herein, alone or in combination, refers to an alkyl thioether (R—S—) radical wherein the term alkyl is as described herein and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

“Amido” and “carbamoyl,” as used herein, alone or in combination, refer to an amino group as described herein attached to the parent molecular moiety through a carbonyl group, or vice versa. The “amido” group as used herein incudes a “C-amido” and “N-amido” groups. The term “C-amido” as used herein, alone or in combination, refers to a —C(O)N(RR′) group with R and R′ as defined herein or as defined by the specifically enumerated “R” groups designated. In some embodiments, the “amido” group includes —C(O)NH₂, C₁₋₄alkylamido, and di(C₁₋₄alkyl)amido. The term “C₁₋₄alkylamido”, as used herein, refers to —C(O)NH(C₁₋₄alkyl), wherein C₁₋₄alkyl is as defined herein. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

“Amino,” as used herein, alone or in combination, refers to —NRR′, wherein R and R′ are independently selected from hydrogen, alkyl, acyl, heteroalkyl, aryl, cycloalkyl, heteroaryl, and heterocycloalkyl, any of which may themselves be unsubstituted or substituted. Additionally, R and R′ may combine to form a heterocycloalkyl, which is unsubstituted or substituted. An “amino” group may be a primary amine (e.g., —NH₂), secondary or di-substituted amine (e.g., —NHR where R is not hydrogen), or tertiary or tri-substituted amine (e.g., —NRR′ where neither R nor R′ is hydrogen).

“Aryl,” as used herein, alone or in combination, means a carbocyclic aromatic system containing one, two, or three rings wherein such polycyclic ring systems are fused together. The term “aryl” embraces aromatic groups such as phenyl, naphthyl, anthracenyl, and phenanthryl. An aryl moiety may include, for example, between 5 to 20 carbon atoms, such as between 5 to 12 carbon atoms, such as 5 or 6 carbon atoms.

“Arylalkenyl” or “aralkenyl,” as used herein, alone or in combination, refers to an aryl group attached to the parent molecular moiety through an alkenyl group.

“Arylalkoxy” or “aralkoxy,” as used herein, alone or in combination, refers to an aryl group attached to the parent molecular moiety through an alkoxy group.

“Arylalkyl” or “aralkyl,” as used herein, alone or in combination, refers to an aryl group attached to the parent molecular moiety through an alkyl group.

“Aryloxy,” as used herein, alone or in combination, refers to an aryl group attached to the parent molecular moiety through an oxy.

“Carbamate,” as used herein, alone or in combination, refers to an ester of carbamic acid (—NHCOO—) which may be attached to the parent molecular moiety from either the nitrogen or acid end, and which is unsubstituted or substituted as defined herein.

“O-carbamyl” as used herein, alone or in combination, refers to a —OC(O)NRR′, group, with R and R′ as defined herein.

“N-carbamyl” as used herein, alone or in combination, refers to a ROC(O)NR′— group, with R and R′ as defined herein.

“Carbonyl,” as used herein, when alone includes formyl [—C(O)H] and in combination is a —C(O)— group.

“Carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

“Cyano,” as used herein, alone or in combination, refers to —CN.

“Cycloalkyl,” or, alternatively, “carbocycle,” as used herein, alone or in combination, refers to a saturated or partially saturated monocyclic, bicyclic, or tricyclic alkyl group wherein each cyclic moiety contains from 3 to 12 carbon atom ring members and which may optionally be a benzo fused ring system which is unsubstituted or substituted as defined herein. A carbocycle may comprise a bridged ring system and/or a spiro ring system (e.g., a system including two rings sharing a single carbon atom). The term “cycloalkenyl” refers to a cycloalkyl group having one or two double bonds. In certain embodiments, said cycloalkyl (or cycloalkenyl) will comprise from 5 to 7 carbon atoms. Examples of such groups include cyclopropyl, cyclobutyl, cyclopentyl, cyclohexyl, cycloheptyl, cyclobutenyl, cyclopentenyl, cyclohexenyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene and octahydronaphthalene, as well as the multicyclic (multicentered) saturated or partially unsaturated type. The latter type of isomer is exemplified in general by bicyclo[1,1,1]pentane, camphor, adamantane, and bicyclo[3,2,1]octane.

“Ester,” as used herein, alone or in combination, refers to a carboxy group bridging two moieties linked at carbon atoms.

“Ether,” as used herein, alone or in combination, refers to an oxy group bridging two moieties linked at carbon atoms.

“Halo,” or “halogen,” as used herein, alone or in combination, refers to fluorine, chlorine, bromine, or iodine.

“Haloalkoxy,” as used herein, alone or in combination, refers to a haloalkyl group attached to the parent molecular moiety through an oxygen atom.

“Haloalkyl,” as used herein, alone or in combination, refers to an alkyl radical having the meaning as described herein wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro, or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

“Heteroalkyl,” as used herein, alone or in combination, refers to a stable straight or branched hydrocarbon chain, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

“Heteroaryl,” as used herein, alone or in combination, refers to a 3 to 15 membered aromatic monocyclic ring, or a fused monocyclic, bicyclic, or tricyclic ring system in which at least one of the fused rings is aromatic, which ring or ring system contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, phenanthrolinyl, dibenzofuranyl, acridinyl, phenanthridinyl, xanthenyl and the like.

“Heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. A heterocycle may comprise a bridged ring system and/or a spiro ring system (e.g., a system including two rings sharing a single atom, such as a single carbon atom). “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups are unsubstituted or substituted unless specifically prohibited.

“Hydrazinyl” as used herein, alone or in combination, refers to two amino groups joined by a single bond, i.e., —N—N—.

“Hydroxy,” as used herein, alone or in combination, refers to —OH.

“Hydroxyalkyl,” as used herein, alone or in combination, refers to a hydroxy group attached to the parent molecular moiety through an alkyl group.

“Iminohydroxy,” as used herein, alone or in combination, refers to ═N(OH) and ═N—O—.

“Lower amino,” as used herein, alone or in combination, refers to —NRR′, wherein R and R′ are independently selected from hydrogen and lower alkyl, either of which is unsubstituted or substituted.

“Mercaptyl” as used herein, alone or in combination, refers to an RS— group, where R is as defined herein.

“Nitro,” as used herein, alone or in combination, refers to —NO₂.

“Oxy” or “oxa,” as used herein, alone or in combination, refer to —O—.

“Oxo,” as used herein, alone or in combination, refers to ═O.

“Perhaloalkoxy” refers to an alkoxy group where all of the hydrogen atoms are replaced by halogen atoms.

“Perhaloalkyl” as used herein, alone or in combination, refers to an alkyl group where all of the hydrogen atoms are replaced by halogen atoms.

“Ring,” or equivalently, “cycle,” as used herein, in reference to a chemical structure or portion thereof, means a group in which every atom is a member of a common cyclic structure. A ring can be saturated or unsaturated, including aromatic, unless otherwise provided, and may have between 3 and 9 members. If the ring is a heterocycle, it may contain between 1 and 4 heteroatoms or heteroatom-comprising groups selected from B, N, O, S, C(O), S(O)ₘ. Unless specifically prohibited, a ring is unsubstituted or substituted. Two or more rings may be fused together (e.g., they may share a bond and two common atoms). Two or more rings may be linked together in a spiro arrangement such that only a single atom is shared between two rings. Two or more rings may also or alternatively be configured in a bridged arrangement such that three or more atoms are shared between two or more rings.

“Sulfonate,” “sulfonic acid,” and “sulfonic,” as used herein, alone or in combination, refer to the —SO₃H group and its anion as the sulfonic acid is used in salt formation.

“Sulfanyl,” as used herein, alone or in combination, refers to —S—.

“Sulfinyl,” as used herein, alone or in combination, refers to —S(O)—.

“Sulfonyl,” as used herein, alone or in combination, refers to —S(O)₂—.

“N-sulfonamido” refers to a RS(═O)₂NR′— group with R and R′ as defined herein.

“S-sulfonamido” refers to a —S(═O)₂NRR′, group, with R and R′ as defined herein.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers that are well known in the art include keto/enol, enamine/imine, and lactam/lactim tautomers. Examples of tautomers that are well known in the art also include 2-hydroxypyridine/2(1H)-pyridone and 2-aminopyridine/2(1H)-iminopyridone tautomers.

“Thia” and “thio,” as used herein, alone or in combination, refer to a —S-group or an ether wherein the oxygen is replaced with sulfur. The oxidized derivatives of the thio group, namely sulfinyl and sulfonyl, are included in the definition of thia and thio.

“Thiol,” as used herein, alone or in combination, refers to an —SH group.

“Thiocarbonyl,” as used herein, when alone includes thioformyl —C(S)H and in combination is a —C(S)— group.

“N-thiocarbamyl” refers to an ROC(S)NR′-group, with R and R′ as defined herein.

“O-thiocarbamyl” refers to a —OC(S)NRR′, group with R and R′ as defined herein.

“Thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

As described herein, groups may be substituted or unsubstituted (e.g., “optionally substituted”). Unless otherwise specified, any group may be substituted with one or more substituents, such as one or more substituents provided herein. Examples of substituents that may substitute a group include, but are not limited to, one or more substituents independently selected from the following groups or a particular designated set of groups, alone or in combination: alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), alkynyl (e.g., C₂₋₂₀ alkynyl, such as C₂₋₁₀ alkynyl, such as C₂₋₆ alkynyl), alkanoyl (e.g., C₁₋₂₀ alkanoyl, such as C₁₋₁₀ alkanoyl, such as C₁₋₆ alkanoyl), heteroalkyl (e.g., a heteroalkyl moiety including 1-20 atoms carbon atoms and 1-6 heteroatoms, such as a heteroalkyl moiety including 1-6 carbon atoms and 1-3 heteroatoms), haloalkyl (e.g., a halo-substituted C₁₋₂₀ alkyl, such as a halo-substituted C₁₋₁₀ alkyl, a halo-substituted C₁₋₆ alkyl), haloalkenyl (e.g., a halo-substituted C₂₋₂₀ alkenyl, such as a halo-substituted C₂₋₆ alkenyl), haloalkynyl (e.g., a halo-substituted C₂₋₂₀ alkynyl, such as a halo-substituted C₂₋₆ alkynyl), perhaloalkyl (e.g., C₁₋₂₀ perhaloalkyl, such as C₁₋₆ perhaloalkyl, such as C₁₋₃ perhaloalkyl), perhaloalkoxy (e.g., C₁₋₂₀ perhaloalkoxy, such as C₁₋₆ perhaloalkoxy), phenyl, aryl (e.g., C₅₋₂₀ aryl, such as C₅₋₁₀ aryl, such as C₅₋₆ aryl), aryloxy (e.g., C₅₋₂₀ aryloxy, such as C₅₋₁₀ aryloxy, such as C₅₋₆ aryloxy), alkoxy (e.g., C₁₋₂₀ alkoxy, such as C₁₋₁₀ alkoxy, such as C₁₋₆ alkoxy), haloalkoxy (e.g., C₁₋₂₀ haloalkoxy, such as C₁₋₁₀ haloalkoxy, such as C₁₋₆ haloalkoxy), oxo, acyloxy (e.g., an acyloxy group including 1-20 carbon atoms, such as 1-10 carbon atoms, such as 1-6 carbon atoms), carbonyl (e.g., C(O) or C═O), carboxyl (e.g., C(O)O), alkylcarbonyl (e.g., C₁₋₂₀ alkylcarbonyl, such as C₁₋₁₀ alkylcarbonyl, such as C₁₋₆ alkylcarbonyl, such as C₁₋₃ alkylcarbonyl), carboxyester (e.g., C(O)OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), carboxamido, cyano (e.g., CN), hydrogen, halogen (e.g., iodine, bromine, chlorine, or fluorine), hydroxy, amino (e.g., NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), alkylamino (e.g., NR′R″ where R′ is alkyl (e.g., C₁₋₂₀ alkyl, such as C₁₋₁₀ alkyl, such as C₁₋₆ alkyl, such as C₁₋₃ alkyl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), arylamino (e.g., NR′R″ where R′ is aryl (e.g., C₅₋₂₀ aryl, such as C₅₋₁₀ aryl, such as C₅₋₆ aryl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), amido (e.g., C(O)NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), nitro (e.g., NO₂), thiol (e.g., SH), alkylthio (e.g., C₁₋₂₀ alkyl substituted with a thiol group, such as C₁₋₁₀ alkyl substituted with a thiol group, such as C₁₋₆ alkyl substituted with a thiol group, such as C₁₋₃ alkyl substituted with a thiol group), haloalkylthio (e.g., C₁₋₂₀ haloalkylthio, such as C₁₋₁₀ haloalkylthio, such as C₁₋₆ haloalkylthio, such as C₁₋₃ haloalkylthio), perhaloalkylthio (e.g., C₁₋₂₀ perhaloalkylthio, such as C₁₋₁₀ perhaloalkylthio, such as C₁₋₆ perhaloalkylthio, such as C₁₋₃ perhaloalkylthio), arylthiol (e.g., C₅₋₂₀ arylthiol, such as C₅₋₁₀ arylthiol, such as C₅₋₆ arylthiol), sulfonate (e.g., S(O)₂OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), sulfonic acid (e.g., S(O)₂OH), trisubstituted silyl (e.g., SiR′R″R* where R′, R″, and R* are independently selected from, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein; in some cases, a trisubstituted silyl can be trimethylsilyl), N₃, SCH₃, C(O)CH₃, CO₂CH₃, CO₂H, pyridinyl, thiophene, furanyl, carbamate, and urea. Additional groups may also be contemplated. Where structurally feasible, two substituents may be joined together to form a fused five-, six-, or seven-membered carbocyclic or heterocyclic ring consisting of zero to three heteroatoms (e.g., N, O, S, etc.), for example forming methylenedioxy or ethylenedioxy. An unsubstituted or substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “unsubstituted or substituted with.”

The terms R, R′, R″, R*, etc., appearing by themselves and without a number designation, unless otherwise defined, refers to a moiety selected from hydrogen, alkyl, cycloalkyl, heteroalkyl, aryl, heteroaryl and heterocycloalkyl, any of which is unsubstituted or substituted (e.g., as described herein). Such R and R′ groups should be understood to be unsubstituted or substituted as defined herein. Whether an R group has a number designation or not, every R group, including R, R′ and Rₙ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

“Bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

Asymmetric centers may exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, atropisomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds provided herein may comprise conformational isomers, which compounds comprise groups that can orient in different conformations in relation to another moiety. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Combination therapy” means the administration of two or more therapeutic agents to treat a therapeutic condition or disorder described in the present disclosure. Such administration encompasses co-administration of these therapeutic agents in a substantially simultaneous manner, such as in a single dose unit (e.g., capsule) having a fixed ratio of active ingredients or in multiple, separate dose units (e.g., capsules) for each active ingredient. In addition, such administration also encompasses use of each type of therapeutic agent in a sequential manner. In either case, the treatment regimen will provide beneficial effects of the drug combination in treating the conditions or disorders described herein.

“KRAS inhibitor” is used herein to refer to a compound that exhibits an IC₅₀ with respect to KRAS activity of no more than about 100 μM and more typically not more than about 50 μM, as measured in the assays described generally herein, such as a surface plasmon resonance KRAS-G12D protein binding assay and/or a KRAS G12D protein-effector protein interaction disruption assay. “IC₅₀” is that concentration of inhibitor which reduces the activity of an enzyme (e.g., KRAS) to half-maximal level. Certain compounds disclosed herein have been discovered to exhibit inhibition against KRAS. In certain embodiments, compounds will exhibit an IC₅₀ with respect to KRAS (e.g., KRAS having a G12D mutation) of no more than about 50 μM; in further embodiments, compounds will exhibit an IC₅₀ with respect to KRAS (e.g., KRAS having a G12D mutation) of no more than about 10 μM; in yet further embodiments, compounds will exhibit an IC₅₀ with respect to KRAS (e.g., KRAS having a G12D mutation) of not more than about 1 μM; in yet further embodiments, compounds will exhibit an IC₅₀ with respect to KRAS (e.g., KRAS having a G12D mutation) of not more than about 200 nM, as measured in the KRAS assay described herein. In some embodiments, compounds will exhibit an IC₅₀ with respect to KRAS (e.g., KRAS having a G12D mutation) of less than about 50 μM, such as less than about 40 μM, 30 μM, 20 μM, 10 μM, 5 μM, 1 μM, 500 nM, 200 nM, 100 nM, 50 nM, or less. In certain embodiments, compounds will exhibit an IC₅₀ with respect to KRAS (e.g., KRAS having a G12D mutation) of less than about 1 μM. In some embodiments, a KRAS inhibitor has inhibitory activity against KRAS having a G12D mutation that exceeds its inhibitory activity against KRAS having another mutation, such as a G12C, G12R, G12S, G12A, or G12V mutation. For example, in some embodiments, a KRAS inhibitor provided herein has at least two-fold, five-fold, ten-fold, twenty-fold, or higher inhibitory activity against KRAS having a G12D mutation relative to KRAS having another mutation such as a G12C, G12R, G12S, G12A, or G12V mutation, such as a G12C mutation. In some embodiments, a KRAS inhibitor provided herein has greater inhibitory activity against active KRAS having a G12D mutation than against an inactive KRAS having a G12D mutation. In some embodiments, a KRAS inhibitor provided herein has lower inhibitory activity against active KRAS having a G12D mutation than against an inactive KRAS having a G12D mutation. In some embodiments, a KRAS inhibitor provided herein has similar inhibitory activity against active and inactive KRAS having a G12D mutation. In some embodiments, a KRAS inhibitor provided herein has inhibitory activity against a K-RAS4a splice variant. In some embodiments, a KRAS inhibitor provided herein has inhibitory activity against a K-RAS4b splice variant. In some embodiments, a KRAS inhibitor provided herein has inhibitory activity against both K-RAS4a and K-RAS4b splice variants.

“Therapeutically effective amount” refers to an amount of a compound or of a pharmaceutical composition useful for treating or ameliorating an identified disease, disorder, or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

“Treat,” “treating,” and “treatment” refer to any indicia of success in the treatment or amelioration of an injury, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being. The treatment or amelioration of symptoms can be based on objective or subjective parameters, including the results of a physical examination, neuropsychiatric exams, and/or a psychiatric evaluation. Treatment may also be preemptive in nature; i.e., it may include prevention of a disease, disorder, or condition, prevention of onset of one or more symptoms of a disease, disorder, or condition, and/or prevention of escalation of a disease, disorder, or condition. Prevention of a disease, disorder, or condition may involve complete protection from disease, and/or prevention of disease progression (e.g., to a later stage of the disease, disorder, or condition). For example, prevention of a disease may not mean complete foreclosure of any effect related to the diseases at any level, but instead may mean prevention of the symptoms of a disease, disorder, or condition to a clinically significant or detectable level.

“Patient” or “subject” refers to a living organism suffering from or prone to a disease, disorder, or condition that can be treated by administration of a compound or pharmaceutical composition as provided herein. Non-limiting examples include humans, rats, mice, rabbits, hamsters, guinea pigs, hamsters, cats, dogs, non-human primates (e.g., monkeys), goats, pigs, sheep, cows, deer, horses, and other non-mammalian animals. Examples of mammals that can be treated by administration of a compound or pharmaceutical composition provided herein include, for example, rodents (e.g., rats, mice, squirrels, guinea pigs, hamsters, etc.), lagomorphs (e.g., rabbits, hares, etc.), primates (e.g., monkeys, apes, etc.), bovines (e.g., cattle), odd-toed ungulates (e.g., horses), even-toed ungulates (e.g., bovines such as cattle, ovine such as sheep, caprine such as goats, porcine such as pigs, etc.), and marsupials (e.g., kangaroo, wallaby, wallaroo, sugar glider, etc.). In some embodiments, the patient or subject is human. In some embodiments, the patient or subject is a companion animal such as a cat or dog. In some embodiments, the patient or subject is a farm animal such as a goat, sheep, cow, pig, or horse. In some embodiments, the patient or subject is an exotic animal such as a primate (e.g., monkey), marsupial (e.g., kangaroo, wallaby, wallaroo, sugar glider, etc.), or a non-domesticated or hybrid cat or dog.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent, or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors, and colors. One of skill in the art will recognize that other pharmaceutical excipients are useful in the present disclosure.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug.

The compounds disclosed herein can exist as therapeutically acceptable salts. The present disclosure includes compounds provided herein in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

“KRAS G12D-positive cancer” refers to a cancer characterized by a KRAS G12D mutation.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a KRAS G12D inhibitor, as defined herein, and an additional agent, which additional agent may be an agent configured to treat a disease, disorder, or condition or a symptom thereof. The effect can be, for example, slowing the symptomatic progression of a proliferative disease, such as cancer, particularly lung cancer, or symptoms thereof. Analogously, a “synergistically effective amount” refers to the amount needed to obtain a synergistic effect

“A,” “an,” or “a(n)”, when used in reference to a group of substituents or “substituent group” herein, mean at least one. For example, where a compound is substituted with “an” alkyl or aryl, the compound is unsubstituted or substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### Compounds

In an aspect, the present disclosure provides a compound represented by Formula I:

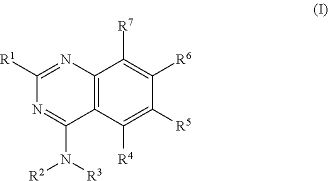

or a salt (e.g., pharmaceutically acceptable salt), ester, tautomer, prodrug zwitterionic form, or stereoisomer thereof, wherein:
R¹ is selected from —OR⁸, a 4-6 membered heterocycle including a nitrogen atom, and H, wherein the heterocycle is unsubstituted or substituted with one or more R¹⁶;
R² is selected from H, a 3-6 membered carbocycle, and C₁₋₆ alkyl, wherein the 3-6 membered carbocycle or the C₁₋₆ alkyl is unsubstituted or substituted with one or more R¹³;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from H, halogen, —OR¹², —CN, C₁₋₆alkyl, C₂₋₆alkenyl, C₂₋₆alkynyl, a 3-6 membered carbocycle, a 3-6 membered heterocycle, a 5-6 membered heteroaryl, and phenyl, wherein any C₁₋₆alkyl is unsubstituted or substituted with one or more R¹³; and wherein any carbocycle, heterocycle, heteroaryl, and phenyl is unsubstituted or substituted with one or more R¹⁴;
R⁶ is a phenyl, monocyclic heteroaryl, bicyclic aryl, or bicyclic heteroaryl, wherein any phenyl, monocyclic heteroaryl, bicyclic aryl, and bicyclic heteroaryl is unsubstituted or substituted with one or more R⁵;
R⁷ is selected from halogen, —OR¹², —CN, and H;
R⁸ is selected from H, heterocycle, and alkylheterocycle, wherein the heterocycle or alkylheterocycle is unsubstituted or substituted with one or more R¹⁶, and wherein an alkyl moiety of any alkylheterocycle is selected from C₁₋₆ alkyl;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from halogen, —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²¹;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, —OR¹², —CN, —N(R¹²)₂, and C₁₋₆alkyl,
wherein any C₁₋₆alkyl is unsubstituted or substituted with one or more R¹³;
each R¹⁵ is independently selected from halogen, —OR¹², —CN, —N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, —OR¹², and 3-6 membered heterocycle, wherein any C₁₋₆alkyl is unsubstituted or substituted with one or more R¹³;
each R¹⁷ is independently selected from C₁₋₆ alkyl and H;
each R¹⁷ is independently selected from —OH, —OC₁₋₆alkyl, —CN, —NH₂, —NHC₁₋₆alkyl, and halogen;
each R¹⁹ is independently selected from C₁₋₆ alkyl, 3-6 membered heterocycle, and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, halogen, and 3-6 membered carbocycle;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, the present disclosure provides a compound of Formula I, or a salt (e.g., a pharmaceutically acceptable salt) thereof.

In some embodiments, R¹ is —OR⁸. In some embodiments, R⁸ is a heterocycle. In some embodiments, R⁸ is an alkylheterocycle. In some embodiments, R⁸ is an alkylheterocycle, wherein the alkyl moiety of the alkylheterocycle is selected from C₁₋₆ alkyl. In some embodiments, R⁸ is —CH₂(heterocycle). In some embodiments, a heterocycle or a heterocycle of an alkylheterocycle comprises 4-8 members including at least one heteroatom (e.g., 1-2 heteroatoms) selected from N, O, and S. In some embodiments, R⁸ comprises a heterocycle including at least one nitrogen atom. In some embodiments, R⁸ comprises a 4-8 membered heterocycle including at least one nitrogen atom. In some embodiments, a heterocycle or a heterocycle of an alkylheterocycle is a 4-6 membered monocyclic heterocycle having 1-2 heteroatoms independently selected from nitrogen, oxygen, and sulfur. In some embodiments, a heterocycle or a heterocycle of an alkylheterocycle is an 8-membered bicyclic heterocycle having 1-2 heteroatoms independently selected from nitrogen, oxygen, and sulfur. In some embodiments, the heterocycle is substituted with one or more R¹⁶. In some embodiments, at least one R¹⁶ is —OR¹², wherein R¹² is independently selected from C₁₋₆ alkyl, C₂₋₆ alkenyl, and H. In some embodiments, at least one R¹⁶ is —OCH₃. In some embodiments, at least one R¹⁶ is halogen (e.g., F). In some embodiments, at least one R¹⁶ is C₁₋₆alkyl unsubstituted or substituted with one or more R¹³. In some embodiments, each R¹⁶ is independently selected from halogen, C₁₋₆alkyl, and —OR¹², wherein any C₁₋₆alkyl is unsubstituted or substituted with one or more R¹³. In some embodiments, each R¹⁶ is independently selected from —F, —CH₃, —CH₂CH₂F, —CH₂CHF₂, —CH₂CH₂CN, —OCH₃, and —OCHF₂.

In some embodiments, R¹ is selected from:

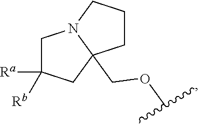

wherein R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, R¹ is selected from:

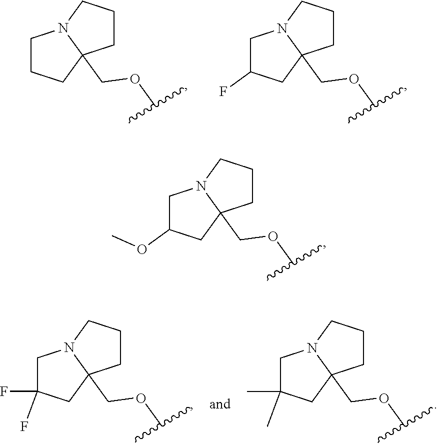 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, R¹ is selected from:

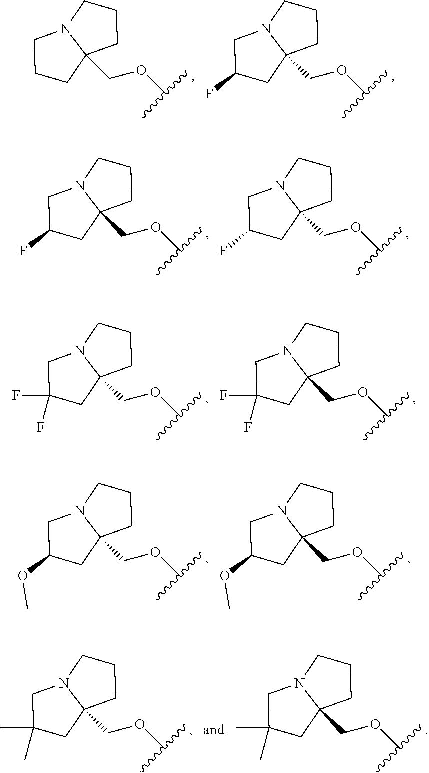

In some embodiments, R¹ is selected from:

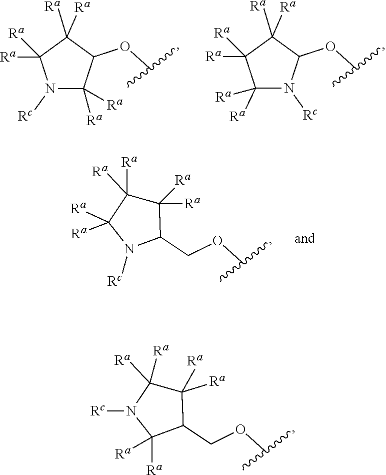

wherein each R^{a} is independently selected from halogen, C₁₋₆ alkyl, —OR¹², and H; and wherein R^{c} is selected from C₁₋₆ alkyl, wherein a C₁₋₆ alkyl of a R^{a} or R^{c} is unsubstituted or is substituted with one or more R¹³. In some embodiments, one R^{a} is selected from halogen, C₁₋₆ alkyl, and —OR¹², and the other R^{a}s are H. In some embodiments, one R^{a} is halogen (e.g., F). In some embodiments, two R^{a}'s are halogen (e.g., F). In some embodiments, one R^{a} is —OR¹² (e.g., —OCH₃ or —CHF₂). In some embodiments, one R^{a} is C₁₋₆ alkyl (e.g., methyl). In some embodiments, two R^{a}'s are C₁₋₆ alkyl (e.g., methyl). In some embodiments, R^{c} is selected from —CH₃, —CH₂CH₂F, —CH₂CHF₂, and —CH₂CH₂CN. In some embodiments, R¹ is selected from:

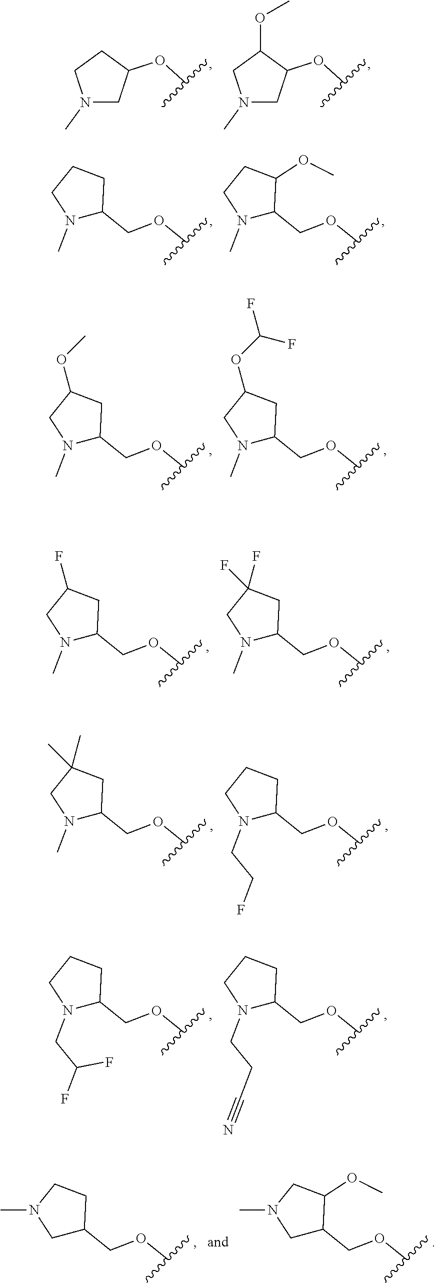

In some embodiments, R¹ is selected from:

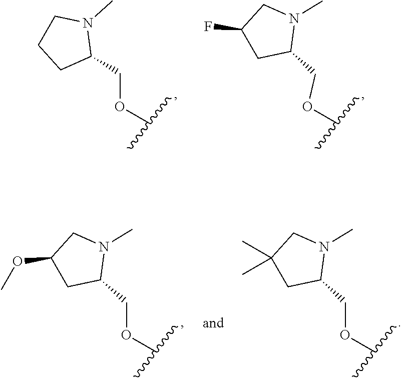

In some embodiments, R¹ is selected from:

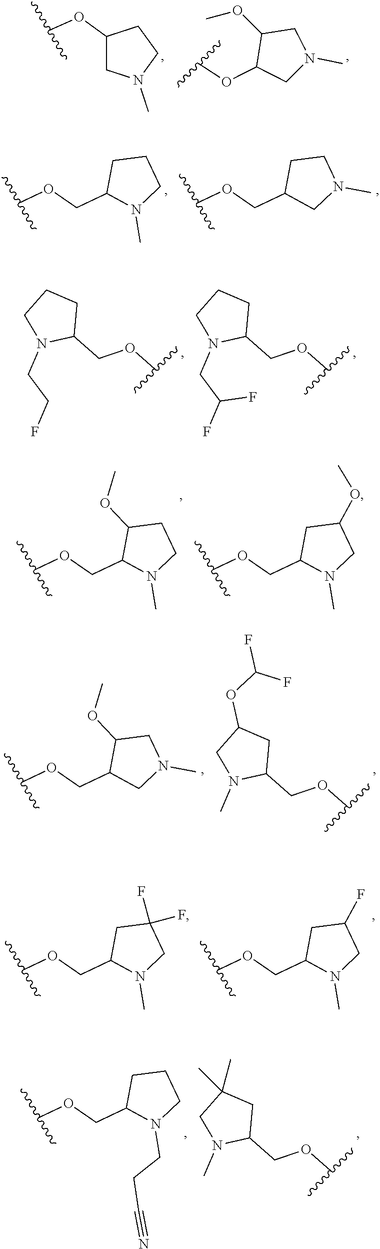

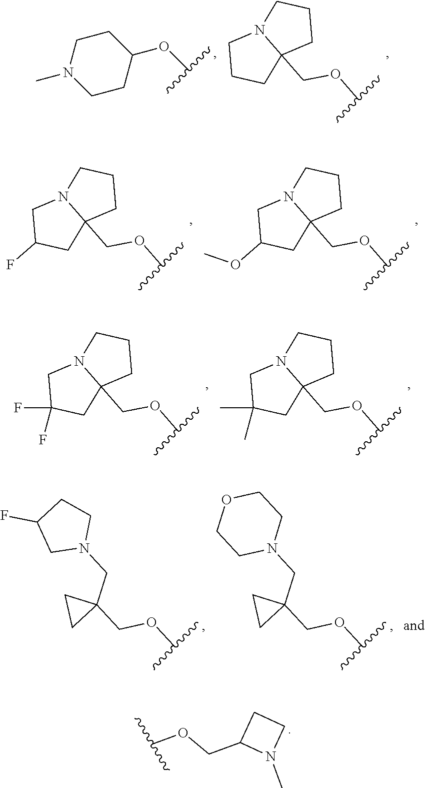

In some embodiments, R¹ is selected from:

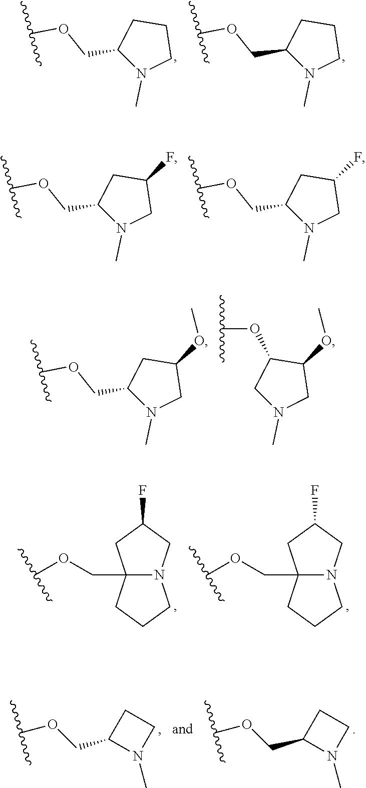

In some embodiments, R¹ is a 4-6 membered heterocycle including a nitrogen atom, which heterocycle is unsubstituted or substituted with one or more R¹⁶. In some such embodiments, R¹⁶ is selected from —N(R¹²)₂, C₁₋₆alkyl, and 3-6 membered heterocycle. In some embodiments, R¹⁶ is —N(C₁₋₆alkyl)₂, e.g., —N(CH₃)₂. In some embodiments, R¹⁶ is C₁₋₆alkyl (e.g., methyl). In some embodiments, R¹⁶ is a bicyclic 6-membered heterocycle having 1 nitrogen atom. In some embodiments, R¹ is selected from:

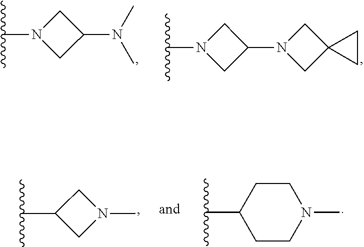

In some embodiments, R¹ is H.

In some embodiments, R² is H. In some embodiments, R² is C₁₋₆ alkyl unsubstituted or substituted with one or more R¹³. In some such embodiments, each R¹³ is independently selected from —OR²² (e.g., —OH) and —CN. In some embodiments, R² is C₁₋₆ alkyl. In some embodiments R² is selected from —CH₃, —CH₂CH₃, —CH₂CH₂OH, —CH₂CH₂CN, and —CH(CH₃)₂. In some embodiments, R² is a 3-6 membered carbocycle. In some embodiments, R² is cyclopropyl.

In some embodiments, R³ is C₁₋₆ alkyl, which C₁₋₆alkyl is substituted with one or more R⁹. In some embodiments, R³ is C₁₋₆ alkyl substituted with —N(R¹⁷)₂, wherein each R¹⁷ is independently selected from C₁₋₆ alkyl and H. In some embodiments, R³ is C₁₋₆ alkyl substituted with —NH₂, —NH(CH₃), or —N(CH₃)₂. In some embodiments, R³ is C₁₋₆ alkyl substituted with —N(R¹⁷)C(O)C₁₋₆alkyl (e.g., —N(H)C(O)CH₃). In some embodiments, R³ is C₁₋₆ alkyl substituted with —OR¹⁷ (e.g., —OH).

In some embodiments, R³ is selected from:

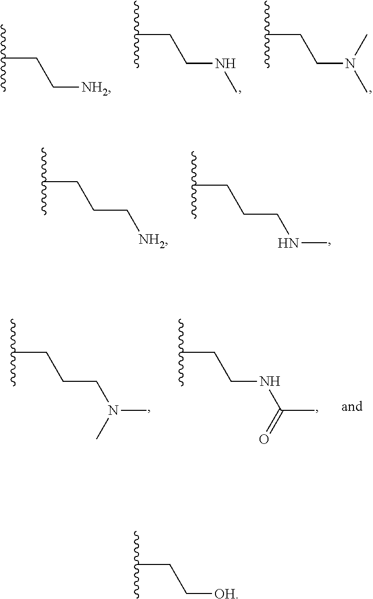

In some embodiments, R³ is a carbocycle, which carbocycle is unsubstituted or substituted with one or more R¹⁰. In some embodiments, R³ is a 4-6 membered carbocycle, which carbocycle is unsubstituted or substituted with one or more R¹⁰. In some embodiments, R³ is a multicyclic carbocycle (e.g., a 5-6 membered bicyclic carbocycle). In some embodiments, R³ is a monocyclic carbocycle (e.g., a 4-6 membered monocyclic carbocycle). In some embodiments, R³ is a carbocycle that is substituted with —N(R¹⁹)₂, wherein each R¹⁹ is independently selected from unsubstituted or substituted C₁₋₆ alkyl and H. In some embodiments, R³ is a carbocycle that is substituted with —NH₂.

In some embodiments, R³ is selected from:

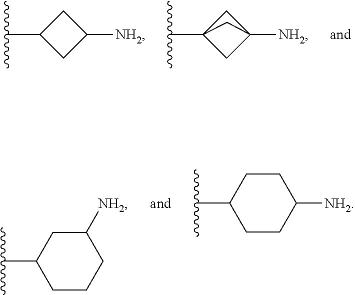 [SMILES: *C1CC(N)C1 ; *C12CC(N)(C1)C2 ; *C1CCCC(N)C1 ; *C1CCC(N)CC1]

In some embodiments, R³ is a heterocycle, which heterocycle is unsubstituted or substituted with one or more R¹⁰. In some embodiments, R³ is a 4-8 membered heterocycle having 1-2 heteroatoms independently selected from nitrogen, oxygen, and sulfur, which heterocycle is unsubstituted or substituted with one or more R¹⁰. In some embodiments, R³ is a multicyclic heterocycle (e.g., a 6-8 membered bicyclic heterocycle). In some embodiments, R³ is a monocyclic heterocycle (e.g., a 4-6 membered monocyclic heterocycle). In some embodiments, the heterocycle includes one or more nitrogen atoms. In some embodiments, R³ is a heterocycle that is unsubstituted or substituted with one or more R¹⁰, wherein each R¹⁰ is independently selected from halogen, —N(R¹⁹)₂, —C(O)R¹⁹, —C(O)N(R¹⁹)₂, —C(O)(C₁₋₆alkyl)N(R¹⁹)₂, and C₁₋₆alkyl, wherein any C₁₋₆alkyl is unsubstituted or substituted with one or more R²⁰. In some embodiments, R³ is a heterocycle that is unsubstituted or substituted with one or more groups selected from —F, —CH₃, —CH₂F, —CH₂CN, —C(O)CH₃, —C(O)CH₂NH₂, —C(O)NH₂, and

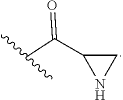 [SMILES: *C(=O)C1CN1]

In some embodiments, R³ is a heterocycle that is substituted with —N(R¹⁹)₂, wherein each R⁹ is independently selected from unsubstituted or substituted C₁₋₆ alkyl and H. In some embodiments, R³ is a heterocycle that is substituted with —NH₂.

In some embodiments, R³ is selected from:

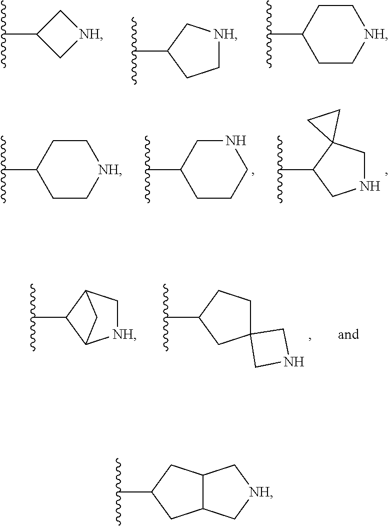

any of which is unsubstituted or substituted with one or more R¹⁰.

In some embodiments, R³ is selected from:

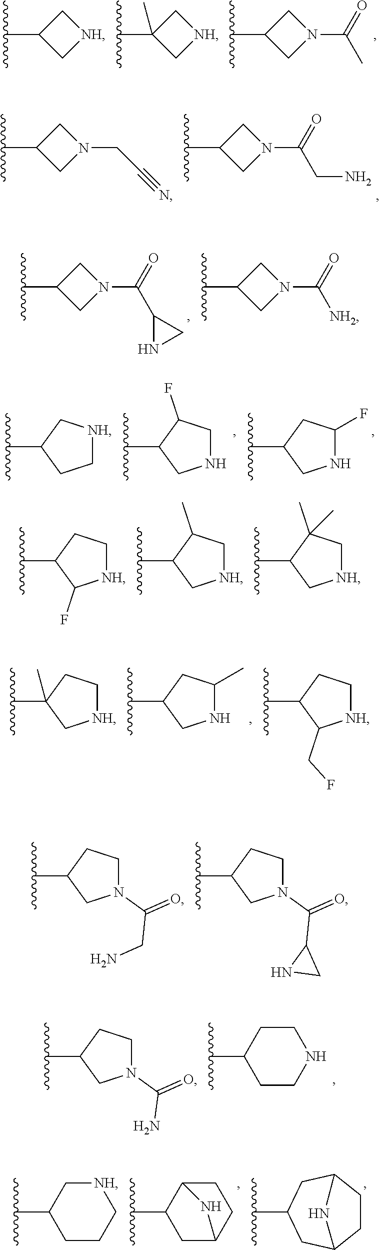

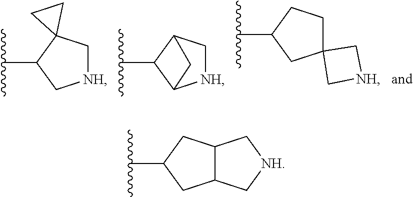

In some embodiments, R³ is selected from:

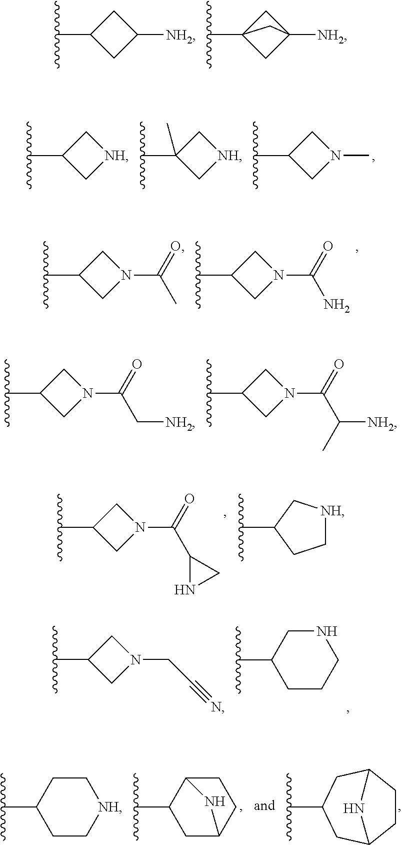

any of which is unsubstituted or substituted with one or more R¹⁰.

In some embodiments, R² or R³ includes an amino moiety (e.g., —NRR′). In some embodiments, R² or R³ is substituted with an amino moiety (i.e., —N(R¹⁷)₂ or —N(R¹⁹)₂).

In some embodiments, R² and R³, together with the atom (e.g., nitrogen atom) to which they are attached, form a heterocycle that is unsubstituted or substituted with one or more R¹¹. In some embodiments, R² and R³, together with the atom (e.g., nitrogen atom) to which they are attached, form a 4-9 membered heterocycle having 0-2 additional heteroatoms independently selected from nitrogen, oxygen, and sulfur, wherein the heterocycle is unsubstituted or substituted with one or more R¹¹. In some embodiments, R² and R³, together with the atom (e.g., nitrogen atom) to which they are attached, form a 4-7 membered monocyclic heterocycle having 0-1 additional heteroatoms independently selected from nitrogen, oxygen, and sulfur, wherein the heterocycle is unsubstituted or substituted with one or more R¹¹. In some embodiments, R² and R³, together with the atom (e.g., nitrogen atom) to which they are attached, form a 7-9 membered bicyclic heterocycle having 0-2 additional heteroatoms independently selected from nitrogen, oxygen, and sulfur, wherein the heterocycle is unsubstituted or substituted with one or more R¹¹. In some such embodiments, each R¹¹ is independently selected from —N(R¹⁹)₂, —C(O)R¹⁹, —C(O)N(R¹⁹)₂, —C(O)(C₁₋₆alkyl)N(R¹⁹)₂, and C₁₋₆alkyl, wherein any C₁₋₆alkyl is unsubstituted or substituted with one or more R²⁰. In some embodiments, each R¹¹ is independently selected from —NH₂, —NHCH₃, —C(O)CH₃, —C(O)NH₂, —C(O)CH₂NH₂, —CH₃, —CH₂CH₃, —CH(CH₃)₂, —CH₂CN, —CH₂C(O)NH₂, —C(NH)NHCN, —CH₂OH, and

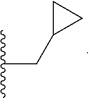 [SMILES: *CC1CC1]

In some embodiments, R² and R³, together with the atom to which they are attached, form a heterocycle selected from:

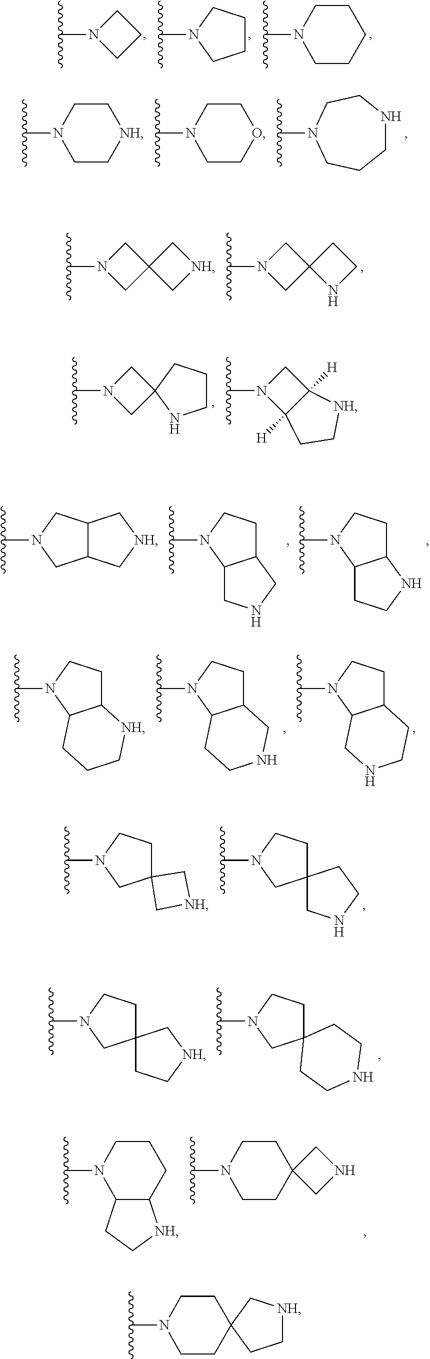

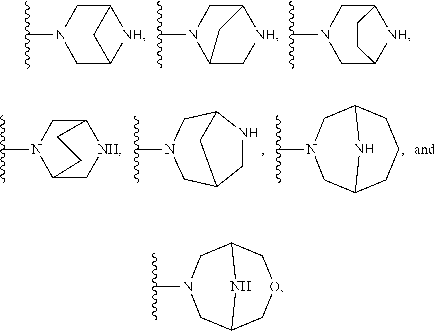

any of which is unsubstituted or substituted with one or more R¹¹.

In some embodiments, R² and R³, together with the atom to which they are attached, form a heterocycle selected from:

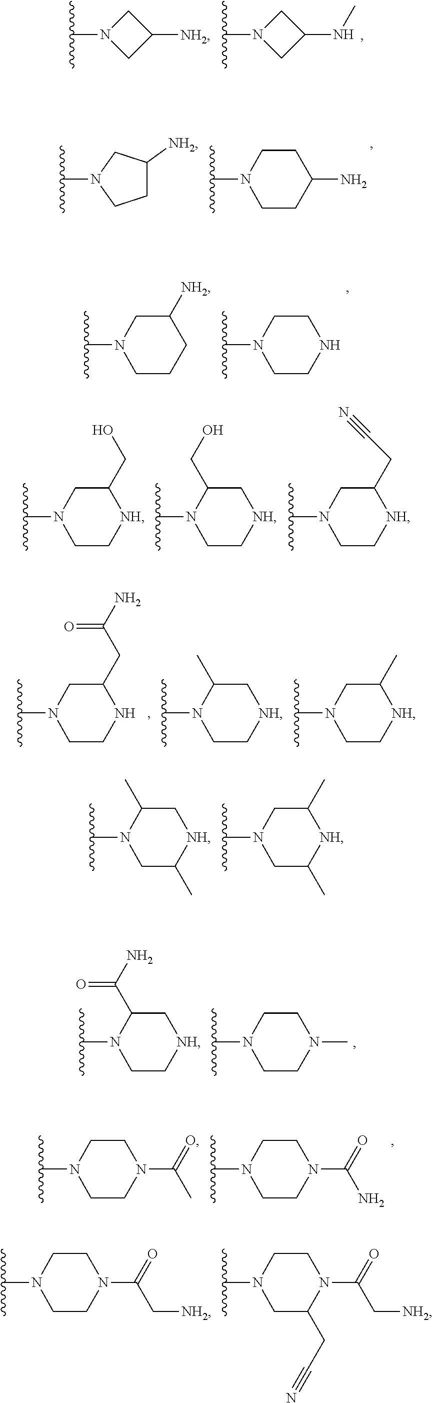

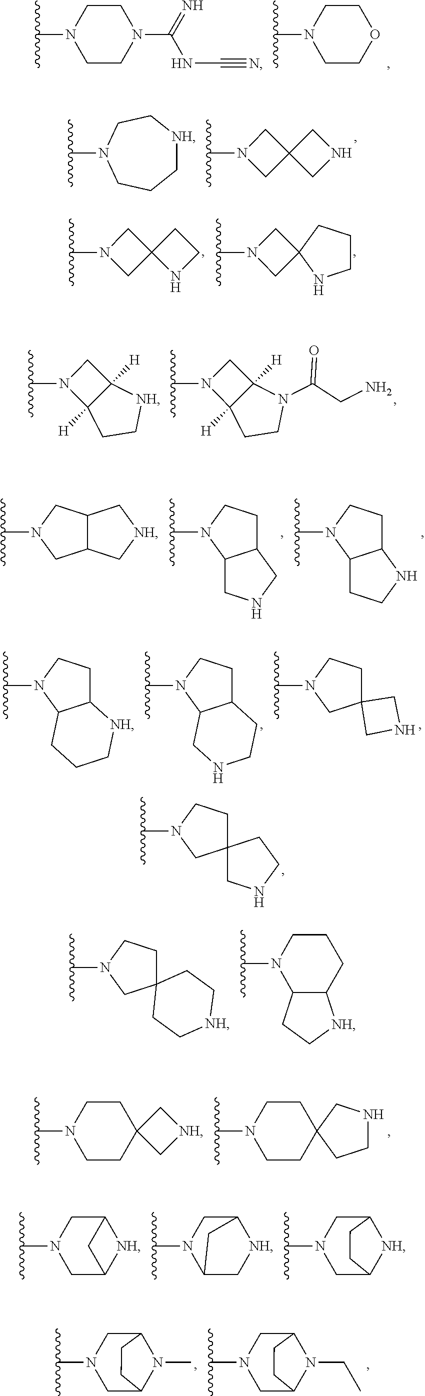

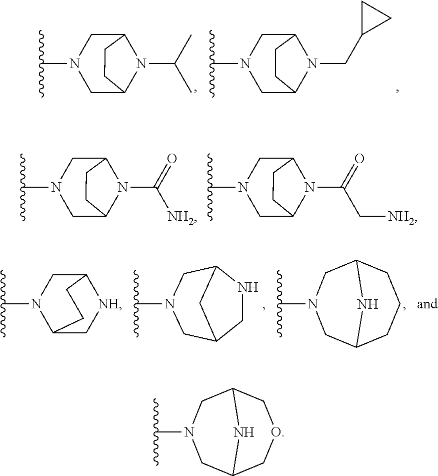

In some embodiments, R² and R³, together with the atom to which they are attached, form a heterocycle having the structure:

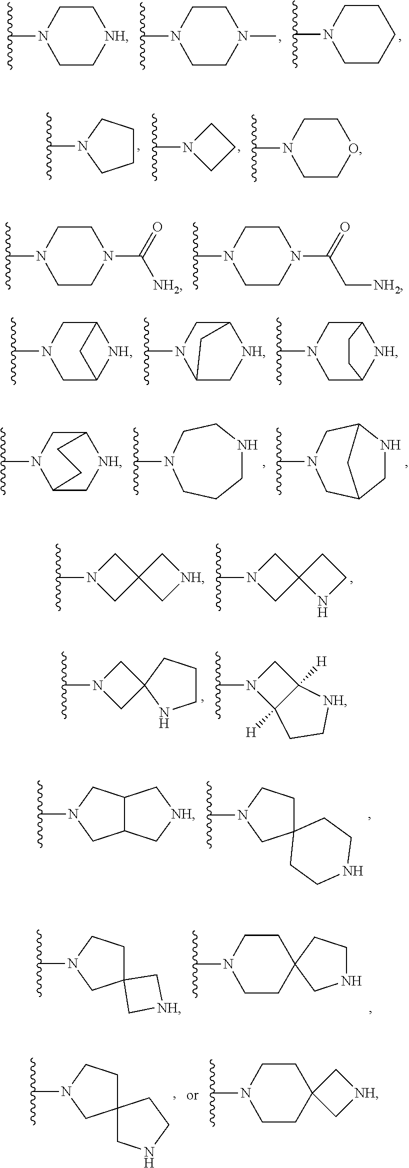

any of which is unsubstituted or substituted with one or more R¹¹. In some embodiments, R² and R³, together with the atom to which they are attached, form a heterocycle having the structure:

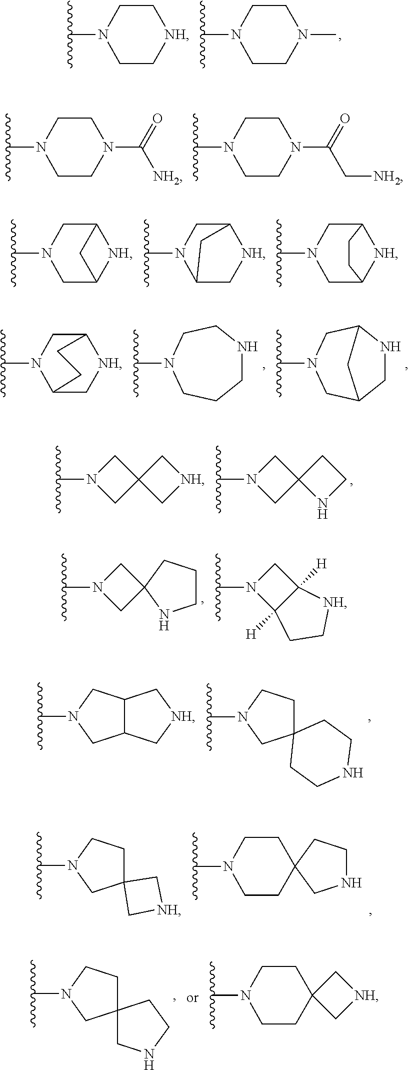

any of which is unsubstituted or substituted with one or more R¹¹. In some embodiments, R² and R³, together with the atom to which they are attached, form a heterocycle having the structure:

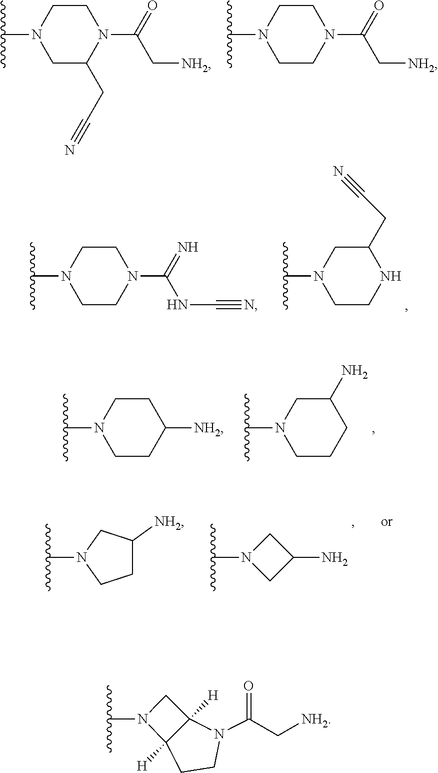

In some embodiments, R² and R³, together with the atom to which they are attached, form a heterocycle having the structure:

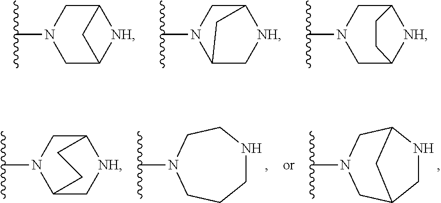

any of which is unsubstituted or substituted with one or more R¹¹.

In some embodiments, R² and R³, together with the atom to which they are attached, form a heterocycle having the structure:

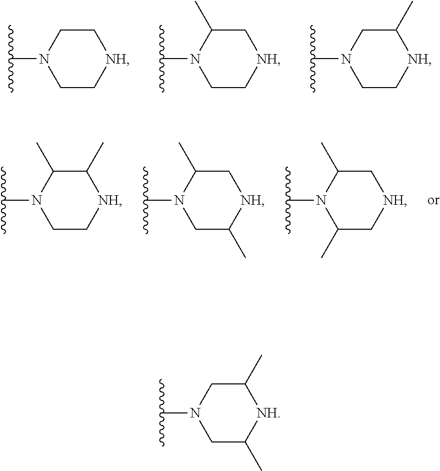

In some embodiments, R² and R³, together with the nitrogen atom to which they are attached, form a heterocycle that is unsubstituted or substituted with one or more R¹¹, which heterocycle includes an additional nitrogen atom and/or is substituted with a group including an amino moiety (e.g., —N(R¹⁹)₂).

In some embodiments, R⁴ is H. In some embodiments, R⁴ is a halogen. In some embodiments, R⁴ is —OR¹². In some embodiments, R⁴ is —OCH₃.

In some embodiments, R⁵ is a 3-6 membered carbocycle unsubstituted or substituted with one or more R¹⁴. In some embodiments, R⁵ is a 3-4 membered carbocycle unsubstituted or substituted with one or more R¹⁴ (e.g., one or more —OR¹² or —CN). In some embodiments, R⁵ is a 3-6 membered heterocycle (e.g., a 3-6 membered heterocycle having 1-2 heteroatoms independently selected from nitrogen, oxygen, and sulfur) unsubstituted or substituted with one or more R¹⁴. In some embodiments, R⁵ is a 5 or 6 membered aryl or heteroaryl moiety unsubstituted or substituted with one or more R¹⁴. In some embodiments, R⁵ is phenyl. In some embodiments, R⁵ is a 5-6 membered heteroaryl unsubstituted or substituted with one or more R¹⁴ (e.g., C₁₋₆alkyl). In some embodiments, R⁵ is a pyridyl, furanyl, or imidazolyl, each unsubstituted or substituted with one or more R¹⁴ (e.g., C₁₋₆alkyl). In some embodiments, R⁵ is a furanyl.

In some embodiments, R⁵ is selected from C₁₋₆alkyl that is unsubstituted or substituted with one or more R¹³. In some embodiments, R⁵ is selected from C₁₋₆alkyl that is unsubstituted, such as methyl or ethyl. In some embodiments, R⁵ is selected from C₁₋₆alkyl that is substituted with one or more halogens or —CN. In some embodiments, R⁵ is selected from —CF₃, —CF₂H, and —CH₂CN. In some embodiments, R⁵ is selected from —CH₃, —CH₂CH₃, —CF₂H, —CF₃, —CF₂CH₃, and —CH₂CN.

In some embodiments, R⁵ is C₂₋₆alkenyl. In some embodiments, R⁵ is C₂₋₆alkynyl (e.g., ethynyl).

In some embodiments, R⁵ is selected from —OR¹², wherein R¹² is selected from C₁₋₆alkyl that is unsubstituted or substituted with one or more R¹³. In some embodiments, R⁵ is —OCH₃, —OCF₃, or —OCF₂H.

In some embodiments, R⁵ is —CN.

In some embodiments, R⁵ is a halogen. In some embodiments, R⁵ is Cl or F.

In some embodiments, R⁵ is hydrogen.

In some embodiments, R⁷ is a halogen. In some embodiments, R⁷ is Cl or F. In some embodiments, R⁷ is —OR¹², wherein R¹² is selected from H and C₁₋₆alkyl that is unsubstituted or substituted with one or more R¹³. In some embodiments, R⁷ is —OH, —OCH₃, or —OCH₂CF₃. In some embodiments, R⁷ is —CN. In some embodiments, R⁷ is hydrogen.

In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, R⁶ is a bicyclic aryl or bicyclic heteroaryl, wherein the aryl or heteroaryl are unsubstituted or substituted with one or more R¹⁵. In some embodiments, R⁶ is a bicyclic aryl or bicyclic heteroaryl, wherein the aryl or heteroaryl are substituted with one or more R¹⁵.

In some embodiments, R⁶ is a bicyclic aryl substituted with one or more R¹⁵. In some embodiments, R⁶ is naphthyl substituted with one or more R¹⁵. In some embodiments, R⁶ is selected from:

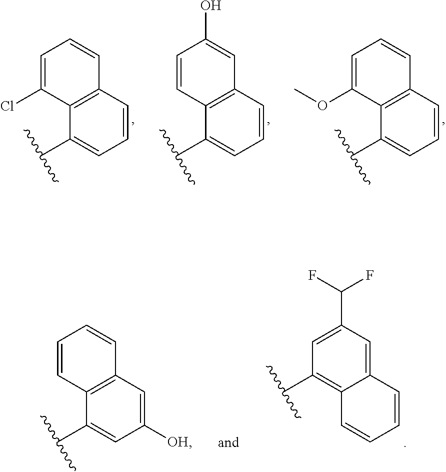

In some embodiments, R⁶ is a 9-10 membered heteroaryl having 1-3 heteroatoms independently selected from nitrogen, oxygen, and sulfur that is unsubstituted or substituted with one or more R¹⁵. In some embodiments, R⁶ is a bicyclic heteroaryl substituted with one or more R¹⁵. In some embodiments, R⁶ is a 9-membered heteroaryl having 1-3 heteroatoms independently selected from nitrogen, oxygen, and sulfur and substituted with one or more R¹⁵. In some such embodiments, at least one R¹⁵ is —N(R¹²)₂ (e.g., —NH₂). In some embodiments, at least one R¹⁵ is a halogen (e.g., F). In some embodiments, each R¹⁵ is independently selected from halogen, —CN, and —N(R¹²)₂. In some embodiments, R⁶ is substituted with at least two R¹⁵ (e.g., at least a halogen and —NH₂).

In some embodiments, R⁶ is selected from:

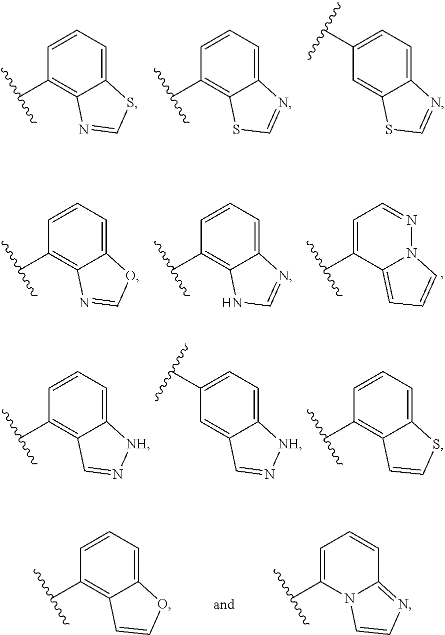

any of which is unsubstituted or substituted with one or more R¹⁵.

In some embodiments, R⁶ is selected from:

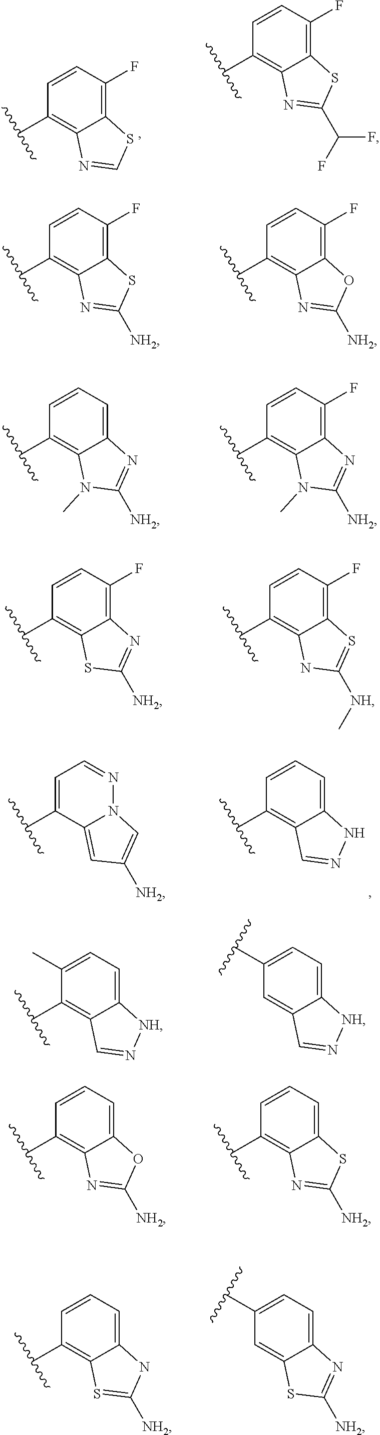

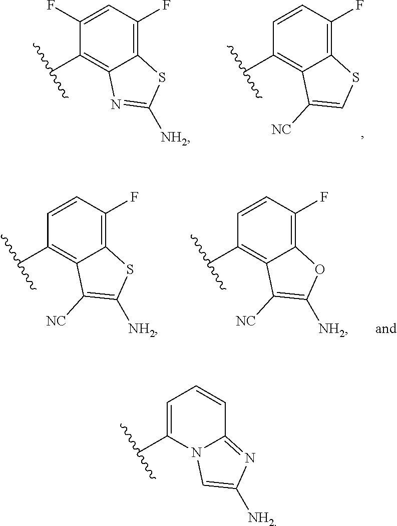

In some embodiments, R⁶ is selected from:

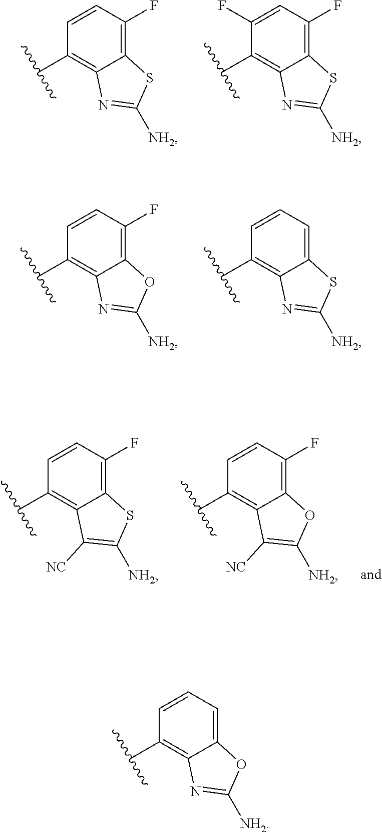

In some embodiments, R⁶ is:

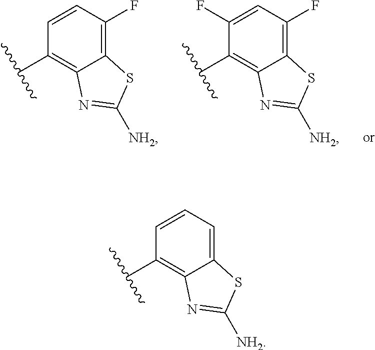 [SMILES: *c1ccc(F)c2sc(N)nc12 ; *c1c(F)cc(F)c2sc(N)nc12 ; *c1cccc2sc(N)nc12]

In some embodiments, R⁶ is

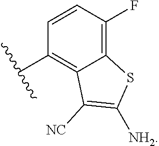 [SMILES: *c1ccc(F)c2sc(N)c(C#N)c12]

In some embodiments, R⁶ is phenyl or monocyclic heteroaryl, wherein the phenyl or heteroaryl is unsubstituted or substituted with one or more R¹⁵.

In some embodiments, R⁶ is phenyl unsubstituted or substituted with one or more R¹⁵. In some such embodiments, each R¹⁵ is independently selected from halogen, —OR¹², —CN, and —N(R¹²)₂. In some embodiments, R⁶ is selected from:

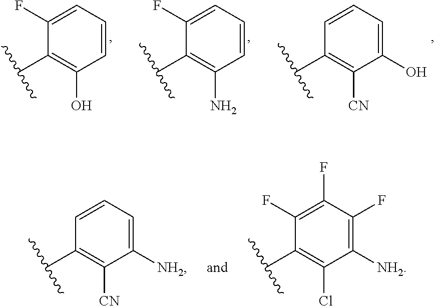 [SMILES: *c1c(O)cccc1F ; *c1c(N)cccc1F ; *c1cccc(O)c1C#N ; *c1cccc(N)c1C#N ; *c1c(F)c(F)c(F)c(N)c1Cl]

In some embodiments, R⁶ is a monocyclic 5-6 membered heteroaryl unsubstituted or substituted with one or more R¹⁵. In some embodiments, R⁶ is pyridyl unsubstituted or substituted with one or more R¹⁵. In some such embodiments, each R¹⁵ is independently selected from —N(R¹²)₂ and C₁₋₆alkyl, wherein any C₁₋₆alkyl is unsubstituted or substituted with one or more R¹³. In some embodiments, R⁶ is

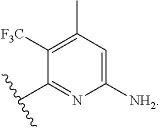 [SMILES: *c1nc(N)cc(C)c1C(F)(F)F]

In some embodiments, (i) R² is selected from C₁₋₆alkyl and a 3-6 membered carbocycle, wherein the C₁₋₆alkyl and a 3-6 membered carbocycle is unsubstituted or is substituted with one or more R¹³; and (ii) R³ is selected from a carbocycle and a heterocycle, wherein the carbocycle or heterocycle is unsubstituted or substituted with one or more R¹⁰. In some embodiments, R⁴ is H. In some embodiments, R⁷ is a halogen (e.g., F). In some embodiments, R⁵ is selected from C₁₋₆alkyl, a halogen, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴. In some embodiments, R⁶ is a bicyclic heteroaryl substituted with one or more R¹⁵. In some embodiments, R⁶ is.

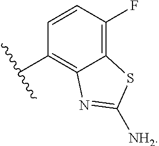 [SMILES: *c1ccc(F)c2sc(N)nc12]

In some embodiments, R¹ is —OR⁸, wherein R⁸ is a heterocycle or an alkylheterocycle. In some embodiments, R¹ is selected from:

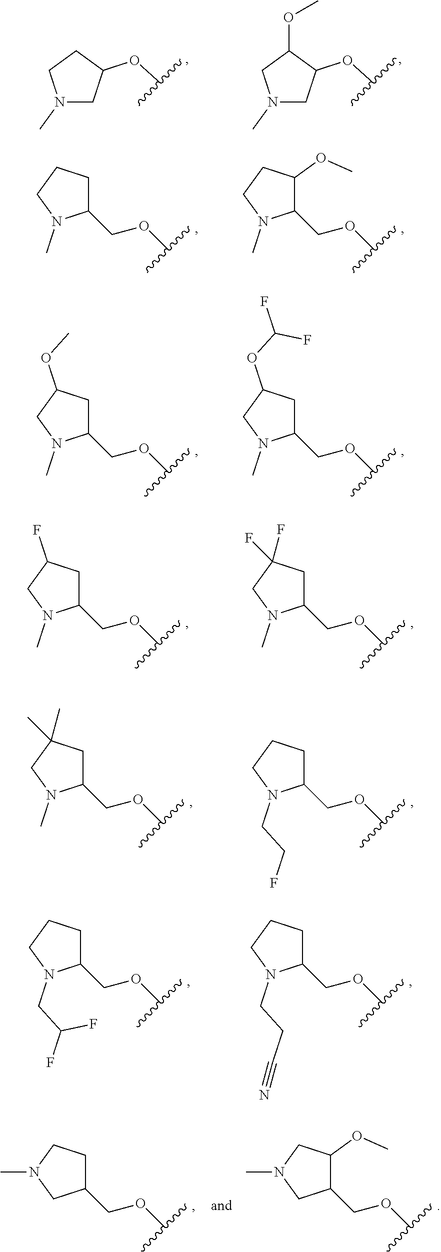

In some embodiments, R¹ is selected from:

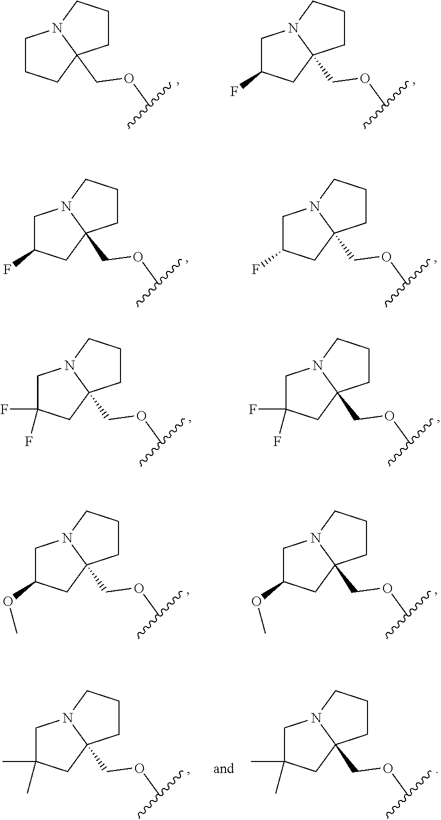

In some embodiments, (i) R² is selected from C₁₋₆alkyl (e.g., methyl or ethyl) that is unsubstituted or is substituted with one or more R¹³; and (ii) R³ is selected from a carbocycle that is unsubstituted or substituted with one or more R¹⁰. In some embodiments, (i) R² is selected from C₁₋₆alkyl (e.g., methyl or ethyl) that is unsubstituted or is substituted with one or more R¹³; and (ii) R³ is selected from a heterocycle that is unsubstituted or substituted with one or more R¹⁰. In some embodiments, (i) R² is a 3-6 membered carbocycle (e.g., cyclopropyl) that is unsubstituted or is substituted with one or more R¹³; and (ii) R³ is selected from a carbocycle that is unsubstituted or substituted with one or more R¹⁰. In some embodiments, (i) R² is a 3-6 membered carbocycle (e.g., cyclopropyl) that is unsubstituted or is substituted with one or more R¹³; and (ii) R³ is selected from a heterocycle that is unsubstituted or substituted with one or more R¹⁰.

In an aspect, the present disclosure provides a compound represented by Formula IA:

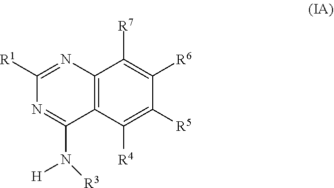

or a salt (e.g., pharmaceutically acceptable salt), ester, tautomer, prodrug, zwitterionic form, or stereoisomer thereof, wherein R¹, R³, R⁴, R⁵, R⁶, and R⁷ are as defined above for Formula I and described in classes and subclasses herein, both singly and in combination. In some embodiments, the present disclosure provides a compound of Formula IA, or a salt (e.g., a pharmaceutically acceptable salt) thereof.

In some embodiments, the present disclosure provides a compound of Formula IA, wherein:
R¹ is selected from —OR⁸, a 4-6 membered heterocycle including a nitrogen atom, and H, wherein the heterocycle is unsubstituted or substituted with one or more R¹⁶;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶, and wherein an alkyl moiety of any alkylheterocycle is selected from C₁₋₆ alkyl;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen; each R¹⁴ is independently selected from halogen, N(R¹²)₂, and C₁₋₆alkyl, wherein any C₁₋₆alkyl is unsubstituted or substituted with one or more R¹³; each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, R³ is selected from a carbocycle, and a heterocycle, wherein the carbocycle or heterocycle is unsubstituted or substituted with one or more R¹⁰. In some embodiments, R³ is a 3-6 membered carbocycle or heterocycle that is unsubstituted or substituted with one or more R¹⁰. In some embodiments, R³ is a 3-7 membered carbocycle that is unsubstituted or substituted with one or more R¹⁰. In some embodiments, R³ is a 3-7 membered heterocycle that is unsubstituted or substituted with one or more R¹⁰.

In some embodiments, R³ is a carbocycle, which carbocycle is unsubstituted or substituted with one or more R¹⁰. In some embodiments, R³ is a multicyclic carbocycle. In some embodiments, R³ is a monocyclic carbocycle. In some embodiments, R³ is a carbocycle that is substituted with —N(R¹⁹)₂, wherein each R¹⁹ is independently selected from unsubstituted or substituted C₁₋₆ alkyl and H. In some embodiments, R³ is a carbocycle that is substituted with —NH₂.

In some embodiments, R³ is a heterocycle, which heterocycle is unsubstituted or substituted with one or more R¹⁰. In some embodiments, R³ is a multicyclic heterocycle. In some embodiments, R³ is a monocyclic heterocycle. In some embodiments, the heterocycle includes one or more nitrogen atoms. In some embodiments, R³ is a heterocycle that is substituted with —N(R¹⁹)₂, wherein each R¹⁹ is independently selected from unsubstituted or substituted C₁₋₆ alkyl and H. In some embodiments, R³ is a heterocycle that is substituted with —NH₂.

In some embodiments, R³ is selected from:

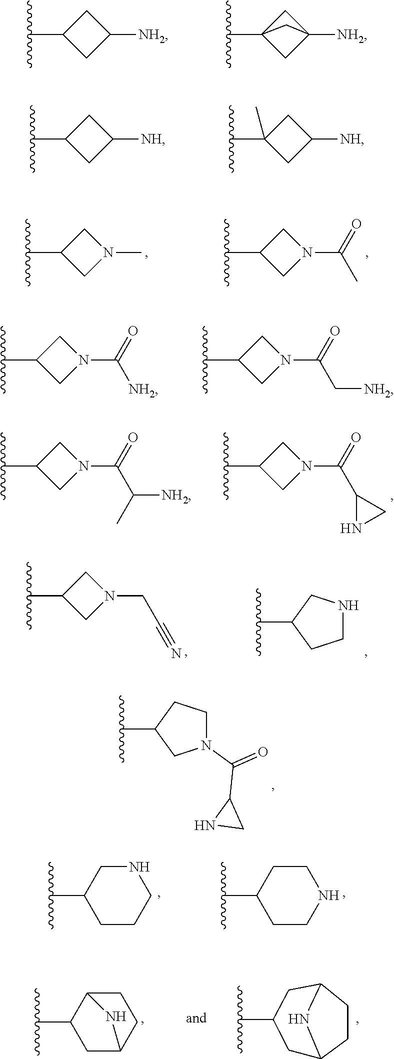

any of which is unsubstituted or substituted with one or more R¹⁰.

In some embodiments, R³ is selected from:

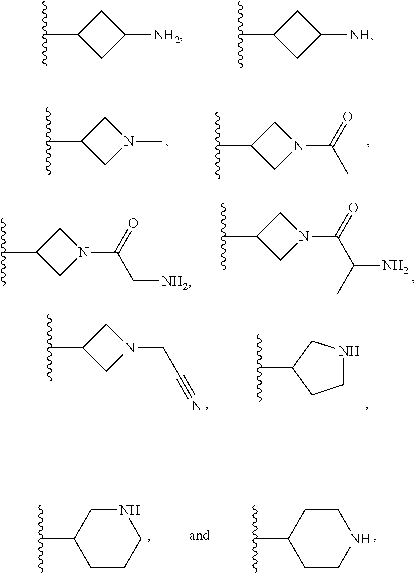

any of which is unsubstituted or substituted with one or more R¹⁰.

In some embodiments, R³ is selected from:

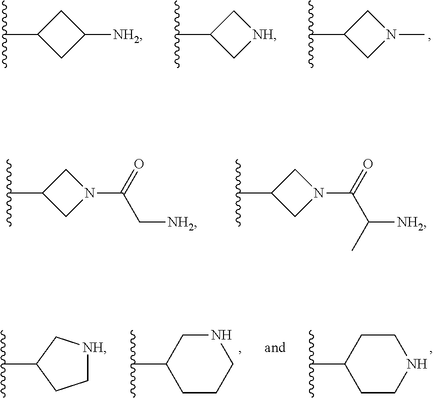

any of which is unsubstituted or substituted with one or more R¹⁰.

In some embodiments, R³ is selected from C₁₋₆ alkyl that is substituted with one or more R⁹. In some embodiments, R³ is selected from C₁₋₆alkyl-N(R¹⁷)₂, wherein each R¹⁷ is independently selected from C₁₋₆ alkyl and H. In some embodiments, R³ is C₁₋₆ alkyl substituted with —NH₂.

In some embodiments, R³ is selected from:

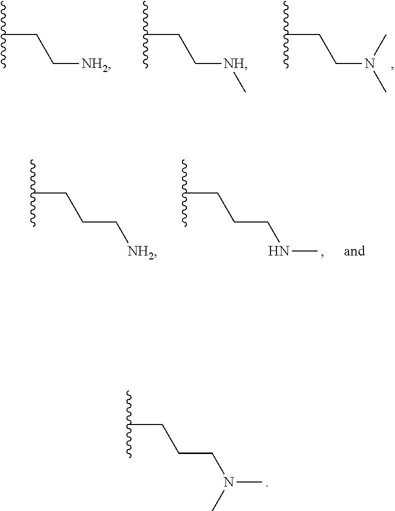

In some embodiments, R³ includes an amino moiety (e.g., —NRR′). In some embodiments, the amino moiety is a component of a heterocycle. In some embodiments, the amino moiety is appended to a carbocycle or heterocycle. In some embodiments, the amino moiety is a primary amine (e.g., —NH₂). In some embodiments, the amino moiety is a secondary amine (e.g., —NHR).

In some embodiments, R¹ is H.

In some embodiments, R¹ is —OR⁸. In some embodiments, R⁸ is a heterocycle. In some embodiments, R⁸ is an alkylheterocycle.

In some embodiments, R⁸ comprises a 3-6 membered heterocycle that is unsubstituted or substituted with one or more R¹⁶. In some embodiments, a heterocycle or a heterocycle of an alkylheterocycle comprises 4-8 members including at least one heteroatom selected from N, O, and S. In some embodiments, R⁸ comprises a heterocycle including at least one nitrogen atom. In some embodiments, the heterocycle comprises one or more R¹⁶ substituents. In some embodiments, at least one R¹⁶ is —OR¹², wherein R¹² is independently selected from C₁₋₆ alkyl, C₂₋₆ alkenyl, and H. In some embodiments, at least one R¹⁶ is —OCH₃.

In some embodiments, R¹ is selected from:

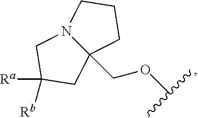

wherein R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R¹ is selected from:

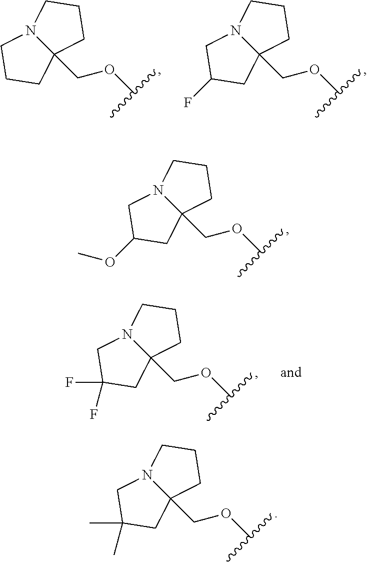 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, R¹ is selected from:

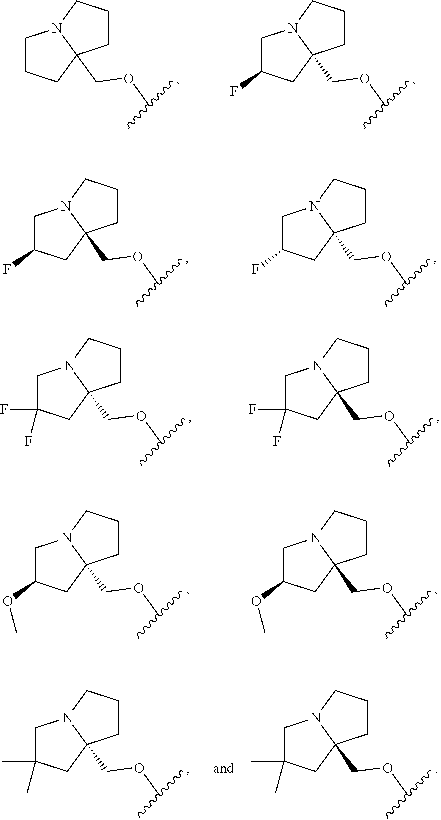

In some embodiments, R¹ is selected from:

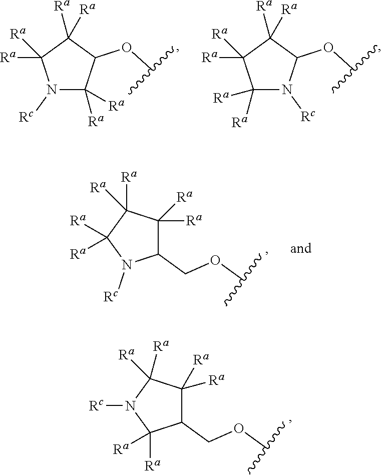

wherein each R^{a} is independently selected from halogen, C₁₋₆ alkyl, —OR¹², and H; and wherein R^{c} is selected from C₁₋₆ alkyl, wherein the C₁₋₆ alkyl is unsubstituted or is substituted with one or more R¹³. In some embodiments, R¹ is selected from:

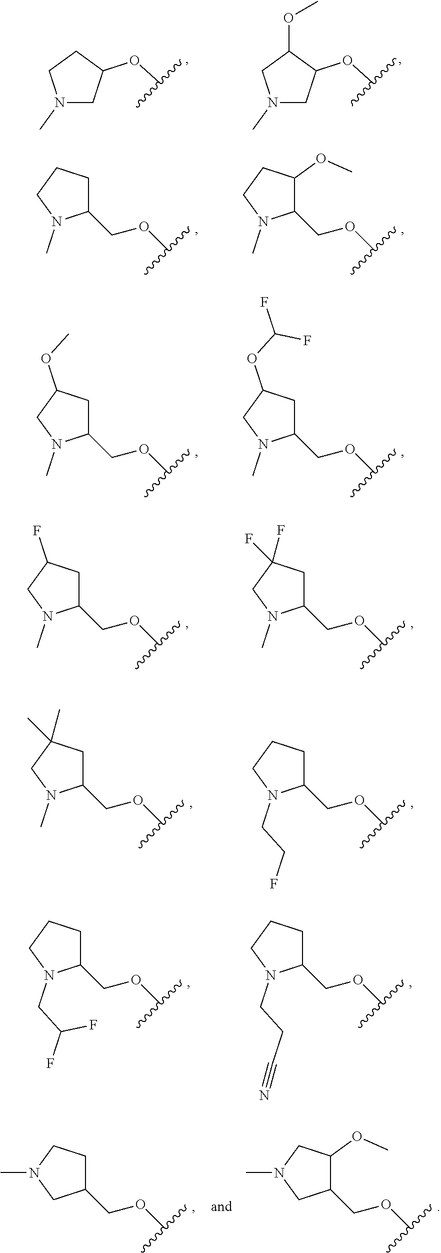

In some embodiments, R¹ is selected from:

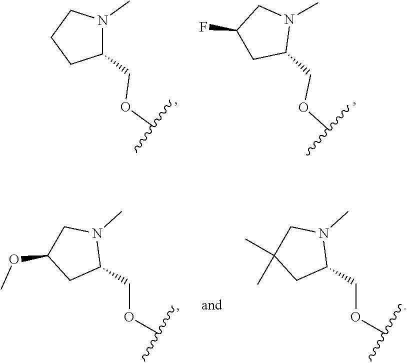

In some embodiments, R¹ is selected from:

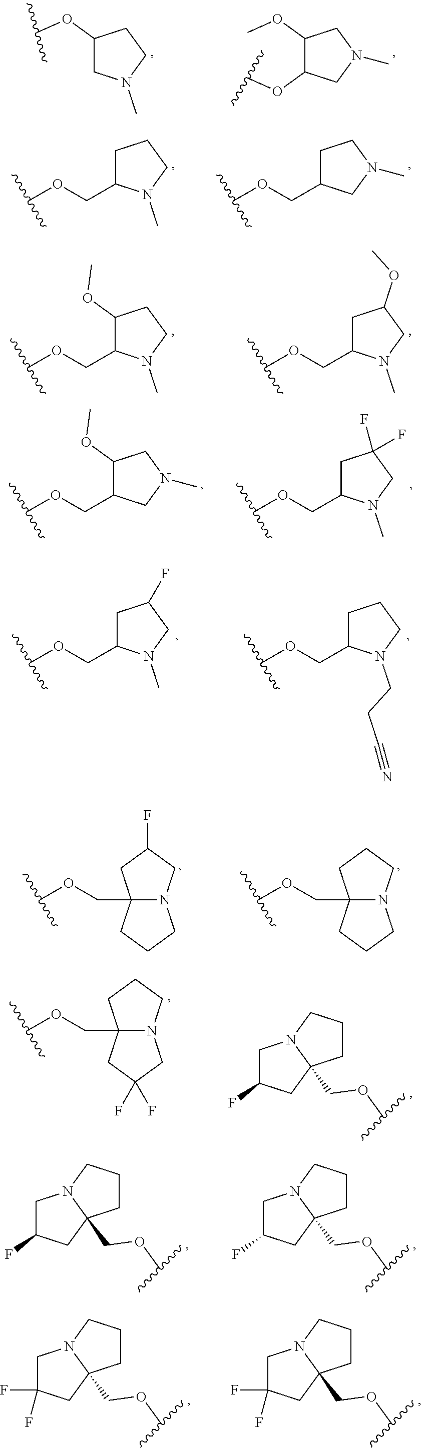

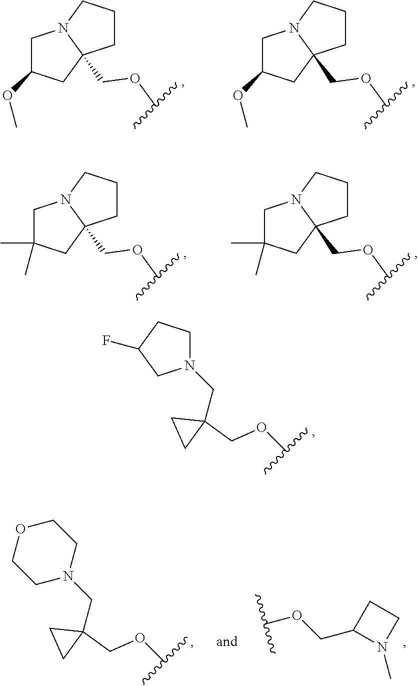

In some embodiments, R¹ is selected from:

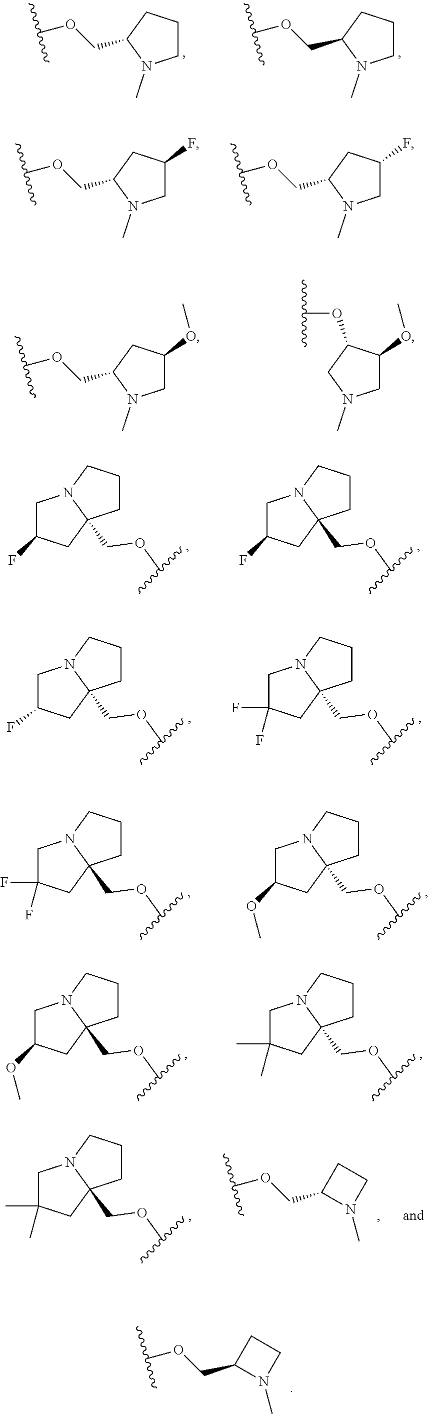

In some embodiments, R¹ is a 4-6 membered heterocycle including a nitrogen atom that is unsubstituted or substituted with one or more R¹⁶. In some embodiments, R¹ is selected from:

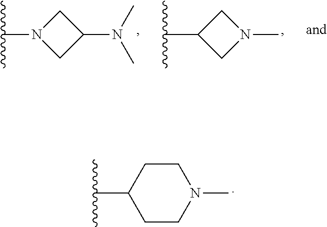

In some embodiments, R⁶ is selected from:

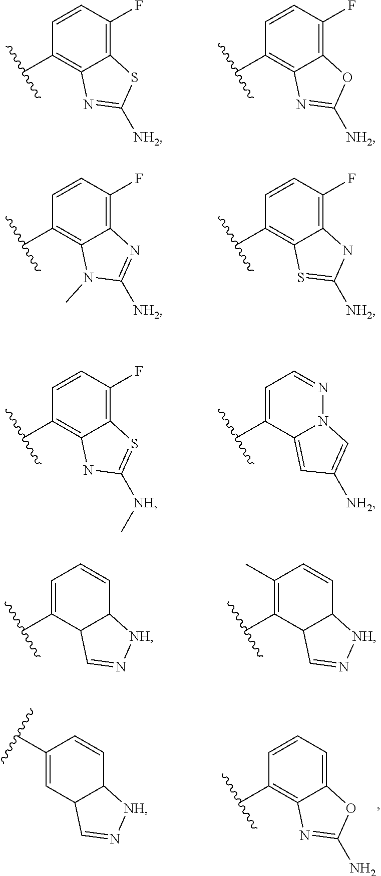

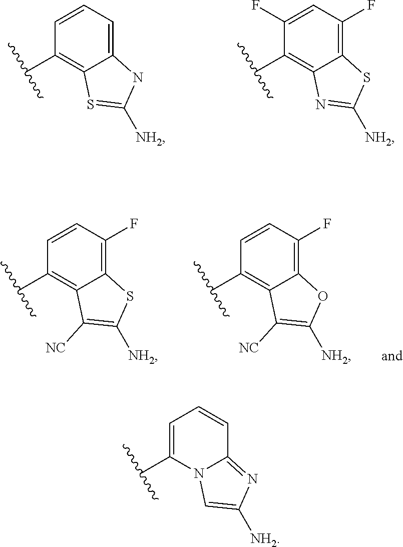

In some embodiments, R⁶ is selected from:

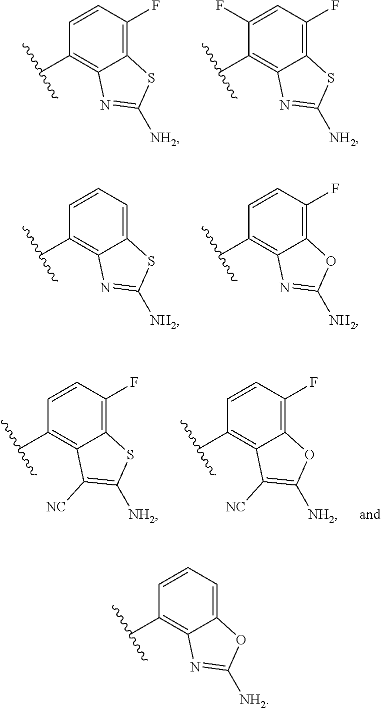

In some embodiments, R⁶ is:

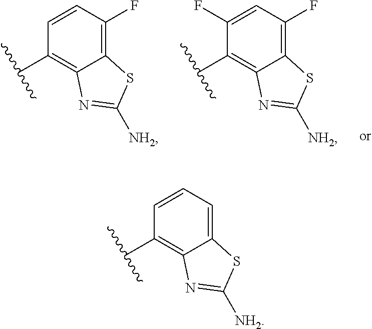 [SMILES: *c1ccc(F)c2sc(N)nc12 ; *c1c(F)cc(F)c2sc(N)nc12 ; "or" ; *c1cccc2sc(N)nc12]

In some embodiments, R⁴ is H. In some embodiments, R⁴ is a halogen.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, (i) R³ is a 4- or 5-membered heterocycle including a single nitrogen atom (e.g., an azetidine), which heterocycle is unsubstituted or substituted with one or more R¹⁰; and (ii) R¹ is —OR⁸ wherein R⁸ is a heterocycle or alkylheterocycle, wherein the heterocycle of the heterocycle or alkylheterocycle is a 4-8-membered ring system (e.g., 8-membered ring system or 5-membered ring system) comprising a single nitrogen atom. In some embodiments, (iii) R⁴ is H. In some embodiments, (iv) R⁷ is a halogen. In some embodiments, R³ is selected from:

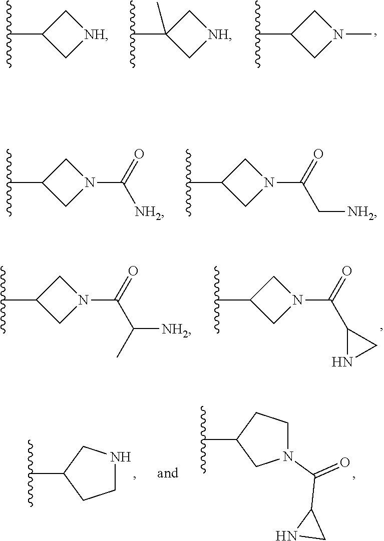

any of which is unsubstituted or substituted with one or more R¹⁰. In some embodiments, R¹ is selected from:

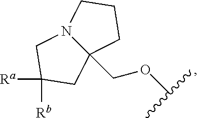

wherein each R^{a} and R^{b} are independently selected from halogen, C₁₋₆ alkyl, —OR¹² and H. In some embodiments, R¹ is selected from:

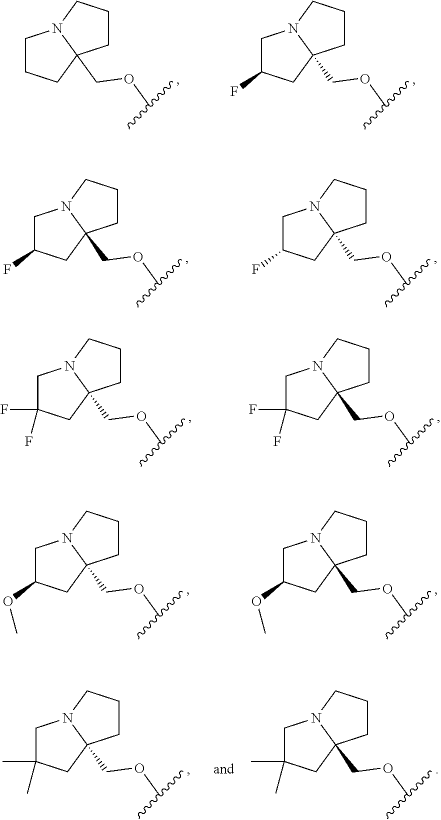

In some embodiments, R⁷ is F. In some embodiments, R⁶ is selected from:

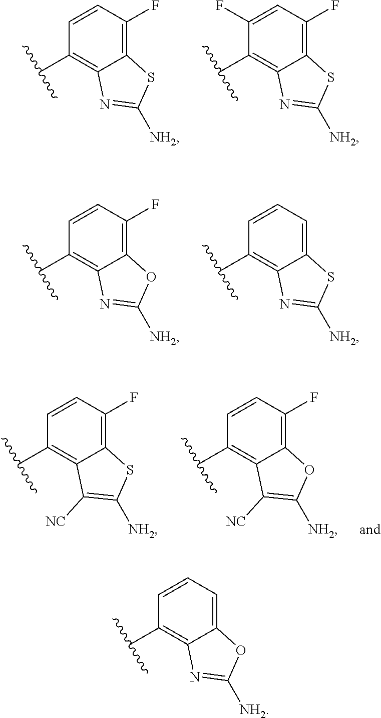

In some embodiments, R⁵ is selected from —CF₃, —Cl, —CH₂CN, furan, and phenyl. In some embodiments, (i) R³ is a 4-membered heterocycle including a single nitrogen atom (e.g., an azetidine), which heterocycle is unsubstituted or substituted with one or more R¹⁰; (ii) R¹ is —OR⁸ wherein R⁸ is an alkylheterocycle, wherein the heterocycle is an 8-membered ring system comprising a single nitrogen atom; (iii) R⁴ is H; and (iv) R⁷ is a halogen. In some embodiments, (i) R³ is a 5-membered heterocycle including a single nitrogen atom (e.g., an azetidine), which heterocycle is unsubstituted or substituted with one or more R¹⁰; (ii) R¹ is —OR⁸ wherein R⁸ is an alkylheterocycle, wherein the heterocycle is an 8-membered ring system comprising a single nitrogen atom; (iii) R⁴ is H; and (iv) R⁷ is a halogen.

In some embodiments, (i) R³ is a 4- or 5-membered heterocycle including a single nitrogen atom (e.g., an azetidine), which heterocycle is unsubstituted or substituted with one or more R¹⁰; and (ii) R¹ is —OR⁸ wherein R⁸ is a heterocycle or alkylheterocycle, wherein the heterocycle of the heterocycle or alkylheterocycle is a 4-8-membered ring system such as a 5-membered ring system comprising a single nitrogen atom. In some embodiments, (iii) R⁴ is H. In some embodiments, (iv) R⁷ is a halogen. In some embodiments, R³ is selected from:

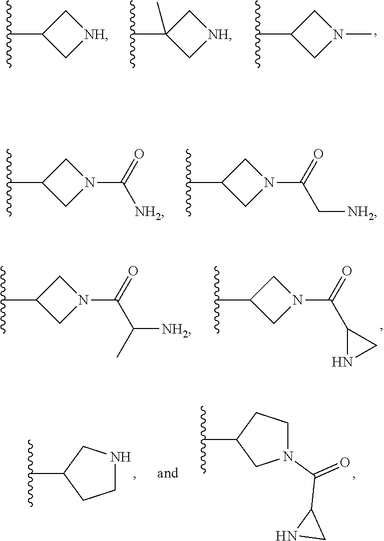

any of which is unsubstituted or substituted with one or more R¹⁰. In some embodiments, R¹ is selected from:

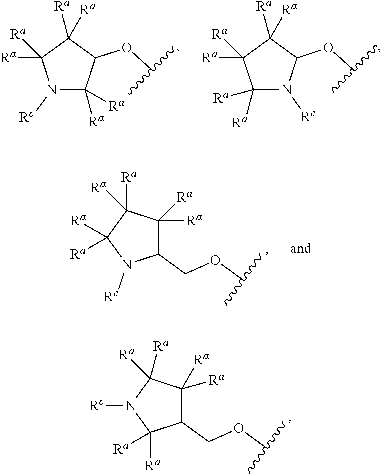

wherein each R^{a} is independently selected from halogen, C₁₋₆ alkyl, —OR¹², and H; and wherein R^{c} is selected from C₁₋₆ alkyl, wherein the C₁₋₆ alkyl is unsubstituted or is substituted with one or more R¹³. In some embodiments, R¹ is selected from:

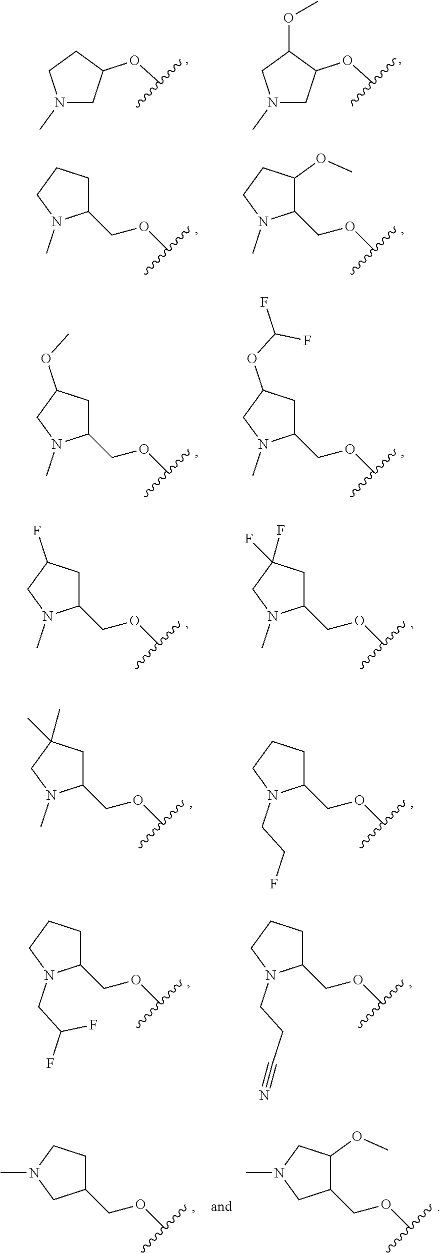

In some embodiments, R⁷ is F. In some embodiments, R⁶ is selected from:

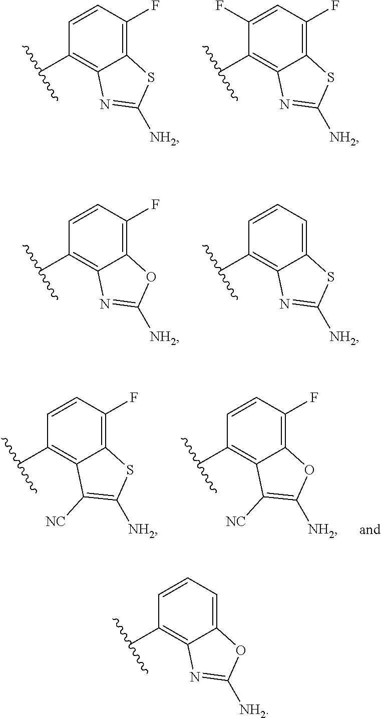

In some embodiments, R⁵ is selected from —CF₃, —Cl, furan, and phenyl. In some embodiments, (i) R³ is a 4-membered heterocycle including a single nitrogen atom (e.g., an azetidine), which heterocycle is unsubstituted or substituted with one or more R¹⁰; and (ii) R¹ is —OR⁸ wherein R⁸ is an alkylheterocycle, wherein the heterocycle is a 5-membered ring system comprising a single nitrogen atom; (iii) R⁴ is H; and (iv) R⁷ is a halogen. In some embodiments, (i) R³ is a 5-membered heterocycle including a single nitrogen atom (e.g., a pyrrolidine), which heterocycle is unsubstituted or substituted with one or more R¹⁰; and (ii) R¹ is —OR⁸ wherein R⁸ is an alkylheterocycle, wherein the heterocycle is a 5-membered ring system comprising a single nitrogen atom; (iii) R⁴ is H; and (iv) R⁷ is a halogen.

In some embodiments, (i) R³ is bridged carbocyclic or heterocyclic ring system that is unsubstituted or substituted with one or more R¹⁰; and (ii) R¹ is —OR⁸ wherein R⁸ is a heterocycle or alkylheterocycle, wherein the heterocycle of the heterocycle or alkylheterocycle is a 4-8-membered ring system comprising a single nitrogen atom. In some embodiments, (iii) R⁴ is H. In some embodiments, (iv) R⁷ is a halogen. In some embodiments, (i) R³ is bridged heterocyclic ring system that is unsubstituted or substituted with one or more R¹⁰; and (ii) R¹ is —OR⁸ wherein R⁸ is a heterocycle or alkylheterocycle, wherein the heterocycle of the heterocycle or alkylheterocycle is a 4-8-membered ring system comprising a single nitrogen atom. In some embodiments, (i) R³ is bridged carbocyclic ring system that is unsubstituted or substituted with one or more R¹⁰; and (ii) R¹ is —OR⁸ wherein R⁸ is a heterocycle or alkylheterocycle, wherein the heterocycle of the heterocycle or alkylheterocycle is a 4-8-membered ring system comprising a single nitrogen atom. In some embodiments, R³ is selected from:

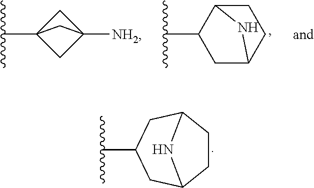 [SMILES: *C12CC(N)(C1)C2 ; *C1CC2CCC1N2 ; "," ; "and" ; *C1CC2CCC(C1)N2]

In some embodiments, R¹ is selected from:

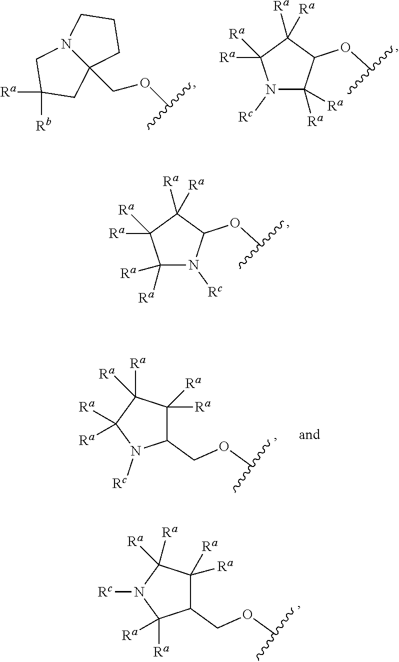

wherein each R^{a} and R^{b} are independently selected from halogen, C₁₋₆ alkyl, —OR¹², and H; and wherein R^{c} is selected from C₁₋₆ alkyl, wherein the C₁₋₆ alkyl is unsubstituted or is substituted with one or more R¹³. In some embodiments, R¹ is selected from:

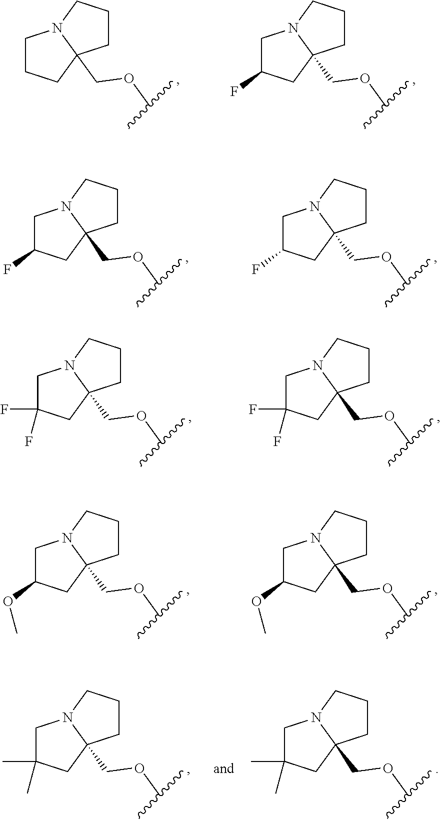

In some embodiments, R¹ is selected from:

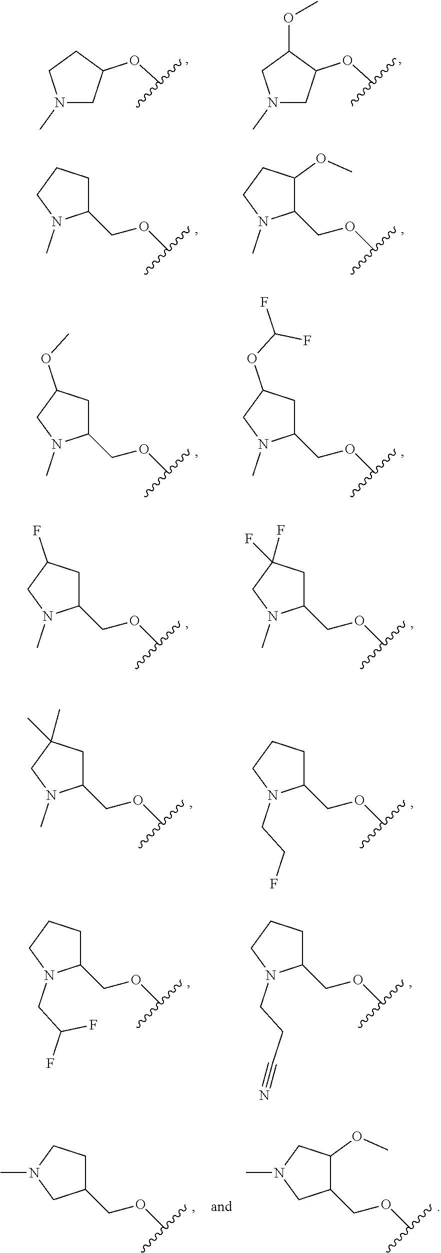

In some embodiments, R⁷ is F. In some embodiments, R⁵ is selected from —CF₃. In some embodiments, R⁶ is selected from:

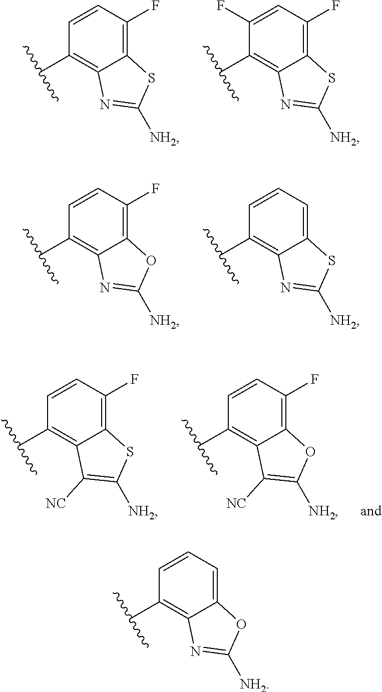

In some embodiments, (i) R³ is bridged heterocyclic ring system that is unsubstituted or substituted with one or more R¹⁰; (ii) R¹ is —OR⁸ wherein R⁸ is an alkylheterocycle, wherein the heterocycle is a 4-8-membered ring system comprising a single nitrogen atom; (iii) R⁴ is H; and (iv) R⁷ is a halogen.

In some embodiments, (i) R³ is selected from C₁₋₆alkyl-N(R¹⁷)₂; and (ii) R¹ is —OR⁸ wherein R⁸ is an alkylheterocycle, wherein the heterocycle is a 4-8-membered ring system comprising a single nitrogen atom. In some embodiments, (iii) R⁴ is H. In some embodiments, (iv) R⁷ is a halogen. In some embodiments, R³ is selected from:

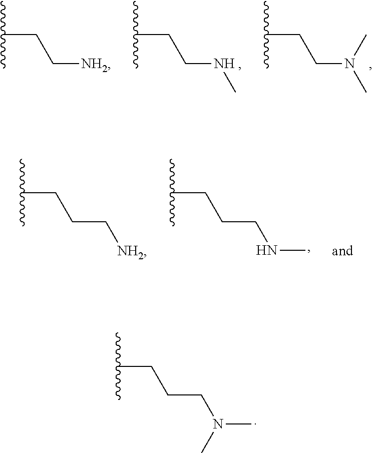

In some embodiments, (i) R³ is selected from C₂alkyl-N(R¹⁷)₂; (ii) R¹ is —OR⁸ wherein R⁸ is an alkylheterocycle, wherein the heterocycle is a 4-8-membered ring system comprising a single nitrogen atom; (iii) R⁴ is H; and (iv) R⁷ is a halogen. In some embodiments, R⁶ is:

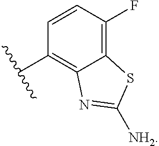 [SMILES: *c1ccc(F)c2sc(N)nc12]

In some embodiments, the present disclosure provides a compound according to Formula IA1:

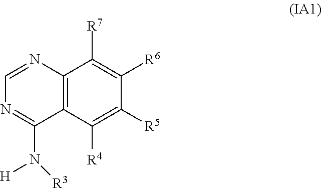

or a salt (e.g., pharmaceutically acceptable salt), ester, tautomer, prodrug, zwitterionic form, or stereoisomer thereof, wherein R³, R⁴, R⁵, R⁶, and R⁷ are as defined above for Formula I and described in classes and subclasses herein, both singly and in combination. In some embodiments, the present disclosure provides a compound of Formula IA1, or a salt (e.g., a pharmaceutically acceptable salt) thereof.

In some embodiments, the present disclosure provides a compound according to Formula IA1, wherein:
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹¹;
R⁷ is selected from halogen, —OR¹², —CN, and H;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen; each R¹⁴ is independently selected from halogen, —N(R¹²)₂, and C₁₋₆alkyl, wherein any C₁₋₆alkyl is unsubstituted or substituted with one or more R¹³;
each R¹⁵ is independently selected from halogen, —N(R¹²)₂, and C₁₋₆alkyl, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹; each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, R³ is selected from a carbocycle and a heterocycle, wherein the carbocycle or heterocycle is unsubstituted or substituted with one or more R¹⁰. In some embodiments, R³ is a 3-6 membered carbocycle or heterocycle that is unsubstituted or substituted with one or more R¹⁰. In some embodiments, R³ is a 3-7 membered carbocycle that is unsubstituted or substituted with one or more R¹⁰. In some embodiments, R³ is a 3-7 membered heterocycle that is unsubstituted or substituted with one or more R¹⁰.

In some embodiments, R³ is selected from:

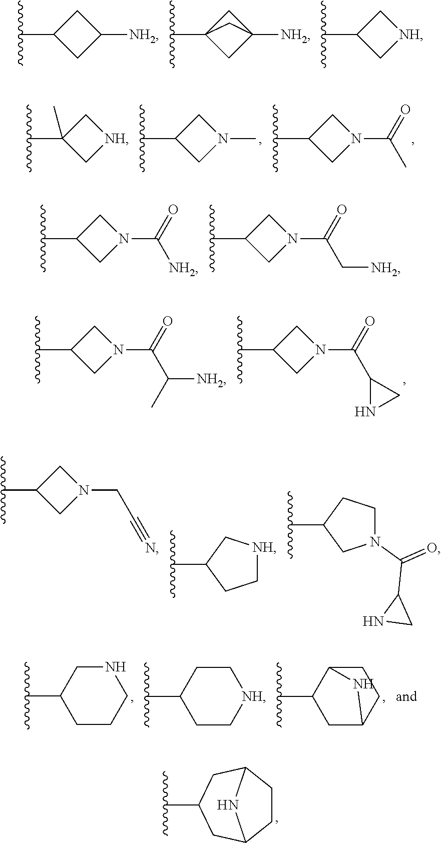

any of which is unsubstituted or substituted with one or more R¹⁰.

In some embodiments, R³ is selected from:

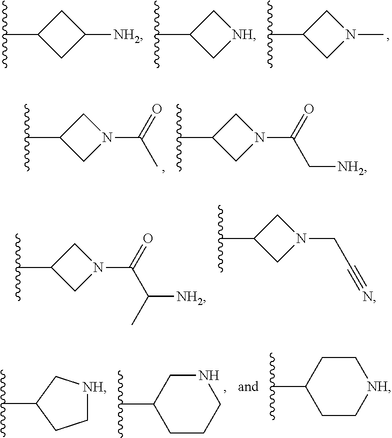

any of which is unsubstituted or substituted with one or more R¹⁰.

In some embodiments, R³ is selected from:

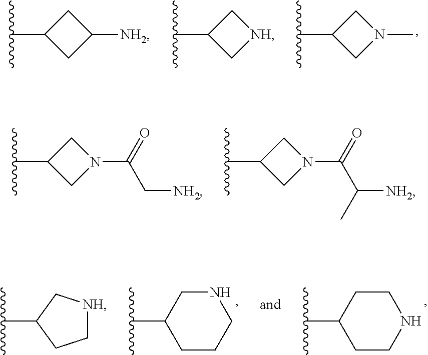

any of which is unsubstituted or substituted with one or more R¹⁰.

In some embodiments, R³ is selected from C₁₋₆ alkyl that is substituted with one or more R⁹. In some embodiments, R³ is selected from C₁₋₆alkyl-N(R¹⁷)₂, wherein each R⁷ is independently selected from C₁₋₆ alkyl and H. In some embodiments, R³ is C₁₋₆ alkyl substituted with —NH₂.

In some embodiments, R³ is selected from:

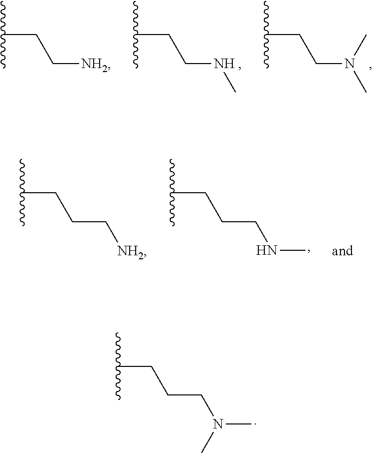

In some embodiments, R³ includes an amino moiety (e.g., —NRR′). In some embodiments, the amino moiety is a component of a heterocycle. In some embodiments, the amino moiety is appended to a carbocycle or heterocycle. In some embodiments, the amino moiety is a primary amine (e.g., —NH₂). In some embodiments, the amino moiety is a secondary amine (e.g., —NHR).

In some embodiments, R⁶ is selected from:

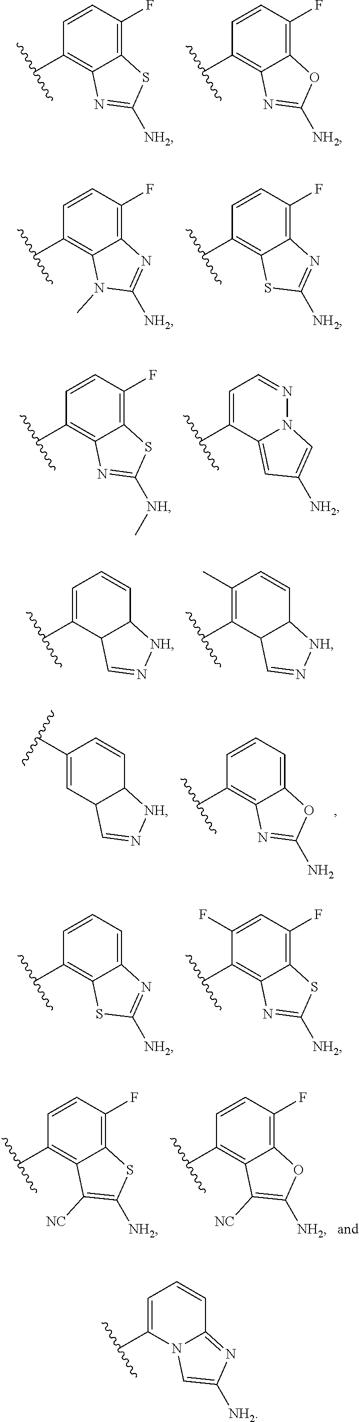

In some embodiments, R⁶ is selected from:

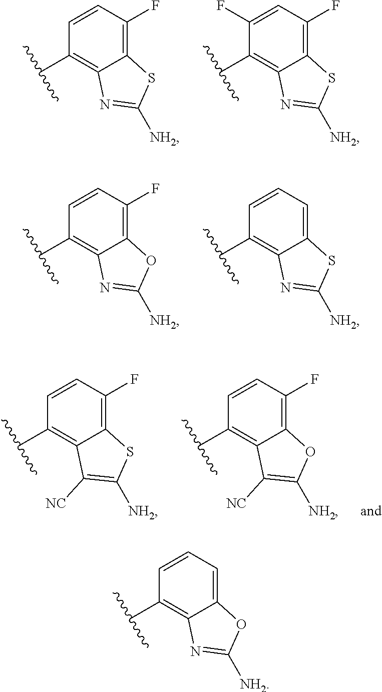

In some embodiments, R⁶ is:

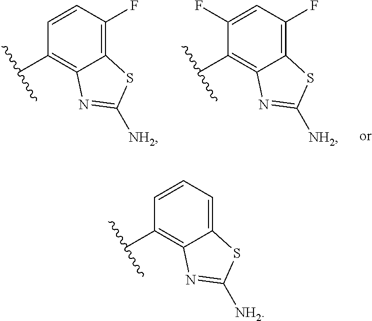 [SMILES: *c1ccc(F)c2sc(N)nc12 ; *c1c(F)cc(F)c2sc(N)nc12 ; "or" ; *c1cccc2sc(N)nc12]

In some embodiments, R⁴ is H. In some embodiments, R⁴ is a halogen.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, the present disclosure provides a compound according to Formula IA2:

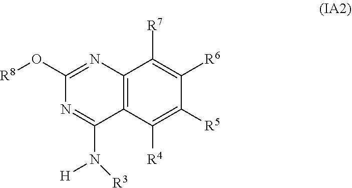

or a salt (e.g., pharmaceutically acceptable salt), ester, tautomer, prodrug, zwitterionic form, or stereoisomer thereof, wherein R³, R⁴, R⁵, R⁶, R⁷, and R⁸ are as defined above for Formula I and described in classes and subclasses herein, both singly and in combination. In some embodiments, the present disclosure provides a compound of Formula IA2, or a salt (e.g., a pharmaceutically acceptable salt) thereof.

In some embodiments, the present disclosure provides a compound of Formula IA2, wherein:
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H; R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, R⁸ comprises a 3-6 membered heterocycle that is unsubstituted or substituted with one or more R¹⁶.

In some embodiments, —OR⁸ is selected from:

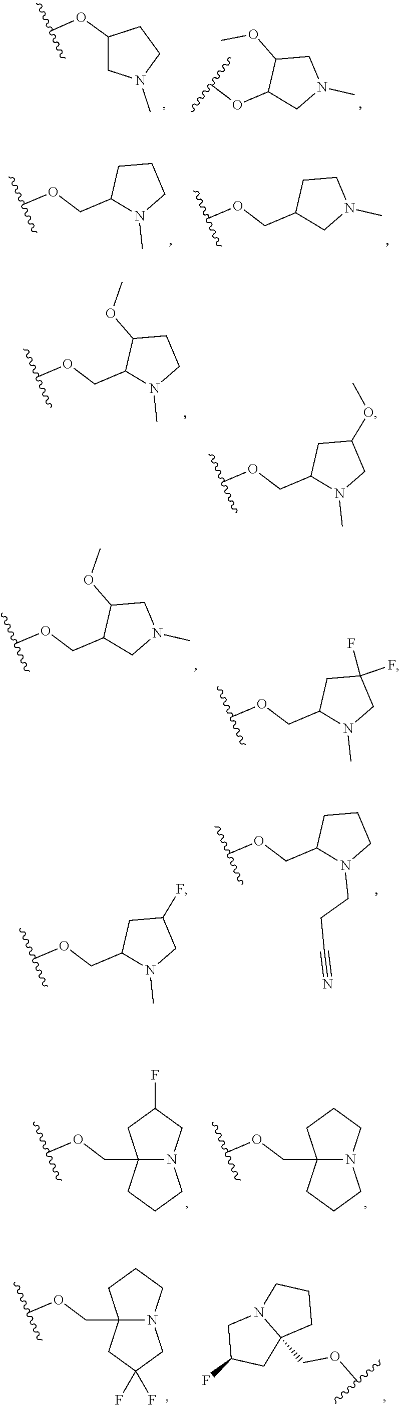

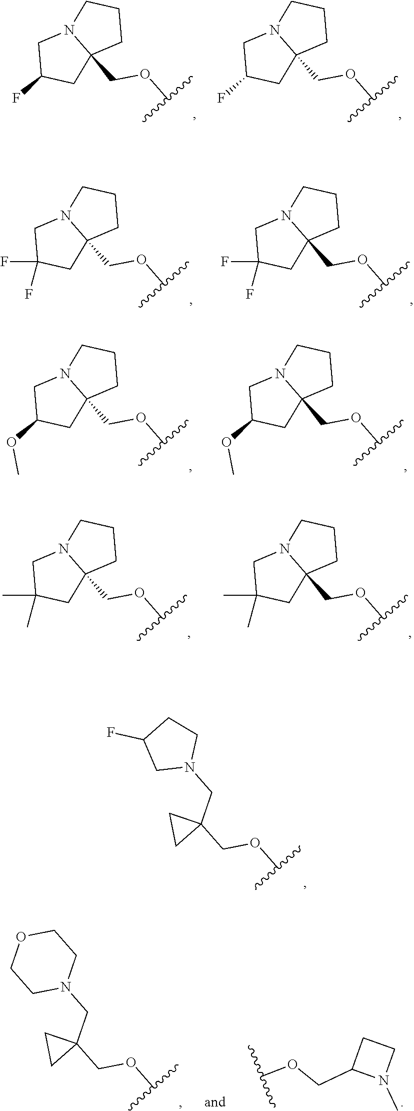

In some embodiments, —OR⁸ is selected from.

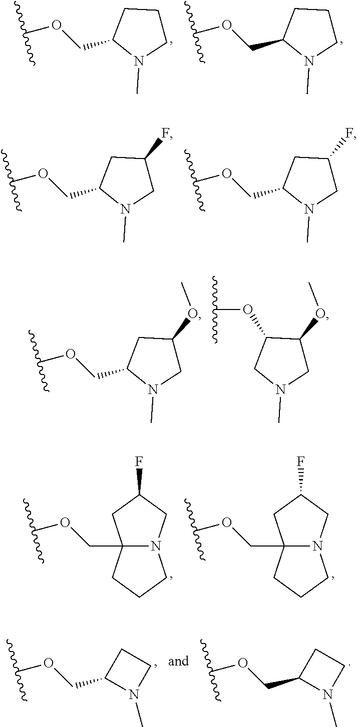

In some embodiments, R³ is selected from a carbocycle and a heterocycle, wherein the carbocycle or heterocycle is unsubstituted or substituted with one or more R¹⁰. In some embodiments, R³ is a 3-6 membered carbocycle or heterocycle that is unsubstituted or substituted with one or more R¹⁰. In some embodiments, R³ is selected from:

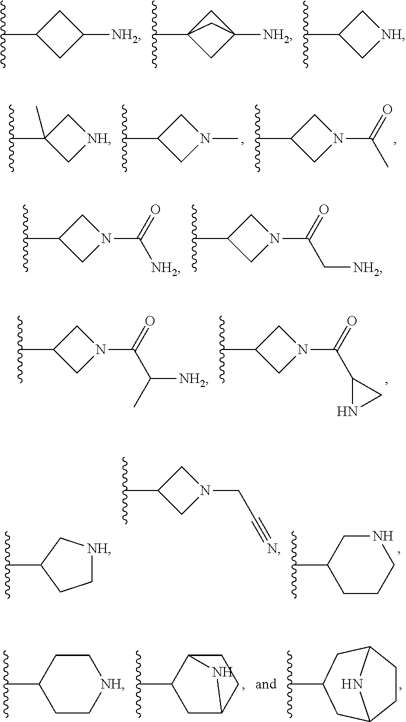

any of which is unsubstituted or substituted with one or more R¹⁰.

In some embodiments, R³ is selected from:

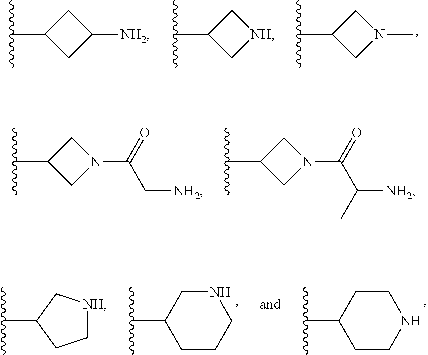

any of which is unsubstituted or substituted with one or more R¹⁰.

In some embodiments, R³ is selected from C₁₋₆ alkyl that is substituted with one or more R⁹. In some embodiments, R³ is selected from C₁₋₆alkyl-N(R¹⁷)₂. In some embodiments, R³ is selected from:

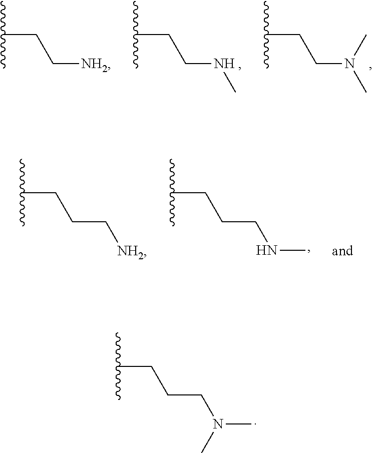

In some embodiments, R³ includes an amino moiety (e.g., —NRR′).

In some embodiments, R⁶ is selected from:

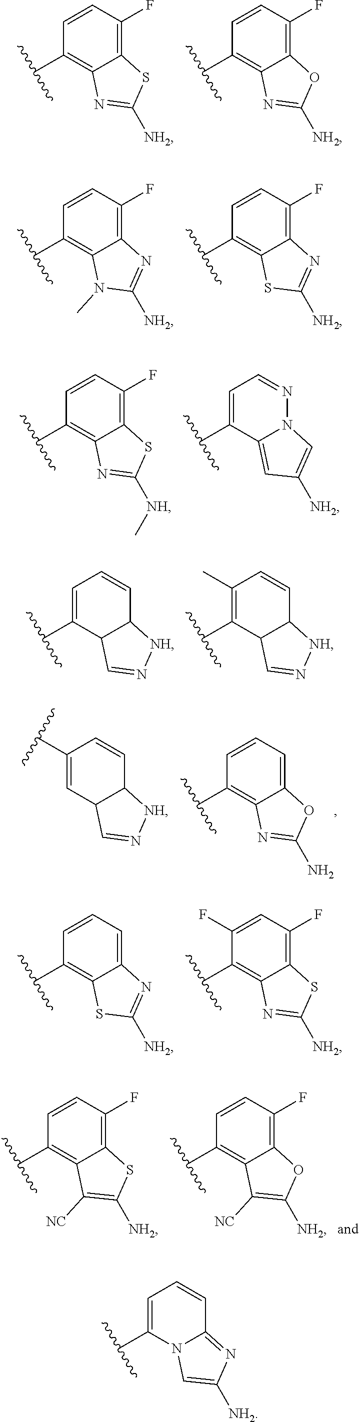

In some embodiments, R⁶ is selected from:

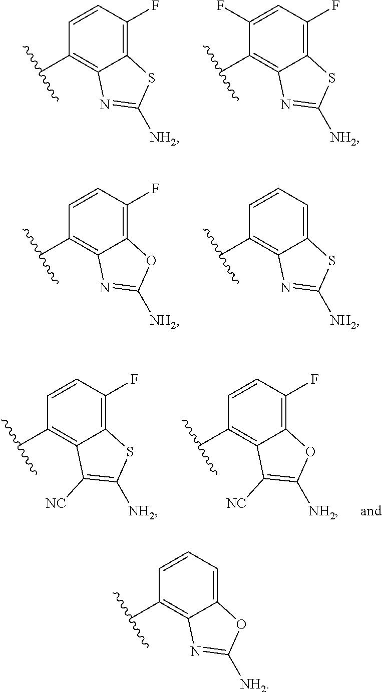

In some embodiments, R⁶ is:

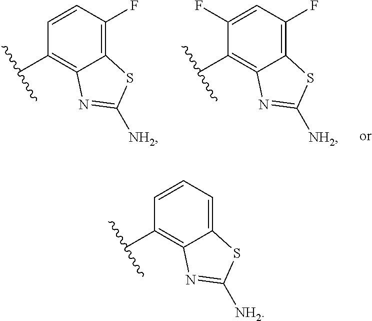 [SMILES: *c1ccc(F)c2sc(N)nc12 ; *c1c(F)cc(F)c2sc(N)nc12 ; "or" ; *c1cccc2sc(N)nc12]

In some embodiments, R⁴ is H. In some embodiments, R⁴ is a halogen.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In another aspect, the present disclosure provides a compound according to Formula IB:

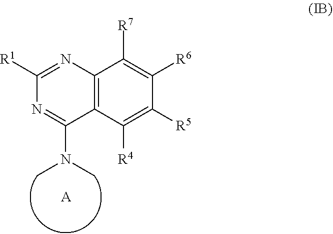

or a salt (e.g., pharmaceutically acceptable salt), ester, tautomer, prodrug, zwitterionic form, or stereoisomer thereof, wherein R¹, R⁴, R⁵, R⁶, and R⁷ are as defined above for Formula I and described in classes and subclasses herein, both singly and in combination; and Ring A is a heterocycle that is unsubstituted or substituted with one or more R¹¹. In some embodiments, the present disclosure provides a compound of Formula IB, or a salt (e.g., a pharmaceutically acceptable salt) thereof.

In some embodiments, the present disclosure provides a compound of Formula IB, wherein:
R¹ is selected from —OR⁸, a 4-6 membered heterocycle including a nitrogen atom, and H, wherein a heterocycle is unsubstituted or substituted with one or more R¹⁶;
Ring A is a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, Ring A is a monocyclic heterocycle that is unsubstituted or substituted with one or more R¹¹. In some embodiments, Ring A is a multicyclic heterocycle that is unsubstituted or substituted with one or more R¹¹. In some embodiments, Ring A is a 4-10 membered heterocycle that is unsubstituted or substituted with one or more R¹¹. In some embodiments, Ring A is a 4-6 membered heterocycle that is unsubstituted or substituted with one or more R¹¹. In some embodiments, Ring A includes at least one nitrogen atom. In some embodiments, Ring A has the structure:

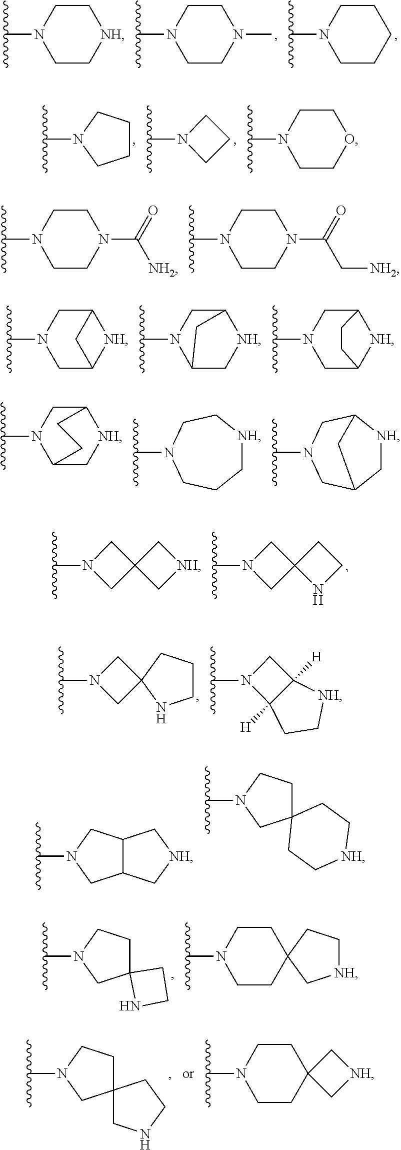

any of which is unsubstituted or substituted with one or more R¹¹. In some embodiments, Ring A has the structure:

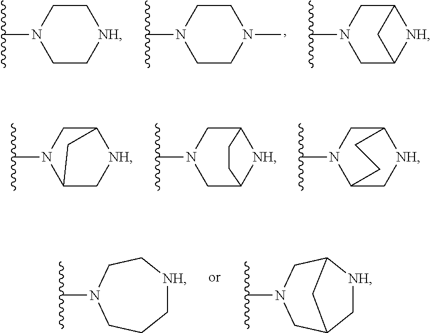

which ring is unsubstituted or substituted with one or more R¹¹. In some embodiments, Ring A has the structure:

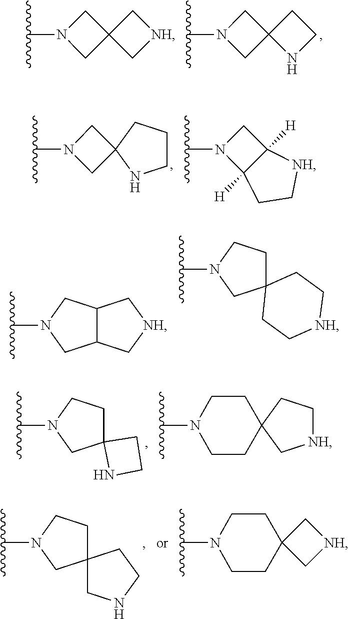

which ring is unsubstituted or substituted with one or more R¹¹. In some embodiments, Ring A has the structure:

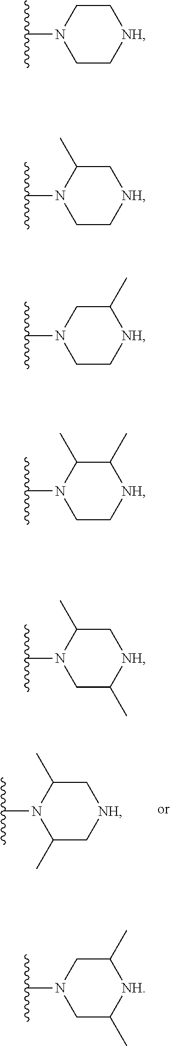

In some embodiments, Ring A has the structure:

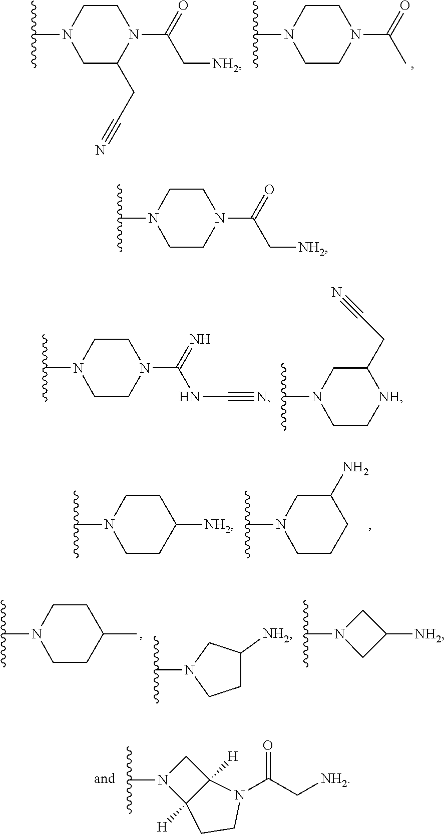

In some embodiments, Ring A is a 4-10 membered heterocycle that includes at least two nitrogen atoms (e.g., includes an amino moiety (e.g., —NR—)) and/or is substituted with a group including an amino moiety (e.g., —NRR′). In some embodiments, Ring A is a 4-6 membered heterocycle that includes at least two nitrogen atoms (e.g., includes an amino moiety (e.g., —NR—)) and/or is substituted with a group including an amino moiety (e.g., —NRR′).

In some embodiments, R¹ is H.

In some embodiments, R¹ is —OR⁸. In some embodiments, R⁸ is a heterocycle. In some embodiments, R⁸ is an alkylheterocycle. In some embodiments, a heterocycle or a heterocycle of an alkylheterocycle comprises 4-8 members including at least one heteroatom selected from N, O, and S. In some embodiments, R⁸ comprises a heterocycle including at least one nitrogen atom. In some embodiments, R⁸ comprises a 4-8 membered heterocycle including at least one nitrogen atom. In some embodiments, the heterocycle comprises one or more R¹⁶ substituents. In some embodiments, at least one R¹⁶ is —OR¹², wherein R¹² is independently selected from C₁₋₆ alkyl, C₂₋₆ alkenyl, and H. In some embodiments, at least one R¹⁶ is —OCH₃.

In some embodiments, R¹ is selected from:

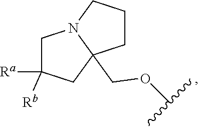

wherein R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R¹ is selected from:

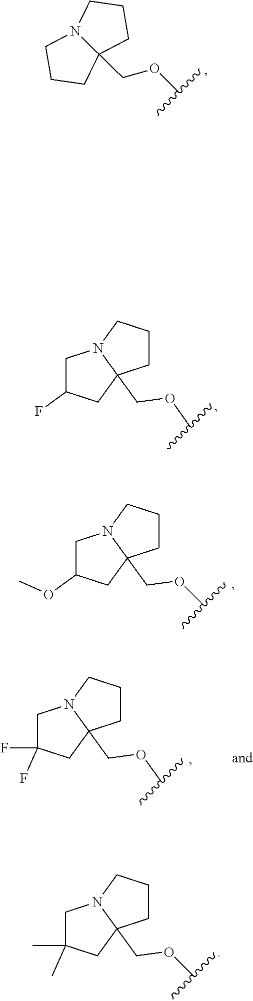

In some embodiments, R¹ is selected from:

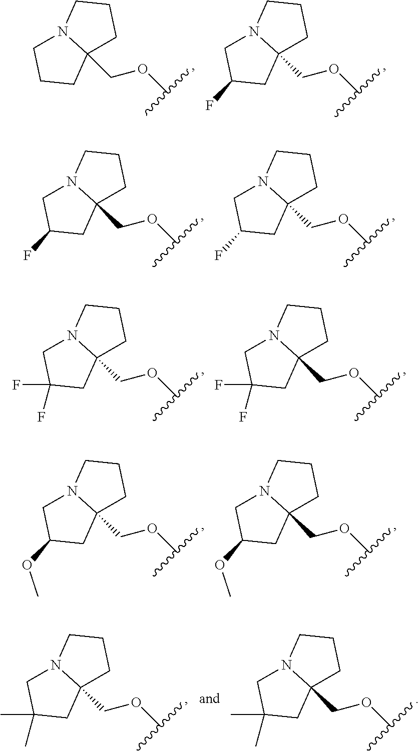

In some embodiments, R¹ is selected from:

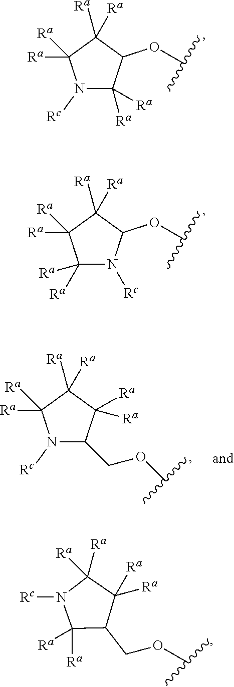

wherein each R^{a} is independently selected from halogen, C₁₋₆ alkyl, —OR¹², and H; and wherein R^{c} is selected from C₁₋₆ alkyl, wherein a C₁₋₆ alkyl of a R^{a} or R^{c} is unsubstituted or is substituted with one or more R¹³. In some embodiments R¹ is selected from:

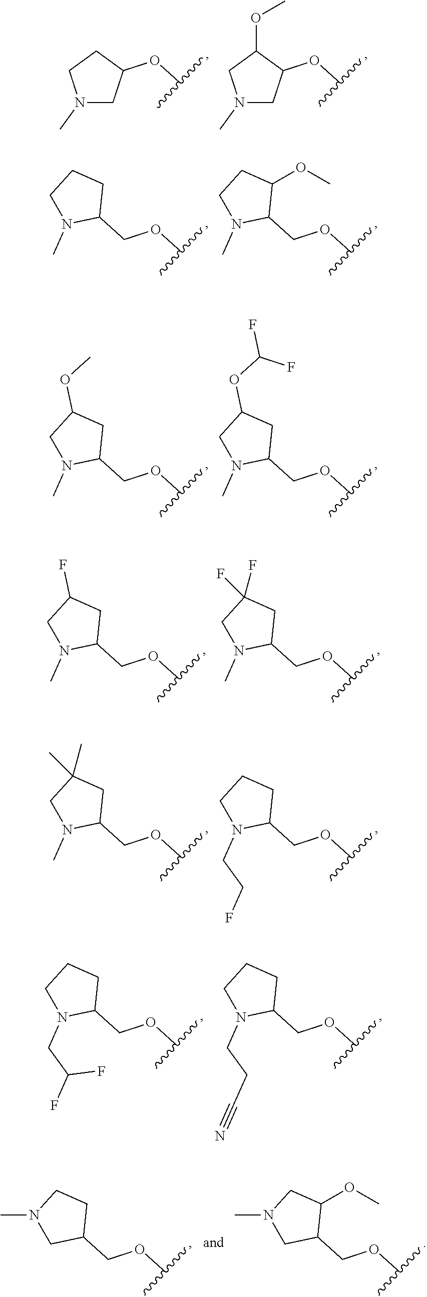

In some embodiments, R¹ is selected from:

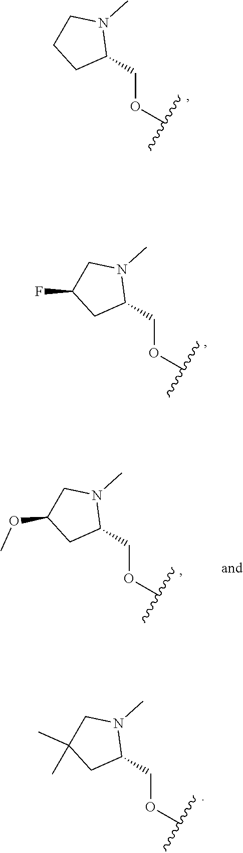

In some embodiments, R¹ is selected from:

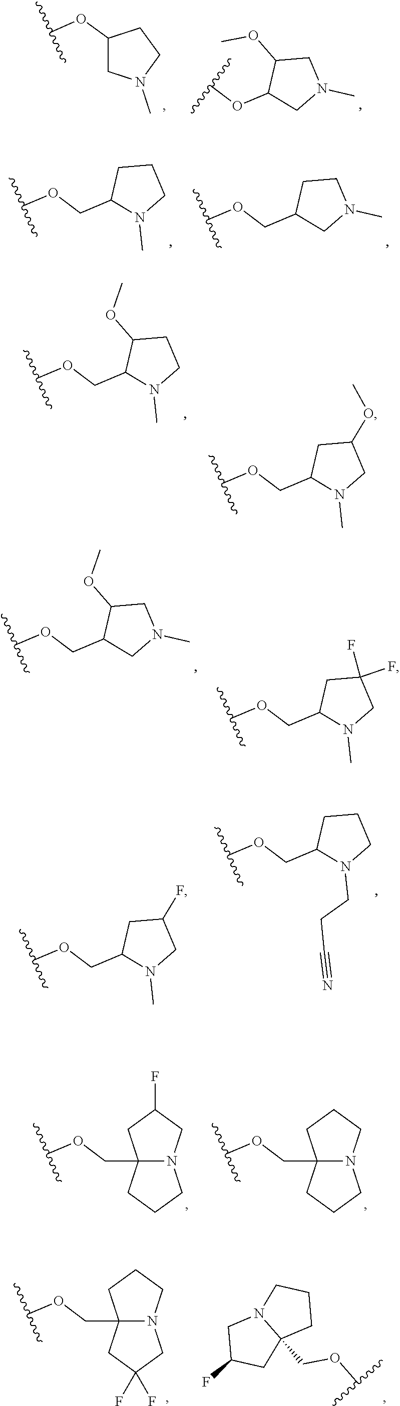

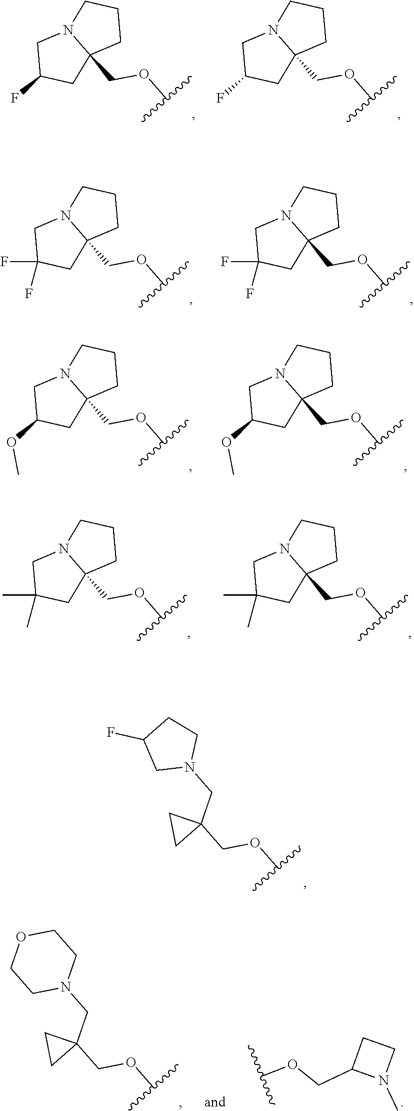

In some embodiments, R¹ is selected from:

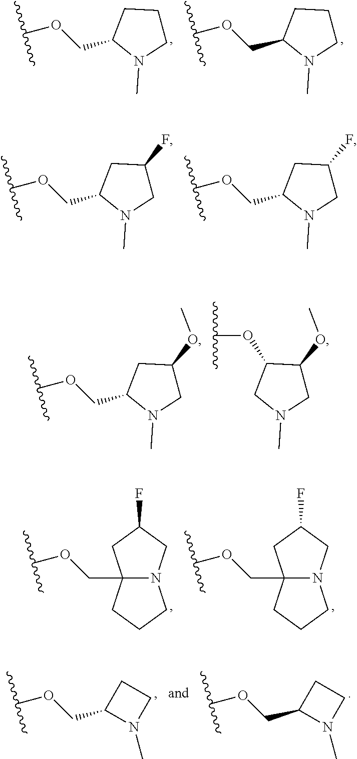

In some embodiments, R¹ is a 4-6 membered heterocycle including a nitrogen atom that is unsubstituted or substituted with one or more R¹⁶. In some embodiments, R¹ is selected from:

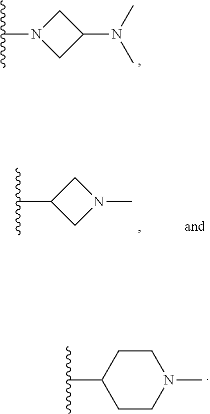

In some embodiments, R⁶ is selected from:

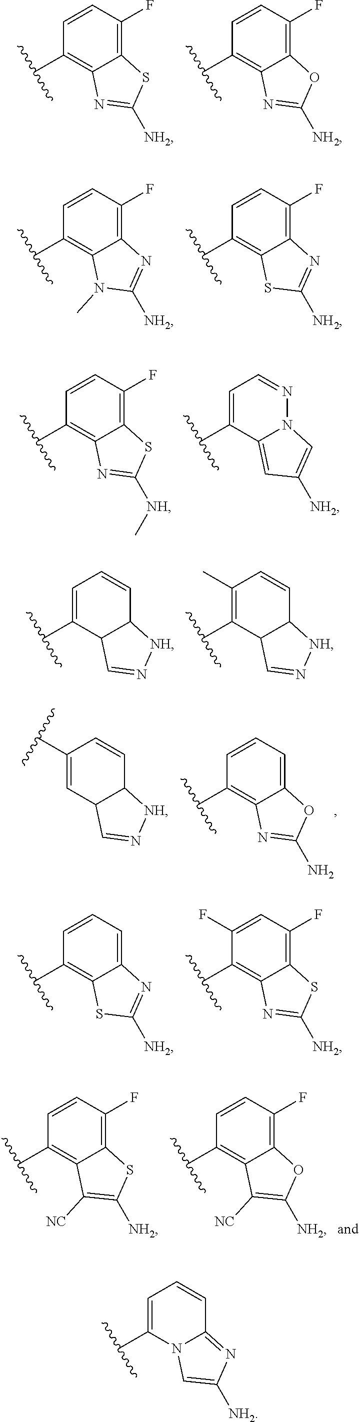

In some embodiments, R⁶ is selected from:

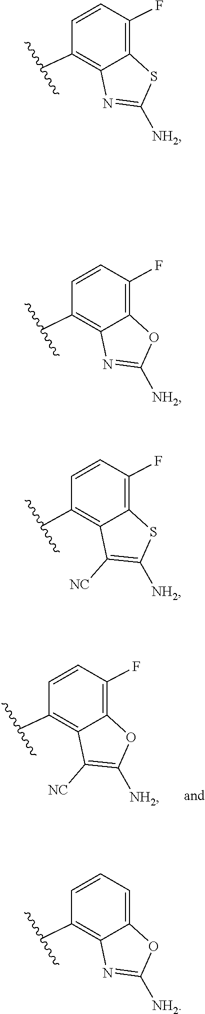 [SMILES: *c1ccc(F)c2sc(N)nc12 ; *c1ccc(F)c2oc(N)nc12 ; *c1ccc(F)c2sc(N)c(C#N)c12 ; *c1ccc(F)c2oc(N)c(C#N)c12 ; *c1cccc2oc(N)nc12]

In some embodiments R⁶ is.

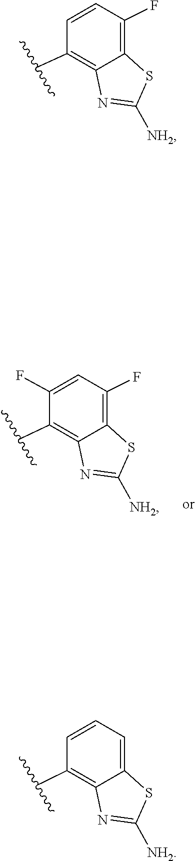 [SMILES: *c1ccc(F)c2sc(N)nc12 ; *c1c(F)cc(F)c2sc(N)nc12 ; *c1cccc2sc(N)nc12]

In some embodiments, R⁴ is H. In some embodiments, R⁴ is a halogen.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, the present disclosure provides a compound according to Formula IB1:

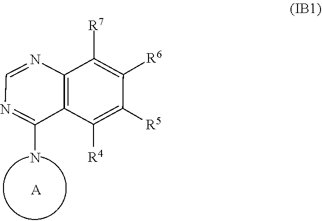

or a salt (e.g., pharmaceutically acceptable salt), ester, tautomer, prodrug, zwitterionic form, or stereoisomer thereof, wherein R⁴, R⁵, R⁶, and R⁷ are as defined above for Formula I and described in classes and subclasses herein, both singly and in combination; and Ring A is a heterocycle that is unsubstituted or substituted with one or more R¹¹. In some embodiments, the present disclosure provides a compound of Formula IB1, or a salt (e.g., a pharmaceutically acceptable salt) thereof.

In some embodiments, the present disclosure provides a compound of Formula IB1, wherein:
Ring A is a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, Ring A is a monocyclic heterocycle that is unsubstituted or substituted with one or more R¹¹. In some embodiments, Ring A is a multicyclic heterocycle that is unsubstituted or substituted with one or more R¹¹. In some embodiments, Ring A is a 4-10 membered heterocycle that is unsubstituted or substituted with one or more R¹¹. In some embodiments, Ring A is a 4-6 membered heterocycle that is unsubstituted or substituted with one or more R¹¹. In some embodiments, Ring A includes at least one nitrogen atom. In some embodiments, Ring A has the structure:

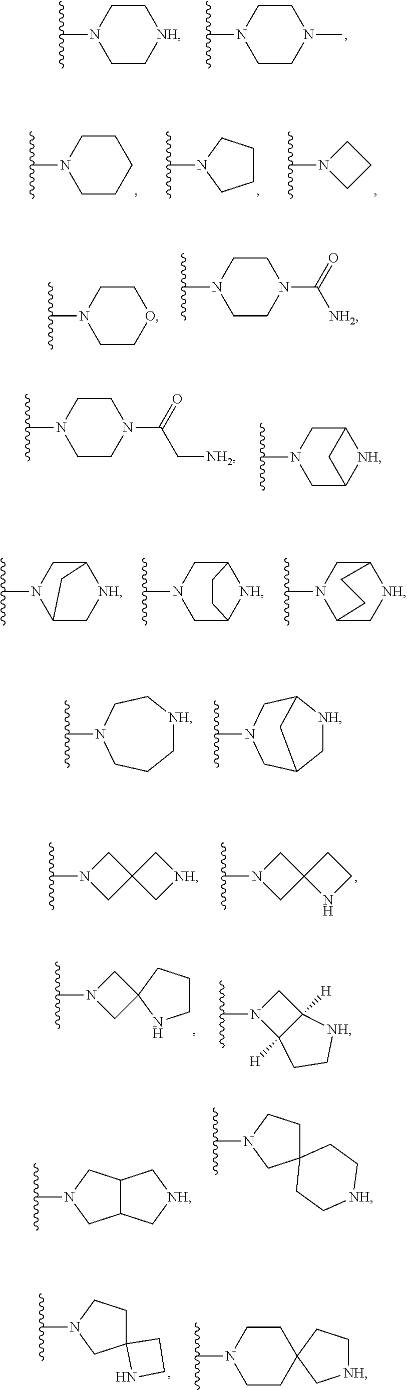

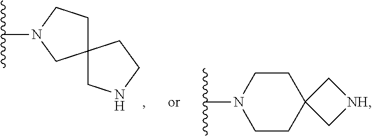 [SMILES: *N1CCC2(CCNC2)C1 ; *N1CCC2(CC1)CNC2]

any of which is unsubstituted or substituted with one or more R¹¹. In some embodiments, Ring A has the structure:

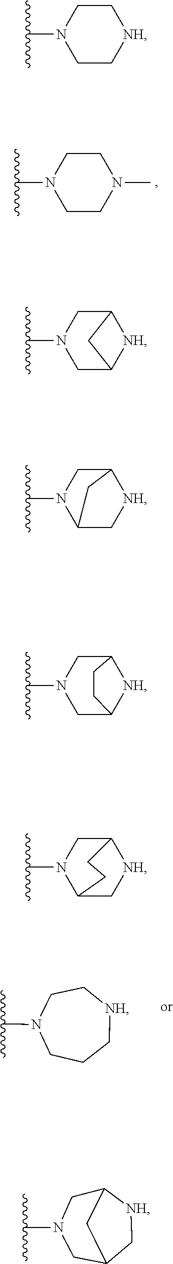

which ring is unsubstituted or substituted with one or more R¹¹. In some embodiments, Ring A has the structure:

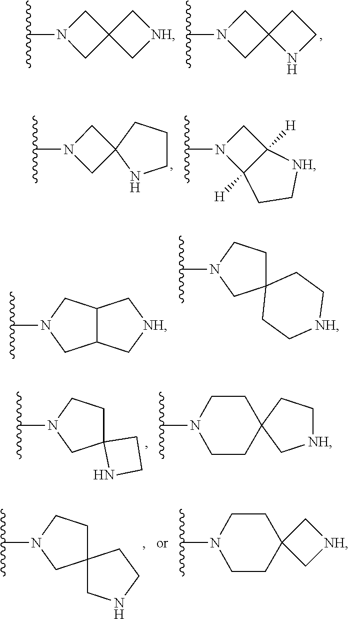

which ring is unsubstituted or substituted with one or more R¹¹. In some embodiments, Ring A has the structure:

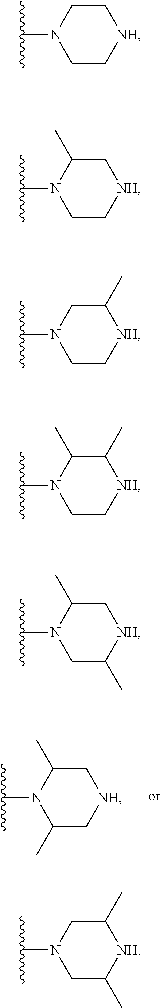

In some embodiments, Ring A has the structure:

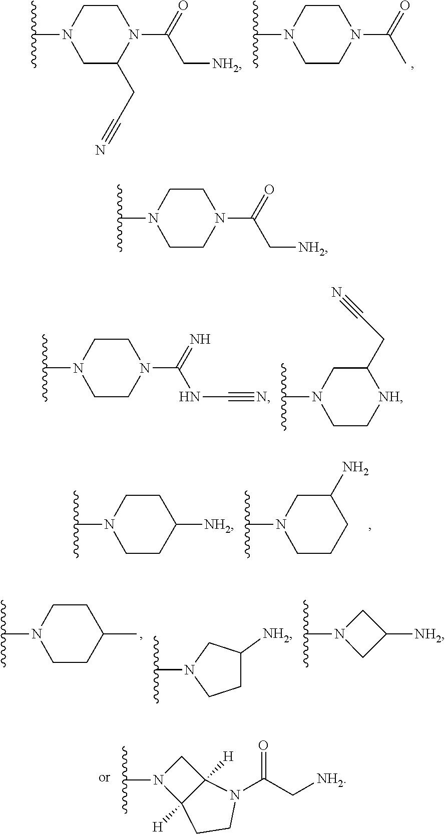

In some embodiments, Ring A is a 4-10 membered heterocycle that includes at least two nitrogen atoms (e.g., includes an amino moiety (e.g., —NR—)) and/or is substituted with a group including an amino moiety (e.g., —NRR′). In some embodiments, Ring A is a 4-6 membered heterocycle that includes at least two nitrogen atoms (e.g., includes an amino moiety (e.g., —NR—)) and/or is substituted with a group including an amino moiety (e.g., —NRR′).

In some embodiments, R⁶ is selected from:

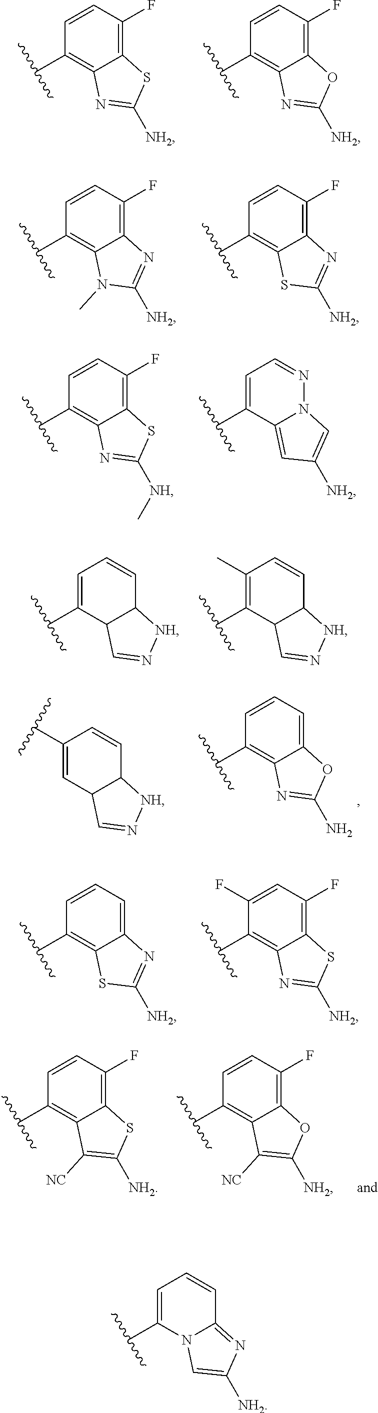

In some embodiments, R⁶ is selected from:

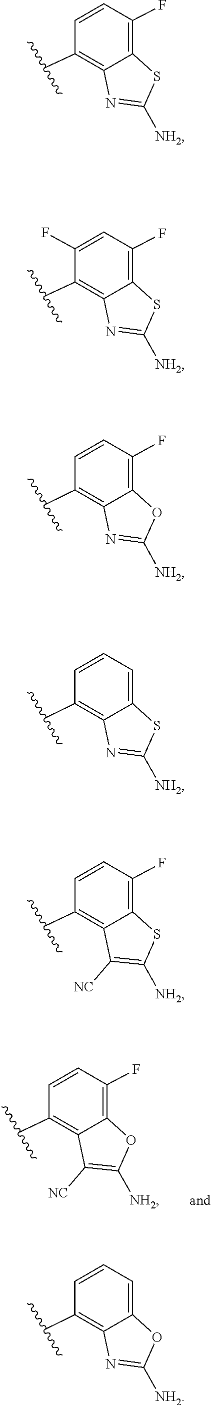

In some embodiments, R⁶ is:

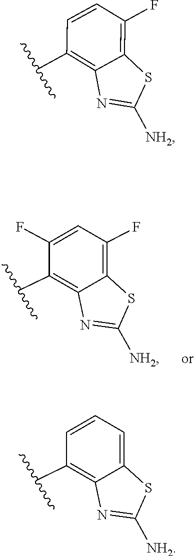 [SMILES: *c1ccc(F)c2sc(N)nc12 ; *c1c(F)cc(F)c2sc(N)nc12 ; *c1cccc2sc(N)nc12]

In some embodiments, R⁴ is H. In some embodiments, R⁴ is a halogen.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, (i) Ring A is a piperazine or diazepane that is unsubstituted or is substituted with one or more R¹¹. In some embodiments, (ii) R⁷ is a halogen. In some embodiments, (iii) R⁴ is H. In some embodiments, Ring A is selected from:

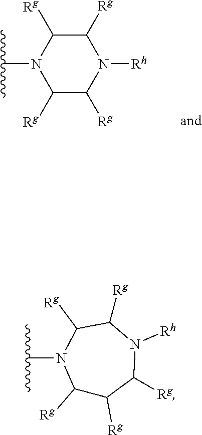

wherein each R^{g} is independently selected from H and C₁₋₆alkyl and R^{h} is selected from H, C₁₋₆alkyl, —C(O)NH₂, and —C(O)C₁₋₆alkylNH₂. In some embodiments, at least one R^{g} is C₁₋₆alkyl, such as C₁alkyl. In some embodiments, R^{h} is H. In some embodiments, Ring A is a piperazine or diazepane that is unsubstituted. In some embodiments, Ring A is selected from:

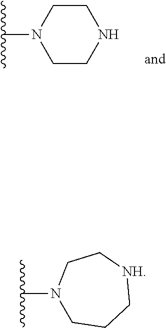 [SMILES: *N1CCNCC1 ; *N1CCCNCC1]

In some embodiments, R⁷ is F. In some embodiments, R⁵ is selected from a halogen, —CN, C₁₋₆alkyl, and 3-6 membered carbocycle, which carbocycle or C₁₋₆alkyl is unsubstituted or is substituted with one or more R¹³. In some embodiments, R⁶ is.

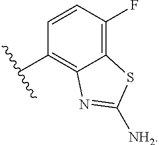 [SMILES: *c1ccc(F)c2sc(N)nc12]

In some embodiments, Ring A is a piperazine that is unsubstituted or is substituted with one or more R¹¹. In some embodiments, Ring A is a diazepane that is unsubstituted or is substituted with one or more R¹¹.

In some embodiments, (i) Ring A is a bridged heterocyclic ring system that is unsubstituted or is substituted with one or more R¹¹. In some embodiments, (ii) R⁷ is a halogen. In some embodiments, (iii) R⁴ is H. In some embodiments, Ring A is selected from:

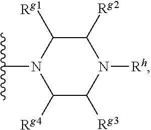

wherein (a) R^{g1} and R^{g3} or (b) R^{g2} and R^{g3} join together to form a second ring containing 4-6 members, and wherein any of R^{g1}, R^{g2}, R^{g3}, and R^{g4} that are not part of the second ring are independently selected from H and C₁₋₆alkyl; and R^{h} is selected from H, C₁₋₆alkyl, —C(O)NH₂, and —C(O)C₁₋₆alkylNH₂. In some embodiments, at least one of R^{g1}, R^{g2}, R^{g3}, and R^{g4} is C₁₋₆alkyl, such as C₁alkyl. In some embodiments, R^{h} is H. In some embodiments, Ring A is selected from:

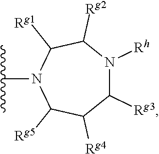

wherein R^{g2} and R^{g4} join together to form a second ring containing 4-6 members; R^{g1}, R^{g3}, and R^{g5} are independently selected from H and C₁₋₆alkyl; and R^{h} is selected from H, C₁₋₆alkyl, —C(O)NH₂, and —C(O)C₁₋₆alkylNH₂. In some embodiments, at least one of R^{g1}, R^{g3}, and R^{g5} is C₁₋₆alkyl, such as C₁alkyl. In some embodiments, R^{h} is H. In some embodiments, R^{h} is —C(O)NH₂. In some embodiments, Ring A is selected from:

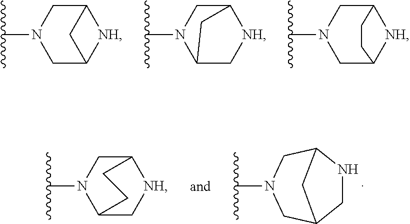

In some embodiments, R⁷ is F. In some embodiments, R⁵ is selected from a halogen, —CN, C₁₋₆alkyl, and 3-6 membered carbocycle, which carbocycle or C₁₋₆alkyl is unsubstituted or is substituted with one or more R¹³. In some embodiments, R⁶ is.

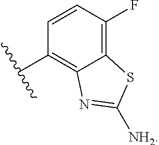 [SMILES: *c1ccc(F)c2sc(N)nc12]

In some embodiments, (i) Ring A is a bridged heterocyclic ring system that is unsubstituted or is substituted with one or more R¹¹; (ii) R⁷ is a halogen; and (iii) R⁴ is H.

In some embodiments, the present disclosure provides a compound according to Formula IB2:

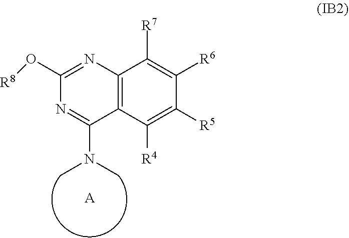

or a salt (e.g., pharmaceutically acceptable salt), ester, tautomer, prodrug, zwitterionic form, or stereoisomer thereof, wherein R⁴, R⁵, R⁶, R⁷, and R⁸ are as defined above for Formula I and described in classes and subclasses herein, both singly and in combination; and Ring A is a heterocycle that is unsubstituted or substituted with one or more R¹¹. In some embodiments, the present disclosure provides a compound of Formula IB2, or a salt (e.g., a pharmaceutically acceptable salt) thereof.

In some embodiments, the present disclosure provides a compound of Formula IB2, wherein:
Ring A is a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, Ring A is a monocyclic heterocycle that is unsubstituted or substituted with one or more R¹¹. In some embodiments, Ring A is a multicyclic heterocycle that is unsubstituted or substituted with one or more R¹¹. In some embodiments, Ring A is a 4-10 membered heterocycle that is unsubstituted or substituted with one or more R¹¹. In some embodiments, Ring A is a 4-6 membered heterocycle that is unsubstituted or substituted with one or more R¹¹. In some embodiments, Ring A includes at least one nitrogen atom. In some embodiments, Ring A has the structure:

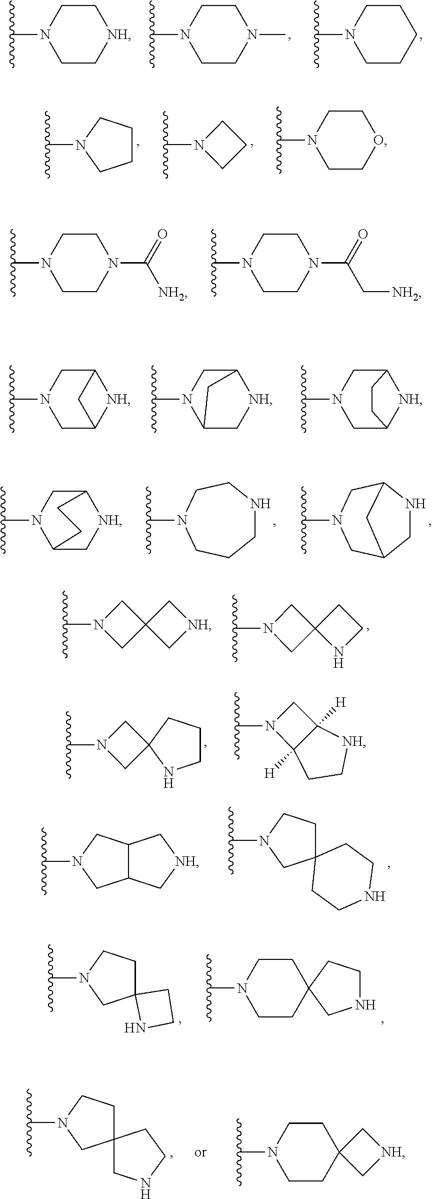

any of which is unsubstituted or substituted with one or more R¹¹. In some embodiments, Ring A has the structure:

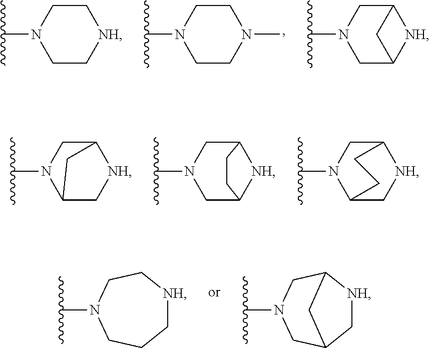

which ring is unsubstituted or substituted with one or more R¹¹. In some embodiments, Ring A has the structure:

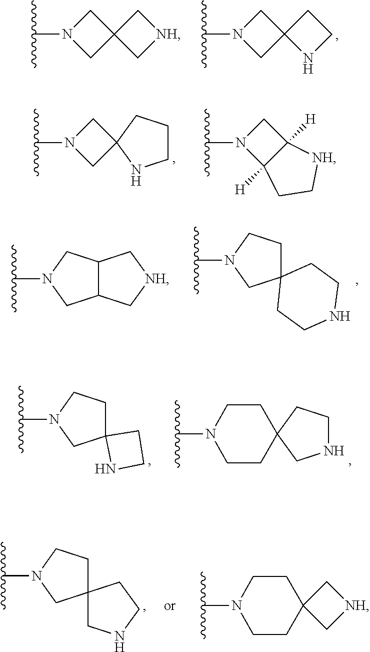

which ring is unsubstituted or substituted with one or more R¹¹. In some embodiments, Ring A has the structure:

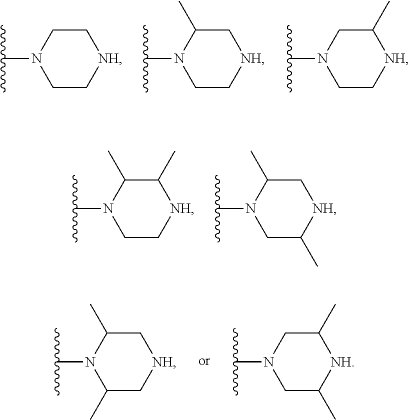

In some embodiments, Ring A has the structure:

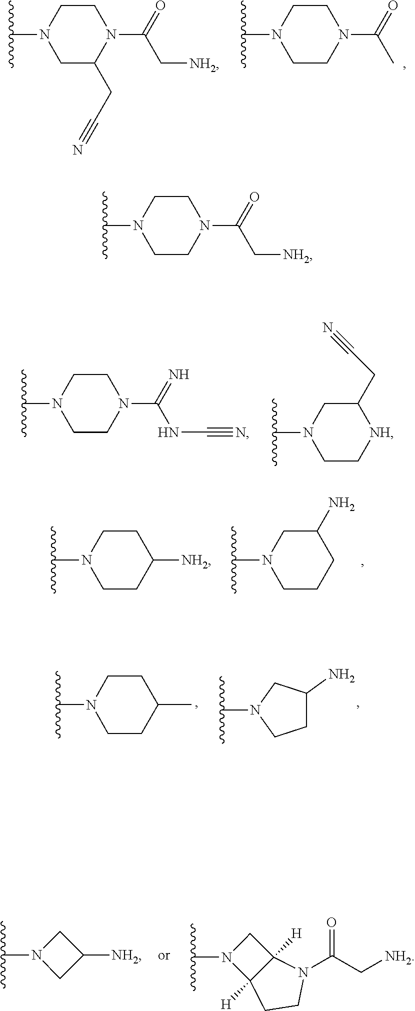

In some embodiments, Ring A is a 4-10 membered heterocycle that includes at least two nitrogen atoms (e.g., includes an amino moiety (e.g., —NR—)) and/or is substituted with a group including an amino moiety (e.g., —NRR′). In some embodiments, Ring A is a 4-6 membered heterocycle that includes at least two nitrogen atoms (e.g., includes an amino moiety (e.g., —NR—)) and/or is substituted with a group including an amino moiety (e.g., —NRR′).

In some embodiments, R⁸ is a heterocycle. In some embodiments, R⁸ is an alkylheterocycle. In some embodiments, a heterocycle or a heterocycle of an alkylheterocycle comprises 4-8 members including at least one heteroatom selected from N, O, and S. In some embodiments, R⁸ comprises a heterocycle including at least one nitrogen atom. In some embodiments, R⁸ comprises a 4-8 membered heterocycle including at least one nitrogen atom. In some embodiments, R⁸ comprises a 3-6 membered heterocycle that is unsubstituted or substituted with one or more R¹⁶. In some embodiments, the heterocycle comprises one or more R¹⁶ substituents. In some embodiments, at least one R¹⁶ is —OR¹², wherein R¹² is independently selected from C₁₋₆ alkyl, C₂₋₆ alkenyl, and H. In some embodiments, at least one R¹⁶ is —OCH₃.

In some embodiments, —OR⁸ is selected from:

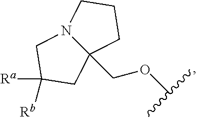

wherein R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, —OR⁸ is selected from:

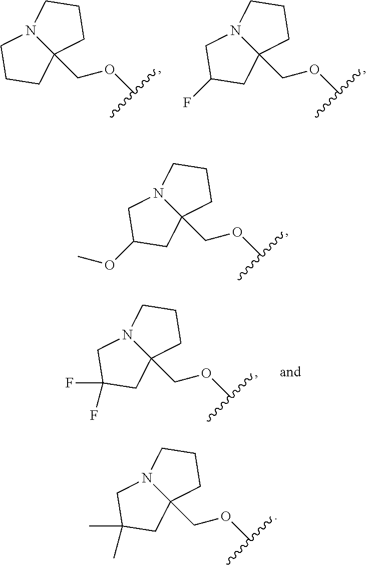 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, —OR⁸ is selected from:

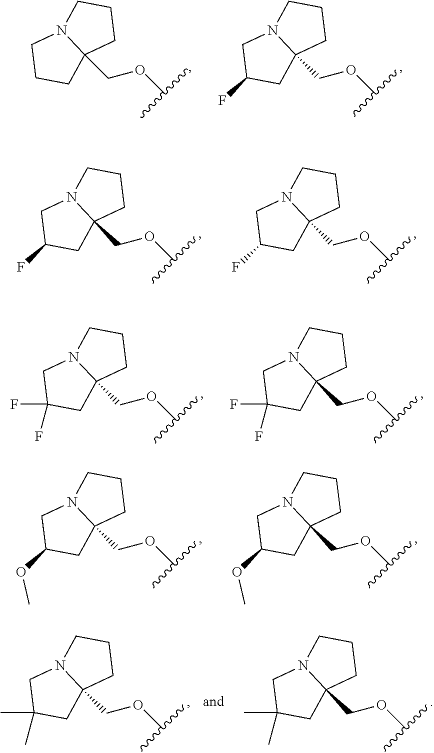

In some embodiments, —OR⁸ is selected from:

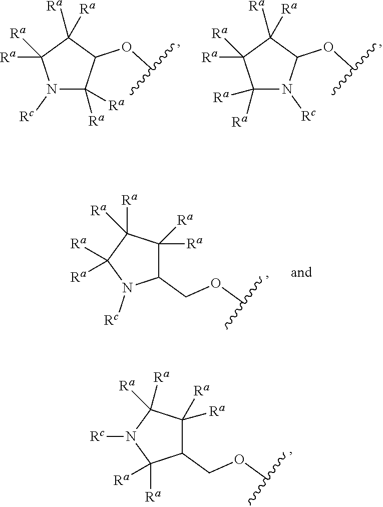

wherein each R^{a} is independently selected from halogen, C₁₋₆ alkyl, —OR¹², and H; and wherein R^{c} is selected from C₁₋₆ alkyl, wherein a C₁₋₆ alkyl of a R^{a} or R^{c} is unsubstituted or is substituted with one or more R¹³. In some embodiments, —OR⁸ is selected from:

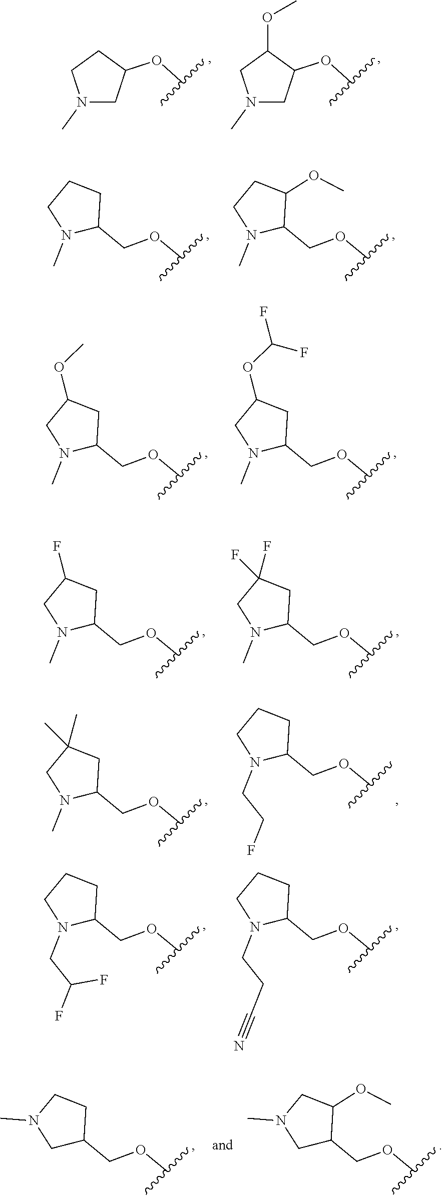

In some embodiments, —OR⁸ is selected from:

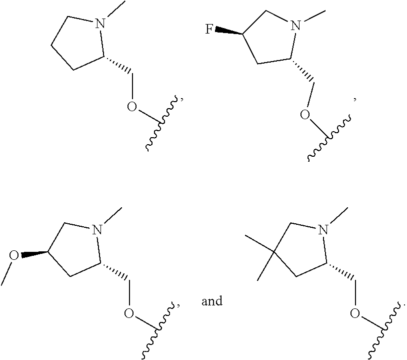

In some embodiments, —OR⁸ is selected from:

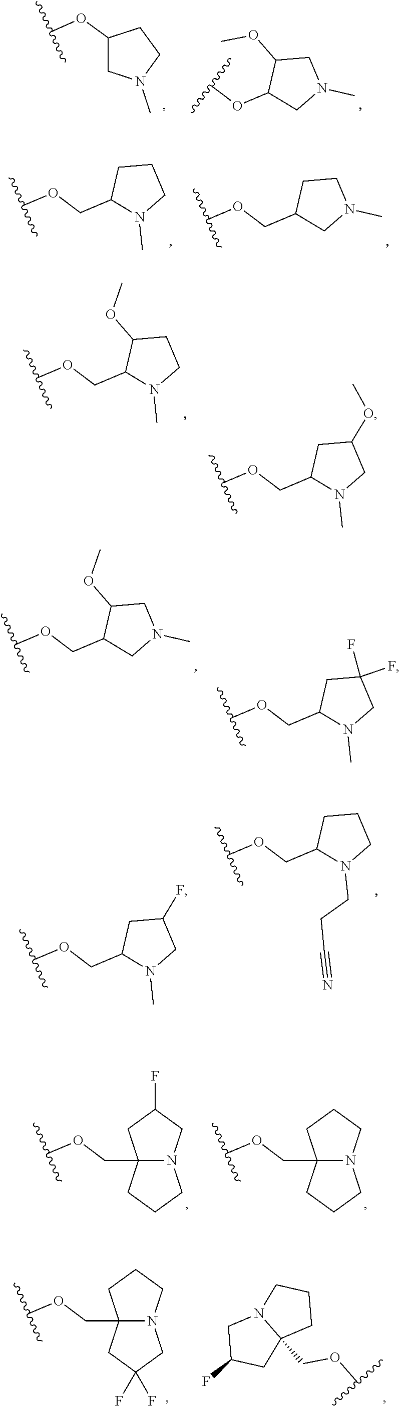

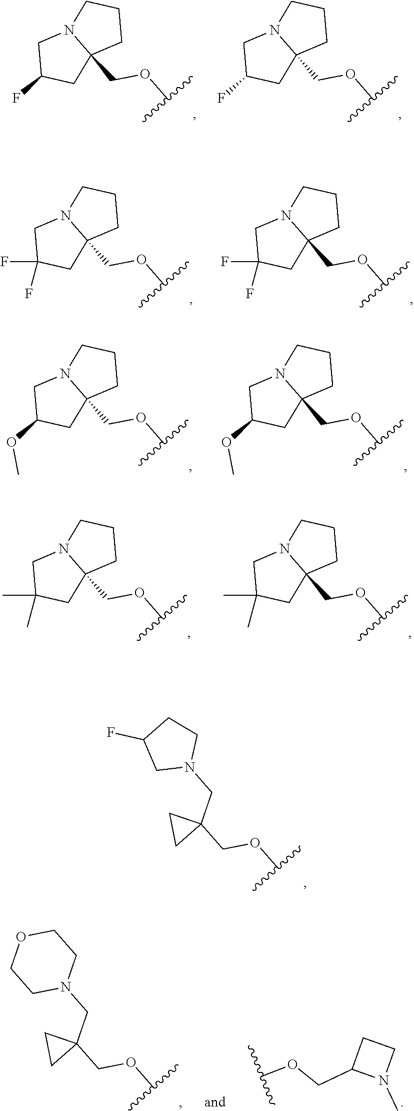

In some embodiments, —OR⁸ is selected from:

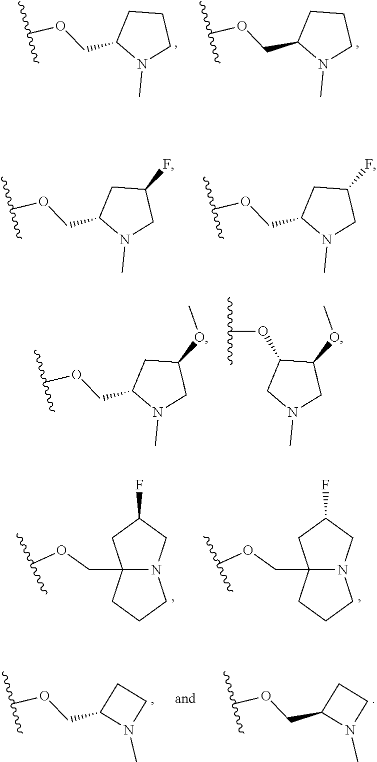

In some embodiments, R⁶ is selected from:

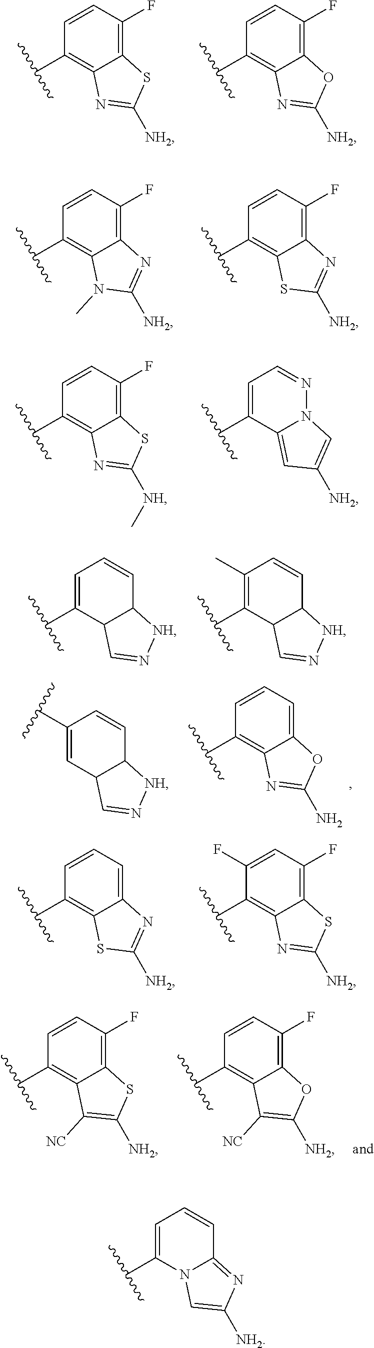

In some embodiments, R⁶ is selected from:

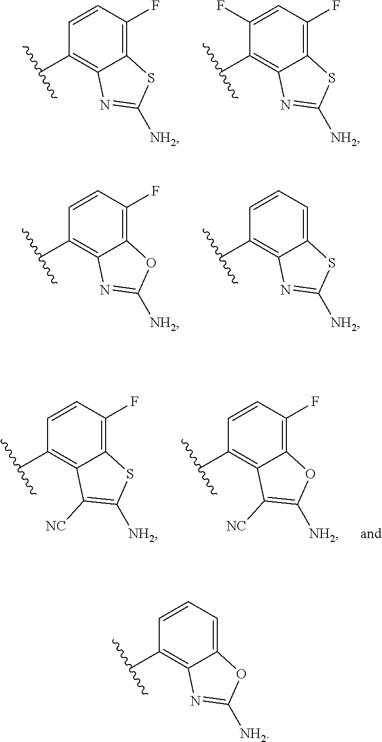

In some embodiments, R⁶ is:

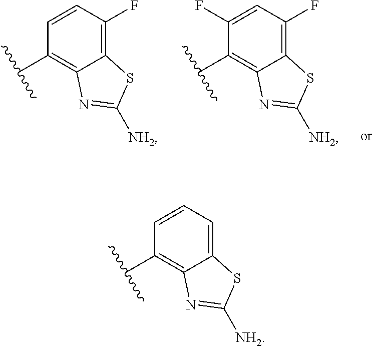 [SMILES: *c1ccc(F)c2sc(N)nc12 ; *c1c(F)cc(F)c2sc(N)nc12 ; "or" ; *c1cccc2sc(N)nc12]

In some embodiments, R⁴ is H. In some embodiments, R⁴ is a halogen.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, (i) Ring A is a piperazine or diazepane that is unsubstituted or is substituted with one or more R¹¹. In some embodiments, (ii) R⁷ is a halogen. In some embodiments, (iii) R⁴ is H. In some embodiments, Ring A is selected from:

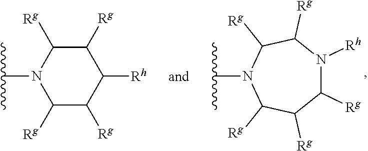

wherein each R⁹ is independently selected from H and C₁₋₆alkyl and R^{h} is selected from H, C₁₋₆alkyl, —C(O)NH₂, and —C(O)C₁₋₆alkylNH₂. In some embodiments, at least one R⁹ is C₁₋₆alkyl, such as C₁alkyl. In some embodiments, R^{h} is H. In some embodiments, Ring A is selected from:

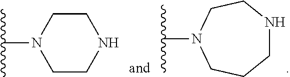 [SMILES: *N1CCNCC1 ; *N1CCCNCC1]

In some embodiments, R⁸ is an alkylheterocycle. In some embodiments, —OR⁸ is selected from:

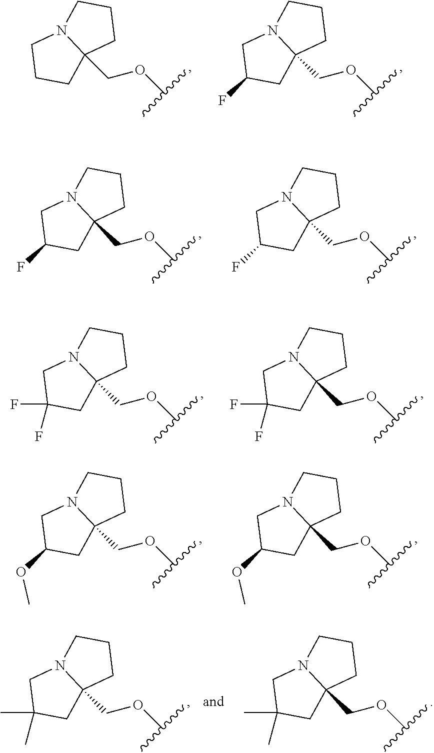

In some embodiments, —OR⁸ is selected from:

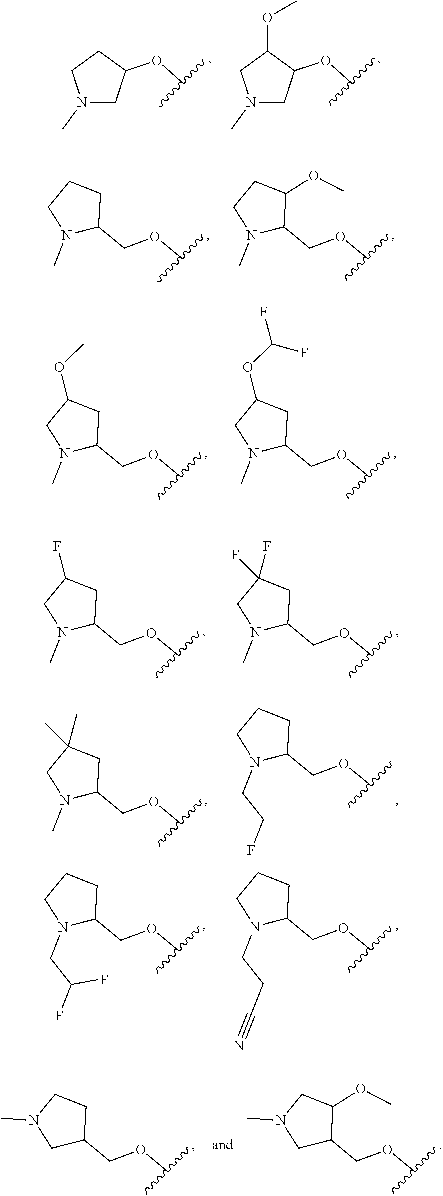

In some embodiments, R⁷ is F. In some embodiments, R⁵ is selected from a halogen, —CN, C₁₋₆alkyl, and 3-6 membered carbocycle, which carbocycle or C₁₋₆alkyl is unsubstituted or is substituted with one or more R¹³. In some embodiments, R⁶ is.

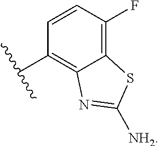 [SMILES: *c1ccc(F)c2sc(N)nc12]

In some embodiments, (i) R⁸ is an alkylheterocycle; and (ii) Ring A is a piperazine that is unsubstituted or is substituted with one or more R¹¹. In some embodiments, (i) R⁸ is an alkylheterocycle; and (ii) Ring A is a diazepane that is unsubstituted or is substituted with one or more R¹¹. In some embodiments, (i) R⁸ is an alkylheterocycle; and (ii) Ring A is a piperazine or diazepane that is unsubstituted.

In some embodiments, (i) Ring A is a bridged heterocyclic ring system that is unsubstituted or is substituted with one or more R¹¹. In some embodiments, (ii) R⁷ is a halogen. In some embodiments, (iii) R⁴ is H. In some embodiments, Ring A is selected from:

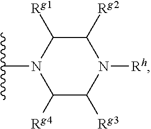

wherein (a) R^{g1} and R^{g3} or (b) R^{g2} and R^{g3} join together to form a second ring containing 4-6 members, and wherein any of R^{g1}, R^{g2}, R^{g3}, and R^{g4} that are not part of the second ring are independently selected from H and C₁₋₆alkyl; and R^{h} is selected from H, C₁₋₆alkyl, —C(O)NH₂, and —C(O)C₁₋₆alkylNH₂. In some embodiments, at least one of R^{g1}, R^{g2}, R^{g3}, and R^{g4} is C₁₋₆alkyl, such as C₁alkyl. In some embodiments, R^{h} is H. In some embodiments, Ring A is selected from:

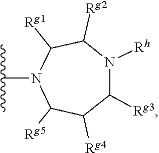

wherein R^{g2} and R^{g4} join together to form a second ring containing 4-6 members; R^{g1}, R^{g3}, and R^{g5} are independently selected from H and C₁₋₆alkyl; and R^{h} is selected from H, C₁₋₆alkyl, —C(O)NH₂, and —C(O)C₁₋₆alkylNH₂. In some embodiments, at least one of R^{g1}, R^{g3}, and R^{g5} is C₁₋₆alkyl, such as C₁alkyl. In some embodiments, R^{h} is H. In some embodiments, R^{h} is —C(O) NH₂. In some embodiments, Ring A is selected from:

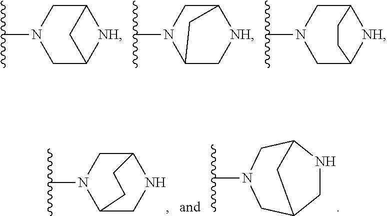

In some embodiments, R⁸ is an alkylheterocycle. In some embodiments, —OR⁸ is selected from:

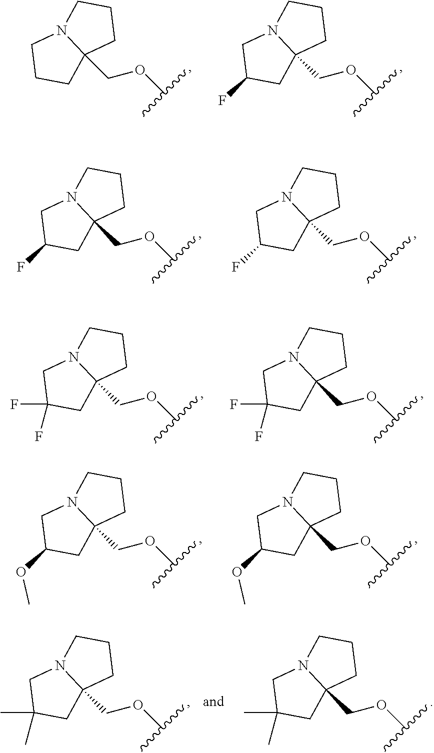

In some embodiments, —OR⁸ is selected from:

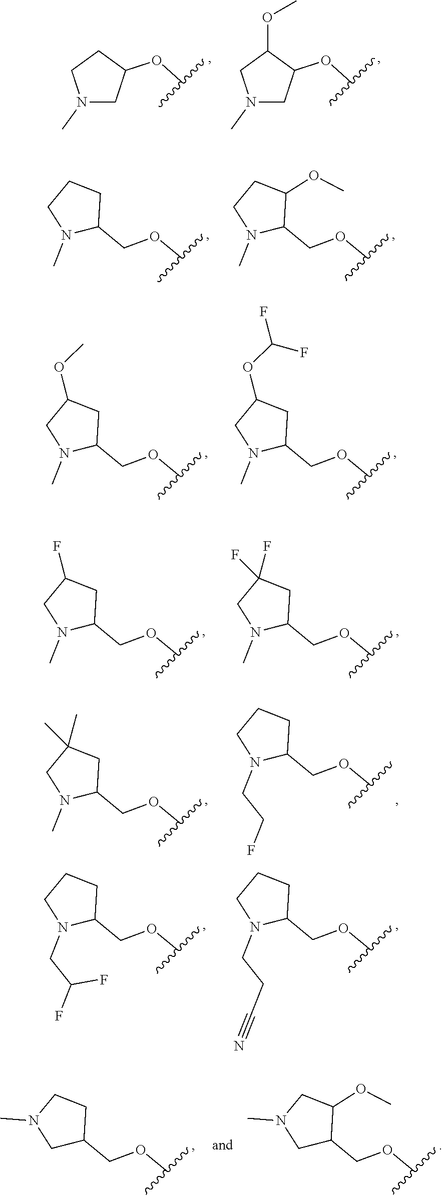

In some embodiments, R⁷ is F. In some embodiments, R⁵ is selected from a halogen, —CN, C₁₋₆alkyl, and 3-6 membered carbocycle, which carbocycle or C₁₋₆alkyl is unsubstituted or is substituted with one or more R¹³. In some embodiments, R⁶ is.

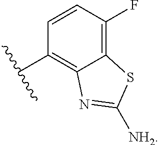 [SMILES: *c1ccc(F)c2sc(N)nc12]

In some embodiments, (i) R⁸ is an alkylheterocycle; (ii) Ring A is a bridged heterocyclic ring system that is unsubstituted or is substituted with one or more R¹¹; (iii) R⁷ is a halogen; and (iv) R⁴ is H.

In another aspect, the present disclosure provides a compound according to Formula IC:

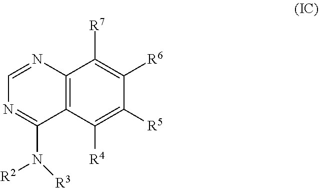

or a salt (e.g., pharmaceutically acceptable salt), ester, tautomer, prodrug, zwitterionic form, or stereoisomer thereof, wherein R², R³, R⁴, R⁵, R⁶, and R⁷ are as defined above for Formula I and described in classes and subclasses herein, both singly and in combination. In some embodiments, the present disclosure provides a compound of Formula IC, or a salt (e.g., a pharmaceutically acceptable salt) thereof.

In some embodiments, the present disclosure provides a compound of Formula IC, wherein:
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, R² is H. In some embodiments, R² is C₁₋₆ alkyl. In some embodiments, R² is a 3-6 membered carbocycle. In some embodiments, R² is cyclopropyl.

In some embodiments, R³ is a carbocycle, which carbocycle is unsubstituted or substituted with one or more R¹⁰. In some embodiments, R³ is a multicyclic carbocycle. In some embodiments, R³ is a monocyclic carbocycle. In some embodiments, R³ is a carbocycle that is substituted with —N(R¹⁹)₂, wherein each R¹⁹ is independently selected from unsubstituted or substituted C₁₋₆ alkyl and H. In some embodiments, R³ is a carbocycle that is substituted with —NH₂.

In some embodiments, R³ is a heterocycle, which heterocycle is unsubstituted or substituted with one or more R¹⁰. In some embodiments, R³ is a multicyclic heterocycle. In some embodiments, R³ is a monocyclic heterocycle. In some embodiments, the heterocycle includes one or more nitrogen atoms. In some embodiments, R³ is a heterocycle that is substituted with —N(R¹⁹)₂, wherein each R¹⁹ is independently selected from unsubstituted or substituted C₁₋₆ alkyl and H. In some embodiments, R³ is a heterocycle that is substituted with —NH₂.

In some embodiments, R³ is selected from:

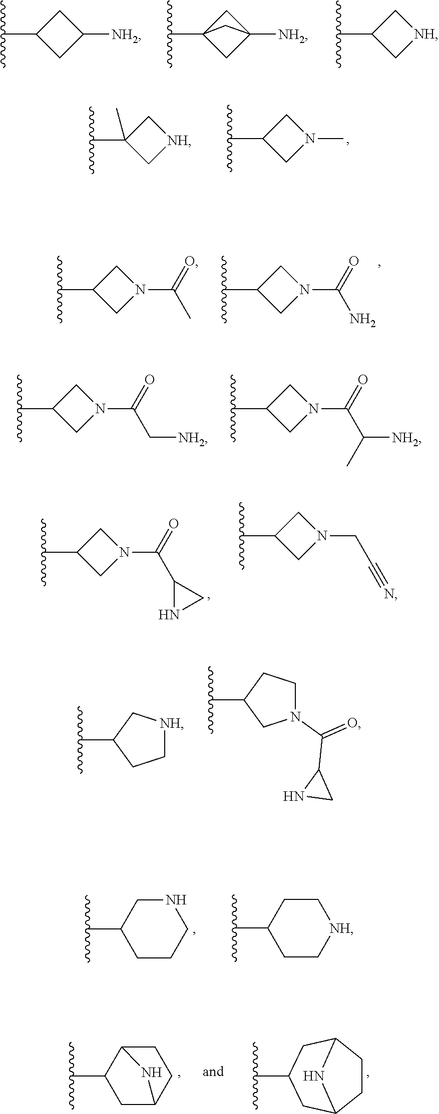

any of which is unsubstituted or substituted with one or more R¹⁰.

In some embodiments, R³ is selected from:

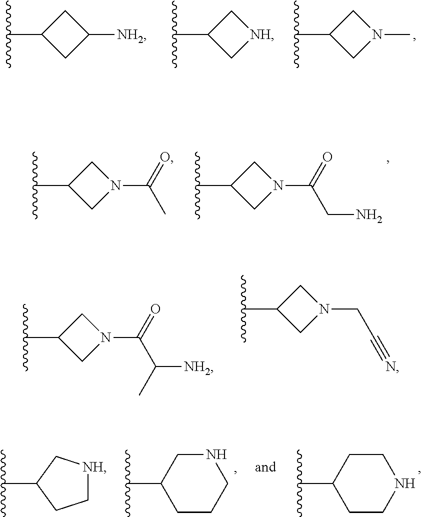

any of which is unsubstituted or substituted with one or more R¹⁰.

In some embodiments, R³ is selected from:

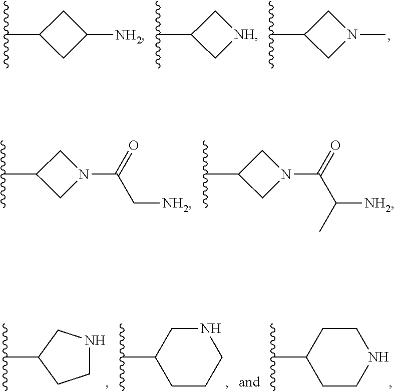

any of which is unsubstituted or substituted with one or more R¹⁰.

In some embodiments, R³ is selected from C₁₋₆ alkyl that is substituted with one or more R⁹. In some embodiments, R³ is selected from C₁₋₆alkyl-N(R¹⁷)₂, wherein each R¹⁷ is independently selected from C₁₋₆ alkyl and H. In some embodiments, R³ is C₁₋₆ alkyl substituted with —NH₂.

In some embodiments, R³ is selected from:

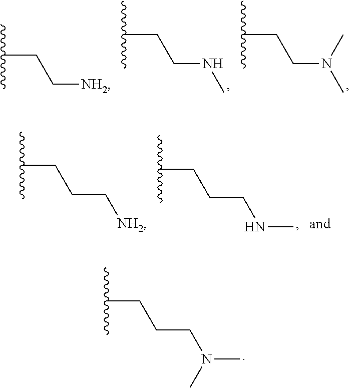

In some embodiments, R³ includes an amino moiety (e.g., —NRR′). In some embodiments, the amino moiety is a component of a heterocycle. In some embodiments, the amino moiety is appended to a carbocycle or heterocycle. In some embodiments, the amino moiety is a primary amine (e.g., —NH₂). In some embodiments, the amino moiety is a secondary amine (e.g., —NHR).

In some embodiments, R³ is selected from C₁₋₆ alkyl that is substituted with one or more R⁹. In some embodiments, R³ is C₁₋₆ alkyl substituted with —N(R¹⁷)₂, wherein each R¹⁷ is independently selected from C₁₋₆ alkyl and H. In some embodiments, R³ is C₁₋₆ alkyl substituted with —NH₂. In some embodiments, R³ is selected from:

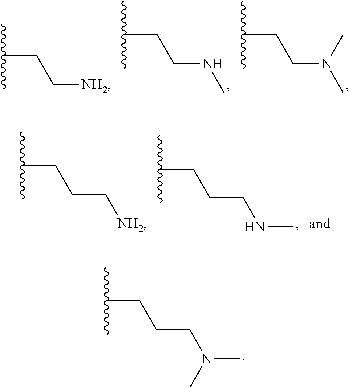

In some embodiments, R² or R³ includes an amino moiety (e.g., —NRR′).

In some embodiments, R² and R³, together with the atom to which they are attached, form a monocyclic heterocycle that is unsubstituted or substituted with one or more R¹¹. In some embodiments, R² and R³, together with the atom to which they are attached, form a multicyclic heterocycle that is unsubstituted or substituted with one or more R¹¹. In some embodiments, R² and R³, together with the atom to which they are attached, form a 4-10 membered heterocycle that is unsubstituted or substituted with one or more R¹¹. In some embodiments, R² and R³, together with the atom to which they are attached, form a 4-6 membered heterocycle that is unsubstituted or substituted with one or more R¹¹. In some embodiments, R² and R³, together with the atom to which they are attached, form a structure that includes at least one nitrogen atom. In some embodiments, R² and R³, together with the atom to which they are attached, form a ring having the structure:

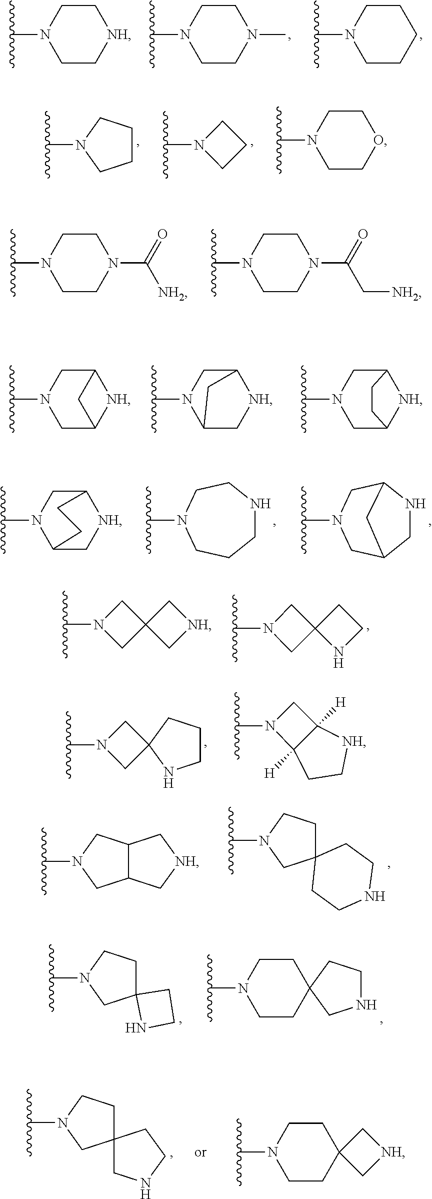

any of which is unsubstituted or substituted with one or more R¹¹. In some embodiments, R² and R³, together with the atom to which they are attached, form a ring having the structure:

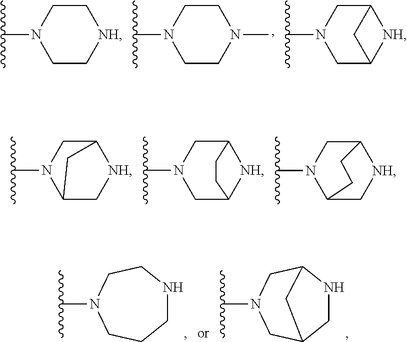

which ring is unsubstituted or substituted with one or more R¹¹. In some embodiments, R² and R³, together with the atom to which they are attached, form a ring having the structure:

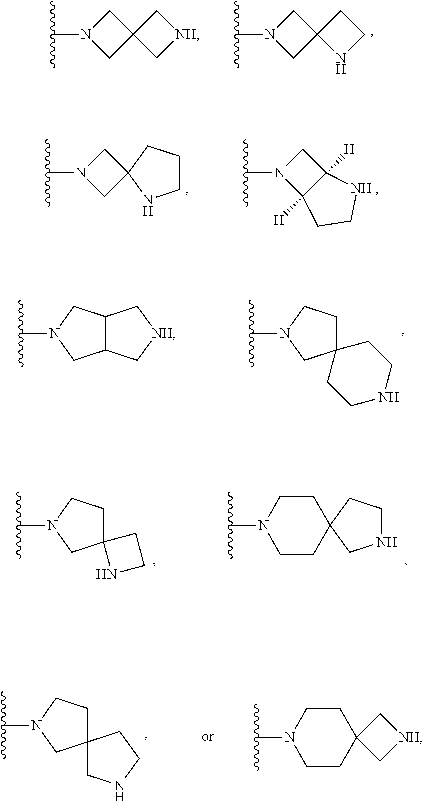

which ring is unsubstituted or substituted with one or more R¹¹. In some embodiments, R² and R³, together with the atom to which they are attached, form a ring having the structure:

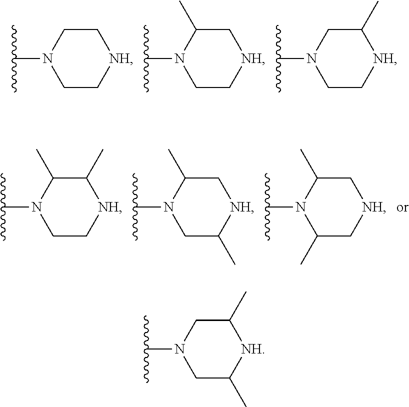

In some embodiments, R² and R³, together with the atom to which they are attached, form a ring having the structure:

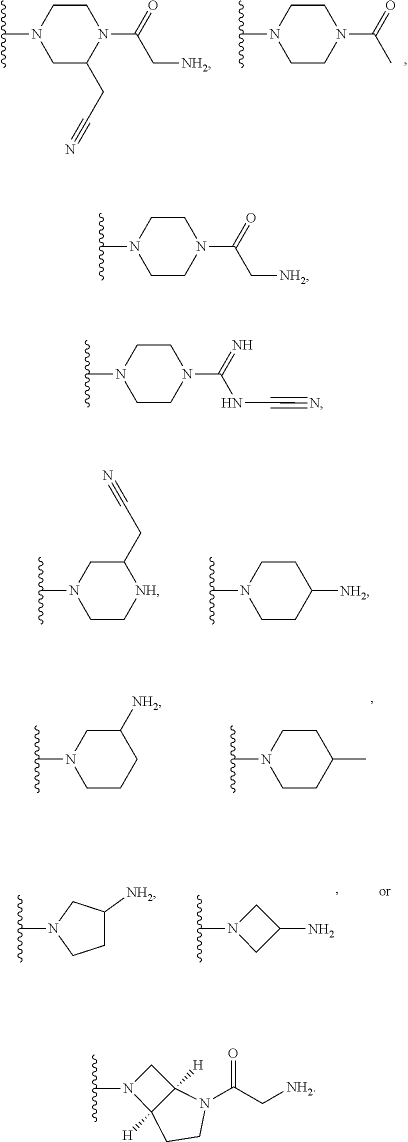

In some embodiments, R² and R³, together with the atom to which they are attached, form a 4-10 membered heterocycle that includes at least two nitrogen atoms (e.g., includes an amino moiety (e.g., —NR—)) and/or is substituted with a group including an amino moiety (e.g., —NRR′). In some embodiments, R² and R³, together with the atom to which they are attached, form a 4-6 membered heterocycle that includes at least two nitrogen atoms (e.g., includes an amino moiety (e.g., —NR—)) and/or is substituted with a group including an amino moiety (e.g., —NRR′).

In some embodiments, R⁶ is selected from:

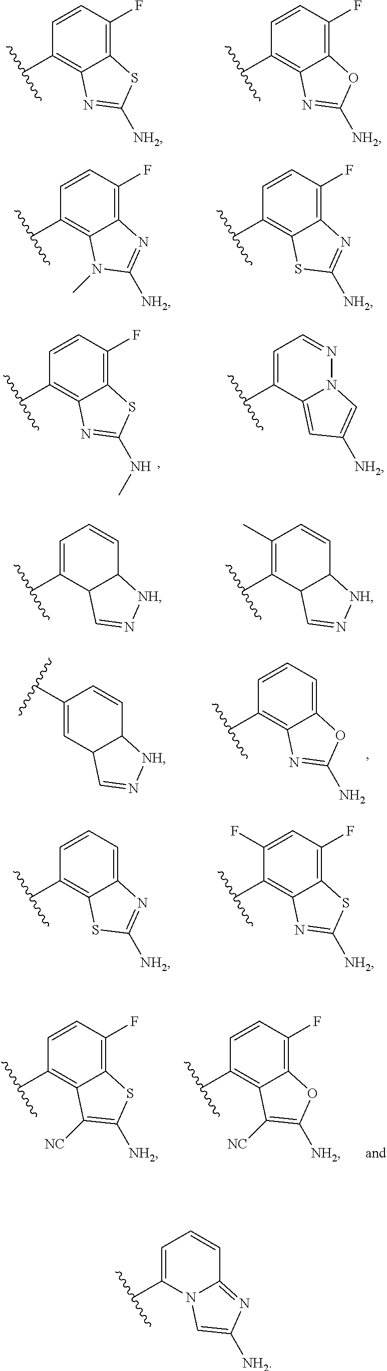

In some embodiments, R⁶ is selected from:

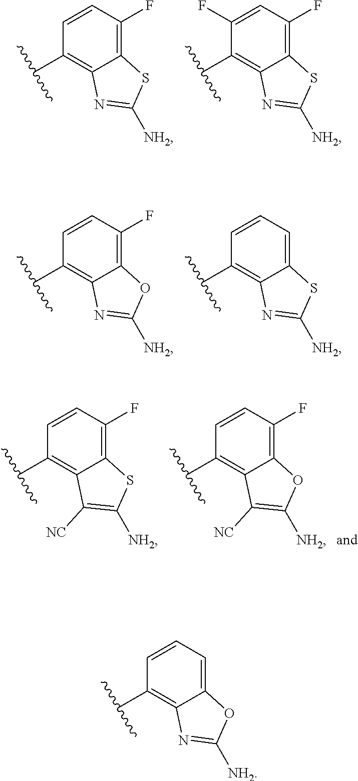

In some embodiments, R⁶ is:

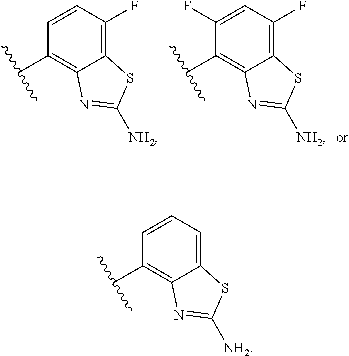 [SMILES: *c1ccc(F)c2sc(N)nc12 ; *c1c(F)cc(F)c2sc(N)nc12 ; *c1cccc2sc(N)nc12]

In some embodiments, R⁴ is H. In some embodiments, R⁴ is a halogen.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In another aspect, the present disclosure provides a compound according to Formula ID or ID′:

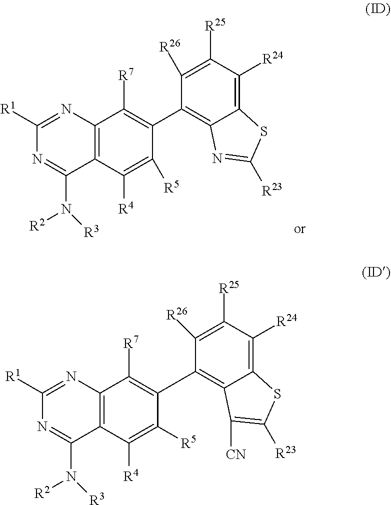

or a salt (e.g., pharmaceutically acceptable salt), ester, tautomer, prodrug, zwitterionic form, or stereoisomer thereof, wherein R¹, R², R³, R⁴, R⁵, and R⁷ are as defined above for Formula I and described in classes and subclasses herein, both singly and in combination; R²³ is selected from —N(R¹²)₂ and C₁₋₆alkyl-N(R¹²)₂; and R²⁴, R²⁵, and R²⁶ are independently selected from H, halogen, —OR¹², and C₁₋₆alkyl, wherein any C₁₋₆alkyl is unsubstituted or substituted with one or more R¹¹. In some embodiments, the present disclosure provides a compound of Formula ID or a salt (e.g., pharmaceutically acceptable salt) thereof. In some embodiments, the present disclosure provides a compound of Formula ID′ or a salt (e.g., pharmaceutically acceptable salt) thereof.

In some embodiments, the present disclosure provides a compound of Formula ID or ID′, wherein:
R¹ is selected from —OR⁸, a 4-6 membered heterocycle including a nitrogen atom, and H, wherein the heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰; or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.
R²³ is selected from —N(R¹²)₂ and C₁₋₆alkyl-N(R¹²)₂; and
R²⁴, R²⁵, and R²⁶ are independently selected from H, halogen, —OR¹², and C₁₋₆alkyl, wherein any C₁₋₆alkyl is unsubstituted or substituted with one or more R¹¹.

In some embodiments, R² is H. In some embodiments, R² is C₁₋₆ alkyl. In some embodiments, R² is a 3-6 membered carbocycle. In some embodiments, R² is cyclopropyl.

In some embodiments, R³ is a carbocycle, which carbocycle is unsubstituted or substituted with one or more R¹⁰. In some embodiments, R³ is a multicyclic carbocycle. In some embodiments, R³ is a monocyclic carbocycle. In some embodiments, R³ is a carbocycle that is substituted with —N(R¹⁹)₂, wherein each R¹⁹ is independently selected from unsubstituted or substituted C₁₋₆ alkyl and H. In some embodiments, R³ is a carbocycle that is substituted with —NH₂.

In some embodiments, R³ is a heterocycle, which heterocycle is unsubstituted or substituted with one or more R¹⁰. In some embodiments, R³ is a multicyclic heterocycle. In some embodiments, R³ is a monocyclic heterocycle. In some embodiments, the heterocycle includes one or more nitrogen atoms. In some embodiments, R³ is a heterocycle that is substituted with —N(R¹⁹)₂, wherein each R¹⁹ is independently selected from unsubstituted or substituted C₁₋₆ alkyl and H. In some embodiments, R³ is a heterocycle that is substituted with —NH₂.

In some embodiments, R³ is selected from:

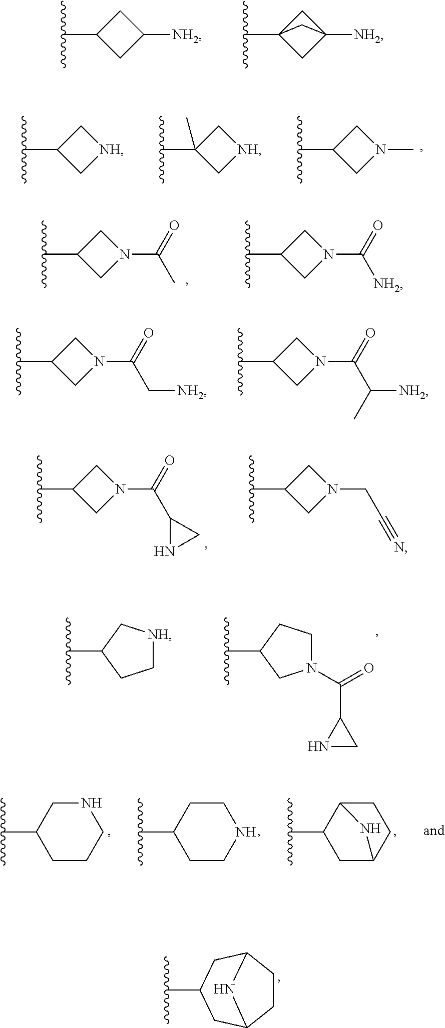

any of which is unsubstituted or substituted with one or more R¹⁰.

In some embodiments, R³ is selected from:

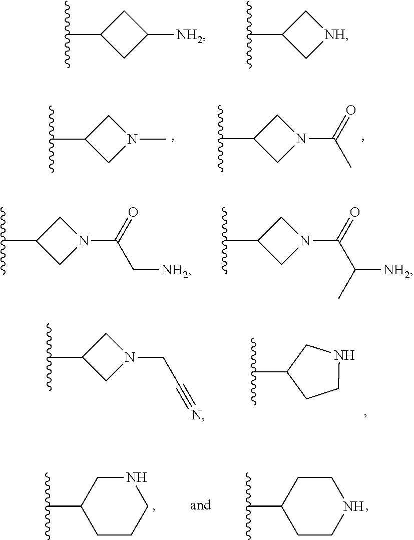

any of which is unsubstituted or substituted with one or more R¹⁰.

In some embodiments, R³ is selected from:

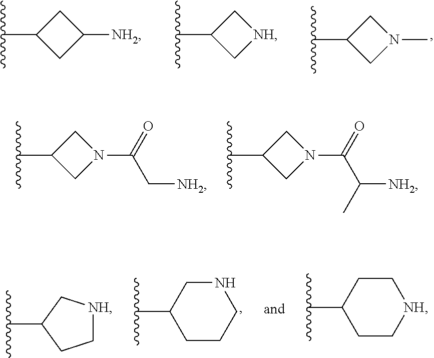

any of which is unsubstituted or substituted with one or more R¹⁰.

In some embodiments, R³ is selected from C₁₋₆ alkyl that is substituted with one or more R⁹. In some embodiments, R³ is selected from C₁₋₆alkyl-N(R¹⁷)₂, wherein each R⁷ is independently selected from C₁₋₆ alkyl and H. In some embodiments, R³ is C₁₋₆ alkyl substituted with —NH₂.

In some embodiments, R³ is selected from:

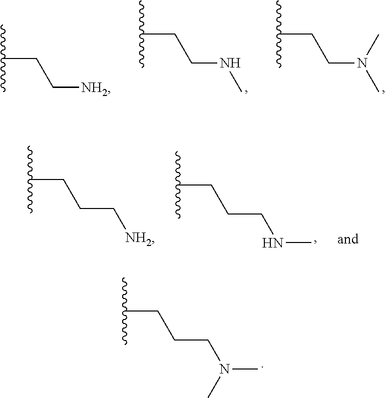

In some embodiments, R³ includes an amino moiety (e.g., —NRR′). In some embodiments, the amino moiety is a component of a heterocycle. In some embodiments, the amino moiety is appended to a carbocycle or heterocycle. In some embodiments, the amino moiety is a primary amine (e.g., —NH₂). In some embodiments, the amino moiety is a secondary amine (e.g., —NHR).

In some embodiments, R³ is selected from C₁₋₆ alkyl that is substituted with one or more R⁹. In some embodiments, R³ is C₁₋₆ alkyl substituted with —N(R¹⁷)₂, wherein each R⁷ is independently selected from C₁₋₆ alkyl and H. In some embodiments, R³ is C₁₋₆ alkyl substituted with —NH₂. In some embodiments, R³ is selected from:

In some embodiments, R² or R³ includes an amino moiety (e.g., —NRR′).

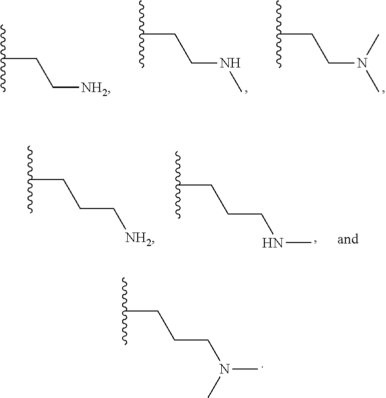

In some embodiments, R² and R³, together with the atom to which they are attached, form a monocyclic heterocycle that is unsubstituted or substituted with one or more R¹¹. In some embodiments, R² and R³, together with the atom to which they are attached, form a multicyclic heterocycle that is unsubstituted or substituted with one or more R¹¹. In some embodiments, R² and R³, together with the atom to which they are attached, form a 4-10 membered heterocycle that is unsubstituted or substituted with one or more R¹¹. In some embodiments, R² and R³, together with the atom to which they are attached, form a 4-6 membered heterocycle that is unsubstituted or substituted with one or more R¹¹. In some embodiments, R² and R³, together with the atom to which they are attached, form a structure that includes at least one nitrogen atom. In some embodiments, R² and R³, together with the atom to which they are attached, form a ring having the structure:

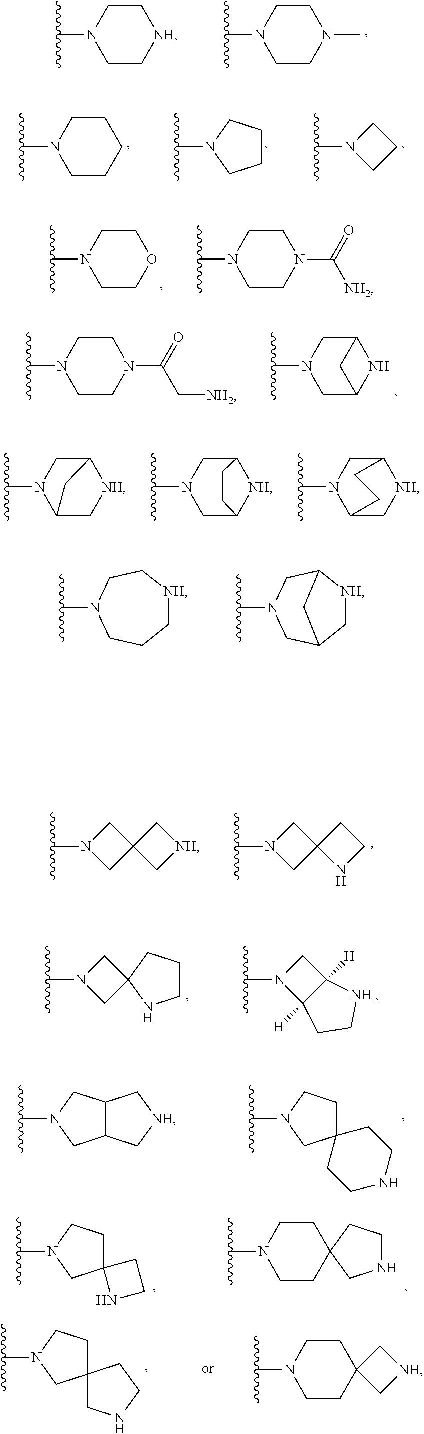

any of which is unsubstituted or substituted with one or more R¹¹. In some embodiments, R² and R³, together with the atom to which they are attached, form a ring having the structure:

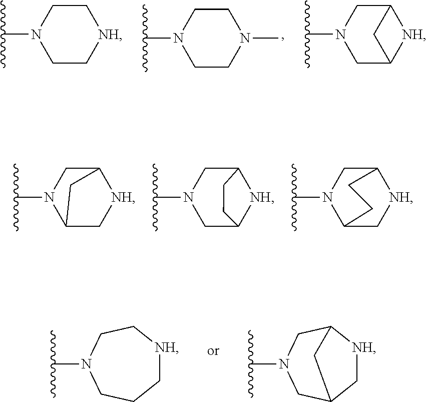

which ring is unsubstituted or substituted with one or more R¹¹. In some embodiments, R² and R³, together with the atom to which they are attached, form a ring having the structure:

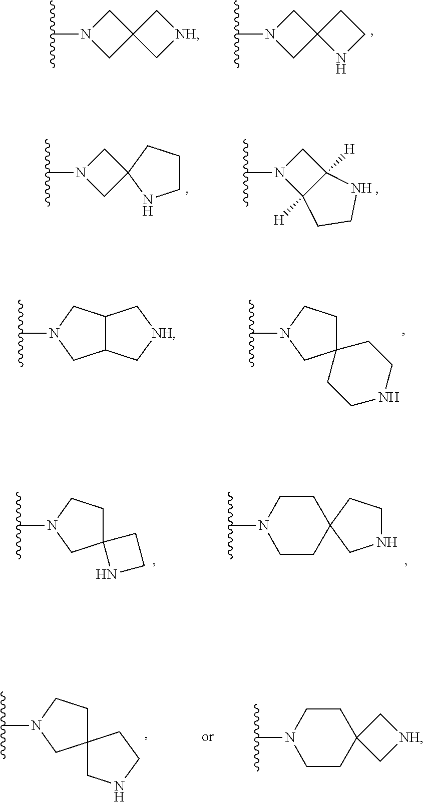

which ring is unsubstituted or substituted with one or more R¹¹. In some embodiments, R² and R³, together with the atom to which they are attached, form a ring having the structure:

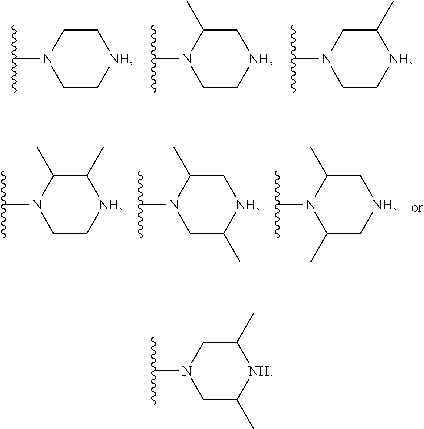

In some embodiments, R² and R³, together with the atom to which they are attached, form a ring having the structure:

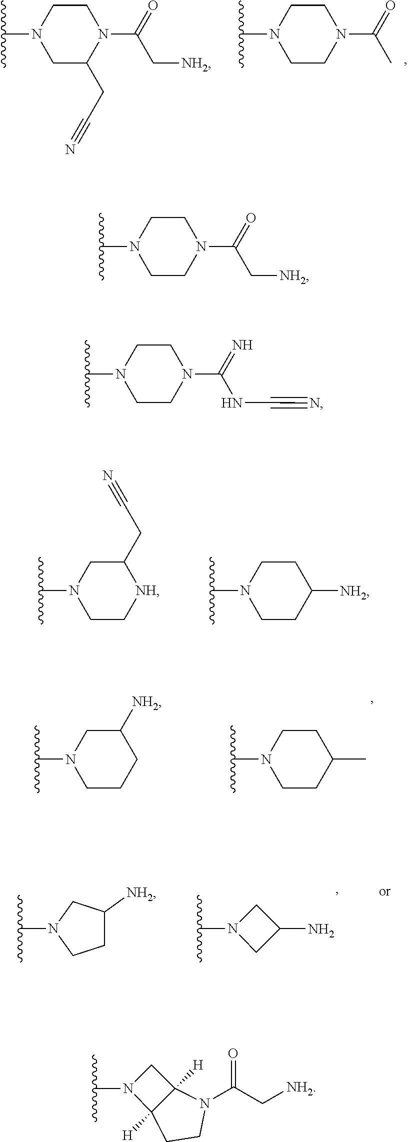

In some embodiments, R² and R³, together with the atom to which they are attached, form a 4-10 membered heterocycle that includes at least two nitrogen atoms (e.g., includes an amino moiety (e.g., —NR—)) and/or is substituted with a group including an amino moiety (e.g., —NRR′). In some embodiments, R² and R³, together with the atom to which they are attached, form a 4-6 membered heterocycle that includes at least two nitrogen atoms (e.g., includes an amino moiety (e.g., —NR—)) and/or is substituted with a group including an amino moiety (e.g., —NRR′).

In some embodiments, R¹ is H.

In some embodiments, R¹ is —OR⁸. In some embodiments, R⁸ is a heterocycle. In some embodiments, R⁸ is an alkylheterocycle. In some embodiments, a heterocycle or a heterocycle of an alkylheterocycle comprises 4-8 members including at least one heteroatom selected from N, O, and S. In some embodiments, R⁸ comprises a heterocycle including at least one nitrogen atom. In some embodiments, R⁸ comprises a 4-8 membered heterocycle including at least one nitrogen atom. In some embodiments, R⁸ comprises a 3-6 membered heterocycle that is unsubstituted or substituted with one or more R¹⁶. In some embodiments, the heterocycle comprises one or more R¹⁶ substituents. In some embodiments, at least one R¹⁶ is —OR¹², wherein R¹² is independently selected from C₁₋₆ alkyl, C₂₋₆ alkenyl, and H. In some embodiments, at least one R¹⁶ is —OCH₃.

In some embodiments, R¹ is selected from:

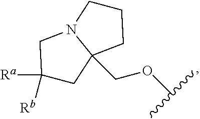

wherein R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R¹ is selected from:

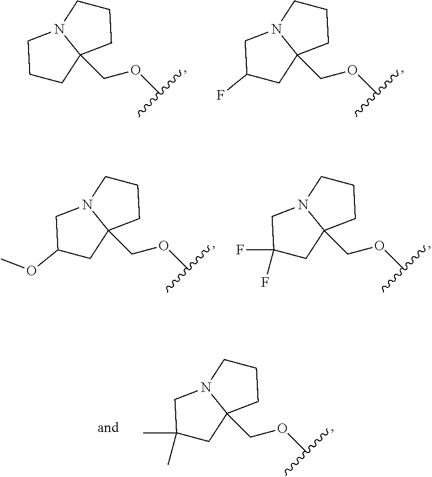 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, R¹ is selected from:

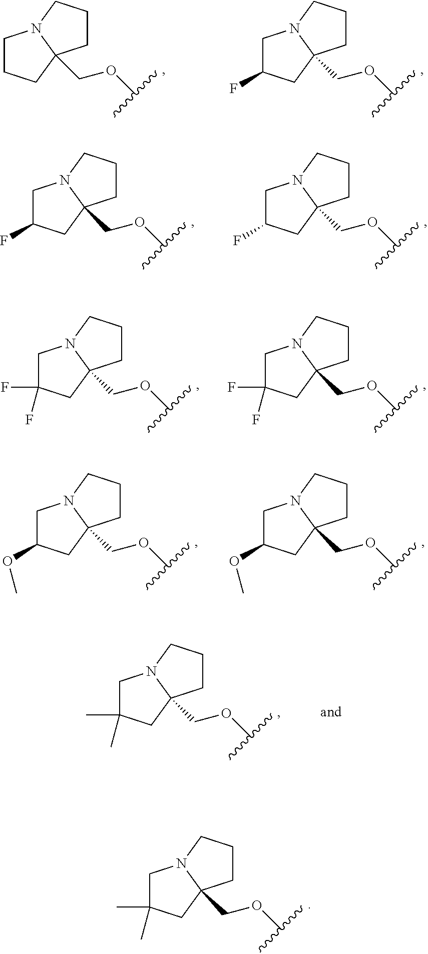

In some embodiments, R¹ is selected from:

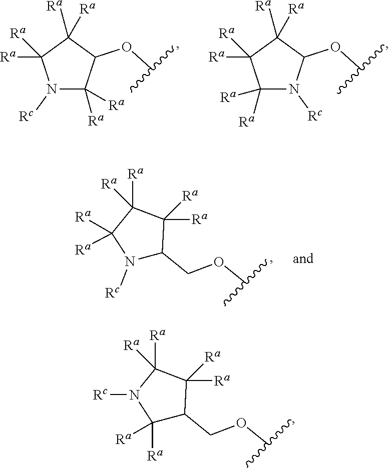

wherein each R^{a} is independently selected from halogen, C₁₋₆ alkyl, —OR¹², and H; and wherein R^{c} is selected from C₁₋₆ alkyl, wherein a C₁₋₆ alkyl of a R^{a} or R^{c} is unsubstituted or is substituted with one or more R¹³. In some embodiments, R¹ is selected from:

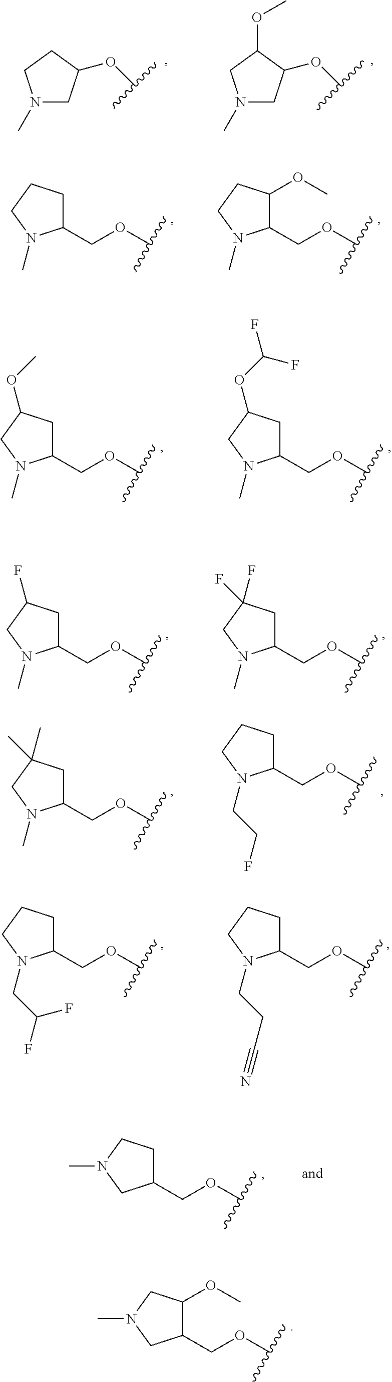

In some embodiments, R¹ is selected from:

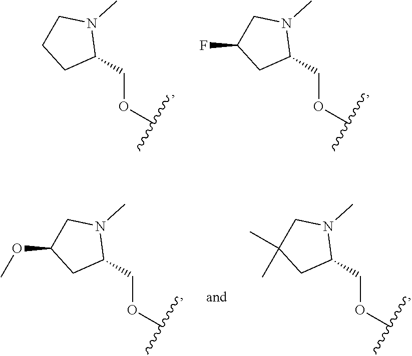

In some embodiments, R¹ is selected from:

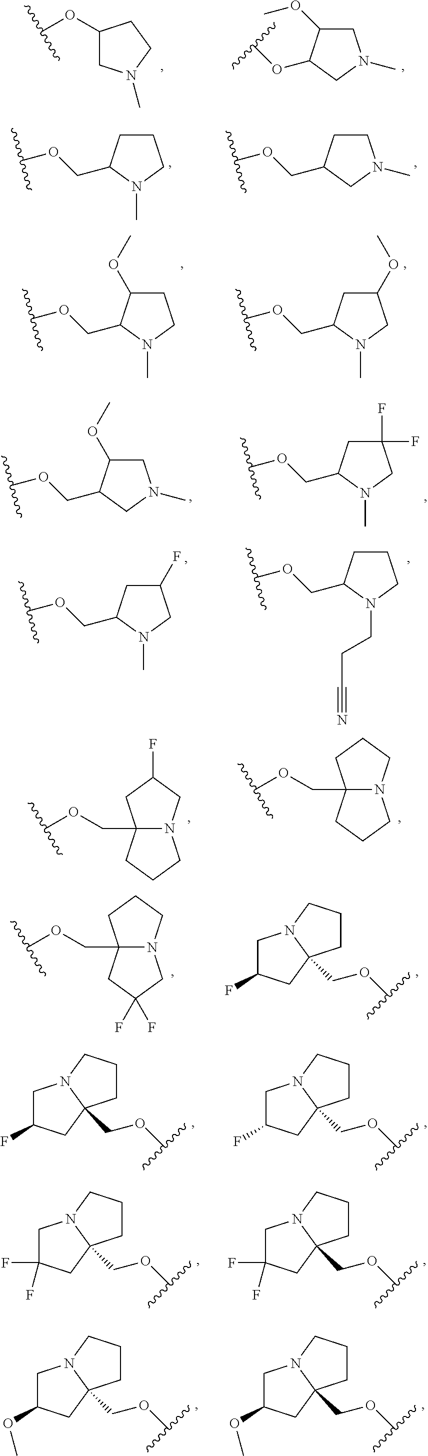

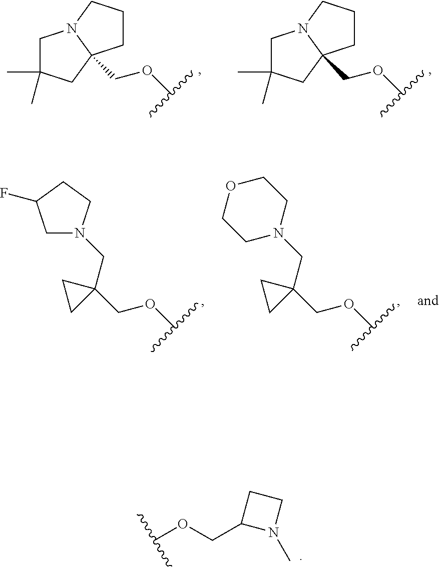

In some embodiments, R¹ is selected from:

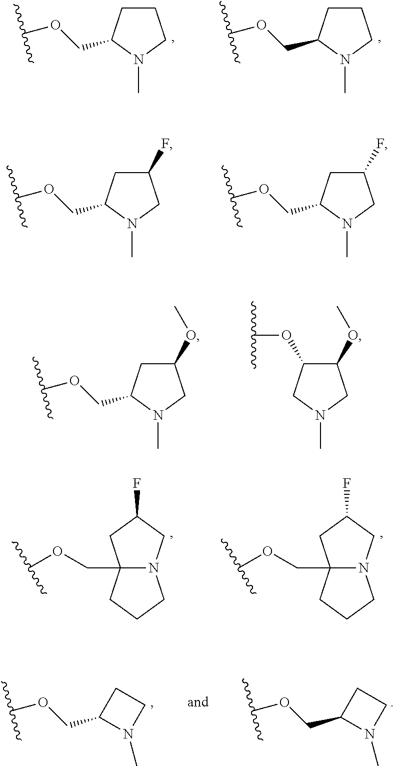

In some embodiments, R¹ is a 4-6 membered heterocycle including a nitrogen atom that is unsubstituted or substituted with one or more R¹⁶. In some embodiments, R¹ is selected from:

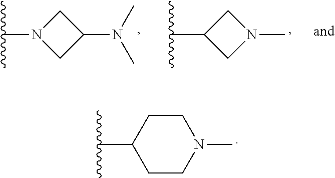

In some embodiments, R⁴ is H. In some embodiments, R⁴ is a halogen.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, R²³ is —N(R¹²)₂. In some embodiments, R²³ is —NH₂.

In some embodiments, R²⁴ is a halogen. In some embodiments, R²⁴ is Cl or F. In some embodiments, R²⁴ is F. In some embodiments, R²⁴ is H.

In some embodiments, R²⁵ and R²⁶ are H. In some embodiments, R²⁵ is H. In some embodiments, R²⁶ is H.

In an aspect, the present disclosure provides a compound represented by Formula IE:

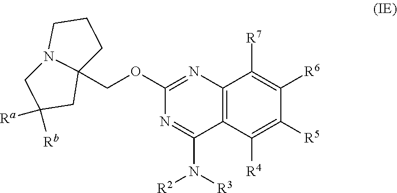

or a salt (e.g., pharmaceutically acceptable salt), ester, tautomer, prodrug, zwitterionic form, or stereoisomer thereof, wherein R², R³, R⁴, R⁵, R⁶, and R⁷ are as defined above for Formula I and described in classes and subclasses herein, both singly and in combination; and R^{a} and R^{b} are independently selected from halogen, —OR¹², C₁₋₆alkyl, and H, wherein any C₁₋₆alkyl is unsubstituted or is substituted with one or more R¹³. In some embodiments, the present disclosure provides a compound of Formula IE, or a salt (e.g., a pharmaceutically acceptable salt) thereof.

In some embodiments, the present disclosure provides a compound of Formula IE, wherein:
R² is selected from H, a 3-6 membered carbocycle, and C₁₋₆ alkyl, wherein the 3-6 membered carbocycle or the C₁₋₆ alkyl is unsubstituted or substituted with one or more R¹³;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen;
each R²² is independently selected from C₁₋₆ alkyl, C₂₋₆ alkenyl, and H; and
R^{a} and R^{b} are independently selected from halogen, —OR¹², C₁₋₆alkyl, and H, wherein any C₁₋₆alkyl is unsubstituted or is substituted with one or more R¹³.

In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{a} and R^{b} are both halogens. In some embodiments, R^{a} and R^{b} are both F. In some embodiments, R^{a} and R^{b} are both C₁₋₆alkyl that is unsubstituted or is substituted with one or more R¹³. In some embodiments, R^{a} and R^{b} are both methyl. In some embodiments, R^{a} and R^{b} are both H. In some embodiments, R^{a} is —OC₁₋₆alkyl and R^{b} is H.

In some embodiments, R² is H. In some embodiments, R² is C₁₋₆ alkyl. In some embodiments, R² is a 3-6 membered carbocycle. In some embodiments, R² is cyclopropyl.

In some embodiments, R³ is C₁₋₆ alkyl, which C₁₋₆alkyl is substituted with one or more R⁹. In some embodiments, R³ is C₁₋₆ alkyl substituted with —N(R⁷)₂, wherein each R⁷ is independently selected from C₁₋₆ alkyl and H. In some embodiments, R³ is C₁₋₆ alkyl substituted with —NH₂.

In some embodiments, R³ is selected from:

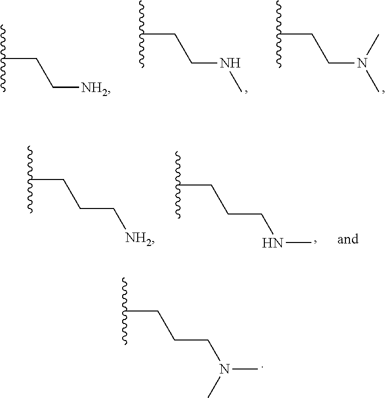

In some embodiments, R³ includes an amino moiety (e.g., —NRR′). In some embodiments, the amino moiety is a component of a heterocycle. In some embodiments, the amino moiety is appended to a carbocycle or heterocycle. In some embodiments, the amino moiety is a primary amine (e.g., —NH₂). In some embodiments, the amino moiety is a secondary amine (e.g., —NHR).

In some embodiments, R³ is a carbocycle, which carbocycle is unsubstituted or substituted with one or more R¹⁰. In some embodiments, R³ is a multicyclic carbocycle. In some embodiments, R³ is a monocyclic carbocycle. In some embodiments, R³ is a carbocycle that is substituted with —N(R¹⁹)₂, wherein each R¹⁹ is independently selected from unsubstituted or substituted C₁₋₆ alkyl and H. In some embodiments, R³ is a carbocycle that is substituted with —NH₂.

In some embodiments, R³ is a heterocycle, which heterocycle is unsubstituted or substituted with one or more R¹⁰. In some embodiments, R³ is a multicyclic heterocycle. In some embodiments, R³ is a monocyclic heterocycle. In some embodiments, the heterocycle includes one or more nitrogen atoms. In some embodiments, R³ is a heterocycle that is substituted with —N(R¹⁹)₂, wherein each R¹⁹ is independently selected from unsubstituted or substituted C₁₋₆ alkyl and H. In some embodiments, R³ is a heterocycle that is substituted with —NH₂. In some embodiments, R³ is a bridged heterocyclic ring system. In some embodiments, R³ is a monocycle. In some embodiments, R³ is an azetidine or a pyrrolidine that is unsubstituted or substituted with one or more R¹⁰.

In some embodiments, R³ is selected from:

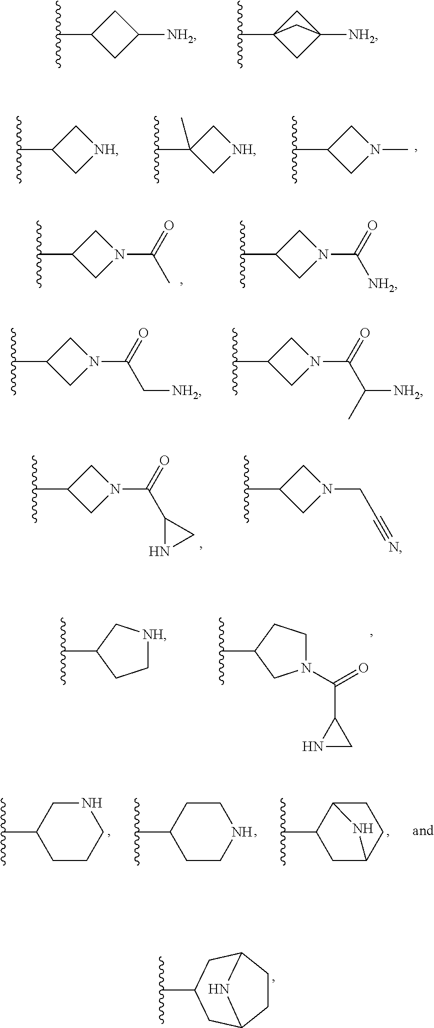

any of which is unsubstituted or substituted with one or more R¹⁰.

In some embodiments, R³ is selected from:

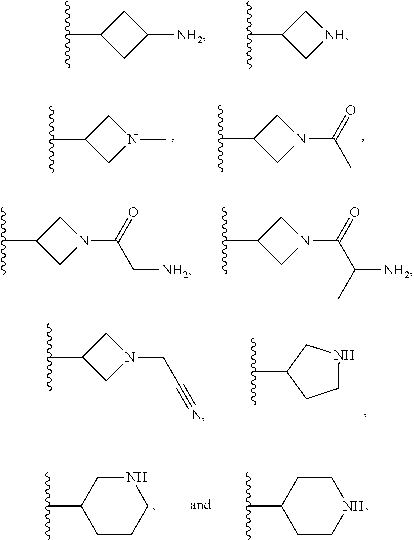

any of which is unsubstituted or substituted with one or more R¹⁰.

In some embodiments, R³ is selected from:

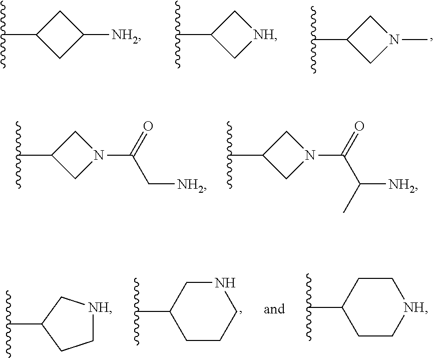

any of which is unsubstituted or substituted with one or more R¹⁰.

In some embodiments, R² and R³, together with the atom to which they are attached, form a monocyclic heterocycle that is unsubstituted or substituted with one or more R¹¹. In some embodiments, R² and R³, together with the atom to which they are attached, form a multicyclic heterocycle that is unsubstituted or substituted with one or more R¹¹. In some embodiments, R² and R³, together with the atom to which they are attached, form a 4-10 membered heterocycle that is unsubstituted or substituted with one or more R¹¹. In some embodiments, R² and R³, together with the atom to which they are attached, form a 4-6 membered heterocycle that is unsubstituted or substituted with one or more R¹¹. In some embodiments, R² and R³, together with the atom to which they are attached, form a structure that includes at least one nitrogen atom. In some embodiments, R² and R³, together with the atom to which they are attached, form a ring having the structure:

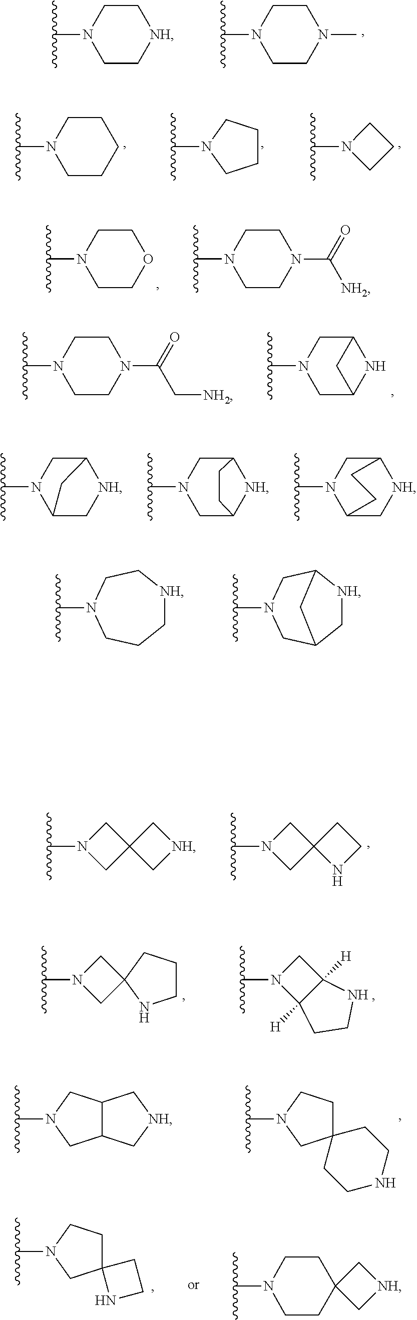

any of which is unsubstituted or substituted with one or more R¹¹. In some embodiments, R² and R³, together with the atom to which they are attached, form a ring having the structure:

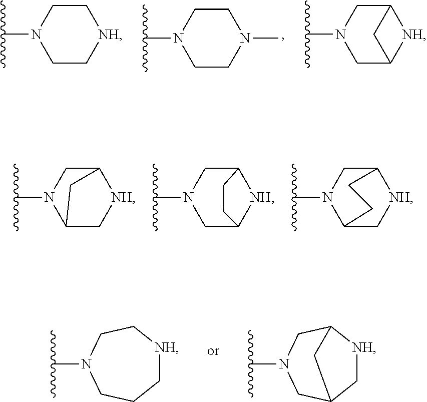

which ring is unsubstituted or substituted with one or more R¹¹. In some embodiments, R² and R³, together with the atom to which they are attached, form a ring having the structure:

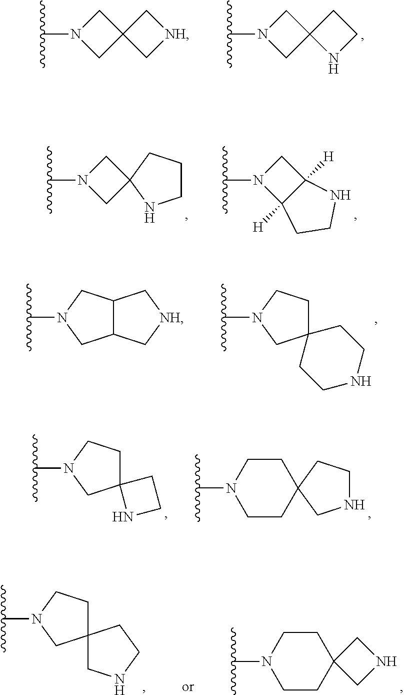

which ring is unsubstituted or substituted with one or more R¹¹. In some embodiments, R² and R³, together with the atom to which they are attached, form a ring having the structure:

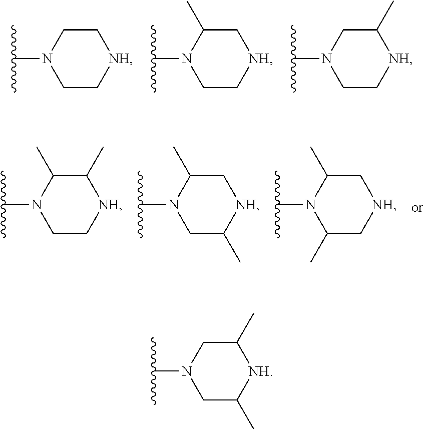

In some embodiments, R² and R³, together with the atom to which they are attached, form a heterocycle having the structure:

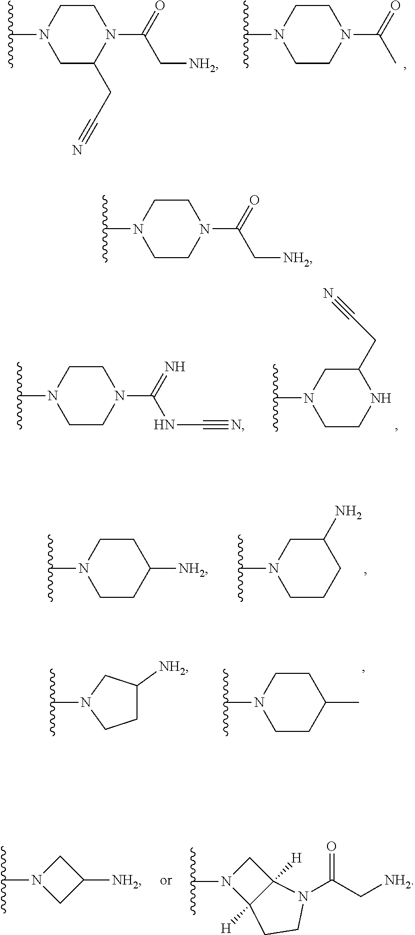

In some embodiments, R² and R³, together with the atom to which they are attached, form a 4-10 membered heterocycle that includes at least two nitrogen atoms (e.g., includes an amino moiety (e.g., —NR—)) and/or is substituted with a group including an amino moiety (e.g., —NRR′). In some embodiments, R² and R³, together with the atom to which they are attached, form a 4-6 membered heterocycle that includes at least two nitrogen atoms (e.g., includes an amino moiety (e.g., —NR—)) and/or is substituted with a group including an amino moiety (e.g., —NRR′).

In some embodiments, R⁴ is H. In some embodiments, R⁴ is a halogen. In some embodiments, R⁴ is —OR¹². In some embodiments, R⁴ is —OCH₃.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is selected from C₁₋₆alkyl that is unsubstituted or substituted with one or more R¹³. In some embodiments, R⁵ is selected from C₁₋₆alkyl that is unsubstituted, such as methyl or ethyl. In some embodiments, R⁵ is selected from C₁₋₆alkyl that is substituted with one or more halogens or —CN. In some embodiments, R⁵ is selected from —CF₃, —CF₂H, and —CH₂CN. In some embodiments, R⁵ is selected from —OR¹², wherein R¹² is selected from C₁₋₆alkyl that is unsubstituted or substituted with one or more R¹³. In some embodiments, R⁵ is —OCH₃, —OCF₃, or —OCF₂H.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, R⁶ is a bicyclic aryl substituted with one or more R¹⁵. In some embodiments, R⁶ is selected from:

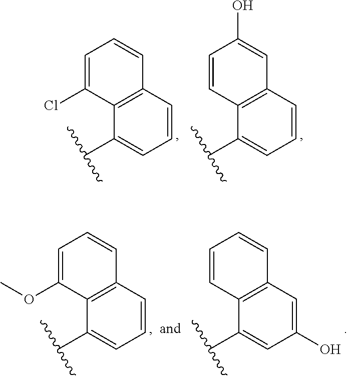

In some embodiments, R⁶ is a bicyclic heteroaryl substituted with one or more R¹⁵. In some embodiments, R⁶ is selected from:

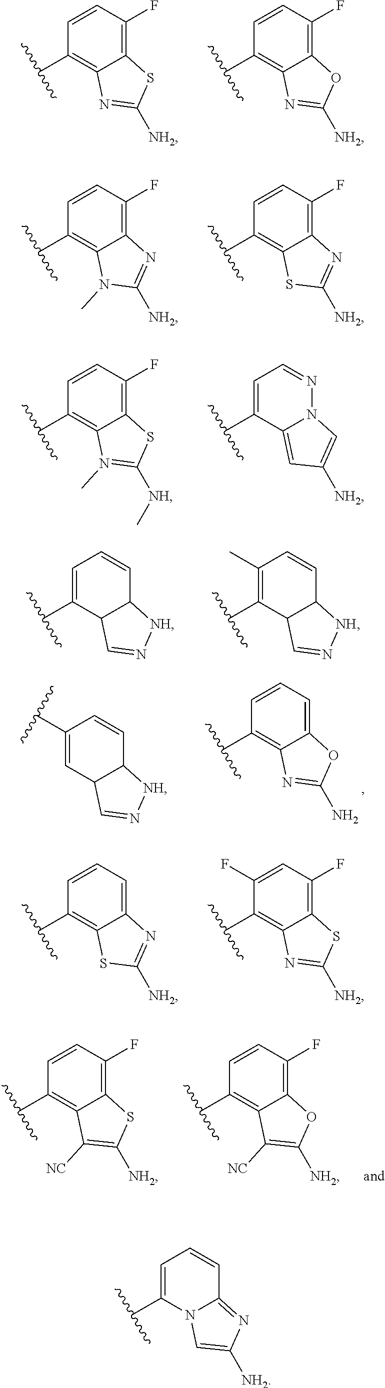

In some embodiments, R⁶ is selected from:

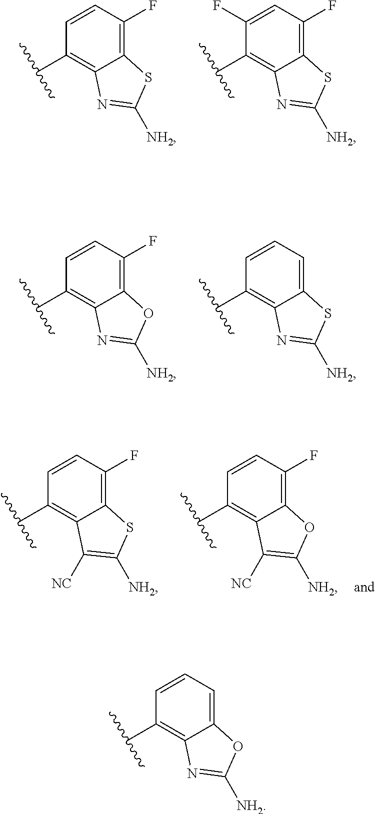

In some embodiments, R⁶ is selected from:

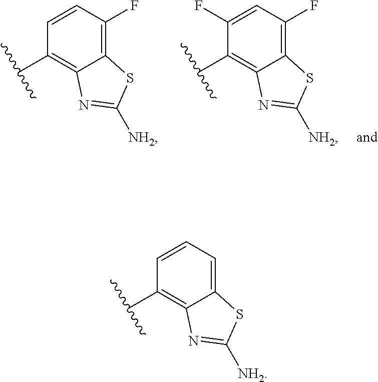 [SMILES: *c1ccc(F)c2sc(N)nc12 ; *c1c(F)cc(F)c2sc(N)nc12 ; *c1cccc2sc(N)nc12]

In some embodiments, (i) R⁶ is a bicyclic heteroaryl substituted with one or more R¹⁵ and (ii) R² and R³, together with the atom (e.g., nitrogen atom) to which they are attached, form a heterocycle that is unsubstituted or substituted with one or more R¹¹. In some embodiments, R⁶ is selected from:

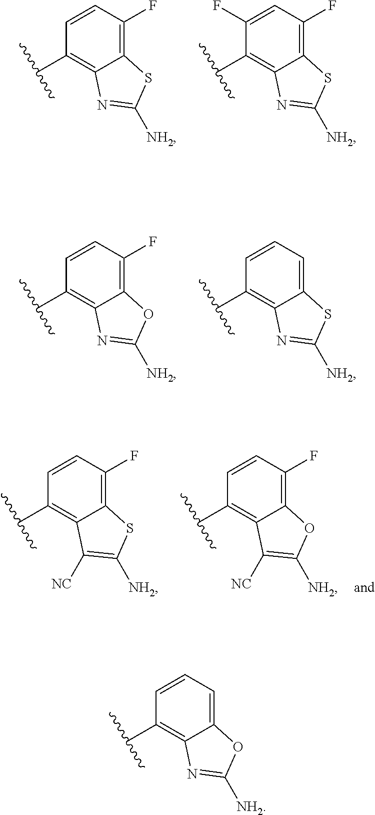

In some embodiments, R² and R³, together with the atom to which they are attached, form a heterocycle having the structure:

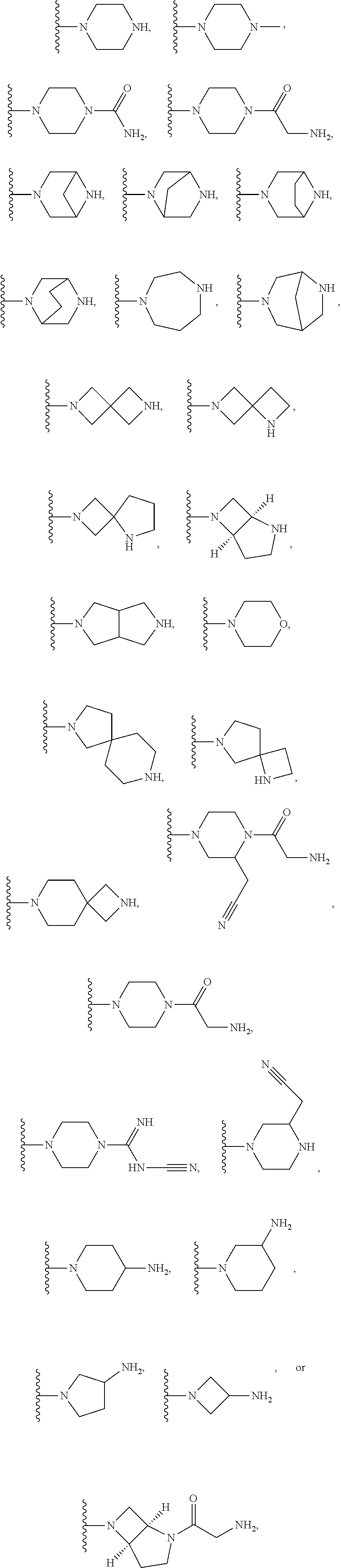

any of which is unsubstituted or substituted with one or more R¹¹. In some embodiments, R² and R³, together with the atom to which they are attached, form a heterocycle having the structure:

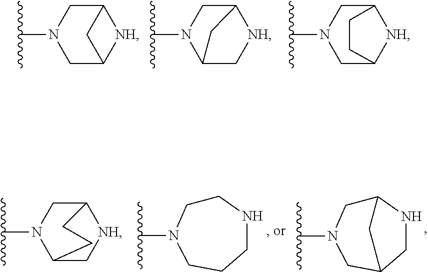

any of which is unsubstituted or substituted with one or more R¹¹. In some embodiments, R² and R³, together with the atom to which they are attached, form a heterocycle having the structure:

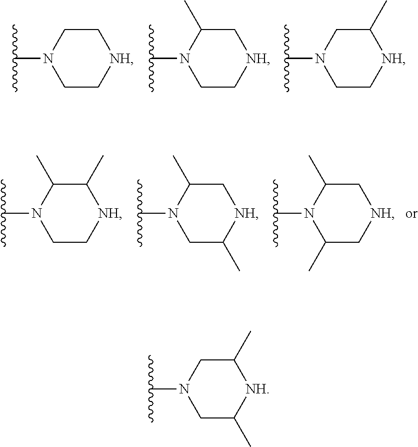

In some embodiments, R⁴ is H. In some embodiments, R⁷ is a halogen (e.g., F). In some embodiments, (i) R⁶ is a bicyclic heteroaryl substituted with one or more R¹⁵ and (ii) R² and R³, together with the atom (e.g., nitrogen atom) to which they are attached, form a bridged heterocyclic ring system that is unsubstituted or substituted with one or more R¹¹. In some embodiments, (i) R⁶ is a bicyclic heteroaryl substituted with one or more R¹⁵ and (ii) R² and R³, together with the atom (e.g., nitrogen atom) to which they are attached, form a bridged heterocyclic ring system comprising a piperazine ring, which bridged heterocyclic ring system is unsubstituted or substituted with one or more R¹¹. In some embodiments, (i) R⁶ is a bicyclic heteroaryl substituted with one or more R¹⁵ and (ii) R² and R³, together with the atom (e.g., nitrogen atom) to which they are attached, form a piperazine ring that is unsubstituted or substituted with one or more R¹¹.

In some embodiments, (i) R⁶ is a bicyclic heteroaryl substituted with one or more R¹⁵; (ii) R² is selected from H, C₁₋₆ alkyl, and a 3-6 membered carbocycle, wherein the C₁₋₆ alkyl or 3-6 membered carbocycle is unsubstituted or is substituted with one or more R¹³; and (iii) R³ is C₁₋₆ alkyl, which C₁₋₆alkyl is substituted with one or more R⁹. In some embodiments, R³ is C₁₋₆ alkyl substituted with —NH₂. In some embodiments, R² is H. In some embodiments, R² is C₁₋₆ alkyl that is unsubstituted or is substituted with one or more R¹³. In some embodiments, R² is a 3-6 membered carbocycle that is unsubstituted or is substituted with one or more R¹³.

In some embodiments, (i) R⁶ is a bicyclic heteroaryl substituted with one or more R¹⁵; (ii) R² is selected from H, C₁₋₆ alkyl, and a 3-6 membered carbocycle, wherein the C₁₋₆ alkyl or 3-6 membered carbocycle is unsubstituted or is substituted with one or more R¹³; and (iii) R³ is a carbocycle, which carbocycle is unsubstituted or substituted with one or more R¹⁰. In some embodiments, R³ is a cyclobutyl that is unsubstituted or substituted with one or more R¹⁰. In some embodiments, R³ is a carbocycle that is substituted with —N(R¹⁹)₂, wherein each R¹⁹ is independently selected from unsubstituted or substituted C₁₋₆ alkyl and H. In some embodiments, R³ is a carbocycle that is substituted with —NH₂. In some embodiments, R² is H. In some embodiments, R² is C₁₋₆ alkyl that is unsubstituted or is substituted with one or more R¹³. In some embodiments, R² is a 3-6 membered carbocycle that is unsubstituted or is substituted with one or more R¹³.

In some embodiments, (i) R⁶ is a bicyclic heteroaryl substituted with one or more R¹⁵; (ii) R² is selected from H, C₁₋₆ alkyl, and a 3-6 membered carbocycle, wherein the C₁₋₆ alkyl or 3-6 membered carbocycle is unsubstituted or is substituted with one or more R¹³; and (iii) R³ is a heterocycle, which heterocycle is unsubstituted or substituted with one or more R¹⁰. In some embodiments, the heterocycle includes one or more nitrogen atoms. In some embodiments, R³ is a heterocycle that is substituted with —N(R¹⁹)₂, wherein each R¹⁹ is independently selected from unsubstituted or substituted C₁₋₆ alkyl and H. In some embodiments, R³ is a heterocycle that is substituted with —NH₂. In some embodiments, R³ is a bridged heterocyclic ring system. In some embodiments, R³ is a monocycle. In some embodiments, R³ is an azetidine or a pyrrolidine that is unsubstituted or substituted with one or more R¹⁰. In some embodiments, R³ is selected from:

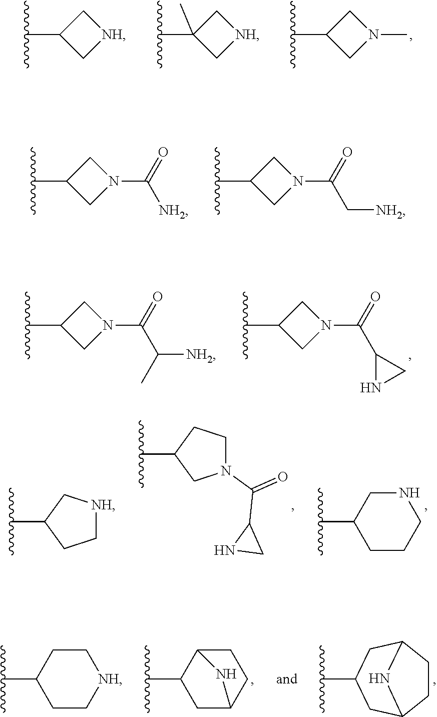

any of which is unsubstituted or substituted with one or more R¹⁰. In some embodiments, R² is H. In some embodiments, R² is C₁₋₆ alkyl that is unsubstituted or is substituted with one or more R¹³. In some embodiments, R² is a 3-6 membered carbocycle that is unsubstituted or is substituted with one or more R¹³.

In an aspect, the present disclosure provides a compound represented by Formula IF:

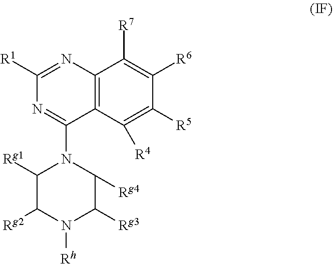

or a salt (e.g., pharmaceutically acceptable salt), ester, tautomer, prodrug, zwitterionic form, or stereoisomer thereof, wherein R¹, R⁴, R⁵, R⁶, and R⁷ are as defined above for Formula I and described in classes and subclasses herein, both singly and in combination; R^{g1}, R^{g2}, R^{g3}, and R^{g4} are each independently selected from H and C₁₋₆alkyl; or (a) R^{g1} and R^{g3} or (b) R^{g2} and R^{g3} join together to form a second ring containing 4-6 members, and any of R^{g1}, R^{g2}, R^{g3}, and R^{g4} that are not part of the second ring are independently selected from H and C₁₋₆alkyl, wherein any C₁₋₆alkyl is unsubstituted or substituted with one or more R¹³; and R^{h} is selected from H, C₁₋₆alkyl, —C(O)NH₂, and —C(O)C₁₋₆alkylNH₂. In some embodiments, the present disclosure provides a compound of Formula IF, or a salt (e.g., a pharmaceutically acceptable salt) thereof.

In some embodiments, the present disclosure provides a compound of Formula IF, wherein:
R¹ is selected from —OR⁸, a 4-6 membered heterocycle including a nitrogen atom, and H, wherein the heterocycle is unsubstituted or substituted with one or more R¹⁶;
R^{g1}, R^{g2}, R^{g3}, and R^{g4} are each independently selected from H and C₁₋₆alkyl; or (a) R^{g1} and R^{g3} or (b) R^{g2} and R^{g3} join together to form a second ring containing 4-6 members, and any of R^{g1}, R^{g2}, R^{g3}, and R^{g4} that are not part of the second ring are independently selected from H and C₁₋₆alkyl, wherein any C₁₋₆alkyl is unsubstituted or substituted with one or more R¹³;
R^{h} is selected from H, C₁₋₆alkyl, —C(O)NH₂, and —C(O)C₁₋₆alkylNH₂;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³; and
each R²² is independently selected from C₁₋₆ alkyl, C₂₋₆ alkenyl, and H.

In some embodiments, R¹ is —OR⁸. In some embodiments, R⁸ is a heterocycle. In some embodiments, R⁸ is an alkylheterocycle. In some embodiments, a heterocycle or a heterocycle of an alkylheterocycle comprises 4-8 members including at least one heteroatom selected from N, O, and S. In some embodiments, R⁸ comprises a heterocycle including at least one nitrogen atom. In some embodiments, R⁸ comprises a 4-8 membered heterocycle including at least one nitrogen atom. In some embodiments, the heterocycle comprises one or more R¹⁶ substituents. In some embodiments, at least one R¹⁶ is —OR¹², wherein R¹² is independently selected from C₁₋₆ alkyl, C₂₋₆ alkenyl, and H. In some embodiments, at least one R¹⁶ is —OCH₃.

In some embodiments, R¹ is selected from:

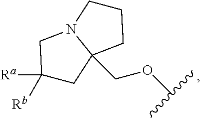

wherein R^{a} and R^{b} are each independently selected from halogen, C₁₋₆alkyl, —OR¹², and H. In some embodiments, R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R¹ is selected from:

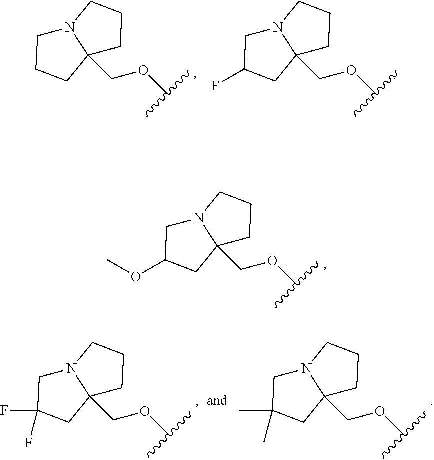 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, R¹ is selected from:

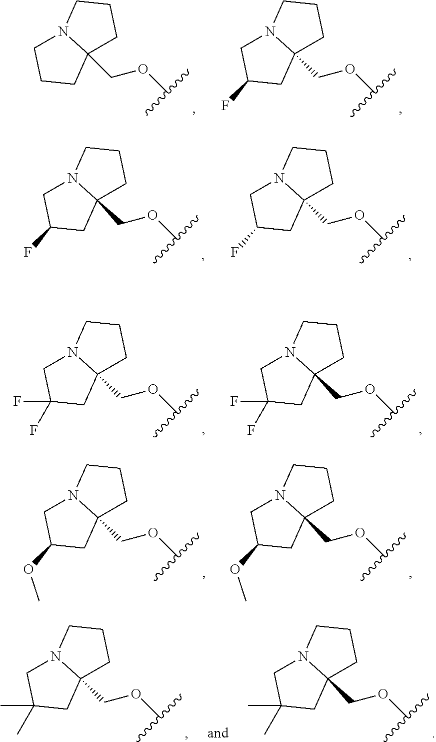

In some embodiments, R¹ is selected from:

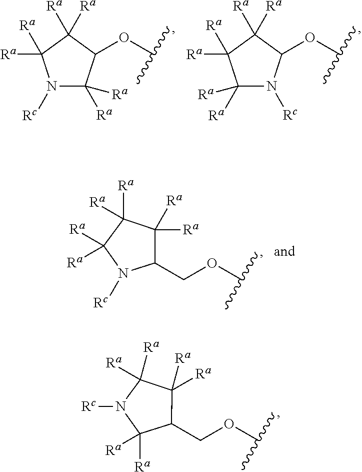

wherein each R^{a} is independently selected from halogen, C₁₋₆ alkyl, —OR¹², and H; and wherein R^{c} is selected from C₁₋₆ alkyl, wherein a C₁₋₆ alkyl of a R^{a} or R^{c} is unsubstituted or is substituted with one or more R¹³. In some embodiments, R¹ is selected from:

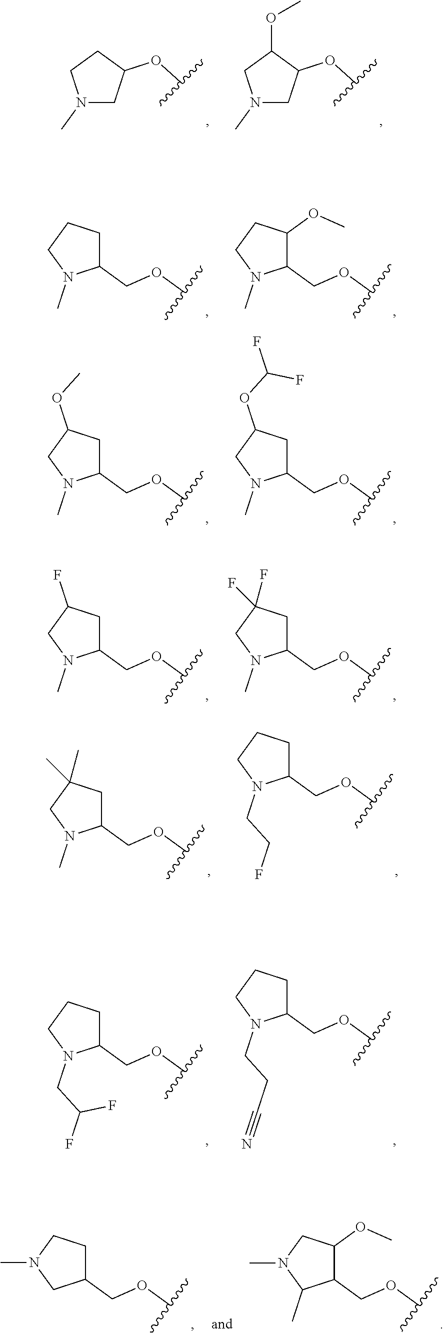

In some embodiments, R¹ is selected from:

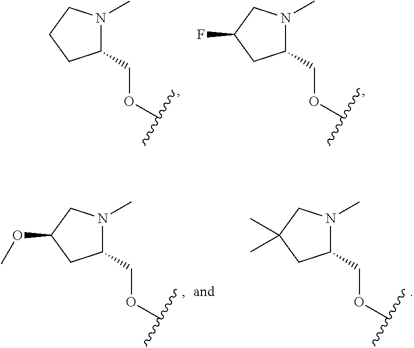

In some embodiments, R¹ is selected from:

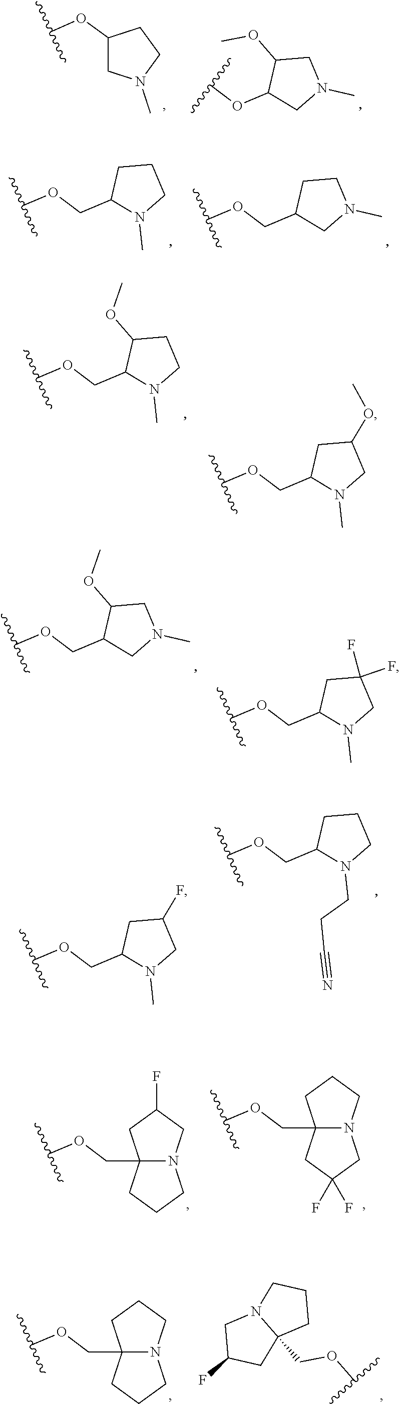

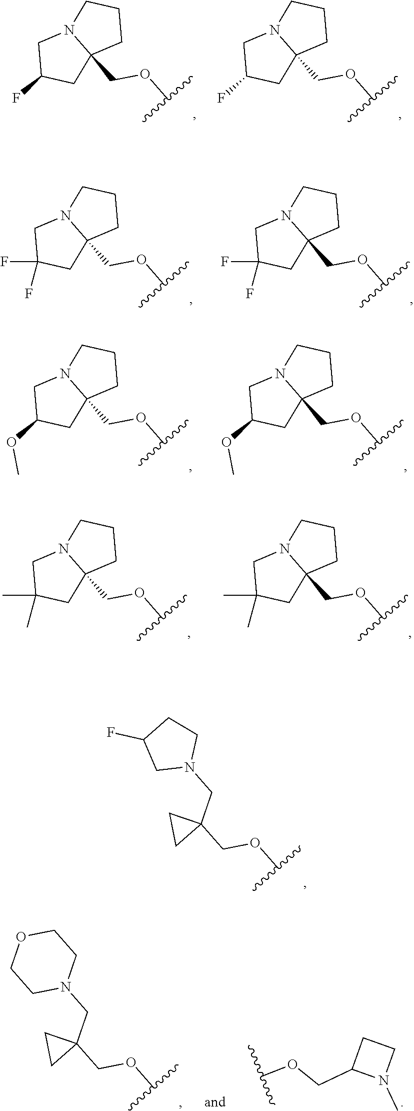

In some embodiments, R¹ is selected from.

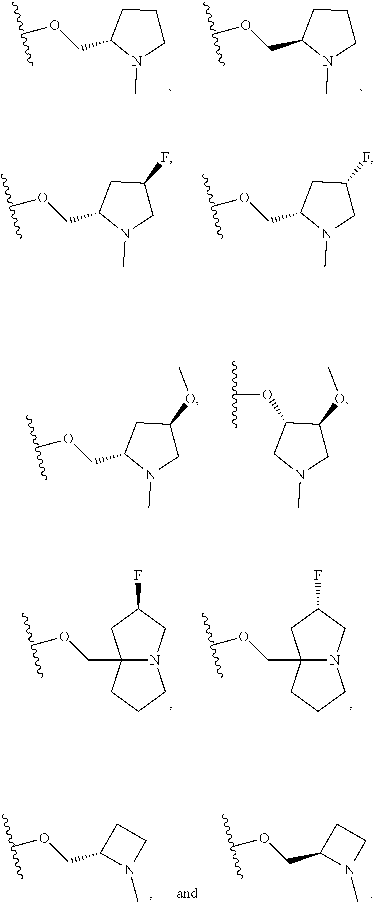

In some embodiments, R¹ is a 4-6 membered heterocycle including a nitrogen atom, which heterocycle is unsubstituted or substituted with one or more R¹⁶. In some embodiments, R¹ is selected from:

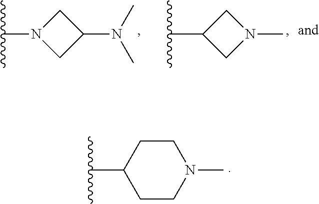

In some embodiments, R¹ is H.

In some embodiments, R⁴ is H. In some embodiments, R⁴ is a halogen. In some embodiments, R⁴ is —OR¹². In some embodiments, R⁴ is —OCH₃.

In some embodiments, R⁵ is a 3-6 membered carbocycle. In some embodiments, R⁵ is a 3-6 membered heterocycle. In some embodiments, R⁵ is a 5 or 6 membered aryl or heteroaryl moiety. In some embodiments, R⁵ is a furan.

In some embodiments, R⁵ is selected from C₁₋₆alkyl that is unsubstituted or substituted with one or more R¹³. In some embodiments, R⁵ is selected from C₁₋₆alkyl that is unsubstituted, such as methyl or ethyl. In some embodiments, R⁵ is selected from C₁₋₆alkyl that is substituted with one or more halogens or —CN. In some embodiments, R⁵ is selected from —CF₃, —CF₂H, and —CH₂CN. In some embodiments, R⁵ is selected from —OR¹², wherein R¹² is selected from C₁₋₆alkyl that is unsubstituted or substituted with one or more R¹³. In some embodiments, R⁵ is —OCH₃, —OCF₃, or —OCF₂H.

In some embodiments, R⁵ is a halogen. In some embodiments, R⁵ is Cl or F. In some embodiments, R⁷ is a halogen. In some embodiments, R⁷ is Cl or F. In some embodiments, R⁵ and R⁷ are both halogens. In some embodiments, R⁵ and R⁷ are both selected from Cl and F. In some embodiments, R⁴, R⁵, and R⁷ are each independently halogens. In some embodiments, R⁴, R⁵, and R⁷ are each selected from Cl and F.

In some embodiments, R⁶ is a bicyclic aryl substituted with one or more R¹⁵. In some embodiments, R⁶ is selected from:

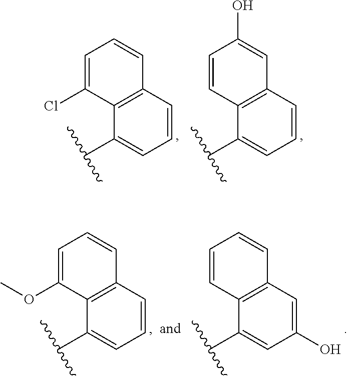

In some embodiments, R⁶ is a bicyclic heteroaryl substituted with one or more R¹⁵. In some embodiments, R⁶ is selected from:

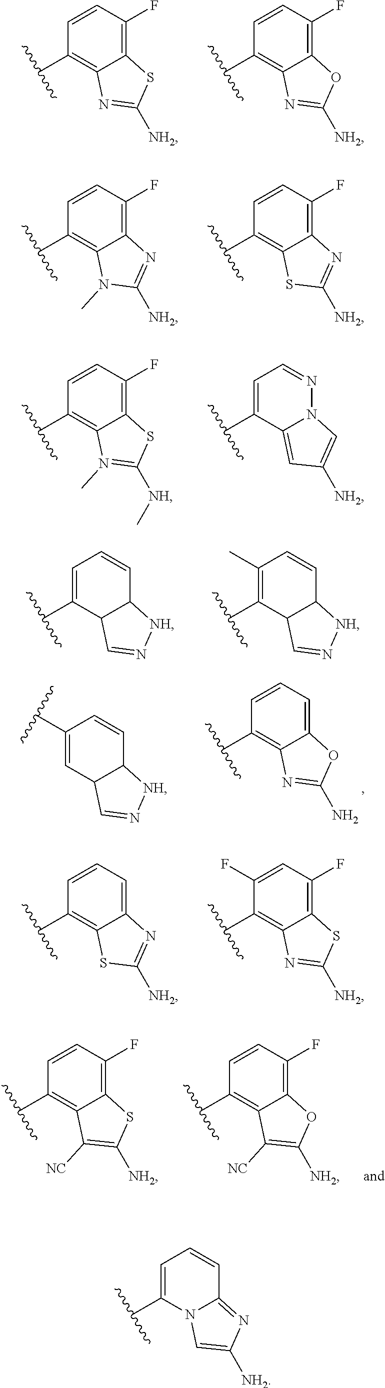

In some embodiments, R⁶ is selected from:

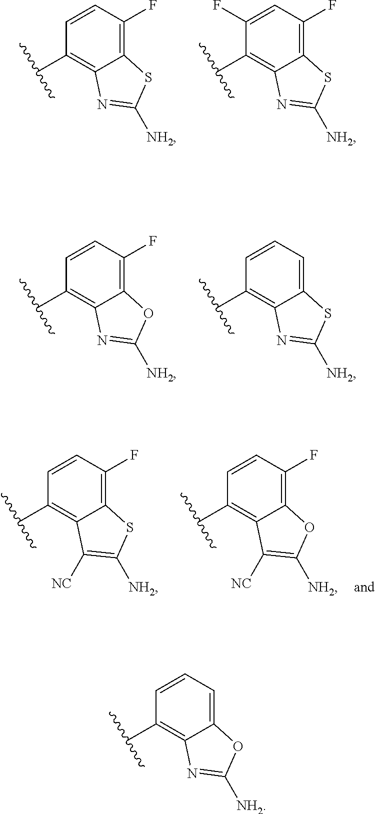

In some embodiments, R⁶ is selected from:

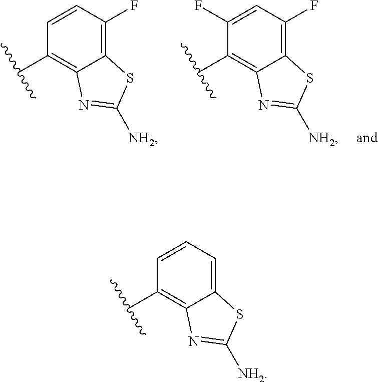 [SMILES: *c1ccc(F)c2sc(N)nc12 ; *c1c(F)cc(F)c2sc(N)nc12 ; *c1cccc2sc(N)nc12]

In some embodiments, R^{g1} is selected from C₁₋₆alkyl (e.g., methyl). In some embodiments, R^{g2} is selected from C₁₋₆alkyl (e.g., methyl). In some embodiments, R^{g3} is selected from C₁₋₆alkyl (e.g., methyl). In some embodiments, R^{g4} is selected from C₁₋₆alkyl (e.g., methyl). In some embodiments, R^{g1} and R^{g2} are selected from C₁₋₆alkyl (e.g., methyl). In some embodiments, R^{g1} and R^{g2} are both methyl. In some embodiments, R^{g1} and R^{g3} are selected from C₁₋₆alkyl (e.g., methyl). In some embodiments, R^{g1} and R^{g3} are both methyl. In some embodiments, R^{g1} and R^{g4} are selected from C₁₋₆alkyl (e.g., methyl). In some embodiments, R^{g1} and R^{g4} are both methyl. In some embodiments, R^{g2} and R^{g3} are selected from C₁₋₆alkyl (e.g., methyl). In some embodiments, R^{g2} and R^{g3} are both methyl.

In some embodiments, R^{g1} and R^{g3} join together to form a second ring containing 4-6 members. In some embodiments, R^{g2} and R^{g3} join together to form a second ring containing 4-6 members. In some embodiments, R^{h} is H.

In some embodiments, the compound has the structure:

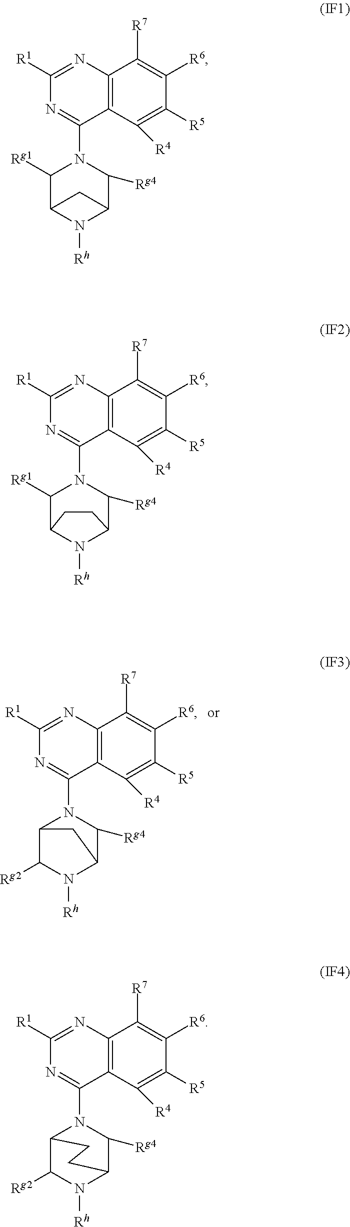

In some embodiments for a compound according to formula (IF1) or (IF2), R^{g1} and R^{g4} are each H. In some embodiments for a compound according to formula (IF3) or (IF4), R^{g2} and R^{g4} are each H. In some embodiments for a compound according to formula (IF1), (IF2), (IF3), or (IF4), R^{h} is H. In some embodiments for a compound according to formula (IF1), (IF2), (IF3), or (IF4), R¹ is selected from:

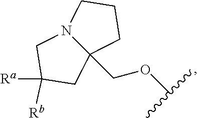

wherein R^{a} and R^{b} are each independently selected from halogen, C₁₋₆alkyl, —OR¹², and H, wherein any C₁₋₆alkyl is unsubstituted or is substituted with one or more R¹³. In some embodiments for a compound according to formula (IF1), (IF2), (IF3), or (IF4), R⁶ is selected from:

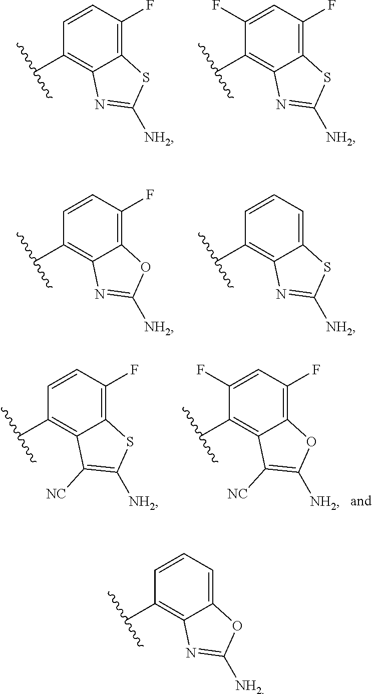

In some embodiments for a compound according to formula (IF1), (IF2), (IF3), or (IF4), R⁴ is H. In some embodiments for a compound according to formula (IF1), (IF2), (IF3), or (IF4), R⁷ is a halogen (e.g., F). In some embodiments for a compound according to formula (IF1), (IF2), (IF3), or (IF4), R⁵ is a halogen, —CN, a 3-6 membered carbocycle, a 3-6 membered heterocycle, or a C₁₋₆alkyl that is unsubstituted or substituted with one or more R¹³.

In another aspect, the present disclosure provides a compound according to Formula II:

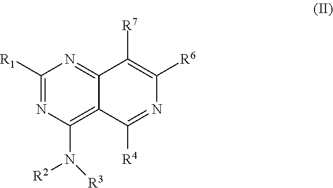

or a salt (e.g., pharmaceutically acceptable salt), ester, tautomer, prodrug, zwitterionic form, or stereoisomer thereof, wherein:
R¹ is selected from —OR⁸, a 4-6 membered heterocycle including a nitrogen atom, and H, wherein the heterocycle is unsubstituted or substituted with one or more R¹⁶;
R² is selected from H, a 3-6 membered carbocycle, and C₁₋₆ alkyl, wherein the 3-6 membered carbocycle or the C₁₋₆ alkyl is unsubstituted or substituted with one or more R¹³;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a phenyl, monocyclic heteroaryl, bicyclic aryl, or bicyclic heteroaryl, wherein any phenyl, monocyclic heteroaryl, bicyclic aryl, and bicyclic heteroaryl is unsubstituted or substituted with one or more R¹⁵;
R⁷ is selected from halogen, —OR¹², —CN, and H;
R⁸ is selected from H, heterocycle, and alkylheterocycle, wherein the heterocycle or alkylheterocycle is unsubstituted or substituted with one or more R¹⁶, and wherein an alkyl moiety of any alkylheterocycle is selected from C₁₋₆ alkyl;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from halogen, —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²¹;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, —OR¹², —CN, —N(R¹²)₂, and C₁₋₆alkyl, wherein any C₁₋₆alkyl is unsubstituted or substituted with one or more R¹³;
each R¹⁵ is independently selected from halogen, —OR¹², —CN, —N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, —OR¹², and 3-6 membered heterocycle, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl, 3-6 membered heterocycle, and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, halogen, and 3-6 membered carbocycle;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, the present disclosure provides a compound of Formula II, or a salt (e.g., a pharmaceutically acceptable salt) thereof.

In some embodiments, the present disclosure provides a compound of Formula II, wherein:
R¹ is selected from —OR⁸, a 4-6 membered heterocycle including a nitrogen atom, and H, wherein a heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰; each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, —OR¹², —CN, —N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

In some embodiments, R³ is selected from a carbocycle and a heterocycle, wherein the carbocycle or heterocycle is unsubstituted or substituted with one or more R¹⁰. In some embodiments, R³ is a 3-6 membered carbocycle or heterocycle that is unsubstituted or substituted with one or more R¹⁰. In some embodiments, R³ is a 4-6 membered carbocycle, which carbocycle is unsubstituted or substituted with one or more R¹⁰. In some embodiments, R³ is a 4-8 membered heterocycle having 1-2 heteroatoms independently selected from nitrogen, oxygen, and sulfur, which heterocycle is unsubstituted or substituted with one or more R¹⁰. In some embodiments, R³ is azetidine unsubstituted or substituted with one or more R¹⁰. In some embodiments, R³ is selected from:

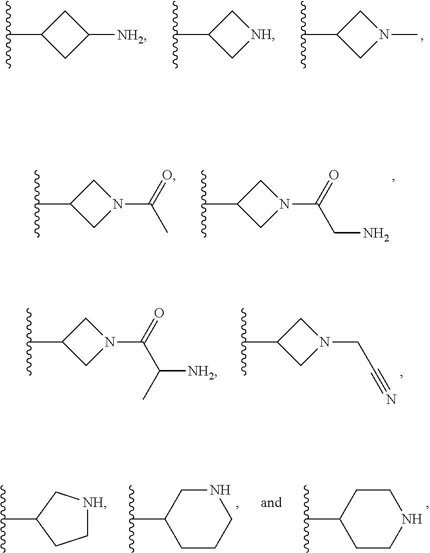

any of which is unsubstituted or substituted with one or more R¹⁰.

In some embodiments, R³ is selected from C₁₋₆ alkyl that is substituted with one or more R⁹. In some embodiments, R³ is selected from C₁₋₆alkyl-N(R¹⁷)₂. In some embodiments, R³ is —CH₂CH₂NH₂. In some embodiments, R³ is selected from:

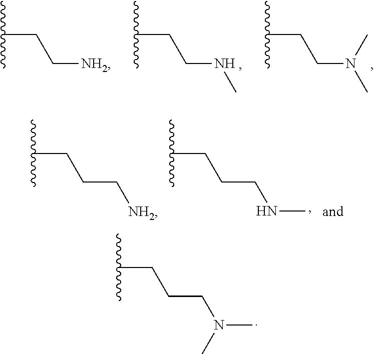

In some embodiments, R² or R³ includes an amino moiety (e.g., —NRR′). In some embodiments, R² or R³ is substituted with an amino moiety (i.e., —N(R¹⁷)₂ or —N(R¹⁹)₂).

In some embodiments, R² and R³, together with the atom to which they are attached, form a 4-6 membered heterocycle that is unsubstituted or substituted with one or more R¹¹. In some embodiments, R² and R³, together with the atom (e.g., nitrogen atom) to which they are attached, form a 4-9 membered heterocycle having 0-2 additional heteroatoms independently selected from nitrogen, oxygen, and sulfur, wherein the heterocycle is unsubstituted or substituted with one or more R¹¹. In some embodiments, R² and R³, together with the atom (e.g., nitrogen atom) to which they are attached, form a 4-7 membered monocyclic heterocycle having 0-1 additional heteroatoms independently selected from nitrogen, oxygen, and sulfur, wherein the heterocycle is unsubstituted or substituted with one or more R¹¹. In some embodiments, R² and R³, together with the atom (e.g., nitrogen atom) to which they are attached, form a piperazine. In some embodiments, R² and R³, together with the atom (e.g., nitrogen atom) to which they are attached, form a 7-9 membered bicyclic heterocycle having 0-2 additional heteroatoms independently selected from nitrogen, oxygen, and sulfur, wherein the heterocycle is unsubstituted or substituted with one or more R¹¹. In some embodiments, R² and R³, together with the atom to which they are attached, form a heterocycle having the structure:

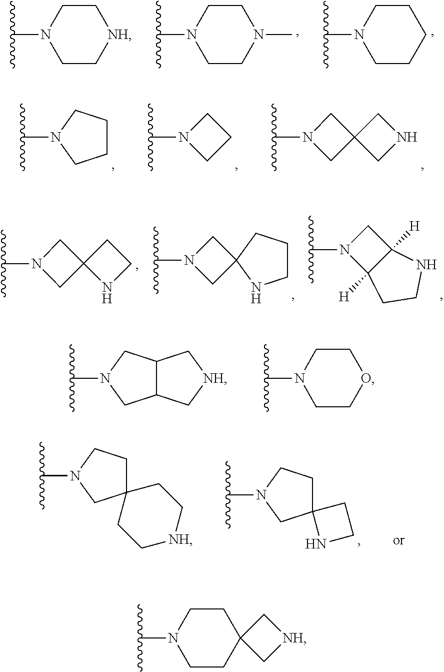

which ring is unsubstituted or substituted with one or more R¹¹. In some embodiments, R² and R³, together with the atom to which they are attached, form a heterocycle having the structure:

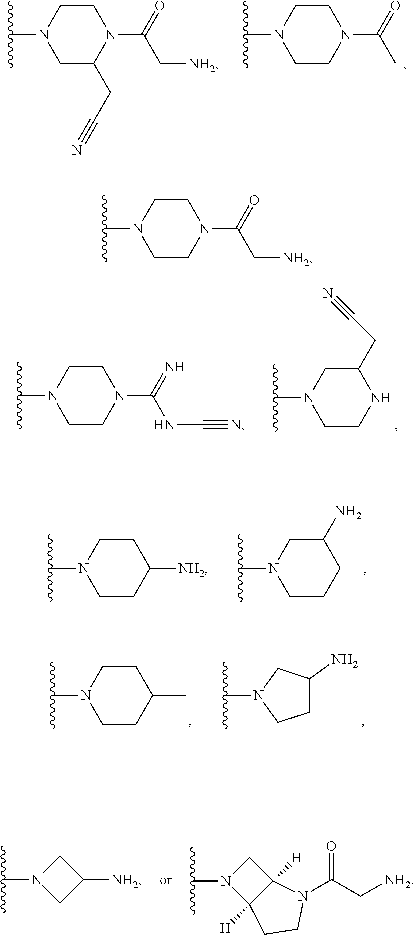

In some embodiments, R² and R³, together with the nitrogen atom to which they are attached, form a heterocycle that is unsubstituted or substituted with one or more R¹¹, which heterocycle includes an additional nitrogen atom (e.g., an amino moiety (e.g., —NR—)) and/or is substituted with a group including an amino moiety (e.g., —N(R¹⁹)₂).

In some embodiments, R¹ is H.

In some embodiments, R¹ is —OR⁸. In some embodiments, R¹ is —OR⁸, wherein R⁸ is a heterocycle or an alkylheterocycle. In some embodiments, R⁸ comprises a 3-6 membered heterocycle that is unsubstituted or substituted with one or more R¹⁴. In some embodiments, R⁸ is a heterocycle. In some embodiments, R⁸ is an alkylheterocycle. In some embodiments, R⁸ is an alkylheterocycle, wherein the alkyl moiety of the alkylheterocycle is selected from C₁₋₆ alkyl. In some embodiments, R⁸ is —CH₂(heterocycle). In some embodiments, a heterocycle or a heterocycle of an alkylheterocycle comprises 4-8 members including at least one heteroatom selected from N, O, and S. In some embodiments, R⁸ comprises a heterocycle including at least one nitrogen atom. In some embodiments, R⁸ comprises a 4-8 membered heterocycle including at least one nitrogen atom. In some embodiments, a heterocycle or a heterocycle of an alkylheterocycle is a 4-6 monocyclic heterocycle having 1-2 heteroatoms independently selected from nitrogen, oxygen, and sulfur. In some embodiments, a heterocycle or a heterocycle of an alkylheterocycle is an 8-membered bicyclic heterocycle having 1-2 heteroatoms independently selected from nitrogen, oxygen, and sulfur. In some embodiments, the heterocycle is substituted with one or more R¹⁶. In some embodiments, at least one R¹⁶ is —OR¹², wherein R¹² is independently selected from C₁₋₆ alkyl, C₂₋₆ alkenyl, and H. In some embodiments, at least one R¹⁶ is —OCH₃. In some embodiments, at least one R¹⁶ is halogen (e.g., F). In some embodiments, at least one R¹⁶ is C₁₋₆alkyl unsubstituted or substituted with one or more R¹³.

In some embodiments, R¹ is selected from:

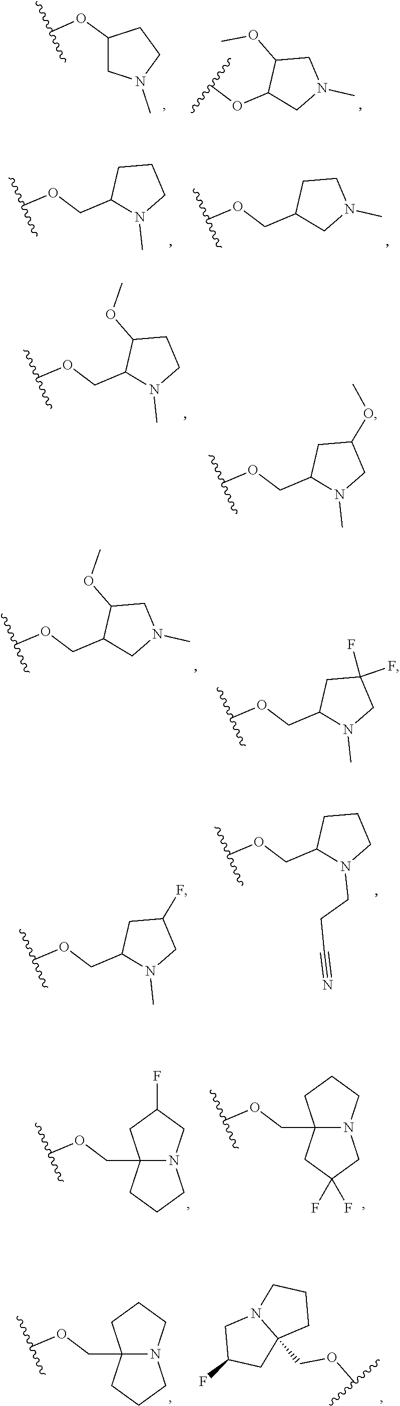

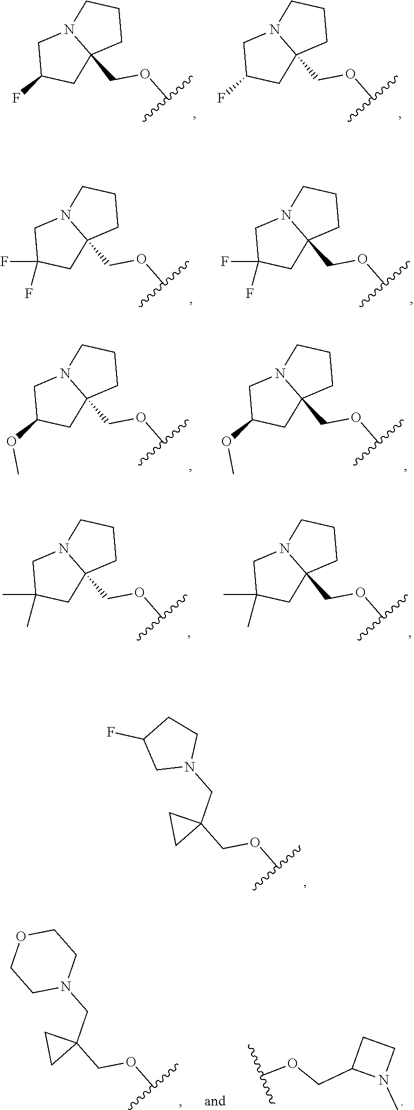

In some embodiments, R¹ is selected from.

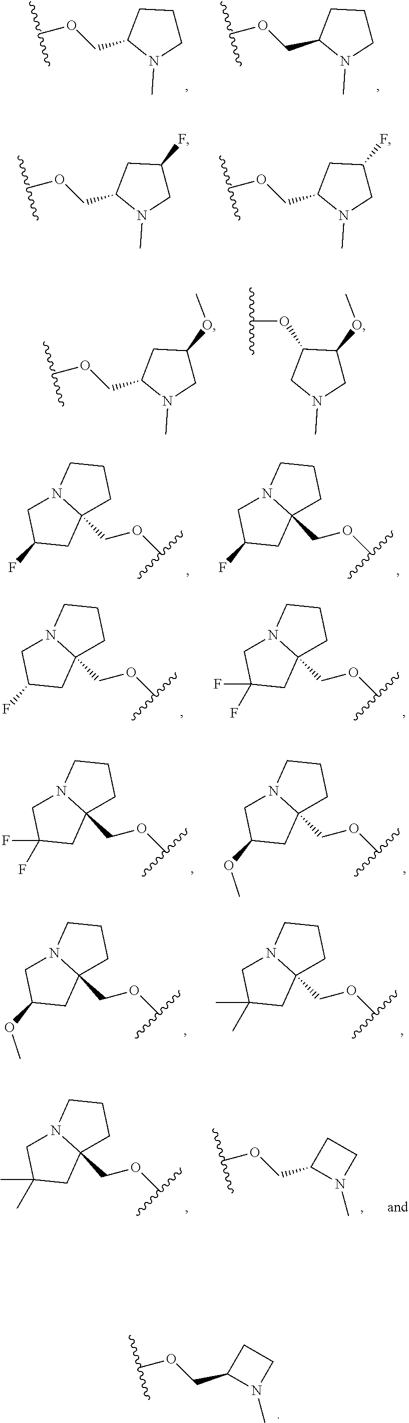

In some embodiments, R¹ is a 4-6 membered heterocycle including a nitrogen atom that is unsubstituted or substituted with one or more R¹⁶. In some such embodiments, R¹⁶ is selected from —N(R¹²)₂, C₁₋₆alkyl, and 3-6 membered heterocycle. In some embodiments, R¹⁶ is —N(C₁₋₆alkyl)₂, e.g., —N(CH₃)₂. In some embodiments, R¹⁶ is C₁₋₆alkyl (e.g., methyl). In some embodiments, R¹⁶ is a bicyclic 6-membered heterocycle having 1 nitrogen atom. In some embodiments, R¹ is selected from:

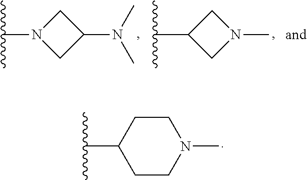

In some embodiments, R⁶ is a bicyclic aryl substituted with one or more R¹⁵. In some embodiments, R⁶ is naphthyl substituted with one or more R¹⁵.

In some embodiments, R⁶ is a 9-10 membered heteroaryl having 1-3 heteroatoms independently selected from nitrogen, oxygen, and sulfur that is unsubstituted or substituted with one or more R¹⁵. In some embodiments, R⁶ is a bicyclic heteroaryl substituted with one or more R¹⁵. In some embodiments, R⁶ is a 9-membered heteroaryl having 1-3 heteroatoms independently selected from nitrogen, oxygen, and sulfur and substituted with one or more R¹⁵. In some such embodiments, at least one R¹⁵ is —N(R¹²)₂ (e.g., —NH₂). In some embodiments, at least one R¹⁵ is a halogen (e.g., F). In some embodiments, each R¹⁵ is independently selected from halogen, —CN, and —N(R¹²)₂. In some embodiments, R⁶ is substituted with at least two R¹⁵ (e.g., at least a halogen and —NH₂).

In some embodiments, R⁶ is selected from:

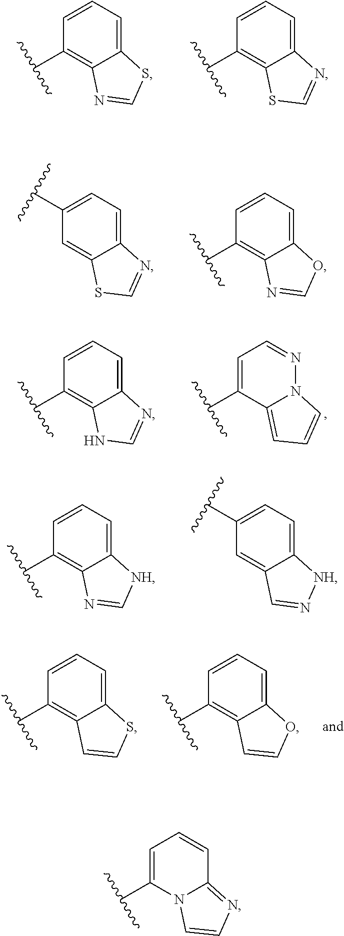

any of which is unsubstituted or substituted with one or more R¹⁵.

In some embodiments, R⁶ is selected from:

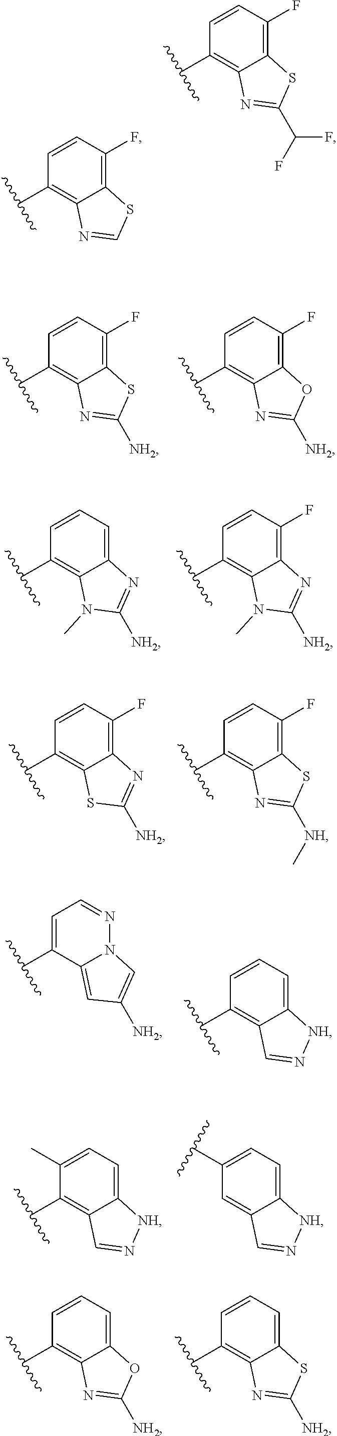

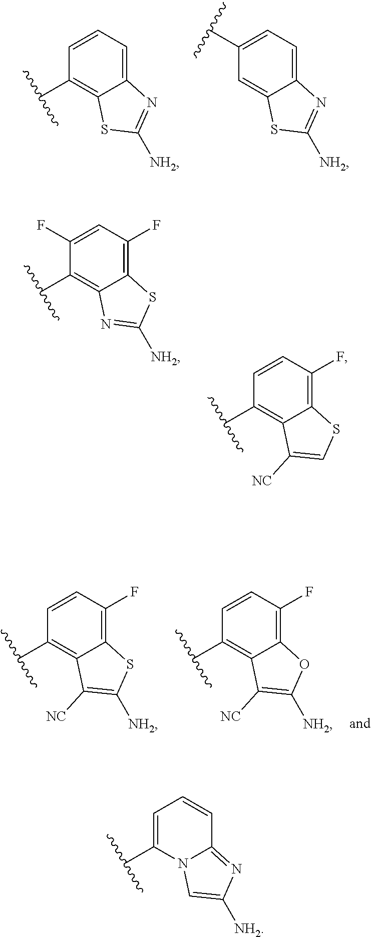

In some embodiments, R⁶ is selected from:

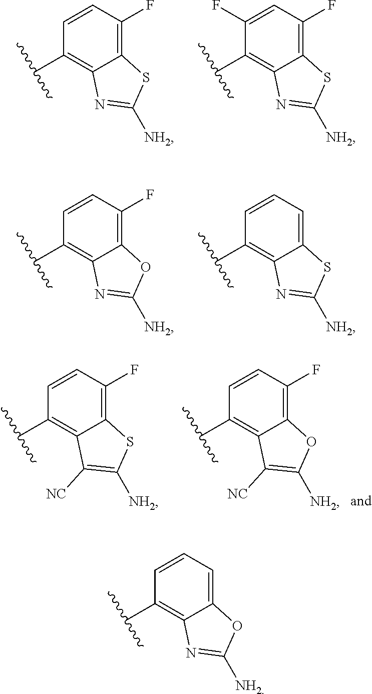

In some embodiments, R⁶ is:

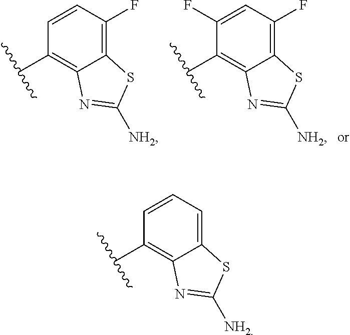 [SMILES: *c1ccc(F)c2sc(N)nc12 ; *c1c(F)cc(F)c2sc(N)nc12 ; "or" ; *c1cccc2sc(N)nc12]

In some embodiments, R⁶ is phenyl unsubstituted or substituted with one or more R¹⁵. In some such embodiments, each R¹⁵ is independently selected from halogen, —OR¹², —CN, and —N(R¹²)₂.

In some embodiments, R⁶ is a monocyclic 5-6 membered heteroaryl unsubstituted or substituted with one or more R¹⁵. In some embodiments, R⁶ is pyridyl unsubstituted or substituted with one or more R¹⁵. In some such embodiments, each R¹⁵ is independently selected from —N(R¹²)₂ and C₁₋₆alkyl, wherein any C₁₋₆alkyl is unsubstituted or substituted with one or more R¹³.

In some embodiments, R⁴ is H. In some embodiments, R⁴ is a halogen. In some embodiments, R⁴ is —OR¹². In some embodiments, R⁴ is —OCH₃.

In some embodiments, R⁷ is a halogen. In some embodiments, R⁷ is Cl or F. In some embodiments, R⁷ is —OR¹², wherein R¹² is selected from H and C₁₋₆alkyl that is unsubstituted or substituted with one or more R¹³. In some embodiments, R⁷ is —OH, —OCH₃, or —OCH₂CF₃. In some embodiments, R⁷ is —CN. In some embodiments, R⁷ is hydrogen.

In some embodiments, R⁴ and R⁷ are both halogens. In some embodiments, R⁴ and R⁷ are both selected from Cl and F.

In some embodiments, the compound is represented by a formula included in any of Tables 2-9. In some embodiments, the present disclosure provides a compound selected from any one of Tables 2, 3, 4, 5, 7, 8, and 9 or a salt (e.g., a pharmaceutically acceptable salt thereof). In some embodiments, the present disclosure provides a compound selected from any one of Tables 2, 3, 4, 7, 8, and 9 or a salt (e.g., a pharmaceutically acceptable salt thereof).

Also provided herein is a compound selected from any of Tables 2-9 or any of the Examples provided herein, or a salt, ester, tautomer, prodrug, zwitterionic form, or stereoisomer thereof. It will be appreciated that compounds described herein may be provided and/or utilized in any available form (e.g., a salt form) and that all such forms are contemplated by the present disclosure. The present disclosure also contemplates forms such as esters, tautomers, prodrugs, zwitterionic forms, and stereoisomers of the compounds provided herein.

In some embodiments, provided compounds are provided and/or utilized in a salt form (e.g., a pharmaceutically acceptable salt form). Reference to a compound provided herein is understood to include reference to salts thereof, unless otherwise indicated.

Also provided herein are embodiments wherein any embodiment described herein may be combined with any one or more of these embodiments, provided the combination is not mutually exclusive. As used herein, two embodiments are “mutually exclusive” when one is defined to be something which is different than the other. For example, an embodiment wherein two groups combine to form a ring is mutually exclusive with an embodiment in which one group is ethyl and the other group is hydrogen. Similarly, an embodiment wherein one group is CH₂ is mutually exclusive with an embodiment wherein the same group is NH.

### Compositions

The present disclosure also provides a composition (e.g., a pharmaceutical composition) comprising a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof. In some embodiments, a provided composition comprises a compound provided herein, or a pharmaceutically acceptable salt thereof. For example, the present disclosure provides a pharmaceutical composition comprising a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, together with a pharmaceutically acceptable carrier. In some embodiments, a provided pharmaceutical composition comprises a compound provided herein or a pharmaceutically acceptable salt thereof, together with a pharmaceutically acceptable carrier.

In some embodiments, the pharmaceutical composition is formulated for oral administration. In some embodiments, the oral pharmaceutical formulation is selected from a tablet and a capsule.

In some embodiments, the pharmaceutical composition is formulated for parenteral administration. In some embodiments, the pharmaceutical composition is formulated for intravenous administration. In some embodiments, the pharmaceutical composition is formulated for subcutaneous administration.

While it may be possible for certain compounds provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, to be administered as the raw chemical, compounds may additionally or alternatively be provided in a pharmaceutical formulation. Accordingly, provided herein are pharmaceutical formulations which comprise one or more compounds disclosed herein (e.g., a compound of any one of Formulas I, IA, IA1, IA2, IB, IB1, IB2, IC, ID, ID′, IE, IF, or II), or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration selected. Any of the well-known techniques, carriers, and excipients may be used as suitable and as understood in the art. The pharmaceutical compositions disclosed herein may be manufactured in any suitable manner known, e.g., by means of conventional mixing, dissolving, granulating, dragee-making, levigating, emulsifying, encapsulating, entrapping or compression processes.

A pharmaceutical formulation provided herein can be suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration. The most suitable route may depend on, for example, the condition and disorder of the subject to which the pharmaceutical formulation will be administered. A pharmaceutical formulation can be provided in a unit dosage form. A pharmaceutical formulation can be prepared by any suitable method. A method of preparing a pharmaceutical formulation may comprise bringing a compound provided herein (e.g., a compound of any one of Formulas I, IA, IA1, IA2, IB, IB1, IB2, IC, ID, ID′, IE, IF, or II), or a pharmaceutically acceptable salt, ester, amide, prodrug or solvate thereof (“active ingredient”) in contact with one or more pharmaceutically acceptable carriers (e.g., accessory ingredients). In general, the formulations are prepared by uniformly and intimately bringing into association the active ingredient with liquid carriers or finely divided solid carriers or both and then, if necessary, shaping the product into the desired formulation.

Pharmaceutical formulations of compounds provided herein (e.g., compounds of any one of Formulas I, IA, IA1, IA2, IB, IB1, IB2, IC, ID, ID′, IE, IF, or II in any available form (e.g., salt, ester, tautomer, prodrug, zwitterionic form, stereoisomer etc.)) may be provided as discrete units. For example, a formulation suitable for oral administration may be provided as capsules, cachets, and/or tablets containing a predetermined amount of the compound in any suitable form (e.g., the active ingredient); as a solution or suspension in a solvent (e.g., aqueous or non-aqueous solvent); as an emulsion (e.g., an oil-in-water liquid emulsion or water-in-oil liquid emulsion); or as a powder or granules. The active ingredient may additionally or alternatively be provided as a bolus, electuary, or paste.

Pharmaceutical preparations suitable for oral administration include tablets, push-fit capsules made of gelatin, as well as soft, sealed capsules made of gelatin and a plasticizer, such as glycerol or sorbitol. Tablets may be made by, for example, compression or molding, optionally with one or more accessory ingredients, such as one or more pharmaceutically acceptable excipients. Compressed tablets may be prepared by, for example, compressing in a suitable machine the active ingredient in a free-flowing form such as a powder or granules, optionally mixed with binders, inert diluents, or lubricating, surface active or dispersing agents. Molded tablets may be made by, for example, molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with, for example, one or more fillers such as lactose, one or more binders such as one or more starches, and/or one or more lubricants such as talc or magnesium stearate and, optionally, one or more stabilizers. In soft capsules, the active compound(s) may be dissolved or suspended in suitable liquids, such as fatty oils, liquid paraffin, or liquid polyethylene glycols. Stabilizers and other elements may also be added. Dragee cores are provided with suitable coatings. For this purpose, concentrated sugar solutions may be used, which may optionally contain a gum, gelling agent, polymer, solvent, or combination thereof. Dyestuffs or pigments may be added to the tablets or dragee coatings for identification or to characterize different combinations of active compound doses.

A pharmaceutical composition comprising a compound provided herein (e.g., a compound of any one of Formulas I, IA, IA1, IA2, IB, IB1, IB2, IC, ID, ID′, IE, IF, or II), or a form thereof (e.g., salt, ester, tautomer, prodrug, zwitterionic form, stereoisomer, etc.), may be formulated for parenteral administration by injection, e.g., by bolus injection or continuous infusion. Formulations for injection may be presented in unit dosage form, e.g., in ampoules, vials, or in multi-dose containers, with an added preservative. The compositions may take such forms as suspensions, solutions, or emulsions in oily or aqueous vehicles, and may contain formulating agents such as suspending, stabilizing, and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, prior (e.g., immediately prior) to use. Extemporaneous injection solutions and suspensions may be prepared from sterile powders, granules and tablets of the kind previously described.

A pharmaceutical composition comprising a compound provided herein (e.g., a compound of any one of Formulas I, IA, IA1, IA2, IB, IB1, IB2, IC, ID, ID′, IE, IF, or II), or a form thereof (e.g., salt, ester, tautomer, prodrug, zwitterionic form, stereoisomer etc.), may be formulated as a solution for injection, which solution may be an aqueous or non-aqueous (oily) sterile solution and may comprise one or more antioxidants, thickening agents, suspending agents, buffers, solutes, and/or bacteriostats. The addition of one or more such additives may render the formulation isotonic with the blood of the intended recipient (e.g., subject or patient). Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described elsewhere herein, the compounds provided herein (e.g., compounds of any one of Formulas I, IA, IA1, IA2, IB, IB1, IB2, IC, ID, ID′, IE, IF, or II) in any suitable form (e.g., salt, ester, tautomer, prodrug, zwitterionic form, stereoisomer, etc.), may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical composition comprising a compound provided herein (e.g., a compound of any one of Formulas I, IA, IA1, IA2, IB, IB1, IB2, IC, ID, ID′, IE, IF, or II) or a form thereof (e.g., salt, ester, tautomer, prodrug, zwitterionic form, stereoisomer, etc.) that is suitable for buccal or sublingual administration may take the form of tablets, lozenges, pastilles, or gels. Such compositions may comprise the active ingredient in a flavored basis such as sucrose and acacia or tragacanth. A pharmaceutical composition comprising a compound provided herein or a form thereof (e.g., salt, ester, tautomer, prodrug, zwitterionic form, stereoisomer, etc.) that is suitable for rectal administration may be formulated as a suppository or retention enema and may comprise a medium such as, for example, cocoa butter, polyethylene glycol, or other glycerides.

Certain compounds provided herein (e.g., a compound of any one of Formulas I, IA, IA1, IA2, IB, IB1, IB2, IC, ID, ID′, IE, IF, or II) or a form thereof (e.g., salt, ester, tautomer, prodrug, zwitterionic form, stereoisomer, etc.) may be formulated for non-systematic administration, such as topical administration. This includes the application of a compound disclosed herein, or a form thereof, externally to the epidermis or the buccal cavity and the instillation of such a compound, or a form thereof, into the ear, eye and nose, such that the compound, or a thereof, does not significantly enter the blood stream. In contrast, systemic administration refers to oral, intravenous, intraperitoneal, and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds (e.g., compounds of any one of Formulas I, IA, IA1, IA2, IB, IB1, IB2, IC, ID, ID′, IE, IF, or II) or forms thereof (e.g., salt, ester, tautomer, prodrug, zwitterionic form, stereoisomer, etc.) may be conveniently delivered from an insufflator, nebulizer pressurized packs, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compounds provided herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as described herein, or an appropriate fraction thereof, of the active ingredient (e.g., a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof).

It should be understood that in addition to the ingredients particularly described elsewhere herein, the formulations described herein may include other useful agents having regard to the type of formulation in question, for example those suitable for oral administration may include flavoring agents.

Compounds (e.g., compounds of any one of Formulas I, IA, IA1, IA2, IB, IB11, IB2, IC, ID, ID′, IE, IF, or II) or forms thereof (e.g., salt, ester, tautomer, prodrug, zwitterionic form, stereoisomer, etc.) may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

### Methods

The present disclosure also provides a method of modulating KRAS (e.g., KRAS having a G12D mutation) comprising contacting KRAS with a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof. For example, the present disclosure may provide a method of altering a cell phenotype, cell proliferation, KRAS activity, biochemical output produced by active or inactive KRAS, expression of KRAS, and/or binding of KRAS with a natural binding partner. Any such feature may be monitored and may be altered upon contacting KRAS with a compound provided herein, or a form thereof. A method of modulating KRAS (e.g., KRAS having a G12D mutation) may be a mode of treatment of a disease, disorder, or condition (e.g., a cancer), a biological assay, a cellular assay, a biochemical assay, etc.

The present disclosure also provides methods of treating a disease, disorder, or condition in a subject in need thereof using a compound provided herein, (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof. For example, the present disclosure provides a method comprising providing (e.g., administering) to a subject (e.g., patient) in need thereof an effective amount of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof. The present disclosure also provides methods of treating a disease, disorder, or condition in a subject in need thereof using a pharmaceutical composition comprising a compound provided herein, (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof. For example, the present disclosure provides a method comprising providing (e.g., administering) to a subject (e.g., patient) in need thereof a pharmaceutical composition comprising an effective amount of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof. In some embodiments, the subject is known to have (e.g., has previously been diagnosed with) a disease, disorder, or condition such as a cancer. The disease, disorder, or condition may be a KRAS-mediated disease, such as a cancer characterized by a G12D mutation in KRAS. In some embodiments, the compound administered to the subject in need thereof according to the methods described herein is a compound described in an embodiment, example, figure, or table herein, or a stereoisomer or pharmaceutically acceptable salt thereof.

The present disclosure also provides a compound as provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient, for use as a medicament, such as a medicament for the treatment of a disease, disorder, or condition (e.g., a cancer). The present disclosure also provides a compound as provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for the treatment of a disease, disorder, or condition (e.g., a cancer) in a subject in need thereof.

The present disclosure also provides the use of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient, for the treatment of a disease, disorder, or condition (e.g., a cancer) in a subject in need thereof.

The present disclosure also provides the use of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for treating a disease, disorder, or condition (e.g., a cancer) in a subject in need thereof.

The present disclosure also provides a method of inhibiting KRAS (e.g., KRAS having a G12D mutation) (e.g., in a subject in need thereof) comprising contacting KRAS with a compound as provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient.

The present disclosure also provides a compound as provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient, for use in inhibiting KRAS (e.g., KRAS having a G12D mutation) (e.g., in a subject in need thereof). The present disclosure also provides a compound as provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for inhibiting KRAS (e.g., KRAS having a G12D mutation) in a subject in need thereof.

The present disclosure also provides the use of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient, for inhibiting KRAS (e.g., KRAS having a G12D mutation) in a subject in need thereof.

The present disclosure also provides the use of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for inhibiting KRAS (e.g., KRAS having a G12D mutation) in a subject in need thereof.

The present disclosure also provides a method comprising administering a therapeutically effective amount of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof to a subject (e.g., patient), thereby ameliorating, reducing, eliminating, ceasing, or improving one or more symptoms of the subject, such as one or more symptoms of a disease, disorder, or condition (e.g., a cancer). In some embodiments, administering a therapeutically effective amount of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, slows or prevents growth of a tumor. In some embodiments, administering a therapeutically effective amount of a compound provided herein (e.g., a compound of any one of Formulas I, IA, IA1, IA2, IB, IB1, IB2, IC, ID, ID′, IE, IF, or II), or a salt, ester, tautomer, prodrug, zwitterionic form, or stereoisomer thereof, results in shrinkage of a tumor (e.g., tumor regression).

In some embodiments of any of the methods, uses, and medicaments provided herein, the disease, disorder, or condition is a cancer. In some embodiments, the cancer is pancreatic cancer (e.g., pancreatic ductal adenocarcinoma), lung cancer (e.g., non-small cell lung cancer), colorectal cancer (CRC), endometrial cancer, uterine carcinosarcoma, Ewing sarcoma, osteosarcoma, Rhabdomyosarcoma, adrenocortical carcinoma, neuroblastoma, Wilm tumor, retinoblastoma, skin cancer, breast cancer, prostate cancer, head and neck cancer, or ovarian cancer. In some embodiments, the cancer is pancreatic cancer (e.g., pancreatic ductal adenocarcinoma), lung cancer (e.g., non-small cell lung cancer adenocarcinoma), or colorectal cancer (CRC). In some embodiments, the cancer is pancreatic cancer (e.g., pancreatic ductal adenocarcinoma). In some embodiments, the cancer is lung cancer (e.g., non-small cell lung cancer adenocarcinoma). In some embodiments, the cancer is colorectal cancer (CRC). In some embodiments, the cancer is or comprises a solid tumor.

In some embodiments, the disease, disorder, or condition is related to KRAS, such as a disorder associated with a mutation of KRAS or dysregulation of KRAS. In some embodiments, the disease, disorder, or condition is related to the KRAS gene, such as a disease, disorder, or condition associated with a mutation of the KRAS gene or dysregulation of the KRAS gene. Mutation or dysregulation of KRAS or KRAS may include mutation or dysregulation of human K-Ras4a and/or human K-Ras4b. In some embodiments, the disease, disorder, or condition is related to the KRAS (e.g., human K-Ras4a or K-Ras4b) signaling pathway activity, such as a disease, disorder, or condition related to aberrant KRAS signaling pathway activity. In some embodiments, the disease, disorder, or condition is related to mutation or dysregulation of human K-Ras4b. In some embodiments, the disease, disorder, or condition is related to aberrant K-Ras4b signaling pathway activity. In some embodiments, the disease, disorder, or condition is related to mutation or dysregulation of human K-Ras4a. In some embodiments, the disease, disorder, or condition is related to aberrant K-Ras4a signaling pathway activity.

### Administration and Combination Therapy

The compounds provided herein (e.g., compounds of any one of Formulas I, IA, IA1, IA2, IB, IB1, IB2, IC, ID, ID′, IE, IF, or II) and forms thereof (e.g., salt, ester, tautomer, prodrug, zwitterionic form, stereoisomer, etc.), or compositions (e.g., pharmaceutical compositions) comprising the same, can be administered in various modes (e.g., orally, topically, or by injection). The amount of active ingredient (e.g., a compound provided herein in any suitable form) administered to a subject (e.g., patient) will be the responsibility of an attendant medical provider. The specific dose level for a given subject (e.g., patient) will depend on a variety of factors including, for example, the activity of the active ingredient administered; the physical attributes of the subject (e.g., age, weight, height, body mass index, general health, co-morbidities, sex, etc.); other characteristics of the subject (e.g., diet, level of exercise, national origin, ethnicity, etc.); time of administration; route of administration; rate of excretion; drug combination; the disease, disorder, or condition being treated; and the severity of the disease, disorder, or condition being treated.

In some embodiments, a compound provided herein (e.g., a compound of any one of Formulas I, IA, IA1, IA2, IB, IB1, IB2, IC, ID, ID′, IE, IF, or II), or a form thereof (e.g., salt, ester, tautomer, prodrug, zwitterionic form, stereoisomer, etc.) is administered in combination with an additional agent, such as an additional therapeutic agent. For example, if a subject experiences a side effect such as hypertension upon receiving a compound provided herein, or a form thereof, it may be appropriate to administer an additional agent that is effective in managing the side effect, such as an anti-hypertensive agent. In another example, the therapeutic effectiveness of a compound provided herein (e.g., a compound of any one of Formulas I, IA, IA1, IA2, IB, IB1, IB2, IC, ID, ID′, IE, IF, or II), or a form thereof, may be enhanced by administration of an adjuvant, which adjuvant may itself have only minimal therapeutic benefit, but in combination with another therapeutic agent may provide an enhanced overall therapeutic benefit to a subject. In a further example, the therapeutic benefit of a compound provided herein (e.g., a compound of any one of Formulas I, IA, IA1, IA2, IB, IB1, IB2, IC, ID, ID′, IE, IF, or II), or a form thereof, may be enhanced by administration of the compound, or its alternative form, and an additional agent (which may comprise an additional therapeutic regimen) that also provides a therapeutic benefit. For example, a compound provided herein (e.g., a compound of any one of Formulas I, IA, IA1, IA2, IB, IB1, IB2, IC, ID, ID′, IE, IF, or II), or a form thereof, may be administered in combination with an additional agent that may be effective in the treatment of a disease, disorder, or condition such as a cancer. Generally, the combination of a compound provided herein (e.g., a compound of any one of Formulas I, IA, IA1, IA2, IB, IB1, IB2, IC, ID, ID′, IE, IF, or II), or a form thereof, and one or more additional agents (e.g., therapeutic agents) may enhance the overall benefit experienced by the subject upon either component individually. In some embodiments, the effect may be additive. In some embodiments, the effect may be synergistic.

In some embodiments, a compound provided herein (e.g., a compound of any one of Formulas I, IA, IA1, IA2, IB, IB1, IB2, IC, ID, ID′, IE, IF, or II), or a form thereof (e.g., salt, ester, tautomer, prodrug, zwitterionic form, stereoisomer, etc.) is administered in combination with an anti-cancer agent (e.g., chemotherapeutic agent). An anti-cancer agent may be, for example, an alkylating agent, an antimitotic, a checkpoint inhibitor, an anti-metabolite, a plant alkaloid, a terpenoid, a cytotoxic agent, an antibiotic, a topoisomerase inhibitor, an aromatase inhibitor, an angiogenesis inhibitor, an anti-steroid, an anti-androgen, an mTOR inhibitor, monoclonal antibodies, or a tyrosine kinase inhibitor. An alkylating agent may be, for example, armustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, or cyclophosphamide (ENDOXAN). An anti-metabolite may be, for example, cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEY), methotrexate (RHEUMATREX), or raltitrexed. An antimitotic may be, for example, a taxane such as docetaxel (TAXITERE) or paclitaxel (ABRAXANE, TAXOL), or a vinca alkaloid such as vincristine (ONCOVIN), vinblastine, vindesine, or vinorelbine (NAVELBINE). A checkpoint inhibitor may be an anti-PD-1 or anti-PD-L1 antibody such as pembrolizumab (KEYTRUDA), nivolumab (OPDIVO), MEDI4736, or MPDL3280A; anti-CTLA-4 antibody ipilimumab (YERVOY); or an agent that targets LAG3 (lymphocyte activation gene 3 protein), KIR (killer cell immunoglobulin-like receptor), 4-1BB (tumor necrosis factor receptor superfamily member 9), TIM3 (T-cell immunoglobulin and mucin-domain containing-3), or 0X40 (tumor necrosis factor receptor superfamily member 4). A topoisomerase inhibitor may be, for example, camptothecin (CTP), irinotecan (CAMPTOSAR), topotecan (HYCAMTIN), teniposide (VUMON), or etoposide (EPOSIN). A cytotoxic antibiotic may be, for example, actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOYANTRONE), or plicamycin. An aromatase inhibitor may be, for example, aminoglutethimide, anastrozole (ARIMIDEX), letrozole (FEMARA), vorozole (RIYIZOR), or exemestane (AROMASIN). An angiogenesis inhibitor may be, for example, genistein, sunitinib (SUTENT), or bevacizumab (AYASTIN). An anti-steroid or anti-androgen may be, for example, aminoglutethimide (CYTADREN), bicalutamide (CASODEX), cyproterone, flutamide (EULEXIN), or nilutamide (NILANDRON). A tyrosine kinase inhibitor may be, for example, imatinib (GLEEVEC), erlotinib (TARCEVA), afatinib (GILOTRIF), lapatinib (TYKERB), sorafenib (NEXAVAR), or axitinib (INLYTA). An mTOR inhibitor may be, for example, everolimus, temsirolimus (TORISEL), or sirolimus. Monoclonal antibody may be, for example, trastuzumab (HERCEPTIN) or rituximab (RITUXAN). Additional examples of agents that may be useful in combination with a compound provided herein, or an alternative form thereof, include, but are not limited to, amsacrine; Bacillus Calmette-Guerin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,r′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

Two or more therapeutic agents, one of which is a compound provided herein (e.g., a compound of any one of Formulas I, IA, IA1, IA2, IB, IB1, IB2, IC, ID, ID′, IE, IF, or II) or a form thereof, may be administered in any order or may be administered simultaneously. If administered simultaneously, the multiple therapeutic agents may be provided in a single, unified form, or in multiple forms (such as, for example, as a single pill or as two separate pills). One of the therapeutic agents may be given in multiple doses, or both may be given as multiple doses. If not administered simultaneously, the timing between the multiple doses may be any duration of time ranging from a few minutes to four weeks.

Accordingly, in another aspect, the present disclosure provides a method for treating a disease, disorder, or condition (e.g., a cancer) in a subject (e.g., a human or animal subject) in need of such treatment comprising administering to the subject an amount of a compound provided herein (e.g., a compound of any one of Formulas I, IA, IA1, IA2, IB, IB1, IB2, IC, ID, ID′, IE, IF, or II), or a form thereof (e.g., salt, ester, tautomer, prodrug, zwitterionic form, stereoisomer, etc.), in combination with at least one additional agent for the treatment of the disease, disorder, or condition. In a related aspect, the present disclosure provides a composition (e.g., pharmaceutical composition) comprising a compound provided herein (e.g., a compound of any one of Formulas I, IA, IA1, IA2, IB, IB1, IB2, IC, ID, ID′, IE, IF, or II), or a form thereof (e.g., salt, ester, tautomer, prodrug, zwitterionic form, stereoisomer, etc.), and at least one additional agent for use in the treatment of a disease, disorder, or condition (e.g., a cancer).

In some embodiments, a method provided herein is used to treat a disease, disorder, or condition (e.g., a cancer) comprising administering to a subject in need thereof a therapeutically effective amount of a compound of any one of Formulas I, IA, IA1, IA2, IB, IB1, IB2, IC, ID, ID′, IE, IF, or II or a pharmaceutically acceptable salt thereof, wherein the disease, disorder, or condition is a cancer that has developed a resistance to one or more chemotherapeutic drugs and/or ionizing radiation. In some embodiments, a method provided herein is used to treat a disease, disorder, or condition (e.g., a cancer) comprising administering to a subject in need thereof a therapeutically effective amount of a compound of any one of Formulas I, IA, IA1, IA2, IB, IB1, IB2, IC, ID, ID′, IE, IF, or II or a pharmaceutically acceptable salt thereof, in combination with an additional agent, wherein the disease, disorder, or condition is a cancer that has developed a resistance to one or more chemotherapeutic drugs and/or ionizing radiation.

The compounds, compositions, and methods disclosed herein are useful for the treatment of a disease, disorder, or condition, such as a cancer. In certain embodiments, the disease is one of dysregulated cellular proliferation, including cancer. The cancer may be hormone-dependent or hormone-resistant, such as in the case of breast cancers. In certain embodiments, the cancer is or comprises a solid tumor. In other embodiments, the cancer is a lymphoma or leukemia. In certain embodiments, the cancer is a drug resistant phenotype of a cancer disclosed herein or otherwise known. Tumor invasion, tumor growth, tumor metastasis, and angiogenesis may also be treated using the compositions and methods disclosed herein. In some embodiments, the compounds, compositions, and methods provided herein are also useful in the treatment of precancerous neoplasias.

Cancers that may be treated by the methods disclosed herein include, but are not limited to, pancreatic cancer, colon cancer, rectal cancer, colorectal cancer, breast cancer, ovarian cancer, endometrial cancer, lung cancer, and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic lymphomas. Additional types of cancers which may be treated using the compounds and methods provided herein include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor. Additional diseases and disorders that may be treated by the methods disclosed herein include, but are not limited to, diseases or disorders related to KRAS, such as diseases or disorders associated with a mutation of KRAS or dysregulation of KRAS, and diseases or disorders related to the KRAS gene, such as diseases or disorders associated with a mutation of the KRAS gene or dysregulation of the KRAS gene.

In some embodiments, the compounds, compositions, and methods provided herein are useful in the prevention and/or reduction of tumor invasion, growth, and/or metastasis.

The compounds, compositions, and methods provided herein may be useful in the treatment of humans as well as in the veterinary treatment of non-human animals including companion animals, exotic animals, and farm animals (e.g., as described herein), including mammals, rodents, and the like. For example, the compounds, compositions, and methods provided herein may be useful in the treatment of horses, dogs, or cats.

### ENUMERATED EMBODIMENTS

Embodiment I-1. A compound represented by Formula I:

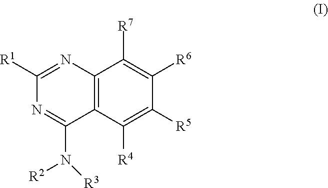

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-2. The compound of embodiment I-1, wherein R¹ is —OR⁸.

Embodiment I-3. The compound of embodiment I-2, wherein R⁸ is a heterocycle.

Embodiment I-4. The compound of embodiment I-3, wherein R⁸ is an alkylheterocycle.

Embodiment I-5. The compound of embodiment I-3 or I-4, wherein R⁸ comprises a heterocycle comprising at least one nitrogen atom.

Embodiment I-6. The compound of any one of embodiments I-3 to I-5, wherein the heterocycle comprises one or R¹⁶ substituents.

Embodiment I-7. The compound of embodiment I-6, wherein at least one R¹⁶ is —OR¹², where R¹² is independently selected from C₁₋₆ alkyl, C₂₋₆ alkenyl, and H.

Embodiment I-8. The compound of embodiment I-7, wherein at least one R¹⁶ is —OCH₃.

Embodiment I-9. The compound of embodiment I-1, wherein R¹ is selected from:

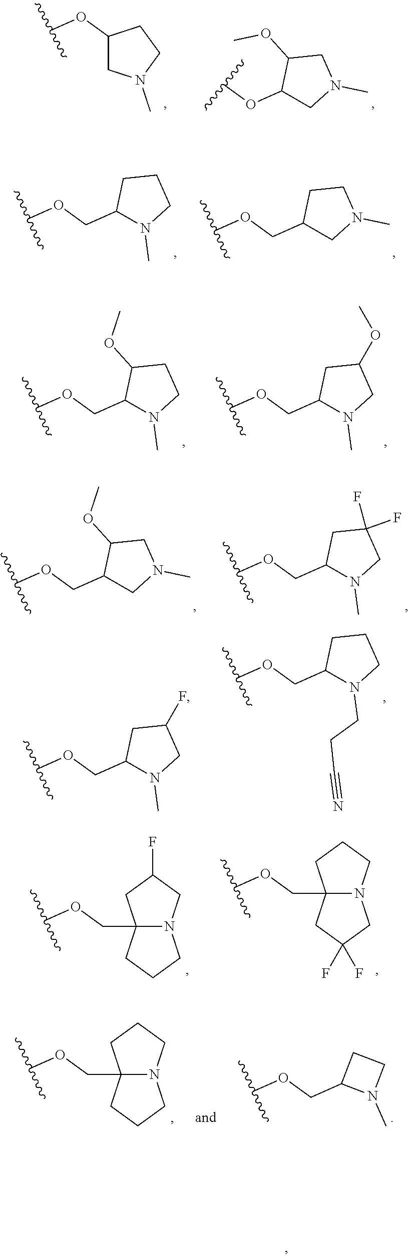

Embodiment I-10. The compound of embodiment I-1, wherein R¹ is selected from:

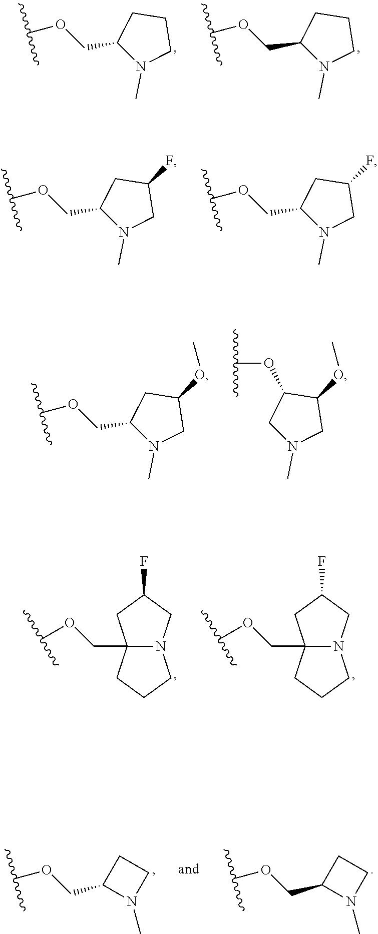

Embodiment I-11. The compound of embodiment I-1, wherein R¹ is a 4-6 membered heterocycle comprising a nitrogen atom, which heterocycle is unsubstituted or substituted with one or more R¹⁶.

Embodiment I-12. The compound of embodiment I-11, wherein R¹ is selected from:

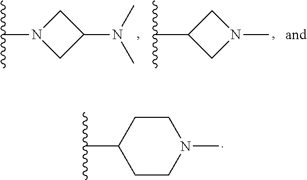

Embodiment I-13. The compound of embodiment I-1, wherein R¹ is H.

Embodiment I-14. The compound of any one of embodiments I-1 to I-13, wherein R² is H.

Embodiment I-15. The compound of any one of embodiments I-1 to I-13, wherein R² is C₁₋₆ alkyl.

Embodiment I-16. The compound of any one of embodiments I-1 to I-15, wherein R³ is C₁₋₆ alkyl, which C₁₋₆alkyl is substituted with one or more R⁹.

Embodiment I-17. The compound of embodiment I-16, wherein R³ is C₁₋₆ alkyl substituted with —N(R¹⁷)₂, where each R¹⁷ is independently selected from C₁₋₆ alkyl and H.

Embodiment I-18. The compound of embodiment I-17, wherein R³ is C₁₋₆ alkyl substituted with —NH₂.

Embodiment I-19. The compound of any one of embodiments I-1 to I-15, wherein R³ is selected from:

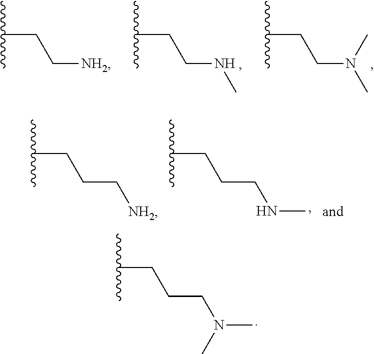

Embodiment I-20. The compound of any one of embodiments I-1 to I-15, wherein R³ is a carbocycle, which carbocycle is unsubstituted or substituted with one or more R¹⁰.

Embodiment I-21. The compound of embodiment I-20, wherein R³ is a carbocycle that is substituted with —N(R¹⁹)₂, where each R¹⁹ is independently selected from unsubstituted or substituted C₁₋₆ alkyl and H.

Embodiment I-22. The compound of embodiment I-21, wherein R³ is a carbocycle that is substituted with —NH₂.

Embodiment I-23. The compound of any one of embodiments I-1 to I-15, wherein R³ is selected from C₁₋₆alkyl-N(R¹⁷)C(O)C₁₋₆alkylN(R¹⁷)₂.

Embodiment I-24. The compound of any one of embodiments I-1 to I-15, wherein R³ is a heterocycle, which heterocycle is unsubstituted or substituted with one or more R¹⁰.

Embodiment I-25. The compound of embodiment I-24, wherein the heterocycle comprises one or more nitrogen atoms.

Embodiment I-26. The compound of embodiment I-24 or I-25, wherein R³ is a heterocycle that is substituted with —N(R¹⁹)₂, where each R¹⁹ is independently selected from unsubstituted or substituted C₁₋₆ alkyl and H.

Embodiment I-27. The compound of embodiment I-26, wherein R³ is a heterocycle that is substituted with —NH₂.

Embodiment I-28. The compound of any one of embodiments I-1 to I-15, wherein R³ is selected from:

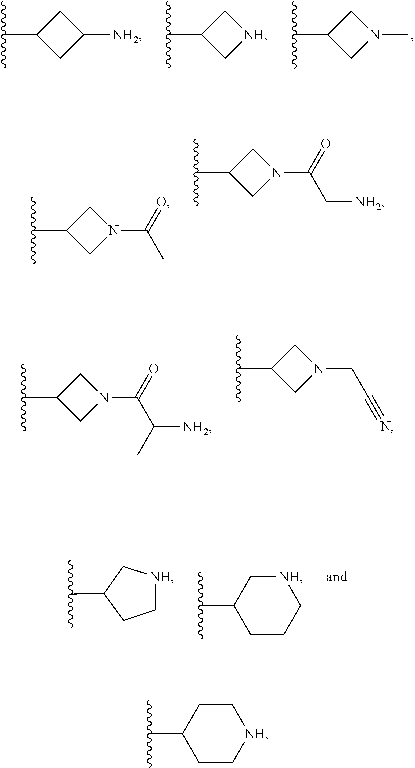

any of which is unsubstituted or substituted with one or more R¹⁰.

Embodiment I-29. The compound of any one of embodiments I-15 to I-28, wherein R² or R³ comprises an amino moiety.

Embodiment I-30. The compound of any one of embodiments I-1 to I-13, wherein R² and R³, together with the atom (e.g., nitrogen atom) to which they are attached, form a heterocycle that is unsubstituted or substituted with one or more R¹¹.

Embodiment I-31. The compound of embodiment I-30, wherein R² and R³, together with the atom to which they are attached, form a heterocycle having the structure:

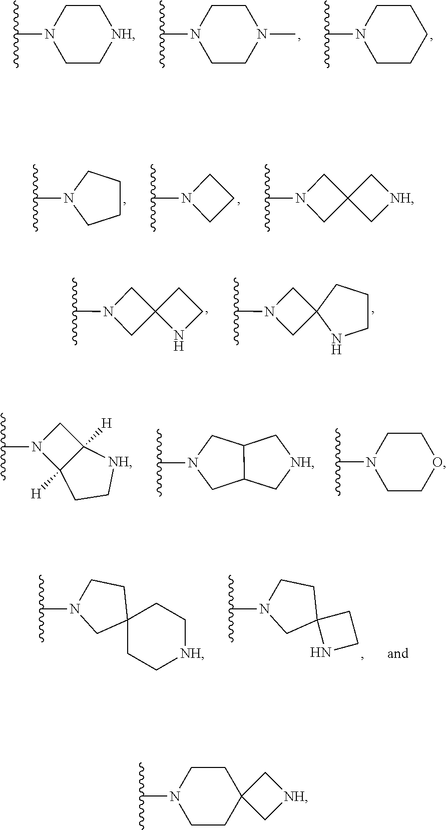

any of which is unsubstituted or substituted with one or more R¹¹.

Embodiment I-32. The compound of embodiment I-30, wherein R² and R³, together with the atom to which they are attached, form a heterocycle having the structure:

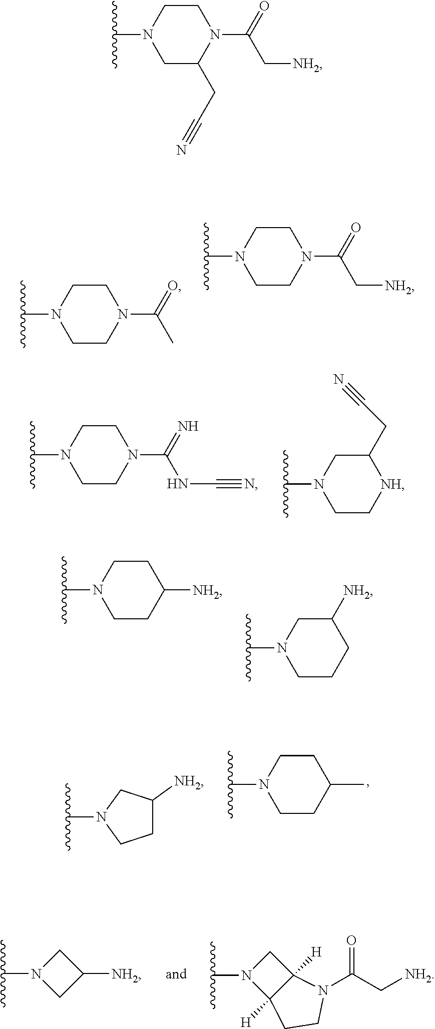

Embodiment I-33. The compound of any one of embodiments I-30 to I-32, wherein R² and R³, together with the nitrogen atom to which they are attached, form a heterocycle that (i) comprises an additional nitrogen atom or (ii) is substituted with a group comprising an amino moiety.

Embodiment I-34. The compound of any one of embodiments I-1 to I-33, wherein R⁴ is hydrogen.

Embodiment I-35. The compound of any one of embodiments I-1 to I-33, wherein R⁴ is a halogen.

Embodiment I-36. The compound of any one of embodiments I-1 to I-35, wherein R⁵ is a halogen.

Embodiment I-37. The compound of any one of embodiments I-1 to I-36, wherein R⁷ is a halogen.

Embodiment I-38. The compound of any one of embodiments I-1 to I-37, wherein R⁶ is selected from:

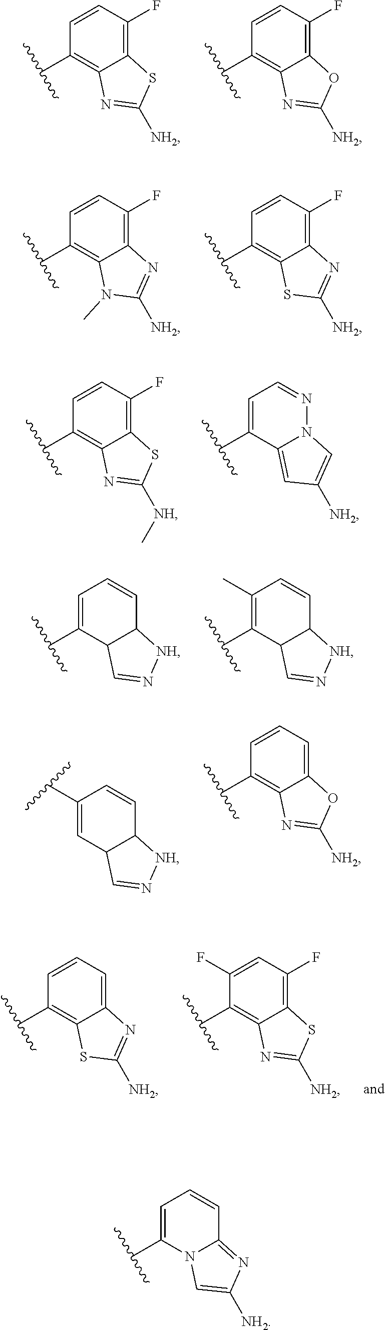

Embodiment I-39. The compound of embodiment I-38, wherein R⁶ is selected from:

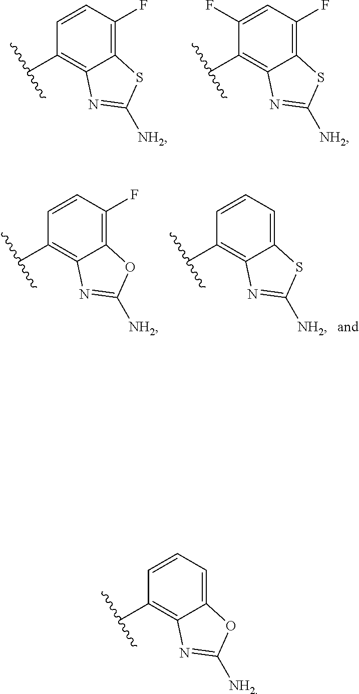 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; "and" ; *c1cccc2oc(N)nc12]

Embodiment I-40. The compound of embodiment I-39, wherein R⁶ is:

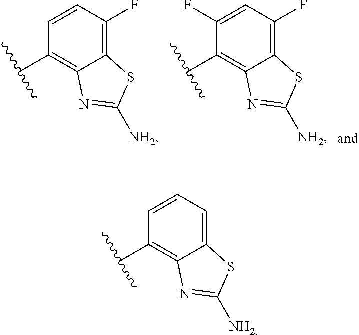 [SMILES: *c1ccc(F)c2sc(N)nc12 ; *c1c(F)cc(F)c2sc(N)nc12 ; "and" ; *c1cccc2sc(N)nc12]

Embodiment I-41. A compound represented by Formula IA:

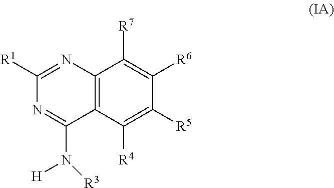

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
R² is H;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-42. The compound of embodiment I-41, wherein R³ is selected from a carbocycle and a heterocycle, wherein the carbocycle or heterocycle is unsubstituted or substituted with one or more R¹⁰.

Embodiment I-43. The compound of embodiment I-42, wherein R³ is a 3-6 membered carbocycle or heterocycle that is unsubstituted or substituted with one or more R¹⁰.

Embodiment I-44. The compound of embodiment I-43, wherein R³ is selected from:

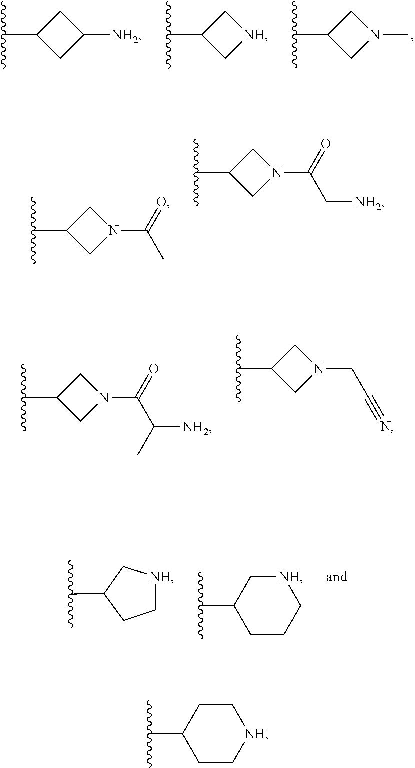

any of which is unsubstituted or substituted with one or more R¹⁰.

Embodiment I-45. The compound of embodiment I-41, wherein R³ is selected from C₁₋₆ alkyl that is substituted with one or more R⁹.

Embodiment I-46. The compound of embodiment I-45, wherein R³ is selected from C₁₋₆alkyl-N(R¹⁷)₂.

Embodiment I-47. The compound of embodiment I-45 or I-46, wherein R³ is selected from:

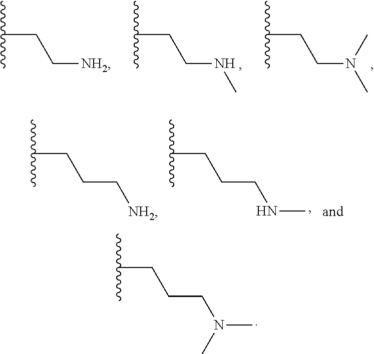

Embodiment I-48. The compound of embodiment I-45, wherein R³ is selected from C₁₋₆alkyl-N(R¹⁷)C(O)C₁₋₆alkylN(R¹⁷)₂.

Embodiment I-49. The compound of any one of embodiments I-41 to I-48, wherein R³ comprises an amino moiety.

Embodiment I-50. The compound of any one of embodiments I-41 to I-49, wherein R¹ is H.

Embodiment I-51. The compound of any one of embodiments I-41 to I-50, wherein R¹ is —OR⁸.

Embodiment I-52. The compound of embodiment I-51, wherein R⁸ comprises a 3-6 membered heterocycle that is unsubstituted or substituted with one or more R¹⁴.

Embodiment I-53. The compound of embodiment I-51 or I-52, wherein R¹ is selected from:

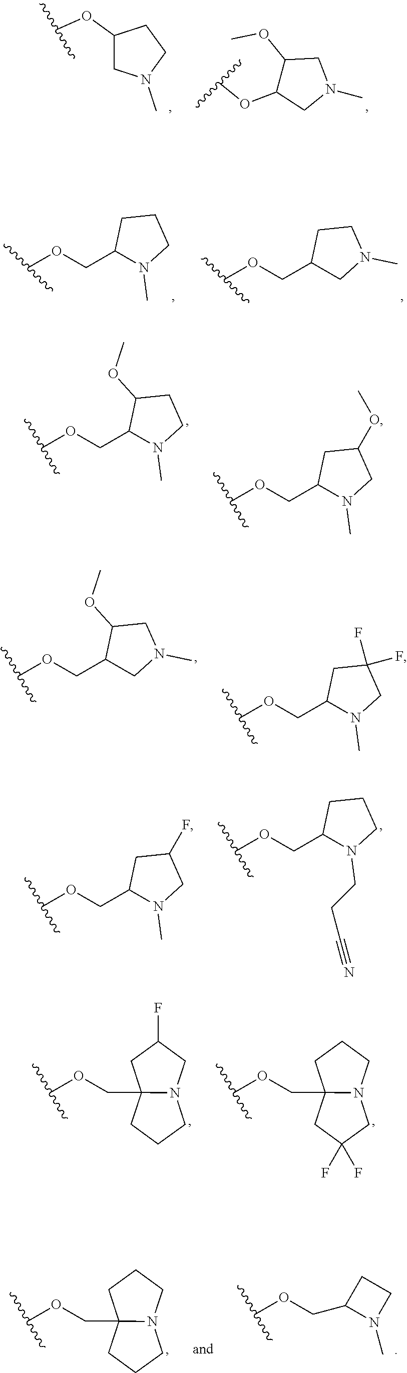

Embodiment I-54. The compound of embodiment I-51 or I-52, wherein R¹ is selected from:

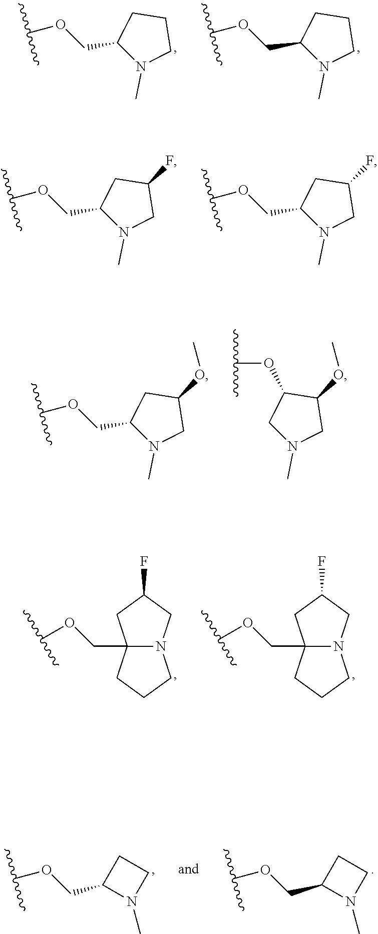

Embodiment I-55. The compound of any one of embodiments I-41 to I-54, wherein R¹ is a 4-6 membered heterocycle comprising a nitrogen atom that is unsubstituted or substituted with one or more R¹⁶.

Embodiment I-56. The compound of embodiment I-55, wherein R¹ is selected from:

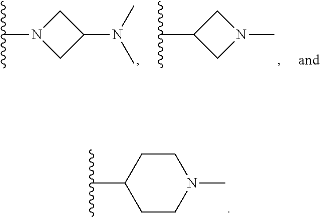

Embodiment I-57. The compound of any one of embodiments I-41 to I-56, wherein R⁶ is selected from:

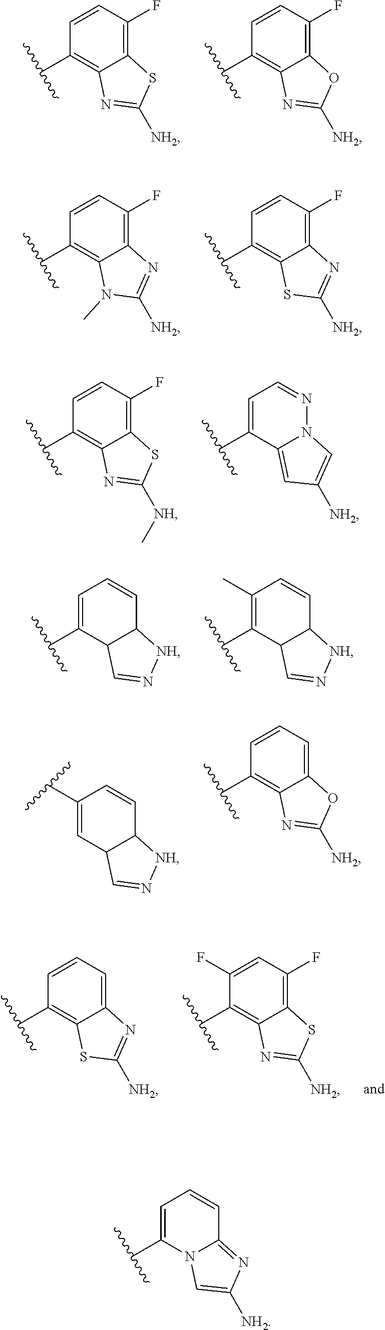

Embodiment I-58. The compound of embodiment I-57, wherein R⁶ is selected from:

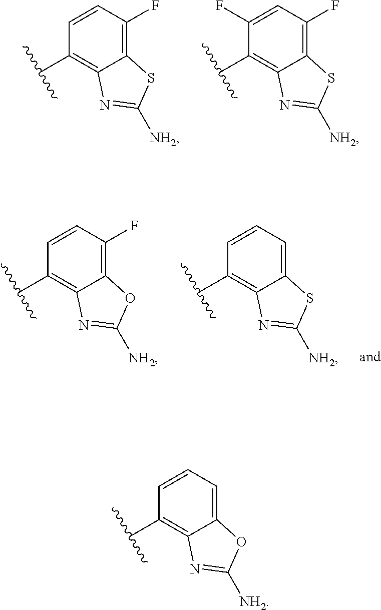 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; *c1cccc2oc(N)nc12]

Embodiment I-59. The compound of embodiment I-58, wherein R⁶ is:

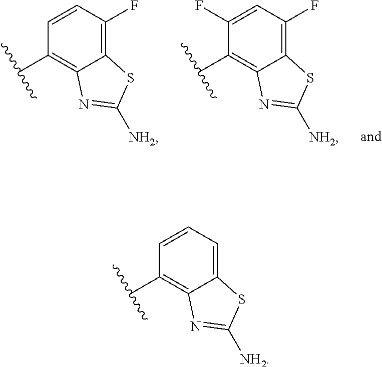 [SMILES: *c1ccc(F)c2sc(N)nc12 ; *c1c(F)cc(F)c2sc(N)nc12 ; *c1cccc2sc(N)nc12]

Embodiment I-60. The compound of any one of embodiments I-41 to I-59, wherein R⁴ is H.

Embodiment I-61. The compound of any one of embodiments I-41 to I-59, wherein R⁴ is a halogen.

Embodiment I-62. The compound of any one of embodiments I-41 to I-61, wherein R⁵ is a halogen.

Embodiment I-63. The compound of any one of embodiments I-41 to I-62, wherein R⁷ is a halogen.

Embodiment I-64. A compound according to Formula IA1:

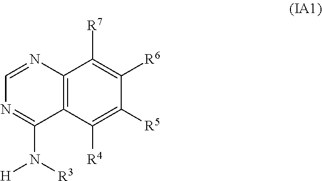

or a salt (e.g., pharmaceutically acceptable salt), ester, tautomer, prodrug, zwitterionic form, or stereoisomer thereof, wherein:
R¹ is H;
R² is H;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-65. The compound of embodiment I-64, wherein R³ is selected from a carbocycle, and a heterocycle, wherein the carbocycle or heterocycle is unsubstituted or substituted with one or more R¹⁰.

Embodiment I-66. The compound of embodiment I-65, wherein R³ is selected from:

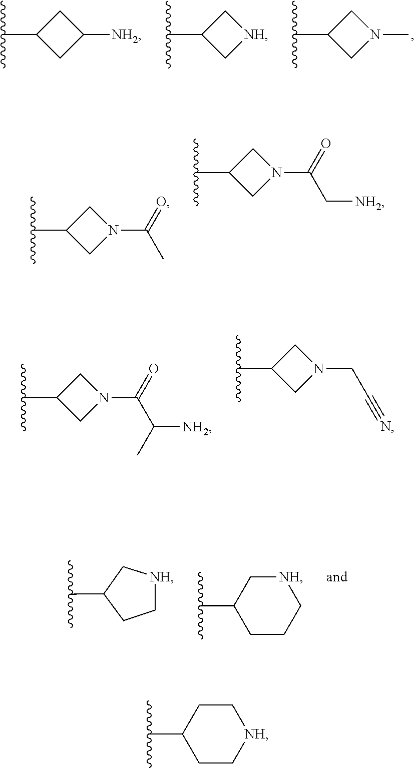

any of which is unsubstituted or substituted with one or more R¹⁰.

Embodiment I-67. The compound of embodiment I-64, wherein R³ is selected from C₁₋₆ alkyl that is substituted with one or more R⁹.

Embodiment I-68. The compound of embodiment I-67, wherein R³ is selected from:

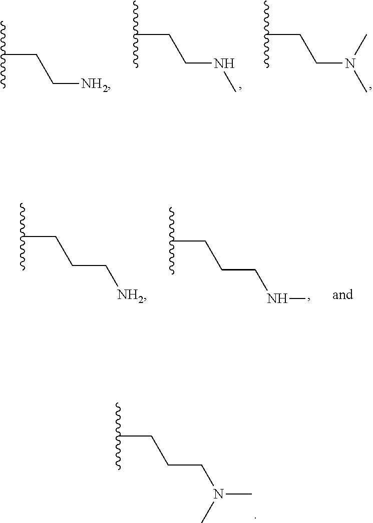

Embodiment I-69. The compound of embodiment I-64, wherein R³ is selected from C₁₋₆alkyl-N(R⁷)C(O)C₁₋₆alkylN(R⁷)₂.

Embodiment I-70. The compound of any one of embodiments I-64 to I-68, wherein R³ comprises an amino moiety.

Embodiment I-71. The compound of any one of embodiments I-62 to I-70, wherein R⁶ is selected from:

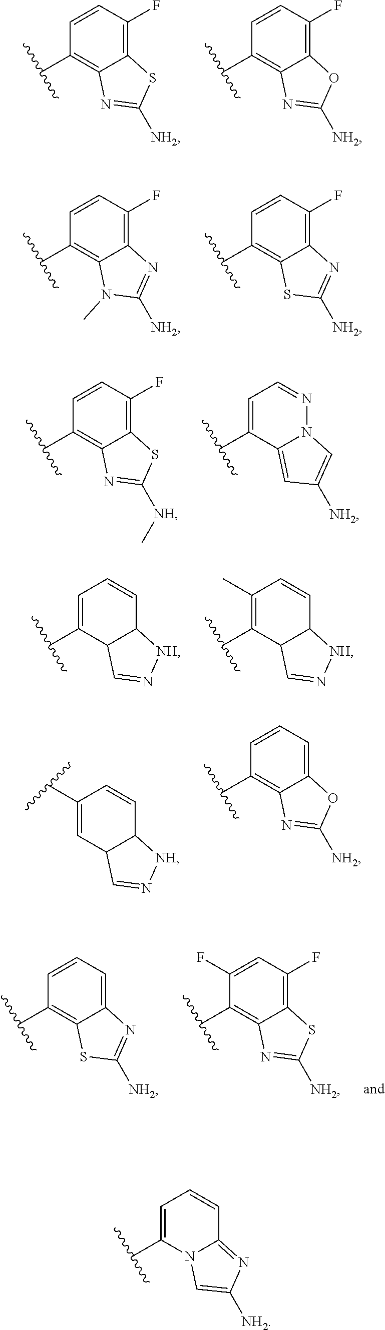

Embodiment I-72. The compound of embodiment I-71, wherein R⁶ is selected from:

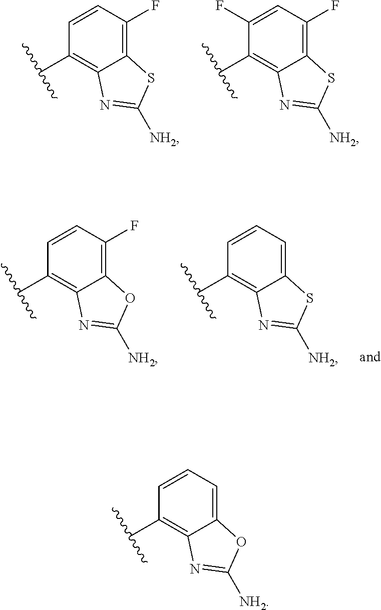 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; *c1cccc2oc(N)nc12]

Embodiment I-73. The compound of embodiment I-72, wherein R⁶ is:

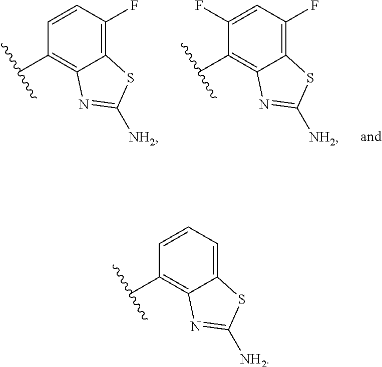 [SMILES: *c1ccc(F)c2sc(N)nc12 ; *c1c(F)cc(F)c2sc(N)nc12 ; *c1cccc2sc(N)nc12]

Embodiment I-74. The compound of any one of embodiments I-64 to I-73, wherein R⁴ is H.

Embodiment I-75. The compound of any one of embodiments I-64 to I-73, wherein R⁴ is a halogen.

Embodiment I-76. The compound of any one of embodiments I-64 to I-75, wherein R⁵ is a halogen.

Embodiment I-77. The compound of any one of embodiments I-64 to I-76, wherein R⁷ is a halogen.

Embodiment I-78. A compound according to Formula IA2:

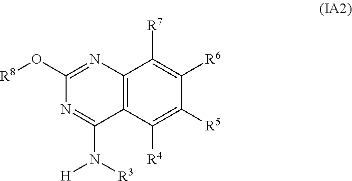

or a salt (e.g., pharmaceutically acceptable salt), ester, tautomer, prodrug, zwitterionic form, or stereoisomer thereof, wherein:
R¹ is —OR⁸;
R² is H;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-79. The compound of embodiment I-78, wherein R⁸ comprises a 3-6 membered heterocycle that is unsubstituted or substituted with one or more R¹⁴.

Embodiment I-80. The compound of embodiment I-78, wherein R¹ is selected from:

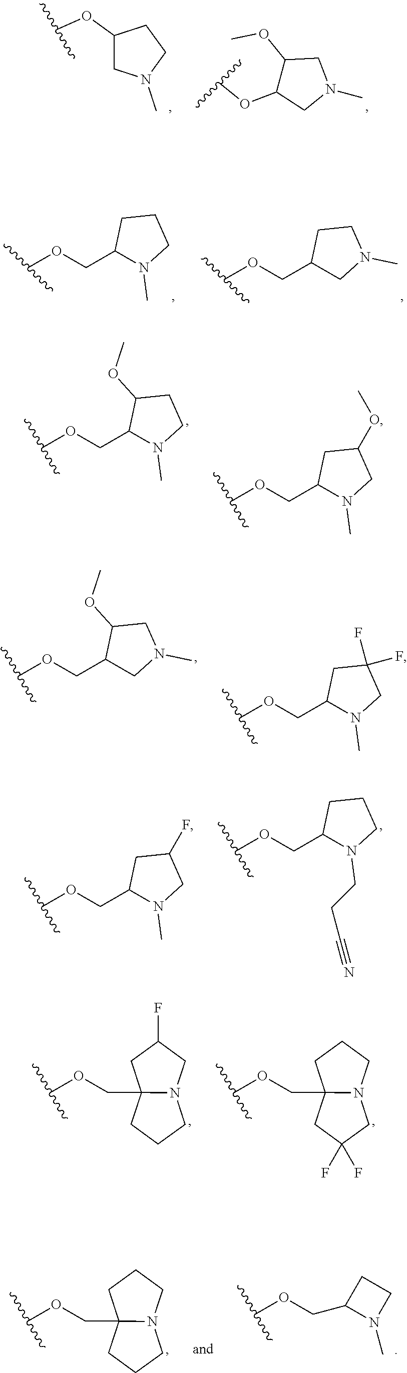

Embodiment I-81. The compound of embodiment I-78, wherein R¹ is selected from:

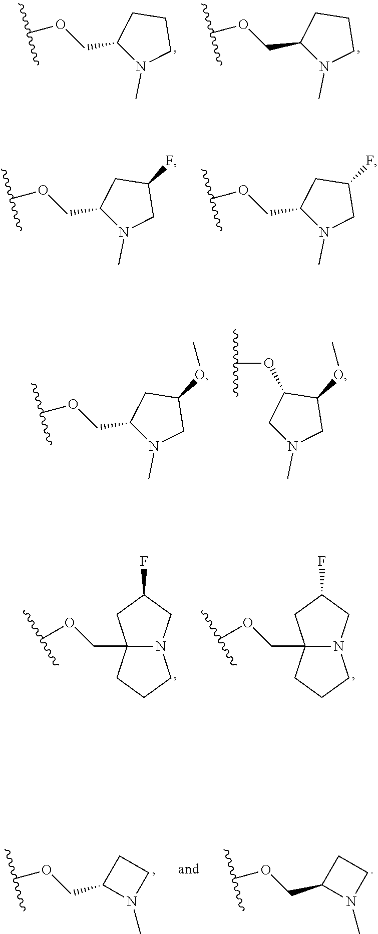

Embodiment I-82. The compound of any one of embodiments I-78 to I-81, wherein R³ is selected from a carbocycle, and a heterocycle, wherein the carbocycle or heterocycle is unsubstituted or substituted with one or more R¹⁰.

Embodiment I-83. The compound of embodiment I-82, wherein R³ is selected from:

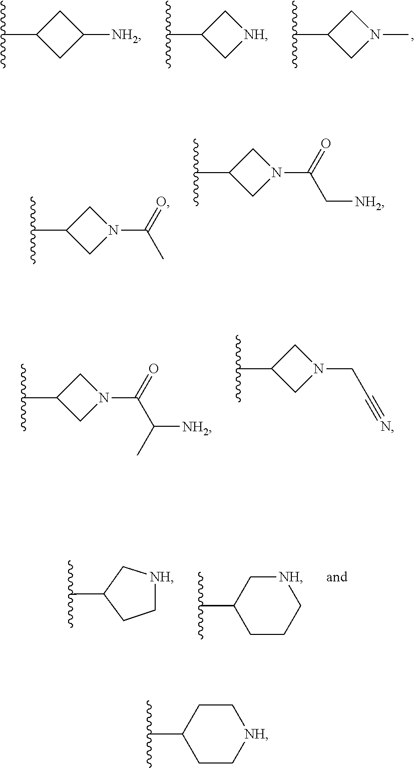

any of which is unsubstituted or substituted with one or more R¹⁰.

Embodiment I-84. The compound of any one of embodiments I-78 to I-83, wherein R³ is selected from C₁₋₆ alkyl that is substituted with one or more R⁹.

Embodiment I-85. The compound of embodiment I-84, wherein R³ is selected from:

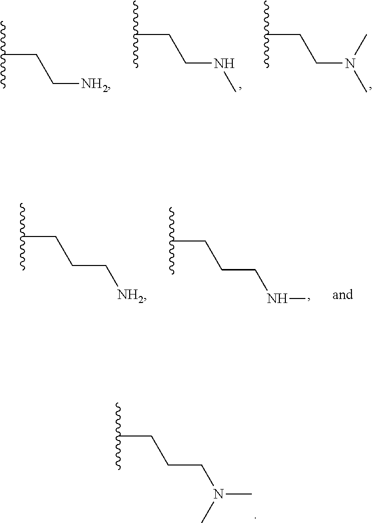

Embodiment I-86. The compound of any one of embodiments I-78 to I-85, wherein R³ is selected from C₁₋₆alkyl-N(R¹⁷)C(O)C₁₋₆alkylN(R¹⁷)₂.

Embodiment I-87. The compound of any one of embodiments I-78 to I-86, wherein R³ comprises an amino moiety.

Embodiment I-88. The compound of any one of embodiments I-78 to I-87, wherein R⁶ is selected from:

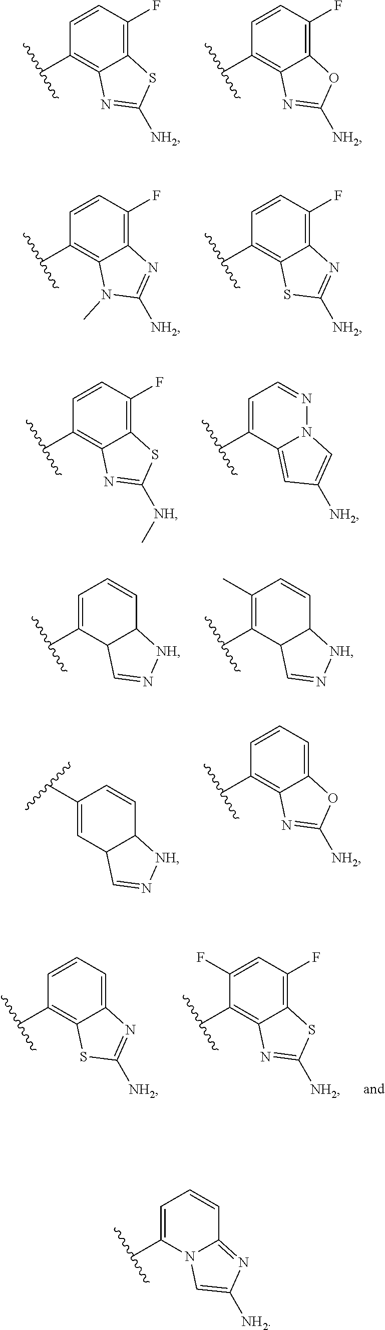

Embodiment I-89. The compound of embodiment I-88, wherein R⁶ is selected from:

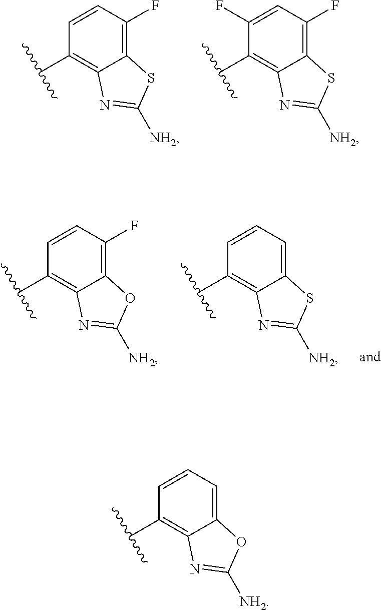 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; *c1cccc2oc(N)nc12]

Embodiment I-90. The compound of embodiment I-89, wherein R⁶ is:

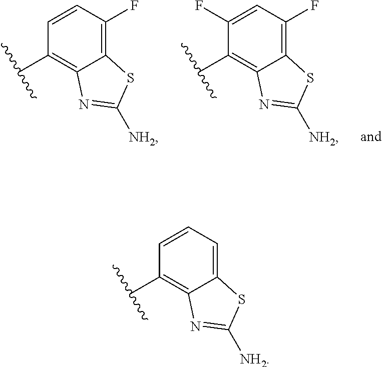 [SMILES: *c1ccc(F)c2sc(N)nc12 ; *c1c(F)cc(F)c2sc(N)nc12 ; *c1cccc2sc(N)nc12]

Embodiment I-91. The compound of any one of embodiments I-78 to I-90, wherein R⁴ is H.

Embodiment I-92. The compound of any one of embodiments I-78 to I-90, wherein R⁴ is a halogen.

Embodiment I-93. The compound of any one of embodiments I-78 to I-92, wherein R⁵ is a halogen.

Embodiment I-94. The compound of any one of embodiments I-78 to I-93, wherein R⁷ is a halogen.

Embodiment I-95. A compound according to Formula IB:

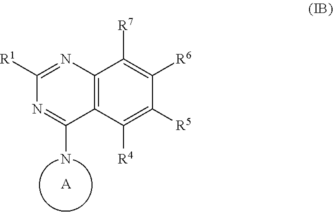

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
Ring A is a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-96. The compound of embodiment I-95, wherein Ring A is a 4-6 membered heterocycle that is unsubstituted or substituted with one or more R¹¹.

Embodiment I-97. The compound of embodiment I-95, wherein Ring A has the structure:

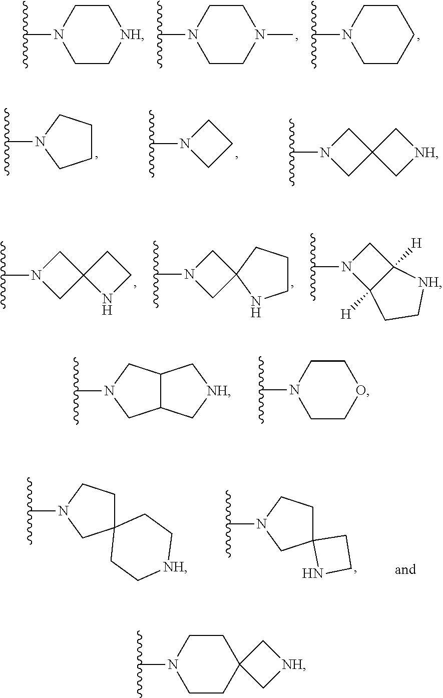

which ring is unsubstituted or substituted with one or more R¹¹.

Embodiment I-98. The compound of embodiment I-95, wherein Ring A has the structure:

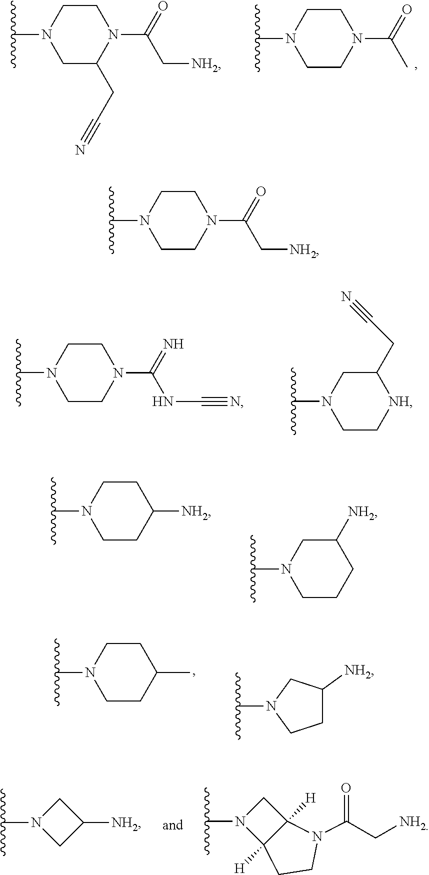

Embodiment I-99. The compound of any one of embodiments I-95 to I-98, wherein Ring A includes at least two nitrogen atoms and/or is substituted with a group including an amino moiety.

Embodiment I-100. The compound of any one of embodiments I-95 to I-99, wherein R¹ is H.

Embodiment I-101. The compound of any one of embodiments 1-95 to 1-99, wherein R¹ is —OR⁸.

Embodiment I-102. The compound of embodiment I-101, wherein R⁸ comprises a 3-6 membered heterocycle that is unsubstituted or substituted with one or more R¹⁴.

Embodiment I-103. The compound of embodiment I-101, wherein R¹ is selected from:

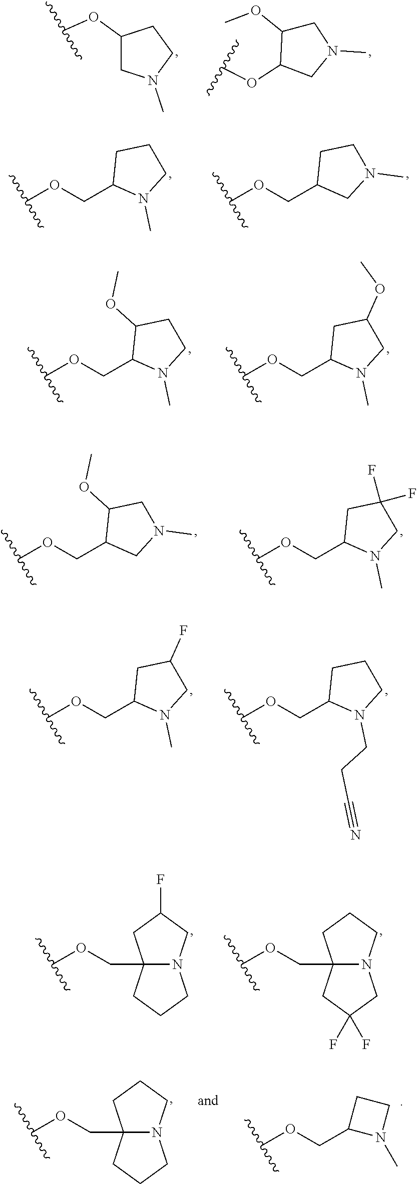

Embodiment I-104. The compound of embodiment I-101, wherein R¹ is selected from:

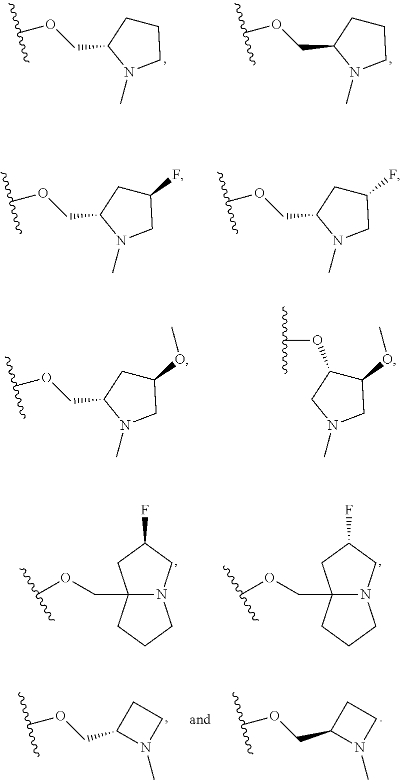

Embodiment I-105. The compound of any one of embodiments I-95 to I-99, wherein R¹ is a 4-6 membered heterocycle comprising a nitrogen atom that is unsubstituted or substituted with one or more R¹⁶.

Embodiment I-106. The compound of embodiment I-105, wherein R¹ is selected from:

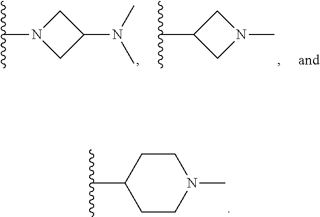

Embodiment I-107. The compound of any one of embodiments I-95 to I-106, wherein R⁶ is selected from:

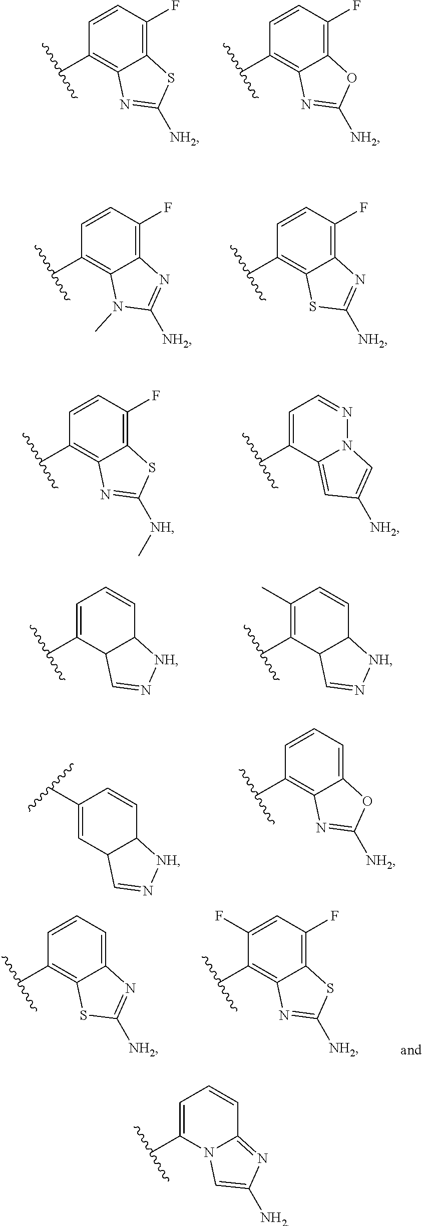

Embodiment I-108. The compound of embodiment I-107, wherein R⁶ is selected from:

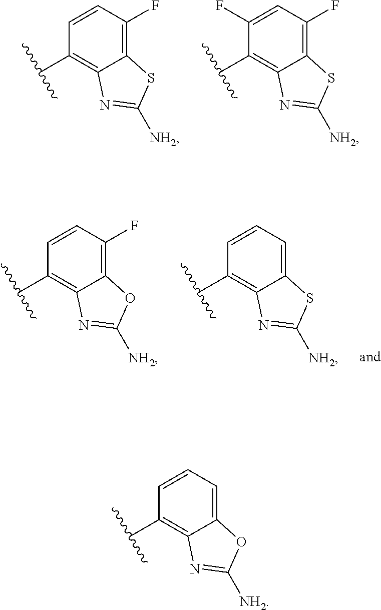 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; *c1cccc2oc(N)nc12]

Embodiment I-109. The compound of embodiment I-108, wherein R⁶ is:

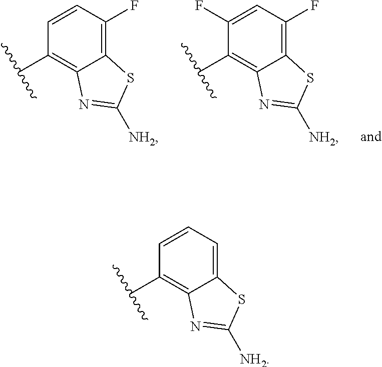 [SMILES: *c1ccc(F)c2sc(N)nc12 ; *c1c(F)cc(F)c2sc(N)nc12 ; *c1cccc2sc(N)nc12]

Embodiment I-110. The compound of any one of embodiments I-95 to I-109, wherein R⁴ is H.

Embodiment I-111. The compound of any one of embodiments I-95 to I-109, wherein R⁴ is a halogen.

Embodiment I-112. The compound of any one of embodiments I-95 to I-111, wherein R⁵ is a halogen.

Embodiment I-113. The compound of any one of embodiments I-95 to I-112, wherein R⁷ is a halogen.

Embodiment I-114. A compound according to Formula IB1:

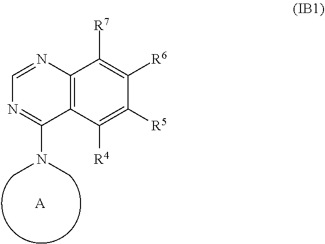

or a salt (e.g., pharmaceutically acceptable salt), ester, tautomer, prodrug, zwitterionic form, or stereoisomer thereof, wherein:
Ring A is a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R⁵;
R⁷ is selected from halogen, —OR¹², —CN, and H;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-115. The compound of embodiment I-114, wherein Ring A is a 4-6 membered heterocycle that is unsubstituted or substituted with one or more R¹¹.

Embodiment I-116. The compound of embodiment I-114, wherein Ring A has the structure:

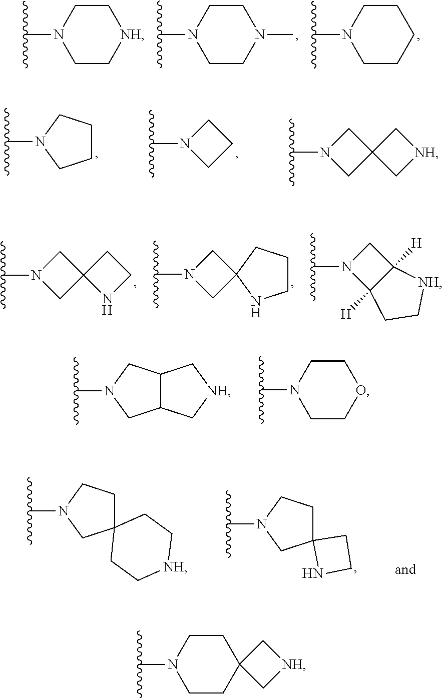

which ring is unsubstituted or substituted with one or more R¹¹.

Embodiment I-117. The compound of embodiment I-114, wherein Ring A has the structure:

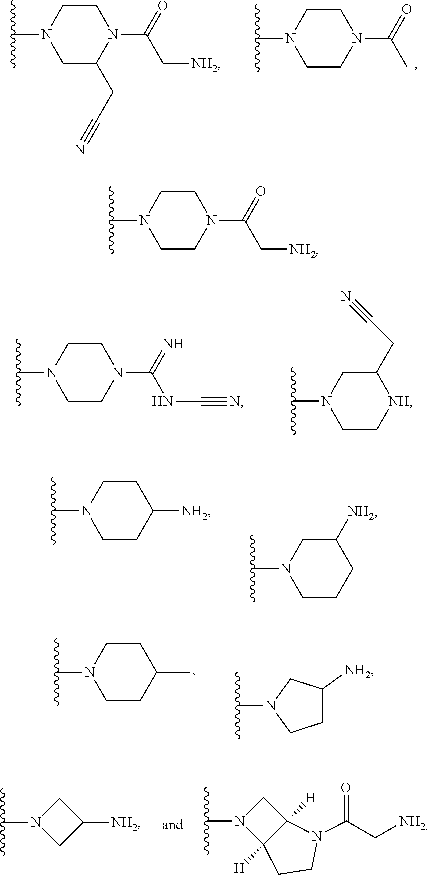

Embodiment I-118. The compound of any one of embodiments I-114 to I-117, wherein Ring A includes at least two nitrogen atoms and/or is substituted with a group including an amino moiety.

Embodiment I-119. The compound of any one of embodiments I-114 to I-118, wherein R⁶ is selected from:

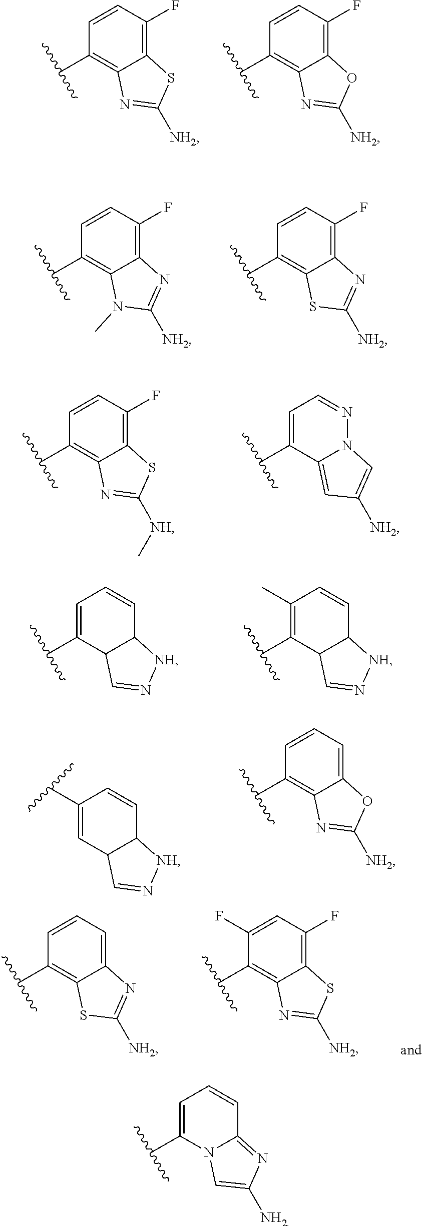

Embodiment I-120. The compound of embodiment I-119, wherein R⁶ is selected from:

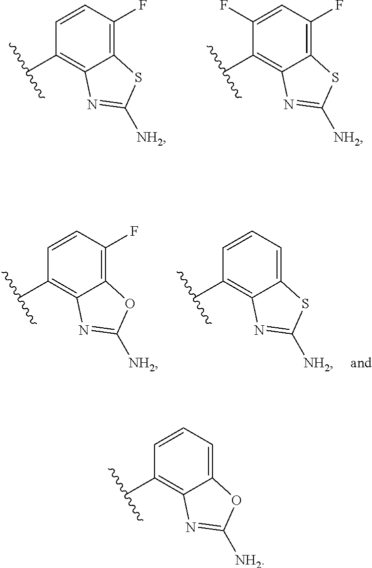 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; *c1cccc2oc(N)nc12]

Embodiment I-121. The compound of embodiment I-120, wherein R⁶ is:

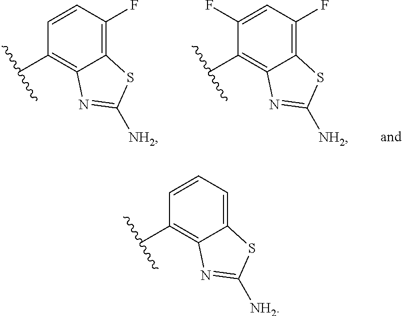 [SMILES: *c1ccc(F)c2sc(N)nc12 ; *c1c(F)cc(F)c2sc(N)nc12 ; *c1cccc2sc(N)nc12]

Embodiment I-122. The compound of any one of embodiments I-114 to I-121, wherein R⁴ is H.

Embodiment I-123. The compound of any one of embodiments I-114 to I-121, wherein R⁴ is a halogen.

Embodiment I-124. The compound of any one of embodiments I-114 to I-123, wherein R⁵ is a halogen.

Embodiment I-125. The compound of any one of embodiments I-114 to I-124, wherein R⁷ is a halogen.

Embodiment I-126. A compound according to Formula IB2:

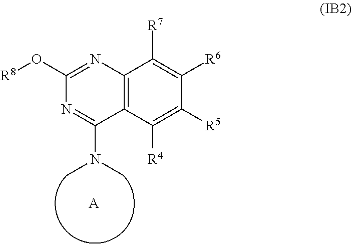

or a salt (e.g., pharmaceutically acceptable salt), ester, tautomer, prodrug, zwitterionic form, or stereoisomer thereof, wherein:
R¹ is —OR⁸; Ring A is a heterocycle that is unsubstituted or substituted with one or more R¹¹;
R⁴ is H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-127. The compound of embodiment I-126, wherein Ring A is a 4-6 membered heterocycle that is unsubstituted or substituted with one or more R¹¹.

Embodiment I-128. The compound of embodiment I-126, wherein Ring A has the structure:

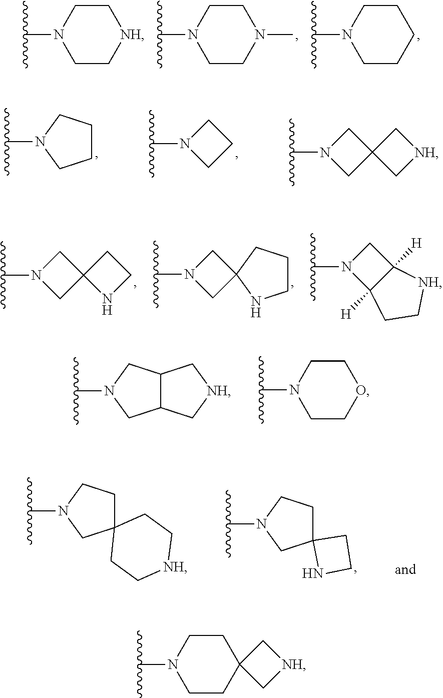

which ring is unsubstituted or substituted with one or more R¹¹.

Embodiment I-129. The compound of embodiment I-126, wherein Ring A has the structure:

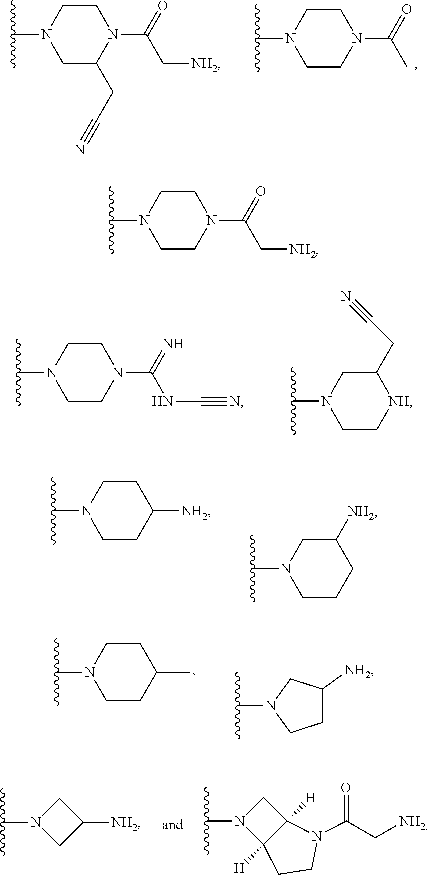

Embodiment I-130. The compound of any one of embodiments I-126 to I-129, wherein Ring A includes at least two nitrogen atoms and/or is substituted with a group including an amino moiety.

Embodiment I-131. The compound of any one of embodiments I-126 to I-130, wherein R⁸ comprises a 3-6 membered heterocycle that is unsubstituted or substituted with one or more R¹⁴.

Embodiment I-132. The compound of any one of embodiments I-126 to I-131, wherein R¹ is selected from:

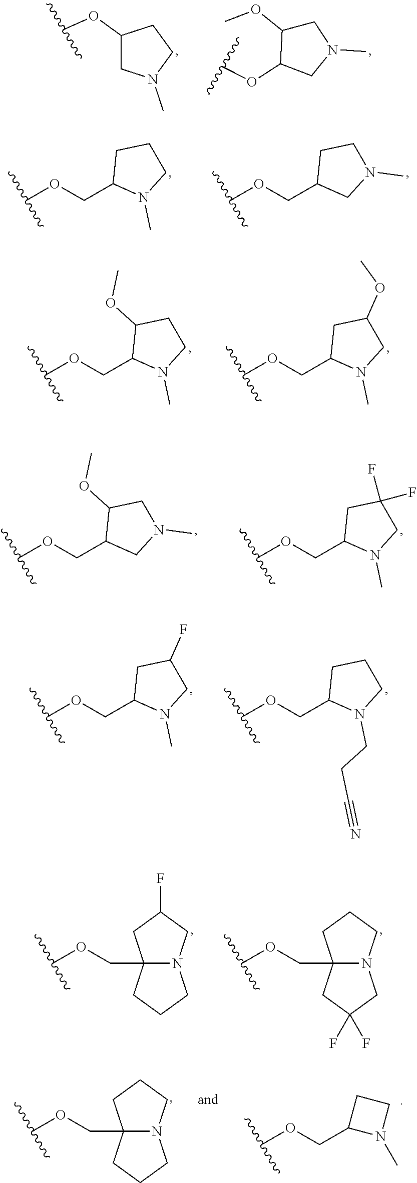

Embodiment I-133. The compound of any one of embodiments I-126 to I-131, wherein R¹ is selected from:

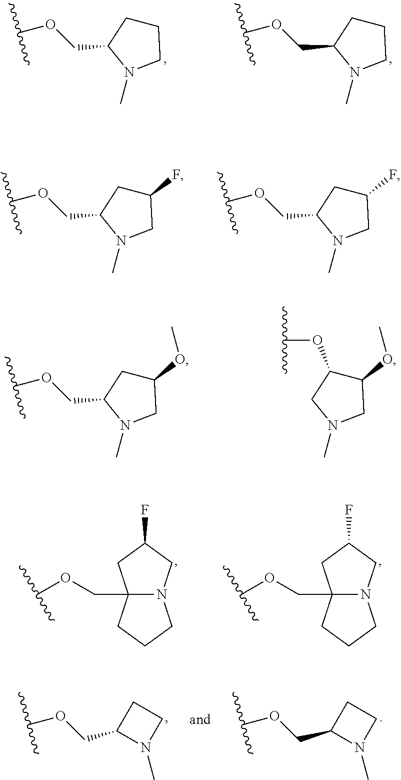

Embodiment I-134. The compound of any one of embodiments I-126 to I-133, wherein R⁶ is selected from:

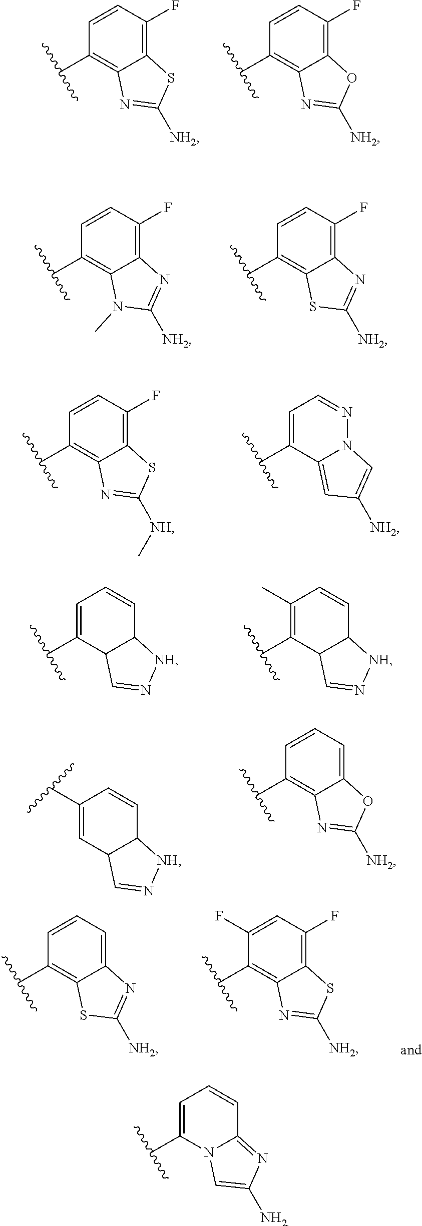

Embodiment I-135. The compound of embodiment I-134, wherein R⁶ is selected from:

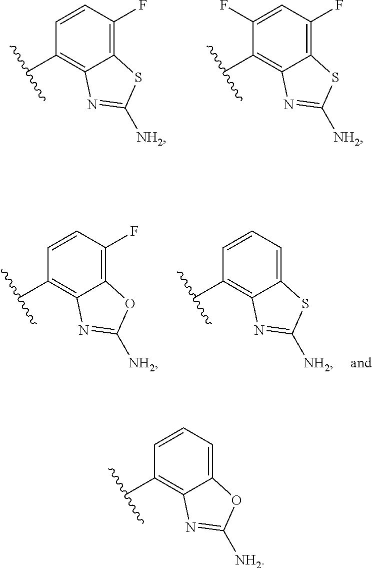 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; *c1cccc2oc(N)nc12]

Embodiment I-136. The compound of embodiment I-135, wherein R⁶ is:

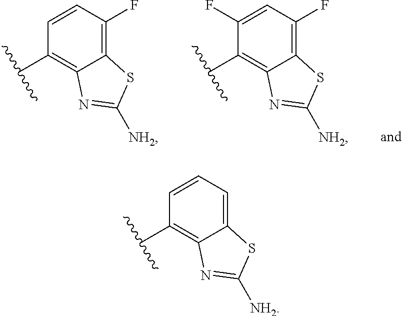 [SMILES: *c1ccc(F)c2sc(N)nc12 ; *c1c(F)cc(F)c2sc(N)nc12 ; *c1cccc2sc(N)nc12]

Embodiment I-137. The compound of any one of embodiments I-126 to I-136, wherein R⁴ is H.

Embodiment I-138. The compound of any one of embodiments I-126 to I-136, wherein R⁴ is a halogen.

Embodiment I-139. The compound of any one of embodiments I-126 to I-138, wherein R⁵ is a halogen.

Embodiment I-140. The compound of any one of embodiments I-126 to I-139, wherein R⁷ is a halogen.

Embodiment I-141. A compound according to Formula IC:

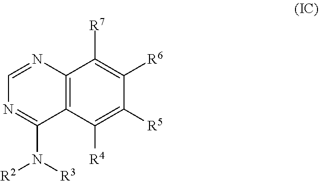

or a salt (e.g., pharmaceutically acceptable salt), ester, tautomer, prodrug, zwitterionic form, or stereoisomer thereof, wherein:
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-142. The compound of embodiment I-141, wherein R³ is selected from a carbocycle, and a heterocycle, wherein the carbocycle or heterocycle is unsubstituted or substituted with one or more R¹⁰.

Embodiment I-143. The compound of embodiment I-141, wherein R³ is selected from:

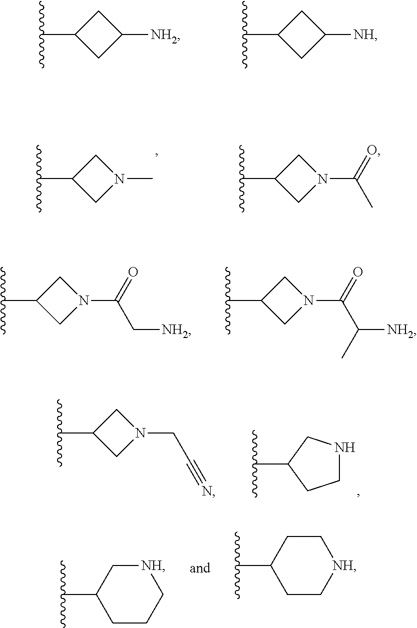

any of which is unsubstituted or substituted with one or more R¹⁰.

Embodiment I-144. The compound of any one of embodiments I-141 to I-143, wherein R³ is selected from C₁₋₆ alkyl that is substituted with one or more R⁹.

Embodiment I-145. The compound of embodiment I-144, wherein R³ is selected from:

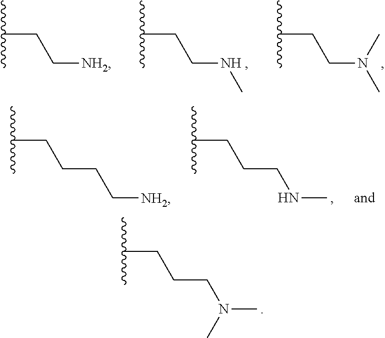

Embodiment I-146. The compound of any one of embodiments I-141 to I-145, wherein R³ is selected from C₁₋₆alkyl-N(R¹⁷)C(O)C₁₋₆alkylN(R¹⁷)₂.

Embodiment I-147. The compound of any one of embodiments I-141 to I-146, wherein R³ includes an amino moiety.

Embodiment I-148. The compound of embodiment I-141, wherein R² and R³, together with the atom to which they are attached, form a 4-6 membered heterocycle that is unsubstituted or substituted with one or more R¹¹.

Embodiment I-149. The compound of embodiment I-141, wherein R² and R³, together with the atom to which they are attached, form a heterocycle having the structure:

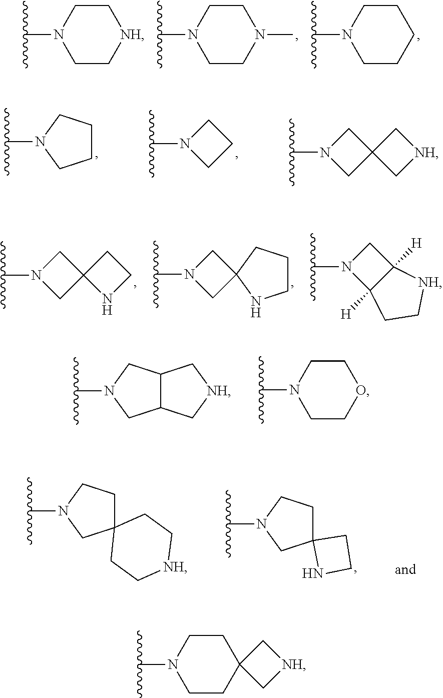

which ring is unsubstituted or substituted with one or more R¹¹.

Embodiment I-150. The compound of embodiment I-141, wherein R² and R³, together with the atom to which they are attached, form a heterocycle having the structure:

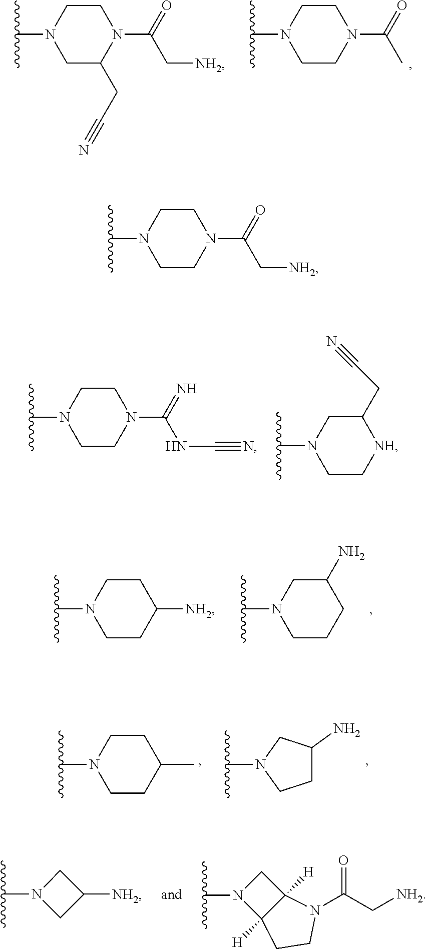

Embodiment I-151. The compound of any one of embodiments I-148 to I-150, wherein R² and R³, together with the atom to which they are attached, form a heterocycle that (i) comprises an additional nitrogen atom or (ii) is substituted with a group including an amino moiety.

Embodiment I-152. The compound of any one of embodiments I-141 to I-151, wherein R⁶ is selected from:

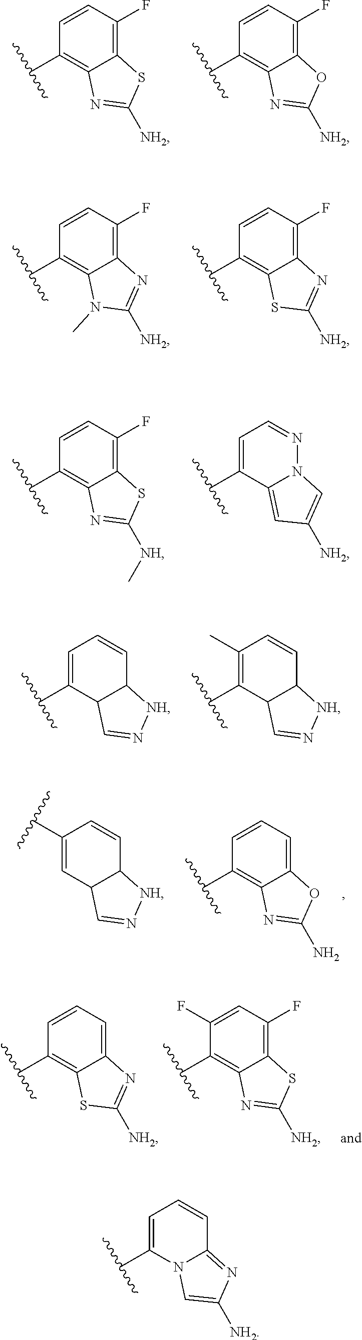

Embodiment I-153. The compound of embodiment I-152, wherein R⁶ is selected from:

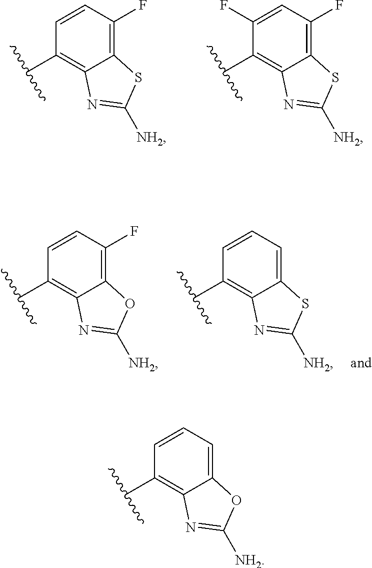 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; *c1cccc2oc(N)nc12]

Embodiment I-154. The compound of embodiment I-153, wherein R⁶ is:

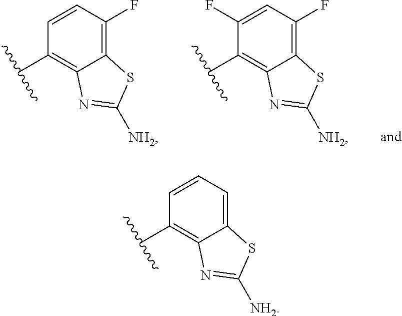 [SMILES: *c1ccc(F)c2sc(N)nc12 ; *c1c(F)cc(F)c2sc(N)nc12 ; *c1cccc2sc(N)nc12]

Embodiment I-155. The compound of any one of embodiments I-141 to I-154, wherein R⁴ is H.

Embodiment I-156. The compound of any one of embodiments I-141 to I-154, wherein R⁴ is a halogen.

Embodiment I-157. The compound of any one of embodiments I-141 to I-156, wherein R⁵ is a halogen.

Embodiment I-158. The compound of any one of embodiments I-141 to I-157, wherein R⁷ is a halogen.

Embodiment I-159. A compound according to Formula ID:

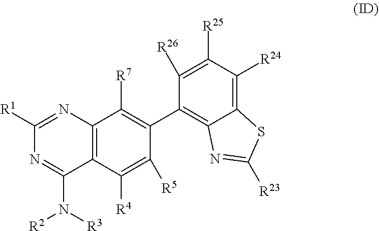

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.
R²³ is selected from —N(R¹²)₂ and C₁₋₆alkyl-N(R¹²)₂;
R²⁴, R²⁵, and R²⁶ are independently selected from H, halogen, —OR¹², and C₁₋₆alkyl, wherein any C₁₋₆alkyl is unsubstituted or substituted with one or more R¹¹; and
each R²⁷ is independently selected from —OH, —OC₁₋₆alkyl, —CN, —NH₂, —NHC₁₋₆alkyl, and halogen.

Embodiment I-160. The compound of embodiment I-159, wherein R³ is selected from a carbocycle, and a heterocycle, wherein the carbocycle or heterocycle is unsubstituted or substituted with one or more R¹⁰.

Embodiment I-161. The compound of embodiment I-159, wherein R³ is selected from:

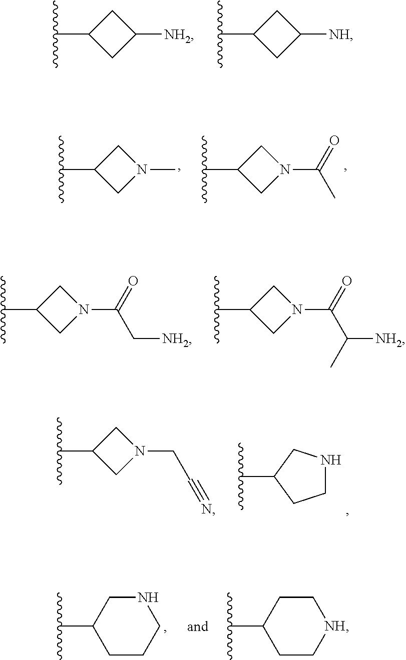

any of which is unsubstituted or substituted with one or more R¹⁰.

Embodiment I-162. The compound of embodiment I-159, wherein R³ is selected from C₁₋₆ alkyl that is substituted with one or more R⁹.

Embodiment I-163. The compound of embodiment I-162, wherein R³ is selected from:

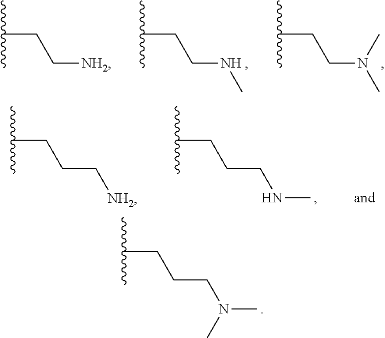

Embodiment I-164. The compound of embodiment I-159, wherein R³ is selected from C₁₋₆alkyl-N(R¹⁷)C(O)C₁₋₆alkylN(R¹⁷)₂.

Embodiment I-165. The compound of any one of embodiments I-159 to I-164, wherein R³ comprises an amino moiety.

Embodiment I-166. The compound of embodiment I-159, wherein R² and R³, together with the atom to which they are attached, form a 4-6 membered heterocycle that is unsubstituted or substituted with one or more R¹¹.

Embodiment I-167. The compound of embodiment I-159, wherein R² and R³, together with the atom to which they are attached, form a heterocycle having the structure:

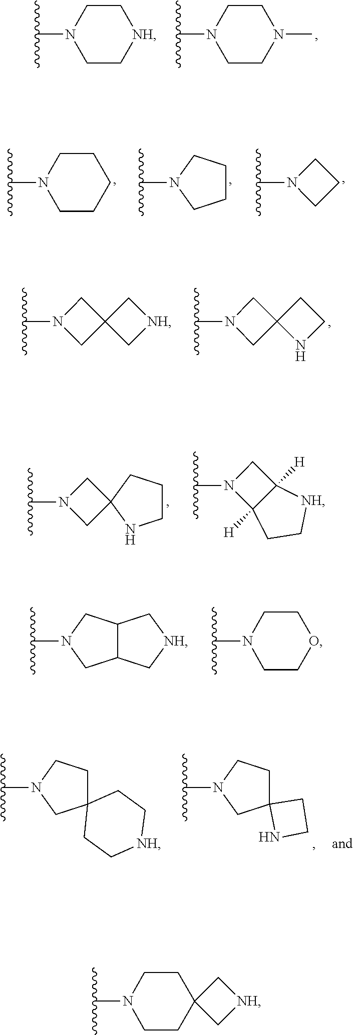

which ring is unsubstituted or substituted with one or more R¹¹.

Embodiment I-168. The compound of embodiment I-159, wherein R² and R³, together with the atom to which they are attached, form a heterocycle having the structure:

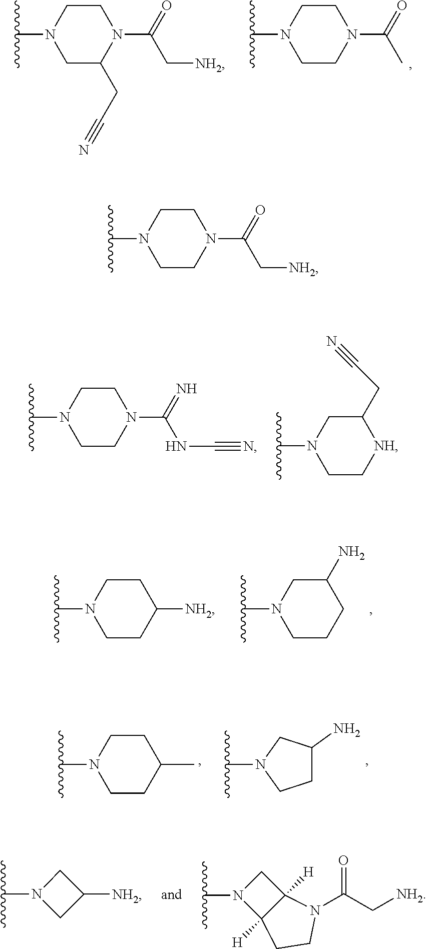

Embodiment I-169. The compound of any one of embodiments I-166 to I-168, wherein R² and R³, together with the atom to which they are attached, form a heterocycle that comprises (i) an additional nitrogen atom or (ii) is substituted with a group including an amino moiety.

Embodiment I-170. The compound of any one of embodiments I-159 to I-169, wherein R¹ is H.

Embodiment I-171. The compound of any one of embodiments I-159 to I-169, wherein R¹ is —OR⁸.

Embodiment I-172. The compound of embodiment I-171, wherein R⁸ comprises a 3-6 membered heterocycle that is unsubstituted or substituted with one or more R¹⁴.

Embodiment I-173. The compound of any one of embodiments I-159 to I-169, wherein R¹ is selected from:

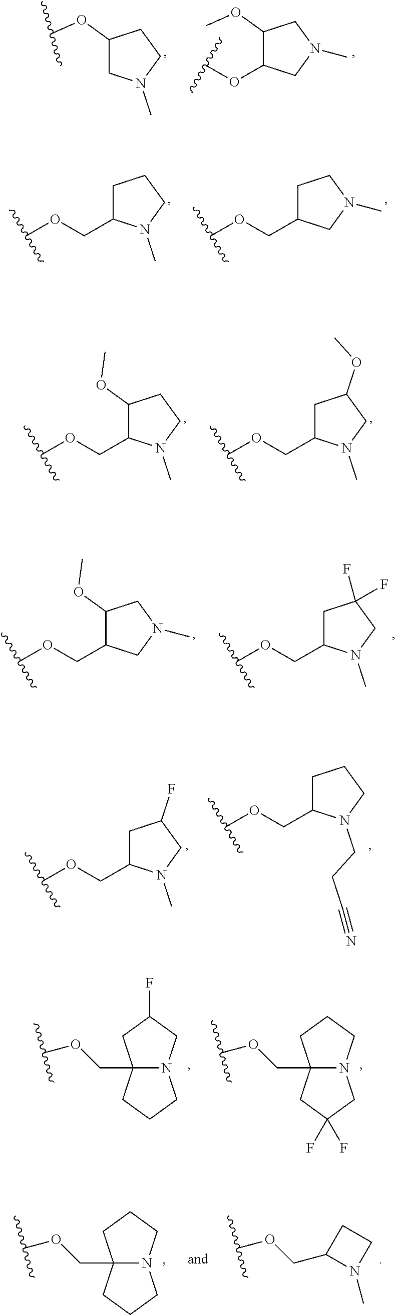

Embodiment I-174. The compound of any one of embodiments I-159 to I-169, wherein R¹ is selected from:

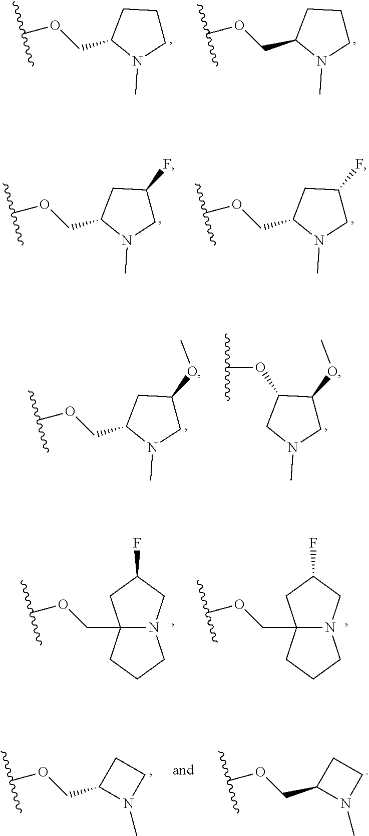

Embodiment I-175. The compound of any one of embodiments I-159 to I-169, wherein R¹ is a 4-6 membered heterocycle comprising a nitrogen atom that is unsubstituted or substituted with one or more R¹⁶.

Embodiment I-176. The compound of embodiment I-175, wherein R¹ is selected from:

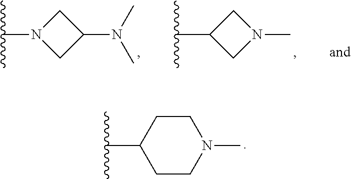

Embodiment I-177. The compound of any one of embodiments I-159 to I-176, wherein R⁴ is H.

Embodiment I-178. The compound of any one of embodiments I-159 to I-176, wherein R⁴ is a halogen.

Embodiment I-179. The compound of any one of embodiments I-159 to I-178, wherein R⁵ is a halogen.

Embodiment I-180. The compound of any one of embodiments I-159 to I-179, wherein R⁷ is a halogen.

Embodiment I-181. The compound of any one of embodiments I-159 to I-180, wherein R²³ is —N(R¹²)₂.

Embodiment I-182. The compound of embodiment I-181, wherein R²³ is —NH₂.

Embodiment I-183. The compound of any one of embodiments I-159 to I-182, wherein R²⁴ is a halogen.

Embodiment I-184. The compound of any one of embodiments I-159 to I-183, wherein R²⁵ and R²⁶ are H.

Embodiment I-185. A compound according to Formula II:

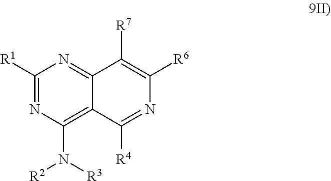

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment I-186. The compound of embodiment I-185, wherein R³ is selected from a carbocycle, and a heterocycle, wherein the carbocycle or heterocycle is unsubstituted or substituted with one or more R¹⁰.

Embodiment I-187. The compound of embodiment I-186, wherein R³ is selected from:

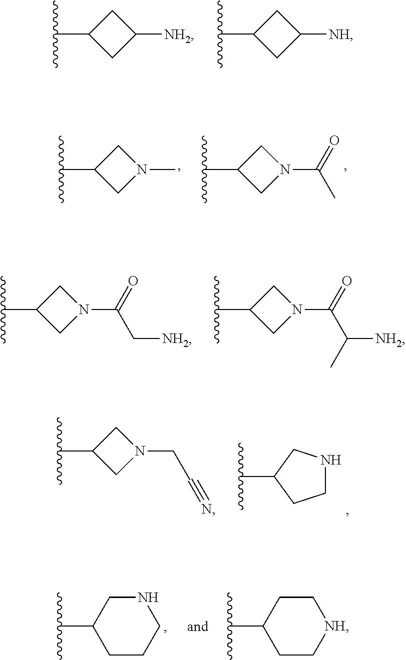

any of which is unsubstituted or substituted with one or more R¹⁰.

Embodiment I-188. The compound of embodiment I-185, wherein R³ is selected from C₁₋₆ alkyl that is substituted with one or more R⁹.

Embodiment I-189. The compound of embodiment I-188, wherein R³ is selected from:

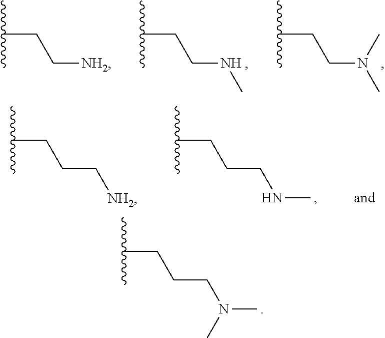

Embodiment I-190. The compound of embodiment I-185, wherein R³ is selected from C₁₋₆alkyl-N(R¹⁷)C(O)C₁₋₆alkylN(R¹⁷)₂.

Embodiment I-191. The compound of any one of embodiments I-185 to I-190, wherein R³ includes an amino moiety.

Embodiment I-192. The compound of embodiment I-185, wherein R² and R³, together with the atom to which they are attached, form a 4-6 membered heterocycle that is unsubstituted or substituted with one or more R¹¹.

Embodiment I-193. The compound of embodiment I-185, wherein R² and R³, together with the atom to which they are attached, form a heterocycle having the structure:

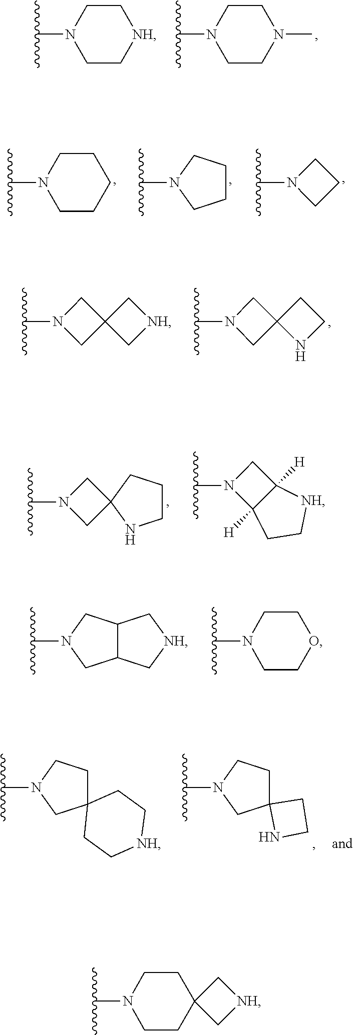

which ring is unsubstituted or substituted with one or more R¹¹.

Embodiment I-194. The compound of embodiment I-185, wherein R² and R³, together with the atom to which they are attached, form a heterocycle having the structure:

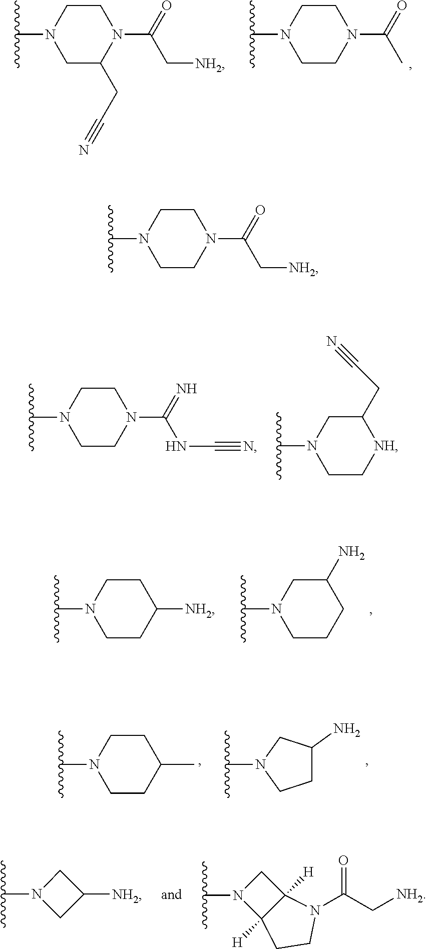

Embodiment I-195. The compound of any one of embodiments I-192 to I-194, wherein R² and R³, together with the atom to which they are attached, form a heterocycle that (i) comprises an additional nitrogen atom and/or (ii) is substituted with a group including an amino moiety.

Embodiment I-196. The compound of any one of embodiments I-185 to I-195, wherein R¹ is H.

Embodiment I-197. The compound of any one of embodiments I-185 to I-195, wherein R¹ is —OR⁸.

Embodiment I-198. The compound of embodiment I-197, wherein R⁸ comprises a 3-6 membered heterocycle that is unsubstituted or substituted with one or more R¹⁴.

Embodiment I-199. The compound of any one of embodiments I-185 to I-195, wherein R¹ is selected from:

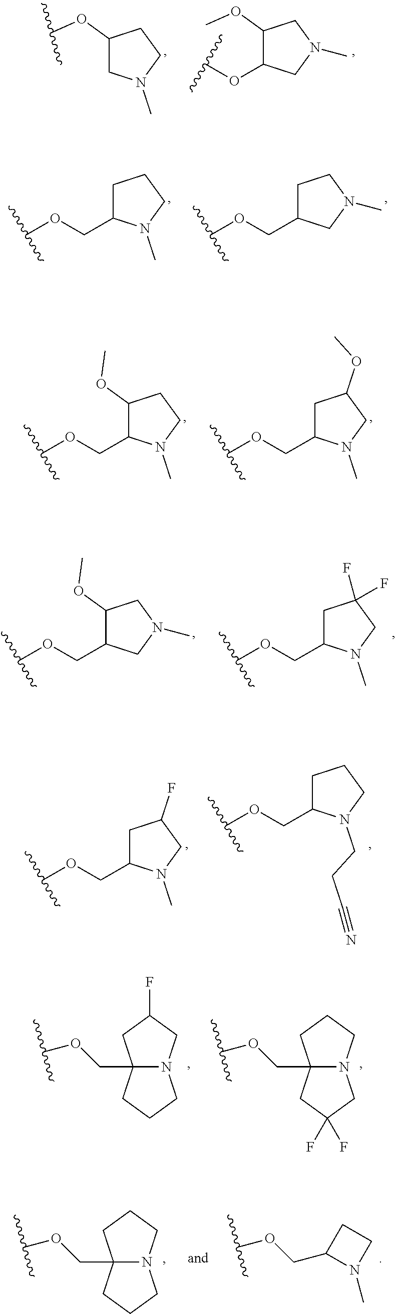

Embodiment I-200. The compound of any one of embodiments I-185 to I-195, wherein R¹ is selected from:

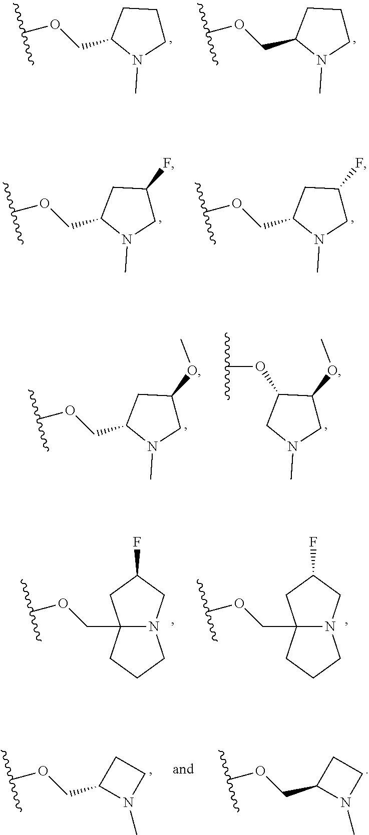

Embodiment I-201. The compound of any one of embodiments I-185 to I-195, wherein R¹ is a 4-6 membered heterocycle comprising a nitrogen atom that is unsubstituted or substituted with one or more R¹⁶.

Embodiment I-202. The compound of embodiment I-201, wherein R¹ is selected from:

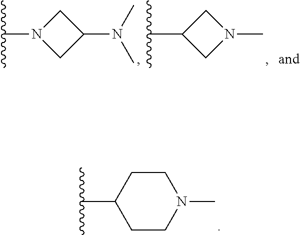

Embodiment I-203. The compound of any one of embodiments I-185 to I-202, wherein R⁶ is selected from:

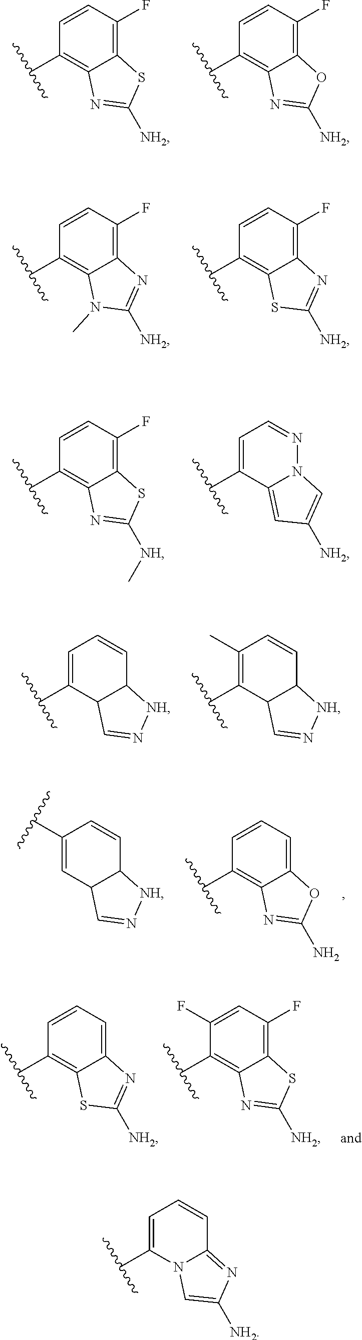

Embodiment I-204. The compound of embodiment I-203, wherein R⁶ is selected from:

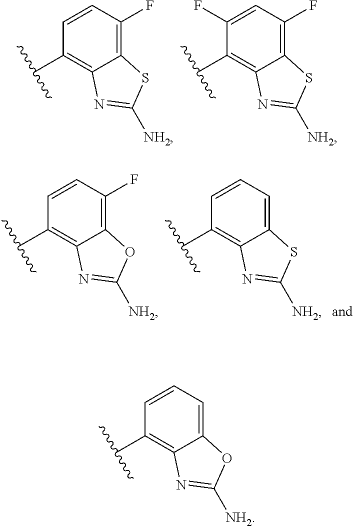

Embodiment I-205. The compound of embodiment I-204 wherein R is:

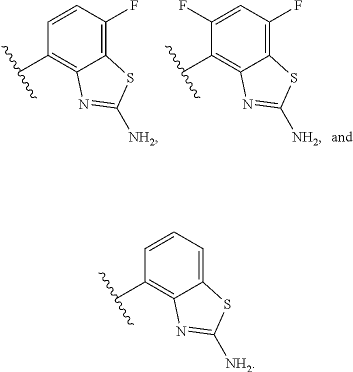 [SMILES: *c1ccc(F)c2sc(N)nc12 ; *c1c(F)cc(F)c2sc(N)nc12 ; *c1cccc2sc(N)nc12]

Embodiment I-206. The compound of any one of embodiments I-185 to I-205, wherein R⁴ is H.

Embodiment I-207. The compound of any one of embodiments I-185 to I-205, wherein R⁴ is a halogen.

Embodiment I-208. The compound of any one of embodiments I-185 to I-207, wherein R⁷ is a halogen.

Embodiment I-209. A compound shown in any one of Tables 2-4, or a salt (e.g., pharmaceutically acceptable salt), ester, tautomer, prodrug, zwitterionic form, or stereoisomer thereof.

Embodiment I-210. The compound of embodiment I-209, wherein the compound is shown in Table 2.

Embodiment I-211. The compound of embodiment I-209, wherein the compound is shown in Table 3.

Embodiment I-212. The compound of embodiment I-209, wherein the compound is shown in Table 4.

Embodiment I-213. A pharmaceutical composition comprising a compound of any one of embodiments I-1 to I-212, or a salt (e.g., pharmaceutically acceptable salt), ester, tautomer, prodrug, zwitterionic form, or stereoisomer thereof, and a pharmaceutically acceptable excipient.

Embodiment I-214. A compound of any one of embodiments I-1 to I-212, or a salt (e.g., pharmaceutically acceptable salt), ester, tautomer, prodrug, zwitterionic form, or stereoisomer thereof, for use as a medicament.

Embodiment I-215. The compound of embodiment I-214, wherein the medicament is useful in the prevention or treatment of a disease, disorder, or condition ameliorated by the inhibition of KRAS having a G12D mutation.

Embodiment I-216. The compound of embodiment I-214 or I-215, wherein the medicament is useful in the prevention or treatment of a cancer.

Embodiment I-217. The compound of embodiment I-216, wherein the cancer is selected from the group consisting of pancreatic cancer, colorectal cancer, and lung cancer.

Embodiment I-218. A compound of any one of embodiments I-1 to I-212, or a salt (e.g., pharmaceutically acceptable salt), ester, tautomer, prodrug, zwitterionic form, or stereoisomer thereof, for use in the treatment of a disease, disorder, or condition.

Embodiment I-219. The compound of embodiment I-218, wherein the disease, disorder, or condition is a cancer.

Embodiment I-220. The compound of embodiment I-219, wherein the cancer is selected from the group consisting of pancreatic cancer, colorectal cancer, and lung cancer.

Embodiment I-221. The compound of any one of embodiments I-218 to I-220, wherein the compound is used in the treatment of a disease, disorder, or condition in a subject in need thereof.

Embodiment I-222. A compound of any one of embodiments I-1 to I-212, or a salt (e.g., pharmaceutically acceptable salt), ester, tautomer, prodrug, zwitterionic form, or stereoisomer thereof, for use in the manufacture of a medicament.

Embodiment I-223. The compound of embodiment I-222, wherein the medicament is useful in the prevention or treatment of a disease, disorder, or condition ameliorated by the inhibition of KRAS having a G12D mutation.

Embodiment I-224. The compound of embodiment I-222 or I-223, wherein the medicament is useful in the treatment of a cancer.

Embodiment I-225. The compound of embodiment I-224, wherein the cancer is selected from the group consisting of pancreatic cancer, colorectal cancer, and lung cancer.

Embodiment I-226. A method, comprising administering a therapeutically effective amount of a compound of any one of embodiments I-1 to I-212, or a salt (e.g., pharmaceutically acceptable salt), ester, tautomer, prodrug, zwitterionic form, or stereoisomer thereof, to a subject in need thereof.

Embodiment I-227. The method of embodiment I-226, wherein the subject has a disease, disorder, or condition ameliorated by the inhibition of KRAS having a G12D mutation.

Embodiment I-228. The method of embodiment I-226 or I-227, wherein the subject has a cancer.

Embodiment I-229. The method of embodiment I-228, wherein the subject was previously diagnosed with the cancer.

Embodiment I-230. The method of embodiment I-228, wherein the subject has previously undergone a treatment regimen for the cancer.

Embodiment I-231. The method of embodiment I-228, wherein the subject has previously entered remission from the cancer.

Embodiment I-232. The method of any one of embodiments I-228 to I-230, wherein the cancer is selected from the group consisting of pancreatic cancer, colorectal cancer, and lung cancer.

Embodiment I-233. The method of any one of embodiments I-226 to I-232, wherein the compound, or the salt, ester, tautomer, prodrug, zwitterionic form, or stereoisomer thereof, is administered in combination with an additional therapeutic agent.

Embodiment I-234. The use of a compound of any one of embodiments I-1 to I-212, or a salt (e.g., pharmaceutically acceptable salt), ester, tautomer, prodrug, zwitterionic form, or stereoisomer thereof, for the manufacture of a medicament for the treatment of a cancer.

Embodiment I-235. The use of embodiment I-234, wherein the cancer is selected from the group consisting of pancreatic cancer, colorectal cancer, and lung cancer.

Embodiment I-236. A method, comprising contacting a KRAS protein with a compound of any one of embodiments I-1 to I-212, or a salt (e.g., pharmaceutically acceptable salt), ester, tautomer, prodrug, zwitterionic form, or stereoisomer thereof.

Embodiment I-237. The method of embodiment I-236, wherein contacting the KRAS protein with the compound modulates KRAS.

Embodiment I-238. The method of embodiment I-236 or I-237, wherein the KRAS protein has a G12D mutation.

Embodiment I-239. The method of any one of embodiments I-236 to I-238, wherein the KRAS protein is in an active state.

Embodiment I-240. The method of any one of embodiments I-236 to I-238, wherein the KRAS protein is in an inactive state.

Embodiment I-241. The compound of any one of embodiments I-1 to I-212, or a salt (e.g., a pharmaceutically acceptable salt) thereof.

Embodiment II-1. A compound represented by Formula I:

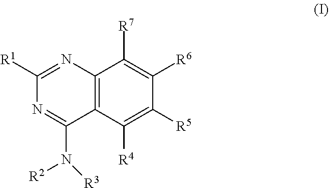

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-2. The compound of embodiment II-1, wherein R¹ is —OR⁸.

Embodiment II-3. The compound of embodiment II-2, wherein R⁸ is a heterocycle.

Embodiment II-4. The compound of embodiment II-3, wherein R⁸ is an alkylheterocycle.

Embodiment II-5. The compound of embodiment II-3 or II-4, wherein R⁸ comprises a heterocycle comprising at least one nitrogen atom.

Embodiment II-6. The compound of any one of embodiments II-3 to II-5, wherein the heterocycle comprises one or R¹⁶ substituents.

Embodiment II-7. The compound of embodiment II-6, wherein at least one R¹⁶ is —OR¹², where R¹² is independently selected from C₁₋₆ alkyl, C₂₋₆ alkenyl, and H.

Embodiment II-8. The compound of embodiment II-7, wherein at least one R¹⁶ is —OCH₃.

Embodiment II-9. The compound of embodiment II-1, wherein R¹ is selected from:

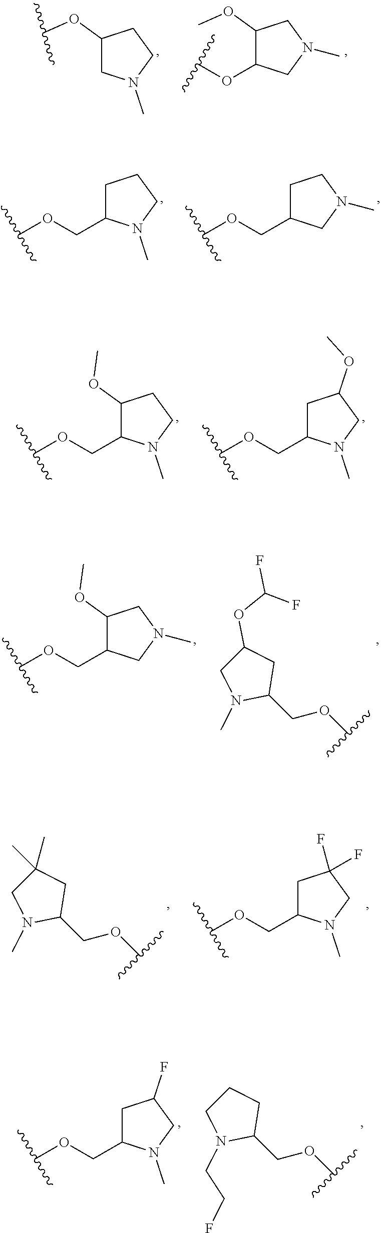

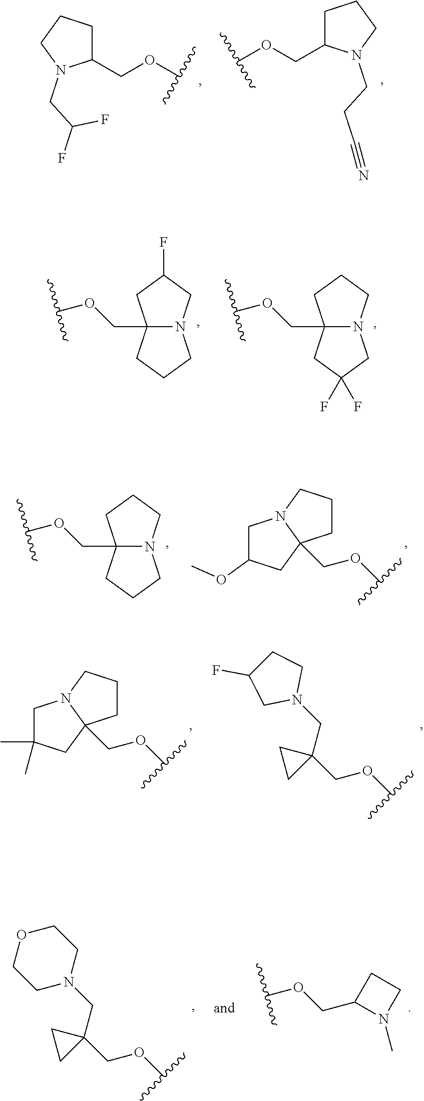

Embodiment II-10. The compound of embodiment II-1, wherein R¹ is selected from:

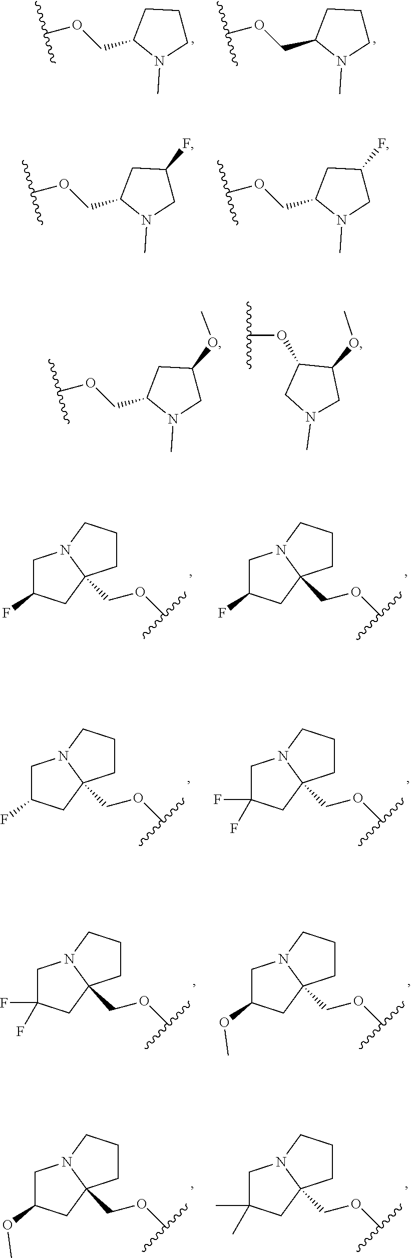

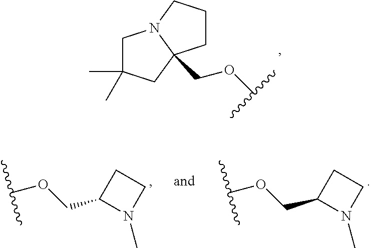

Embodiment II-11. The compound of embodiment II-1, wherein R¹ is a 4-6 membered heterocycle comprising a nitrogen atom, which heterocycle is unsubstituted or substituted with one or more R¹⁶.

Embodiment II-12. The compound of embodiment II-11, wherein R¹ is selected from:

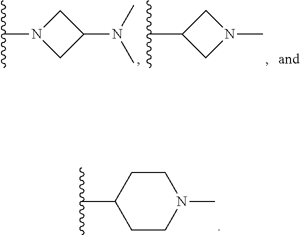

Embodiment II-13. The compound of embodiment II-1, wherein R¹ is H.

Embodiment II-14. The compound of any one of embodiments II-1 to II-13, wherein R² is H.

Embodiment II-15. The compound of any one of embodiments II-1 to II-13, wherein R² is C₁₋₆ alkyl.

Embodiment II-16. The compound of any one of embodiments II-1 to II-15, wherein R³ is C₁₋₆ alkyl, which C₁₋₆alkyl is substituted with one or more R⁹.

Embodiment II-17. The compound of embodiment II-16, wherein R³ is C₁₋₆ alkyl substituted with —N(R¹⁷)₂, where each R¹⁷ is independently selected from C₁₋₆ alkyl and H.

Embodiment II-18. The compound of embodiment II-17, wherein R³ is C₁₋₆ alkyl substituted with —NH₂.

Embodiment II-19. The compound of any one of embodiments II-1 to II-15, wherein R³ is selected from:

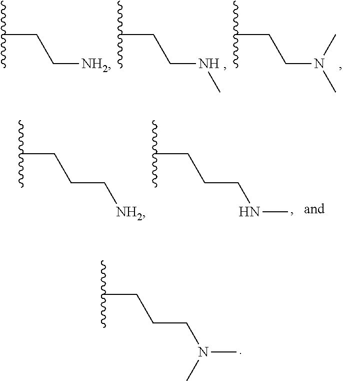

Embodiment II-20. The compound of any one of embodiments II-1 to II-15, wherein R³ is a carbocycle, which carbocycle is unsubstituted or substituted with one or more R¹⁰.

Embodiment II-21. The compound of embodiment II-20, wherein R³ is a carbocycle that is substituted with —N(R¹⁹)₂, where each R¹⁹ is independently selected from unsubstituted or substituted C₁₋₆ alkyl and H.

Embodiment II-22. The compound of embodiment II-21, wherein R³ is a carbocycle that is substituted with —NH₂.

Embodiment II-23. The compound of any one of embodiments II-1 to II-15, wherein R³ is selected from C₁₋₆alkyl-N(R¹⁷)C(O)C₁₋₆alkylN(R¹⁷)₂.

Embodiment II-24. The compound of any one of embodiment II-1 to II-15, wherein R³ is a heterocycle, which heterocycle is unsubstituted or substituted with one or more R¹⁰.

Embodiment II-25. The compound of embodiment II-24, wherein the heterocycle comprises one or more nitrogen atoms.

Embodiment II-26. The compound of embodiment II-24 or II-25, wherein R³ is a heterocycle that is substituted with —N(R¹⁹)₂, where each R¹⁹ is independently selected from unsubstituted or substituted C₁₋₆ alkyl and H.

Embodiment II-27. The compound of embodiment II-26, wherein R³ is a heterocycle that is substituted with —NH₂.

Embodiment II-28. The compound of any one of embodiments II-1 to II-15, wherein R³ is selected from:

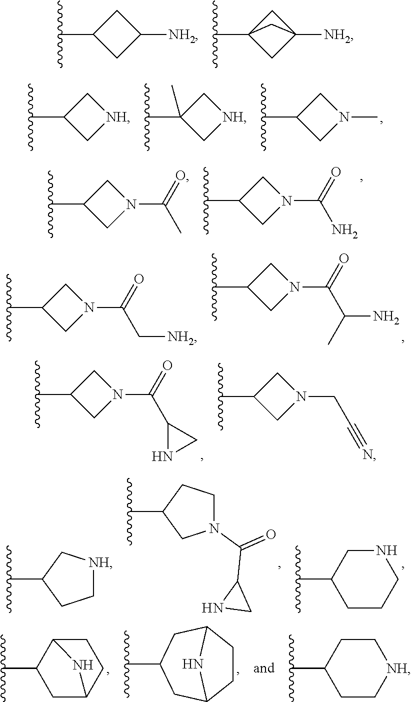

any of which is unsubstituted or substituted with one or more R¹⁰.

Embodiment II-29. The compound of any one of embodiments II-15 to II-28, wherein R² or R³ comprises an amino moiety.

Embodiment II-30. The compound of any one of embodiments II-1 to II-13, wherein R² and R³, together with the atom (e.g., nitrogen atom) to which they are attached, form a heterocycle that is unsubstituted or substituted with one or more R¹¹.

Embodiment II-31. The compound of embodiment II-30, wherein R² and R³, together with the atom to which they are attached, form a heterocycle having the structure:

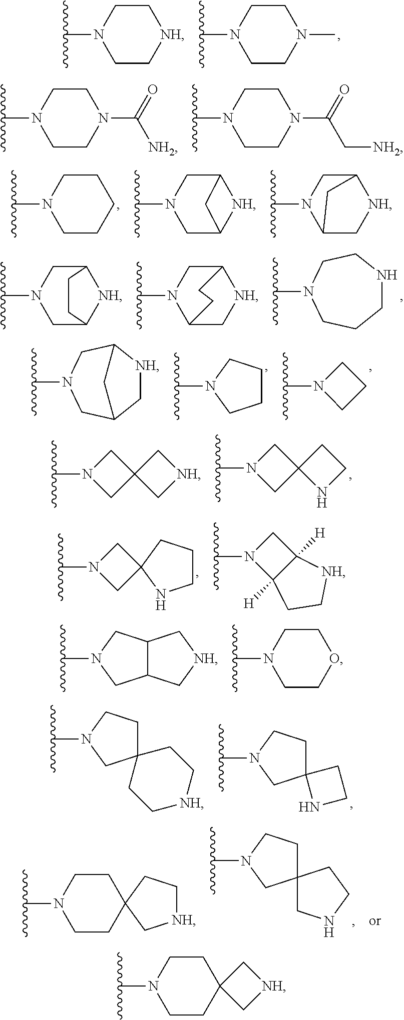

any of which is unsubstituted or substituted with one or more R¹¹.

Embodiment II-32. The compound of embodiment II-30, wherein R² and R³, together with the atom to which they are attached, form a heterocycle having the structure:

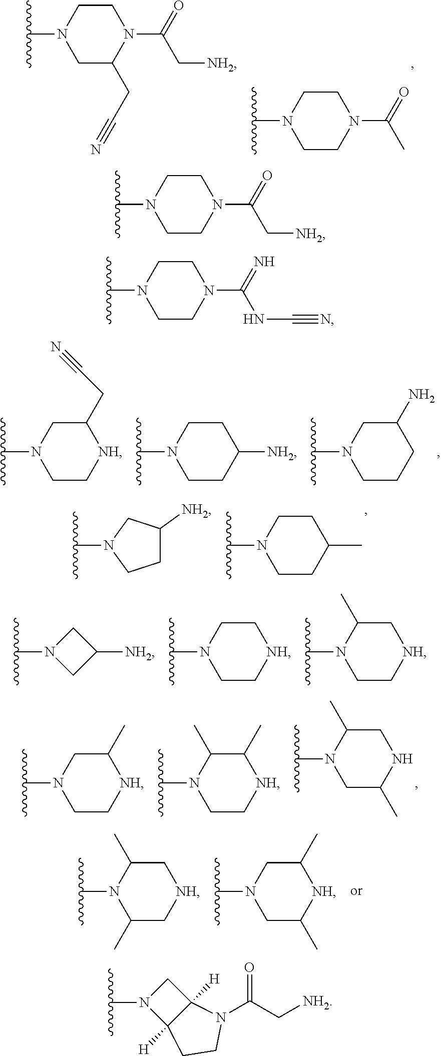

Embodiment II-33. The compound of any one of embodiments II-30 to II-32, wherein R² and R³, together with the nitrogen atom to which they are attached, form a heterocycle that (i) comprises an additional nitrogen atom or (ii) is substituted with a group comprising an amino moiety.

Embodiment II-34. The compound of any one of embodiments II-1 to II-33, wherein R⁴ is hydrogen.

Embodiment II-35. The compound of any one of embodiments II-1 to II-33, wherein R⁴ is a halogen.

Embodiment II-36. The compound of any one of embodiments II-1 to II-35, wherein R⁵ is a halogen.

Embodiment II-37. The compound of any one of embodiments II-1 to II-35, wherein R⁵ is selected from C₁₋₆alkyl that is unsubstituted or substituted with one or more R¹³.

Embodiment II-38. The compound of any one of embodiments II-1 to II-35, wherein R⁵ is selected from a 3-6 membered carbocycle and a 3-6 membered heterocycle, wherein the carbocycle and heterocycle is unsubstituted or substituted with one or more R¹⁴.

Embodiment II-39. The compound of any one of embodiments II-1 to II-38, wherein R⁷ is a halogen.

Embodiment II-40. The compound of any one of embodiments II-1 to II-39, wherein R⁶ is selected from:

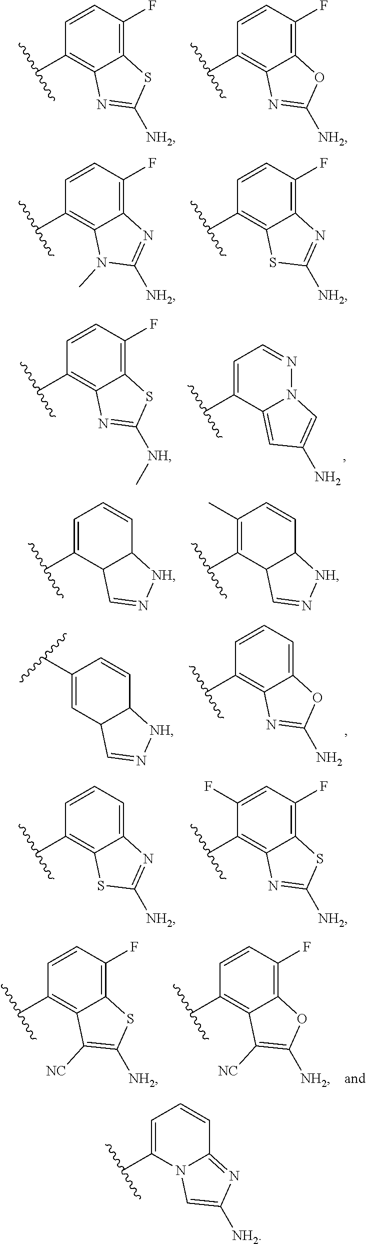

Embodiment II-41. The compound of embodiment II-40, wherein R⁶ is selected from:

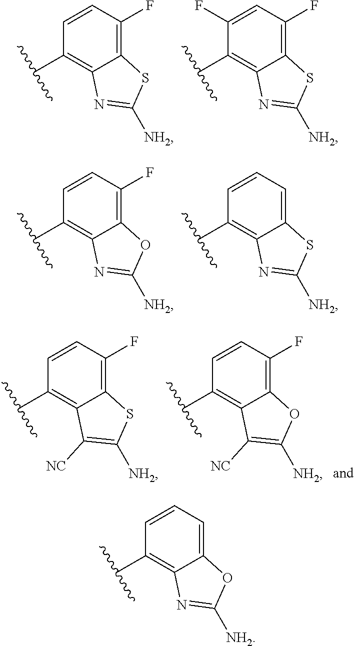

Embodiment II-42. The compound of embodiment II-41, wherein R⁶ is:

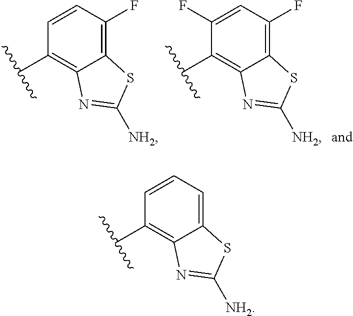 [SMILES: *c1ccc(F)c2sc(N)nc12 ; *c1c(F)cc(F)c2sc(N)nc12 ; *c1cccc2sc(N)nc12]

Embodiment II-43. A compound represented by Formula IA:

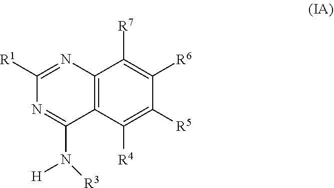

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
R² is H;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-44. The compound of embodiment II-43, wherein R³ is selected from a carbocycle and a heterocycle, wherein the carbocycle or heterocycle is unsubstituted or substituted with one or more R¹⁰.

Embodiment II-45. The compound of embodiment II-44, wherein R³ is a 3-6 membered carbocycle or heterocycle that is unsubstituted or substituted with one or more R¹⁰.

Embodiment II-46. The compound of embodiment II-45, wherein R³ is selected from:

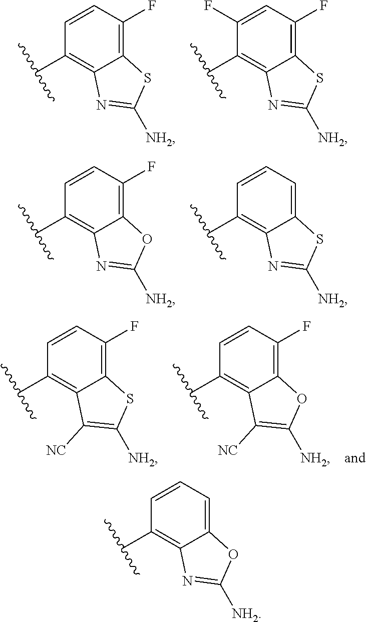

any of which is unsubstituted or substituted with one or more R¹⁰.

Embodiment II-47. The compound of embodiment II-43, wherein R³ is selected from C₁₋₆ alkyl that is substituted with one or more R⁹.

Embodiment II-48. The compound of embodiment II-47, wherein R³ is selected from C₁₋₆alkyl-N(R¹⁷)₂.

Embodiment II-49. The compound of embodiment II-47 or II-48, wherein R³ is selected from:

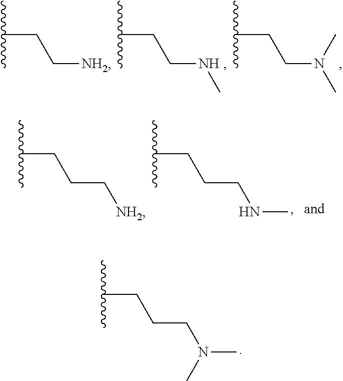

Embodiment II-50. The compound of embodiment II-47, wherein R³ is selected from C₁₋₆alkyl-N(R¹⁷)C(O)C₁₋₆alkylN(R¹⁷)₂.

Embodiment II-51. The compound of any one of embodiments II-43 to II-50, wherein R³ comprises an amino moiety.

Embodiment II-52. The compound of any one of embodiments II-43 to II-51, wherein R¹ is H.

Embodiment II-53. The compound of any one of embodiments II-43 to II-52, wherein R¹ is —OR⁸.

Embodiment II-54. The compound of embodiment II-53, wherein R⁸ comprises a 3-8 membered heterocycle that is unsubstituted or substituted with one or more R¹⁶.

Embodiment II-55. The compound of embodiment II-53 or II-54, wherein R¹ is selected from:

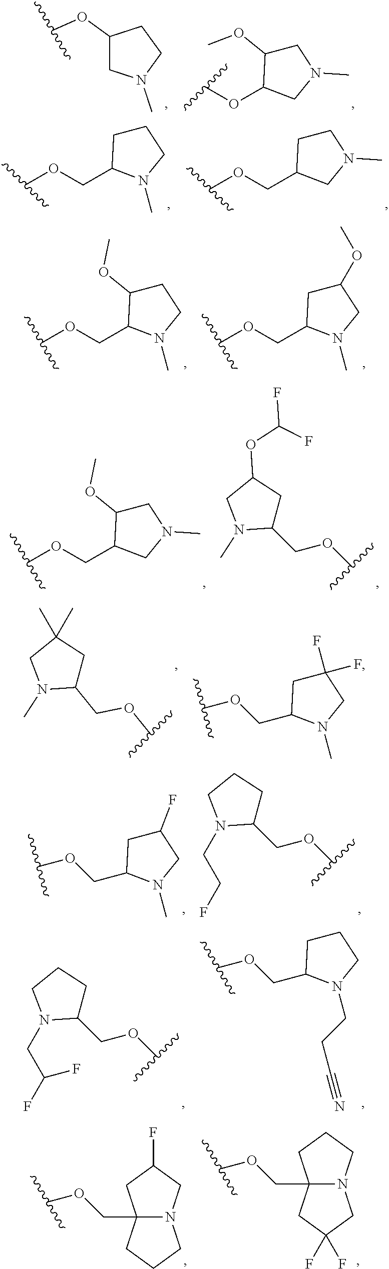

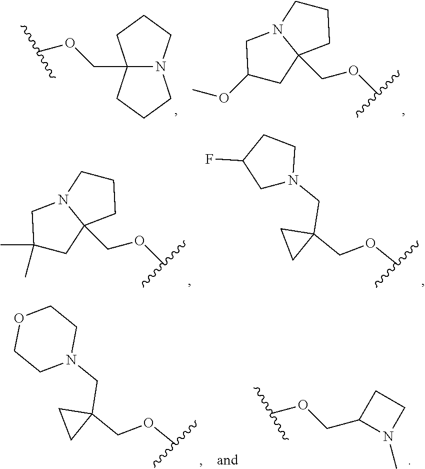

Embodiment II-56. The compound of embodiment II-53 or II-54, wherein R¹ is selected from:

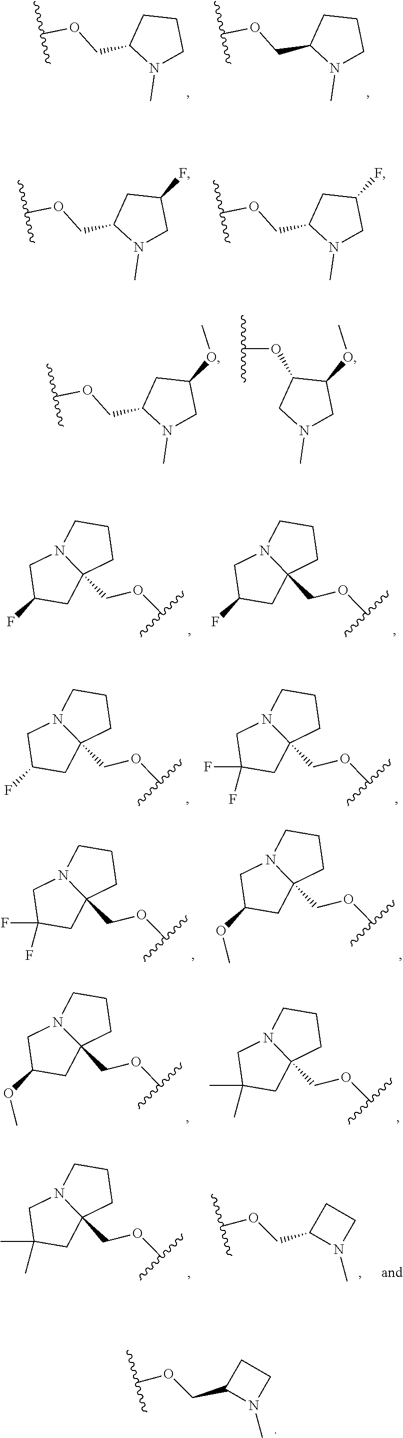

Embodiment II-57. The compound of any one of embodiments II-43 to II-51, wherein R¹ is a 4-6 membered heterocycle comprising a nitrogen atom that is unsubstituted or substituted with one or more R¹⁶.

Embodiment II-58. The compound of embodiment II-57, wherein R¹ is selected from:

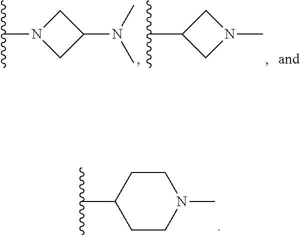

Embodiment II-59. The compound of any one of embodiments II-43 to II-58, wherein R⁶ is selected from:

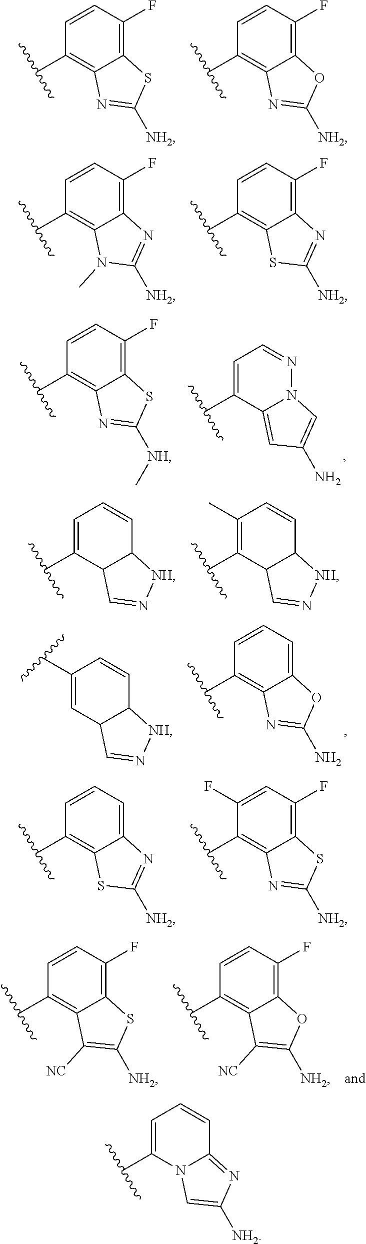

Embodiment II-60. The compound of embodiment II-59, wherein R⁶ is selected from:

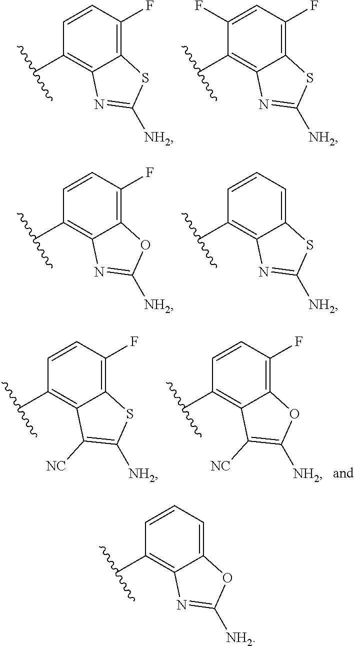

Embodiment II-61. The compound of embodiment II-60, wherein R⁶ is:

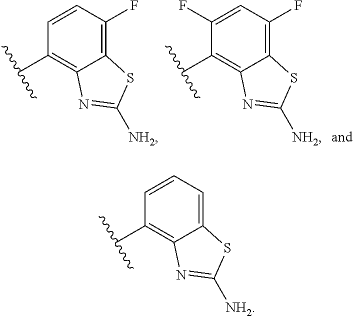 [SMILES: *c1ccc(F)c2sc(N)nc12 ; *c1c(F)cc(F)c2sc(N)nc12 ; *c1cccc2sc(N)nc12]

Embodiment II-62. The compound of any one of embodiments II-43 to II-61, wherein R⁴ is H.

Embodiment II-63. The compound of any one of embodiments II-43 to II-61, wherein R⁴ is a halogen.

Embodiment II-64. The compound of any one of embodiment II-43 to II-63, wherein R⁵ is a halogen.

Embodiment II-65. The compound of any one of embodiments II-43 to II-64, wherein R⁷ is a halogen.

Embodiment II-66. The compound of embodiment II-43, wherein (i) R³ is a 4- or 5-membered heterocycle including a single nitrogen atom, which heterocycle is unsubstituted or substituted with one or more R¹⁰; and (ii) R¹ is —OR⁸ where R⁸ is a heterocycle or alkylheterocycle, wherein the heterocycle of the heterocycle or alkylheterocycle is a 4-8-membered ring system comprising a single nitrogen atom.

Embodiment II-67. The compound of embodiment II-66, wherein R³ is selected from:

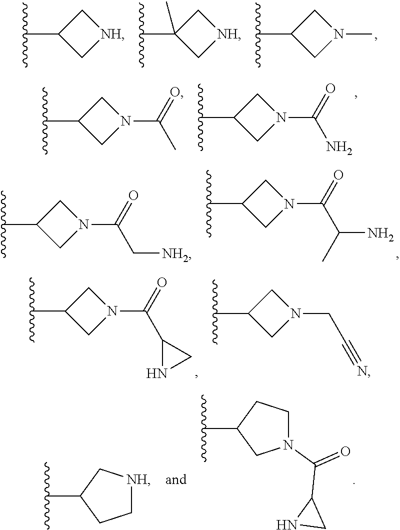

Embodiment II-68. The compound of embodiment II-66 or II-67, wherein (iii) R⁴ is H and/or (iv) R⁷ is a halogen.

Embodiment II-69. The compound of any one of embodiments II-66 to II-68 wherein R¹ is selected from:

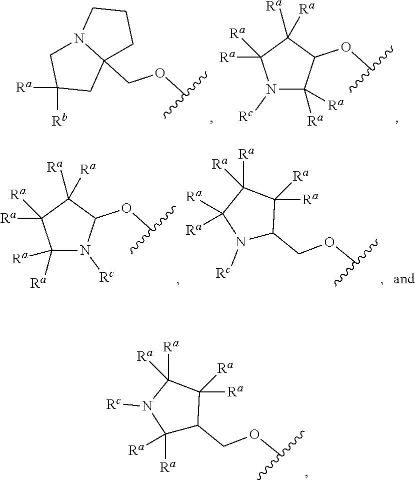

wherein each R^{a} and R^{b} are independently selected from halogen, C₁₋₆ alkyl, —OR¹², and H; and wherein R^{c} is selected from C₁₋₆ alkyl, wherein any C₁₋₆ alkyl of R^{a} or R^{c} is unsubstituted or is substituted with one or more R¹³.

Embodiment II-70. The compound of embodiment II-43, wherein (i) R³ is a bridged carbocyclic or heterocyclic ring system that is unsubstituted or substituted with one or more R¹⁰; and (ii) R¹ is —OR⁸ where R⁸ is a heterocycle or alkylheterocycle, wherein the heterocycle of the heterocycle or alkylheterocycle is a 4-8-membered ring system comprising a single nitrogen atom.

Embodiment II-71. The compound of embodiment II-71, wherein R³ is selected from:

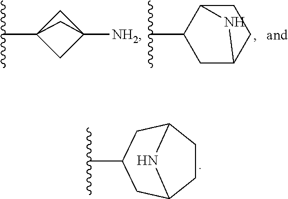

Embodiment II-72. The compound of embodiment II-70 or II-71, wherein (iii) R⁴ is H and/or (iv) R⁷ is a halogen.

Embodiment II-73. The compound of any one of embodiments II-70 to II-72, wherein R¹ is selected from:

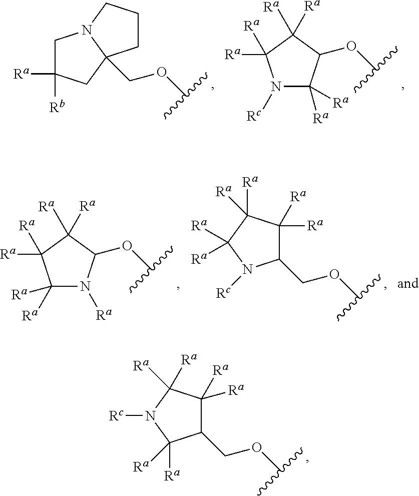

wherein each R^{a} and R^{b} are independently selected from halogen, C₁₋₆ alkyl, —OR¹², and H; and wherein R^{c} is selected from C₁₋₆ alkyl, wherein any C₁₋₆ alkyl of R^{a} or R^{c} is unsubstituted or is substituted with one or more R¹³.

Embodiment II-74. The compound of embodiment II-43, wherein (i) R³ is selected from C₁₋₆alkyl-N(R¹⁷)₂; and (ii) R¹ is —OR⁸ where R⁸ is a heterocycle or alkylheterocycle, wherein the heterocycle of the heterocycle or alkylheterocycle is a 4-8-membered ring system comprising a single nitrogen atom.

Embodiment II-75. The compound of embodiment II-74, wherein R³ is selected from:

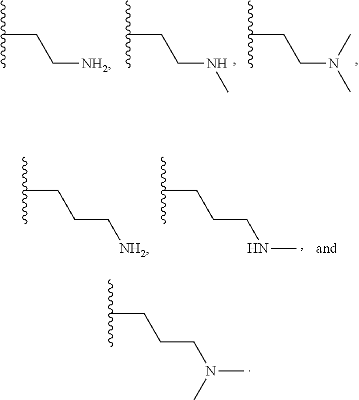

Embodiment II-76. The compound of embodiment II-74 or II-75, wherein (iii) R⁴ is H and/or (iv) R⁷ is a halogen.

Embodiment II-77. The compound of any one of embodiments II-74 to II-76 wherein R¹ is selected from:

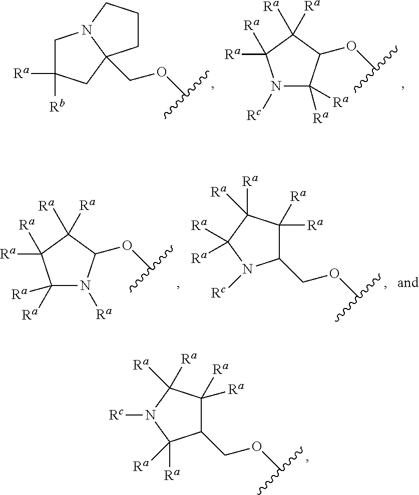

wherein each R^{a} and R^{b} are independently selected from halogen, C₁₋₆ alkyl, —OR¹², and H; and wherein R^{c} is selected from C₁₋₆ alkyl, wherein any C₁₋₆ alkyl of R^{a} or R^{c} is unsubstituted or is substituted with one or more R¹³.

Embodiment II-78. The compound of any one of embodiments II-66 to II-77, wherein R⁶ is:

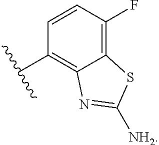 [SMILES: *c1ccc(F)c2sc(N)nc12]

Embodiment II-79. A compound according to Formula IA:

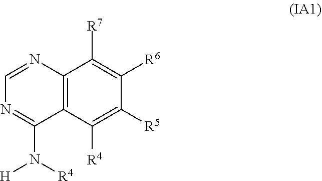

or a salt (e.g., pharmaceutically acceptable salt), ester, tautomer, prodrug, zwitterionic form, or stereoisomer thereof, wherein:
R¹ is H;
R² is H;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-80. The compound of embodiment II-79, wherein R³ is selected from a carbocycle and a heterocycle, wherein the carbocycle or heterocycle is unsubstituted or substituted with one or more R¹⁰.

Embodiment II-81. The compound of embodiment II-80, wherein R³ is selected from:

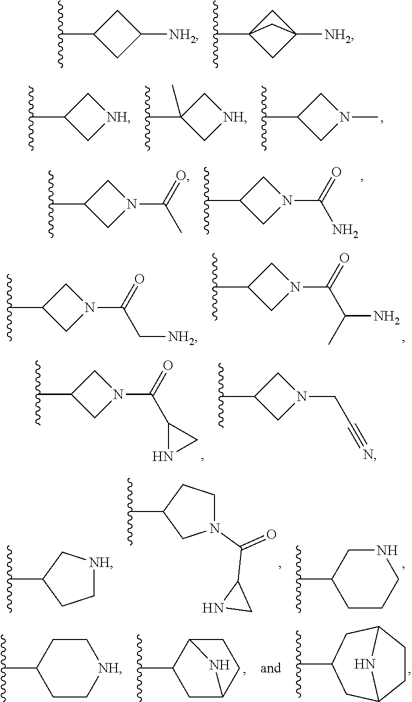

any of which is unsubstituted or substituted with one or more R¹⁰.

Embodiment II-82. The compound of embodiment II-79, wherein R³ is selected from C₁₋₆ alkyl that is substituted with one or more R⁹.

Embodiment II-83. The compound of embodiment II-82, wherein R³ is selected from:

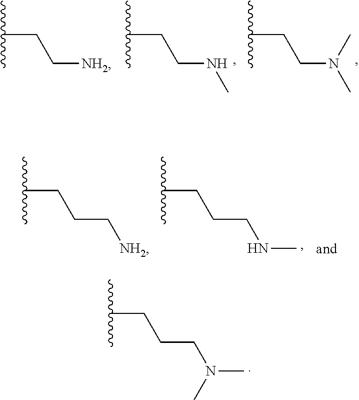

Embodiment II-84. The compound of embodiment II-82, wherein R³ is selected from C₁₋₆alkyl-N(R¹⁷)C(O)C₁₋₆alkylN(R¹⁷)₂.

Embodiment II-85. The compound of any one of embodiments II-79 to II-84, wherein R³ comprises an amino moiety.

Embodiment II-86. The compound of any one of embodiments II-79 to II-85, wherein R⁶ is selected from:

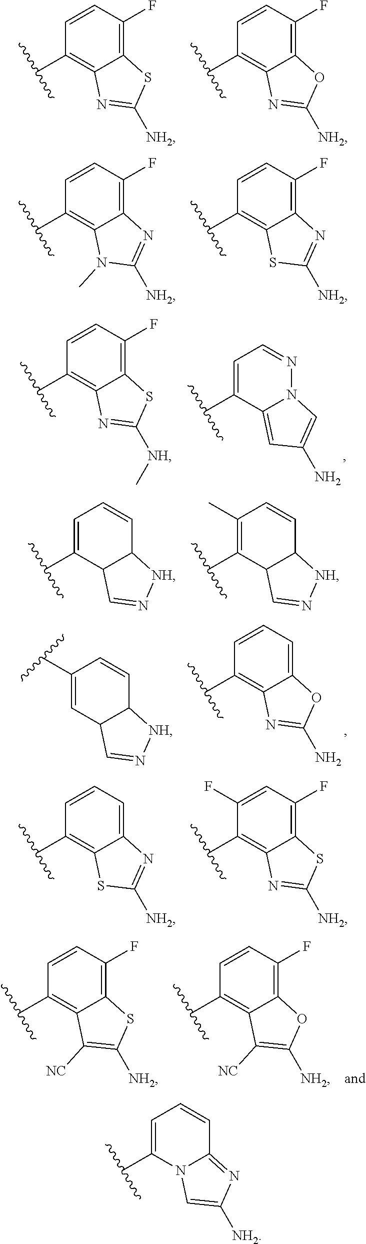

Embodiment II-87. The compound of embodiment II-86, wherein R⁶ is selected from:

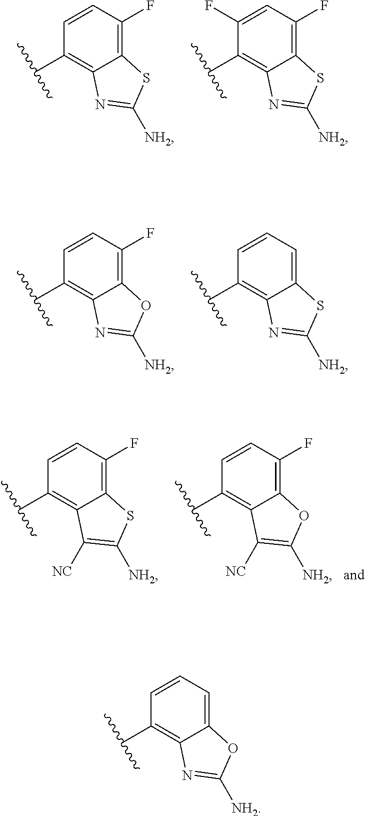

Embodiment II-88. The compound of embodiment II-87, wherein R⁶ is:

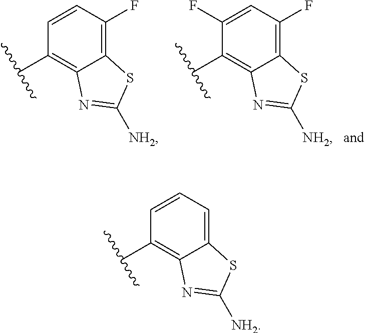 [SMILES: *c1ccc(F)c2sc(N)nc12 ; *c1c(F)cc(F)c2sc(N)nc12 ; *c1cccc2sc(N)nc12]

Embodiment II-89. The compound of any one of embodiments II-79 to II-88, wherein R⁴ is H.

Embodiment II-90. The compound of any one of embodiments II-79 to II-88, wherein R⁴ is a halogen.

Embodiment II-91. The compound of any one of embodiments II-79 to II-90, wherein R⁵ is a halogen.

Embodiment II-92. The compound of any one of embodiments II-79 to II-91, wherein R⁷ is a halogen.

Embodiment II-93. A compound according to Formula IA2:

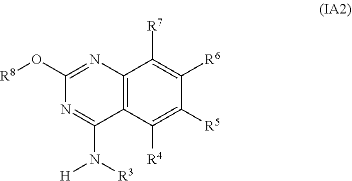

or a salt (e.g., pharmaceutically acceptable salt), ester, tautomer, prodrug, zwitterionic form, or stereoisomer thereof, wherein:
R¹ is —OR⁸;
R² is H;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-94. The compound of embodiment II-93, wherein R⁸ comprises a 4-8 membered heterocycle that is unsubstituted or substituted with one or more R¹⁶.

Embodiment II-95. The compound of embodiment II-93, wherein R¹ is selected from:

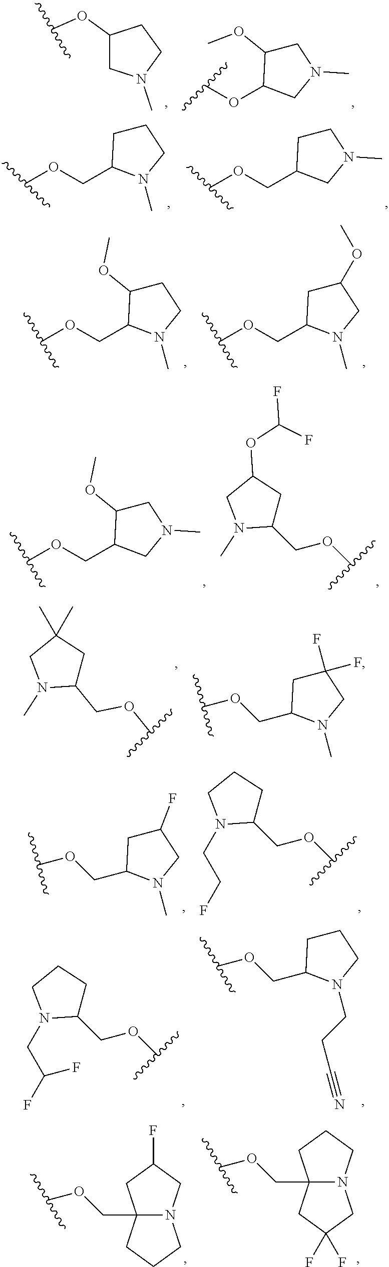

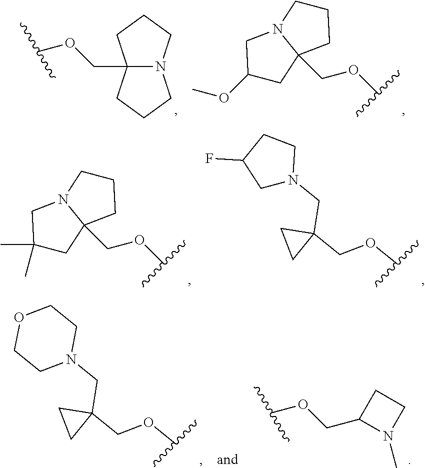

Embodiment II-96. The compound of embodiment II-93, wherein R¹ is selected from:

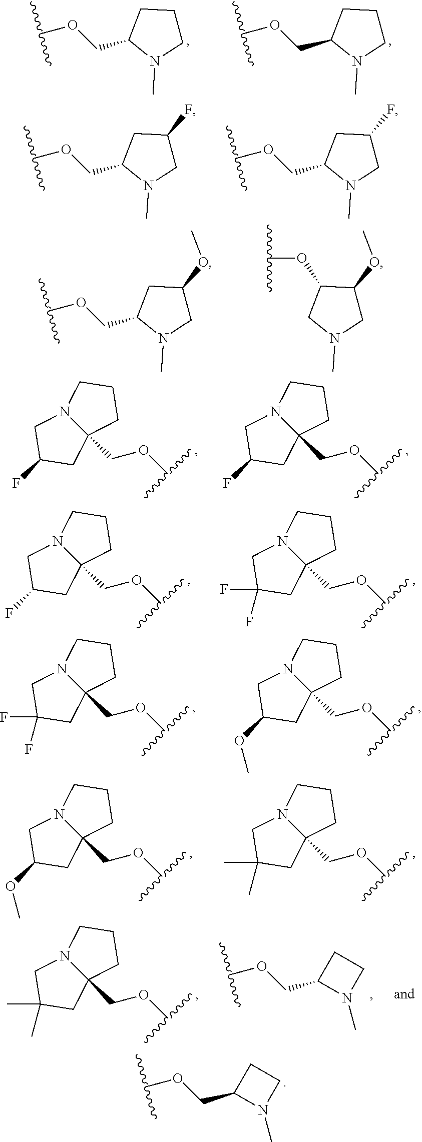

Embodiment II-97. The compound of any one of embodiments II-93 to II-96, wherein R³ is selected from a carbocycle and a heterocycle, wherein the carbocycle or heterocycle is unsubstituted or substituted with one or more R¹⁰.

Embodiment II-98. The compound of embodiment II-97, wherein R³ is selected from:

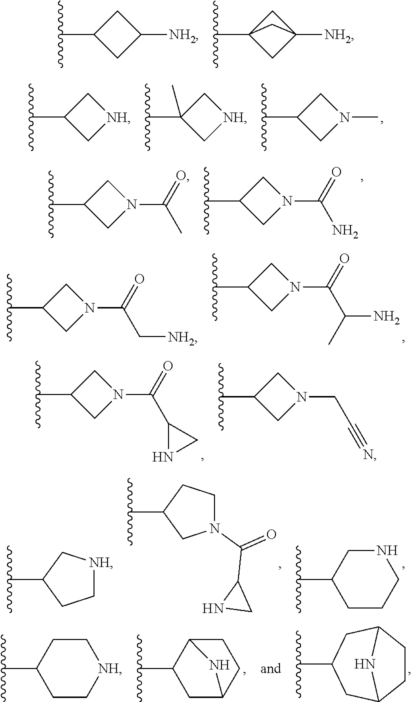

any of which is unsubstituted or substituted with one or more R¹⁰.

Embodiment II-99. The compound of any one of embodiments II-93 to II-96, wherein R³ is selected from C₁₋₆ alkyl that is substituted with one or more R⁹.

Embodiment II-100. The compound of embodiment II-99, wherein R³ is selected from:

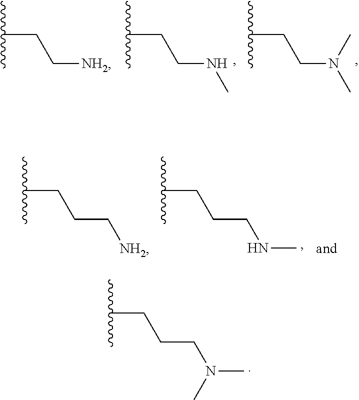

Embodiment II-101. The compound of any one of embodiments II-93 to II-96, wherein R³ is selected from C₁₋₆alkyl-N(R¹⁷)C(O)C₁₋₆alkylN(R¹⁷)₂.

Embodiment II-102. The compound of any one of embodiments II-93 to II-101, wherein R³ comprises an amino moiety.

Embodiment II-103. The compound of any one of embodiments II-93 to II-102, wherein R⁶ is selected from:

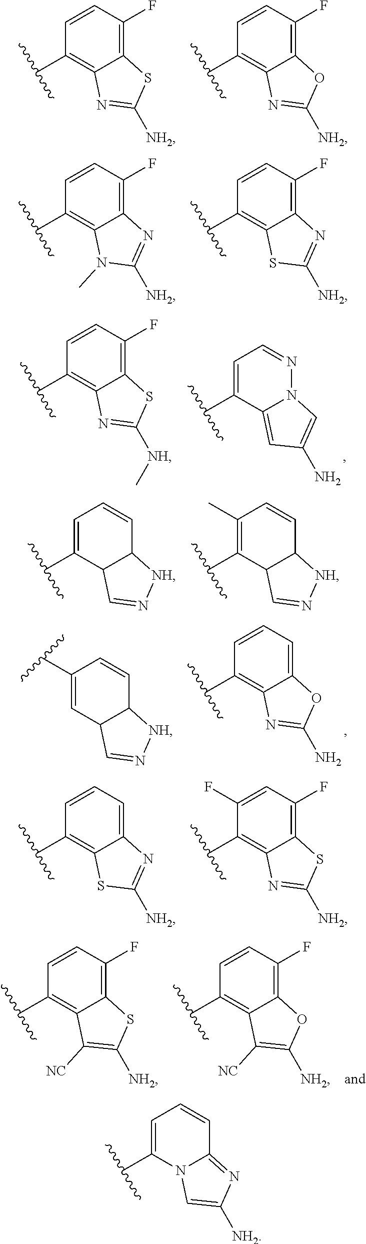

Embodiment II-104. The compound of embodiment II-103, wherein R⁶ is selected from:

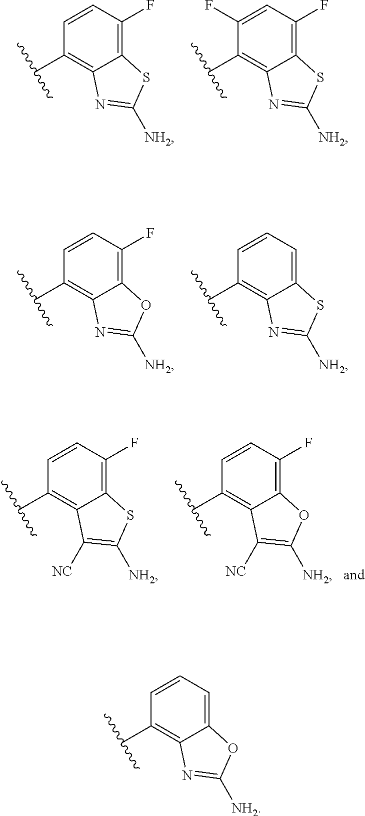

Embodiment II-105. The compound of embodiment II-104, wherein R⁶ is:

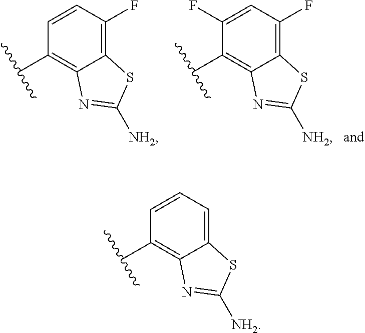 [SMILES: *c1ccc(F)c2sc(N)nc12 ; *c1c(F)cc(F)c2sc(N)nc12 ; *c1cccc2sc(N)nc12]

Embodiment II-106. The compound of any one of embodiments II-93 to II-105, wherein R⁴ is H.

Embodiment II-107. The compound of any one of embodiments II-93 to II-105, wherein R⁴ is a halogen.

Embodiment II-108. The compound of any one of embodiments II-93 to II-107, wherein R⁵ is a halogen.

Embodiment II-109. The compound of any one of embodiments II-93 to II-108, wherein R⁷ is a halogen.

Embodiment II-110. A compound according to Formula IB:

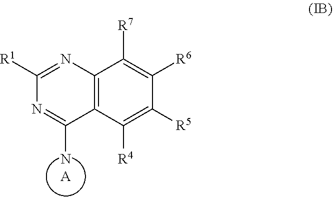

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
Ring A is a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-111. The compound of embodiment II-110, wherein Ring A is a 4-6 membered heterocycle that is unsubstituted or substituted with one or more R¹¹.

Embodiment II-112. The compound of embodiment II-110, wherein Ring A has the structure:

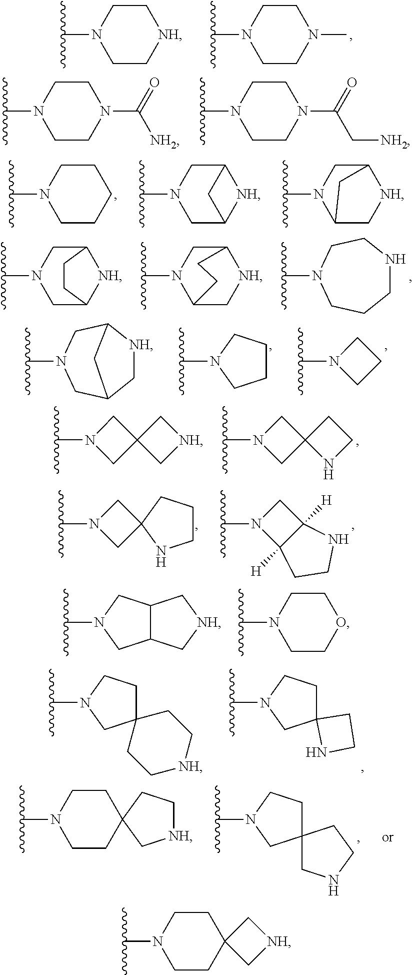

which ring is unsubstituted or substituted with one or more R¹¹.

Embodiment II-113. The compound of embodiment II-110, wherein Ring A has the structure:

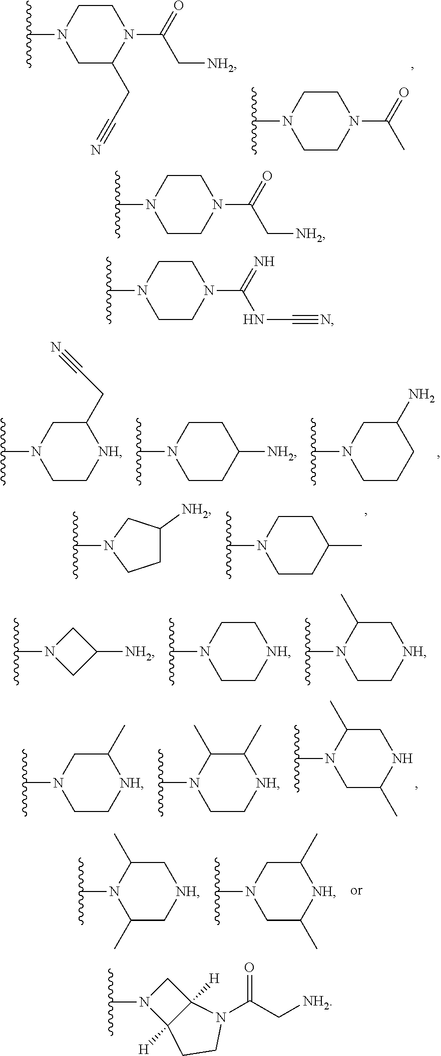

Embodiment II-114. The compound of any one of embodiments II-110 to II-113, wherein Ring A includes at least two nitrogen atoms and/or is substituted with a group including an amino moiety.

Embodiment II-115. The compound of any one of embodiments II-110 to II-114, wherein R¹ is H.

Embodiment II-116. The compound of any one of embodiments II-110 to II-114, wherein R¹ is —OR⁸.

Embodiment II-117. The compound of embodiment II-116, wherein R⁸ comprises a 3-8 membered heterocycle that is unsubstituted or substituted with one or more R¹⁶.

Embodiment II-118. The compound of embodiment II-117, wherein R¹ is selected from:

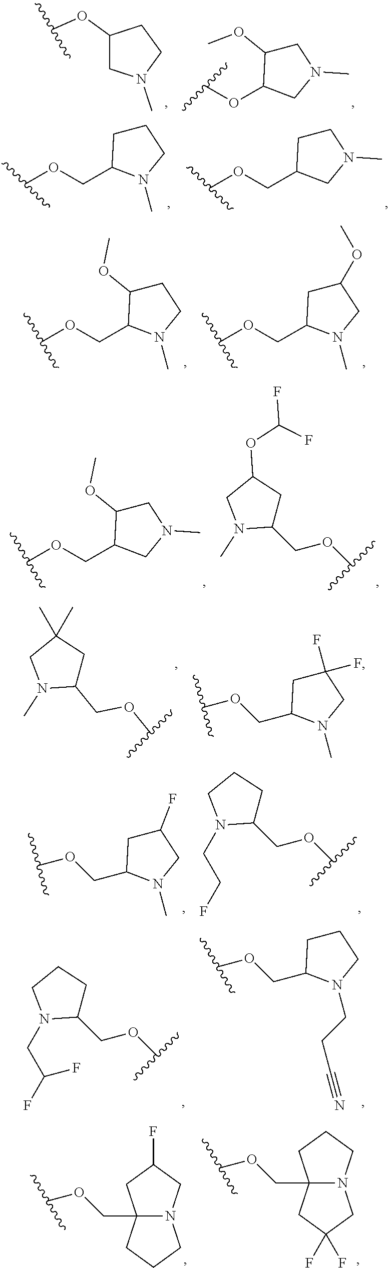

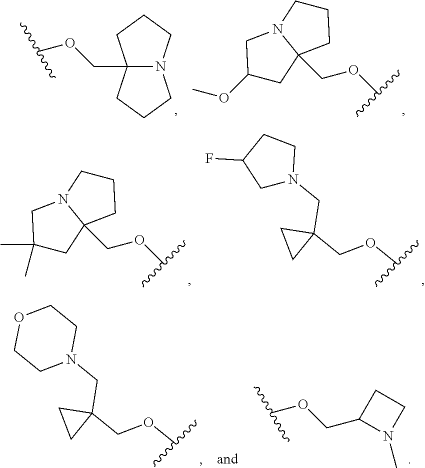

Embodiment II-119. The compound of embodiment II-117, wherein R¹ is selected from:

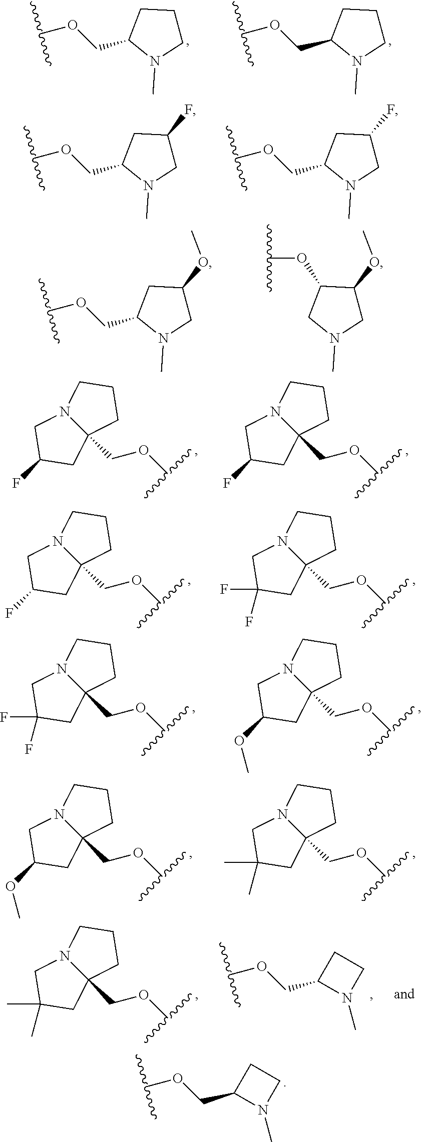

Embodiment II-120. The compound of any one of embodiments II-110 to II-114, wherein R¹ is a 4-6 membered heterocycle comprising a nitrogen atom that is unsubstituted or substituted with one or more R¹⁶.

Embodiment II-121. The compound of embodiment II-120, wherein R¹ is selected from:

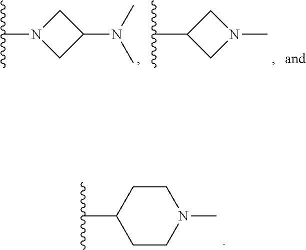

Embodiment II-122. The compound of any one of embodiments II-110 to II-121, wherein R⁶ is selected from:

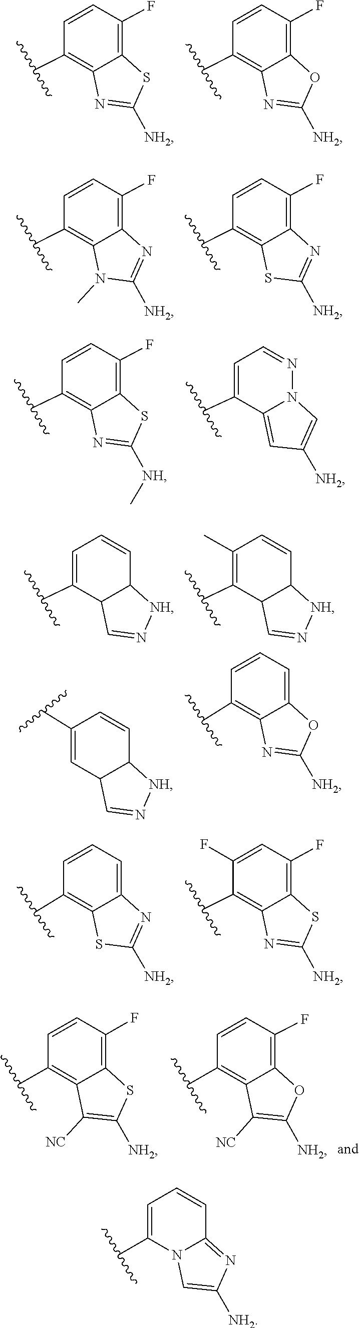

Embodiment II-123. The compound of embodiment II-122, wherein R⁶ is selected from:

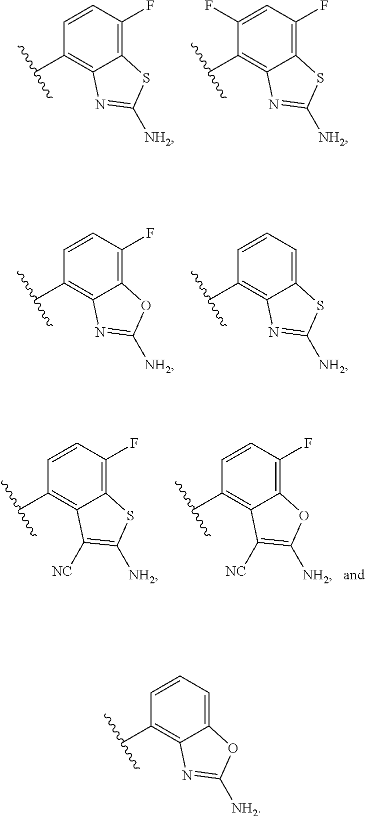

Embodiment II-124. The compound of embodiment II-123, wherein R⁶ is:

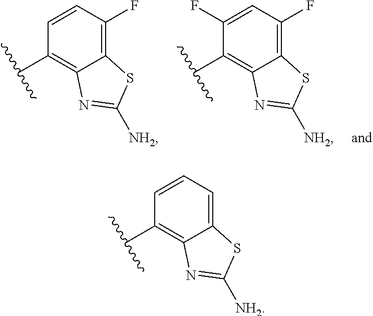 [SMILES: *c1ccc(F)c2sc(N)nc12 ; *c1c(F)cc(F)c2sc(N)nc12 ; "and" ; *c1cccc2sc(N)nc12]

Embodiment II-125. The compound of any one of embodiments II-110 to II-124, wherein R⁴ is H.

Embodiment II-126. The compound of any one of embodiments II-110 to II-124, wherein R⁴ is a halogen.

Embodiment II-127. The compound of any one of embodiments II-110 to II-126, wherein R⁵ is a halogen.

Embodiment II-128. The compound of any one of embodiments II-110 to II-127, wherein R⁷ is a halogen.

Embodiment II-129. A compound according to Formula IB1:

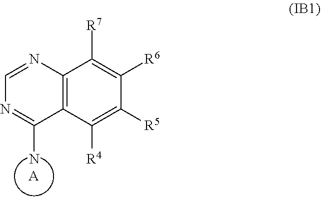

or a salt (e.g., pharmaceutically acceptable salt), ester, tautomer, prodrug, zwitterionic form, or stereoisomer thereof, wherein:
Ring A is a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-130. The compound of embodiment II-129, wherein Ring A is a 4-6 membered heterocycle that is unsubstituted or substituted with one or more R¹¹.

Embodiment II-131. The compound of embodiment II-129, wherein Ring A has the structure:

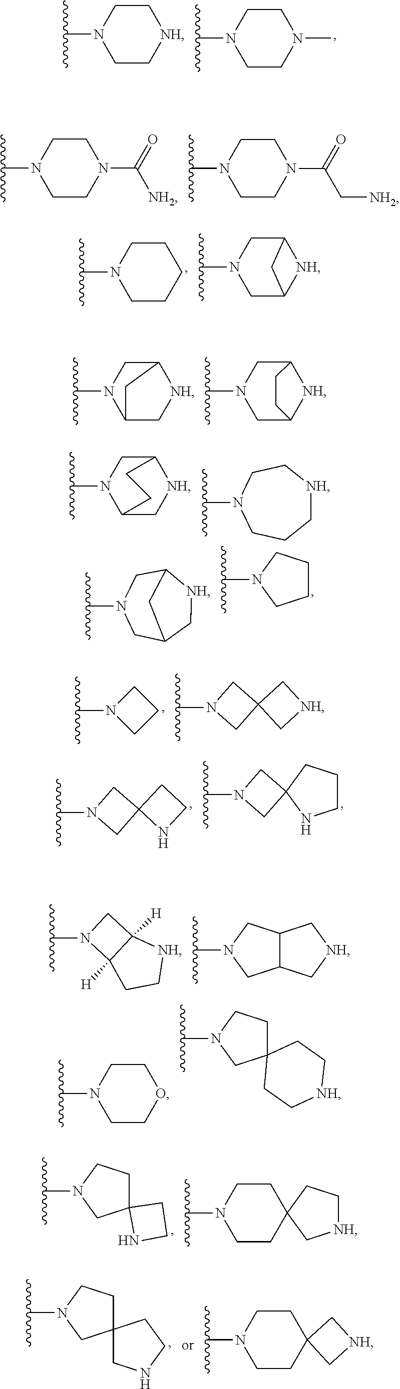

which ring is unsubstituted or substituted with one or more R¹¹.

Embodiment II-132. The compound of embodiment II-114, wherein Ring A has the structure:

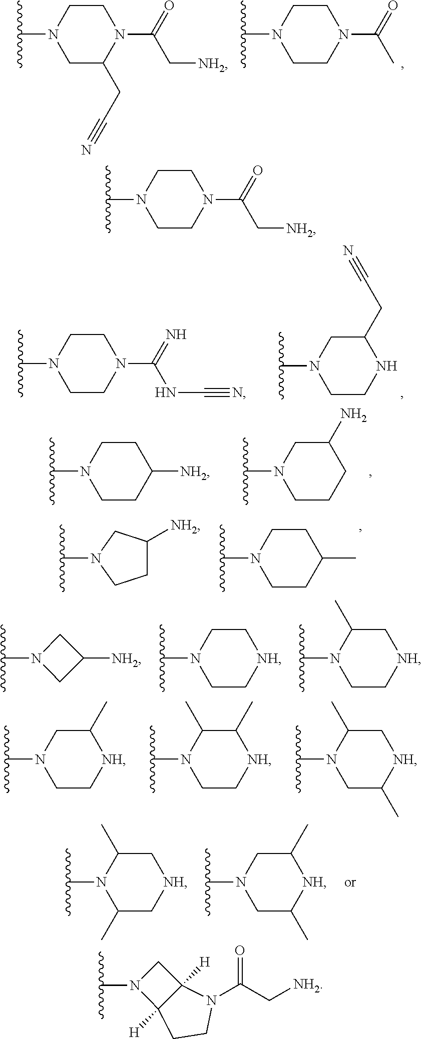

Embodiment II-133. The compound of any one of embodiments II-129 to II-132, wherein Ring A includes at least two nitrogen atoms and/or is substituted with a group including an amino moiety.

Embodiment II-134. The compound of any one of embodiments II-129 to II-133, wherein R⁶ is selected from:

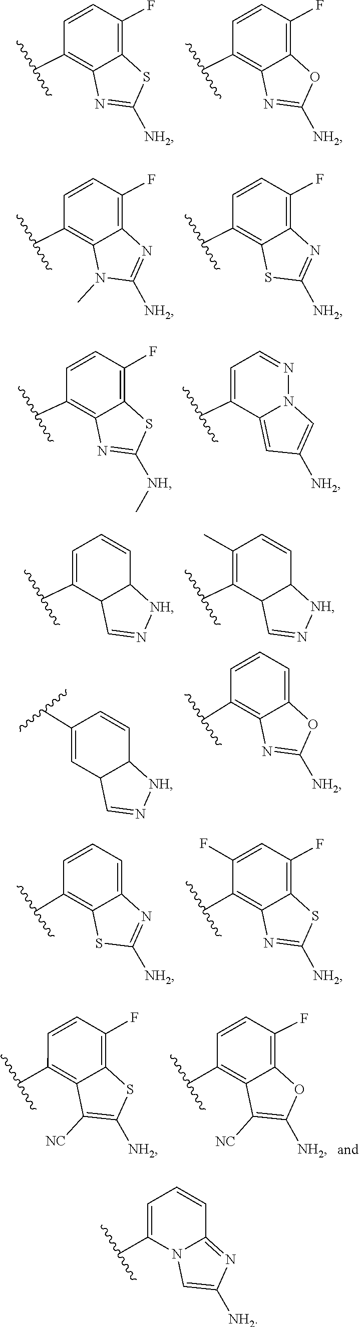

Embodiment II-135. The compound of embodiment II-134, wherein R⁶ is selected from:

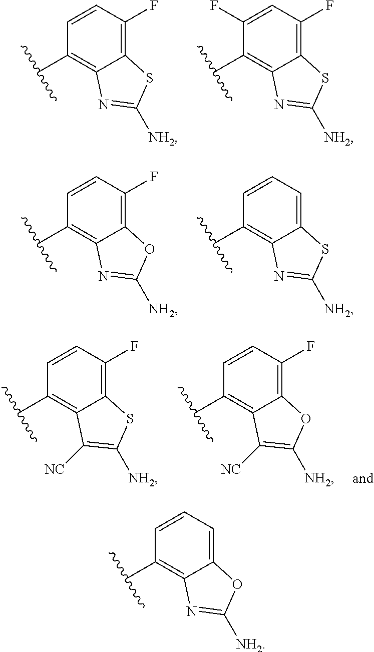

Embodiment II-136. The compound of embodiment II-135, wherein R⁶ is:

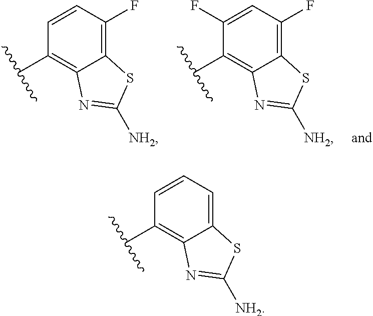 [SMILES: *c1ccc(F)c2sc(N)nc12 ; *c1c(F)cc(F)c2sc(N)nc12 ; "and" ; *c1cccc2sc(N)nc12]

Embodiment II-137. The compound of any one of embodiments II-129 to II-136, wherein R⁴ is H.

Embodiment II-138. The compound of any one of embodiments II-129 to II-136, wherein R⁴ is a halogen.

Embodiment II-139. The compound of any one of embodiments II-129 to II-138, wherein R⁵ is a halogen.

Embodiment II-140. The compound of any one of embodiments II-129 to II-139, wherein R⁷ is a halogen.

Embodiment II-141. The compound of embodiment II-129, wherein Ring A is a piperazine or diazepane that is unsubstituted or is substituted with one or more R¹¹.

Embodiment II-142. The compound of embodiment II-141, wherein Ring A is selected from:

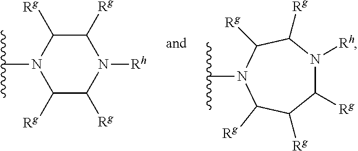

wherein each R⁹ is independently selected from H and C₁₋₆alkyl and R^{h} is selected from H, C₁₋₆alkyl, —C(O)NH₂, and —C(O)C₁₋₆alkylNH₂.

Embodiment II-143. The compound of embodiment II-142, wherein Ring A is selected from:

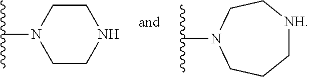 [SMILES: *N1CCNCC1 ; "and" ; *N1CCCNCC1]

Embodiment II-144. The compound of embodiment II-129, wherein Ring A is a bridged heterocyclic ring system that is unsubstituted or is substituted with one or more R¹¹.

Embodiment II-145. The compound of embodiment II-144, wherein Ring A is selected from:

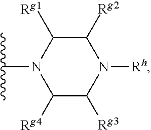

wherein (a) R^{g1} and R^{g3} or (b) R^{g2} and R^{g3} join together to form a second ring containing 4-6 members, and where any of R^{g1}, R^{g2}, R^{g3}, and R^{g4} that are not part of the second ring are independently selected from H and C₁₋₆alkyl; and wherein R^{h} is selected from H, C₁₋₆alkyl, —C(O)NH₂, and —C(O)C₁₋₆alkylNH₂.

Embodiment II-146. The compound of embodiment II-144, wherein Ring A is selected from:

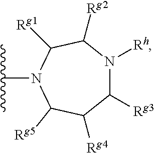

wherein R^{g2} and R^{g4} join together to form a second ring containing 4-6 members; R^{g1}, R^{g3}, and R^{g5} are independently selected from H and C₁₋₆alkyl; and R^{h} is selected from H, C₁₋₆alkyl, —C(O)NH₂, and —C(O)C₁₋₆alkylNH₂.

Embodiment II-147. The compound of embodiment II-144, wherein Ring A is selected from:

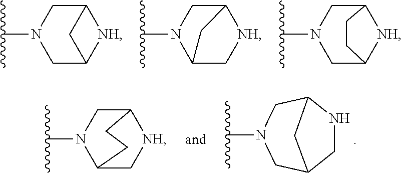

Embodiment II-148. The compound of any one of embodiments II-141 to II-147, wherein R⁶ is:

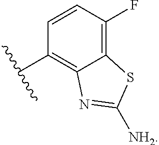 [SMILES: *c1ccc(F)c2sc(N)nc12]

Embodiment II-149. The compound of any one of embodiments II-141 to II-148, wherein R⁷ is a halogen and/or R⁴ is H.

Embodiment II-150. A compound according to Formula IB2:

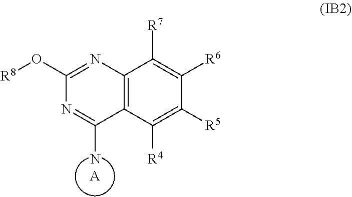

or a salt (e.g., pharmaceutically acceptable salt), ester, tautomer, prodrug, zwitterionic form, or stereoisomer thereof, wherein:
R¹ is —OR⁸;
Ring A is a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-151. The compound of embodiment II-150, wherein Ring A is a 4-6 membered heterocycle that is unsubstituted or substituted with one or more R¹¹.

Embodiment II-152. The compound of embodiment II-150, wherein Ring A has the structure:

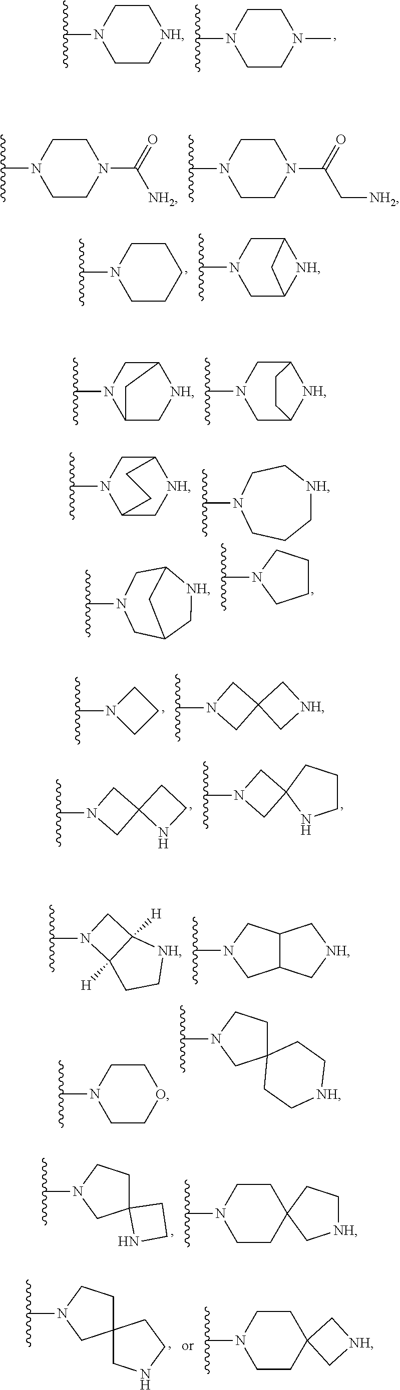

which ring is unsubstituted or substituted with one or more R¹¹.

Embodiment II-153. The compound of embodiment II-150, wherein Ring A has the structure:

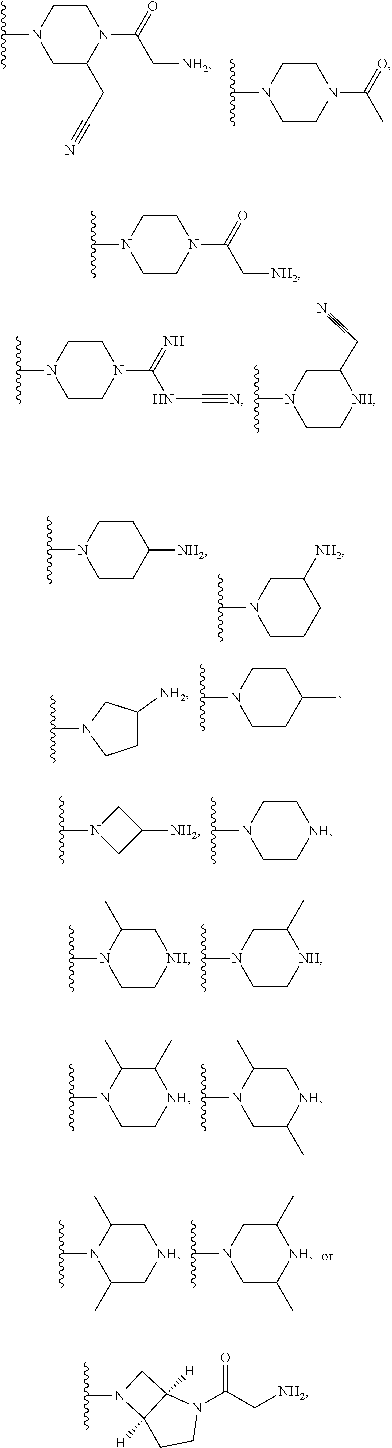

Embodiment II-154. The compound of any one of embodiments II-150 to II-153, wherein Ring A includes at least two nitrogen atoms and/or is substituted with a group including an amino moiety.

Embodiment II-155. The compound of any one of embodiments II-150 to II-154, wherein R⁸ comprises a 3-8 membered heterocycle that is unsubstituted or substituted with one or more R¹⁶.

Embodiment II-156. The compound of any one of embodiments II-150 to II-154, wherein R¹ is selected from:

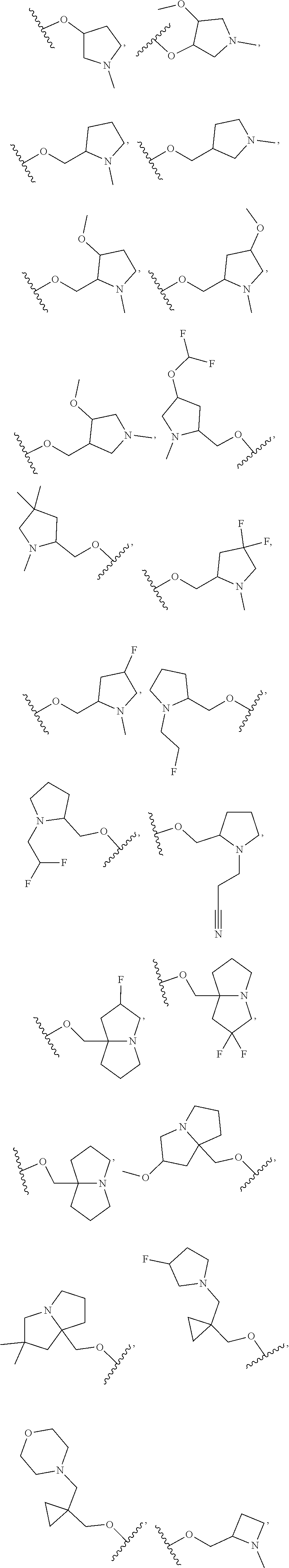

Embodiment II-157. The compound ofany one of embodiments II-150 to I-154, wherein R¹ is selected from:

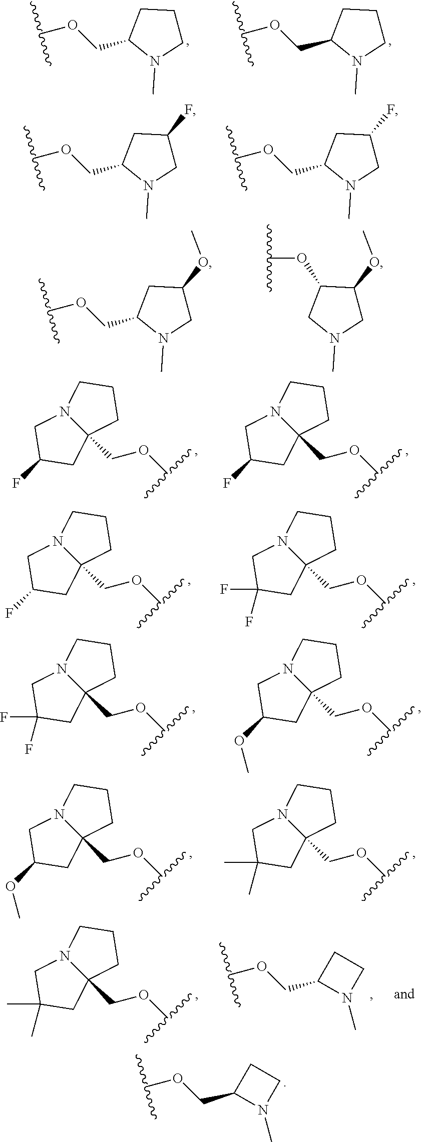

Embodiment II-158. The compound of any one of embodiments II-150 to II-157, wherein R⁶ is selected from:

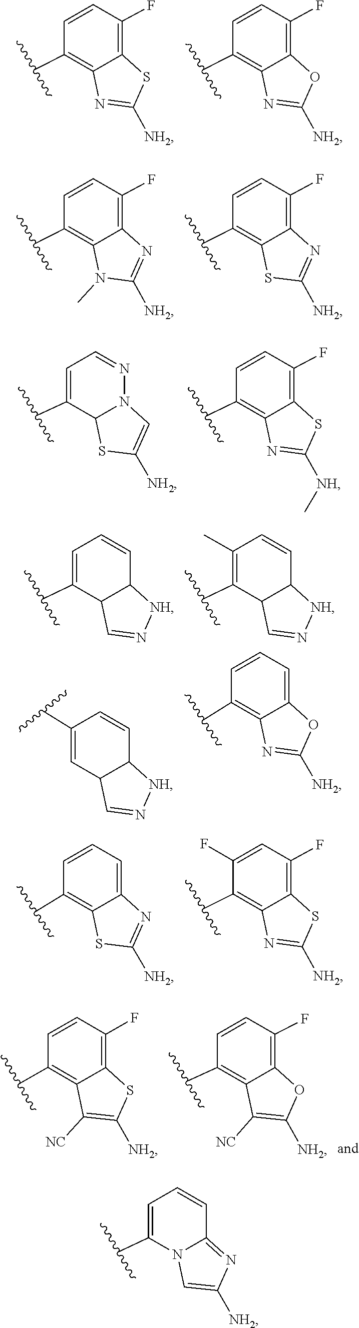

Embodiment II-159. The compound of embodiment II-158, wherein R⁶ is selected from:

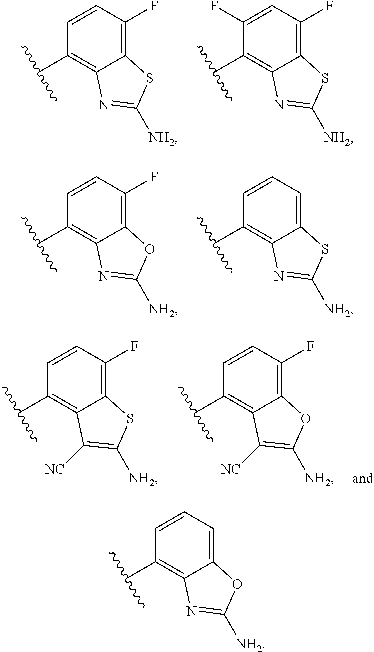

Embodiment II-160. The compound of embodiment II-159, wherein R⁶ is:

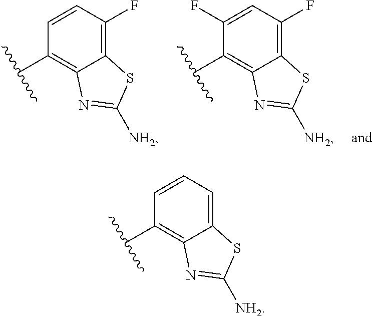 [SMILES: *c1ccc(F)c2sc(N)nc12 ; *c1c(F)cc(F)c2sc(N)nc12 ; "and" ; *c1cccc2sc(N)nc12]

Embodiment II-161. The compound of any one of embodiments II-150 to II-160, wherein R⁴ is H.

Embodiment II-162. The compound of any one of embodiments II-150 to II-160, wherein R⁴ is a halogen.

Embodiment II-163. The compound of any one of embodiments II-150 to II-162, wherein R⁵ is a halogen.

Embodiment II-164. The compound of any one of embodiments II-150 to II-163, wherein R⁷ is a halogen.

Embodiment II-165. The compound of embodiment II-150, wherein Ring A is selected from:

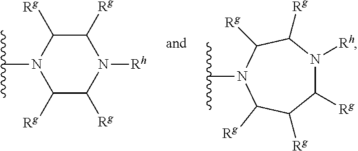

wherein each R⁹ is independently selected from H and C₁₋₆alkyl and R^{h} is selected from H, C₁₋₆alkyl, —C(O)NH₂, and —C(O)C₁₋₆alkylNH₂.

Embodiment II-166. The compound of embodiment II-165, wherein Ring A is selected from:

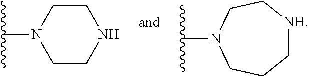 [SMILES: *N1CCNCC1 ; "and" ; *N1CCCNCC1]

Embodiment II-167. The compound of embodiment II-150, wherein Ring A is a bridged heterocyclic ring system that is unsubstituted or is substituted with one or more R¹¹.

Embodiment II-168. The compound of embodiment II-167, wherein Ring A is selected from:

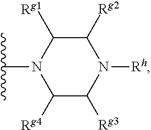

wherein (a) R^{g1} and R^{g3} or (b) R^{g2} and R^{g3} join together to form a second ring containing 4-6 members, and where any of R^{g1}, R^{g2}, R^{g3}, and R^{g4} that are not part of the second ring are independently selected from H and C₁₋₆alkyl; and wherein R^{h} is selected from H, C₁₋₆alkyl, —C(O)NH₂, and —C(O)C₁₋₆alkylNH₂.

Embodiment II-169. The compound of embodiment II-167, wherein Ring A is selected from:

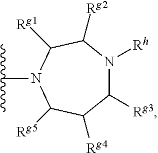

where R^{g2} and R^{g4} join together to form a second ring containing 4-6 members; R^{g1}, R^{g3}, and R^{g5} are independently selected from H and C₁₋₆alkyl; and R^{h} is selected from H, C₁₋₆alkyl, —C(O)NH₂, and —C(O)C₁₋₆alkylNH₂.

Embodiment II-170. The compound of embodiment II-167, wherein Ring A is selected from:

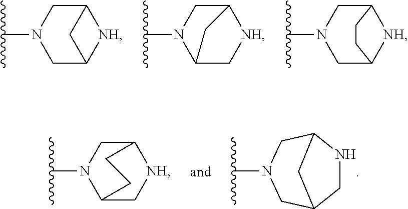

Embodiment II-171. The compound of any one of embodiments II-165 to II-170, wherein R⁶ is:

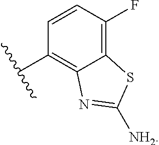 [SMILES: *c1ccc(F)c2sc(N)nc12]

Embodiment II-172. The compound of any one of embodiments II-165 to II-171, wherein R⁷ is a halogen and/or R⁴ is H.

Embodiment II-173. The compound of any one of embodiments II-165 to II-172, wherein R¹ is selected from:

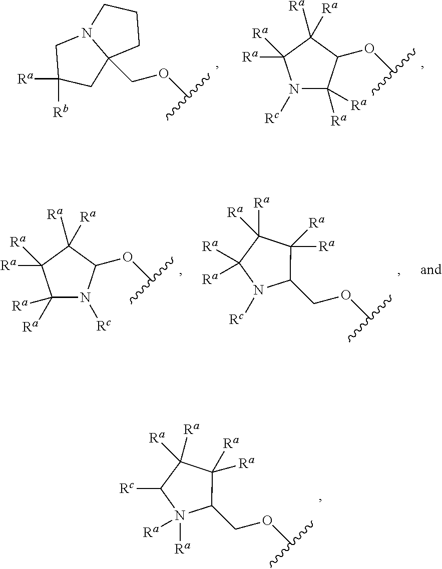

wherein each R^{a} and R^{b} are independently selected from halogen, C₁₋₆ alkyl, —OR¹², and H; and wherein R^{c} is selected from C₁₋₆ alkyl, wherein any C₁₋₆ alkyl of R^{a} and R^{c} is unsubstituted or is substituted with one or more R¹³.

Embodiment II-174. A compound according to Formula IC:

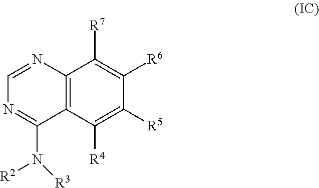

or a salt (e.g., pharmaceutically acceptable salt), ester, tautomer, prodrug, zwitterionic form, or stereoisomer thereof, wherein:
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-175. The compound of embodiment II-174, wherein R³ is selected from a carbocycle and a heterocycle, wherein the carbocycle or heterocycle is unsubstituted or substituted with one or more R¹⁰.

Embodiment II-176. The compound of embodiment II-174, wherein R³ is selected from:

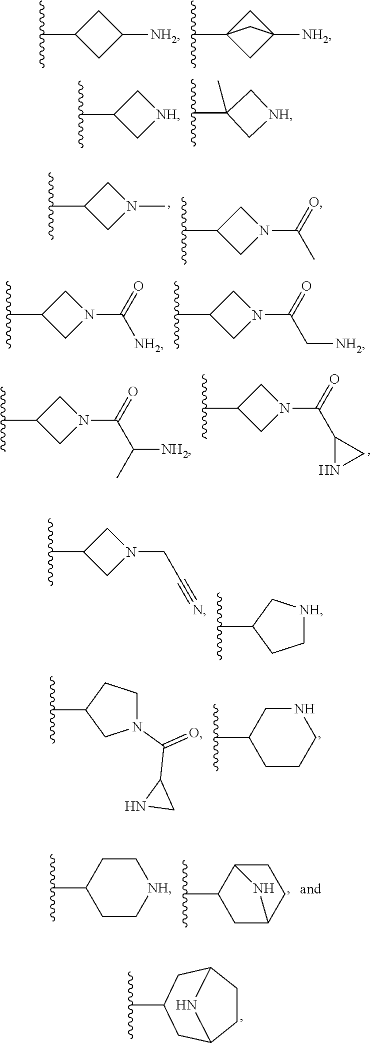

any of which is unsubstituted or substituted with one or more R¹⁰.

Embodiment II-177. The compound of embodiment II-174, wherein R³ is selected from C₁₋₆ alkyl that is substituted with one or more R⁹.

Embodiment II-178. The compound of embodiment II-177, wherein R³ is selected from:

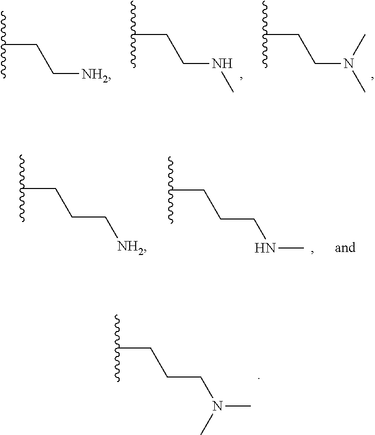

Embodiment II-179. The compound of embodiment II-174, wherein R³ is selected from C₁₋₆alkyl-N(R¹⁷)C(O)C₁₋₆alkylN(R¹⁷)₂.

Embodiment II-180. The compound of any one of embodiments II-174 to II-179, wherein R³ includes an amino moiety.

Embodiment II-181. The compound of embodiment II-174, wherein R² and R³, together with the atom to which they are attached, form a 4-6 membered heterocycle that is unsubstituted or substituted with one or more R¹¹.

Embodiment II-182. The compound of embodiment II-174, wherein R² and R³, together with the atom to which they are attached, form a heterocycle having the structure:

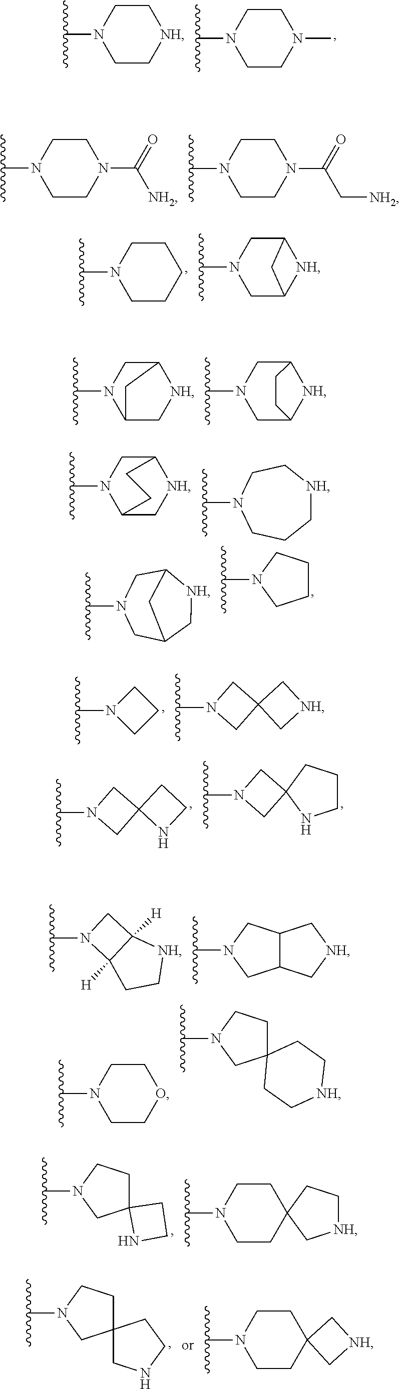

which ring is unsubstituted or substituted with one or more R¹¹.

Embodiment II-183. The compound of embodiment II-174, wherein R² and R³, together with the atom to which they are attached, form a heterocycle having the structure:

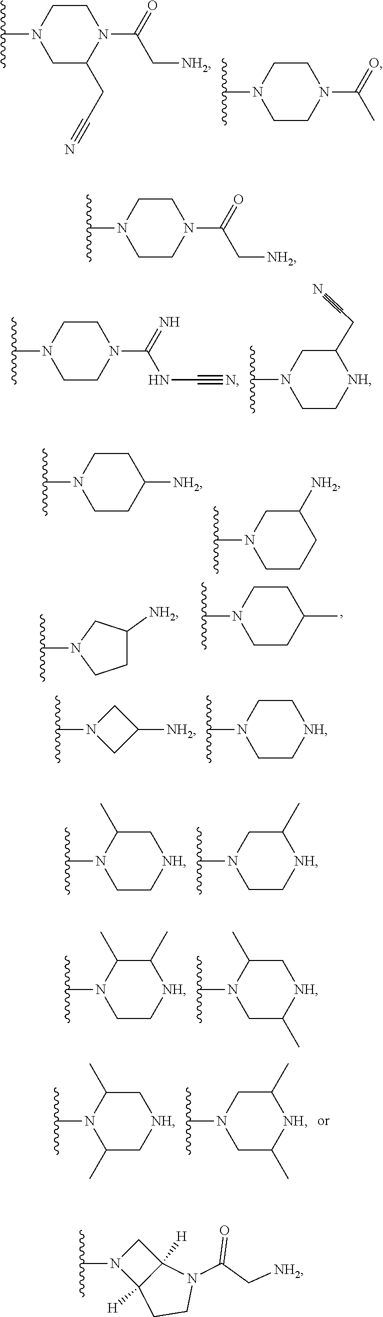

Embodiment II-184. The compound of any one of embodiments II-181 to II-183, wherein R² and R³, together with the atom to which they are attached, form a heterocycle that (i) comprises an additional nitrogen atom or (ii) is substituted with a group including an amino moiety.

Embodiment II-185. The compound of any one of embodiments II-174 to II-184, wherein R⁶ is selected from:

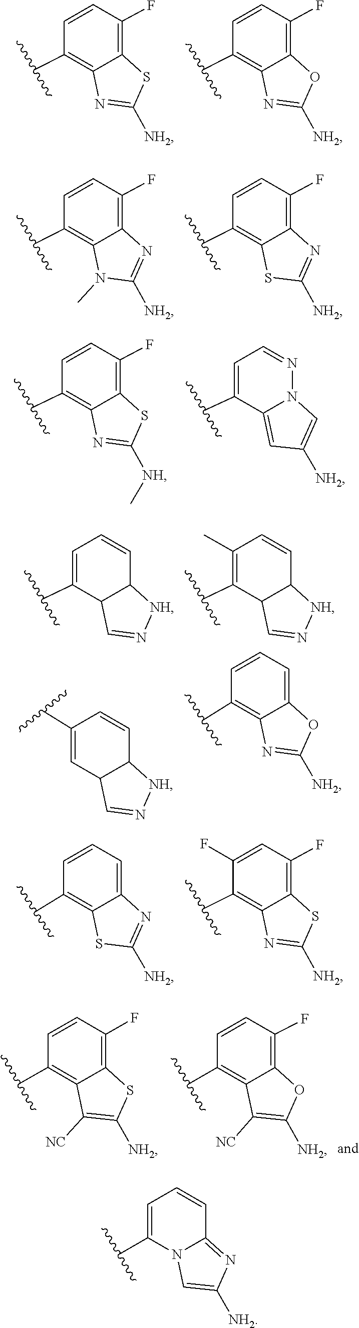

Embodiment II-186. The compound of embodiment II-185, wherein R⁶ is selected from:

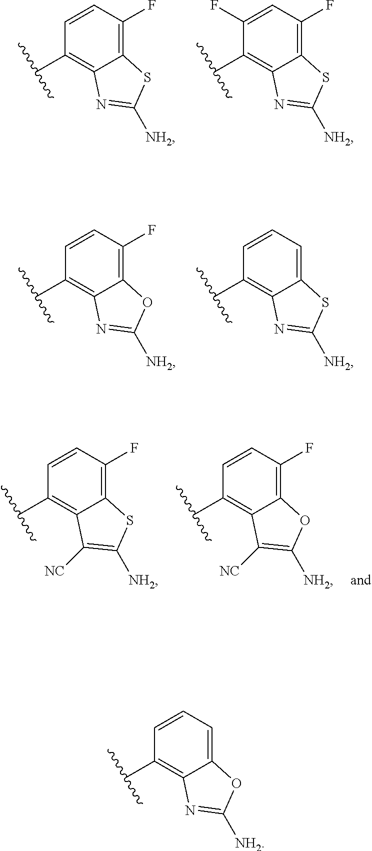

Embodiment II-187. The compound of embodiment II-186 wherein R⁶ is:

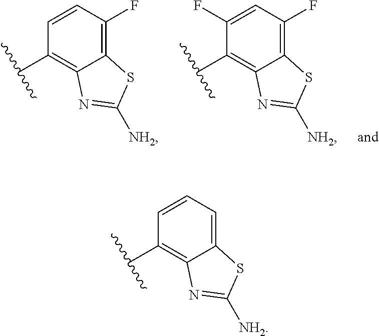 [SMILES: *c1ccc(F)c2sc(N)nc12 ; *c1c(F)cc(F)c2sc(N)nc12 ; *c1cccc2sc(N)nc12]

Embodiment II-188. The compound of anyone of embodiments II-174 to II-187, wherein R⁴ is H.

Embodiment II-189. The compound of any one of embodiments II-174 to II-187, wherein R⁴ is a halogen.

Embodiment II-190. The compound of any one of embodiments II-174 to II-189, wherein R⁵ is a halogen.

Embodiment II-191. The compound of any one of embodiments II-174 to II-190, wherein R⁷ is a halogen.

Embodiment II-192. A compound according to Formula ID:

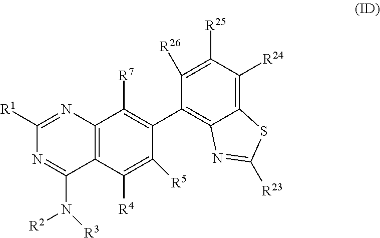

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰; each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H;
R²³ is selected from —N(R¹²)₂ and C₁₋₆alkyl-N(R¹²)₂;
R²⁴, R²⁵, and R²⁶ are independently selected from H, halogen, —OR¹², and C₁₋₆alkyl, wherein any C₁₋₆alkyl is unsubstituted or substituted with one or more R¹¹; and
each R²⁷ is independently selected from —OH, —OC₁₋₆alkyl, —CN, —NH₂, —NHC₁₋₆alkyl, and halogen.

Embodiment II-193. The compound of embodiment II-192, wherein R³ is selected from a carbocycle and a heterocycle, wherein the carbocycle or heterocycle is unsubstituted or substituted with one or more R¹⁰.

Embodiment II-194. The compound of embodiment II-193, wherein R³ is selected from:

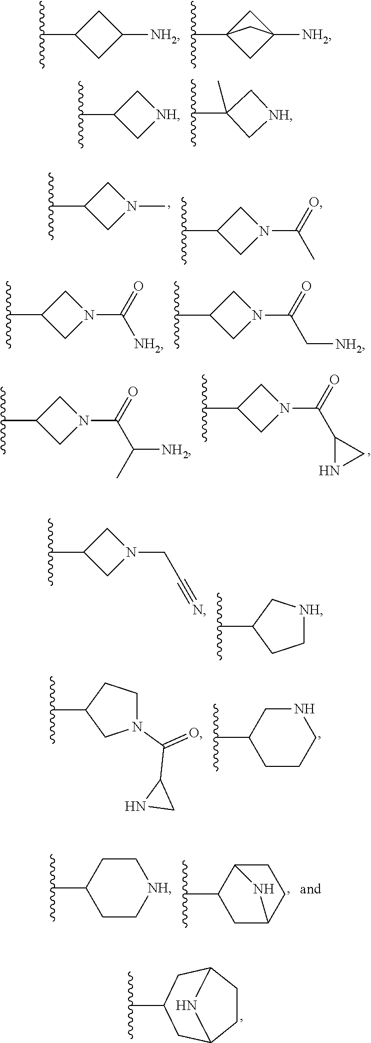

any of which is unsubstituted or substituted with one or more R¹⁰.

Embodiment II-195. The compound of embodiment II-192, wherein R³ is selected from C₁₋₆ alkyl that is substituted with one or more R⁹.

Embodiment II-196. The compound of embodiment II-195, wherein R³ is selected from:

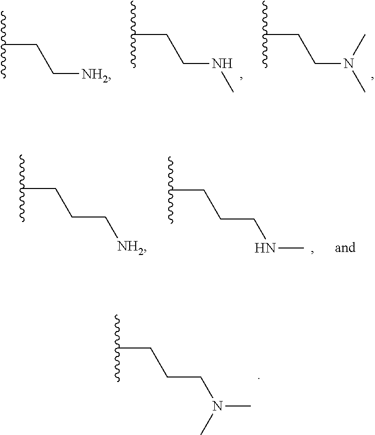

Embodiment II-197. The compound of embodiment II-192, wherein R³ is selected from C₁₋₆alkyl-N(R¹⁷)C(O)C₁₋₆alkylN(R¹⁷)₂.

Embodiment II-198. The compound of any one of embodiments II-192 to II-197, wherein R³ comprises an amino moiety.

Embodiment II-199. The compound of embodiment II-192, wherein R² and R³, together with the atom to which they are attached, form a 4-6 membered heterocycle that is unsubstituted or substituted with one or more R¹¹.

Embodiment II-200. The compound of embodiment II-199, wherein R² and R³, together with the atom to which they are attached, form a heterocycle having the structure:

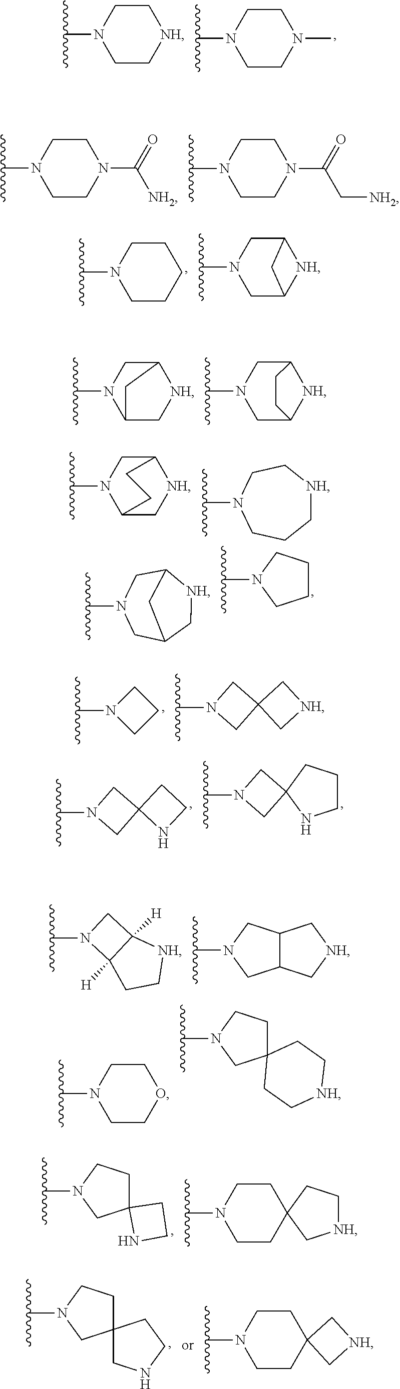

which ring is unsubstituted or substituted with one or more R¹¹.

Embodiment II-201. The compound of embodiment II-199, wherein R² and R³, together with the atom to which they are attached, form a heterocycle having the structure:

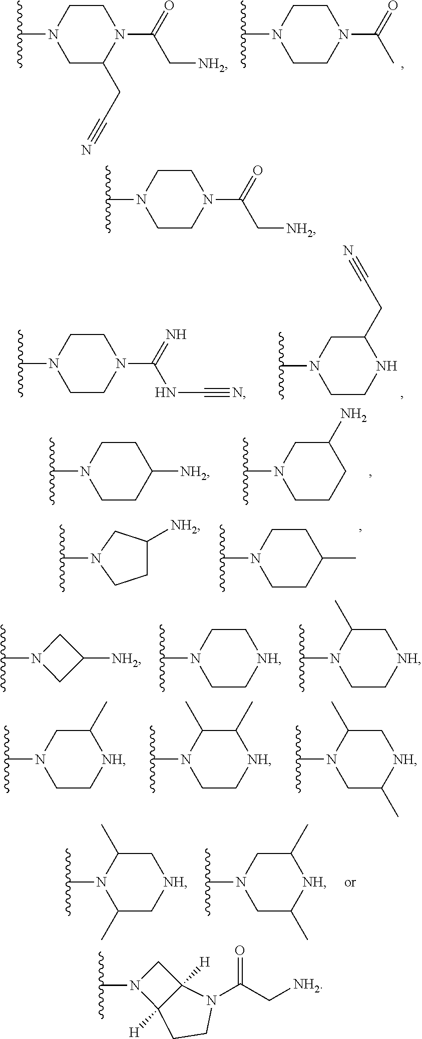

Embodiment II-202. The compound of any one of embodiments II-199 to II-201, wherein R² and R³, together with the atom to which they are attached, form a heterocycle that comprises (i) an additional nitrogen atom or (ii) is substituted with a group including an amino moiety.

Embodiment II-203. The compound of any one of embodiments II-192 to II-202, wherein R¹ is H.

Embodiment II-204. The compound of any one of embodiments II-192 to II-202, wherein R¹ is —OR⁸.

Embodiment II-205. The compound of embodiment II-204, wherein R⁸ comprises a 3-8 membered heterocycle that is unsubstituted or substituted with one or more R¹⁶.

Embodiment II-206. The compound of any one of embodiments II-192 to II-202, wherein R¹ is selected from:

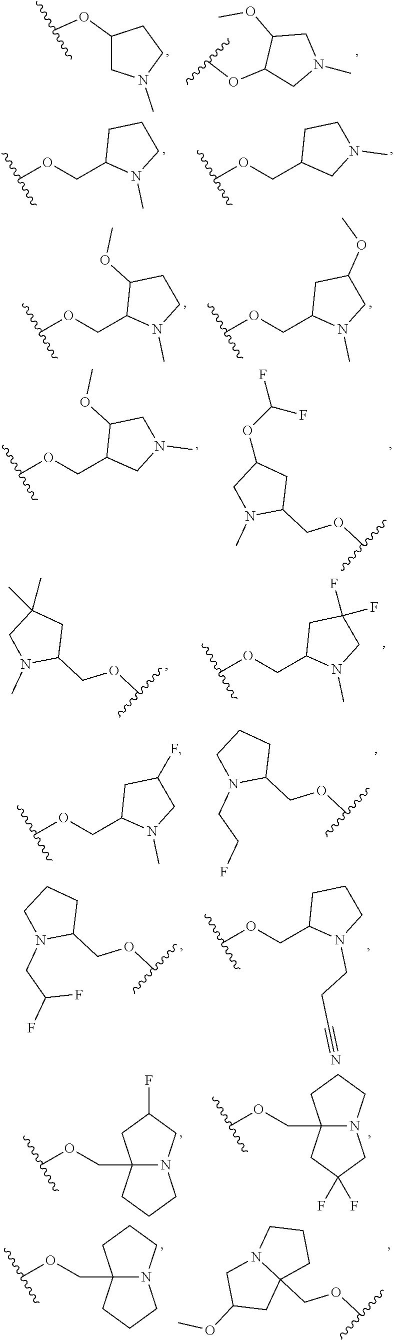

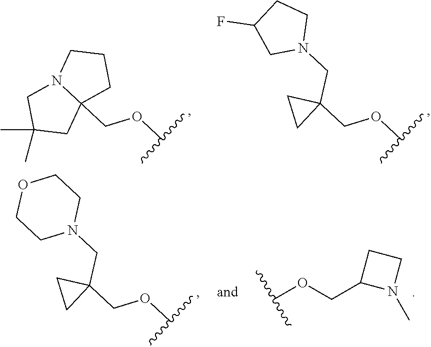

Embodiment II-207. The compound of any one of embodiments II-192 to II-202, wherein R¹ is selected from:

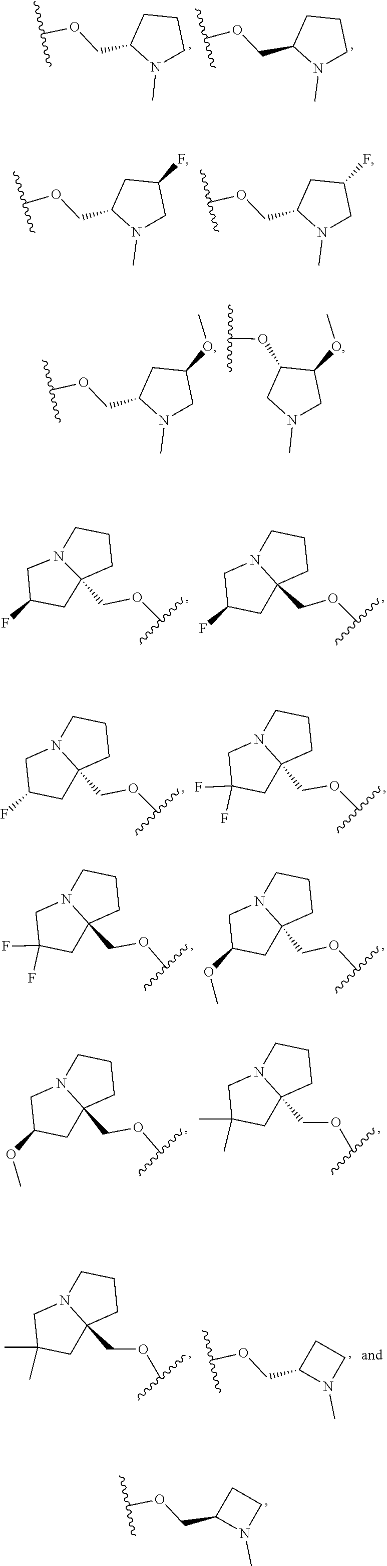

Embodiment II-208. The compound of any one of embodiments II-192 to II-202, wherein R¹ is a 4-6 membered heterocycle comprising a nitrogen atom that is unsubstituted or substituted with one or more R¹⁶.

Embodiment II-209. The compound of embodiment II-208, wherein R¹ is selected from:

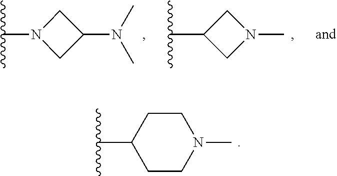

Embodiment II-210. The compound of any one of embodiments II-192 to II-209, wherein R⁴ is H.

Embodiment II-211. The compound of any one of embodiments II-192 to II-209, wherein R⁴ is a halogen.

Embodiment II-212. The compound of any one of embodiments II-192 to II-211, wherein R⁵ is a halogen.

Embodiment II-213. The compound of any one of embodiments II-192 to II-212, wherein R⁷ is a halogen.

Embodiment II-214. The compound of any one of embodiments II-192 to II-213, wherein R²³ is —N(R¹²)₂.

Embodiment II-215. The compound of embodiment II-214, wherein R²³ is —NH₂.

Embodiment II-216. The compound of any one of embodiments II-192 to II-215, wherein R²⁴ is a halogen.

Embodiment II-217. The compound of any one of embodiments II-192 to II-216, wherein R²⁵ and R²⁶ are H.

Embodiment II-218. A compound according to Formula IE:

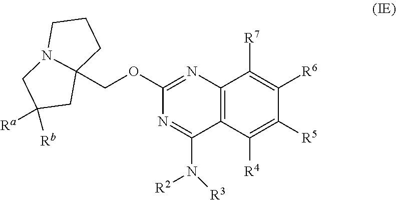

or a salt (e.g., pharmaceutically acceptable salt), ester, tautomer, prodrug, zwitterionic form, or stereoisomer thereof, wherein:
R² is selected from H, a 3-6 membered carbocycle, and C₁₋₆ alkyl, wherein the 3-6 membered carbocycle or the C₁₋₆ alkyl is unsubstituted or substituted with one or more R¹³;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic carbocycle or heterocycle substituted with one or more R¹⁵;
R⁷ is selected from halogen, —OR¹², —CN, and H;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen;
each R²² is independently selected from C₁₋₆ alkyl, C₂₋₆ alkenyl, and H; and
R^{a} and R^{b} are independently selected from halogen, —OR¹², C₁₋₆alkyl, and H, wherein any C₁₋₆alkyl is unsubstituted or is substituted with one or more R¹³.

Embodiment II-219. The compound of embodiment II-218, wherein R^{a} and/or R^{b} is a halogen.

Embodiment II-220. The compound of embodiment II-218 or II-219, wherein R^{a} and/or R^{b} is C₁₋₆alkyl or —OC₁₋₆alkyl, wherein any C₁₋₆alkyl is unsubstituted or is substituted with one or more R¹³.

Embodiment II-221. The compound of any one of embodiments II-218 to II-220, wherein R^{a} and/or R^{b} is H.

Embodiment II-222. The compound of any one of embodiments II-218 to II-221, wherein R² is H.

Embodiment II-223. The compound of any one of embodiments II-218 to II-221, wherein R² is selected from C₁₋₆ alkyl and a 3-6 membered carbocycle.

Embodiment II-224. The compound of any one of embodiments II-218 to II-222, wherein R³ is C₁₋₆ alkyl, which C₁₋₆alkyl is substituted with one or more R⁹.

Embodiment II-225. The compound of any one of embodiments II-218 to II-222, wherein R³ is a carbocycle or heterocycle that is unsubstituted or substituted with one or more R¹⁰.

Embodiment II-226. The compound of any one of embodiments II-218 to II-222, wherein R³ is selected from:

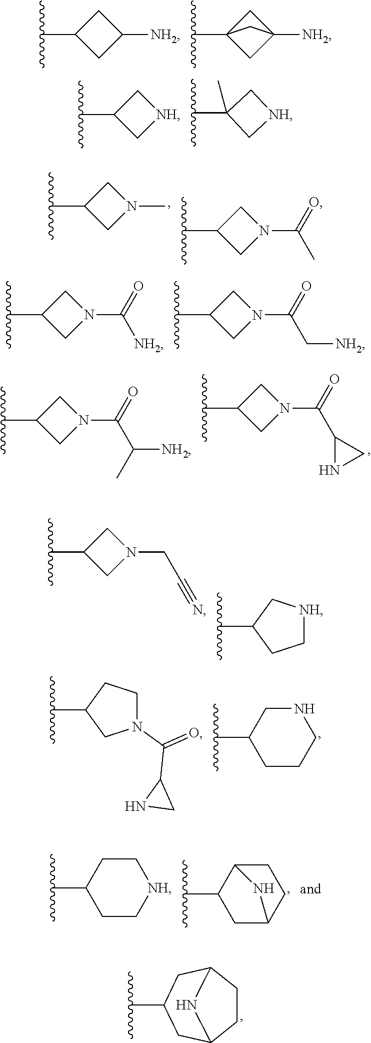

any of which is unsubstituted or substituted with one or more R¹⁰.

Embodiment II-227. The compound of any one of embodiments II-218 to II-221, wherein R² and R³, together with the atom to which they are attached, form a heterocycle that is unsubstituted or substituted with one or more R¹¹.

Embodiment II-228. The compound of any one of embodiments II-218 to II-221, wherein R² and R³, together with the atom to which they are attached, form a heterocycle having the structure:

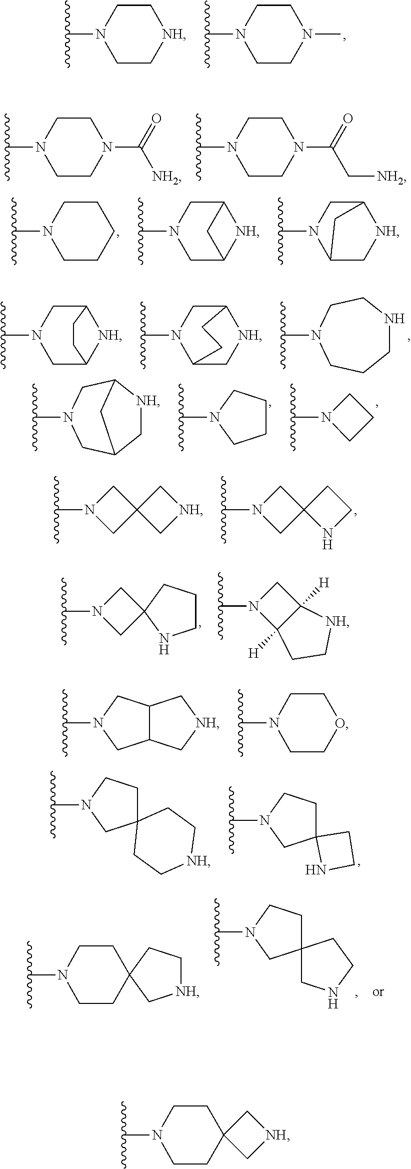

any of which is unsubstituted or substituted with one or more R¹¹.

Embodiment II-229. The compound of any one of embodiments II-218 to II-228, wherein R⁴ is H.

Embodiment II-230. The compound of any one of embodiments II-218 to II-229, wherein R⁵ is a 3-6 membered carbocycle or heterocycle.

Embodiment II-231. The compound of any one of embodiments II-218 to II-229, wherein R⁵ is selected from C₁₋₆alkyl that is unsubstituted or substituted with one or more R¹³.

Embodiment II-232. The compound of any one of embodiments II-218 to II-229, wherein R⁵ is a halogen.

Embodiment II-233. The compound of any one of embodiments II-218 to II-232, wherein R⁷ is a halogen.

Embodiment II-234. The compound of any one of embodiments II-218 to II-233, wherein R⁶ is a bicyclic heteroaryl substituted with one or more R¹⁵.

Embodiment II-235. The compound of embodiment II-234, wherein R⁶ is selected from:

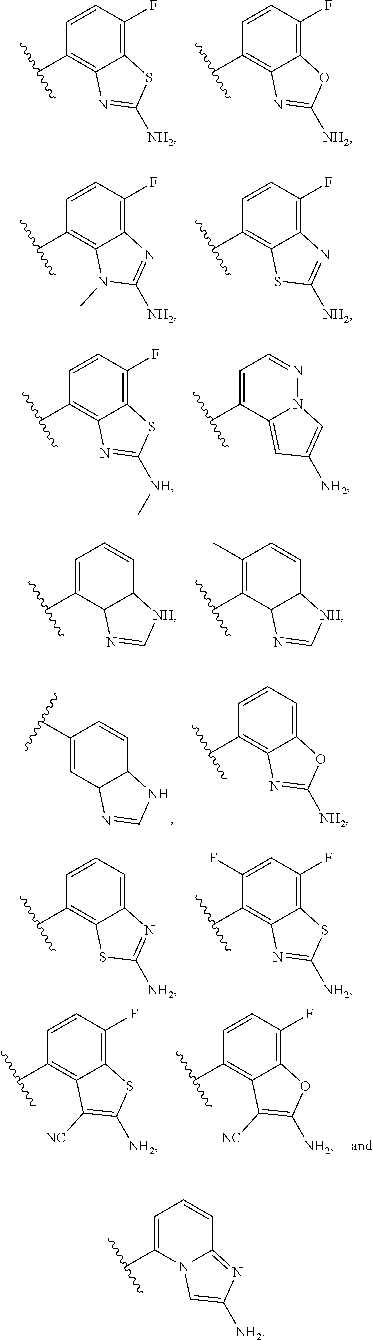

Embodiment II-236. The compound of embodiment II-235, wherein R⁶ is selected from:

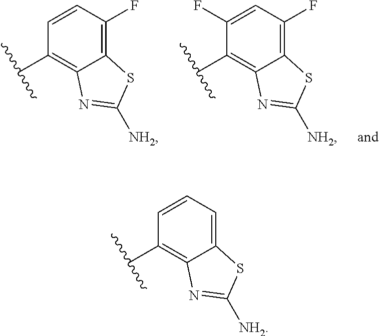 [SMILES: *c1ccc(F)c2sc(N)nc12 ; *c1c(F)cc(F)c2sc(N)nc12 ; *c1cccc2sc(N)nc12]

Embodiment II-237. The compound of embodiment II-218, wherein (i) R⁶ is a bicyclic heteroaryl substituted with one or more R¹⁵ and (ii) R² and R³, together with the atom (e.g., nitrogen atom) to which they are attached, form a heterocycle that is unsubstituted or substituted with one or more R¹¹.

Embodiment II-238. The compound of embodiment II-237, wherein R⁶ is selected from:

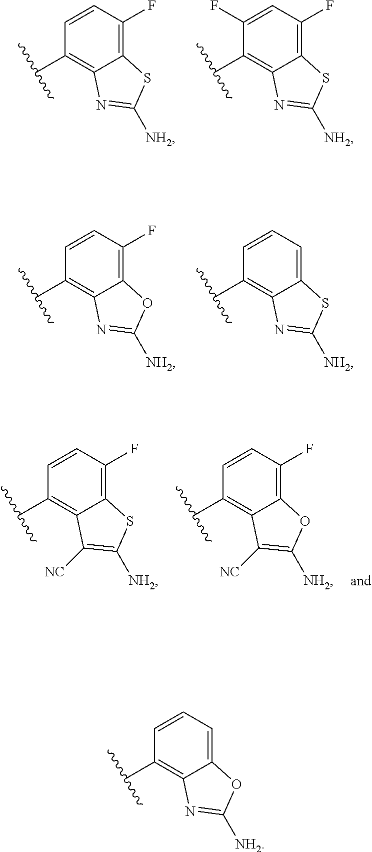

Embodiment II-239. The compound of embodiment II-237 or II-238, wherein R² and R³, together with the atom to which they are attached, form a bridged heterocyclic ring system that is unsubstituted or substituted with one or more R¹¹.

Embodiment II-240. The compound of embodiment II-237 or II-238, wherein R² and R³, together with the atom to which they are attached, form a heterocycle having the structure:

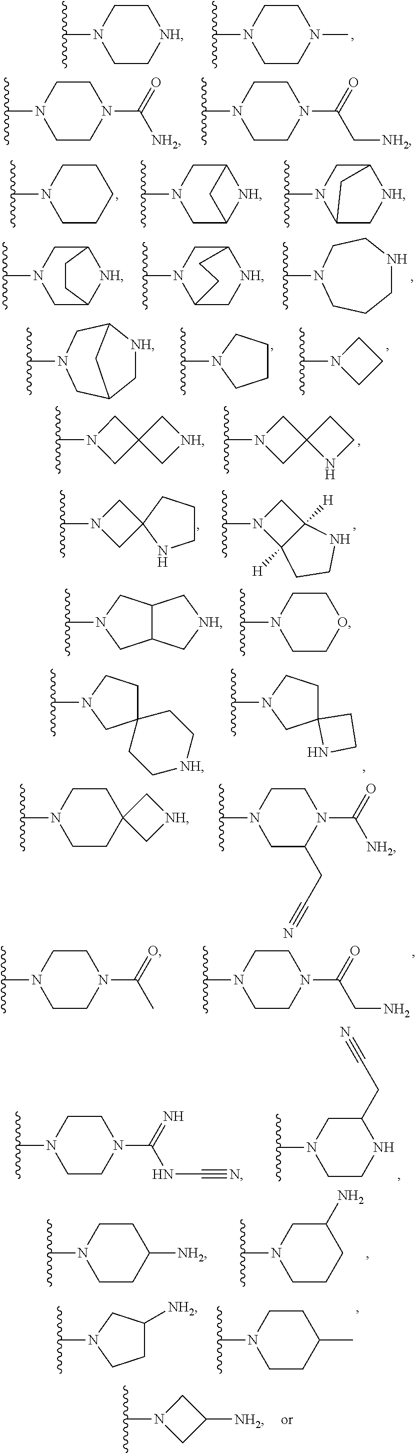

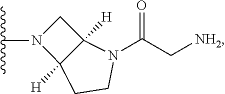 [SMILES: *N1C[C@@H]2[C@H]1CCN2C(=O)CN]

any of which is unsubstituted or substituted with one or more R¹¹.

Embodiment II-241. The compound of embodiment II-237 or II-238, wherein R² and R³, together with the atom to which they are attached, form a heterocycle having the structure:

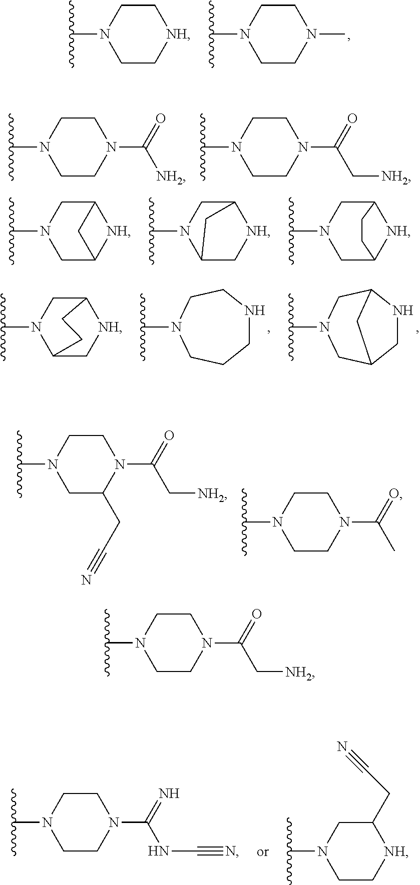

any of which is unsubstituted or substituted with one or more R¹¹.

Embodiment II-242. The compound of embodiment II-237 or II-238, wherein R² and R³, together with the atom to which they are attached, form a heterocycle having the structure:

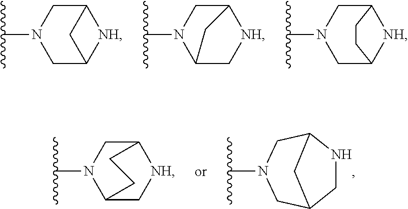

any of which is unsubstituted or substituted with one or more R¹¹.

Embodiment II-243. The compound of any one of embodiments II-237 to II-242, wherein R⁴ is H and/or R⁷ is a halogen.

Embodiment II-244. The compound of embodiment II-218, wherein (i) R⁶ is a bicyclic heteroaryl substituted with one or more R¹⁵; (ii) R² is selected from H, C₁₋₆ alkyl, and a 3-6 membered carbocycle, wherein the C₁₋₆ alkyl or 3-6 membered carbocycle is unsubstituted or is substituted with one or more R¹³; and (iii) R³ is C₁₋₆ alkyl, which C₁₋₆alkyl is substituted with one or more R⁹.

Embodiment II-245. The compound of embodiment II-218, wherein (i) R⁶ is a bicyclic heteroaryl substituted with one or more R¹⁵; (ii) R² is selected from H, C₁₋₆ alkyl, and a 3-6 membered carbocycle, wherein the C₁₋₆ alkyl or 3-6 membered carbocycle is unsubstituted or is substituted with one or more R¹³; and (iii) R³ is a carbocycle, which carbocycle is unsubstituted or substituted with one or more R¹⁰.

Embodiment II-246. The compound of embodiment II-218, wherein (i) R⁶ is a bicyclic heteroaryl substituted with one or more R¹⁵; (ii) R² is selected from H, C₁₋₆ alkyl, and a 3-6 membered carbocycle, wherein the C₁₋₆ alkyl or 3-6 membered carbocycle is unsubstituted or is substituted with one or more R¹³; and (iii) R³ is a heterocycle, which heterocycle is unsubstituted or substituted with one or more R¹⁰.

Embodiment II-247. The compound of embodiment II-246, wherein R³ is selected from:

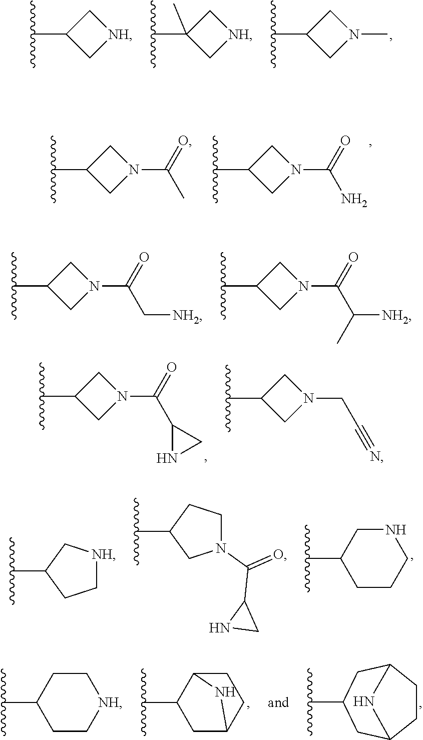

any of which is unsubstituted or substituted with one or more R¹⁰.

Embodiment II-248. A compound according to Formula IF:

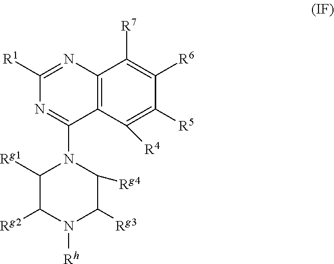

or a salt (e.g., pharmaceutically acceptable salt), ester, tautomer, prodrug, zwitterionic form, or stereoisomer thereof, wherein:
R¹ is selected from —OR⁸, a 4-6 membered heterocycle including a nitrogen atom, and H, wherein a heterocycle is unsubstituted or substituted with one or more R¹⁶; R^{g1}, R^{g2}, R^{g3}, and R^{g4} are each independently selected from H and C₁₋₆alkyl; or (a) R^{g1} and R^{g3} or (b) R^{g2} and R^{g3} join together to form a second ring containing 4-6 members, and any of R^{g1}, R^{g2}, R^{g3}, and R^{g4} that are not part of the second ring are independently selected from H and C₁₋₆alkyl, and wherein any C₁₋₆alkyl is unsubstituted or substituted with one or more R¹³;
R^{h} is selected from H, C₁₋₆alkyl, —C(O)NH₂, and —C(O)C₁₋₆alkylNH₂;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic carbocycle or heterocycle substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-249. The compound of embodiment II-248 wherein at least one of R^{g1}, R^{g2}, R^{g3}, and R^{g4} is selected from C₁₋₆alkyl.

Embodiment II-250. The compound of embodiment II-248, wherein at least two of R^{g1}, R^{g2}, R^{g3}, and R^{g4} are selected from C₁₋₆alkyl.

Embodiment II-251. The compound of embodiment II-248, wherein R^{g1} and R^{g3} join together to form a second ring containing 4-6 members.

Embodiment II-252. The compound of embodiment II-248, wherein R^{g2} and R^{g3} join together to form a second ring containing 4-6 members.

Embodiment II-253. The compound of any one of embodiments II-248 to II-252, wherein R^{h} is H.

Embodiment II-254. The compound of any one of embodiments II-248 to II-253, wherein the compound has the structure:

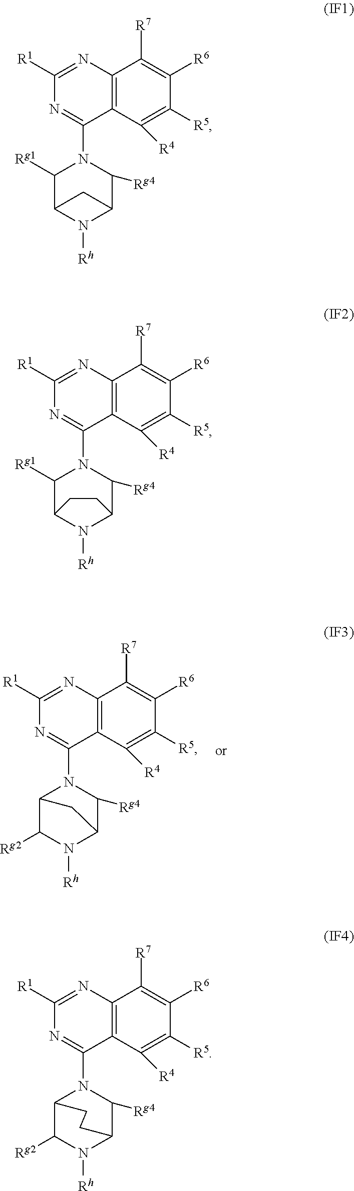

Embodiment II-255. The compound of embodiment II-254, wherein R^{h} is H.

Embodiment II-256. The compound of any one of embodiments II-248 to II-255, wherein R¹ is —OR⁸.

Embodiment II-257. The compound of embodiment II-256, wherein R⁸ is a heterocycle or an alkylheterocycle, wherein the heterocycle of the heterocycle or alkylheterocycle comprises 4-8 members including at least one heteroatom selected from N, O, and S.

Embodiment II-258. The compound of embodiment II-257, wherein R¹ is selected from:

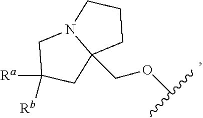

wherein R^{a} and R^{b} are each independently selected from halogen, C₁₋₆alkyl, —OR¹², and H.

Embodiment II-259. The compound of embodiment II-258, wherein R¹ is selected from:

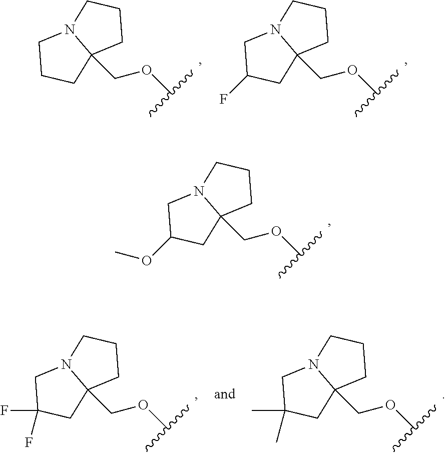 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

Embodiment II-260. The compound of embodiment II-257, wherein R¹ is selected from:

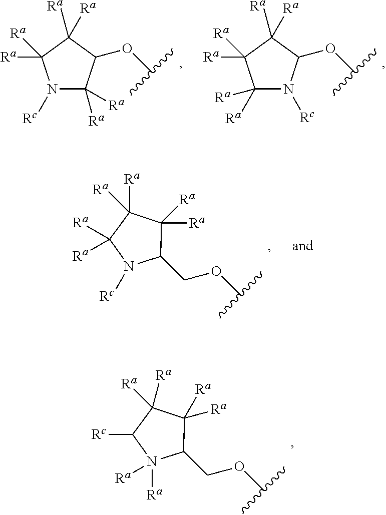

wherein each R^{a} is independently selected from halogen, C₁₋₆ alkyl, —OR¹², and H; and wherein R^{c} is selected from C₁₋₆ alkyl, wherein the C₁₋₆ alkyl is unsubstituted or is substituted with one or more R¹³.

Embodiment II-261. The compound of embodiment II-257, wherein R¹ is selected from:

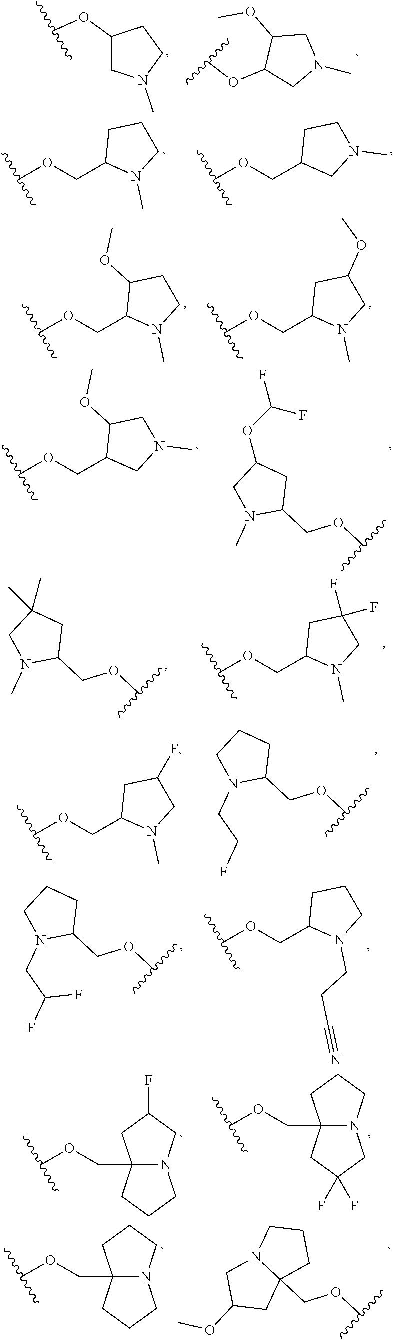

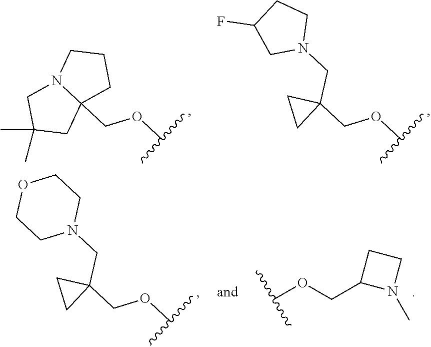

Embodiment II-262. The compound of any one of embodiments II-248 to II-255, wherein R¹ is a 4-6 membered heterocycle including a nitrogen atom, which heterocycle is unsubstituted or substituted with one or more R¹⁶.

Embodiment II-263. The compound of embodiment II-262, wherein R¹ is selected from:

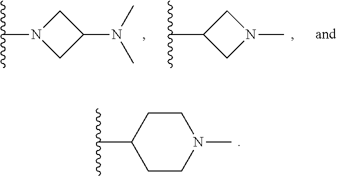

Embodiment II-264. The compound of any one of embodiments II-248 to II-255, wherein R¹ is H.

Embodiment II-265. The compound of any one of embodiments II-248 to II-264, wherein R⁴ is H.

Embodiment II-266. The compound of any one of embodiments II-248 to II-265, wherein R⁵ is a 3-6 membered carbocycle or a 3-6 membered heterocycle.

Embodiment II-267. The compound of any one of embodiments II-248 to II-265, wherein R⁵ is selected from C₁₋₆alkyl that is unsubstituted or substituted with one or more R¹³.

Embodiment II-268. The compound of any one of embodiments II-248 to II-265, wherein R⁵ is selected from —OR¹², where R¹² is selected from C₁₋₆alkyl that is unsubstituted or substituted with one or more R¹³.

Embodiment II-269. The compound of any one of embodiments II-248 to II-265, wherein R⁵ is a halogen.

Embodiment II-270. The compound of any one of embodiments II-248 to II-269, wherein R⁶ is a bicyclic heteroaryl substituted with one or more R¹⁵.

Embodiment II-271. The compound of any one of embodiments II-248 to II-269, wherein R⁶ is selected from:

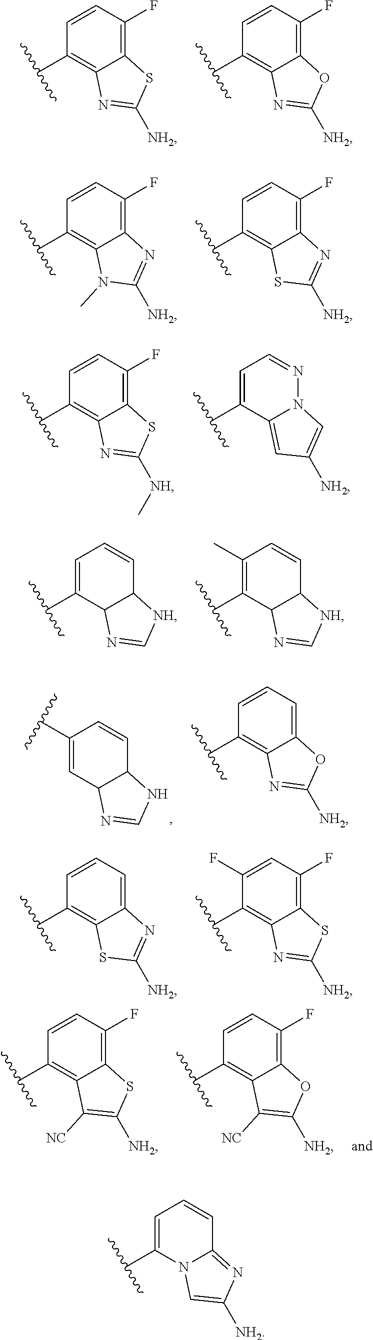

Embodiment II-272. The compound of embodiment II-271, wherein R⁶ is selected from:

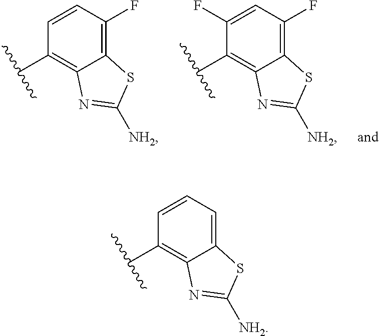 [SMILES: *c1ccc(F)c2sc(N)nc12 ; *c1c(F)cc(F)c2sc(N)nc12 ; *c1cccc2sc(N)nc12]

Embodiment II-273. A compound according to Formula II:

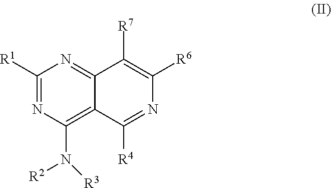

or a salt (e.g., pharmaceutically acceptable salt), ester, tautomer, prodrug, zwitterionic form, or stereoisomer thereof, wherein:
R¹ is selected from —OR⁸, a 4-6 membered heterocycle comprising a nitrogen atom, and H, wherein a heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰; each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment II-274. The compound of embodiment II-273, wherein R³ is selected from a carbocycle, and a heterocycle, wherein the carbocycle or heterocycle is unsubstituted or substituted with one or more R¹⁰.

Embodiment II-275. The compound of embodiment II-274, wherein R³ is selected from:

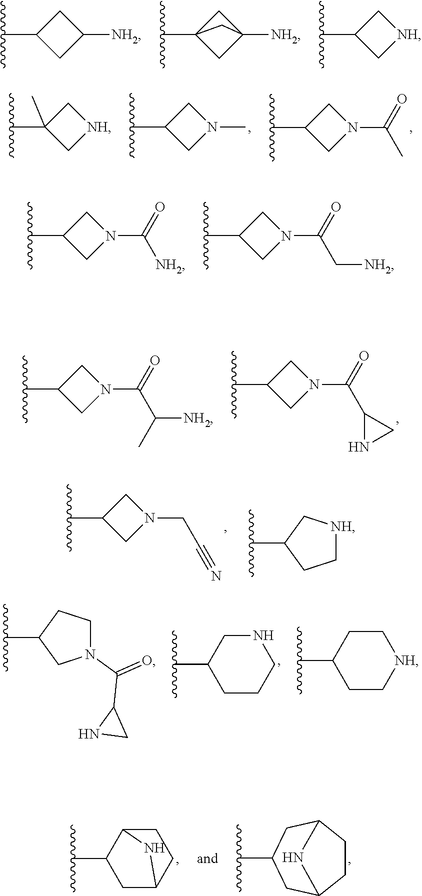

any of which is unsubstituted or substituted with one or more R¹⁰.

Embodiment II-276. The compound of embodiment II-273, wherein R³ is selected from C₁₋₆ alkyl that is substituted with one or more R⁹.

Embodiment II-277. The compound of embodiment II-276, wherein R³ is selected from:

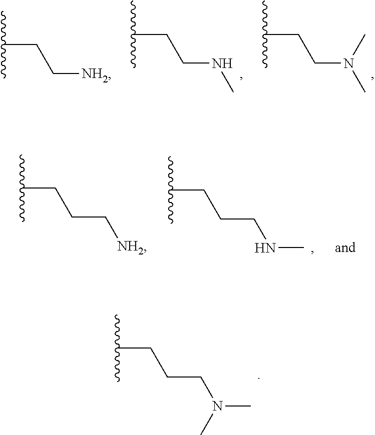

Embodiment II-278. The compound of embodiment II-276, wherein R³ is selected from C₁₋₆alkyl-N(R¹⁷)C(O)C₁₋₆alkylN(R¹⁷)₂.

Embodiment II-279. The compound of any one of embodiments II-273 to II-278, wherein R³ includes an amino moiety.

Embodiment II-280. The compound of embodiment II-273, wherein R² and R³, together with the atom to which they are attached, form a 4-6 membered heterocycle that is unsubstituted or substituted with one or more R¹¹.

Embodiment II-281. The compound of embodiment II-273, wherein R² and R³, together with the atom to which they are attached, form a heterocycle having the structure:

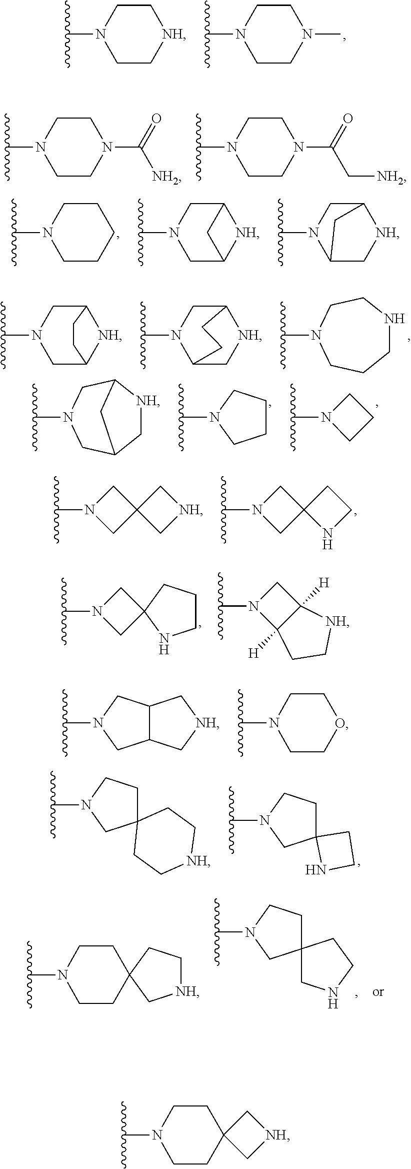

which ring is unsubstituted or substituted with one or more R¹¹.

Embodiment II-282. The compound of embodiment II-273, wherein R² and R³, together with the atom to which they are attached, form a heterocycle having the structure:

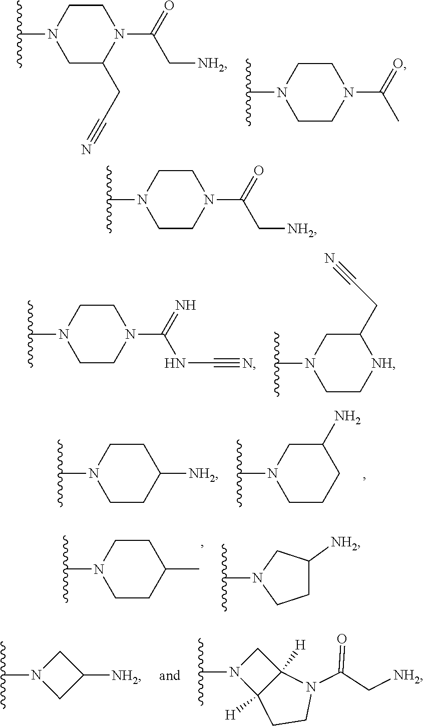

Embodiment II-283. The compound of any one of embodiments II-280 to II-282, wherein R² and R³, together with the atom to which they are attached, form a heterocycle that (i) comprises an additional nitrogen atom and/or (ii) is substituted with a group including an amino moiety.

Embodiment II-284. The compound of any one of embodiments II-273 to II-283, wherein R¹ is H.

Embodiment II-285. The compound of any one of embodiments II-273 to II-283, wherein R¹ is —OR⁸.

Embodiment II-286. The compound of embodiment II-285, wherein R⁸ comprises a 3-8 membered heterocycle that is unsubstituted or substituted with one or more R¹⁴.

Embodiment II-287. The compound of any one of embodiments II-273 to II-283, wherein R¹ is selected from:

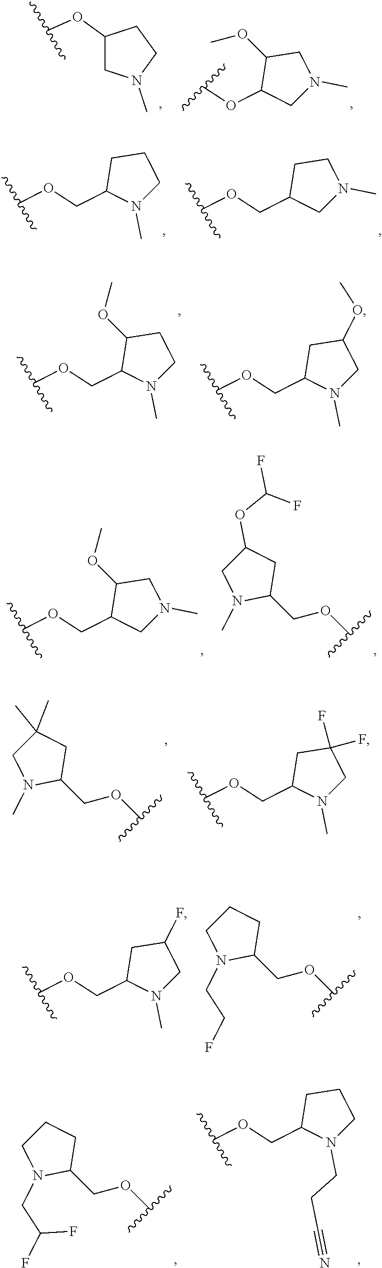

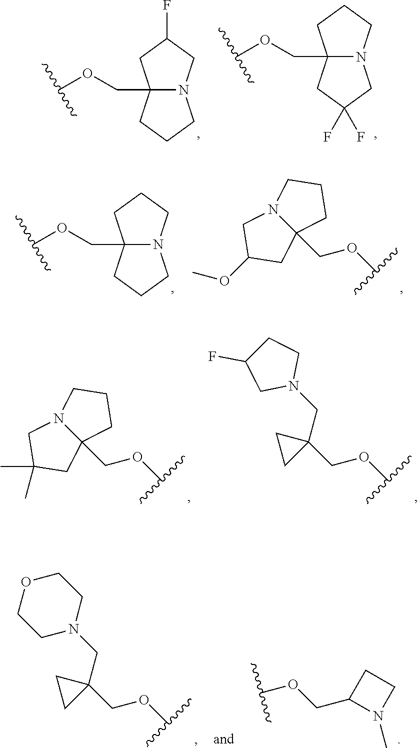

Embodiment II-288. The compound of any one of embodiments II-273 to II-283, wherein R¹ is selected from:

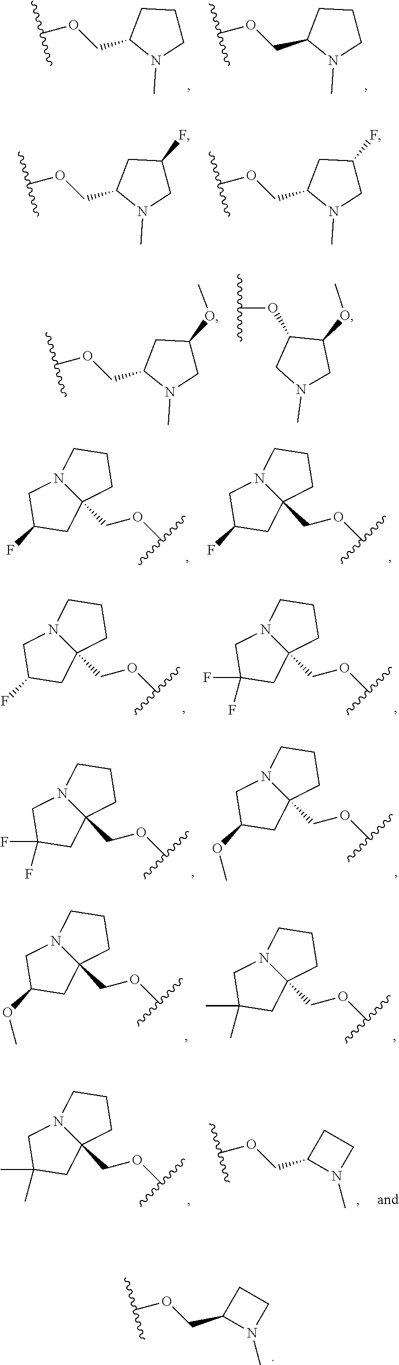

Embodiment II-289. The compound of any one of embodiments II-273 to II-283, wherein R¹ is a 4-6 membered heterocycle comprising a nitrogen atom that is unsubstituted or substituted with one or more R¹⁶.

Embodiment I-290. The compound of embodiment II-289 wherein R¹ is selected from:

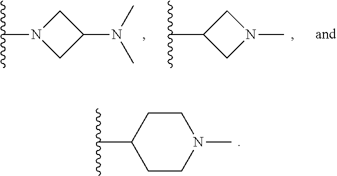

Embodiment II-291. The compound of any one of embodiments II-273 to II-290, wherein R⁶ is selected from:

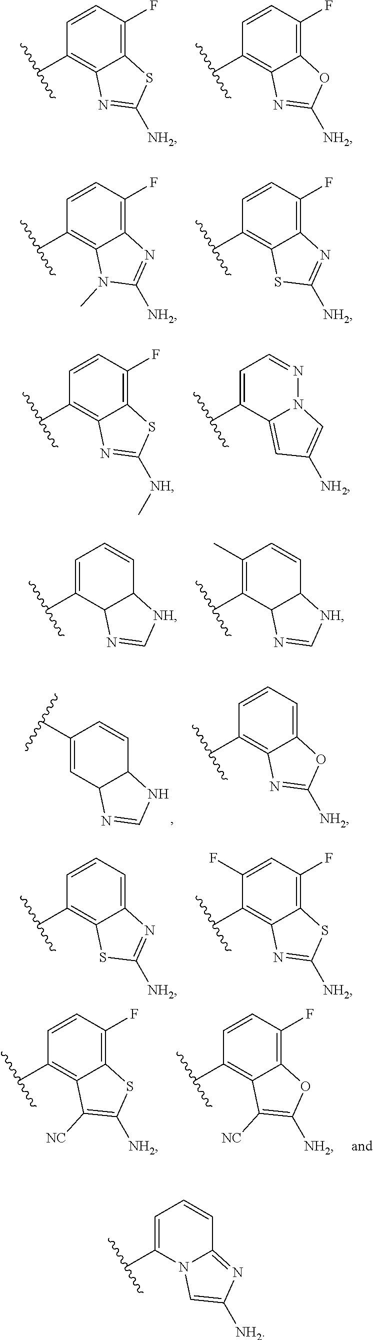

Embodiment II-292. The compound of embodiment II-291, wherein R⁶ is selected from:

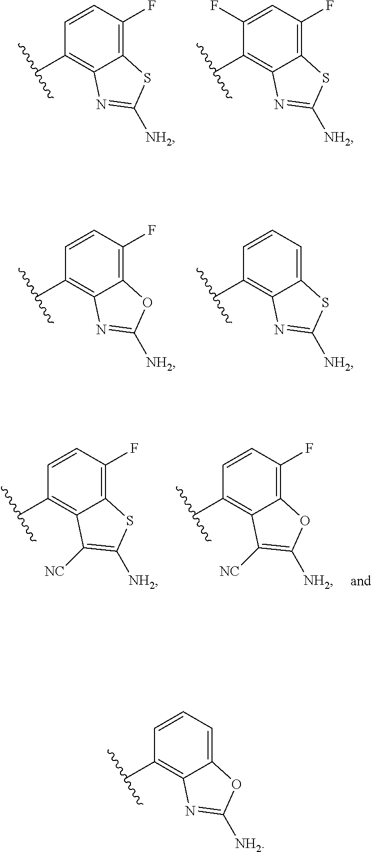

Embodiment II-293. The compound of embodiment II-292, wherein R⁶ is:

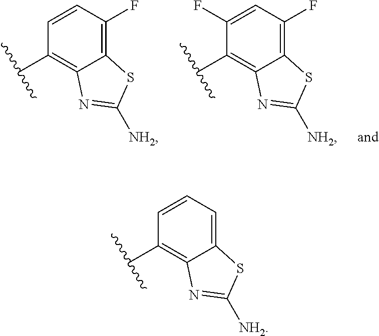 [SMILES: *c1ccc(F)c2sc(N)nc12 ; *c1c(F)cc(F)c2sc(N)nc12 ; *c1cccc2sc(N)nc12]

Embodiment II-294. The compound of any one of embodiments II-273 to II-293, wherein R⁴ is H.

Embodiment II-295. The compound of any one of embodiments II-273 to II-293, wherein R⁴ is a halogen.

Embodiment II-296. The compound of any one of embodiments II-273 to II-295, wherein R⁷ is a halogen.

Embodiment II-297. A compound shown in any one of Tables 2-4, or a salt (e.g., pharmaceutically acceptable salt), ester, tautomer, prodrug, zwitterionic form, or stereoisomer thereof.

Embodiment II-298. The compound of embodiment II-297, wherein the compound is shown in Table 2.

Embodiment II-299. The compound of embodiment II-297, wherein the compound is shown in Table 3.

Embodiment II-300. The compound of embodiment II-297, wherein the compound is shown in Table 4.

Embodiment II-301. A compound shown in Table 7 or 8, or a salt (e.g., pharmaceutically acceptable salt), ester, tautomer, prodrug, zwitterionic form, or stereoisomer thereof.

Embodiment II-302. The compound of embodiment II-301, wherein the compound is shown in Table 7.

Embodiment II-303. The compound of embodiment II-301, wherein the compound is shown in Table 8.

Embodiment II-304. A pharmaceutical composition comprising a compound of any one of embodiments II-1 to II-303, or a salt (e.g., pharmaceutically acceptable salt), ester, tautomer, prodrug, zwitterionic form, or stereoisomer thereof, and a pharmaceutically acceptable excipient.

Embodiment II-305. A compound of any one of embodiments II-1 to II-303, or a salt (e.g., pharmaceutically acceptable salt), ester, tautomer, prodrug, zwitterionic form, or stereoisomer thereof, for use as a medicament.

Embodiment II-306. The compound of embodiment II-305, wherein the medicament is useful in the prevention or treatment of a disease, disorder, or condition ameliorated by the inhibition of KRAS having a G12D mutation.

Embodiment II-307. The compound of embodiment II-305 or II-306, wherein the medicament is useful in the prevention or treatment of a cancer.

Embodiment II-308. The compound of embodiment II-307, wherein the cancer is selected from the group consisting of pancreatic cancer, colorectal cancer, and lung cancer.

Embodiment II-309. A compound of any one of embodiments II-1 to II-303, or a salt (e.g., pharmaceutically acceptable salt), ester, tautomer, prodrug, zwitterionic form, or stereoisomer thereof, for use in the treatment of a disease, disorder, or condition.

Embodiment II-310. The compound of embodiment II-309, wherein the disease, disorder, or condition is a cancer.

Embodiment II-311. The compound of embodiment II-310, wherein the cancer is selected from the group consisting of pancreatic cancer, colorectal cancer, and lung cancer.

Embodiment II-312. The compound of any one of embodiments II-309 to II-311, wherein the compound is used in the treatment of a disease, disorder, or condition in a subject in need thereof.

Embodiment II-313. A compound of any one of embodiments II-1 to II-303, or a salt (e.g., pharmaceutically acceptable salt), ester, tautomer, prodrug, zwitterionic form, or stereoisomer thereof, for use in the manufacture of a medicament.

Embodiment II-314. The compound of embodiment II-313, wherein the medicament is useful in the prevention or treatment of a disease, disorder, or condition ameliorated by the inhibition of KRAS having a G12D mutation.

Embodiment II-315. The compound of embodiment II-313 or II-314, wherein the medicament is useful in the treatment of a cancer.

Embodiment II-316. The compound of embodiment II-315, wherein the cancer is selected from the group consisting of pancreatic cancer, colorectal cancer, and lung cancer.

Embodiment II-317. A method, comprising administering a therapeutically effective amount of a compound of any one of embodiments II-1 to II-303, or a salt (e.g., pharmaceutically acceptable salt), ester, tautomer, prodrug, zwitterionic form, or stereoisomer thereof, to a subject in need thereof.

Embodiment II-318. The method of embodiment II-317, wherein the subject has a disease, disorder, or condition ameliorated by the inhibition of KRAS having a G12D mutation.

Embodiment II-319. The method of embodiment II-317 or II-318, wherein the subject has a cancer.

Embodiment II-320. The method of embodiment II-319, wherein the subject was previously diagnosed with the cancer.

Embodiment II-321. The method of embodiment II-319, wherein the subject has previously undergone a treatment regimen for the cancer.

Embodiment II-322. The method of embodiment II-319, wherein the subject has previously entered remission from the cancer.

Embodiment II-323. The method of any one of embodiments II-319 to II-322, wherein the cancer is selected from the group consisting of pancreatic cancer, colorectal cancer, and lung cancer.

Embodiment II-324. The method of any one of embodiments II-317 to II-323, wherein the compound, or the salt, ester, tautomer, prodrug, zwitterionic form, or stereoisomer thereof, is administered in combination with an additional therapeutic agent.

Embodiment II-325. The use of a compound of any one of embodiments II-1 to II-303, or a salt (e.g., pharmaceutically acceptable salt), ester, tautomer, prodrug, zwitterionic form, or stereoisomer thereof, for the manufacture of a medicament for the treatment of a cancer.

Embodiment II-326. The use of embodiment II-325, wherein the cancer is selected from the group consisting of pancreatic cancer, colorectal cancer, and lung cancer.

Embodiment II-327. A method, comprising contacting a KRAS protein with a compound of any one of embodiments II-1 to II-303, or a salt (e.g., pharmaceutically acceptable salt), ester, tautomer, prodrug, zwitterionic form, or stereoisomer thereof.

Embodiment II-328. The method of embodiment II-327, wherein contacting the KRAS protein with the compound modulates KRAS.

Embodiment II-329. The method of embodiment II-327 or II-328, wherein the KRAS protein has a G12D mutation.

Embodiment II-330. The method of any one of embodiments II-327 to II-329, wherein the KRAS protein is in an active state.

Embodiment II-331. The method of any one of embodiments II-327 to II-329, wherein the KRAS protein is in an inactive state.

Embodiment II-332. The compound of any one of embodiments II-1 to II-303, or a salt (e.g., a pharmaceutically acceptable salt) thereof.

Embodiment III-1. A compound of Formula I:

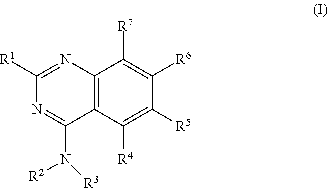

or a salt (e.g., pharmaceutically acceptable salt), ester, tautomer, prodrug, zwitterionic form, or stereoisomer thereof, wherein:
R¹ is selected from —OR⁸, a 4-6 membered heterocycle including a nitrogen atom, and H, wherein a heterocycle is unsubstituted or substituted with one or more R¹⁶;
R² is selected from H and C₁₋₆ alkyl;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic carbocycle or heterocycle substituted with one or more R¹⁵;
R⁷ is selected from halogen, —OR¹², —CN, and H;
R⁸ is selected from H, heterocycle, and alkylheterocycle, wherein the heterocycle or alkylheterocycle is unsubstituted or substituted with one or more R¹⁶;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment III-2. A compound of Formula I.

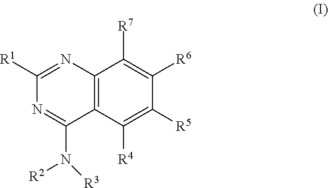

or a salt (e.g., pharmaceutically acceptable salt), ester, tautomer, prodrug, zwitterionic form, or stereoisomer thereof, wherein:
R¹ is selected from —OR⁸, a 4-6 membered heterocycle including a nitrogen atom, and H, wherein a heterocycle is unsubstituted or substituted with one or more R¹⁶;
R² is selected from H, a 3-6 membered carbocycle, and C₁₋₆ alkyl, wherein the 3-6 membered carbocycle or the C₁₋₆ alkyl is unsubstituted or substituted with one or more R¹³;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic carbocycle or heterocycle substituted with one or more R¹⁵;
R⁷ is selected from halogen, —OR¹², —CN, and H;
R⁸ is selected from H, heterocycle, and alkylheterocycle, wherein the heterocycle or alkylheterocycle is unsubstituted or substituted with one or more R¹⁶, and wherein an alkyl moiety of any alkylheterocycle is selected from C₁₋₆ alkyl;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

Embodiment III-3. The compound of embodiment III-1 or III-2, wherein R¹ is —OR⁸.

Embodiment III-4. The compound of embodiment III-3, wherein R⁸ is a heterocycle.

Embodiment III-5. The compound of embodiment III-3, wherein R⁸ is an alkylheterocycle.

Embodiment III-6. The compound of embodiment III-4 or III-5, wherein R⁸ comprises a heterocycle (i) comprising 4-8 members including at least one heteroatom selected from N, O, and S and/or (ii) comprising at least one nitrogen atom.

Embodiment III-7. The compound of any one of embodiments III-4 to III-6, wherein the heterocycle comprises one or more R¹⁶ substituents.

Embodiment III-8. The compound of embodiment III-7, wherein at least one R¹⁶ is —OR¹², wherein R¹² is independently selected from C₁₋₆ alkyl, C₂₋₆ alkenyl, and H.

Embodiment III-9. The compound of embodiment III-8, wherein at least one R¹⁶ is —OCH₃.

Embodiment III-10. The compound of embodiment III-1, wherein R¹ is selected from:

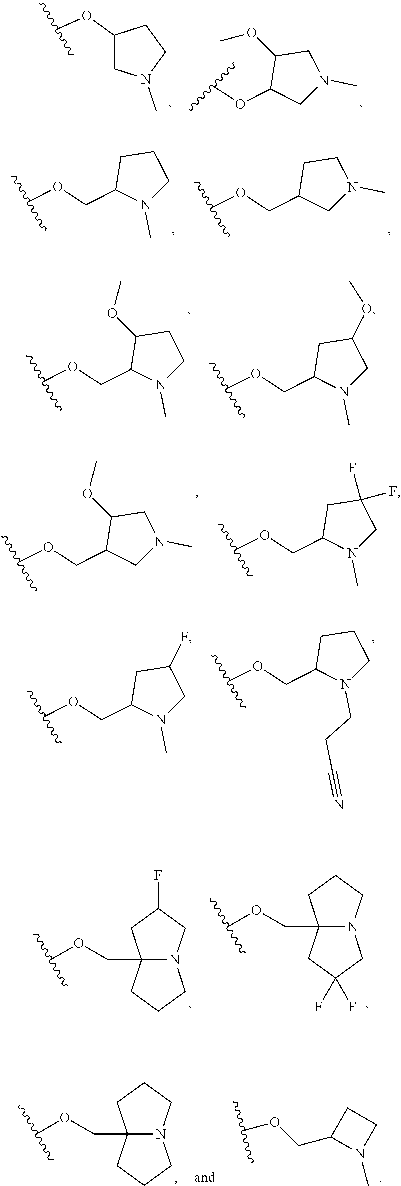

Embodiment III-11. The compound of embodiment III-2, wherein R¹ is selected from:

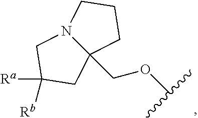

wherein R^{a} and R^{b} are each independently selected from halogen, C₁₋₆alkyl, —OR¹², and H, wherein any C₁₋₆alkyl is unsubstituted or is substituted with one or more R¹³.

Embodiment III-12. The compound of embodiment III-11, wherein R^{a} is a halogen (e.g., F).

Embodiment III-13. The compound of embodiment III-11, wherein R^{a} is C₁₋₆alkyl that is unsubstituted or is substituted with one or more R¹³.

Embodiment III-14. The compound of embodiment III-11, wherein R^{a} is —OC₁₋₆alkyl.

Embodiment III-15. The compound of embodiment III-11, wherein R^{a} is H.

Embodiment III-16. The compound of any one of embodiments III-11 to III-15, wherein R^{b} is a halogen (e.g., F).

Embodiment III-17. The compound of any one of embodiments III-11 to III-15, wherein R^{b} is C₁₋₆alkyl that is unsubstituted or is substituted with one or more R¹³.

Embodiment III-18. The compound of any one of embodiments III-11 to III-15, wherein R^{b} is H.

Embodiment III-19. The compound of embodiment III-2, wherein R¹ is selected from:

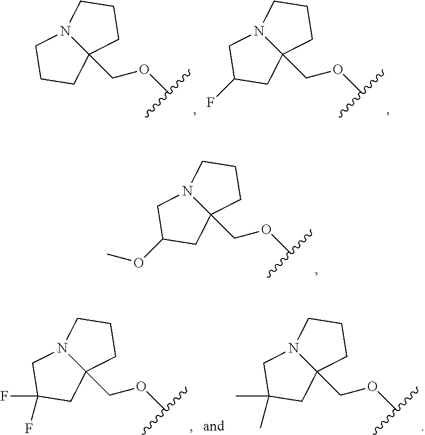

Embodiment III-20. The compound of embodiment III-2, wherein R¹ is selected from:

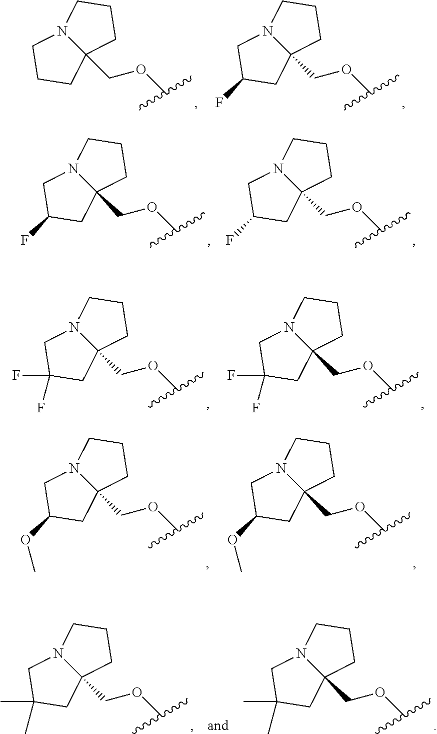

Embodiment III-21. The compound of embodiment III-2, wherein R¹ is selected from:

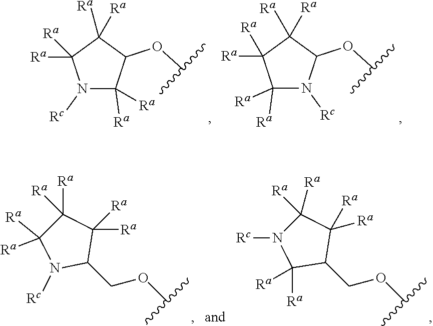

wherein each R^{a} is independently selected from halogen, C₁₋₆ alkyl, —OR¹², and H; and wherein R^{c} is selected from C₁₋₆ alkyl, wherein a C₁₋₆ alkyl of a R^{a} or R^{c} is unsubstituted or is substituted with one or more R¹³.

Embodiment III-22. The compound of embodiment III-2, wherein R¹ is selected from:

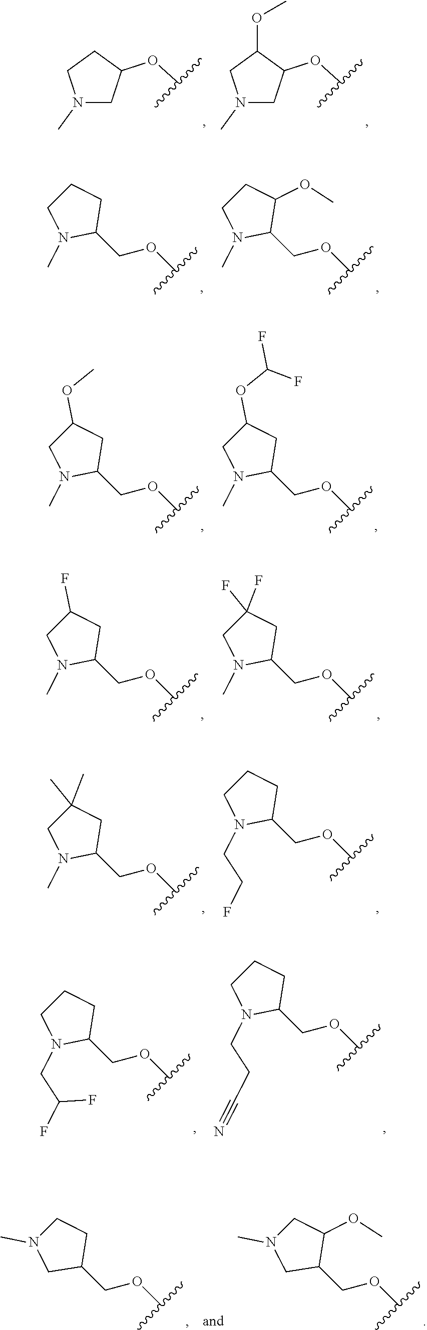

Embodiment III-23. The compound of embodiment III-2, wherein R¹ is selected from:

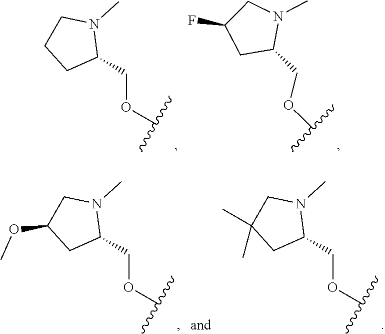

Embodiment III-24. The compound of embodiment III-2, wherein R¹ is selected from:

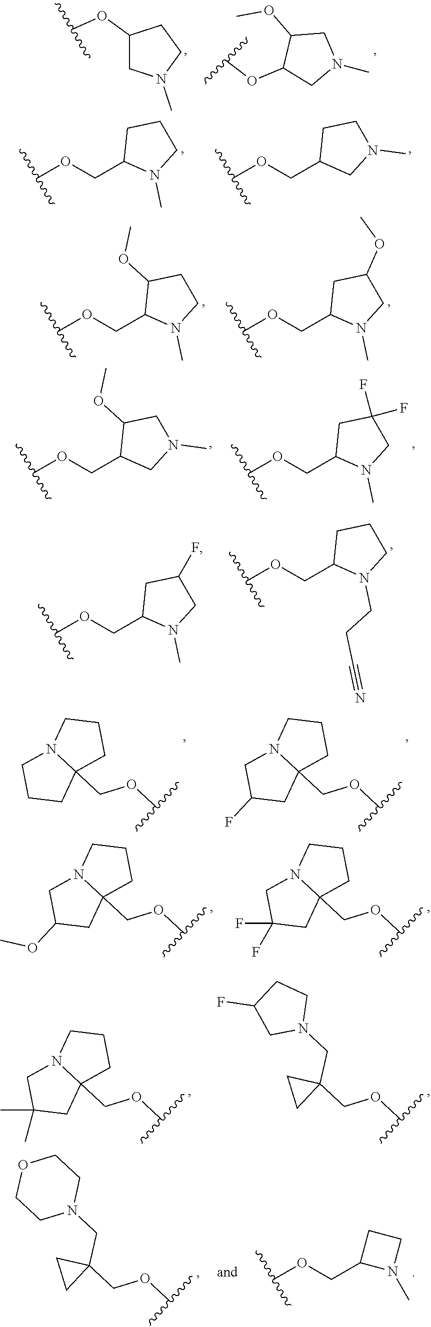

Embodiment III-25. The compound of embodiment III-1 or III-2, wherein R¹ is selected from:

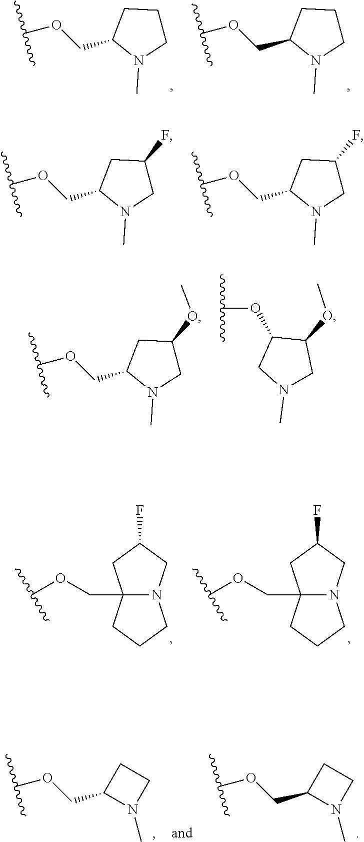

Embodiment III-26. The compound of embodiment III-1 or III-2, wherein R¹ is a 4-6 membered heterocycle comprising a nitrogen atom, which heterocycle is unsubstituted or substituted with one or more R¹⁶.

Embodiment III-27. The compound of embodiment III-26, wherein R¹ is selected from:

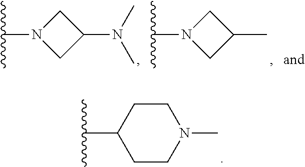

Embodiment III-28. The compound of embodiment III-1 or III-2, wherein R¹ is H.

Embodiment III-29. The compound of any one of embodiments III-1 to III-28, wherein R² is H.

Embodiment III-30. The compound of any one of embodiments III-1 to III-28, wherein R² is C₁₋₆ alkyl.

Embodiment III-31. The compound of any one of embodiments III-2 to III-28, wherein R² is a 3-6 membered carbocycle (e.g., cyclopropyl).

Embodiment III-32. The compound of any one of embodiments III-1 to III-31, wherein R³ is C₁₋₆ alkyl, which C₁₋₆alkyl is substituted with one or more R⁹.

Embodiment III-33. The compound of embodiment III-32, wherein R³ is C₁₋₆ alkyl substituted with —N(R¹⁷)₂, wherein each R¹⁷ is independently selected from C₁₋₆ alkyl and H.

Embodiment III-34. The compound of embodiment III-32, wherein R³ is C₁₋₆ alkyl substituted with —NH₂.

Embodiment III-35. The compound of any one of embodiments III-1 to III-31, wherein R³ is selected from:

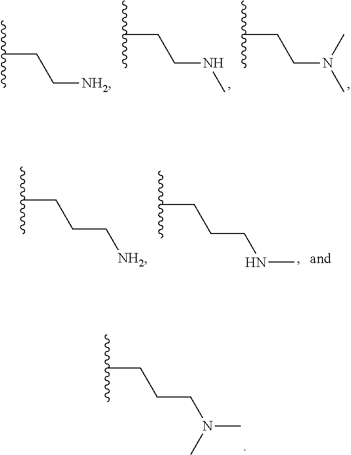

Embodiment III-36. The compound of any one of embodiments III-1 to III-31, wherein R³ is a carbocycle (e.g., a multicyclic or monocyclic carbocycle), which carbocycle is unsubstituted or substituted with one or more R¹⁰.

Embodiment III-37. The compound of embodiment III-36, wherein R³ is a carbocycle that is substituted with —N(R¹⁹)₂, wherein each R¹⁹ is independently selected from unsubstituted or substituted C₁₋₆ alkyl and H.

Embodiment III-38. The compound of embodiment III-37, wherein R³ is a carbocycle that is substituted with —NH₂.

Embodiment III-39. The compound of any one of embodiments III-1 to III-31, wherein R³ is selected from C₁₋₆alkyl-N(R¹⁷)C(O)C₁₋₆alkylN(R¹⁷)₂.

Embodiment III-40. The compound of any one of embodiments III-1 to III-31, wherein R³ is a heterocycle (e.g., a multicyclic or monocyclic heterocycle), which heterocycle is unsubstituted or substituted with one or more R¹⁰.

Embodiment III-41. The compound of embodiment III-40, wherein the heterocycle comprises one or more nitrogen atoms.

Embodiment III-42. The compound of embodiment III-40 or III-41, wherein R³ is a heterocycle that is substituted with —N(R¹⁹)₂, wherein each R¹⁹ is independently selected from unsubstituted or substituted C₁₋₆ alkyl and H.

Embodiment III-43. The compound of embodiment III-42, wherein R³ is a heterocycle that is substituted with —NH₂.

Embodiment III-44. The compound of any one of embodiments III-1 to III-31, wherein R³ is selected from:

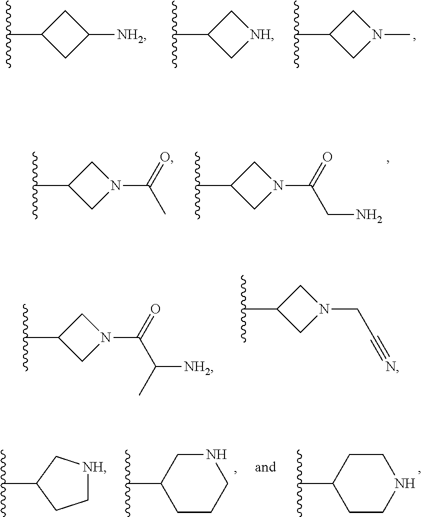

any of which is unsubstituted or substituted with one or more R¹⁰.

Embodiment III-45. The compound of any one of embodiments III-1 to III-31, wherein R³ is selected from:

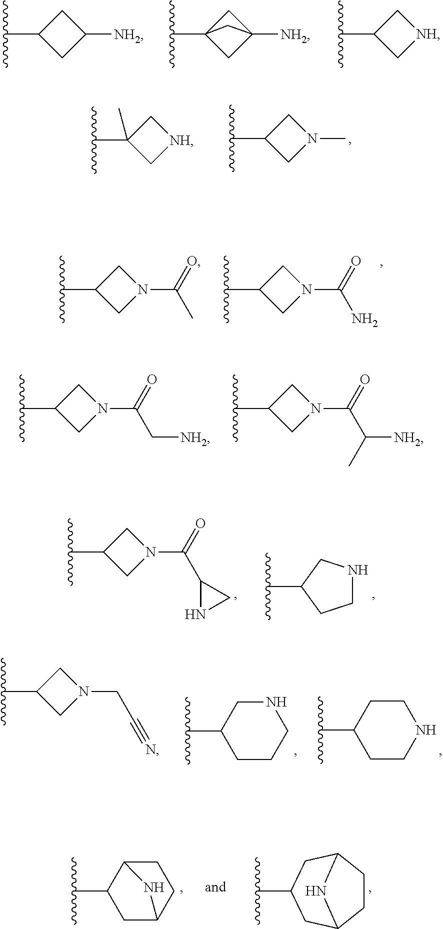

any of which is unsubstituted or substituted with one or more R¹⁰.

Embodiment III-46. The compound of any one of embodiments III-30 to III-45, wherein R² or R³ comprises an amino moiety.

Embodiment III-47. The compound of any one of embodiments III-1 to III-28, wherein R² and R³, together with the atom (e.g., nitrogen atom) to which they are attached, form a heterocycle that is unsubstituted or substituted with one or more R¹¹.

Embodiment III-48. The compound of embodiment III-47, wherein R² and R³, together with the atom to which they are attached, form a heterocycle having the structure:

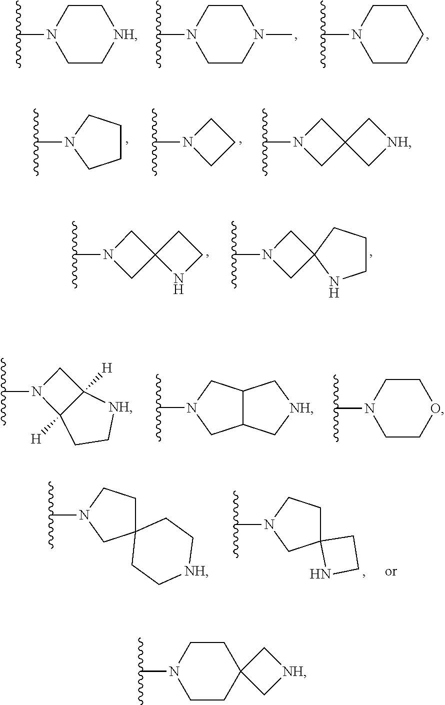

any of which is unsubstituted or substituted with one or more R¹¹.

Embodiment III-49. The compound of embodiment III-47, wherein R² and R³, together with the atom to which they are attached, form a heterocycle having the structure:

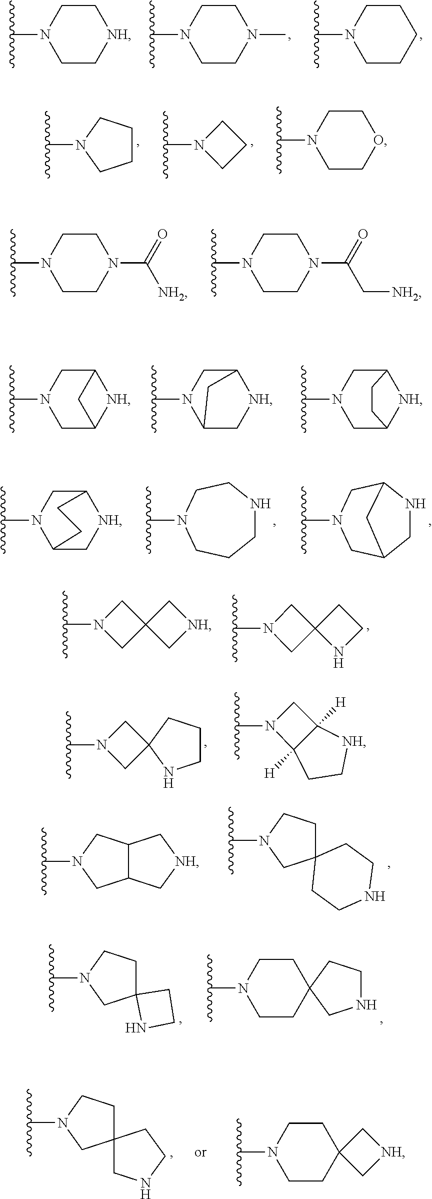

any of which is unsubstituted or substituted with one or more R¹¹.

Embodiment III-50. The compound of embodiment III-47, wherein R² and R³, together with the atom to which they are attached, form a heterocycle having the structure:

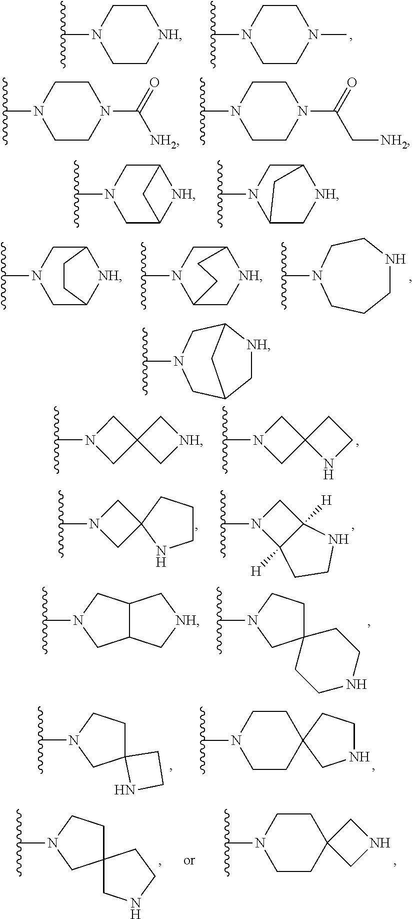

any of which is unsubstituted or substituted with one or more R¹¹.

Embodiment III-51. The compound of embodiment III-47, wherein R² and R³, together with the atom to which they are attached, form a heterocycle having the structure:

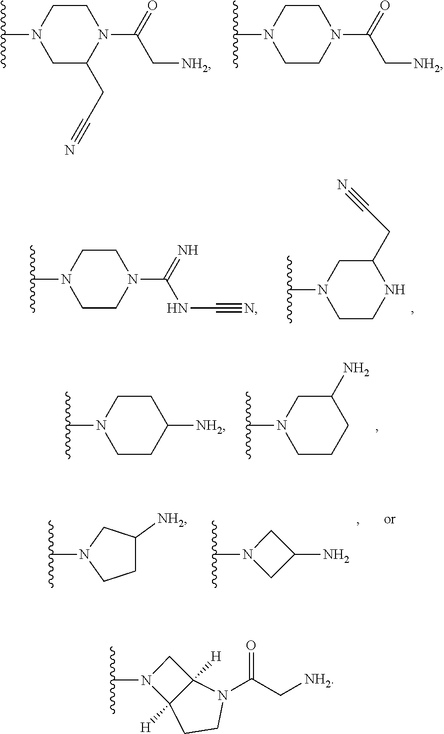

Embodiment III-52. The compound of embodiment III-47, wherein R² and R³, together with the atom to which they are attached, form a heterocycle having the structure:

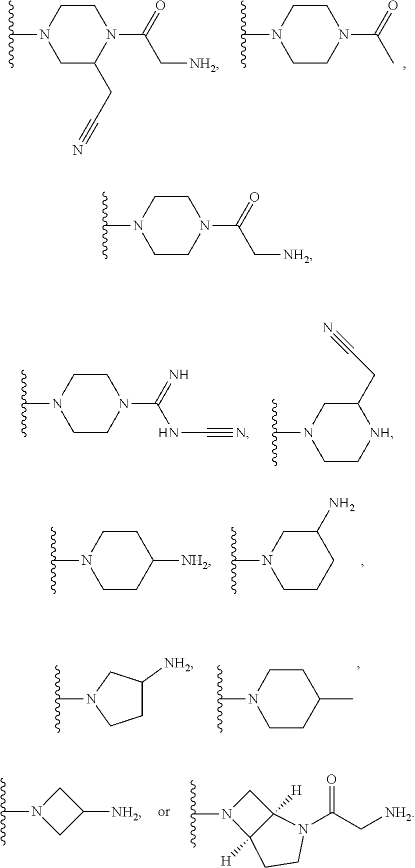

Embodiment III-53. The compound of embodiment III-47, wherein R² and R³, together with the atom to which they are attached, form a heterocycle having the structure:

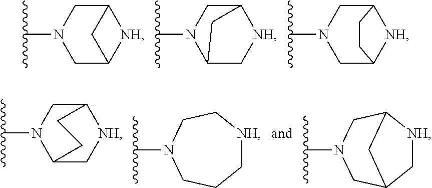

any of which is unsubstituted or substituted with one or more R¹¹.

Embodiment III-54. The compound of embodiment III-47, wherein R² and R³, together with the atom to which they are attached, form a heterocycle having the structure:

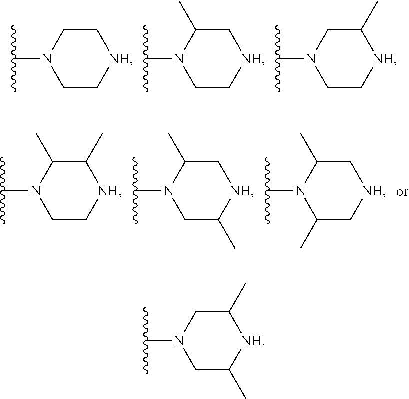

Embodiment III-55. The compound of embodiment III-47, wherein R² and R³, together with the nitrogen atom to which they are attached, form a heterocycle that (i) comprises an additional nitrogen atom or (ii) is substituted with a group comprising an amino moiety.

Embodiment III-56. The compound of any one of embodiments III-1 to III-55, wherein R⁴ is hydrogen.

Embodiment III-57. The compound of any one of embodiments III-1 to III-55, wherein R⁴ is a halogen.

Embodiment III-58. The compound of any one of embodiments III-1 to III-55, wherein R⁴ is —OR¹² (e.g., —OCH₃).

Embodiment III-59. The compound of any one of embodiments III-1 to III-58, wherein R⁵ is a halogen (e.g., Cl or F).

Embodiment III-60. The compound of any one of embodiments III-1 to III-58, wherein R⁵ is a 3-6 membered carbocycle.

Embodiment III-61. The compound of any one of embodiments III-1 to III-58, wherein R⁵ is a 3-6 membered heterocycle.

Embodiment III-62. The compound of any one of embodiments III-1 to III-58, wherein R⁵ is a 5 or 6 membered aryl or heteroaryl moiety (e.g., a furan).

Embodiment III-63. The compound of any one of embodiments III-1 to III-58, wherein R⁵ is selected from C₁₋₆alkyl that is unsubstituted or substituted with one or more R¹³.

Embodiment III-64. The compound of embodiment III-63, wherein R⁵ is selected from C₁₋₆alkyl that is unsubstituted, such as methyl or ethyl.

Embodiment III-65. The compound of embodiment III-63, wherein R⁵ is selected from C₁₋₆alkyl that is substituted with one or more halogens or —CN (e.g., —CF₃, —CF₂H, or —CH₂CN).

Embodiment III-66. The compound of any one of embodiments III-1 to III-8, wherein R⁵ is selected from —OR¹², wherein R¹² is selected from C₁₋₆alkyl that is unsubstituted or substituted with one or more R¹³ (e.g., —OCH₃, —OCF₃, or —OCF₂H).

Embodiment III-67. The compound of any one of embodiments III-1 to III-66, wherein R⁷ is a halogen (e.g., Cl or F).

Embodiment III-68. The compound of any one of embodiments III-1 to III-67, wherein R⁶ is a bicyclic aryl substituted with one or more R¹⁵.

Embodiment III-69. The compound of any one of embodiments III-1 to III-68, wherein R⁶ is selected from:

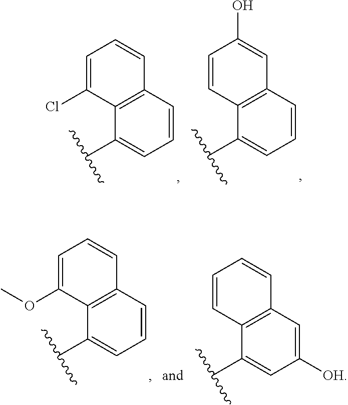

Embodiment III-70. The compound of any one of embodiments III-1 to III-67, wherein R⁶ is a bicyclic heteroaryl substituted with one or more R¹⁵.

Embodiment III-71. The compound of embodiment III-70, wherein R⁶ is selected from:

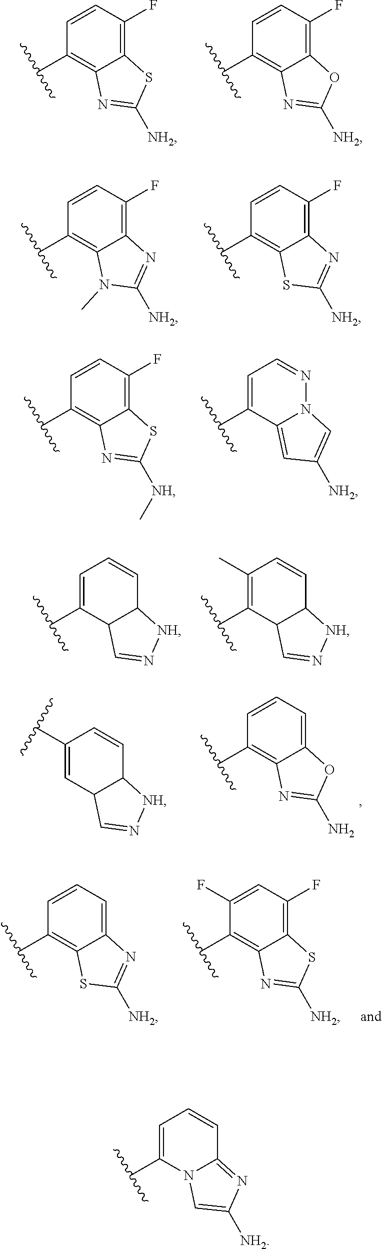

Embodiment III-72. The compound of embodiment III-70, wherein R⁶ is selected from:

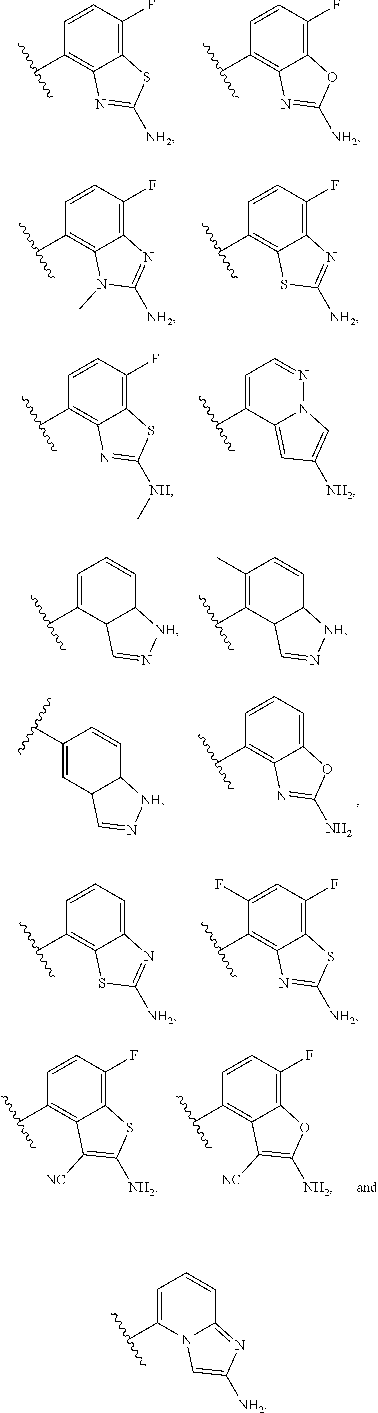

Embodiment III-73. The compound of embodiment III-70, wherein R⁶ is selected from:

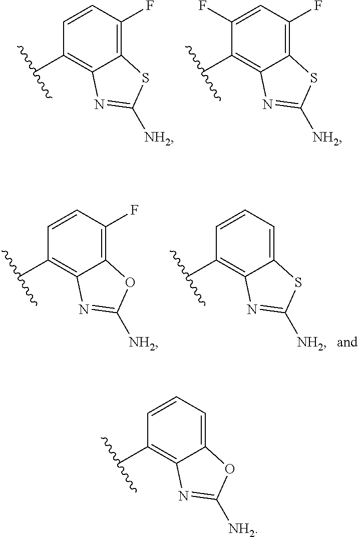 [SMILES: *c1ccc(F)c2sc(N)nc12 ; *c1c(F)cc(F)c2sc(N)nc12 ; *c1ccc(F)c2oc(N)nc12 ; *c1cccc2sc(N)nc12 ; *c1cccc2oc(N)nc12]

Embodiment III-74. The compound of embodiment III-70, wherein R⁶ is selected from:

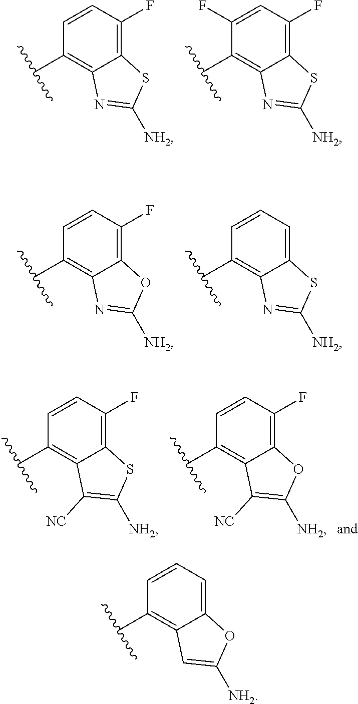

Embodiment III-75. The compound of embodiment III-70, wherein R⁶ is:

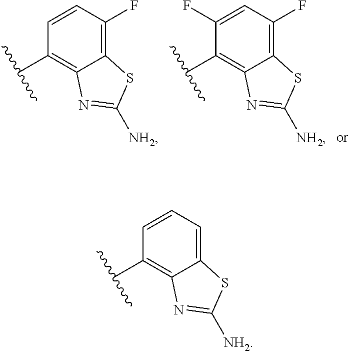 [SMILES: *c1ccc(F)c2sc(N)nc12 ; *c1c(F)cc(F)c2sc(N)nc12 ; *c1cccc2sc(N)nc12]

Embodiment III-76. The compound of embodiment III-70, wherein R⁶ is:

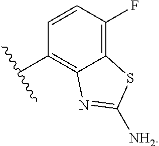 [SMILES: *c1ccc(F)c2sc(N)nc12]

Embodiment III-77. The compound of any one of embodiments III-1 to III-76, wherein (i) R² is selected from C₁₋₆alkyl and a 3-6 membered carbocycle, wherein the C₁₋₆alkyl and a 3-6 membered carbocycle is unsubstituted or is substituted with one or more R¹³; and (ii) R³ is selected from a carbocycle and a heterocycle, wherein the carbocycle or heterocycle is unsubstituted or substituted with one or more R¹⁰.

Embodiment III-78. The compound of any one of embodiments III-1 to III-77, wherein R⁵ is selected from C₁₋₆alkyl, a halogen, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴.

Embodiment III-79. The compound of any one of embodiments III-2 to III-78, wherein R² is selected from C₁₋₆alkyl (e.g., methyl or ethyl) that is unsubstituted or is substituted with one or more R¹³.

Embodiment III-80. The compound of any one of embodiments III-1 to III-79, or a salt (e.g., a pharmaceutically acceptable salt) thereof.

Embodiment III-81. A pharmaceutical composition comprising a compound of any one of embodiments III-1 to III-80, or a salt (e.g., pharmaceutically acceptable salt), ester, tautomer, prodrug, zwitterionic form, or stereoisomer thereof, and a pharmaceutically acceptable excipient.

Embodiment III-81. A compound of any one of embodiments III-1 to III-80, or a salt (e.g., pharmaceutically acceptable salt), ester, tautomer, prodrug, zwitterionic form, or stereoisomer thereof, for use as a medicament.

Embodiment III-82. The compound of embodiment III-81, wherein the medicament is useful in the prevention or treatment of a disease, disorder, or condition ameliorated by the inhibition of KRAS having a G12D mutation.

Embodiment III-83. The compound of embodiment III-81 or III-82, wherein the medicament is useful in the prevention or treatment of a cancer.

Embodiment III-84. The compound of embodiment III-83, wherein the cancer is selected from the group consisting of pancreatic cancer, colorectal cancer, and lung cancer.

Embodiment III-85. A compound of any one of embodiments III-1 to III-80, or a salt (e.g., pharmaceutically acceptable salt), ester, tautomer, prodrug, zwitterionic form, or stereoisomer thereof, for use in the treatment of a disease, disorder, or condition.

Embodiment III-86. The compound of embodiment III-85, wherein the disease, disorder, or condition is a cancer.

Embodiment III-87. The compound of embodiment III-86, wherein the cancer is selected from the group consisting of pancreatic cancer, colorectal cancer, and lung cancer.

Embodiment III-88. The compound of any one of embodiments III-85 to III-87, wherein the compound is used in the treatment of a disease, disorder, or condition in a subject in need thereof.

Embodiment III-89. A compound of any one of embodiments III-1 to III-80, or a salt (e.g., pharmaceutically acceptable salt), ester, tautomer, prodrug, zwitterionic form, or stereoisomer thereof, for use in the manufacture of a medicament.

Embodiment III-90. The compound of embodiment III-89, wherein the medicament is useful in the prevention or treatment of a disease, disorder, or condition ameliorated by the inhibition of KRAS having a G12D mutation.

Embodiment III-91. The compound of embodiment III-89 or III-90, wherein the medicament is useful in the treatment of a cancer.

Embodiment III-92. The compound of embodiment III-91, wherein the cancer is selected from the group consisting of pancreatic cancer, colorectal cancer, and lung cancer.

Embodiment III-93. A method, comprising administering a therapeutically effective amount of a compound of any one of embodiments III-1 to III-80, or a salt (e.g., pharmaceutically acceptable salt), ester, tautomer, prodrug, zwitterionic form, or stereoisomer thereof, to a subject in need thereof.

Embodiment III-94. The method of embodiment III-93, wherein the subject has a disease, disorder, or condition ameliorated by the inhibition of KRAS having a G12D mutation.

Embodiment III-95. The method of embodiment III-93 or III-94, wherein the subject has a cancer.

Embodiment III-96. The method of embodiment III-95, wherein the subject was previously diagnosed with the cancer.

Embodiment III-97. The method of embodiment III-96, wherein the subject has previously undergone a treatment regimen for the cancer.

Embodiment III-98. The method of embodiment III-96, wherein the subject has previously entered remission from the cancer.

Embodiment III-99. The method of any one of embodiments III-95 to III-98, wherein the cancer is selected from the group consisting of pancreatic cancer, colorectal cancer, and lung cancer.

Embodiment III-100. The method of any one of embodiments III-93 to III-99, wherein the compound, or the salt, ester, tautomer, prodrug, zwitterionic form, or stereoisomer thereof, is administered in combination with an additional therapeutic agent.

Embodiment III-101. The use of a compound of any one of embodiments III-1 to III-80, or a salt (e.g., pharmaceutically acceptable salt), ester, tautomer, prodrug, zwitterionic form, or stereoisomer thereof, for the manufacture of a medicament for the treatment of a cancer.

Embodiment III-102. The use of embodiment III-101, wherein the cancer is selected from the group consisting of pancreatic cancer, colorectal cancer, and lung cancer.

Embodiment III-103. A method, comprising contacting a KRAS protein with a compound of any one of embodiments III-1 to III-80, or a salt (e.g., pharmaceutically acceptable salt), ester, tautomer, prodrug, zwitterionic form, or stereoisomer thereof.

Embodiment III-104. The method of embodiment III-103, wherein contacting the KRAS protein with the compound modulates KRAS.

Embodiment III-105. The method of embodiment III-103 or III-104, wherein the KRAS protein has a G12D mutation.

Embodiment III-106. The method of any one of embodiments III-103 to III-105, wherein the KRAS protein is in an active state.

Embodiment III-107. The method of any one of embodiments III-103 to III-105, wherein the KRAS protein is in an inactive state.

### EXAMPLES

Selected abbreviations used in the preceding sections and the Examples are summarized in Table 1.

TABLE 1
Abbreviations.

| Abbreviation | Term |
|---|---|
| MeCN | acetonitrile |
| cm | centimeter |
| ° C. | degrees Celsius |
| ° K | degrees Kelvin |
| DCM | dichloromethane |
| DMF | dimethylformamide |
| DMSO | dimethyl sulfoxide |
| ESI | electrospray ion-mass spectrometry |
| EtOH | ethanol |
| EtOAc, EA | ethyl acetate |
| g | gram |
| Hz | hertz |
| HPLC | high performance liquid chromatography |
| HTRF | homogenous time-resolved fluorescence |
| h | hour |
| LC | liquid chromatography |
| LCMS | liquid chromatography-mass spectrometry |
| L | liter |
| MS | mass spectra |
| MHz | megahertz |
| MeOH | methanol |
| MTBE | methyl tert-butyl ether |
| μg | microgram |
| μL | microliter |
| μM | micromolar |
| μm | micron |
| μs | microsecond |
| mg | milligram |
| mL | milliliter |
| mm | millimeter |
| mM | millimolar |
| mmol | millimole |
| min | minute |
| M | molar |
| nL | nanoliter |
| nm | nanometer |
| NCS | N-chlorosuccinimide |
| NMP | N-methyl-2-pyrrolidone |
| DIPEA | N-N-diisopropylethylamine |
| ppm | parts per million |
| PTLC | preparative thin layer chromatography |
| ¹HNMR | proton nuclear magnetic resonance |
| RBD | receptor binding domain |
| RP | reverse phase |
| rpm | revolutions per minute |
| SPR | surface plasmon resonance |
| THF | tetrahydrofuran |
| TMS | tetramethylsilane |
| TLC | thin layer chromatography |
| SOCl₂ | thionyl chloride |
| TEA | triethylamine |
| TFA | trifluoroacetic acid |
| TCEP | tris(2-carboxyethyl)phosphine |
| UV | ultraviolet |
| UV/Vis | ultraviolet/visible |

### Materials and Methods

Preparative thin layer chromatography (PTLC) separations described herein were typically performed on 20×20 cm plates (500-μm thick silica gel).

Chromatographic purifications were typically performed using Biotage Isolera. One automated system running Biotage Isolera One 2.0.6 software (Biotage LLC, Charlotte, NC). Flow rates were the default values specified for the column in use. Reverse phase chromatography was performed using elution gradients of water and acetonitrile on KP-C18-HS Flash+ columns (Biotage LLC) of various sizes. Typical loading was between 1:50 and 1:1000 crude sample: RP SiO₂ by weight. Normal phase chromatography was performed using elution gradients of various solvents (e.g. hexane, ethyl acetate, methylene chloride, methanol, acetone, chloroform, MTBE, etc.). The columns were SNAP Cartridges containing KP-SIL or SNAP Ultra (25 pm spherical particles) of various sizes (Biotage LLC). Typical loading was between 1:10 to 1:150 crude sample: SiO₂ by weight. Alternatively, silica gel chromatography was performed on a Biotage Horizon flash chromatography system.

¹HNMR analyses of intermediates and exemplified compounds were typically performed on a Bruker Ascend™ 400 spectrometer (operating at 400 MHz), Bruker Ascend 700 MHz Advance Neo Spectrometer (Bruker-Biospin) or Bruker Advance ultrashield 300/54 (operating at 300 MHz) at 298° K following standard operating procedure suggested by manufacturer. Reference frequency was set using TMS as an internal standard. Chemical shift values (δ) are reported in parts per million (ppm) with splitting patterns abbreviated to: s (singlet), br. s (broad singlet), d (doublet), dd (double doublet), t (triplet), and m (multiplet). The coupling constant (J) is given in Hz. Typical deuterated solvents were utilized as indicated in the individual examples.

LCMS analyses were typically performed using one of the following conditions:
(1) LCMS spectra were taken on an Agilent Technologies 6120B Quadrupole spectrometer. The mobile phase for the LC was acetonitrile (A) with 0.1% formic acid, and water (B) with 0.1% formic acid, and the eluent gradient was from 5-95% A in 6.0 min, 5%-40% A in 6.0 min, 80-100% A in 6.0 min. using a poroshell 120 EC-C18 50 mm×3.0 mm×2.7 μM capillary column; Flow Rate: 0.7 mL/min. Mass spectra (MS) were measured by electrospray ion-mass spectroscopy (ESI). All temperatures are in degrees Celsius (° C.) unless otherwise noted.
(2) LCMS spectra were taken on an Agilent Technologies 1290-6420 Triple Quadrupole spectrometer: The mobile phase for the LC was acetonitrile (A) with 0.05% formic acid, and water (B) with 0.05% formic acid, and the eluent gradient was from 5-95% A in 5.0 min, using a ZORBAX SB-C18 50 mm×2.1 mm×1.8 μM capillary column; Flow Rate: 0.3 mL/min. Mass spectra (MS) were measured by electrospray ion-mass spectroscopy (ESI). All temperatures are in degrees Celsius unless otherwise noted.
(3) LC-MS analysis was performed using an Agilent 6120b single quadrupole mass spectrometer with an Agilent 1260 infinity II chromatography separations module and Agilent 1260 infinity II photodiode array detector controlled by Agilent Chemstation software. The HPLC column used was an Agilent ZORBAX Eclipse XDB-C18 4.6 mm×150 mm×3.5 μM RapidResol column with a mobile phase of water (0.1% formic acid)/MeCN (0.1% formic acid) and a gradient of 5-95% MeCN over 10 minutes at a flow rate of 1 mL/min. Accurate mass data were obtained using a Thermo Fisher extractive plus EMR orbitrap LCMS system. Exact mass values were calculated by ChemCalc.
(4) LCMS spectra were taken on an alliance Waters 2695 coupled to a dual absorbance detector waters 2487 and a waters micro mass ZQ-2000 single quadrupole spectrometer. The mobile phase for the LC was acetonitrile (A) and water (B) with 0.01% formic acid, and the eluent gradient was from 5-100% A in 10.0 minute using a Kromasil 100-5-C18 150 mm×4.6 mm×5 μm column. Mass spectra (MS) were measured by electrospray ion-mass spectroscopy (ESI). All temperatures are in degrees Celsius unless otherwise noted.

Typically, analytical HPLC mass spectrometry conditions were as follows:

LC1: Agilent Technologies 1260 Infinity coupled, Column: poroshell 120 EC-C18 150 mm×4.6 mm×4 μM; Temperature: 40° C.; Eluent: 5:95 v/v acetonitrile/water+0.02% trifluoroacetic acid in 20 min; Flow Rate: 1.2 mL/min; Detection: VWD, 190-600 nm.

LC2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 μM OBD™ 19×250 mm column with a mobile phase of water/MeCN or water (0.1% TFA)/MeCN (0.1% TFA).

Preparative HPLC were carried out with one of the following two conditions:

Condition 1: GILSON Preparative HPLC System; Column: Ultimate XB-C18, 21.2 mm×250 mm, 5 m; Mobile phase: Water with 0.1% trifluoroacetic acid; MeCN with 0.1% trifluoroacetic acid; Method: 15 minutes gradient elution; Initial organic: 10% to 30%; Final organic: 60% to 80%; UV1: 240; UV2: 230; Flow: 15 mLl/min.

Condition 2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 μM OBD™ 19×250 mm column with a mobile phase of water/MeCN or water (0.1% TFA)/MeCN (0.1% TFA).

Compound names were generated with ChemDraw Professional.

The compounds provided herein, including in various forms such as salts, esters, tautomers, prodrugs, zwitterionic forms, stereoisomers, etc., may be prepared according to various methods including those set forth in the following examples.

### Synthetic Example 1: Synthesis of 4-(6-chloro-8-fluoro-4-(piperazin-1-yl)quinazolin-7-yl)-7-fluorobenzo[d]thiazol-2-amine (Compound 1)

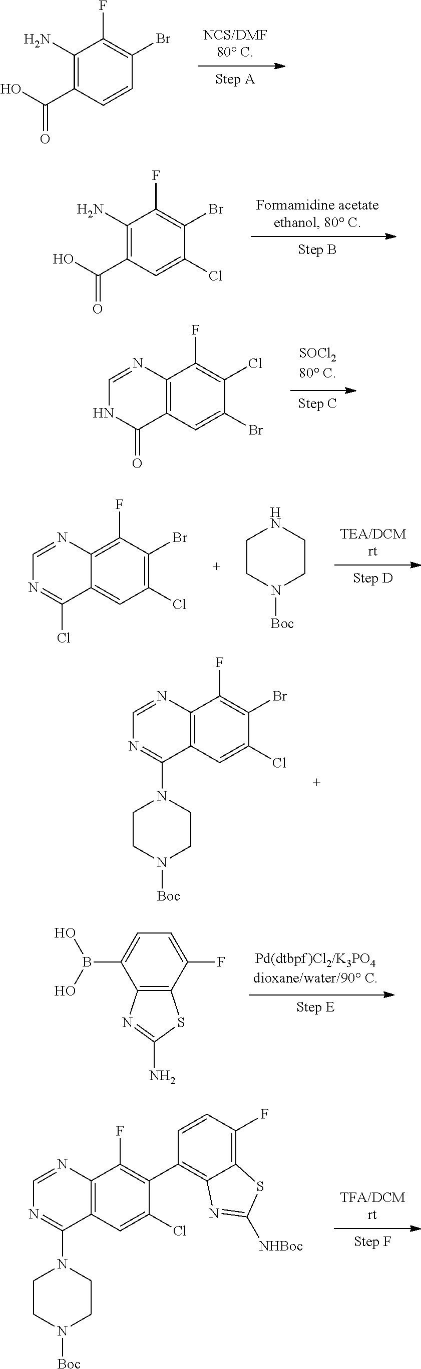

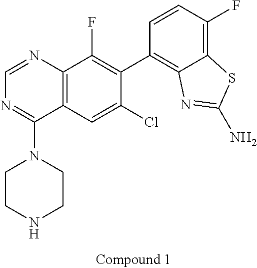 [SMILES: Nc1nc2c(-c3c(Cl)cc4c(N5CCNCC5)ncnc4c3F)ccc(F)c2s1]

Step A: Preparation of 2-amino-4-bromo-5-chloro-3-fluorobenzoic acid: To a solution of 2-amino-4-bromo-3-fluorobenzoic acid (5.0 g, 21.3 mmol) in DMF (50 mL) was added NCS (3.4 g, 25.6 mmol) at ambient temperature. The mixture was warmed to 80° C. and stirred at 80° C. overnight. After cooling to ambient temperature, the mixture was diluted with water (200 mL), solid formed was collected by filtration, washed with water and dried to give 2-amino-4-bromo-5-chloro-3-fluorobenzoic acid (4.69 g, 82%). LCMS ESI (+) m/z 267 (M+H).

Step B: Preparation of 7-bromo-6-chloro-8-fluoroquinazolin-4(3H)-one: To a solution of 2-amino-4-bromo-5-chloro-3-fluorobenzoic acid (4.0 g, 15.0 mmol) in EtOH (40 mL) was added formamidine acetate (15.5 g 149 mmol) at ambient temperature. The mixture was warmed to 80° C. and stirred at 80° C. for 36 hours. After cooling to ambient temperature, the mixture was diluted with ethyl acetate, wash with brine aqueous solution, dried over sodium sulfate, filtered, and concentrated under reduced pressure. The residue was purified by flash column chromatography on silica gel (50:1 DCM/MeOH) to give 7-bromo-6-chloro-8-fluoroquinazolin-4(3H)-one (1.45 g, 35%). LCMS ESI (+) m/z 277 (M+H).

Step C: Preparation of 7-bromo-4,6-dichloro-8-fluoroquinazoline: To a solution 7-bromo-6-chloro-8-fluoroquinazolin-4(3H)-one (600 mg, 2.2 mmol) in SOCl₂ (20 mL) was added DMF (0.2 mL) at ambient temperature. The mixture was warmed to 80° C. and stirred at 80° C. for 3 hours. After cooling to ambient temperature, the reaction solution is directly concentrated under reduced pressure and purified by flash column chromatography on silica gel (10:1 petroleum ether/EtOAc) to give 7-bromo-4,6-dichloro-8-fluoroquinazoline (450 mg, 70%).

Step D: Preparation of tert-butyl 4-(7-bromo-6-chloro-8-fluoroquinazolin-4-yl)piperazine-1-carboxylate: To a solution 7-bromo-4,6-dichloro-8-fluoroquinazoline (100 mg, 0.34 mmol) in DCM (4.5 mL) was added tert-butyl piperazine-1-carboxylate (94.8 mg 0.51 mmol) and triethyl amine (103 mg 1.0 mmol) at ambient temperature. The mixture was warmed to 35° C. and stirred at 35° C. for 4 hours. After cooling to ambient, the mixture was diluted with ethyl acetate, wash with brine aqueous solution, dried over sodium sulfate, filtered, and concentrated under reduced pressure. The residue was purified by flash column chromatography on silica gel (3:1 petroleum ether/EtOAc) to give tert-butyl 4-(7-bromo-6-chloro-8-fluoroquinazolin-4-yl)piperazine-1-carboxylate (120 mg, 79%). LCMS ESI (+) m/z 445 (M+H).

Step E: Preparation of tert-butyl 4-(7-(2-((tert-butoxycarbonyl)amino)-7-fluorobenzo[d]thiazol-4-yl)-6-chloro-8-fluoroquinazolin-4-yl)piperazine-1-carboxylate: To a solution of tert-butyl 4-(7-bromo-6-chloro-8-fluoroquinazolin-4-yl)piperazine-1-carboxylate (120 mg, 0.27 mmol) in dioxane/H₂O (10:3) was added (2-((tert-butoxycarbonyl)amino)-7-fluorobenzo[d]thiazol-4-yl)boronic acid (92.4 mg 0.29 mmol), K₃PO₄ (85.8 mg 0.40 mmol) and Pd(dtbpf)Cl₂(10% mol). The mixture was stirred at 90° C. for 2 hours. After cooling to ambient temperature, the mixture was poured into water and the product was extracted with ethyl acetate, washed with brine aqueous solution, dried over sodium sulfate and concentrated under reduced pressure. The residue was purified by preparative-TLC (DCM/MeOH=20/1) to give tert-butyl 4-(7-(2-((tert-butoxycarbonyl)amino)-7-fluorobenzo[d]thiazol-4-yl)-6-chloro-8-fluoroquinazolin-4-yl)piperazine-1-carboxylate (100 mg, 59%). LCMS ESI (+) m/z 632 (M+H).

Step F: Preparation of 4-(6-chloro-8-fluoro-4-(piperazin-1-yl)quinazolin-7-yl)-7-fluorobenzo[d]thiazol-2-amine: To a solution of tert-butyl 4-(7-(2-((tert-butoxycarbonyl)amino)-7-fluorobenzo[d]thiazol-4-yl)-6-chloro-8-fluoroquinazolin-4-yl)piperazine-1-carboxylate (100 mg, 0.158 mmol) in DCM (3 mL) was added TFA (1 mL). After stirring at room temperature for 2 hours, it was concentrated and purified by preparative RP-HPLC to give 4-(6-chloro-8-fluoro-4-(piperazin-1-yl)quinazolin-7-yl)-7-fluorobenzo[d]thiazol-2-amine (6.2 mg, 6%) as bis trifluoroacetic acid salt. LCMS ESI (+) m/z 433 (M+H). ¹HNMR (400 MHz, CD₃OD) δ 8.79 (s, 1H), 8.07 (s, 1H), 7.26 (t, 1H), 7.03 (t, 1H), 4.18 (t, 4H), 3.49 (t, 4H).

### Synthetic Example 2: Synthesis of N¹-(7-(2-amino-7-fluorobenzo[d]thiazol-4-yl)-6-chloro-8-fluoroquinazolin-4-yl)ethane-1,2-diamine (Compound 5)

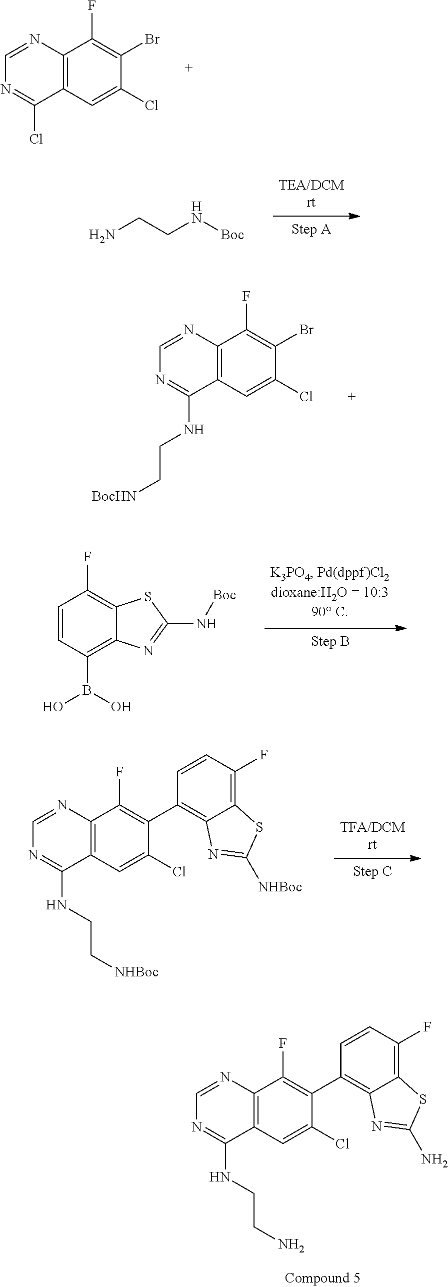 [SMILES: Fc1c(Br)c(Cl)cc2c(Cl)ncnc12 ; CC(C)(C)OC(=O)NCCN ; CC(C)(C)OC(=O)NCCNc1ncnc2c(F)c(Br)c(Cl)cc12 ; CC(C)(C)OC(=O)Nc1nc2c(B(O)O)ccc(F)c2s1 ; CC(C)(C)OC(=O)NCCNc1ncnc2c(F)c(-c3ccc(F)c4sc(NC(=O)OC(C)(C)C)nc34)c(Cl)cc12 ; NCCNc1ncnc2c(F)c(-c3ccc(F)c4sc(N)nc34)c(Cl)cc12]

Step A: Preparation of tert-butyl (2-((7-bromo-6-chloro-8-fluoroquinazolin-4-yl)amino)ethyl)carbamate: 7-bromo-4,6-dichloro-8-fluoroquinazoline (50 mg, 0.17 mmol) was dissolved in DCM (4 mL), then tert-butyl (2-aminoethyl)carbamate (81.6 mg, 0.51 mmol) and TEA (171.8 mg, 1.7 mmol) were added at ambient temperature. The reaction mixture was stirred at 35° C. for 4 hours. The reaction was quenched with water and extracted with ethyl acetate (20 mL×2), the organic layer was separated, washed with water and brine, dried over sodium sulfate, filtered, and concentrated under reduced pressure. The residue was purified by Prep-TLC (EtOAc/hexane:1/1) to give tert-butyl (2-((7-bromo-6-chloro-8-fluoroquinazolin-4-yl)amino)ethyl)carbamate (70 mg, 99%). LCMS: (ES+) m/z 419 ((M+H).

Step B: Preparation of tert-butyl (2-((7-(2-((tert-butoxycarbonyl)-l2-azaneyl)-7-fluorobenzo[d]thiazol-4-yl)-6-chloro-8-fluoroquinazolin-4-yl)amino)ethyl)-l2-azanecarboxylate: To a solution of tert-butyl (2-((7-bromo-6-chloro-8-fluoroquinazolin-4-yl)amino)ethyl)carbamate (70 mg, 0.17 mmol) in 1,4-dioxane (2 mL)/H₂O (0.6 mL) was added (2-((tert-butoxycarbonyl)amino)-7-fluorobenzo[d] thiazol-4-yl)boronic acid (58 mg, 0.19 mmol) and K₃PO₄ (38 mg, 0.18 mmol) under Argon, then Pd(dppf)Cl₂ (12.3 mg, 0.017 mmol) was added, and the reaction mixture was stirred at 90° C. for 4 hours under Argon. After cooling to ambient temperature, the reaction was quenched with water and extracted with EtOAc (20 mL×3), the organic layer was separated, washed with water and brine, dried over sodium sulfate, filtered, and concentrated under reduced pressure. The residue was purified by Preparative-TLC (EtOAc/hexane=3/2) to give tert-butyl (2-((7-(2-((tert-butoxycarbonyl)-l2-azaneyl)-7-fluorobenzo[d]thiazol-4-yl)-6-chloro-8-fluoroquinazolin-4-yl)amino)ethyl)-l2-azanecarboxylate (50 mg, 49%). LCMS ESI (+) m/z 607 (M+H).

Step C: Preparation of N¹-(7-(2-amino-7-fluorobenzo[d]thiazol-4-yl)-6-chloro-8-fluoroquinazolin-4-yl)ethane-1,2-diamine: To a solution of tert-butyl (2-((7-(2-((tert-butoxycarbonyl)-l2-azaneyl)-7-fluorobenzo[d]thiazol-4-yl)-6-chloro-8-fluoroquinazolin-4-yl)amino)ethyl)-l2-azanecarboxylate (50 mg, 0.083 mmol) in DCM (3 mL) was added TFA (1 mL). The reaction mixture was stirred at ambient temperature for 1 hour. The solvent was removed under reduced pressure and the crude product was purified by preparative RPHPLC to give N¹-(7-(2-amino-7-fluorobenzo[d]thiazol-4-yl)-6-chloro-8-fluoroquinazolin-4-yl)ethane-1,2-diamine (21.8 mg, 42%) as bis trifluoroacetic acid salt. LCMS ESI (+) m/z 407 (M+H). ¹HNMR (400 MHz, CD₃OD) δ 8.56 (s, 1H), 8.17 (s, 1H), 7.23 (dd, J=8.4, 5.5 Hz, 1H), 7.01 (t, J=8.8 Hz, 1H), 3.95 (t, J=5.8 Hz, 2H), 3.28 (t, J=5.8 Hz, 2H).

### Synthetic Example 3: Synthesis of N¹-(7-(2-amino-7-fluorobenzo[d]thiazol-4-yl)-6-chloro-8-fluoroquinazolin-4-yl)-N¹-methylethane-1,2-diamine (Compound 19)

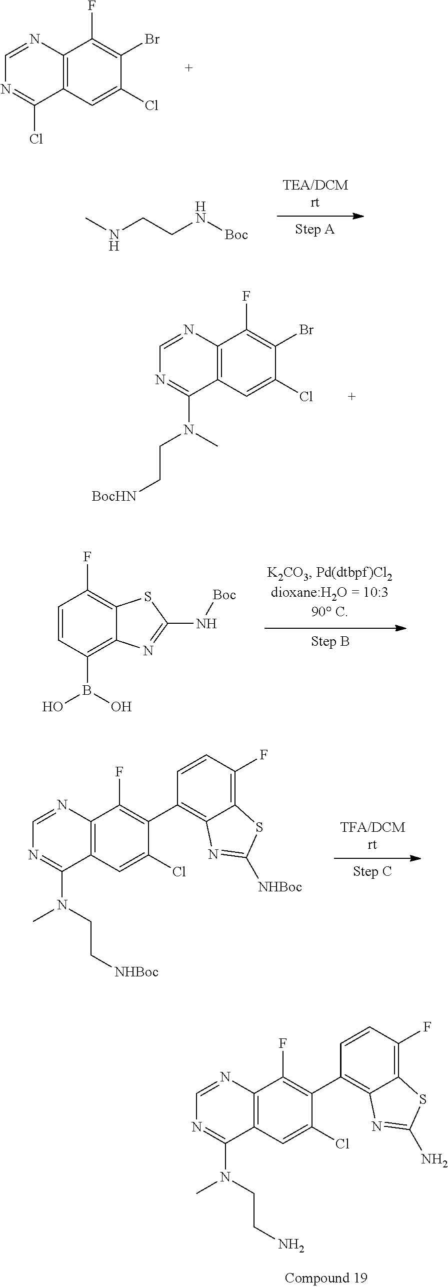 [SMILES: Fc1c(Br)c(Cl)cc2c(Cl)ncnc12 ; CNCCNC(=O)OC(C)(C)C ; CN(CCNC(=O)OC(C)(C)C)c1ncnc2c(F)c(Br)c(Cl)cc12 ; CC(C)(C)OC(=O)Nc1nc2c(B(O)O)ccc(F)c2s1 ; CN(CCNC(=O)OC(C)(C)C)c1ncnc2c(F)c(-c3ccc(F)c4sc(NC(=O)OC(C)(C)C)nc34)c(Cl)cc12 ; CN(CCN)c1ncnc2c(F)c(-c3ccc(F)c4sc(N)nc34)c(Cl)cc12]

Step A: Preparation of tert-butyl (2-((7-bromo-6-chloro-8-fluoroquinazolin-4-yl)(methyl)amino)ethyl)carbamate: To a solution of 7-bromo-4,6-dichloro-8-fluoroquinazoline (50 mg, 0.17 mmol) in DCM (5 mL) was added tert-butyl (2-(methylamino)ethyl)carbamate (35.3 mg, 0.2 mmol) and TEA (34.2 mg, 0.34 mmol). The mixture was stirred at 35° C. for 2 hours. The reaction mixture was quenched with water (20 mL) and the product was extracted with ethyl acetate (20 mL×3). The combined organic layers were washed with brine (150 mL), dried over sodium sulfate, filtered, and concentrated under reduced pressure. The residue was purified by preparative-TLC (petroleum ether/EtOAc: 1/1) to give tert-butyl (2-((7-bromo-6-chloro-8-fluoroquinazolin-4-yl)(methyl)amino)ethyl)carbamate (62 mg, 85%). LCMS (ES+) m/z 433 ((M+H).

Step B: Preparation of tert-butyl (4-(4-((2-((tert-butoxycarbonyl)amino)ethyl)(methyl)amino)-6-chloro-8-fluoroquinazolin-7-yl)-7-fluorobenzo[d]thiazol-2-yl)carbamate: The mixture of tert-butyl (2-((7-bromo-6-chloro-8-fluoroquinazolin-4-yl)(methyl)amino)ethyl)carbamate (62 mg, 0.14 mmol), (2-((tert-butoxycarbonyl)amino)-7-fluorobenzo[d]thiazol-4-yl)boronic acid (53.5 mg, 0.17 mmol), Pd(tbdpf)Cl₂ (16.1 mg, 0.02 mmol), potassium carbonate (75.8 mg, 0.36 mmol) in dioxane (2 mL) and water (0.3 mL) was stirred at 90° C. for 3 hours under argon. After cooling to ambient temperature, the mixture was filtrated through Celite®, washed with ethyl acetate, and concentrated under reduced pressure. The residue was purified by preparative-TLC (DCM:MeOH=10:1) to give tert-butyl (4-(4-((2-((tert-butoxycarbonyl)amino)ethyl)(methyl)amino)-6-chloro-8-fluoroquinazolin-7-yl)-7-fluorobenzo[d]thiazol-2-yl)carbamate (23 mg, 26%). LCMS ESI (+) m/z 621 (M+H).

Step C: Preparation of Ni-(7-(2-amino-7-fluorobenzo[d]thiazol-4-yl)-6-chloro-8-fluoroquinazolin-4-yl)-N¹-methylethane-1,2-diamine: To a solution of tert-butyl (2-((7-(2-((tert-butoxycarbonyl)-l2-azaneyl)-7-fluorobenzo[d]thiazol-4-yl)-6-chloro-8-fluoroquinazolin-4-yl)amino)ethyl)-l2-azanecarboxylate (50 mg, 0.083 mmol) in DCM (3 mL) was added TFA (1 mL). The reaction mixture was stirred at ambient temperature for 1 hour. The solvent was removed under reduced pressure and the crude product was purified by preparative RP-HPLC to give Ni-(7-(2-amino-7-fluorobenzo[d]thiazol-4-yl)-6-chloro-8-fluoroquinazolin-4-yl)-N¹-methylethane-1,2-diamine (21.8 mg, 42%) as bis trifluoroacetic acid salt. LCMS ESI (+) m/z 421 (M+H). ¹HNMR (400 MHz, CD₃OD) δ 8.76 (s, 1H), 8.32 (s, 1H), 7.25 (s, 1H), 7.09 (t, J=8.4 Hz 1H), 2.65 (s, 3H), 2.51-2.55 (m, 4H).

### Synthetic Example 4: Synthesis of 4-(4-(azetidin-3-ylamino)-6-chloro-8-fluoroquinazolin-7-yl)-7-fluorobenzo[d]thiazol-2-amine (Compound 22)

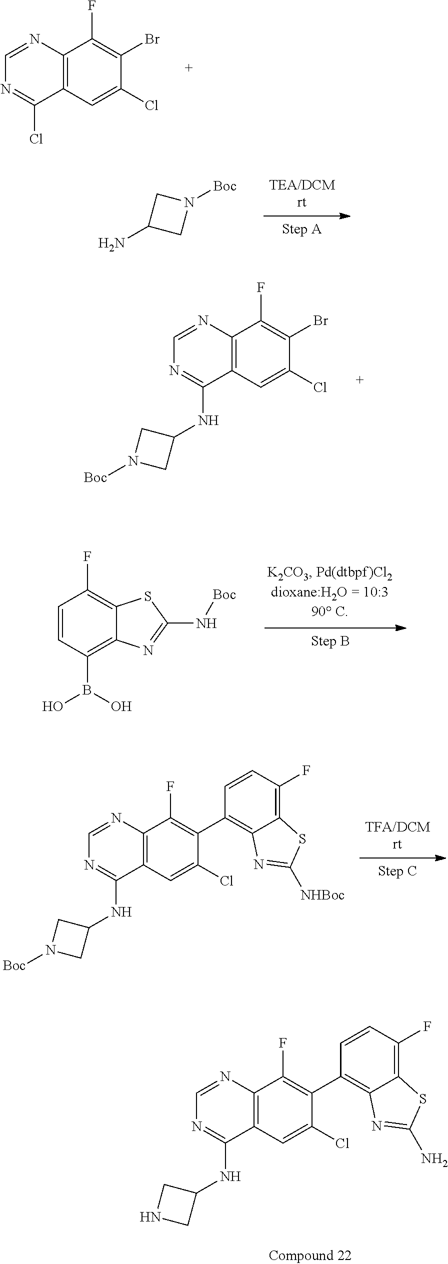 [SMILES: Fc1c(Br)c(Cl)cc2c(Cl)ncnc12 ; CC(C)(C)OC(=O)N1CC(N)C1 ; CC(C)(C)OC(=O)N1CC(Nc2ncnc3c(F)c(Br)c(Cl)cc23)C1 ; CC(C)(C)OC(=O)Nc1nc2c(B(O)O)ccc(F)c2s1 ; CC(C)(C)OC(=O)Nc1nc2c(-c3c(Cl)cc4c(NC5CN(C(=O)OC(C)(C)C)C5)ncnc4c3F)ccc(F)c2s1 ; Nc1nc2c(-c3c(Cl)cc4c(NC5CNC5)ncnc4c3F)ccc(F)c2s1]

Step A: Preparation of tert-butyl 3-((7-bromo-6-chloro-8-fluoroquinazolin-4-yl)amino)azetidine-1-carboxylate: To a solution of 7-bromo-4,6-dichloro-8-fluoroquinazoline (50 mg, 0.17 mmol) in DCM (5 mL) was added tert-butyl 3-aminoazetidine-1-carboxylate (35.1 mg, 0.2 mmol) and TEA (34.2 mg, 0.34 mmol). The mixture was stirred at ambient temperatures for 2 hours. The reaction mixture was quenched with water (20 mL) and extracted with EtOAc (3×20 mL). The combined organic layers were washed with brine (150 mL), dried over sodium sulfate, filtered, and concentrated under reduced pressure, The residue was purified by flash column chromatography on silica gel (petroleum ether/EtOAc=4:1 to 1:1) to give tert-butyl 3-((7-bromo-6-chloro-8-fluoroquinazolin-4-yl)amino)azetidine-1-carboxylate (66 mg, 90%).

Step B: Preparation of tert-butyl 3-((7-(2-((tert-butoxycarbonyl)amino)-7-fluorobenzo[d]thiazol-4-yl)-6-chloro-8-fluoroquinazolin-4-yl)amino)azetidine-1-carboxylate: The mixture of tert-butyl 3-((7-bromo-6-chloro-8-fluoroquinazolin-4-yl)amino)azetidine-1-carboxylate (66 mg, 0.15 mmol), (2-((tert-butoxycarbonyl)amino)-7-fluorobenzo[d]thiazol-4-yl)boronic acid (53.5 mg, 0.17 mmol), Pd(tbdpf)Cl₂ (16.1 mg, 0.02 mmol), potassium carbonate (75.8 mg, 0.36 mmol) in dioxane (2 mL) and water (0.3 mL) was stirred at 90° C. for 3 hours under argon. After cooling to ambient temperature, the mixture was filtrated through Celite®, and washed with ethyl acetate. The filtrate was concentrated under reduced pressure. The residue was purified with preparative-TLC (DCM:MeOH=10:1) to give tert-butyl 3-((7-(2-((tert-butoxycarbonyl)amino)-7-fluorobenzo[d]thiazol-4-yl)-6-chloro-8-fluoroquinazolin-4-yl)amino)azetidine-1-carboxylate (28 mg, 29%).

Step C: Preparation of 4-(4-(azetidin-3-ylamino)-6-chloro-8-fluoroquinazolin-7-yl)-7-fluorobenzo[d]thiazol-2-amine: To a stirred solution of tert-butyl 3-((7-(2-((tert-butoxycarbonyl)amino)-7-fluorobenzo[d]thiazol-4-yl)-6-chloro-8-fluoroquinazolin-4-yl)amino)azetidine-1-carboxylate (28 mg, 0.037 mmol) in DCM (3 mL) was added TFA (1 mL) at 0° C. The mixture was stirred at 0° C. for 2 hours. The mixture was concentrated under reduced pressure and purified by preparative RP-HPLC to give 4-(4-(azetidin-3-ylamino)-6-chloro-8-fluoroquinazolin-7-yl)-7-fluorobenzo[d]thiazol-2-amine (9.2 mg, 31%) as bis trifluoroacetic acid salt. LCMS ESI (+) m/z 419 (M+H). ¹HNMR (400 MHz, CD₃OD) δ 8.59 (s, 1H), 8.28 (s, 1H), 7.26 (t, J=8.4 Hz, 1H), 7.02 (t, J=8.8 Hz, 1H), 5.10-5.13 (m, 4H), 4.31-4.38 (m, 1H).

### Synthetic Example 5: Synthesis of N¹-(7-(2-amino-7-fluorobenzo[d]thiazol-4-yl)-6-chloro-2-(3-(dimethylamino)azetidin-1-yl)-8-fluoroquinazolin-4-yl)ethane-1,2-diamine (Compound 46)

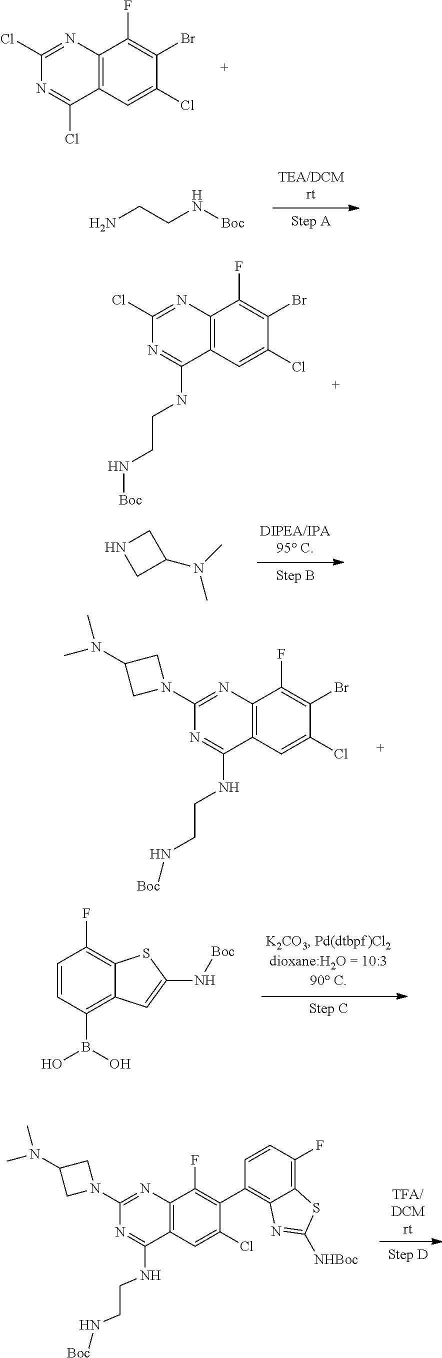

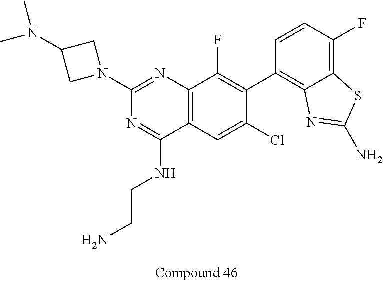 [SMILES: CN(C)C1CN(c2nc(NCCN)c3cc(Cl)c(-c4ccc(F)c5sc(N)nc45)c(F)c3n2)C1]

Step A Preparation of tert-butyl (2-((7-bromo-2,6-dichloro-8-fluoroquinazolin-4-yl)amino)ethyl)carbamate: To a solution of 7-bromo-2,4,6-trichloro-8-fluoroquinazoline (360 mg, 1.09 mmol) in DCM (6 mL) was added TEA (330 mg, 3.27 mmol) and tert-butyl (2-aminoethyl)carbamate (175 mg, 1.09 mmol). The resulting solution was stirred at ambient temperature for 1.5 hours. The reaction was quenched with water (20 mL), and the mixture was extracted with DCM (30 mL×2). The combined organic layers were washed with saturated sodium bicarbonate aqueous solution. The organic phase was dried over anhydrous sodium sulfate, filtered and concentrated under reduced pressure to give tert-butyl (2-((7-bromo-2,6-dichloro-8-fluoroquinazolin-4-yl)amino)ethyl)carbamate (470 mg, 95%) as solid. LCMS ESI (+) m/z 453 (M+H).

Step B: Preparation of tert-butyl (2-((7-bromo-6-chloro-2-(3-(dimethylamino)azetidin-1-yl)-8-fluoroquinazolin-4-yl)amino)ethyl)carbamate: To a suspension of tert-butyl (2-((7-bromo-2,6-dichloro-8-fluoroquinazolin-4-yl) amino)ethyl)carbamate (110 mg, 0.24 mmol) in isopropyl alcohol (6 mL) was added DIPEA (155 mg, 1.2 mmol) and N,N-dimethylazetidin-3-amine dihydrochloride (50 mg, 0.29 mmol). The resulting solution was stirred at 95° C. for 16 hours. After cooling to ambient temperature, solvent was removed under reduced pressure. Water was added and extracted with ethyl acetate (30 mL×2). The combined organic layers were washed with brine aqueous solution. The organic phase was dried over anhydrous sodium sulfate and concentrated under reduced pressure to give tert-butyl (2-((7-bromo-6-chloro-2-(3-(dimethylamino)azetidin-1-yl)-8-fluoroquinazolin-4-yl)amino)ethyl)carbamate (125 mg, 99%) as a solid. LCMS ESI (+) m/z 517 (M+H).

Step C: Preparation of tert-butyl (2-((7-(2-((tert-butoxycarbonyl)amino)-7-fluorobenzo[d]thiazol-4-yl)-6-chloro-2-(3-(dimethylamino)azetidin-1-yl)-8-fluoroquinazolin-4-yl)amino)ethyl)carbamate: To a suspension of tert-butyl (2-((7-bromo-6-chloro-2-(3-(dimethylamino)azetidin-1-yl)-8-fluoroquinazolin-4-yl)amino)ethyl)carbamate (70 mg, 0.13 mmol) in dioxane/H₂O (1.5 mL/0.5 mL) were added (2-((tert-butoxycarbonyl)amino)-7-fluorobenzo[d]thiazol-4-yl)boronic acid (51 mg, 0.16 mmol), K₃PO₄ (41 mg, 0.2 mmol) and Pd(dtppf)Cl₂ (8.5 mg, 0.013 mmol). The resulting solution was stirred at 90° C. for 2 hours under nitrogen. After cooling to ambient temperature, the reaction was quenched with water (20 mL), and the mixture was extracted with DCM (30 mL×2). The combined organic was washed with brine aqueous solution, dried over anhydrous sodium sulfate, filtered, and concentrated under reduced pressure. The residue obtained was purified by preparative-TLC (EtOAc) to give tert-butyl (2-((7-(2-((tert-butoxycarbonyl)amino)-7-fluorobenzo[d]thiazol-4-yl)-6-chloro-2-(3-(dimethylamino)azetidin-1-yl)-8-fluoroquinazolin-4-yl)amino)ethyl)carbamate (75 mg, 78%). LCMS ESI (+) m/z 705 (M+H).

Step D: Preparation of Ni-(7-(2-amino-7-fluorobenzo[d]thiazol-4-yl)-6-chloro-2-(3-(dimethylamino)azetidin-1-yl)-8-fluoroquinazolin-4-yl)ethane-1,2-diamine: To a suspension of tert-butyl (2-((7-(2-((tert-butoxycarbonyl)amino)-7-fluorobenzo[d]thiazol-4-yl)-6-chloro-2-(3-(dimethylamino)azetidin-1-yl)-8-fluoroquinazolin-4-yl)amino)ethyl)carbamate (70 mg, 0.1 mmol) in DCM (6 mL) was added TFA (2 mL). The resulting solution was stirred at ambient temperature for 1 hour. Solvent was removed under reduced pressure, the residue obtained was purified by preparative RP-HPLC to give Ni-(7-(2-amino-7-fluorobenzo[d]thiazol-4-yl)-6-chloro-2-(3-(dimethylamino)azetidin-1-yl)-8-fluoroquinazolin-4-yl)ethane-1,2-diamine (24.2 mg, 28%). LCMS ESI (+) m/z 505 (M+H). ¹HNMR (400 MHz, CD₃OD) 8.16 (s, 1H), 7.21-7.25 (m, 1H), 7.02 (t, J=8.8 Hz, 1H), 4.80-4.85 (m, 2H), 4.60-4.80 (m, 3H), 3.90-4.10 (m, 2H), 3.58 (s, 6H), 3.35-3.42 (m, 2H).

### Synthetic Example 6: Synthesis of 7-(2-amino-7-fluorobenzo[d]thiazol-4-yl)-6-chloro-4-(piperazin-1-yl)quinazoline-8-carbonitrile (Compound 50)

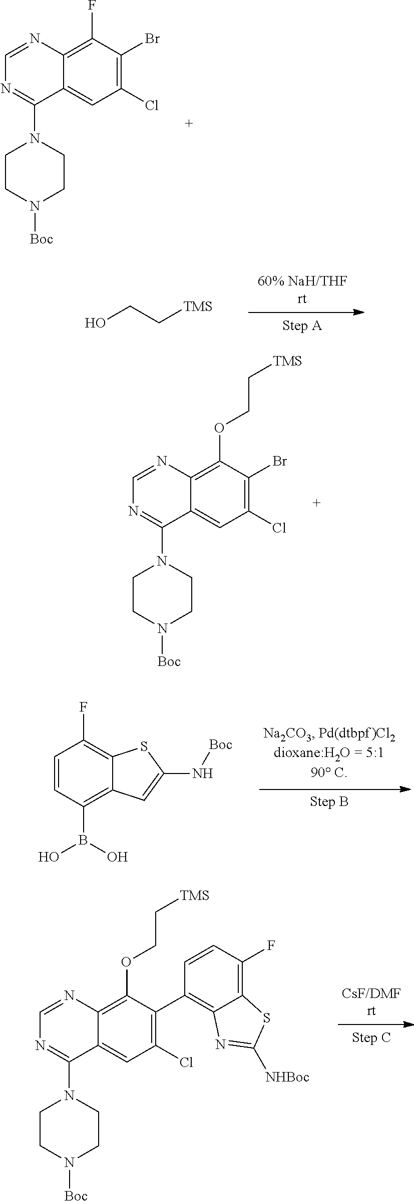 [SMILES: CC(C)(C)OC(=O)N1CCN(c2ncnc3c(F)c(Br)c(Cl)cc23)CC1 ; C[Si](C)(C)CCO ; CC(C)(C)OC(=O)N1CCN(c2ncnc3c(OCC[Si](C)(C)C)c(Br)c(Cl)cc23)CC1 ; CC(C)(C)OC(=O)Nc1cc2c(B(O)O)ccc(F)c2s1 ; CC(C)(C)OC(=O)Nc1nc2c(-c3c(Cl)cc4c(N5CCN(C(=O)OC(C)(C)C)CC5)ncnc4c3OCC[Si](C)(C)C)ccc(F)c2s1]

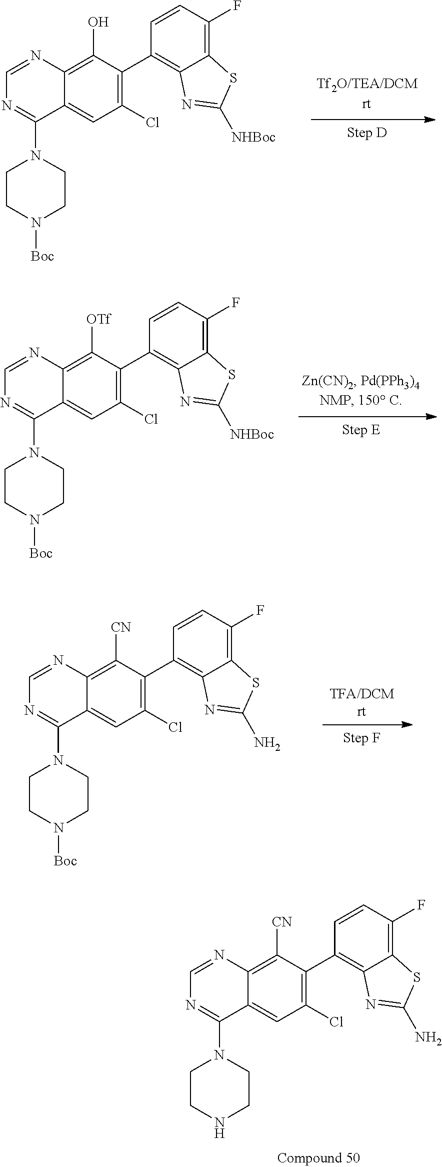 [SMILES: CC(C)(C)OC(=O)Nc1nc2c(-c3c(Cl)cc4c(N5CCN(C(=O)OC(C)(C)C)CC5)ncnc4c3O)ccc(F)c2s1 ; CC(C)(C)OC(=O)Nc1nc2c(-c3c(Cl)cc4c(N5CCN(C(=O)OC(C)(C)C)CC5)ncnc4c3OS(=O)(=O)C(F)(F)F)ccc(F)c2s1 ; CC(C)(C)OC(=O)N1CCN(c2ncnc3c(C#N)c(-c4ccc(F)c5sc(N)nc45)c(Cl)cc23)CC1 ; N#Cc1c(-c2ccc(F)c3sc(N)nc23)c(Cl)cc2c(N3CCNCC3)ncnc12]

Step A: Preparation of tert-butyl 4-(7-bromo-6-chloro-8-(2-(trimethylsilyl)ethoxy)quinazolin-4-yl)piperazine-1-carboxylate: To a solution of 2-(trimethylsilyl)ethan-1-ol (1.83 g, 15.5 mmol) in THE (30 mL) was added 60% NaH (415 mg, 10.4 mmol) at 0° C. The mixture was stirred at 0° C. for 30 minutes. Then tert-butyl 4-(7-bromo-6-chloro-8-fluoroquinazolin-4-yl)piperazine-1-carboxylate (2.3 g, 5.2 mmol) in THE (10 mL) was added to above mixture at ambient temperature and stirred at ambient temperature for 3 hours. Saturated ammonium chloride aqueous solution was added, and the mixture was extracted with ethyl acetate, dried over anhydrous sodium sulfate, filtered, and concentrated under reduced pressure. The residue obtained was purified by flash column chromatography on silica gel (2:1 petroleum ether/ethyl acetate) to give tert-butyl 4-(7-bromo-6-chloro-8-(2-(trimethylsilyl)ethoxy)quinazolin-4-yl)piperazine-1-carboxylate (2.1 g, 75%). LCMS ESI (+) m/z 543 (M+H).

Step B: Preparation of tert-butyl 4-(7-(2-((tert-butoxycarbonyl)amino)-7-fluorobenzo[d]thiazol-4-yl)-6-chloro-8-(2-(trimethylsilyl)ethoxy)quinazolin-4-yl)piperazine-1-carboxylate: To a stirred solution of tert-butyl 4-(7-bromo-6-chloro-8-(2-(trimethylsilyl)ethoxy)quinazolin-4-yl)piperazine-1-carboxylate (1.7 g, 3.1 mmol) in dioxane/H₂O (20 mL/4 mL) was added (2-((tert-butoxycarbonyl)amino)-7-fluorobenzo[d]thiazol-4-yl)boronic acid (1.2 g, 3.8 mmol), Pd(dtbpf)Cl₂ (305 mg, 0.47 mmol) and Na₂CO₃ (829 mg, 0.25 mmol). The mixture was heated to 85° C. and stirred at this temperature for 2 hours. After cooling to ambient temperature, the mixture was diluted with ethyl acetate and the mixture was washed with water (50 mL) and brine (50 mL), dried over sodium sulfate, filtered, and concentrated under reduced pressure. The residue obtained was purified by flash column chromatography on silica gel (1:1 petroleum ether/ethyl acetate) to give tert-butyl 4-(7-(2-((tert-butoxycarbonyl)amino)-7-fluorobenzo[d]thiazol-4-yl)-6-chloro-8-(2-(trimethylsilyl)ethoxy)quinazolin-4-yl)piperazine-1-carboxylate (1.4 g, 61%).

Step C: Preparation of tert-butyl 4-(7-(2-((tert-butoxycarbonyl)amino)-7-fluorobenzo[d]thiazol-4-yl)-6-chloro-8-hydroxyquinazolin-4-yl)piperazine-1-carboxylate: To a solution of tert-butyl 4-(7-(2-((tert-butoxycarbonyl)amino)-7-fluorobenzo[d]thiazol-4-yl)-6-chloro-8-(2-(trimethylsilyl)ethoxy)quinazolin-4-yl)piperazine-1-carboxylate (1.4 g 1.9 mmol) in DMF (10 mL) was added cesium fluoride (874 mg, 5.7 mmol) at ambient temperature and stirred at ambient temperature overnight. Water was added, and the mixture was extracted with ethyl acetate, dried over anhydrous sodium sulfate, filtered, and concentrated under reduced pressure. The residue obtained was purified by flash column chromatography on silica gel (1:1 petroleum ether) to give tert-butyl 4-(7-(2-((tert-butoxycarbonyl)amino)-7-fluorobenzo[d]thiazol-4-yl)-6-chloro-8-hydroxyquinazolin-4-yl)piperazine-1-carboxylate (950 mg, 79%). LCMS ESI (+) m/z 631 (M+H).

Step D: Preparation of tert-butyl 4-(7-(2-((tert-butoxycarbonyl)amino)-7-fluorobenzo[d]thiazol-4-yl)-6-chloro-8-(((trifluoromethyl)sulfonyl)oxy)quinazolin-4-yl)piperazine-1-carboxylate: To a solution of tert-butyl 4-(7-(2-((tert-butoxycarbonyl)amino)-7-fluorobenzo[d]thiazol-4-yl)-6-chloro-8-hydroxyquinazolin-4-yl)piperazine-1-carboxylate (950 mg, 1.5 mmol) and triethyl amine (456 mg, 4.5 mmol) in DCM (20 mL) was added trific anhydride (510 mg, 1.8 mmol) dropwise at 0° C. After addition, the mixture was warmed to ambient temperature and stirred at ambient temperature for 1 hour. The reaction was diluted with water (20 mL) and the mixture was extracted with DCM (20 mL×3), the combined organic layers were washed with brine, dried over sodium sulfate, concentrated under reduced pressure. The residue obtained was purified by flash column chromatography on silica gel (2:1 petroleum ether/ethyl acetate) to give tert-butyl 4-(7-(2-((tert-butoxycarbonyl)amino)-7-fluorobenzo[d]thiazol-4-yl)-6-chloro-8-(((trifluoromethyl)sulfonyl)oxy)quinazolin-4-yl)piperazine-1-carboxylate (730 mg, 64%).

Step E: Preparation of tert-butyl 4-(7-(2-amino-7-fluorobenzo[d]thiazol-4-yl)-6-chloro-8-cyanoquinazolin-4-yl)piperazine-1-carboxylate: To a solution of tert-butyl 4-(7-(2-((tert-butoxycarbonyl)amino)-7-fluorobenzo[d]thiazol-4-yl)-6-chloro-8-(((trifluoromethyl)sulfonyl)oxy)quinazolin-4-yl)piperazine-1-carboxylate (50 mg, 0.065 mmol) in NMP (5 mL) were added zinc cyanide (12 mg, 0.098 mmol) and tetrakis(triphenylphosphine)palladium(0) (75 mg, 0.065 mmol) at ambient temperature. The reaction mixture was heated to 150° C. and stirred at this temperature for 2 hours. After cooling to ambient temperature, water (30 mL) and EtOAc (40 mL) were added. The organic layer was separated, washed with brine, dried over anhydrous sodium sulfate, filtered, and concentrated under reduced pressure. The residue obtained was purified by preparative-TLC (ethyl acetate) to give tert-butyl 4-(7-(2-amino-7-fluorobenzo[d]thiazol-4-yl)-6-chloro-8-cyanoquinazolin-4-yl)piperazine-1-carboxylate (12 mg, 34%). LCMS ESI (+) m/z 540 (M+H).

Step F: Preparation of 7-(2-amino-7-fluorobenzo[d]thiazol-4-yl)-6-chloro-4-(piperazin-1-yl)quinazoline-8-carbonitrile: To a solution of tert-butyl 4-(7-(2-amino-7-fluorobenzo[d]thiazol-4-yl)-6-chloro-8-cyanoquinazolin-4-yl)piperazine-1-carboxylate (12 mg, 0.022 mmol) in DCM (2 mL) was added TFA (1 mL) at 0° C. The mixture was stirred at 0° C. for 2 hours. The mixture was concentrated under reduced pressure and the residue obtained was purified by preparative RP-HPLC to give 7-(2-amino-7-fluorobenzo[d]thiazol-4-yl)-6-chloro-4-(piperazin-1-yl)quinazoline-8-carbonitrile (6.28 mg, 65%) as trifluoroacetic acid salt. LCMS ESI (+) m/z 440 (M+H). ¹HNMR (400 MHz, CD₃OD) δ 8.83 (s, 1H), 8.45 (s, 1H), 7.28-7.32 (dd, J=8.2, 5.2 Hz, 1H), 7.03-7.07 (t, J=8.8 Hz, 1H), 4.12-4.14 (m, 4H), 3.46-3.49 (m, 4H).

### Synthetic Example 7: Synthesis of 4-(4-(azetidin-3-ylamino)-6-chloro-8-fluoro-2-(((S)-1-methylpyrrolidin-2-yl)methoxy)quinazolin-7-yl)-7-fluorobenzo[d]thiazol-2-amine (Compound 55)

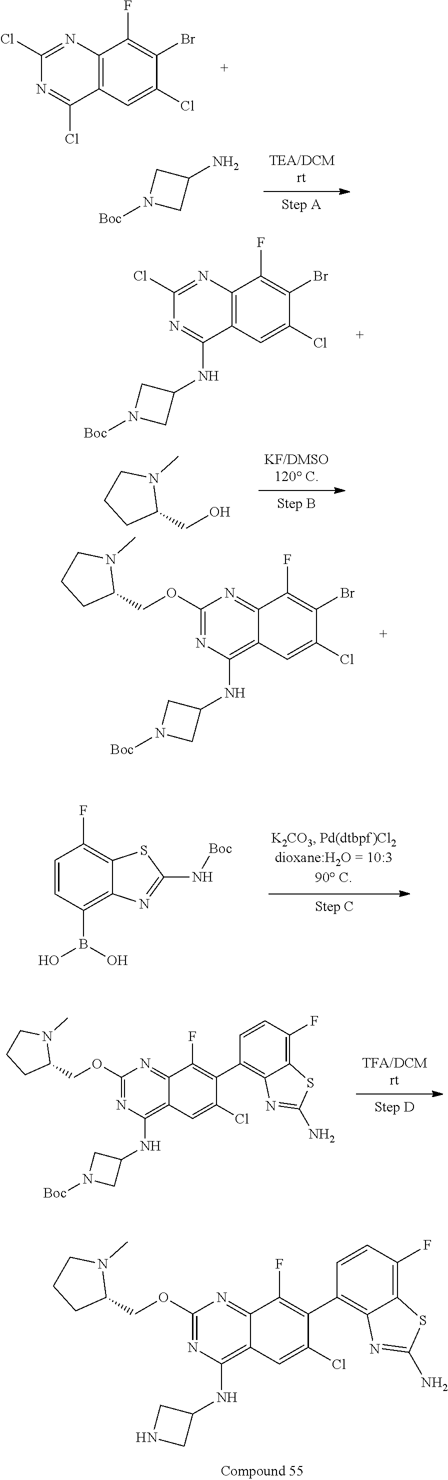

Step A: Preparation of tert-butyl 3-((7-bromo-2,6-dichloro-8-fluoroquinazolin-4-yl)amino)azetidine-1-carboxylate: To a solution of 7-bromo-2,4,6-trichloro-8-fluoroquinazoline (300 mg, 0.91 mmol) in DCM (50 mL) were added triethyl amine (276 mg, 2.72 mmol) and tert-butyl 3-aminoazetidine-1-carboxylate (188 mg, 1.09 mmol) at ambient temperature and the mixture was stirred at ambient temperature overnight. Water was added and the mixture was extracted with ethyl acetate (40 mL×3). The combined organic layers were washed with brine, dried over sodium sulfate, filtered, and concentrated under reduced pressure. The residue obtained was purified by flash column chromatography on silica gel (3:1 petroleum ether/ethyl acetate) to give tert-butyl 3-((7-bromo-2,6-dichloro-8-fluoroquinazolin-4-yl)amino)azetidine-1-carboxylate (310 mg, 73%). LCMS ESI (+) m/z 465 (M+H).

Step B: Preparation of tert-butyl (S)-3-((7-bromo-6-chloro-8-fluoro-2-((1-methylpyrrolidin-2-yl)methoxy)quinazolin-4-yl)amino)azetidine-1-carboxylate: A solution of 3-((7-bromo-2,6-dichloro-8-fluoroquinazolin-4-yl)amino)azetidine-1-carboxylate (320 mg, 0.69 mmol), (S)-(1-methylpyrrolidin-2-yl)methanol (237 mg, 2.06 mol) and potassium fluoride (319 mg, 5.49 mmol) in DMSO (6 mL) was stirred at 118° C. for 6 hours under nitrogen atmosphere. After cooling to ambient temperature, it was diluted with water (40 mL) and extracted with ethyl acetate (80 mL). The organic phase was isolated, dried over sodium sulfate, filtered, and concentrated under reduced pressure. The residue obtained was purified by flash column chromatography on silica gel (50:1 DCM/Methanol) to give the product tert-butyl (S)-3-((7-bromo-6-chloro-8-fluoro-2-((1-methylpyrrolidin-2-yl)methoxy)quinazolin-4-yl)amino)azetidine-1-carboxylate (102 mg, 27%). LCMS ESI (+) m/z 544 (M+H).

Step C: Preparation of tert-butyl 3-((7-(2-((tert-butoxycarbonyl)amino)-7-fluorobenzo[d]thiazol-4-yl)-6-chloro-8-fluoro-2-(((S)-1-methylpyrrolidin-2-yl)methoxy)quinazolin-4-yl)amino)azetidine-1-carboxylate: To a solution of tert-butyl (S)-3-((7-bromo-6-chloro-8-fluoro-2-((1-methylpyrrolidin-2-yl)methoxy)quinazolin-4-yl)amino)azetidine-1-carboxylate (70 mg, 0.10 mmol) in 1,4-dioxane/H₂O (10 mL/3 mL) was added potassium phosphate tribasic (41 mg, 0.19 mmol), (2-((tert-butoxycarbonyl)amino)-7-fluorobenzo[d]thiazol-4-yl)boronic acid (39 mg, 0.13 mmol) and dichloro[1,1′-bis(di-t-butylphosphino)ferrocene]palladium(II) (6 mg, 0.01 mmol) at ambient temperature. The mixture was warmed to 95° C. and stirred at this temperature for 2 hours. After cooling to ambient temperature, the reaction mixture was concentrated under reduced pressure. It was diluted with water (20 mL) and extracted with ethyl acetate (40 mL). The organic phase was dried over sodium sulfate, filtered, and concentrated under reduced pressure. The residue obtained was purified by preparative-TLC (50:1 DCM/Methanol) to give tert-butyl 3-((7-(2-((tert-butoxycarbonyl)amino)-7-fluorobenzo[d]thiazol-4-yl)-6-chloro-8-fluoro-2-(((S)-1-methylpyrrolidin-2-yl)methoxy)quinazolin-4-yl)amino)azetidine-1-carboxylate (35 mg, 48%). LCMS ESI (+) m/z 731 (M+H).

Step C: Preparation of tert-butyl 3-((7-(2-((tert-butoxycarbonyl)amino)-7-fluorobenzo[d]thiazol-4-yl)-6-chloro-8-fluoro-2-(((S)-1-methylpyrrolidin-2-yl)methoxy)quinazolin-4-yl)amino)azetidine-1-carboxylate: To a solution of tert-butyl (S)-3-((7-bromo-6-chloro-8-fluoro-2-((1-methylpyrrolidin-2-yl)methoxy)quinazolin-4-yl)amino)azetidine-1-carboxylate (70 mg, 0.10 mmol) in 1,4-dioxane/H₂O (10 mL/3 mL) were added potassium phosphate (41 mg, 0.19 mmol), (2-((tert-butoxycarbonyl)amino)-7-fluorobenzo[d]thiazol-4-yl)boronic acid (39 mg, 0.13 mmol) and dichloro[1,1′-bis(di-t-butylphosphino)ferrocene]palladium(II) (6 mg, 0.01 mmol) at ambient temperature. The mixture was warmed to 95° C. and stirred at this temperature for 2 hours. After cooling to ambient temperature, the reaction mixture was concentrated under reduced pressure. It was diluted with water (20 mL) and extracted with ethyl acetate (40 mL). The organic phase was dried over sodium sulfate, filtered, and concentrated under reduced pressure. The residue obtained was purified by preparative-TLC (50:1 DCM/Methanol) to give tert-butyl 3-((7-(2-((tert-butoxycarbonyl)amino)-7-fluorobenzo[d]thiazol-4-yl)-6-chloro-8-fluoro-2-(((S)-1-methylpyrrolidin-2-yl)methoxy)quinazolin-4-yl)amino)azetidine-1-carboxylate (35 mg, 48%). LCMS ESI (+) m/z 731 (M+H).

Step D: Preparation of 4-(4-(azetidin-3-ylamino)-6-chloro-8-fluoro-2-(((S)-1-methylpyrrolidin-2-yl)methoxy)quinazolin-7-yl)-7-fluorobenzo[d]thiazol-2-amine: To a solution of 3-((7-(2-((tert-butoxycarbonyl)amino)-7-fluorobenzo[d]thiazol-4-yl)-6-chloro-8-fluoro-2-(((S)-1-methylpyrrolidin-2-yl)methoxy)quinazolin-4-yl)amino)azetidine-1-carboxylate (15 mg, 0.02 mmol) in DCM (3 mL) was added TFA (1 mL) and the mixture was stirred at ambient temperature for 1 hour. The mixture was concentrated under reduced pressure. The residue was purified by preparative RP-HPLC to give 4-(4-(azetidin-3-ylamino)-6-chloro-8-fluoro-2-(((S)-1-methylpyrrolidin-2-yl)methoxy)quinazolin-7-yl)-7-fluorobenzo[d]thiazol-2-amine (5 mg, 28%) as trifluoroacetic acid salt. LCMS ESI (+) m/z 532 (M+H). ¹HNMR (400 MHz, CD₃OD) δ 8.21 (s, 1H), 7.19-7.23 (m, 1H), 7.21-7.24 (m, 1H), 8.80 (t, J=8.8 Hz), 5.15-5.18 (m, 1H), 4.77-4.80 (m, 1H), 4.58-4.63 (m, 1H), 4.44-4.48 (m, 2H), 4.34-4.36 (m, 2H), 3.59-3.68 (m, 2H), 3.078 (m, 1H), 2.98 (s, 3H), 2.33-2.35 (m, 1H), 2.02-2.12 (m, 3H).

### Synthetic Example 8: Synthesis of 7-fluoro-4-(8-fluoro-4-(piperazin-1-yl)-6-(trifluoromethyl)quinazolin-7-yl)benzo[d]thiazol-2-amine (Compound 65)

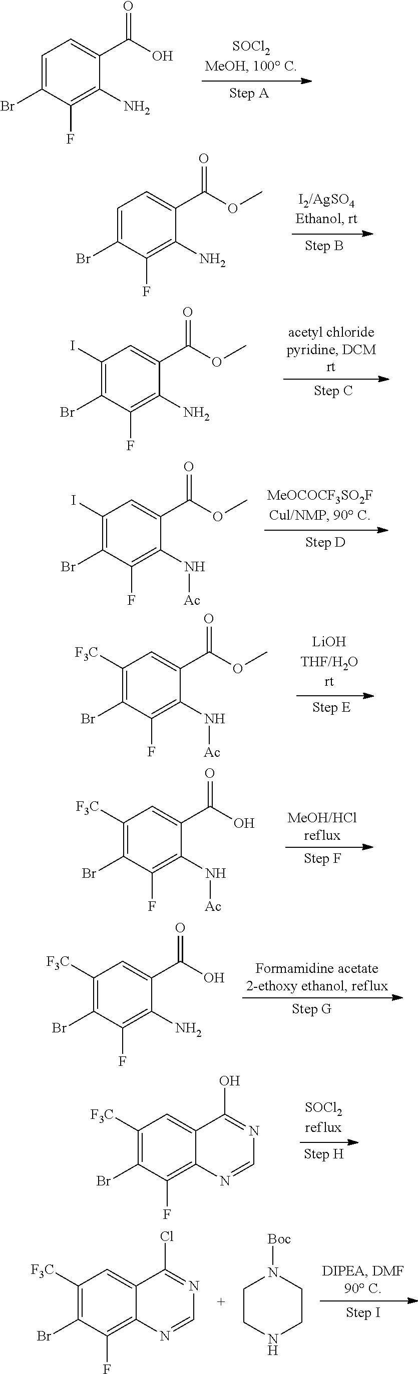

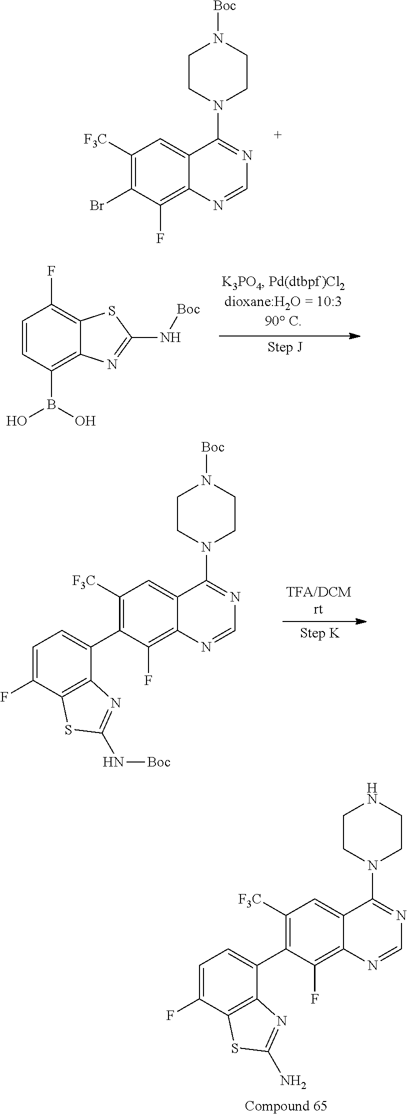 [SMILES: CC(C)(C)OC(=O)N1CCN(c2ncnc3c(F)c(Br)c(C(F)(F)F)cc23)CC1 ; CC(C)(C)OC(=O)Nc1nc2c(B(O)O)ccc(F)c2s1 ; CC(C)(C)OC(=O)Nc1nc2c(-c3c(C(F)(F)F)cc4c(N5CCN(C(=O)OC(C)(C)C)CC5)ncnc4c3F)ccc(F)c2s1 ; Nc1nc2c(-c3c(C(F)(F)F)cc4c(N5CCNCC5)ncnc4c3F)ccc(F)c2s1]

Step A: Preparation of methyl 2-amino-4-bromo-3-fluorobenzoate: To stirring solution of 2-amino-4-bromo-3-fluorobenzoic acid (5.0 g, 21.4 mmol) in MeOH (30 mL) was added dropwise thionyl chloride (15.6 mL, 21 mmol) at 0° C. under argon. The resulting mixture was heated to 100° C. for 16 hours. The solvent was evaporated, and the residue was dissolved in ethyl acetate (100 mL). The organic layer was washed with a saturated aqueous NaHCO₃ solution then dried over Na₂SO₄, filtered, and concentrated under vacuum. The resulting crude material was purified by silica gel column chromatography using EtOAc in hexanes (0% to 20%) as eluent to give methyl 2-amino-4-bromo-3-fluorobenzoate (5.0 g, 94%) as a solid. LCMS ESI (+) m/z 249 (M+H). ¹HNMR (300 MHz, CDCl₃) δ 7.53 (dd, J=8.8, 1.8 Hz, 1H), 6.78 (dd, J=8.8, 6.3 Hz, 1H), 5.93 (s, 1H), 3.90 (s, 1H).

Step B: Preparation of methyl 2-amino-4-bromo-3-fluoro-5-iodobenzoate: To a mixture of iodine (7.16 g, 28 mmol) and silver sulfate (5.3 g, 17 mmol) in EtOH (200 mL), methyl 2-amino-4-bromo-3-fluorobenzoate (5.0 g, 20 mmol) was added and the resulting mixture was stirred at ambient temperature for 45 minutes. The solid was filtered off and washed with DCM, and the filtrate was concentrated under vacuum. The residue was dissolved in DCM and washed with 10% sodium thiosulphate solution, brine and the resulting organic solution was dried over Na₂SO₄, filtered and concentrated under vacuum to give methyl 2-amino-4-bromo-3-fluoro-5-iodobenzoate: the title compound (6.66 g, 88%) as a yellow solid. LCMS ESI (+) m/z 373 (M+H). ¹HNMR (300 MHz, CDCl₃) δ 8.14 (d, J=1.9 Hz, 1H), 5.94 (s, 2H), 3.91 (s, 3H).

Step C: Preparation of methyl 2-acetamido-4-bromo-3-fluoro-5-iodobenzoate: The methyl 2-amino-4-bromo-3-fluoro-5-iodobenzoate (3.50 g, 9.4 mmol) and pyridine (2.3 mL, 28 mmol) were dissolved in DCM at 0° C. Acetyl chloride (0.79 ml, 11 mmol) was added and the reaction was warmed to ambient temperature and stirred at this temperature for 16 hours. The reaction mixture was concentrated under vacuum and the residue obtained was purified by silica gel column chromatography using ethyl acetate in hexanes (0% to 30%) as eluent to give methyl 2-acetamido-4-bromo-3-fluoro-5-iodobenzoate (2.7 g, 69%) as solid. LCMS ESI (+) m/z 417 (M+H). ¹HNMR (300 MHz, CDCl₃) δ 8.87 (s, 1H), 8.25 (s, 1H), 3.95 (s, 3H), 2.26 (s, 3H).

Step D: Preparation of methyl 2-acetamido-4-bromo-3-fluoro-5-(trifluoromethyl)benzoate: To a stirred solution of methyl 4-bromo-2-acetamido-3-fluoro-5-iodobenzoate (1.0 g, 2.4 mmol) and methyl fluorosulfonyldifluoroacetate (0.92 g, 0.72 mmol) in NMP (22.0 mL) at ambient temperature, CuI (0.14 g, 0.73 mmol) was added and the resulting mixture was stirred at 80° C. for 16 hours. After cooling to ambient temperature, the mixture was quenched with water and extracted with ethyl acetate. The organic layer was dried over Na₂SO₄, filtered, concentrated and the crude material was purified by silica gel column chromatography using ethyl acetate in hexanes (0% to 20%) as eluent to give methyl 2-acetamido-4-bromo-3-fluoro-5-(trifluoromethyl)benzoate (0.64 g, 74%) as solid. LCMS ESI (+) m/z 358 (M+H). ¹HNMR (300 MHz, CDCl₃) δ 9.23 (s, 1H), 8.10 (s, 1H), 3.93 (s, 3H), 2.28 (s, 3H).

Step E: Preparation of 2-acetamido-4-bromo-3-fluoro-5-(trifluoromethyl)benzoic acid: Methyl 4-bromo-2-acetamido-3-fluoro-5-(trifluoromethyl)benzoate (3.4 g, 9.49 mmol) was dissolved in THE (56 mL) and water (14 mL) at ambient temperature, then LiOH (0.91 g, 38 mmol) was added. The resulting mixture was stirred at 80° C. for 2 hours. The reaction was diluted with water, acidified with 2M HCl to adjust to a pH˜4 and then extracted with ethyl acetate (2×25 mL). The organic layer was washed with brine, dried over Na₂SO₄, filtered and concentrated in vacuum to afford 4-bromo-2-acetamido-3-fluoro-5-(trifluoromethyl)benzoic acid as solid (3.0 g, 92%). ¹HNMR (300 MHz, CD₃OD) δ 8.10 (s, 1H), 8.01 (s, 1H), 2.21 (s, 3H).

Step F: Preparation of 2-amino-4-bromo-3-fluoro-5-(trifluoromethyl)benzoic acid: 4-Bromo-2-acetamido-3-fluoro-5-(trifluoromethyl)benzoic acid (0.50 g, 1.45 mmol) was dissolved in a 3 M solution of HCl in MeOH (0.064 mL, 1.74 mmol) and refluxed at 80° C. for 2 hours. After cooling to ambient temperature, the reaction mixture was concentrated under vacuum to provide 2-amino-4-bromo-3-fluoro-5-(trifluoromethyl)benzoic acid as solid (0.40 g, 91%). LCMS ESI (−) m/z 300 (M−H). ¹HNMR (300 MHz, CD₃OD) δ 7.86 (s, 1H).

Step G: Preparation of 7-bromo-8-fluoro-6-(trifluoromethyl)quinazolin-4-ol: A mixture of 2-amino-4-bromo-3-fluoro-5-(trifluoromethyl)benzoic acid (0.4 g, 1.32 mmol) and formamidine acetate (0.28 g, 2.65 mmol) in 2-ethoxy ethanol (15 mL) was stirred at reflux for 16 hours. After cooling to ambient temperature, the mixture was concentrated in vacuum. Water was added and extracted with DCM. The organic layer was washed with brine, dried over Na₂SO₄, filtered, and concentrated under vacuum. The crude material was purified by silica gel column chromatography and eluted with 10% MeOH in DCM to provide 7-bromo-8-fluoro-6-(trifluoromethyl)quinazolin-4-ol (0.2 g, 48%) as solid. LCMS ESI (+) m/z 311 (M+H). ¹HNMR (300 MHz, CDCl₃) δ =8.45 (s, 1H), 8.30 (s, 1H).

Step H: Preparation of 7-bromo-4-chloro-8-fluoro-6-(trifluoromethyl)quinazoline: 7-Bromo-8-fluoro-6-(trifluoromethyl)quinazolin-4-ol (0.15 g, 0.48 mmol) was dissolved in thionyl chloride (7.5 mL, 103 mmol) and the reaction mixture stirred at refluxed for 1 hour. After cooling to ambient temperature, the crude was concentrated under vacuum to remove excess thionyl chloride and dried to give 7-bromo-4-chloro-8-fluoro-6-(trifluoromethyl)quinazoline (0.15 g, 97%) as solid. ¹HNMR (300 MHz, CDCl₃) δ 9.22 (s, 1H), 8.47 (s, 1H).

Step I: Preparation of tert-butyl 4-(7-bromo-8-fluoro-6-(trifluoromethyl)quinazolin-4-yl)piperazine-1-carboxylate: To a solution of 7-bromo-4-chloro-8-fluoro-6-(trifluoromethyl)quinazoline (1.70 g, 5.16 mmol) and DIPEA (2.7 mL, 15.5 mmol) in DMF (10 ml) at ambient temperature was added N-Boc piperazine (1.92 g, 10.3 mmol). The resulting mixture was stirred at 90° C. for 1 hour. After cooling to ambient temperature, the crude material was diluted with ethyl acetate (50 mL) and washed with water (50 mL). The organic layer was dried over Na₂SO₄, filtered, and concentrated under vacuum. The residue obtained was purified by silica gel column chromatography and eluted with ethyl acetate in hexane (0% to 30%) to provide tert-butyl 4-(7-bromo-8-fluoro-6-(trifluoromethyl)quinazolin-4-yl)piperazine-1-carboxylate (2.0 g, 81%) as solid. LCMS ESI (+) m/z 479 (M+H). ¹HNMR (300 MHz, CDCl₃) δ 8.82 (s, 1H), 8.02 (s, 1H), 3.94-3.81 (m, 4H), 3.70-3.60 (m, 4H), 1.50 (s, 9H).

Step J: Preparation of tert-butyl 4-(7-(2-((tert-butoxycarbonyl)amino)-7-fluorobenzo[d]thiazol-4-yl)-8-fluoro-6-(trifluoromethyl)quinazolin-4-yl)piperazine-1-carboxylate: tert-Butyl 4-[7-bromo-8-fluoro-6-(trifluoromethyl)quinazolin-4-yl]piperazine-1-carboxylate (150 mg, 0.31 mmol), (2-{[(tert-butoxy)carbonyl]amino}-7-fluoro-1,3-benzothiazol-4-yl)boronic acid) (195 mg, 0.63 mmol) and potassium phosphate (21 mg, 0.63 mmol) were dissolved in 1,4-dioxane (1 mL) and water (0.3 mL). The resulting mixture was purge with argon for 10 minutes then charged with [5-(di-tert-butylphosphanyl)cyclopenta-1,3-dien-1-yl][2-(di-tert-butylphosphanyl)cyclopenta-2,4-dien-1-yl]iron dichloropalladium (20 mg, 0.031 mmol). The reaction mixture was stirred at 90° C. for 7 hours. After cooling to ambient temperature, the resulting mixture was diluted with water and filter through a pad of Celite®, and washed with ethyl acetate. The organic layers were dried over Na₂SO₄, filtered and the solvent was evaporated in vacuum. The crude material was purified by silica gel column chromatography and eluted with ethyl acetate in hexane (0% to 30%) to provide tert-butyl 4-(7-(2-((tert-butoxycarbonyl)amino)-7-fluorobenzo[d]thiazol-4-yl)-8-fluoro-6-(trifluoromethyl)quinazolin-4-yl)piperazine-1-carboxylate (50 mg, 24%). LCMS ESI (+) m/z 667 (M+H). ¹HNMR (300 MHz, CDCl₃) δ 8.85 (s, 1H), 8.11 (s, 1H), 8.00 (s, 1H), 7.34-7.28 (m, 1H), 7.10 (t, J=8.5 Hz, 1H), 3.90 (d, J=5.6 Hz, 4H), 3.69 (d, J=6.0 Hz, 4H), 1.52 (d, J=4.7 Hz, 18H).

Step K: Preparation of 7-fluoro-4-(8-fluoro-4-(piperazin-1-yl)-6-(trifluoromethyl)quinazolin-7-yl)benzo[d]thiazol-2-amine: The tert-butyl 4-[7-(2-{[(tert-butoxy)carbonyl]amino}-7-fluoro-1,3-benzothiazol-4-yl)-8-fluoro-6-(trifluoromethyl)quinazolin-4-yl]piperazine-1-carboxylate) (15 mg, 0.015 mmol) was dissolved in a DCM (1 mL) and cooled down to 0° C. TFA (1.0 mL, 13 mmol) was added dropwise under argon and the reaction mixture was warmed up to ambient temperature and stirred for 1 hour. The resulting mixture was concentrated under vacuum and the residue obtained was triturated with chloroform and filtered to provide 7-fluoro-4-(8-fluoro-4-(piperazin-1-yl)-6-(trifluoromethyl)quinazolin-7-yl)benzo[d]thiazol-2-amine (12 mg, 77%) as a TFA salt. LCMS ESI (+) m/z 467 (M+H). ¹HNMR (300 MHz, CD₃OD) δ 8.89 (s, 1H), 8.33 (s, 1H), 7.33-7.26 (m, 1H), 7.07 (t, J=8.8 Hz, 1H), 4.37-4.30 (m, 4H), 3.58-3.47 (m, 4H).

### Synthetic Example 9: Synthesis of 4-(6-ethynyl-8-fluoro-4-(piperazin-1-yl)quinazolin-7-yl)-7-fluorobenzo[d]thiazol-2-amine (Compound 66)

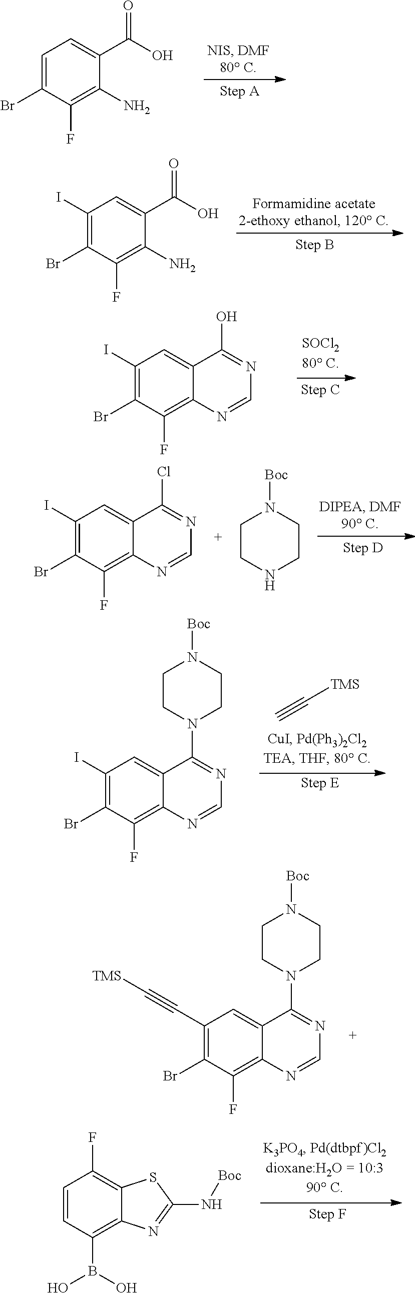

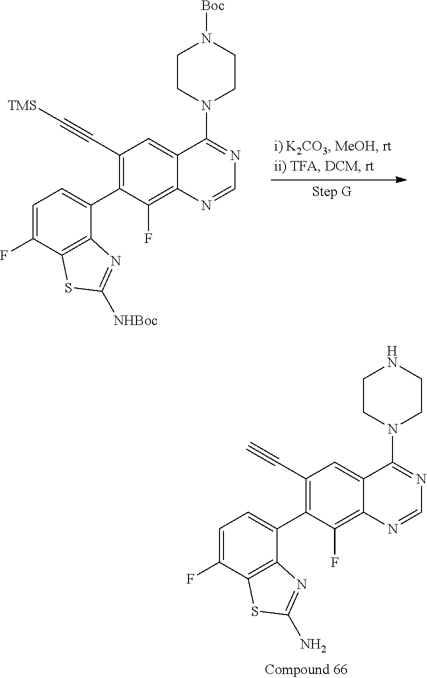 [SMILES: CC(C)(C)OC(=O)Nc1nc2c(-c3c(C#C[Si](C)(C)C)cc4c(N5CCN(C(=O)OC(C)(C)C)CC5)ncnc4c3F)ccc(F)c2s1 ; C#Cc1cc2c(N3CCNCC3)ncnc2c(F)c1-c1ccc(F)c2sc(N)nc12]

Step A: Preparation of 2-amino-4-bromo-3-fluoro-5-iodobenzoic acid: A solution of 2-amino-4-bromo-3-fluorobenzoic acid (1.00 g, 4.3 mmol) and N-iodosuccinimide (1.06 g, 4.7 mmol) in DMF (20.0 mL) was stirred at 80° C. for 2 hours. The resulting solution was diluted with water (100 mL). Solid formed was collected by filtration, washed with water, and dried to afford 2-amino-4-bromo-3-fluoro-5-iodobenzoic acid (1.25 g, 81%). LCMS ESI (−) m/z 358 (M−H). ¹HNMR (300 MHz, CD₃C(O)CD₃) δ 8.16 (d, J=1.9 Hz, 1H), 6.69 (s, 2H).

Step B: Preparation of 7-bromo-8-fluoro-6-iodoquinazolin-4-ol: To a solution of 2-amino-4-bromo-3-fluoro-5-iodobenzoic acid (1.20 g, 3.3 mmol) in ethoxyethanol (24 mL) was added formamidine acetate (1.04 g, 10 mmol) at ambient temperature and then it was stirred at 120° C. for 2 days. After cooling to ambient temperature, water was added, and the mixture was stirred for 5 minutes. The resulting solid was collected by filtration and dried to give 7-bromo-8-fluoro-6-iodoquinazolin-4-ol (1.10 g, 89%). ¹HNMR (300 MHz, CD₃C(O)CD₃) δ 8.51 (d, J=1.7 Hz, 1H), 8.23 (s, 1H).

Step C: Preparation of 7-bromo-4-chloro-8-fluoro-6-iodoquinazoline: To a solution of 7-bromo-8-fluoro-6-iodoquinazolin-4-ol (1.60 g, 4.3 mmol) in thionyl chloride (20.0 mL) was added a few drops of DMF and the resulting solution was heated under reflux for 1 hour. After cooling to ambient temperature, excess thionyl chloride was removed under reduced pressure. The residue was dissolved in ethyl acetate (50 mL) and washed with saturated aq. NaHCO₃ solution. The organic layers were dried over Na₂SO₄ and concentrated in vacuum to give the crude residue which was purified by silica gel column chromatography using ethyl acetate in hexanes (0% to 50%) as eluent to give 7-bromo-4-chloro-8-fluoro-6-iodoquinazoline (0.92 g, 55%). ¹HNMR (300 MHz, CDCl₃) δ 9.12 (s, 1H), 8.68 (s, 1H).

Step D: Preparation of tert-butyl 4-(7-bromo-8-fluoro-6-iodoquinazolin-4-yl)piperazine-1-carboxylate: To a stirred solution of 7-bromo-4-chloro-8-fluoro-6-iodoquinazoline (90.0 mg, 0.232 mmol) in DMF (1.0 mL) was added tert-butyl piperazine-1-carboxylate (43.3 mg, 0.23 mmol) and DIPEA (0.12 mL, 0.697 mmol) at ambient temperature and the resulting mixture was stirred at 90° C. for 1 hour. After cooling to ambient temperature, the crude solution was poured into ice cold water and extracted with ethyl acetate (2×20 mL). The combined organic layers were dried over Na₂SO₄, filtered, concentrated under reduced pressure. The residue obtained was purified on silica gel column chromatography using ethyl acetate in hexane as eluent (0% to 80%) to give tert-butyl 4-(7-bromo-8-fluoro-6-iodoquinazolin-4-yl)piperazine-1-carboxylate (122 mg, 98%). ¹HNMR (300 MHz, CDCl₃) δ 8.76 (s, 1H), 8.23 (d, J=1.9 Hz, 1H), 3.86-3.75 (m, 4H), 3.64 (dd, J=6.4, 3.8 Hz, 4H), 1.50 (s, 9H).

Step E: Preparation of tert-butyl 4-{7-bromo-8-fluoro-6-[2-(trimethylsilyl)ethynyl]quinazolin-4-yl}piperazine-1-carboxylate: To a solution of tert-butyl 4-(7-bromo-8-fluoro-6-iodoquinazolin-4-yl)piperazine-1-carboxylate (48.0 mg, 0.089 mmol) and CuI (2.6 mg, 0.013 mmol) in dry THE (2.2 mL) were added TEA (0.055 mL, 0.398 mmol), ethynyltrimethylsilane (0.063 mL, 0.45 mmol) and Pd(PPh₃)₂Cl₂ (6.30 mg, 0.009 mmol) at ambient temperature under N₂ atmosphere in a sealed tube and degassed for 5 minutes. The resulting reaction mixture was stirred at 80° C. overnight. After cooling to ambient temperature, the solvent was concentrated in vacuum then diluted with water and DCM. The organic layer was separated and washed with brine, dried over Na₂SO₄, filtered and concentrated. The residue was purified on silica gel column chromatography using ethyl acetate in hexane as eluent (0% to 50%) to afford tert-butyl 4-{7-bromo-8-fluoro-6-[2-(trimethylsilyl)ethynyl]quinazolin-4-yl}piperazine-1-carboxylate (30 mg, 66%). LCMS ESI (+) m/z 507 (M+H). ¹HNMR (300 MHz, CDCl₃) δ 8.73 (s, 1H), 7.79 (d, J=1.6 Hz, 1H), 3.89-3.75 (m, 4H), 3.70-3.57 (m, 4H), 1.49 (s, 9H), 0.31 (d, J=1.2 Hz, 9H).

Step F: Preparation of tert-butyl 4-(7-(2-((tert-butoxycarbonyl)amino)-7-fluorobenzo[d]thiazol-4-yl)-8-fluoro-6-((trimethylsilyl)ethynyl)quinazolin-4-yl)piperazine-1-carboxylate: The solution of tert-butyl 4-{7-bromo-8-fluoro-6-[2-(trimethylsilyl)ethynyl]quinazolin-4-yl}piperazine-1-carboxylate (200 mg, 0.39 mmol), (2-{[(tert-butoxy)carbonyl]amino}-7-fluoro-1,3-benzothiazol-4-yl)boronic acid (246 mg, 0.79 mmol) and potassium phosphate (107.3 mg, 0.79 mmol) in 1,4-dioxane (3.0 mL)/water (0.30 mL) was purged with argon for 5 minutes. Then, [5-(di-tert-butylphosphanyl)cyclopenta-1,3-dien-1-yl][2-(di-tert-butylphosphanyl)cyclopenta-2,4-dien-1-yl]iron; dichloropalladium) (25.7 mg, 0.039 mmol) was added. The reaction was stirred at 90° C. for 6 hours. After cooling to ambient temperature, the resulting mixture was filtered through a pad of Celite® and concentrated under reduced pressure to give the crude residue which was purified by silica gel column chromatography using ethyl acetate in hexanes (0% to 80%) as eluent to give tert-butyl 4-(7-(2-((tert-butoxycarbonyl)amino)-7-fluorobenzo[d]thiazol-4-yl)-8-fluoro-6-((trimethylsilyl)ethynyl)quinazolin-4-yl)piperazine-1-carboxylate (150 mg, 55%). LCMS ESI (+) m/z 695 (M+H). ¹HNMR (300 MHz, CDCl₃) δ 8.79 (s, 1H), 7.96 (s, 1H), 7.87 (s, 1H), 7.44 (dd, J=8.4, 5.4 Hz, 1H), 7.10 (t, J=8.6 Hz, 1H), 3.92-3.79 (m, 4H), 3.71-3.62 (m, 4H), 1.54 (s, 9H), 1.51 (s, 9H), −0.04 (s, 9H).

Step G: Preparation of 4-[6-ethynyl-8-fluoro-4-(piperazin-1-yl)quinazolin-7-yl]-7-fluoro-1,3-benzothiazol-2-amine): To a stirred solution of tert-butyl 4-(7-(2-((tert-butoxycarbonyl)amino)-7-fluorobenzo[d]thiazol-4-yl)-8-fluoro-6-((trimethylsilyl)ethynyl)quinazolin-4-yl)piperazine-1-carboxylate (115.4 mg, 0.166 mmol) in MeOH (2.0 mL) was added K₂CO₃ (80.3 mg, 0.581 mmol) and stirred at room temperature for 30 mins. The resulting mixture was evaporated to dryness. To the crude material was added DCM (3.0 mL) and TFA (1.0 mL, 13.1 mmol) and the reaction mixture was stirred for 1 hr. The solvent was evaporated under reduced pressure and purified by reverse phase chromatography (C18-43 g) using CH₃CN in H₂O (5% to 95%) as eluent to give 4-[6-ethynyl-8-fluoro-4-(piperazin-1-yl)quinazolin-7-yl]-7-fluoro-1,3-benzothiazol-2-amine (60.0 mg, 55%). LCMS ESI (+) m/z 423 (M+H). ¹HNMR (300 MHz, CD₃OD) δ 8.75 (s, 1H), 8.09 (s, 1H), 7.30 (dd, J=8.5, 5.4 Hz, 1H), 6.99 (t, J=8.8 Hz, 1H), 4.25-4.07 (m, 4H), 3.57 (s, 1H), 3.54-3.40 (m, 4H).

### Synthetic Example 10: Synthesis of (S)-7-fluoro-4-(8-fluoro-2-((1-methylpyrrolidin-2-yl)methoxy)-4-(piperazin-1-yl)pyrido[4,3-d]pyrimidin-7-yl)benzo[d]thiazol-2-amine (Compound 72)

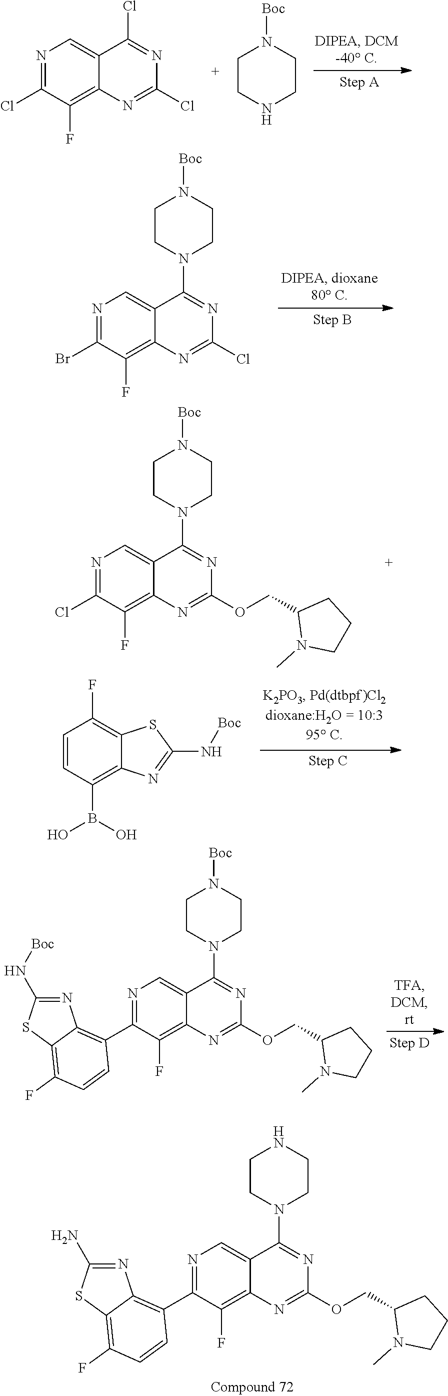

Step A: Preparation of tert-butyl 4-(2,7-dichloro-8-fluoropyrido[4,3-d]pyrimidin-4-yl)piperazine-1-carboxylate: To a mixture of 2,4,7-trichloro-8-fluoropyrido[4,3-d]pyrimidine (WO2020146613) (1.0 g, 4.0 mol) in DCM (20 mL) at −45° C. was added N-ethyl-N-isopropylpropan-2-amine (2.1 mL, 12 mmol) followed by tert-butyl piperazine-1-carboxylate (0.89 g, 4.8 mmol) and stirred at this temperature for 10 minutes. The reaction was quenched with ethyl acetate and saturated aqueous NaHCO₃. The organic layer was separated, dried with Na₂SO₄, filtered, and evaporated under reduce pressure. The residue was purified by flash column chromatography on silica gel with ethyl acetate in hexane (10% to 50%) to provide tert-butyl 4-(2,7-dichloro-8-fluoropyrido[4,3-d]pyrimidin-4-yl)piperazine-1-carboxylate (0.90 g, 56%) as solid. LCMS ESI (+) m/z 402 (M+H). ¹HNMR (300 MHz, CDCl₃) δ 8.90 (s, 1H), 4.27-3.92 (m, 4H), 3.87-3.45 (m, 4H), 1.52 (s, 9H).

Step B: Preparation of (S)-tert-butyl 4-(7-chloro-8-fluoro-2-((1-methylpyrrolidin-2-yl)methoxy)pyrido[4,3-d]pyrimidin-4-yl)piperazine-1-carboxylate: To a mixture of tert-butyl 4-{2,7-dichloro-8-fluoropyrido[4,3-d]pyrimidin-4-yl}piperazine-1-carboxylate (0.80 g, 2.0 mmol) and [(2S)-1-methylpyrrolidin-2-yl]methanol (0.46 g, 4.0 mmol) in p-dioxane (16 mL) was added N-ethyl-N-isopropylpropan-2-amine (1.0 mL, 5.6 mmol) and stirred at 80° C. for 4 hours. After cooled to ambient temperature, solvent was removed under reduced pressure. The residue obtained was purified using silica gel column chromatography with ethyl acetate in MeOH (0% to 10%) to provide (S)-tert-butyl 4-(7-chloro-8-fluoro-2-((1-methylpyrrolidin-2-yl)methoxy)pyrido[4,3-d]pyrimidin-4-yl)piperazine-1-carboxylate (0.48 g, 50%). ¹HNMR (300 MHz, CDCl₃) δ 8.77 (s, 1H), 4.55 (dd, J=10.8, 4.7 Hz, 1H), 4.36 (dd, J=10.8, 6.6 Hz, 1H), 4.05-3.82 (m, 4H), 3.79-3.31 (m, 4H), 3.23-2.82 (m, 1H), 2.74 (m, 1H), 2.51 (s, 3H), 2.31 (dd, J=17.6, 8.5 Hz, 1H), 2.15-2.01 (m, 1H), 1.82 (m, 3H), 1.59 (s, 9H).

Step C: Preparation of (S)-tert-butyl 4-(7-(2-((tert-butoxycarbonyl)amino)-7-fluorobenzo[d]thiazol-4-yl)-8-fluoro-2-((1-methylpyrrolidin-2-yl)methoxy)pyrido[4,3-d]pyrimidin-4-yl)piperazine-1-carboxylate: A mixture of tert-butyl 4-(7-chloro-8-fluoro-2-{[(2S)-1-methylpyrrolidin-2-yl]methoxy}pyrido[4,3-d]pyrimidin-4-yl)piperazine-1-carboxylate (0.15 g, 0.31 mmol), 2-{[(tert-butoxy)carbonyl]amino}-7-fluoro-1,3-benzothiazol-4-yl)boronic acid (0.20 g, 0.624 mmol) and cesium carbonate (0.20 g, 0.62 mmol) in p-dioxane (4.3 mL) and water (0.86 mL) was degassed with argon, followed by the addition of Pd(dppf)Cl₂ (0.046 g, 0.062 mmol), then degassed again. The reaction mixture was stirred at 95° C. for 3 hours then cooled down to ambient temperature. The resulting mixture was diluted with ethyl acetate and brine, and the organic layer was separated. The organic layer was dried over Na₂SO₄, filtered, and concentrated under reduced pressure. The crude was purified using silica gel column chromatography with DCM in MeOH (0% to 10%) to provide (S)-tert-butyl 4-(7-(2-((tert-butoxycarbonyl)amino)-7-fluorobenzo[d]thiazol-4-yl)-8-fluoro-2-((1-methylpyrrolidin-2-yl)methoxy)pyrido[4,3-d]pyrimidin-4-yl)piperazine-1-carboxylate (0.090 g, 40%). LCMS ESI (+) m/z 713 (M+H). ¹HNMR (300 MHz, CDCl₃) δ 9.07 (s, 1H), 7.72 (dd, J=8.6, 5.4 Hz, 1H), 7.15 (t, J=8.7 Hz, 1H), 4.59 (m, 1H), 4.41 (m, 1H), 3.99 (m, 4H), 3.68 (m, 4H), 3.17 (m, 1H), 2.77 (m, 1H), 2.54 (s, 3H), 2.32 (m, 1H), 2.07 (m, 1H), 1.83 (m, 3H), 1.58 (s, 9H), 1.53 (s, 9H).

Step D: Preparation of 7-fluoro-4-(8-fluoro-2-{[(2S)-1-methylpyrrolidin-2-yl]methoxy}-4-(piperazin-1-yl)pyrido[4,3-d]pyrimidin-7-yl)-1,3-benzothiazol-2-amine: To tert-butyl (S)-4-(7-(2-((tert-butoxycarbonyl)amino)-7-fluorobenzo[d]thiazol-4-yl)-8-fluoro-2-((1-methylpyrrolidin-2-yl)methoxy)pyrido[4,3-d]pyrimidin-4-yl)piperazine-1-carboxylate (18 mg, 0.025 mmol) in DCM (2 ml) was added TFA (0.5 mL), and the reaction mixture was stirred at ambient temperature for 3 hours. The resulting mixture was evaporated under reduced pressure. The resulting solid was triturated with ether for 30 minutes and solid was collected and dried to give 7-fluoro-4-(8-fluoro-2-{[(2S)-1-methylpyrrolidin-2-yl]methoxy}-4-(piperazin-1-yl)pyrido[4,3-d]pyrimidin-7-yl)-1,3-benzothiazol-2-amine (0.015 g, 95%) as TFA salt. LCMS ESI (+) m/z 513 (M+H). ¹HNMR (300 MHz, CD₃OD) δ 9.28 (s, 1H), 7.89 (d, J=5.5 Hz, 1H), 7.22 (t, J=8.9 Hz, 1H), 5.08-4.92 (m, 1H), 4.75 (dd, J=13.0, 6.7 Hz, 1H), 4.49-4.22 (m, 4H), 3.93 (m, 1H), 3.73 (m, 1H), 3.65-3.47 (m, 4H), 3.32 (m, 1H), 3.14 (s, 3H), 2.46 (m, 1H), 2.12 (m, 3H).

### Synthetic Example 11: Synthesis of 7-(2-amino-7-fluorobenzo[d]thiazol-4-yl)-8-fluoro-4-(piperazin-1-yl)quinazoline-6-carbonitrile (Compound 94)

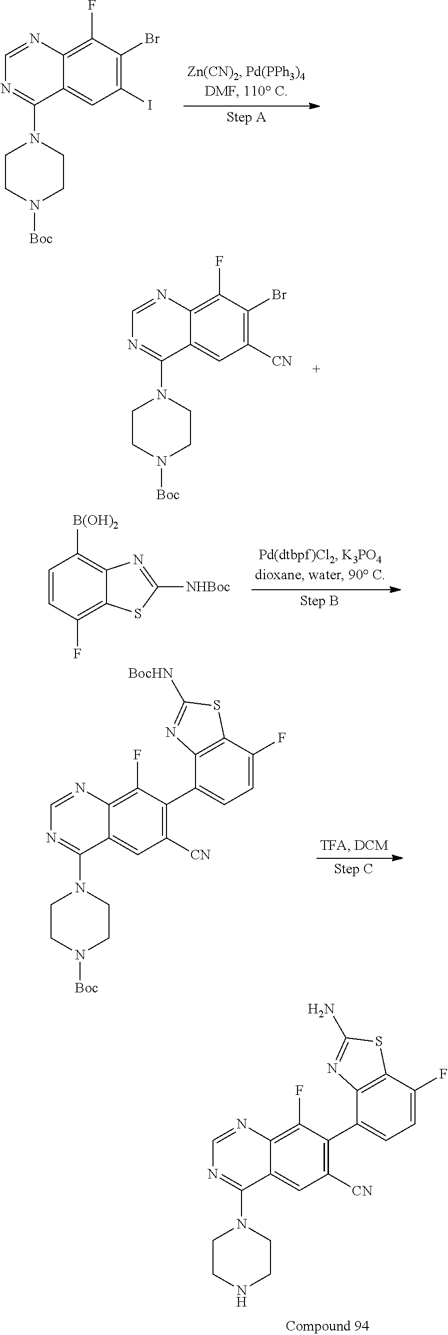 [SMILES: CC(C)(C)OC(=O)N1CCN(c2ncnc3c(F)c(Br)c(I)cc23)CC1 ; CC(C)(C)OC(=O)N1CCN(c2ncnc3c(F)c(Br)c(C#N)cc23)CC1 ; CC(C)(C)OC(=O)Nc1nc2c(B(O)O)ccc(F)c2s1 ; CC(C)(C)OC(=O)Nc1nc2c(-c3c(C#N)cc4c(N5CCN(C(=O)OC(C)(C)C)CC5)ncnc4c3F)ccc(F)c2s1 ; N#Cc1cc2c(N3CCNCC3)ncnc2c(F)c1-c1ccc(F)c2sc(N)nc12]

Step A: Preparation of tert-butyl 4-(7-bromo-6-cyano-8-fluoroquinazolin-4-yl)piperazine-1-carboxylate: A mixture of tert-butyl 4-(7-bromo-8-fluoro-6-iodoquinazolin-4-yl)piperazine-1-carboxylate (200.0 mg, 0.37 mmol) and zinc cyanide (21.9 mg, 0.186 mmol) in DMF (3.0 mL) was purged with nitrogen for 5 minutes. Then was added tetrakis(triphenylphosphine)palladium (21.5 mg, 0.019 mmol), was further purged for additional 5 minutes and stirred at 110° C. for 8 h. The cooled reaction mixture was diluted with EtOAc, washed twice with 10% aqueous LiCl, then with brine. The aqueous layers were extracted with EtOAc. The combined organic layers were dried and concentrated. The crude material was purified by silica gel column chromatography (25 g) using EtOAc in hexanes (0-80%) as eluent to give tert-butyl 4-(7-bromo-6-cyano-8-fluoroquinazolin-4-yl)piperazine-1-carboxylate (120 mg, 74%). LCMS ESI (+) m/z 436 (M+H). ¹H NMR (300 MHz, CDCl₃) δ 8.80 (s, 1H), 8.02 (d, J=1.6 Hz, 1H), 3.97-3.76 (m, 4H), 3.65 (dd, J=6.2, 4.1 Hz, 4H), 1.50 (s, 9H).

Step B: Preparation of tert-butyl 4-(7-(2-((tert-butoxycarbonyl)amino)-7-fluorobenzo[d]thiazol-4-yl)-6-cyano-8-fluoroquinazolin-4-yl)piperazine-1-carboxylate: The solution of tert-butyl 4-(7-bromo-6-cyano-8-fluoroquinazolin-4-yl)piperazine-1-carboxylate (120.0 mg, 0.28 mmol), (2-{[(tert-butoxy)carbonyl]amino}-7-fluoro-1,3-benzothiazol-4-yl)boronic acid (128.8 mg, 0.41 mmol), and potassium phosphate (74.9 mg, 0.550 mmol) in 1,4-dioxane (2.0 mL) and water (0.2 mL) was purged with argon for 5 minutes. Then was added [5-(di-tert-butylphosphanyl)cyclopenta-1,3-dien-1-yl][2-(di-tert-butylphosphanyl)cyclopenta-2,4-dien-1-yl]iron; dichloropalladium (17.9 mg, 0.028 mmol) and was purged an additional 5 minutes. After that the reaction mixture was stirred at 90° C. for 2 hours. After cooling to ambient temperature, the reaction mixture was passed through Celite® and the filtrate was concentrated under reduced pressure to give crude residue which was purified by silica gel column chromatography (24 g) using EtOAc in hexanes (0-80%) as eluent to give tert-butyl 4-[7-(2-{[(tert-butoxy)carbonyl]amino}-7-fluoro-1,3-benzothiazol-4-yl)-6-cyano-8-fluoroquinazolin-4-yl]piperazine-1-carboxylate (140 mg, 82%). LCMS ESI (+) m/z 624 (M+H). ¹H NMR (300 MHz, CDCl₃) δ 8.82 (s, 1H), 8.75 (s, 1H), 8.14 (s, 1H), 7.53 (dd, J=8.3, 5.2 Hz, 1H), 7.16 (t, J=8.6 Hz, 1H), 3.99-3.77 (m, 4H), 3.68-3.54 (m, 4H), 1.50 (s, 9H), 1.47 (s, 9H).

Step C: Preparation of 7-(2-amino-7-fluorobenzo[d]thiazol-4-yl)-8-fluoro-4-(piperazin-1-yl)quinazoline-6-carbonitrile): To a solution of tert-butyl 4-[7-(2-{[(tert-butoxy)carbonyl]amino}-7-fluoro-1,3-benzothiazol-4-yl)-6-cyano-8-fluoroquinazolin-4-yl]piperazine-1-carboxylate (70.0 mg, 0.11 mmol) in DCM (2.0 mL) was added TFA (0.50 mL, 6.53 mmol). After one hour, the reaction was completed and evaporated to dryness to give crude residue, which was purified by reverse phase chromatography (C18-24 g) using acetonitrile in H₂O (5%-95%) as eluent to give 7-(2-amino-7-fluoro-1,3-benzothiazol-4-yl)-8-fluoro-4-(piperazin-1-yl)quinazoline-6-carbonitrile; bis(trifluoroacetic acid) (10.8 mg, 15%). LCMS ESI (+) m/z 424 (M+H). ¹H NMR (300 MHz, CD₃OD) δ 8.80 (s, 1H), 8.41 (s, 1H), 7.38 (dd, J=8.5, 5.4 Hz, 1H), 7.05 (t, J=8.8 Hz, 1H), 4.26-4.05 (m, 4H), 3.51-3.36 (m, 4H).

### Synthetic Example 12: Synthesis of 4-(6-chloro-8-fluoro-5-methoxy-4-(piperazin-1-yl)quinazolin-7-yl)-7-fluorobenzo[d]thiazol-2-amine (Compound 125)

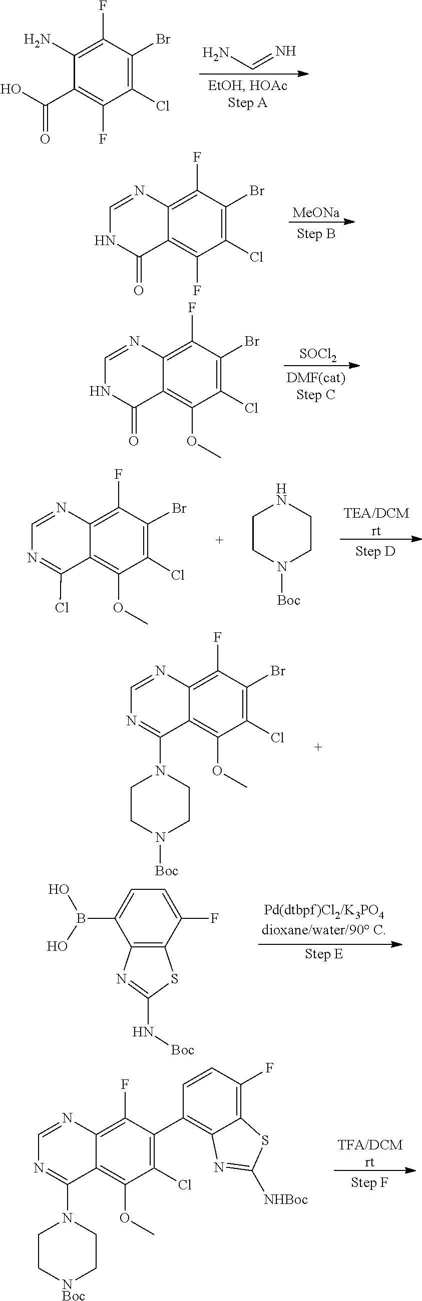

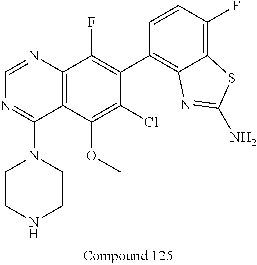 [SMILES: COc1c(Cl)c(-c2ccc(F)c3sc(N)nc23)c(F)c2ncnc(N3CCNCC3)c12]

Step A: Preparation of 7-bromo-6-chloro-5,8-difluoro-3H-quinazolin-4-one: 2-amino-4-bromo-5-chloro-3,6-difluoro-benzoic acid (500 mg, 1.75 mmol) was added to a solution of formamidine acetate (1817 mg, 17.5 mmol) in HOAc (40 mL) and ethanol (40 mL) at ambient temperature. The solution was stirred at 100° C. for 12 hours. After cooling to ambient temperature, the solution was poured into water and the precipitate collected by filtration, washed with water, and dried under vacuum to give 7-bromo-6-chloro-5,8-difluoro-3H-quinazolin-4-one (300 mg, 52%) as solid. LCMS ESI (+) m/z 294.8 (M+H).

Step B: Preparation of 7-bromo-6-chloro-8-fluoro-5-methoxy-3H-quinazolin-4-one: To a solution of 7-bromo-6-chloro-5,8-difluoro-3H-quinazolin-4-one (200 mg, 0.68 mmol) in DMF (5 mL) and methanol (5 mL) was added sodium methanolate (183 mg, 3.38 mmol) at ambient temperature. The mixture was stirred at 80° C. for 2 hours. After cooling to ambient temperature, the reaction mixture was diluted with ethyl acetate, and washed with brine twice. The organic layers were dried (sodium sulfate), filtered, and concentrated at reduced pressure to give 7-bromo-6-chloro-8-fluoro-5-methoxy-3H-quinazolin-4-one (150 mg, 50%) as solid. LCMS ESI (+) m/z 306.8 (M+H).

Step C: Preparation of 7-bromo-4,6-dichloro-8-fluoro-5-methoxy-quinazoline: Thionyl chloride (5.0 mL, 68.5 mmol) was added to 7-bromo-6-chloro-8-fluoro-5-methoxy-3H-quinazolin-4-one (150 mg, 0.49 mmol). The mixture was stirred at 80° C. for 3 hours. After cooling to ambient temperature, the reaction mixture was concentrated under reduced pressure. The residue obtained was diluted with DCM and added to ice water slowly, then adjusted to pH-8 with saturated sodium bicarbonate. The organic layer was separated, dried (sodium sulfate), filtered, and concentrated under reduced pressure to give 7-bromo-4,6-dichloro-8-fluoro-5-methoxy-quinazoline (120 mg, 68%) as solid. LCMS ESI (+) m/z 325.0 (M+H).

Step D: Preparation of tert-butyl4-(7-bromo-6-chloro-8-fluoro-5-methoxy-quinazolin-4-yl)piperazine-1-carboxylate: To a solution of 7-bromo-4,6-dichloro-8-fluoro-5-methoxy-quinazoline (120 mg, 0.37 mmol) in DCM (5 mL) was added triethylamine (0.15 mL, 1.10 mmol) and 1-Boc-piperazine (103 mg, 0.55 mmol). The solution was stirred at ambient temperature for 3 hours. The solution was diluted with DCM, and washed brine. The organic layer was separated, dried (sodium sulfate), filtered, and concentrated under reduced pressure. The residue was purified by preparative-TLC (EtOAc/PE=1/5) to give tert-butyl4-(7-bromo-6-chloro-8-fluoro-5-methoxy-quinazolin-4-yl)piperazine-1-carboxylate (140 mg, 72%) as solid. LCMS ESI (+) m/z 474.9 (M+H).

Step E: Preparation of tert-butyl 4-[7-[2-(tert-butoxycarbonylamino)-7-fluoro-1,3-benzothiazol-4-yl]-6-chloro-8-fluoro-5-methoxy-quinazolin-4-yl]piperazine-1-carboxylate: To a solution of tert-butyl 4-(7-bromo-6-chloro-8-fluoro-5-methoxy-quinazolin-4-yl) piperazine-1-carboxylate (140 mg, 0.29 mmol) and [2-(tert-butoxycarbonyl amino)-7-fluoro-1,3-benzothiazol-4-yl]boronic acid (110 mg, 0.35 mmol) in 1,4-dioxane (5 mL)/water (1 mL) was added 1,1′-bis (di-t-butylphosphino); dichloro[1,1′-bis(di-tert-butylphosphino)ferrocene]palladium(II) (15 mg, 0.024 mmol) and potassium phosphate (125 mg, 0.59 mmol) at ambient temperature. The solution was stirred at 90° C. for 2 hours under N₂. After cooling to ambient temperature, the solution was diluted with ethyl acetate and washed with water twice. The organic phase was separated, dried (sodium sulfate), filtered, and concentrated under reduced pressure. The residue obtained was purified by preparative-TLC (EtOAc/PE=1/4) to give tert-butyl 4-[7-[2-(tert-butoxycarbonylamino)-7-fluoro-1,3-benzothiazol-4-yl]-6-chloro-8-fluoro-5-methoxy-quinazolin-4-yl]piperazine-1-carboxylate (30 mg, 12%) as solid. LCMS ESI (+) m/z 663.3 (M+H).

Step F: Preparation of 4-(6-chloro-8-fluoro-5-methoxy-4-piperazin-1-yl-quinazolin-7-yl)-7-fluoro-1,3-benzothiazol-2-amine: To a solution of tert-butyl 4-[7-[2-(tert-butoxycarbonylamino)-7-fluoro-1,3-benzothiazol-4-yl]-6-chloro-8-fluoro-5-methoxy-quinazolin-4-yl]piperazine-1-carboxylate (31 mg, 0.046 mmol) in DCM (2 mL) was added trifluoroacetic acid (1.0 mL, 13.0 mmol) at ambient temperature and stirred at ambient temperature for 1 hour. The solution was concentrated under reduced pressure. Half of the crude was purified by preparative RP-HPLC to give 4-(6-chloro-8-fluoro-5-methoxy-4-piperazin-1-yl-quinazolin-7-yl)-7-fluoro-1,3-benzothiazol-2-amine (5.2 mg, 24%) as solid. LCMS ESI (+) m/z 463.2 (M+H). ¹HNMR (400 MHz, CD₃OD): δ 8.72 (s, 1H), 7.25-7.28 (m, 1H), 7.02-7.06 (m, 1H), 4.01 (s, 4H), 3.88 (s, 3H), 3.45-3.48 (m, 4H).

### Synthetic Example 13: Synthesis of 7-fluoro-4-(8-fluoro-6-(furan-3-yl)-2-(((S)-1-methylpyrrolidin-2-yl)methoxy)-4-(piperazin-1-yl)quinazolin-7-yl)benzo[d]thiazol-2-amine (Compound 139)

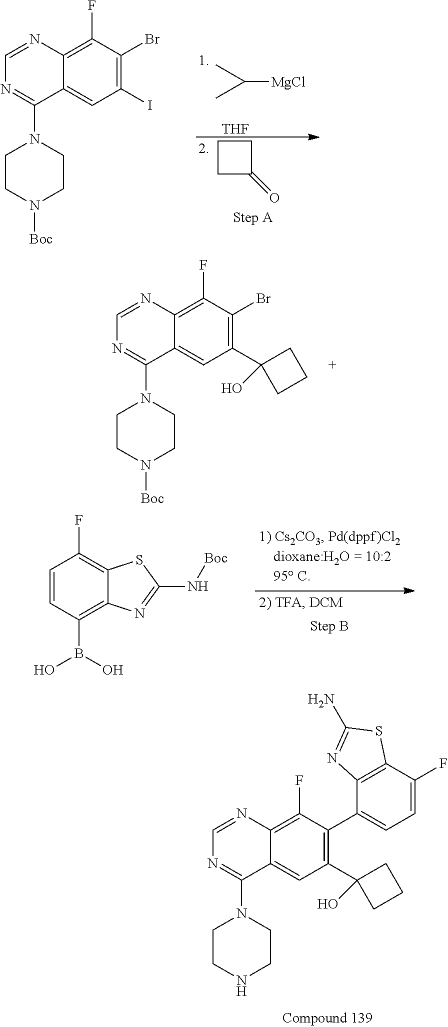

Step A: Preparation of tert-butyl 4-(7-bromo-8-fluoro-6-(1-hydroxycyclobutyl)quinazolin-4-yl)piperazine-1-carboxylate: A suspension of tert-butyl 4-(7-bromo-8-fluoro-6-iodoquinazolin-4-yl)piperazine-1-carboxylate (0.15 g, 0.28 mmol) in THE (2.8 mL) at −78° C. was added a 2 M THE solution of isopropylmagnesium chloride (0.15 mL, 0.29 mmol) and the mixture was kept at −78° C. for 1 hour. To the reaction mixture was added a solution of cyclobutanone (0.023 g, 0.33 mmol) in 200 μL of THF, the mixture was brought slowly to room temperature. The reaction was left at room temperature for 18 hours followed by extraction with EA-NH₄Cl, the EA was collected, dried over sodium sulfate, filtered, and evaporated. The mixture was purified on a 40 g gold column with 10% EA in hexane to 100% EA over 25 min to provide tert-butyl 4-(7-bromo-8-fluoro-6-(1-hydroxycyclobutyl)quinazolin-4-yl)piperazine-1-carboxylate (0.020 g, 15%). LCMS ESI (+) m/z 481.04 (M+H). ¹H NMR (300 MHz, CDCl₃) δ 8.79 (s, 1H), 7.69 (s, 1H), 3.82 (m, 4H), 3.67 (m, 4H), 2.88-2.69 (m, 2H), 2.62 (m, 2H), 2.31 (m, 1H), 1.79 (m, 1H), 1.55 (s, 9H).

Step B: Preparation of 1-(7-(2-amino-7-fluorobenzo[d]thiazol-4-yl)-8-fluoro-4-(piperazin-1-yl)quinazolin-6-yl)cyclobutan-1-ol: To a mixture of tert-butyl 4-(7-bromo-8-fluoro-6-(1-hydroxycyclobutyl)quinazolin-4-yl)piperazine-1-carboxylate (0.020 g, 0.041 mmol), (2-((tert-butoxycarbonyl)amino)-7-fluorobenzo[d]thiazol-4-yl)boronic acid (0.019 g, 0.061 mmol) and cesium carbonate (0.027 g, 0.082 mmol) in 1,4-dioxane (1 mL) and water (0.22 mL) was degassed with argon followed by the addition of Pd(dppf)Cl₂ (0.006 g, 0.008 mmol). The mixture was heated at 95° C. for 2 hrs. The mixture was evaporated and purified on a 25 g gold column with 10% EA in hexane to 100% EA to provide crude intermediate which was taken in DCM (2 mL) and excess of TFA. After 1 hr at room temperature the reaction mixture was purified on preparative RP-HPLC to provide of 1-(7-(2-amino-7-fluorobenzo[d]thiazol-4-yl)-8-fluoro-4-(piperazin-1-yl)quinazolin-6-yl)cyclobutan-1-ol (0.002 g, 10%). LCMS ESI (+) m/z 469.15 (M+H). ¹H NMR (300 MHz, CD₃OD) δ 8.61 (s, 1H), 7.74 (s, 1H), 7.53-7.24 (m, 1H), 6.99 (t, J=8.9 Hz, 1H), 3.94 (m, 4H), 3.06 (m, 4H), 2.50-2.36 (m, 1H), 2.35-2.19 (m, 1H), 2.21-1.96 (m, 2H), 1.88-1.70 (m, 1H), 1.71-1.55 (m, 1H).

### Synthetic Example 14: Synthesis of 7-fluoro-4-(8-fluoro-6-(furan-3-yl)-2-(((S)-1-methylpyrrolidin-2-yl)methoxy)-4-(piperazin-1-yl)quinazolin-7-yl)benzo[d]thiazol-2-amine (Compound 143)

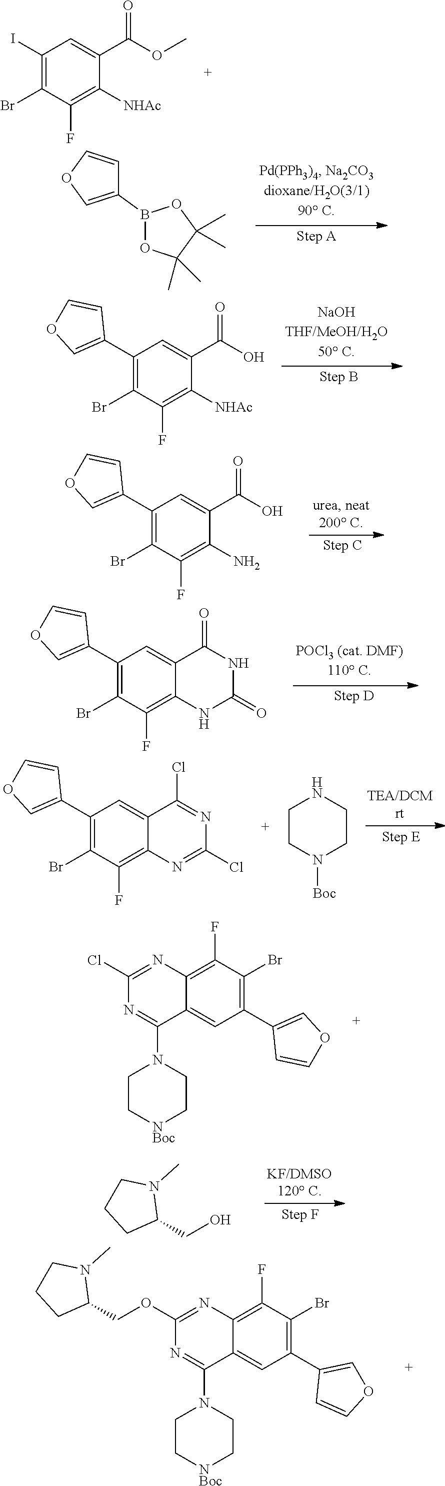

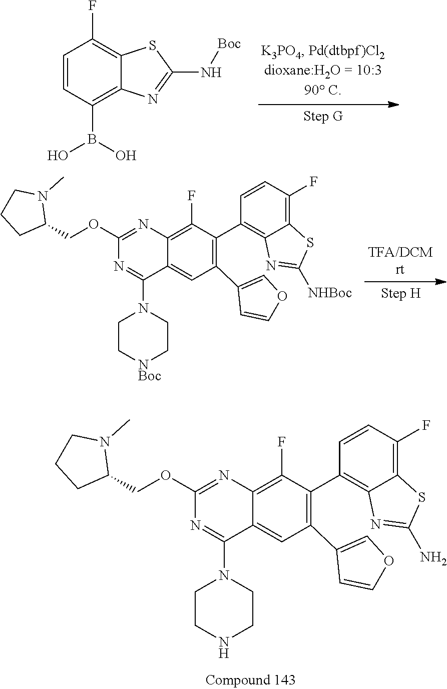

Step A: Preparation of 2-acetamido-4-bromo-3-fluoro-5-(3-furyl)benzoic acid: To methyl 2-acetamido-4-bromo-3-fluoro-5-iodo-benzoate (5.00 g, 12.0 mmol) in 1,4-dioxane (60 mL) and water (15 mL) was added 3-furylboronic acid (1.61 g, 14.4 mmol) at room temperature under Ar. Then Pd(PPh₃)₄ (1.11 g, 0.96 mmol) and Na₂CO₃ (3.82 g, 36.1 mmol) were added under Ar. The mixture was stirred overnight at 90° C. under Ar. After cooling to room temperature, the reaction mixture was diluted with water (50 mL) and EtOAc (50 mL) and then pH was adjusted to 4-5 with saturated aqueous solution of sodium hydrogen sulfate. Organic layer was separated, washed with brine, dried (sodium sulfate), filtered, and was concentrated under reduced pressure. The residue obtained was suspended in ethyl acetate and stirred for 5 minutes. Solid was collected by filtration and dried to give 2-acetamido-4-bromo-3-fluoro-5-(3-furyl)benzoic acid (3.00 g, 73%) as solid. LCMS ESI (+) m/z 342.0 (M+H).

Step B: Preparation of 2-amino-4-bromo-3-fluoro-5-(3-furyl)benzoic acid: To a solution of 2-acetamido-4-bromo-3-fluoro-5-(3-furyl)benzoic acid (3.50 g, 10.2 mmol) in THE (40 mL)/methanol (40 mL)/water (20 mL) was added NaOH (4.09 g, 102 mmol) at ambient temperature. The mixture was stirred at 50° C. for 12 hours. After cooling to ambient temperature, organic layers were removed under reduced pressure. The aqueous layer was acidified with 1 N HCl to pH 3-4. The solid formed was collected by filtration and then dried to give 2-amino-4-bromo-3-fluoro-5-(3-furyl)benzoic acid (2.30 g, 75%). LCMS ESI (+) m/z 300.0 (M+H).

Step C: Preparation of 7-bromo-8-fluoro-6-(3-furyl)-1H-quinazoline-2,4-dione: 2-amino-4-bromo-3-fluoro-5-(3-furyl)benzoic acid (2.30 g, 7.66 mmol) and urea (9.21 g, 153 mmol) were placed in 50 mL round bottom flask and heated to 200° C. after mixed well. The mixture was dissolved and then solidified again. After stirred for 2 hours at 200° C., the mixture was cooled to 100° C. and water (40 mL) was added. The reaction mixture was stirred at 100° C. for 2 hours. The solid was evenly dispersed. After cooling to room temperature, the solid was collected by filtration and dried to give 7-bromo-8-fluoro-6-(3-furyl)-1H-quinazoline-2,4-dione (1.85 g, 74%). LCMS ESI (+) m/z 325.0 (M+H).

Step D: Preparation of 7-bromo-2,4-dichloro-8-fluoro-6-(3-furyl)quinazoline: To 7-bromo-8-fluoro-6-(3-furyl)-1H-quinazoline-2,4-dione (600 mg, 1.85 mmol) in phosphorus oxychloride (8.0 mL, 85.8 mmol) was added 2 drops of DMF. The mixture was stirred at 110° C. for 4 hours. After cooling to ambient temperature, phosphorus oxychloride was removed under reduced pressure. The residue was dissolved in small amount of DCM and TEA (1 mL). The mixture was directly purified by flash chromatography on silica gel eluting with 30% EtOAc in petroleum ether to give 7-bromo-2,4-dichloro-8-fluoro-6-(3-furyl)quinazoline (360 mg, 54%).

Step E: Preparation of tert-butyl 4-[7-bromo-2-chloro-8-fluoro-6-(3-furyl)quinazolin-4-yl]piperazine-1-carboxylate: To a stirred solution of 7-bromo-2,4-dichloro-8-fluoro-6-(3-furyl)quinazoline (500 mg, 1.38 mmol) in DCM (20 mL) was added triethyl amine (0.39 mL, 2.76 mmol) and 1-Boc-piperazine (283 mg, 1.52 mmol) at ambient temperature and stirred at this temperature for 4 hours. The mixture was poured to water. The aqueous phase was separated and extracted twice with ethyl acetate. The combined organic phases were dried over sodium sulfate, filtered and the solvent was evaporated in vacuo. The residue was chromatographed over silica gel eluting with DCM/MeOH=20/1 to afford the tert-butyl 4-[7-bromo-2-chloro-8-fluoro-6-(3-furyl)quinazolin-4-yl]piperazine-1-carboxylate (520 mg, 73%) as solid.

Step F: Preparation of tert-butyl 4-[7-bromo-8-fluoro-6-(3-furyl)-2-[[(2S)-1-methylpyrrolidin-2-yl]methoxy]quinazolin-4-yl]piperazine-1-carboxylate: To a solution of tert-butyl 4-[7-bromo-2-chloro-8-fluoro-6-(3-furyl)quinazolin-4-yl]piperazine-1-carboxylate (200 mg, 0.39 mmol) in DMSO (2 mL) were added (S)-(1-methylpyrrolidin-2-yl)methanol (135 mg, 1.17 mmol) and KF (182 mg, 3.13 mmol) at ambient temperature. The mixture was stirred at 120° C. for 2 hours. After cooling to ambient temperature, water and ethyl acetate were added. The organic layer was separated, washed with brine, dried (sodium sulfate), filtered, and concentrated under reduced pressure. The crude product was purified by preparative-TLC with DCM:MeOH=20:1 to give tert-butyl 4-[7-bromo-8-fluoro-6-(3-furyl)-2-[[(2S)-1-methylpyrrolidin-2-yl]methoxy]quinazolin-4-yl]piperazine-1-carboxylate (123 mg, 53%) as solid. LCMS ESI (+) m/z 590.2 (M+H).

Step G: Preparation of tert-butyl 4-[7-[2-(tert-butoxycarbonylamino)-7-fluoro-1,3-benzothiazol-4-yl]-8-fluoro-6-(3-furyl)-2-[[(2S)-1-methylpyrrolidin-2-yl]methoxy]quinazolin-4-yl]piperazine-1-carboxylate: Tert-butyl 4-[7-bromo-8-fluoro-6-(3-furyl)-2-[[(2S)-1-methylpyrrolidin-2-yl]methoxy]quinazolin-4-yl]piperazine-1-carboxylate (123 mg, 0.21 mmol), [2-(tert-butoxycarbonylamino)-7-fluoro-1,3-benzothiazol-4-yl]boronic acid (85 mg, 0.27 mmol), 1,1′-Bis(di-t-butylphosphino)ferrocene palladium dichloride (14 mg, 0.021 mmol) and potassium phosphate (88 mg, 0.42 mmol) in 1,4-dioxane (2 mL) and water (0.40 mL) was stirred at 90° C. for 1 hour under Ar. After cooling to ambient temperature, water and ethyl acetate were added. The organic layer was separated, washed with brine, dried (sodium sulfate), filtered, and concentrated under reduced pressure. The crude product was purified by Preparative-TLC, eluting with DCM:MeOH=10:1, to give tert-butyl 4-[7-[2-(tert-butoxycarbonylamino)-7-fluoro-1,3-benzothiazol-4-yl]-8-fluoro-6-(3-furyl)-2-[[(2S)-1-methylpyrrolidin-2-yl]methoxy]quinazolin-4-yl]piperazine-1-carboxylate (140 mg, 86%) as solid. LCMS ESI (+) m/z 778.3 (M+H).

Step H: Preparation of 7-fluoro-4-[8-fluoro-6-(3-furyl)-2-[[(2S)-1-methylpyrrolidin-2-yl]methoxy]-4-piperazin-1-yl-quinazolin-7-yl]-1,3-benzothiazol-2-amine: To the solution of tert-butyl 4-[7-[2-(tert-butoxycarbonylamino)-7-fluoro-1,3-benzothiazol-4-yl]-8-fluoro-6-(3-furyl)-2-[[(2S)-1-methylpyrrolidin-2-yl]methoxy]quinazolin-4-yl]piperazine-1-carboxylate (0.14 g, 0.18 mmol) in DCM (3 mL) was added trifluoroacetic acid (1.0 mL, 13.0 mmol) at ambient temperature and stirred at this temperature for 2 hours. The mixture was concentrated to dryness under vacuum and the residue was purified by preparative RP-HPLC to afford 7-fluoro-4-[8-fluoro-6-(3-furyl)-2-[[(2S)-1-methylpyrrolidin-2-yl]methoxy]-4-piperazin-1-yl-quinazolin-7-yl]-1,3-benzothiazol-2-amine (95 mg, 93%) as solid. LCMS ESI (+) m/z 578.1 (M+H). ¹HNMR (400 MHz, CD₃OD) δ 7.88 (s, 1H), 7.33 (s, 1H), 7.18 (s, 1H), 7.07-7.14 (m, 1H), 6.96 (t, J=8.8 Hz, 1H), 6.15 (s, 1H), 4.90-5.00 (m, 2H), 4.66-4.75 (m, 1H), 4.18 (s, 4H), 3.86-3.96 (m, 1H), 3.68-3.80 (m, 1H), 3.45-3.57 (m, 4H), 3.10 (s, 3H), 2.36-2.48 (m, 1H), 2.16-2.27 (m, 1H), 2.03-2.16 (m, 2H).

### Synthetic Example 15: Synthesis of 2-amino-1-(4-(7-(2-amino-7-fluorobenzo[d]thiazol-4-yl)-8-fluoro-6-(furan-3-yl)-2-(((S)-1-methylpyrrolidin-2-yl)methoxy)quinazolin-4-yl)piperazin-1-yl)ethan-1-one (Compound 144)

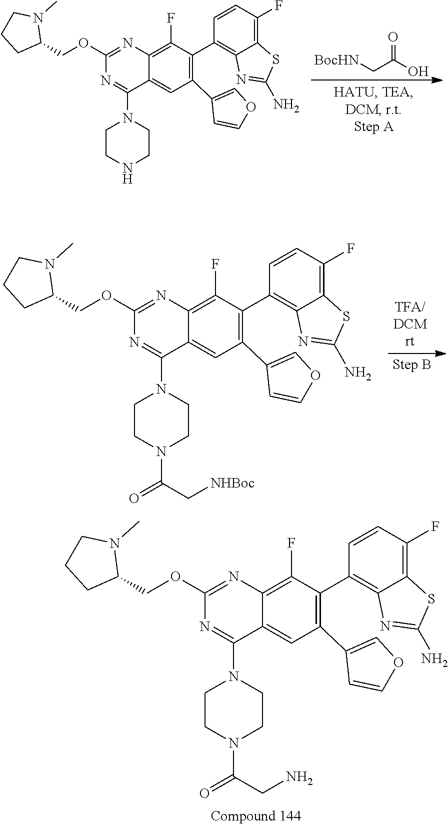

Step A: Preparation of tert-butyl (2-(4-(7-(2-amino-7-fluorobenzo[d]thiazol-4-yl)-8-fluoro-6-(furan-3-yl)-2-(((S)-1-methylpyrrolidin-2-yl)methoxy)quinazolin-4-yl)piperazin-1-yl)-2-oxoethyl)carbamate: A solution of Boc-glycine (11 mg, 0.060 mmol), HATU (58 mg, 0.15 mmol) and trimethyl amine (0.021 mL, 0.15 mmol) in DCM (2 mL) was stirred at ambient temperature for 15 minutes, and a mixture of 7-fluoro-4-(8-fluoro-6-(furan-3-yl)-2-(((S)-1-methylpyrrolidin-2-yl)methoxy)-4-(piperazin-1-yl)quinazolin-7-yl)benzo[d]thiazol-2-amine (30 mg, 0.050 mmol) and triethyl amine (0.021 mL, 0.15 mmol) in DCM (2 mL) was added. The mixture was stirred at ambient temperature for 1 hour. Water and DCM were added. The organic layers were then separated and dried over Na₂SO₄, filtered, and concentrated under reduced pressure to give tert-butyl (2-(4-(7-(2-amino-7-fluorobenzo[d]thiazol-4-yl)-8-fluoro-6-(furan-3-yl)-2-(((S)-1-methylpyrrolidin-2-yl)methoxy)quinazolin-4-yl)piperazin-1-yl)-2-oxoethyl)carbamate (40 mg, 0.0531 mmol, 100%) as solid. LCMS (ES+) m/z 735.2 (M+H).

Step B: Preparation of 2-amino-1-(4-(7-(2-amino-7-fluorobenzo[d]thiazol-4-yl)-8-fluoro-6-(furan-3-yl)-2-(((S)-1-methylpyrrolidin-2-yl)methoxy)quinazolin-4-yl)piperazin-1-yl)ethan-1-one: To the solution of tert-butyl N-[2-[4-[7-(2-amino-7-fluoro-1,3-benzothiazol-4-yl)-8-fluoro-6-(3-furyl)-2-[[(2S)-1-methylpyrrolidin-2-yl]methoxy]quinazolin-4-yl]piperazin-1-yl]-2-oxo-ethyl]carbamate (40 mg, 0.054 mmol) in DCM (3 mL) was added trifluoroacetic acid (1.0 mL, 13.0 mmol) at ambient temperature and stirred for 2 hours at this temperature. The solvent was removed under vacuum and the mixture was purified by preparative RP-HPLC to give 2-amino-1-(4-(7-(2-amino-7-fluorobenzo[d]thiazol-4-yl)-8-fluoro-6-(furan-3-yl)-2-(((S)-1-methylpyrrolidin-2-yl)methoxy)quinazolin-4-yl)piperazin-1-yl)ethan-1-one (15 mg, 43%). LCMS (ES+) m/z 635.1 (M+H). ¹HNMR (400 MHz, CD₃OD) δ 7.96 (s, 1H), 7.33 (s, 1H), 7.19 (s, 1H), 7.11-7.17 (m, 1H), 6.98 (t, J=8.8 Hz, 1H), 6.15 (s, 1H), 4.90-5.08 (m, 2H), 4.70-4.80 (m, 1H), 4.14-4.29 (m, 4H), 4.04 (s, 2H), 3.92 (s, 3H), 3.77 (s, 3H), 3.10 (s, 3H), 2.36-2.50 (m, 1H), 2.18-2.30 (m, 1H), 2.05-2.18 (m, 2H).

### Synthetic Example 16: Synthesis of 7-fluoro-4-(8-fluoro-2-(((S)-1-methylpyrrolidin-2-yl)methoxy)-4-(piperazin-1-yl)-6-(pyridin-3-yl)quinazolin-7-yl)benzo[d]thiazol-2-amine (Compound 155)

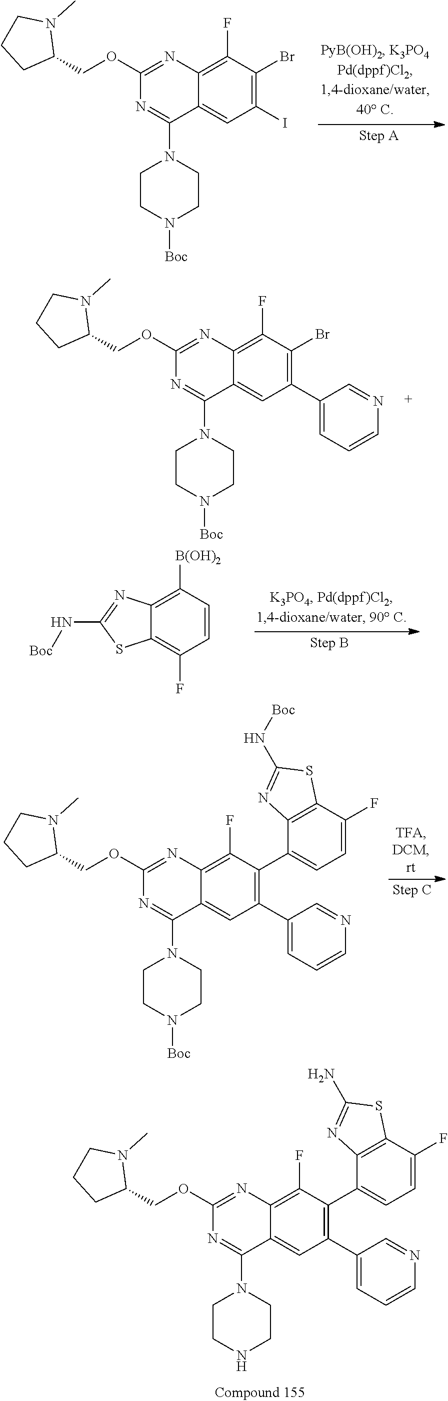

Step A: Preparation of tert-butyl (S)-4-(7-bromo-8-fluoro-2-((1-methylpyrrolidin-2-yl)methoxy)-6-(pyridin-3-yl)quinazolin-4-yl)piperazine-1-carboxylate: In a sealed tube was added tert-butyl-(S)-4-(7-bromo-8-fluoro-6-iodo-2-((1-methylpyrrolidin-2-yl)methoxy)quinazolin-4-yl)piperazine-1-carboxylate (40 mg, 0.062 mmol), 3-pyridylboronic acid (8.3 mg, 0.07 mmol) and potassium phosphate (39 mg, 0.18 mmol) in 1,4-dioxane (4 mL) and water (1.2 mL). The mixture was degassed with argon followed by the addition of Pd(dppf)Cl₂·DCM complex (4.5 mg, 0.006 mmol). The reaction mixture was degassed with argon and heated at 40° C. for 1 hour. After cooling to ambient temperature, the resulting mixture was concentrated under reduced pressure to give the crude residue which was purified by silica gel column chromatography using 10% MeOH in DCM as eluent to provide tert-butyl (S)-4-(7-bromo-8-fluoro-2-((1-methylpyrrolidin-2-yl)methoxy)-6-(pyridin-3-yl)quinazolin-4-yl)piperazine-1-carboxylate (17 mg, 46%) as a brown gum. LCMS ESI (+) m/z [M+H] 601.19. ¹HNMR (300 MHz, CDCl₃) δ 8.72-8.62 (m, 2H), 7.78 (ddd, J=5.4, 3.5, 1.8 Hz, 1H), 7.52 (d, J=1.6 Hz, 1H), 7.41 (dd, J=7.8, 5.0 Hz, 1H), 4.72 (dd, J=11.3, 5.2 Hz, 1H), 4.49 (dd, J=11.3, 5.9 Hz, 1H), 3.85-3.74 (m, 4H), 3.66-3.54 (m, 4H), 3.39-3.28 (m, 1H), 3.13-2.99 (m, 1H), 2.67 (s, 3H), 2.54-2.42 (m, 1H), 2.24-1.82 (m, 4H), 1.47 (s, 9H).

Step B: Preparation of tert-butyl 4-(7-(2-((tert-butoxycarbonyl)amino)-7-fluorobenzo[d]thiazol-4-yl)-8-fluoro-2-(((S)-1-methylpyrrolidin-2-yl)methoxy)-6-(pyridin-3-yl)quinazolin-4-yl)piperazine-1-carboxylate: To a mixture of tert-butyl (S)-4-(7-bromo-8-fluoro-2-((1-methylpyrrolidin-2-yl)methoxy)-6-(pyridin-3-yl)quinazolin-4-yl)piperazine-1-carboxylate (73 mg, 0.12 mmol) and [2-(tert-butoxycarbonylamino)-7-fluoro-1,3-benzothiazol-4-yl]boronic acid (42 mg, 0.13 mmol) in 1,4-dioxane (4 mL) and water (1.2 mL). The mixture was degassed with argon followed by the addition of Pd(dppf)Cl₂·DCM complex (8.9 mg, 0.012 mmol). The mixture was degassed with argon and heated at 90° C. for 2 hours. After cooling to ambient temperature, the resulting mixture was concentrated under reduced pressure to give the crude residue which was purified by silica gel column chromatography using 10% MeOH in DCM as eluent to give tert-butyl 4-(7-(2-((tert-butoxycarbonyl)amino)-7-fluorobenzo[d]thiazol-4-yl)-8-fluoro-2-(((S)-1-methylpyrrolidin-2-yl)methoxy)-6-(pyridin-3-yl)quinazolin-4-yl)piperazine-1-carboxylate (35 mg, 36%) as a brown gum. LCMS ESI (+) m/z 789.40 [M+H]. ¹HNMR (300 MHz, CDCl₃) δ 8.44 (s, 1H), 8.30 (s, 1H), 7.53 (s, 1H), 7.17-7.04 (m, 2H), 6.96-6.87 (m, 2H), 4.77-4.61 (m, 1H), 4.54-4.36 (m, 1H), 3.89-3.75 (m, 4H), 3.70-3.58 (m, 4H), 3.12-3.38 (m, 1H), 2.67 (s, 3H), 2.21-2.09 (m, 1H), 2.04-1.78 (m, 5H), 1.54 (s, 9H), 1.51 (s, 9H).

Step C: Preparation of 7-fluoro-4-(8-fluoro-2-(((S)-1-methylpyrrolidin-2-yl)methoxy)-4-(piperazin-1-yl)-6-(pyridin-3-yl)quinazolin-7-yl)benzo[d]thiazol-2-amine: To a solution of tert-butyl 4-(7-(2-((tert-butoxycarbonyl)amino)-7-fluorobenzo[d]thiazol-4-yl)-8-fluoro-2-(((S)-1-methylpyrrolidin-2-yl)methoxy)-6-(pyridin-3-yl)quinazolin-4-yl)piperazine-1-carboxylate (35 mg, 0.044 mmol) in DCM (1 mL) was added TFA (0.70 mL, 9.09 mmol) at 0° C. The mixture was stirred at room temperature for 2 hours. The reaction mixture was evaporated and co-evaporated with DCM to give 7-fluoro-4-(8-fluoro-2-(((S)-1-methylpyrrolidin-2-yl)methoxy)-4-(piperazin-1-yl)-6-(pyridin-3-yl)quinazolin-7-yl)benzo[d]thiazol-2-amine (25 mg, 96%) as a TFA salt. LCMS ESI (+) m/z 588.19 [M+H]. ¹HNMR (300 MHz, CD₃OD) δ 8.70 (br s, 2H), 8.15 (s, 1H), 7.99 (s, 1H), 7.84-7.61 (m, 4H), 7.27 (s, 1H), 6.99-6.89 (m, 1H), 4.99 (s, 1H), 4.75 (dd, J=12.5, 6.5 Hz, 1H), 4.21 (t, J=5.5 Hz, 4H), 3.94 (s, 1H), 3.78 (s, 1H), 3.57-3.45 (m, 4H), 3.13 (s, 3H), 2.50-2.38 (m, 1H), 2.29-2.07 (m, 3H).

### Synthetic Example 17: Synthesis of 4-(4-(3,8-diazabicyclo[3.2.1]octan-3-yl)-6-chloro-8-fluoro-2-((tetrahydro-1H-pyrrolizin-7a(5H)-yl)methoxy)quinazolin-7-yl)-7-fluorobenzo[d]thiazol-2-amine (Compound 157)

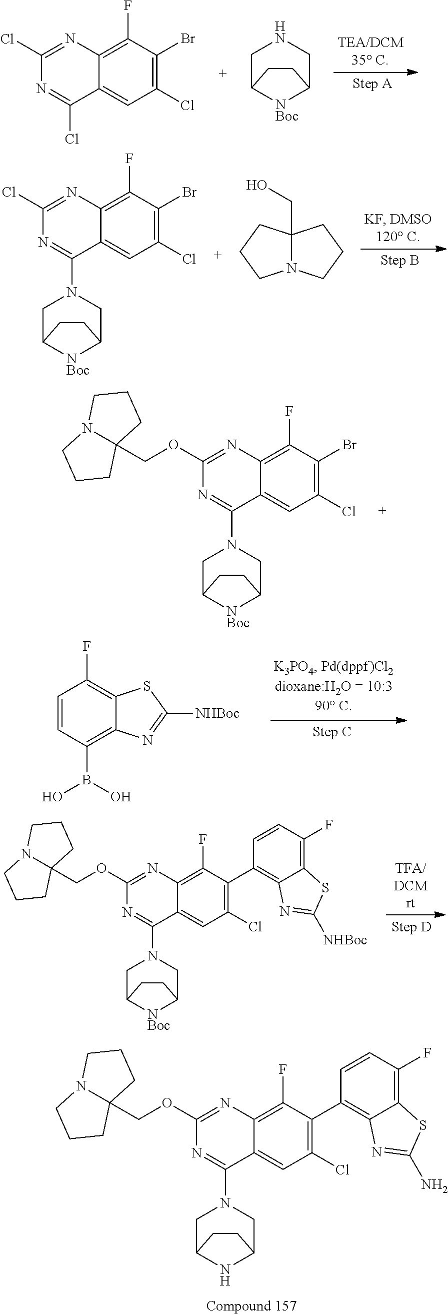

Step A: Preparation of tert-butyl-3-(7-bromo-2,6-dichloro-8-fluoro-quinazolin-4-yl)-3,8-diazabicyclo[3.2.1]octane-8-carboxylate: To a mixture of 7-bromo-2,4,6-trichloro-8-fluoroquinazoline (500 mg, 1.51 mmol) in DCM (10 mL) was added tert-butyl 3,8-diazabicyclo[3.2.1]octane-8-carboxylate (386 mg, 1.82 mmol) and Et₃N (459 mg, 4.54 mmol) at ambient temperature and stirred at ambient temperature for 2 hours. Ethyl acetate (50 mL) was added and washed with saturated brine solution. The organic layers were then separated, dried (MgSO₄), filtered, and concentrated under reduced pressure. The crude was then purified by flash chromatography on silica gel to give tert-butyl-3-(7-bromo-2,6-dichloro-8-fluoro-quinazolin-4-yl)-3,8-diazabicyclo[3.2.1]octane-8-carboxylate (608 mg, 79%). LCMS (ES+) m/z 505 (M+H).

Step B: Preparation of tert-butyl-3-[7-bromo-6-chloro-8-fluoro-2-(1,2,3,5,6,7-hexahydropyrrolizin-8-ylmethoxy)quinazolin-4-yl]-3,8-diazabicyclo[3.2.1]octane-8-carboxylate: To a mixture of tert-butyl-3-(7-bromo-2,6-dichloro-8-fluoro-quinazolin-4-yl)-3,8-diazabicyclo[3.2.1]octane-8-carboxylate (200 mg, 0.40 mmol) in DMSO (2 mL) was added 1,2,3,5,6,7-hexahydropyrrolizin-8-ylmethanol (167 mg, 1.19 mmol) and KF (69.1 mg, 1.19 mmol) at ambient temperature. The mixture was then stirred at 120° C. for 2 hours. After cooling to ambient temperature, ethyl acetate (20 mL) was added and washed with brine. The organic layers were then separated, dried (MgSO₄), and filtered before concentrating to dryness. The crude was then purified by flash column chromatography on silica gel eluting with 30% EtOAc in hexane. The desired fractions were concentrated to give tert-butyl-3-[7-bromo-6-chloro-8-fluoro-2-(1,2,3,5,6,7-hexahydropyrrolizin-8-ylmethoxy)quinazolin-4-yl]-3,8-diazabicyclo[3.2.1]octane-8-carboxylate (134 mg, 55%). LCMS (ES+) m/z 610 (M+H).

Step C: Preparation of tert-butyl-3-[7-[2-(tert-butoxycarbonylamino)-7-fluoro-1,3-benzothiazol-4-yl]-6-chloro-8-fluoro-2-(1,2,3,5,6,7-hexahydropyrrolizin-8-ylmethoxy)quinazolin-4-yl]-3,8-diazabicyclo[3.2.1]octane-8-carboxylate: To a mixture of tert-butyl-3-[7-bromo-6-chloro-8-fluoro-2-(1,2,3,5,6,7-hexahydropyrrolizin-8-ylmethoxy)quinazolin-4-yl]-3,8-diazabicyclo[3.2.1]octane-8-carboxylate (60 mg, 0.098 mmol) in water (1 mL) and 1,4-dioxane (2 mL) was added [2-(tert-butoxycarbonylamino)-7-fluoro-1,3-benzothiazol-4-yl]boronic acid (40 mg, 0.13 mmol), K₃PO₄ (62 mg, 0.30 mmol) and Pd(dtppf)Cl₂ (5.1 mg, 0.0079 mmol) at ambient temperature. The mixture was stirred at 90° C. for 1 hour under N₂. After cooling to ambient temperature, the reaction was concentrated to dryness and the residue was taken up in EtOAc (20 mL) and the organic layers washed with saturated brine solution. The organic layers were then separated, dried (MgSO₄), filtered, and concentrated under reduced pressure. The crude was then purified by flash chromatography on silica gel to give tert-butyl-3-[7-[2-(tert-butoxycarbonylamino)-7-fluoro-1,3-benzothiazol-4-yl]-6-chloro-8-fluoro-2-(1,2,3,5,6,7-hexahydropyrrolizin-8-ylmethoxy)quinazolin-4-yl]-3,8-diazabicyclo[3.2.1]octane-8-carboxylate (60 mg, 76%). LCMS (ES+) m/z 798.1 (M+H).

Step D: Preparation of 4-(4-(3,8-diazabicyclo[3.2.1]octan-3-yl)-6-chloro-8-fluoro-2-((tetrahydro-1H-pyrrolizin-7a(5H)-yl)methoxy)quinazolin-7-yl)-7-fluorobenzo[d]thiazol-2-amine: To a mixture of tert-butyl-3-[7-[2-(tert-butoxycarbonylamino)-7-fluoro-1,3-benzothiazol-4-yl]-6-chloro-8-fluoro-2-(1,2,3,5,6,7-hexahydropyrrolizin-8-ylmethoxy)quinazolin-4-yl]-3,8-diazabicyclo[3.2.1]octane-8-carboxylate (60 mg, 0.075 mmol) in DCM (2 mL) was added TFA (1.0 mL) at ambient temperature and stirred at this temperature for 1 hour. The reaction was concentrated under reduced pressure. The residue obtained was then purified by preparative RP-HPLC. The desired fractions were concentrated to give 4-(4-(3,8-diazabicyclo[3.2.1]octan-3-yl)-6-chloro-8-fluoro-2-((tetrahydro-1H-pyrrolizin-7a(5H)-yl)methoxy)quinazolin-7-yl)-7-fluorobenzo[d]thiazol-2-amine (25 mg, 55%). LCMS ESI (+) m/z 598.2 (M+H). ¹HNMR (400 MHz, CD₃OD) δ 7.95 (s, 1H), 7.22 (dd, J=5.6, 8.4 Hz, 1H), 7.02 (t, J=8.8 Hz, 1H), 4.68-4.75 (m, 2H), 4.65 (s, 2H), 4.18-4.32 (m, 2H), 3.84-3.92 (m, 2H), 3.63-3.71 (m, 2H), 3.24-3.29 (m, 2H), 2.28-2.36 (m, 2H), 2.19-2.26 (m, 2H), 2.0-2.18 (m, 8H).

### Synthetic Example 18: Synthesis of 4-(4-(3,8-diazabicyclo[3.2.1]octan-3-yl)-8-fluoro-2-((tetrahydro-1H-pyrrolizin-7a(5H)-yl)methoxy)-6-(trifluoromethyl)quinazolin-7-yl)-7-fluorobenzo[d]thiazol-2-amine (Compound 158)

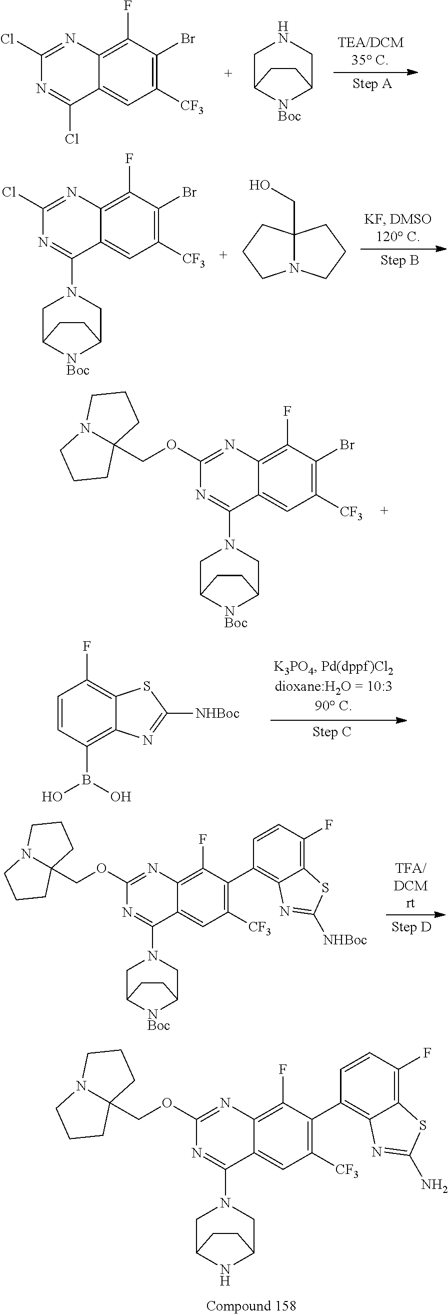

Step A: Preparation of tert-butyl-3-(7-bromo-2,6-dichloro-8-fluoro-quinazolin-4-yl)-3,8-diazabicyclo[3.2.1]octane-8-carboxylate: To a mixture of 7-bromo-2,4-dichloro-8-fluoro-6-(trifluoromethyl)quinazoline (200 mg, 0.55 mmol) in DCM (3 mL) was added tert-butyl 3,8-diazabicyclo[3.2.1]octane-8-carboxylate (128 mg, 0.60 mmol) and Et₃N (284 mg, 2.12 mmol) at ambient temperature and stirred at ambient temperature for 2 hours. Ethyl acetate (20 mL) was added and washed with saturated brine solution. The organic layers were then separated, dried (MgSO₄), filtered, and concentrated under reduced pressure. The crude was then purified directly on silica gel column, eluting with 30% EtOAc in petroleum ether to give tert-butyl-3-(7-bromo-2-chloro-8-fluoro-6-(trifluoromethyl)quinazolin-4-yl)-3,8-diazabicyclo[3.2.1]octane-8-carboxylate (144 mg, 48%). LCMS (ES+) m/z 530.0 (M+H).

Step B: Preparation of tert-butyl-3-(7-bromo-8-fluoro-2-((tetrahydro-1H-pyrrolizin-7a(5H)-yl)methoxy)-6-(trifluoromethyl)quinazolin-4-yl)-3,8-diazabicyclo[3.2.1]octane-8-carboxylate: To a mixture of tert-butyl (1R,5S)-3-(7-bromo-2-chloro-8-fluoro-6-(trifluoromethyl)quinazolin-4-yl)-3,8-diazabicyclo[3.2.1]octane-8-carboxylate (144 mg, 0.27 mmol) and KF (124 mg, 2.13 mmol) in DMSO (1 mL) was added (tetrahydro-1H-pyrrolizin-7a(5H)-yl)methanol (94 mg, 0.68 mmol) at ambient temperature. The mixture was then stirred at 120° C. for 2 hours. After cooling to ambient temperature, ethyl acetate (20 mL) was added and washed with brine. The organic layers were then separated, dried (MgSO₄), and filtered before concentrated to dryness. The crude was then purified by preparative-TLC (DCM/MeOH=10/1) to give tert-butyl (1R,5S)-3-(7-bromo-8-fluoro-2-((tetrahydro-1H-pyrrolizin-7a(5H)-yl)methoxy)-6-(trifluoromethyl)quinazolin-4-yl)-3,8-diazabicyclo[3.2.1]octane-8-carboxylate (47 mg, 27%). LCMS (ES+) m/z 644.1 (M+H).

Step C: Preparation of tert-butyl-3-(7-(2-((tert-butoxycarbonyl)amino)-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)-3,8-diazabicyclo[3.2.1]octane-8-carboxylate: To a mixture of tert-butyl-3-(7-bromo-8-fluoro-2-((tetrahydro-1H-pyrrolizin-7a(5H)-yl)methoxy)-6-(trifluoromethyl)quinazolin-4-yl)-3,8-diazabicyclo[3.2.1]octane-8-carboxylate (47 mg, 0.073 mmol) in water (1 mL) and 1,4-dioxane (2 mL) were added (2-((tert-butoxycarbonyl)amino)-7-fluorobenzo[d]thiazol-4-yl)boronic acid (25 mg, 0.080 mmol), Pd(dtbpf)Cl₂ (4.8 mg, 0.0073 mmol) and K₃PO₄ (31 mg, 0.15 mmol) at ambient temperature. The mixture was bubbled with argon for about 1-2 minutes and then sealed. After that, the mixture was stirred for 1 hour at 90° C. After cooling to ambient temperature, the reaction was concentrated to dryness and the residue was taken up in EtOAc (20 mL) and the organic layers washed with saturated brine solution. The organic layers were then separated, dried (MgSO₄), filtered, and concentrated under reduced pressure. The crude was then purified by preparative-TLC (DCM/MeOH=10/1) to give tert-butyl-3-(7-(2-((tert-butoxycarbonyl)amino)-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)-3,8-diazabicyclo[3.2.1]octane-8-carboxylate (31 mg, 50%). LCMS (ES+) m/z 832.3 (M+H).

Step D: Preparation of 4-(4-(3,8-diazabicyclo[3.2.1]octan-3-yl)-8-fluoro-2-((tetrahydro-1H-pyrrolizin-7a(5H)-yl)methoxy)-6-(trifluoromethyl)quinazolin-7-yl)-7-fluorobenzo[d]thiazol-2-amine: To a mixture of tert-butyl-3-(7-(2-((tert-butoxycarbonyl)amino)-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)-3,8-diazabicyclo[3.2.1]octane-8-carboxylate (31 mg, 0.037 mmol) in DCM (2 mL) was added TFA (2.8 mL, 36.1 mmol) at ambient temperature and stirred at this temperature for 2 hours. The reaction was concentrated under reduced pressure. The residue obtained was then purified by preparative RP-HPLC to give 4-(4-((1R,5S)-3,8-diazabicyclo[3.2.1]octan-3-yl)-8-fluoro-2-((tetrahydro-1H-pyrrolizin-7a(5H)-yl)methoxy)-6-(trifluoromethyl)quinazolin-7-yl)-7-fluorobenzo[d]thiazol-2-amine (8.1 mg, 34%). LCMS ESI (+) m/z 631.22 (M+H). ¹HNMR (400 MHz, CD₃OD) δ 8.17 (s, 1H), 7.25-7.15 (m, 1H), 7.00 (t, J=8.8 Hz, 1H), 4.77 (d, J=13.6 Hz, 2H), 4.67 (s, 2H), 4.20-4.30 (m, 2H), 3.95 (d, J=14.0 Hz, 2H), 3.61-3.78 (m, 2H), 3.24-3.32 (m, 2H), 2.46-1.97 (m, 12H).

### Synthetic Example 19: Synthesis of 4-(7-(2-amino-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)piperazine-1-carboxamide (Compound 165)

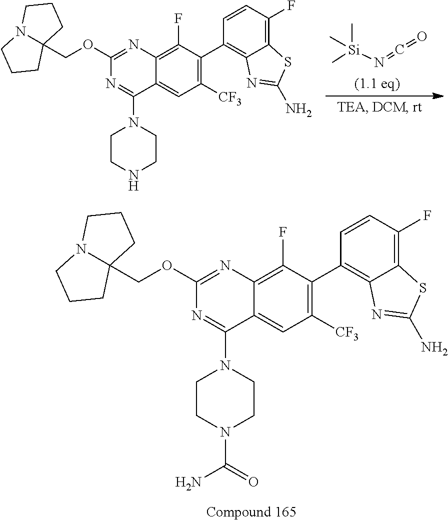

Preparation of 4-(7-(2-amino-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)piperazine-1-carboxamide: To a mixture of 7-fluoro-4-[8-fluoro-2-(1,2,3,5,6,7-hexahydropyrrolizin-8-ylmethoxy)-4-piperazin-1-yl-6-(trifluoromethyl)quinazolin-7-yl]-1,3-benzothiazol-2-amine (45 mg, 0.052 mmol) in DCM (3 mL) was added trimethylamine (16 mg, 0.16 mmol) and isocyanato(trimethyl)silane (6.0 mg, 0.052 mmol) at ambient temperature. The mixture was stirred for 2 hours at ambient temperature. The reaction was quenched with saturated aqueous sodium bicarbonate (20 mL) and extracted with EtOAc. The organic layers were washed with brine, separated, dried over Na₂SO₄, filtered, and evaporated to dryness. The crude product was purified by preparative RP-HPLC to give 4-[7-(2-amino-7-fluoro-1,3-benzothiazol-4-yl)-8-fluoro-2-(1,2,3,5,6,7-hexahydropyrrolizin-8-ylmethoxy)-6-(trifluoromethyl)quinazolin-4-yl]piperazine-1-carboxamide (14.8 mg, 42%). LCMS ESI (+) m/z 649.2 (M+H). ¹HNMR (400 MHz, CD₃OD) δ 8.26 (s, 1H), 7.22-7.25 (m, 1H), 7.02 (t, J=8.8 Hz, 1H), 4.68 (s, 2H), 4.11-4.18 (m, 4H), 3.65-3.74 (m, 6H), 3.24-3.28 (m, 2H), 2.06-2.36 (m, 8H).

### Synthetic Example 20: Synthesis of 2-[7-(2-amino-7-fluoro-1,3-benzothiazol-4-yl)-8-fluoro-2-[[(2S)-1-methylpyrrolidin-2-yl]methoxy]-4-piperazin-1-yl-quinazolin-6-yl]acetonitrile (Compound 175)

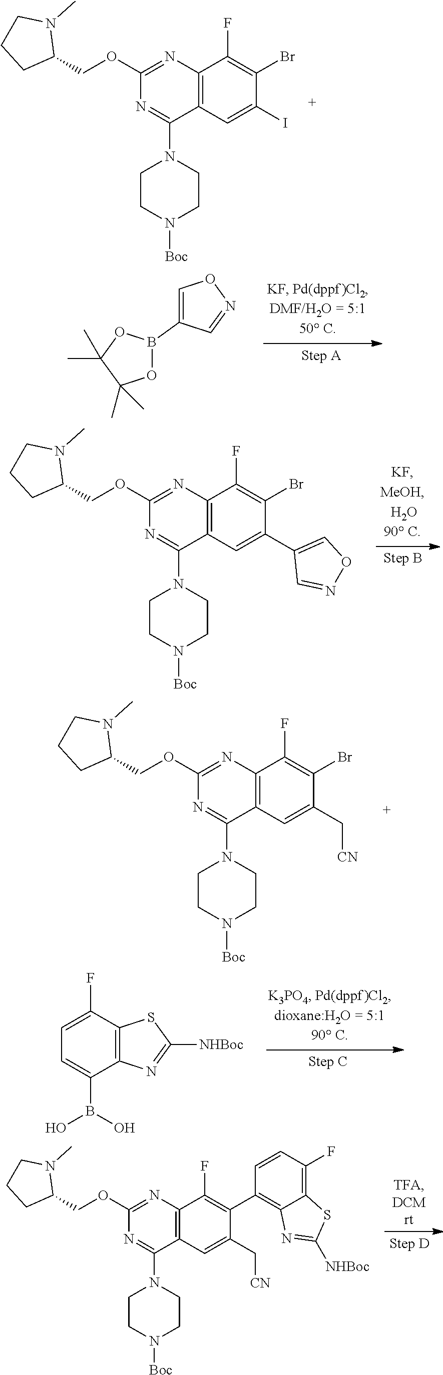

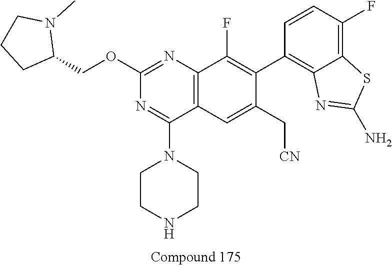 [SMILES: CN1CCC[C@H]1COc1nc(N2CCNCC2)c2cc(CC#N)c(-c3ccc(F)c4sc(N)nc34)c(F)c2n1]

Step A: Preparation of tert-butyl 4-[7-bromo-8-fluoro-6-isoxazol-4-yl-2-[[(2S)-1-methylpyrrolidin-2-yl]methoxy]quinazolin-4-yl]piperazine-1-carboxylate: To a solution of tert-butyl 4-[7-bromo-8-fluoro-6-iodo-2-[[(2S)-1-methylpyrrolidin-2-yl]methoxy]quinazolin-4-yl]piperazine-1-carboxylate (500 mg, 0.77 mmol) in DMF/water (5:1, 7.7 mL) were successively added 4-(4,4,5,5-tetramethyl-1,3,2-dioxaborolan-2-yl)isoxazole (180 mg, 0.92 mmol) and potassium fluoride (134 mg, 2.31 mmol) at ambient temperature. The mixture was purged with argon for 10 min and [1,1′-bis(diphenylphosphino)ferrocene] dichloropalladium(II) (56 mg, 0.077 mmol) was added. The mixture was purged another 5 min, then stirred at 50° C. for 3 hours. Upon completion, the mixture was cooled to ambient temperature and diluted with water (50 mL). The residue was taken up in ethyl acetate (3×50 mL), then the combined organic layers were washed with brine, dried over Na₂SO₄, filtered, and concentrated under reduced pressure. The residue was purified by normal phase chromatography on silica gel with 20% methanol in DCM to give tert-butyl 4-[7-bromo-8-fluoro-6-isoxazol-4-yl-2-[[(2S)-1-methylpyrrolidin-2-yl]methoxy]quinazolin-4-yl]piperazine-1-carboxylate (400 mg, 90%) as a light brown solid. LCMS ESI (+) m/z 593.06 (M+H). ¹HNMR (300 MHz, CDCl₃) δ 8.76 (s, 1H), 8.55 (s, 1H), 7.57 (s, 1H), 4.84-4.62 (m, 1H), 4.54-4.40 (m, 1H), 3.86-3.76 (m, 4H), 3.70-3.57 (m, 4H), 3.49-3.19 (m, 1H), 3.13-2.84 (m, 1H), 2.68 (s, 3H), 2.55-2.34 (m, 1H), 2.27-2.09 (m, 1H), 2.04-1.92 (m, 3H), 1.49 (s, 9H).

Step B: Preparation of tert-butyl 4-[7-bromo-6-(cyanomethyl)-8-fluoro-2-[(1-methylpyrrolidin-2-yl)methoxy]quinazolin-4-yl]piperazine-1-carboxylate: To a solution of tert-butyl 4-[7-bromo-8-fluoro-6-isoxazol-4-yl-2-[[(2S)-1-methylpyrrolidin-2-yl]methoxy]quinazolin-4-yl]piperazine-1-carboxylate (400 mg, 0.68 mmol) in methanol (3.4 mL) were successively added potassium fluoride (118 mg, 2.03 mmol) and water (0.34 mL) at ambient temperature. The mixture was stirred at 90° C. for 4 hours. Upon completion, the mixture was concentrated under reduced pressure and the residue was purified by normal phase chromatography on silica gel with 20% methanol in DCM to give tert-butyl 4-[7-bromo-6-(cyanomethyl)-8-fluoro-2-[(1-methylpyrrolidin-2-yl)methoxy]quinazolin-4-yl]piperazine-1-carboxylate (295 mg, 77%) as a light yellow solid. LCMS ESI (+) m/z 565.21 (M+H). ¹HNMR (300 MHz, CDCl₃) δ 7.82 (s, 1H), 4.64-4.50 (m, 1H), 4.42-4.29 (m, 1H), 3.95 (s, 2H), 3.80-3.77 (m, 4H), 3.68-3.58 (m, 4H), 3.24-3.07 (m, 1H), 2.88-2.71 (m, 1H), 2.56 (s, 3H), 2.44-2.27 (m, 1H), 2.21-2.02 (m, 1H), 1.93-1.69 (m, 3H), 1.49 (s, 9H).

Step C: Preparation of tert-butyl 4-[7-[2-(tert-butoxycarbonylamino)-7-fluoro-1,3-benzothiazol-4-yl]-6-(cyanomethyl)-8-fluoro-2-[[(2S)-1-methylpyrrolidin-2-yl]methoxy]quinazolin-4-yl]piperazine-1-carboxylate: To a solution of tert-butyl 4-[7-bromo-6-(cyanomethyl)-8-fluoro-2-[(1-methylpyrrolidin-2-yl)methoxy]quinazolin-4-yl]piperazine-1-carboxylate (40 mg, 0.07 mmol) in 1, 4-dioxane/water (5:1, 1.1 mL) were successively added potassium phosphate (45 mg, 0.21 mmol) and [2-(tert-butoxycarbonylamino)-7-fluoro-1,3-benzothiazol-4-yl]boronic acid (33 mg, 0.11 mmol) at ambient temperature. The mixture was purged with argon for 10 minutes and [1,1′-bis(diphenylphosphino)ferrocene] dichloropalladium(II) (8 mg, 0.011 mmol) was added. The mixture was purged another 5 min then stirred at 90° C. for 1 hour. Upon completion, the mixture was concentrated under reduced pressure and the residue was purified by normal phase chromatography on silica gel with 20% methanol in DCM to give tert-butyl 4-[7-[2-(tert-butoxycarbonylamino)-7-fluoro-1,3-benzothiazol-4-yl]-6-(cyanomethyl)-8-fluoro-2-[[(2S)-1-methylpyrrolidin-2-yl]methoxy]quinazolin-4-yl]piperazine-1-carboxylate (40 mg, 75%) as a light brown solid. LCMS ESI (+) m/z 751.05 (M+H). ¹H NMR (300 MHz, CD₃OD) δ 7.94 (s, 1H), 7.41 (dd, J=8.4, 5.3 Hz, 1H), 7.18 (t, J=8.8 Hz, 1H), 4.51 (d, J=5.4 Hz, 2H), 3.97-3.88 (m, 4H), 3.82-3.65 (m, 6H), 3.26-3.12 (m, 1H), 3.05-2.96 (m, 1H), 2.63 (s, 3H), 2.53 (q, J=9.0 Hz, 1H), 2.19-2.10 (m, 1H), 1.97-1.79 (m, 3H), 1.54 (s, 9H), 1.51 (s, 9H).

Step D: Preparation of 2-[7-(2-amino-7-fluoro-1,3-benzothiazol-4-yl)-8-fluoro-2-[[(2S)-1-methylpyrrolidin-2-yl]methoxy]-4-piperazin-1-yl-quinazolin-6-yl]acetonitrile: To a solution of tert-butyl 4-[7-[2-(tert-butoxycarbonylamino)-7-fluoro-1,3-benzothiazol-4-yl]-6-(cyanomethyl)-8-fluoro-2-[[(2S)-1-methylpyrrolidin-2-yl]methoxy]quinazolin-4-yl]piperazine-1-carboxylate (40 mg, 0.05 mmol) in DCM (1 mL) was added trifluoroacetic acid (0.25 mL) and the mixture was stirred at ambient temperature for 2 hours. Upon completion, the residue was concentrated under reduced pressure to give 2-[7-(2-amino-7-fluoro-1,3-benzothiazol-4-yl)-8-fluoro-2-[[(2S)-1-methylpyrrolidin-2-yl]methoxy]-4-piperazin-1-yl-quinazolin-6-yl]acetonitrile (47 mg, quantitative yield) as a trifluoroacetic acid salt. LCMS ESI (+) m/z 551.23 (M+H). ¹HNMR (300 MHz, CD₃OD) δ 7.98 (s, 1H), 7.27 (dd, J=8.5, 5.4 Hz, 1H), 7.04 (t, J=8.8 Hz, 1H), 4.69 (dd, J=13.0, 6.4 Hz, 1H), 4.15 (t, J=4.8 Hz, 4H), 3.94-3.73 (m, 4H), 3.53 (t, J=5.4 Hz, 4H), 3.10 (s, 3H), 2.45-2.37 (m, 1H), 2.27-2.06 (m, 4H).

### Synthetic Example 21: Synthesis of 4-(6-ethyl-8-fluoro-2-(((S)-1-methylpyrrolidin-2-yl)methoxy)-4-(piperazin-1-yl)quinazolin-7-yl)-7-fluorobenzo[d]thiazol-2-amine (Compound 177)

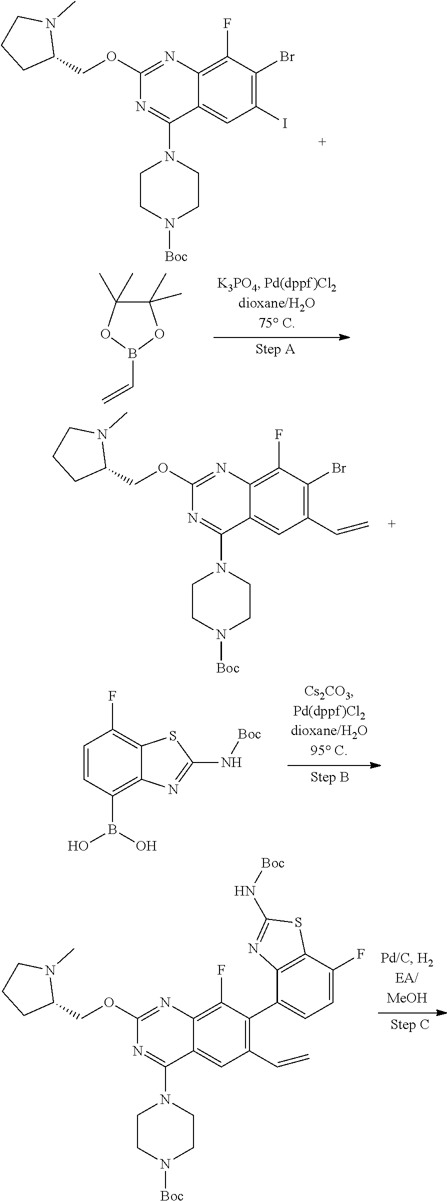

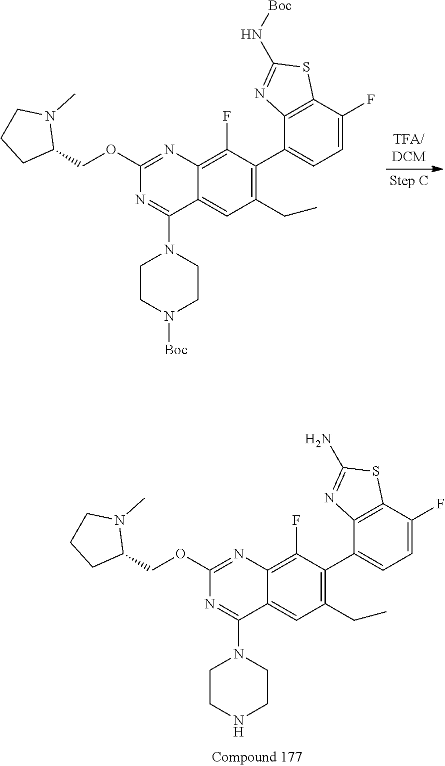 [SMILES: CCc1cc2c(N3CCN(C(=O)OC(C)(C)C)CC3)nc(OC[C@@H]3CCCN3C)nc2c(F)c1-c1ccc(F)c2sc(NC(=O)OC(C)(C)C)nc12 ; CCc1cc2c(N3CCNCC3)nc(OC[C@@H]3CCCN3C)nc2c(F)c1-c1ccc(F)c2sc(N)nc12]

Step A: Preparation of tert-butyl (S)-4-(7-bromo-8-fluoro-2-((1-methylpyrrolidin-2-yl)methoxy)-6-vinylquinazolin-4-yl)piperazine-1-carboxylate: In a sealed tube was added tert-butyl (S)-4-(7-bromo-8-fluoro-6-iodo-2-((1-methylpyrrolidin-2-yl)methoxy)quinazolin-4-yl)piperazine-1-carboxylate (0.25 g, 0.38 mmol), 4,4,5,5-tetramethyl-2-vinyl-1,3,2-dioxaborolane (0.065 g, 0.42 mmol), and potassium carbonate (0.11 g, 0.77 mmol) followed by water (0.16 mL) and 1,4-dioxane (1.6 mL). The mixture was degassed with argon followed by the addition of Pd(dppf)Cl₂·DCM complex (0.028 g, 0.038 mmol). The mixture was degassed with argon and heated at 75° C. for 3 hrs. After cooling to ambient temperature, the reaction mixture was added ethyl acetate and brine. The organic layer was collected, dried over sodium sulfate, filtered, and evaporated. The mixture was purified on a 40 g column with DCM to 10% MeOH in DCM to provide tert-butyl (S)-4-(7-bromo-8-fluoro-2-((1-methylpyrrolidin-2-yl)methoxy)-6-vinylquinazolin-4-yl)piperazine-1-carboxylate (0.15 g, 701%). LCMS ESI (+) m/z 572.22 (M+Na). ¹HNMR (300 MHz, CDCl₃) δ 7.76 (s, 1H), 7.12 (dd, J=17.2, 11.0 Hz, 1H), 5.70 (d, J=17.3 Hz, 1H), 5.44 (d, J=11.0 Hz, 1H), 4.58 (m, 1H), 4.37 (m, 1H), 3.82 (m, 4H), 3.67 (m, 4H), 3.16 (m, 1H), 3.03-2.66 (m, 1H), 2.49 (s, 3H), 2.38 (m, 1H), 2.10 (m, 1H), 1.83 (m, 3H), 1.44 (s, 9H).

Step B: Preparation of tert-butyl 4-(7-(2-((tert-butoxycarbonyl)amino)-7-fluorobenzo[d]thiazol-4-yl)-8-fluoro-2-(((S)-1-methylpyrrolidin-2-yl)methoxy)-6-vinylquinazolin-4-yl)piperazine-1-carboxylate: To a mixture of (S)-4-(7-bromo-8-fluoro-2-((1-methylpyrrolidin-2-yl)methoxy)-6-vinylquinazolin-4-yl)piperazine-1-carboxylate, cesium carbonate (0.15 g, 0.27 mmol) and [2-(tert-butoxycarbonylamino)-7-fluoro-1,3-benzothiazol-4-yl]boronic acid (0.17 g, 0.55 mmol) in 1,4-dioxane (1.3 mL) and water (0.13 mL) was degassed, followed by the addition of Pd(dppf)Cl₂·DCM complex (0.030 g, 0.041 mmol). The reaction mixture was degassed and heated at 90° C. for 1.5 hr. After cooling to ambient temperature, the reaction mixture was added to ethyl acetate and brine. The organic layer was collected, dried over sodium sulfate, filtered, and evaporated. The mixture was purified on a 40 g column with DCM to 10% MeOH in DCM to provide tert-butyl 4-(7-(2-((tert-butoxycarbonyl)amino)-7-fluorobenzo[d]thiazol-4-yl)-8-fluoro-2-(((S)-1-methylpyrrolidin-2-yl)methoxy)-6-vinylquinazolin-4-yl)piperazine-1-carboxylate (0.14 g, 69%). LCMS ESI (+) m/z 738.54 (M+H). ¹HNMR (300 MHz, CDCl₃) δ 7.88 (s, 1H), 7.13 (d, J=9.0 Hz, 1H), 6.39 (dd, J=17.6, 11.1 Hz, 1H), 5.61 (d, J=17.2 Hz, 1H), 5.13 (m, 1H), 4.70-4.50 (m, 1H), 4.47-4.08 (m, 4H), 3.84 (m, 4H), 3.68 (m, 1H), 3.33-3.00 (m, 1H), 2.77 (m, 1H), 2.55 (s, 3H), 2.43-2.24 (m, 1H), 2.41-2.19 (m, 1H), 1.81 (m, 3H), 1.54 (2s, 18H).

Step C: Preparation of tert-butyl 4-(7-(2-((tert-butoxycarbonyl)amino)-7-fluorobenzo[d]thiazol-4-yl)-6-ethyl-8-fluoro-2-(((S)-1-methylpyrrolidin-2-yl)methoxy)quinazolin-4-yl)piperazine-1-carboxylate: tert-butyl 4-(7-(2-((tert-butoxycarbonyl)amino)-7-fluorobenzo[d]thiazol-4-yl)-8-fluoro-2-(((S)-1-methylpyrrolidin-2-yl)methoxy)-6-vinylquinazolin-4-yl)piperazine-1-carboxylate (0.14 g, 0.20 mmol) in ethyl acetate (20 mL) and MeOH (10 mL) was added 10% palladium on carbon (0.021 mg). The reaction was placed under 1 atmosphere of hydrogen and stirred at ambient temperature for 1 hour. The reaction mixture was filtered over Celite® and washed with DCM. The mixture was evaporated and purified on a 12 g column with DCM to 10% MeOH in DCM to provide tert-butyl 4-(7-(2-((tert-butoxycarbonyl)amino)-7-fluorobenzo[d]thiazol-4-yl)-6-ethyl-8-fluoro-2-(((S)-1-methylpyrrolidin-2-yl)methoxy)quinazolin-4-yl)piperazine-1-carboxylate (0.10 g, 69%). LCMS ESI (+) m/z 740.41 (M+H). ¹H NMR (300 MHz, CDCl₃) δ 7.52 (s, 1H), 7.24 (m, 1H), 7.11 (t, J=8.7 Hz, 1H), 4.92-4.58 (m, 1H), 4.58-4.22 (m, 1H), 3.81 (m, 4H), 3.68 (m, 4H), 2.65 (s, 3H), 2.48 (m, 3H), 2.06 (m, 1H), 1.86 (m, 5H), 1.54 (2s, 18H), 1.05 (t, J=7.5 Hz, 3H).

Step D: Preparation of 4-(6-ethyl-8-fluoro-2-(((S)-1-methylpyrrolidin-2-yl)methoxy)-4-(piperazin-1-yl)quinazolin-7-yl)-7-fluorobenzo[d]thiazol-2-amine: To a solution of tert-butyl 4-(7-(2-((tert-butoxycarbonyl)amino)-7-fluorobenzo[d]thiazol-4-yl)-6-ethyl-8-fluoro-2-(((S)-1-methylpyrrolidin-2-yl)methoxy)quinazolin-4-yl)piperazine-1-carboxylate (0.10 g, 0.14 mmol) in DCM (10 mL) was added TFA (0.11 mL, 1.38 mmol). After completion the reaction mixture was evaporated and co-evaporated with DCM to provide 4-(6-ethyl-8-fluoro-2-(((S)-1-methylpyrrolidin-2-yl)methoxy)-4-(piperazin-1-yl)quinazolin-7-yl)-7-fluorobenzo[d]thiazol-2-amine as TFA salt (0.125 g). LCMS ESI (+) m/z 540.28 (M+H) and 562.26 (M+23). ¹H NMR (300 MHz, CD₃OD) δ 7.79 (s, 1H), 7.27 (m, 1H), 7.13 (m, 1H), 4.94 (m, 1H), 4.77 (m, 1H), 4.27 (m, 4H), 3.93 (m, 1H), 3.77 (m, 1H), 3.54 (m, 4H), 3.30-3.17 (m, 1H), 3.11 (s, 3H), 2.61 (dt, J=14.2, 7.5 Hz, 2H), 2.42 (m, 1H), 2.08 (m, 3H), 1.09 (t, J=7.4 Hz, 3H).

### Synthetic Example 22: Synthesis of 4-(4-(3,6-diazabicyclo[3.1.1]heptan-3-yl)-6-chloro-8-fluoro-2-((tetrahydro-1H-pyrrolizin-7a(5H)-yl)methoxy)quinazolin-7-yl)-7-fluorobenzo[d]thiazol-2-amine (Compound 184)

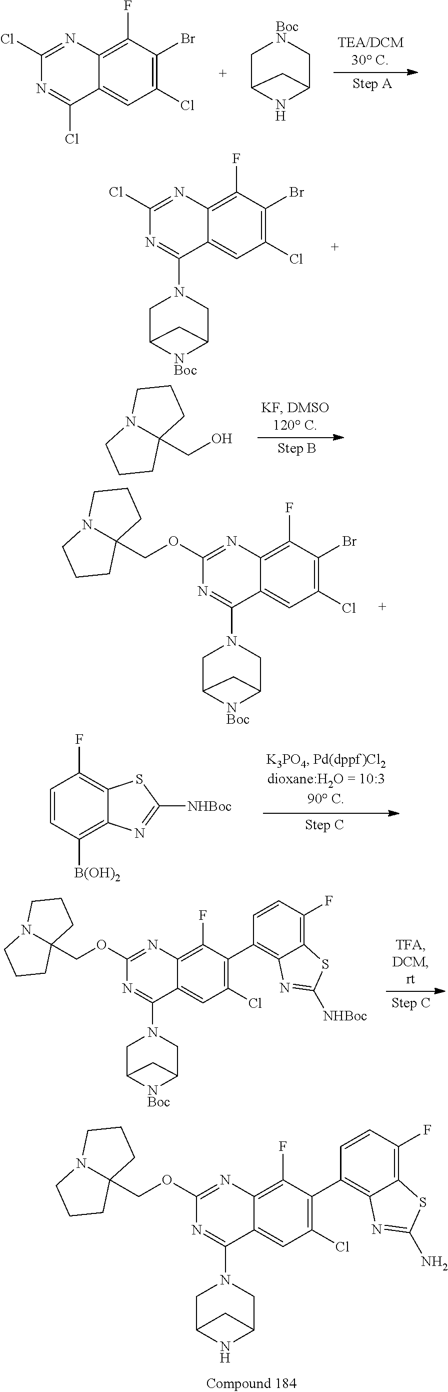

Step A: Preparation of tert-butyl 3-(7-bromo-2,6-dichloro-8-fluoro-quinazolin-4-yl)-3,6-diazabicyclo [3.1.1]heptane-6-carboxylate: To a solution of 7-bromo-2,4,6-trichloro-8-fluoroquinazoline (300 mg, 0.91 mmol) and tert-butyl 3,6-diazabicyclo[3.1.1]heptane-3-carboxylate (216 mg, 1.09 mmol) in DCM (5 mL) was added triethylamine (0.38 mL, 2.72 mmol) at 0° C. After addition, the resulting mixture was stirred at ambient temperature for 3 hours. The mixture was concentrated in vacuo and purified by preparative-TLC (EtOAc/PE=1/5) to give methyl 2-amino-4-bromo-3-fluorobenzoate (5.0 g, 94%) as a solid. LCMS ESI (+) m/z 490.9 (M+H).

Step B: Preparation of tert-butyl-3-[7-bromo-6-chloro-8-fluoro-2-(1,2,3,5,6,7-hexahydropyrrolizin-8-ylmethoxy) quinazolin-4-yl]-3,6-diazabicyclo[3.1.1]heptane-6-carboxylate: A suspension of tert-butyl 3-(7-bromo-2,6-dichloro-8-fluoro-quinazolin-4-yl)-3,6-diazabicyclo[3.1.1]heptane-6-carboxylate (200 mg, 0.41 mmol), 1,2,3,5,6,7-hexahydropyrrolizin-8-ylmethanol (172 mg, 1.22 mmol) and potassium fluoride (189 mg, 3.25 mmol) in DMSO (2 mL) was stirred at 90° C. for 4 hours. The mixture was diluted with EtOAc and washed with brine. The organic phase was dried over anhydrous Na2SO4, filtered, and concentrated in vacuo. The residue was purified by preparative-TLC (DCM/MeOH=20/1) to give tert-butyl-3-[7-bromo-6-chloro-8-fluoro-2-(1,2,3,5,6,7-hexahydropyrrolizin-8-ylmethoxy) quinazolin-4-yl]-3,6-diazabicyclo[3.1.1]heptane-6-carboxylate (100 mg, 41%) as a yellow solid. LCMS ESI (+) m/z 598.0 (M+H).

Step C: Preparation of tert-butyl 3-[7-[2-(tert-butoxycarbonylamino)-7-fluoro-1,3-benzothiazol-4-yl]-6-chloro-8-fluoro-2-(1,2,3,5,6,7-hexahydropyrrolizin-8-ylmethoxy)quinazolin-4-yl]-3,6-diazabicyclo[3.1.1]heptane-6-carboxylate: A suspension of tert-butyl 3-[7-bromo-6-chloro-8-fluoro-2-(1,2,3,5,6,7-hexahydropyrrolizin-8-ylmethoxy)quinazolin-4-yl]-3,6-diazabicyclo[3.1.1]heptane-6-carboxylate (150 mg, 0.25 mmol), [2-(tert-butoxycarbonylamino)-7-fluoro-1,3-benzothiazol-4-yl]boronic acid (1.30 eq, 102 mg, 0.327 mmol), potassium phosphate (160 mg, 0.75 mmol) and Pd(dtbpf)Cl₂ (16 mg, 0.025 mmol) in 1,4-dioxane (5 mL) and water (1 mL) was stirred at 90° C. for 2 hours. After cooling to ambient temperature, the mixture was diluted with EtOAc. The organic phase was washed with brine. The organic phase was dried over anhydrous Na₂SO₄, filtered, and concentrated in vacuo. The residue was purified by preparative-TLC (DCM/MeOH=15/1) to give tert-butyl 3-[7-[2-(tert-butoxycarbonylamino)-7-fluoro-1,3-benzothiazol-4-yl]-6-chloro-8-fluoro-2-(1,2,3,5,6,7-hexahydropyrrolizin-8-ylmethoxy)quinazolin-4-yl]-3,6-diazabicyclo[3.1.1]heptane-6-carboxylate (80 mg, 40%) as solid. LCMS ESI (+) m/z 783.28 (M+H).

Step D: Preparation of 4-(4-(3,6-diazabicyclo[3.1.1]heptan-3-yl)-6-chloro-8-fluoro-2-((tetrahydro-1H-pyrrolizin-7a(5H)-yl)methoxy)quinazolin-7-yl)-7-fluorobenzo[d]thiazol-2-amine: To a solution of tert-butyl 3-[7-[2-(tert-butoxycarbonylamino)-7-fluoro-1,3-benzothiazol-4-yl]-6-chloro-8-fluoro-2-(1,2,3,5,6,7-hexahydropyrrolizin-8-ylmethoxy)quinazolin-4-yl]-3,6-diazabicyclo[3.1.1]heptane-6-carboxylate (40 mg, 0.051 mmol) in DCM (4 mL) was added trifluoroacetic acid (1.0 mL, 13.0 mmol) at 0° C. After addition, the resulting mixture was stirred at ambient temperature for 1 hour. The mixture was concentrated in vacuo and purified by preparative RP-HPLC to give 4-(4-(3,6-diazabicyclo[3.1.1]heptan-3-yl)-6-chloro-8-fluoro-2-((tetrahydro-1H-pyrrolizin-7a(5H)-yl)methoxy)quinazolin-7-yl)-7-fluorobenzo[d]thiazol-2-amine (11 mg, 35%) as solid. LCMS ESI (+) m/z 584.0 (M+H). ¹HNMR (400 MHz, CD₃OD) δ 8.39 (d, J=1.2 Hz, 1H), 7.26 (dd, J=8.6, 5.6 Hz, 1H), 7.04 (t, J=9.2 Hz, 1H), 4.83 (s, 1H), 4.79 (s, 1H), 4.68 (d, J=2.8 Hz, 2H), 4.57-4.64 (m, 4H), 3.66-3.72 (m, 2H), 3.26-3.29 (m, 2H), 3.09-3.15 (m, 1H), 2.07-2.34 (m, 8H), 2.02 (d, J=10.8 Hz, 1H).

### Synthetic Example 23: Synthesis of 4-(6-cyclopropyl-8-fluoro-2-(((S)-1-methylpyrrolidin-2-yl)methoxy)-4-(piperazin-1-yl)quinazolin-7-yl)-7-fluorobenzo[d]thiazol-2-amine (Compound 200)

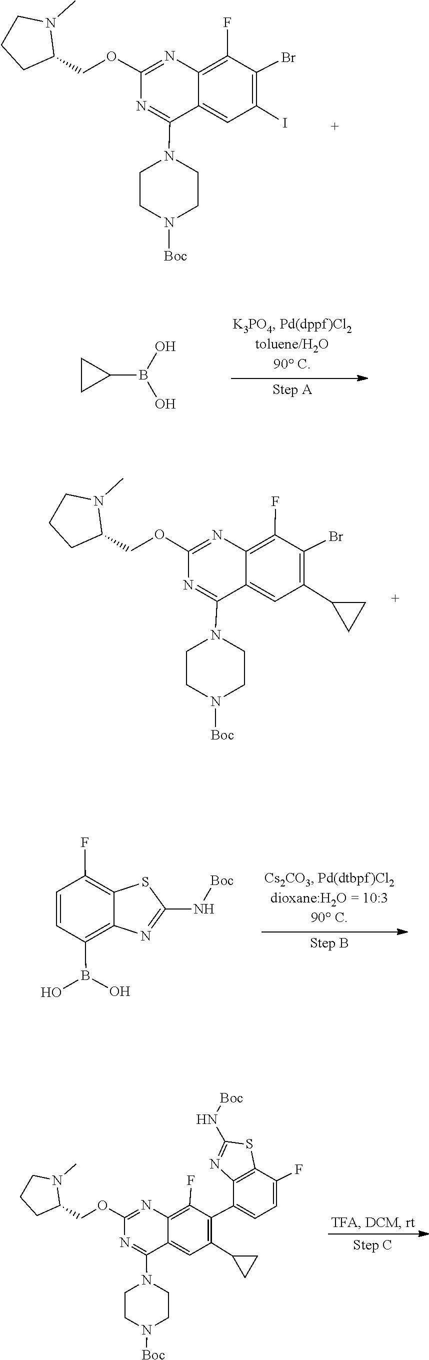 [SMILES: CN1CCC[C@H]1COc1nc(N2CCN(C(=O)OC(C)(C)C)CC2)c2cc(I)c(Br)c(F)c2n1 ; OB(O)C1CC1 ; CN1CCC[C@H]1COc1nc(N2CCN(C(=O)OC(C)(C)C)CC2)c2cc(C3CC3)c(Br)c(F)c2n1 ; CC(C)(C)OC(=O)Nc1nc2c(B(O)O)ccc(F)c2s1 ; CN1CCC[C@H]1COc1nc(N2CCN(C(=O)OC(C)(C)C)CC2)c2cc(C3CC3)c(-c3ccc(F)c4sc(NC(=O)OC(C)(C)C)nc34)c(F)c2n1]

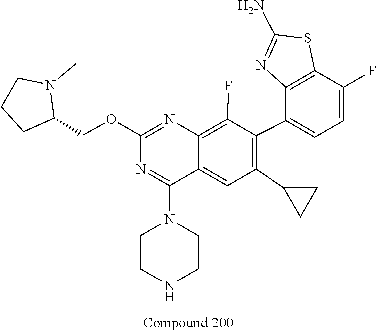 [SMILES: CN1CCC[C@H]1COc1nc(N2CCNCC2)c2cc(C3CC3)c(-c3ccc(F)c4sc(N)nc34)c(F)c2n1]

Step A: Preparation of tert-butyl (S)-4-(7-bromo-6-cyclopropyl-8-fluoro-2-((1-methylpyrrolidin-2-yl)methoxy)quinazolin-4-yl)piperazine-1-carboxylate. In a sealed tube were added tert-butyl 4-[7-bromo-8-fluoro-6-iodo-2-[[(2S)-1-methylpyrrolidin-2-yl]methoxy]quinazolin-4-yl]piperazine-1-carboxylate (0.100 g, 0.154 mmol), cyclopropylboronic acid (0.026 g, 0.308 mmol), Pd(dppf)Cl₂DCM complex (0.011 g, 0.015 mmol) followed by toluene (1.5 mL) and water (0.150 mL). The mixture was degassed with argon followed by the addition of K₃PO₄ (0.065 g, 0.308 mmol). The mixture was heated at 90° C. for 3 hours. To the reaction mixture was added ethyl acetate and water, and then the organic layer was collected, dried over sodium sulfate, filtered, and evaporated. The mixture was purified over a 25 g column with DCM to 15% MeOH in DCM to provide of tert-butyl (S)-4-(7-bromo-6-cyclopropyl-8-fluoro-2-((1-methylpyrrolidin-2-yl)methoxy)quinazolin-4-yl)piperazine-1-carboxylate (0.050 g, 0.089 mmol, 57%). LCMS ESI (+) m/z 586.13 (M+Na). ¹HNMR (300 MHz, CDCl₃) δ 7.23 (s, 1H), 4.59 (dd, J=10.9, 4.7 Hz, 1H), 4.35 (dd, J=10.7, 6.6 Hz, 1H), 3.71 (m, 4H), 3.70-3.55 (m, 4H), 3.17 (m, 1H), 2.81 (m, 1H), 2.56 (s, 3H), 2.38 (m, 1H), 2.17 (m, 1H), 2.09 (m, 1H), 1.85 (m, 3H), 1.52 (s, 9H), 1.21-1.01 (m, 2H), 0.82-0.47 (m, 2H).

Step B: Preparation of tert-butyl 4-(7-(2-((tert-butoxycarbonyl)amino)-7-fluorobenzo[d]thiazol-4-yl)-6-cyclopropyl-8-fluoro-2-(((S)-1-methylpyrrolidin-2-yl)methoxy)quinazolin-4-yl)piperazine-1-carboxylate. To a mixture of tert-butyl (S)-4-(7-bromo-6-cyclopropyl-8-fluoro-2-((1-methylpyrrolidin-2-yl)methoxy)quinazolin-4-yl)piperazine-1-carboxylate (0.05 g, 0.088 mmol), cesium carbonate (0.058 g, 0.177 mmol) and [2-(tert-butoxycarbonylamino)-7-fluoro-1,3-benzothiazol-4-yl]boronic acid (0.055 g, 0.177 mmol) in 1,4-dioxane (1.3 mL) and water (0.130 mL) was degassed followed by the addition of Pd(dppf)Cl₂ DCM complex (0.0097 g, 0.0133 mmol). The reaction mixture was degassed and heated at 90° C. for 1.5 hr. The mixture was extracted with EA-NaCl. The EA was collected, dried over sodium sulfate, filtered, and evaporated. The mixture was purified on a 12 g column with DCM to 15% MeOH in DCM. The material was purified again on 24 g column with 5% MeOH in DCM to 10% MeOH in DCM to give of tert-butyl 4-(7-(2-((tert-butoxycarbonyl)amino)-7-fluorobenzo[d]thiazol-4-yl)-6-cyclopropyl-8-fluoro-2-(((S)-1-methylpyrrolidin-2-yl)methoxy)quinazolin-4-yl)piperazine-1-carboxylate (0.010 g, 15%) enough pure for the next step. LCMS ESI (+) m/z 751.88 (M+H). ¹HNMR (300 MHz, Acetone-d₆) δ 7.54-7.43 (m, 1H), 7.43 (s, 1H), 7.25 (t, J=8.8 Hz, 1H), 4.47 (m, 1H), 4.30 (m, 1H), 3.82 (m, 4H), 3.69 (m, 4H), 3.11 (m, 1H), 2.82 (m, 2H), 2.50 (s, 3H), 2.32 (m, 1H), 1.89-1.65 (m, 4H), 1.56 (s, 9H), 1.49 (s, 9H), 0.86-0.45 (m, 4H).

Step C: Preparation of 4-(6-cyclopropyl-8-fluoro-2-(((S)-1-methylpyrrolidin-2-yl)methoxy)-4-(piperazin-1-yl)quinazolin-7-yl)-7-fluorobenzo[d]thiazol-2-amine. To a solution of tert-butyl 4-(7-(2-((tert-butoxycarbonyl)amino)-7-fluorobenzo[d]thiazol-4-yl)-6-cyclopropyl-8-fluoro-2-(((S)-1-methylpyrrolidin-2-yl)methoxy)quinazolin-4-yl)piperazine-1-carboxylate (0.010 g, 0.0133 mmol) in DCM (10 mL) was added an excess of TFA. After completion the reaction mixture was evaporated to provide 4-(6-cyclopropyl-8-fluoro-2-(((S)-1-methylpyrrolidin-2-yl)methoxy)-4-(piperazin-1-yl)quinazolin-7-yl)-7-fluorobenzo[d]thiazol-2-amine (0.012 g) as TFA salts. LCMS ESI (+) m/z 574.13 (M+H). ¹H NMR (300 MHz, CD₃OD) δ 7.44 (s, 1H), 7.32 (m, 1H), 7.13 (m, 1H), 4.95 (m, 1H), 4.73 (m, 1H), 4.33-4.10 (m, 4H), 3.92 (m, 1H), 3.75 (m, 1H), 3.51 (m, 4H), 3.11 (s, 3H), 2.44 (m, 1H), 2.06 (m, 4H), 1.74 (m, 1H), 1.02-0.49 (m, 4H).

### Synthetic Example 24: Synthesis of 1-[7-(2-amino-7-fluoro-1,3-benzothiazol-4-yl)-8-fluoro-2-[[(2S)-1-methylpyrrolidin-2-yl]methoxy]-4-piperazin-1-yl-quinazolin-6-yl]cyclobutanecarbonitrile (Compound 203)

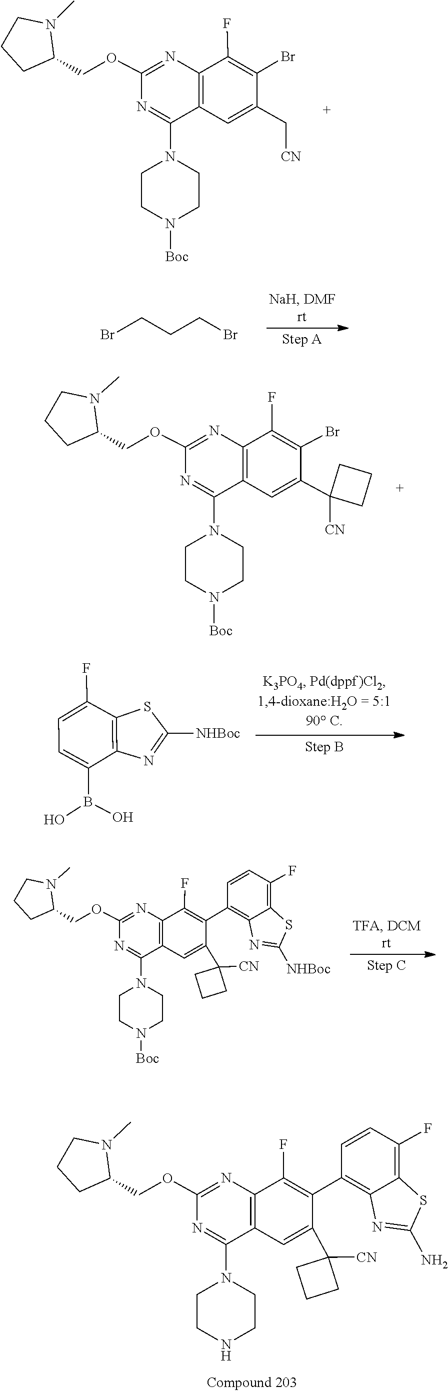

Step A: Preparation of tert-butyl 4-[7-bromo-6-(1-cyanocyclobutyl)-8-fluoro-2-[[(2S)-1-methylpyrrolidin-2-yl]methoxy]quinazolin-4-yl]piperazine-1-carboxylate: To a solution of tert-butyl 4-[7-bromo-6-(cyanomethyl)-8-fluoro-2-[(1-methylpyrrolidin-2-yl)methoxy]quinazolin-4-yl]piperazine-1-carboxylate (296 mg, 0.53 mmol) in DMF (4.8 mL) were successively added sodium hydride (48 mg, 1.2 mmol, 60% dispersion in mineral oil) and 1,3-dibromopropane (0.96 mL, 0.48 mmol, 0.5M in DMF) dropwise at 0° C. After addition, the mixture was warmed to ambient temperature and stirred at ambient temperature for 16 hours. Upon completion, the mixture was cooled to 0° C. and diluted with water (20 mL). The residue was taken up in ethyl acetate (3×20 mL), then the combined organic layers were washed with brine, dried over Na₂SO₄, filtered, and concentrated under reduced pressure. The residue was purified by normal phase chromatography on silica gel with 20% methanol in DCM to give tert-butyl 4-[7-bromo-6-(1-cyanocyclobutyl)-8-fluoro-2-[[(2S)-1-methylpyrrolidin-2-yl]methoxy]quinazolin-4-yl]piperazine-1-carboxylate (93 mg, 32%) as a light tan solid. LCMS ESI (+) m/z 625.18 (M+Na). ¹HNMR (300 MHz, CDCl₃) δ 7.43 (d, J=1.7 Hz, 1H), 4.54 (dd, J=10.8, 4.7 Hz, 1H), 4.33 (dd, J=10.8, 6.7 Hz, 1H), 3.76 (dd, J=6.8, 3.4 Hz, 4H), 3.63 (dd, J=6.8, 3.5 Hz, 4H), 3.19-2.97 (m, 3H), 2.76-2.56 (m, 4H), 2.50 (s, 3H), 2.28 (q, J=9.0 Hz, 1H), 2.12-1.97 (m, 2H), 1.90-1.65 (m, 3H), 1.49 (s, 9H).

Step B: Preparation of tert-butyl 4-[7-[2-(tert-butoxycarbonylamino)-7-fluoro-1,3-benzothiazol-4-yl]-6-(1-cyanocyclobutyl)-8-fluoro-2-[[(2S)-1-methylpyrrolidin-2-yl]methoxy]quinazolin-4-yl]piperazine-1-carboxylate: To a solution of tert-butyl 4-[7-bromo-6-(1-cyanocyclobutyl)-8-fluoro-2-[[(2S)-1-methylpyrrolidin-2-yl]methoxy]quinazolin-4-yl]piperazine-1-carboxylate (50 mg, 0.08 mmol) in 1,4-dioxane/water (5:1, 1.1 mL) were successively added potassium phosphate (53 mg, 0.25 mmol) and [2-(tert-butoxycarbonylamino)-7-fluoro-1,3-benzothiazol-4-yl]boronic acid (39 mg, 0.12 mmol) at ambient temperature. The mixture was purged with argon for 10 minutes and [1,1′-bis(diphenylphosphino) ferrocene]dichloropalladium(II) (9 mg, 0.012 mmol) was added. The mixture was purged another 5 minutes then stirred at 90° C. for 1 hour. Upon completion, the mixture was concentrated under reduced pressure and the residue was purified by normal phase chromatography on silica gel with 20% methanol in DCM to give tert-butyl 4-[7-[2-(tert-butoxycarbonylamino)-7-fluoro-1,3-benzothiazol-4-yl]-6-(1-cyanocyclobutyl)-8-fluoro-2-[[(2S)-1-methylpyrrolidin-2-yl]methoxy]quinazolin-4-yl]piperazine-1-carboxylate (13 mg, 20%) as a brown solid. LCMS ESI (+) m/z 791.05 (M+H). ¹H NMR (300 MHz, CDCl₃) δ 7.48 (q, J=4.7, 4.3 Hz, 2H), 7.14 (t, J=8.6 Hz, 1H), 4.65 (t, J=6.9 Hz, 1H), 4.43 (dd, J=11.2, 6.1 Hz, 1H), 3.85-3.73 (m, 4H), 3.70-3.58 (m, 4H), 3.38-3.13 (m, 1H), 2.79-2.67 (m, 1H), 2.61-2.46 (m, 6H), 2.44-2.17 (m, 3H), 2.15-1.98 (m, 2H), 1.94-1.69 (m, 4H), 1.53 (s, 9H), 1.51 (s, 9H).

Step C: Preparation of 1-[7-(2-amino-7-fluoro-1,3-benzothiazol-4-yl)-8-fluoro-2-[[(2S)-1-methylpyrrolidin-2-yl]methoxy]-4-piperazin-1-yl-quinazolin-6-yl]cyclobutanecarbonitrile: To a solution of tert-butyl 4-[7-[2-(tert-butoxycarbonylamino)-7-fluoro-1,3-benzothiazol-4-yl]-6-(1-cyanocyclobutyl)-8-fluoro-2-[[(2S)-1-methylpyrrolidin-2-yl]methoxy]quinazolin-4-yl]piperazine-1-carboxylate (13 mg, 0.02 mmol) in DCM (1 mL) was added trifluoroacetic acid (0.25 mL) and the mixture was stirred at ambient temperature for 2 hours. Upon completion, the residue was concentrated under reduced pressure to give 1-[7-(2-amino-7-fluoro-1,3-benzothiazol-4-yl)-8-fluoro-2-[[(2S)-1-methylpyrrolidin-2-yl]methoxy]-4-piperazin-1-yl-quinazolin-6-yl]cyclobutanecarbonitrile (18 mg, quantitative yield) as a trifluoroacetic acid salt. LCMS ESI (+) m/z 590.79 (M+H). ¹H NMR (300 MHz, CD₃OD) δ 7.67 (s, 1H), 7.32 (dd, J=8.4, 5.5 Hz, 1H), 7.03 (t, J=8.8 Hz, 1H), 4.77-4.53 (m, 1H), 4.15 (d, J=5.4 Hz, 5H), 3.97-3.85 (m, 1H), 3.84-3.73 (m, 1H), 3.50 (d, J=5.4 Hz, 5H), 3.10 (d, J=6.6 Hz, 3H), 2.89-2.66 (m, 1H), 2.45-2.34 (m, 1H), 2.26-2.09 (m, 5H), 1.86-1.76 (m, 1H), 1.68-1.57 (m, 1H). One proton was merged with water residual peak.

### Synthetic Example 25: Synthesis of 7-fluoro-4-(8-fluoro-6-methoxy-4-(piperazin-1-yl)-2-((tetrahydro-1H-pyrrolizin-7a(5H)-yl)methoxy)quinazolin-7-yl)benzo[d]thiazol-2-amine (Compound 216)

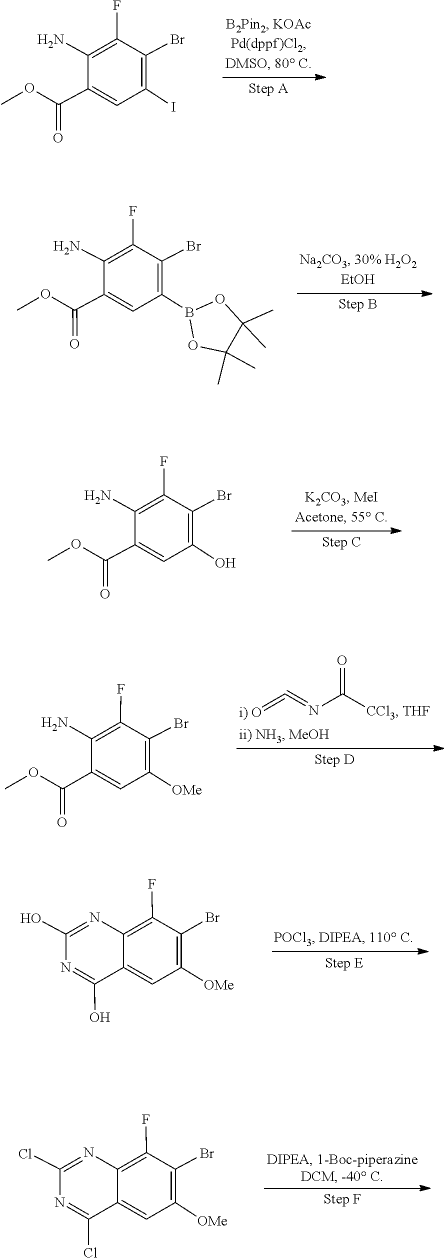

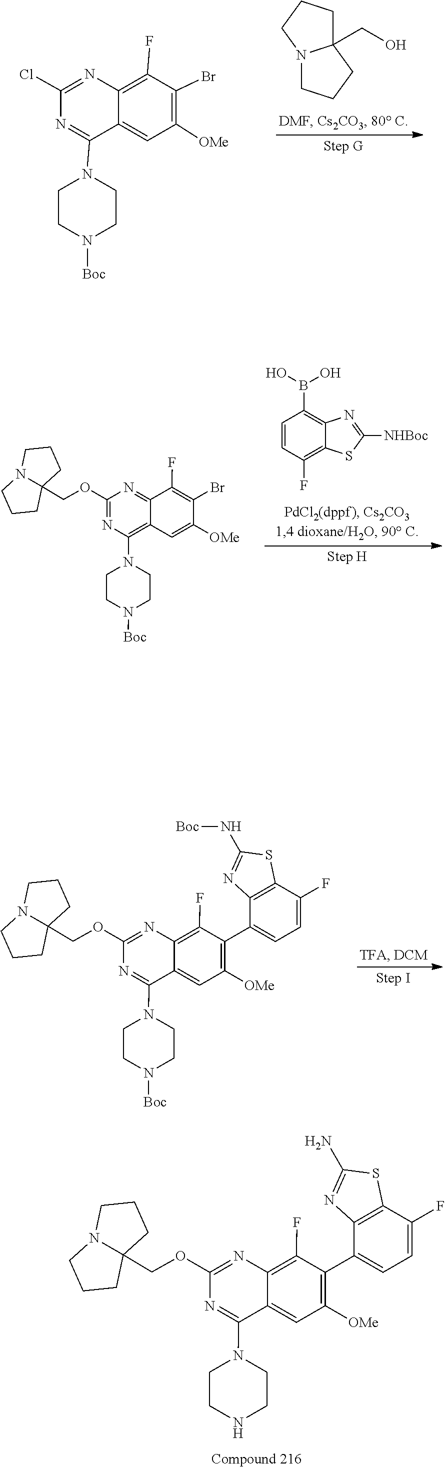 [SMILES: COc1cc2c(N3CCN(C(=O)OC(C)(C)C)CC3)nc(Cl)nc2c(F)c1Br ; OCC12CCCN1CCC2 ; COc1cc2c(N3CCN(C(=O)OC(C)(C)C)CC3)nc(OCC34CCCN3CCC4)nc2c(F)c1Br ; CC(C)(C)OC(=O)Nc1nc2c(B(O)O)ccc(F)c2s1 ; COc1cc2c(N3CCN(C(=O)OC(C)(C)C)CC3)nc(OCC34CCCN3CCC4)nc2c(F)c1-c1ccc(F)c2sc(NC(=O)OC(C)(C)C)nc12 ; COc1cc2c(N3CCNCC3)nc(OCC34CCCN3CCC4)nc2c(F)c1-c1ccc(F)c2sc(N)nc12]

Step A: Preparation of methyl 2-amino-4-bromo-3-fluoro-5-(4,4,5,5-tetramethyl-1,3,2-dioxaborolan-2-yl)benzoate: In a sealed tube, methyl 2-amino-4-bromo-3-fluoro-5-iodobenzoate (5.43 g, 14.5 mmol) was dissolved in anhydrous DMSO (54 mL). To this stirring solution, bis(pinacolato)diboron (3.87 g, 15.2 mmol) and KOAc (4.28 g, 43.6 mmol) were added and degassed with argon for 10 minutes, then added Pd(dppf)Cl₂·DCM (0.59 g, 0.73 mmol) and degassed for another 10 minutes, capped the sealed tube. The reaction mixture was directly dipped into a preheated oil bath at 70° C. and heated at 80° C. for 14 hours. The reaction mixture was cooled to room temperature and poured into ice water. The aqueous layer was extracted with EtOAc (3×100 mL). The organic layers were combined and washed with water (2×20 mL), dried over Na₂SO₄, and concentrated in vacuo. The residue was purified by flash chromatography on silica gel (eluted with a gradient of 5-7% EtOAc in hexane) to provide title compound (2.9 g, 54%) as light-yellow solid. MS (ESI+) m/z 374.08 [M+H]. ¹HNMR (300 MHz, CDCl₃) δ 8.00 (d, J=1.7 Hz, 1H), 6.09 (br s, 2H), 3.89 (s, 3H), 1.35 (s, 12H).

Step B: Preparation of methyl 2-amino-4-bromo-3-fluoro-5-hydroxybenzoate: To a stirring solution of methyl 2-amino-4-bromo-3-fluoro-5-(4,4,5,5-tetramethyl-1,3,2-dioxaborolan-2-yl)benzoate (2.90 g, 7.75 mmol) in ethanol (80 mL), K₂CO₃ (1.37 g, 12.9 mmol) and hydrogen peroxide (6.30 mL, 61.5 mmol) were added subsequently at 0° C. and gradually warmed the reaction mixture to room temperature and continued the stirring for 20 hours. The reaction mixture was concentrated to half in vacuo and added water and EtOAc. Layers were separated and the aqueous layer was further extracted with EtOAc (2×70 mL). The organic layers were combined, dried over Na₂SO₄, filtered, and concentrated. The crude was purified by flash column chromatography on silica gel (eluted with a gradient of 20-25% EtOAc in hexane) provided desired material (1.24 g, 61%) as off-white solid. MS(ESI+) m/z 263.93 [M+H]. ¹HNMR (300 MHz, CDCl₃) δ 7.36 (d, J=2.2 Hz, 1H), 5.47 (s, 2H), 4.96 (s, 1H), 3.88 (s, 3H).

Step C: Preparation of methyl 2-amino-4-bromo-3-fluoro-5-methoxybenzoate: To a stirring solution of methyl 2-amino-4-bromo-3-fluoro-5-hydroxy-benzoate (0.58 g, 2.20 mmol) in acetone (8 mL), K₂CO₃ (0.16 g, 6.59 mmol) and iodomethane (165 μL, 2.64 mmol) were added at room temperature under argon. The reaction mixture was heated to 55° C. After 24 hours of heating at 55° C., the reaction mixture was filtered to remove K₂CO₃ and concentrated. The resulted crude was purified by column chromatography on silica gel with gradient of EtOAc in hexane to provide methyl 2-amino-4-bromo-3-fluoro-5-methoxybenzoate (0.40 g, 66%) as white solid. ¹HNMR (300 MHz, CDCl₃) δ 7.18 (d, J=2.1 Hz, 1H), 5.55 (s, 2H), 3.90 (s, 3H), 3.85 (s, 3H).

Step D: Preparation of methyl 4-bromo-3-fluoro-5-methoxy-2-(3-(2,2,2-trichloroacetyl)ureido)benzoate. To a stirring solution of methyl 2-amino-4-bromo-3-fluoro-5-methoxy-benzoate (0.21 g, 0.75 mmol) in THE (2 mL), 2,2,2-trichloroacetyl isocyanate (0.13 mL, 1.12 mmol) was added at room temperature under argon. After 1 hour, the reaction mixture was concentrated in vacuo and used it for next step without any purification. ¹HNMR (300 MHz, CDCl₃) δ 10.24 (s, 1H), 8.60 (s, 1H), 7.32 (d, J=1.9 Hz, 1H), 3.98 (s, 3H), 3.95 (s, 3H).

Preparation of 7-bromo-8-fluoro-6-methoxyquinazoline-2,4-diol: To a stirring suspension of above methyl 4-bromo-3-fluoro-5-methoxy-2-(3-(2,2,2-trichloroacetyl)ureido)benzoate (0.35 g, 0.75 mmol) in methanol, 7 N ammonia (0.25 mL, 1.73 mmol) in methanol was added at room temperature and vigorously stirred at room temperature. Within 15 minutes after addition of 7 N ammonia in methanol, white solid was precipitating out. The reaction was continued for 17 hours then concentrated in vacuo and co-distilled with methanol twice provided crude of desired material (300 mg, cal. 100%) as a white solid which was used for next step without any purification. LCMS (ESI+) m/z 288.71 [M+H].

Step E: Preparation of 7-bromo-2,4-dichloro-8-fluoro-6-methoxyquinazoline: POCl₃ (0.97 mL, 10.4 mmol) was charged to a round bottom flask containing 7-bromo-8-fluoro-6-methoxy-quinazoline-2,4-diol (0.30 g, 1.04 mmol) under nitrogen and cooled to 0° C. To this, DIPEA (0.36 mL, 2.08 mmol) was added dropwise and stirred for 10 minutes. Then the reaction temperature was raised to 110° C. and stirred at this temperature for 2 hours. After cooling to ambient temperature, the reaction mixture was concentrated in vacuo and co-distilled with DCM multiple times. This residue was redissolved in DCM, washed with water, and saturated NH₄C₁ solution subsequently, dried over Na₂SO₄ and concentrated in vacuo. The crude was purified by flash column chromatography on silica gel (eluted with 5% EtOAc in hexane to 100% EtOAc) provided desired material (200 mg, 59%). LCMS (ESI+) m/z 324.66 [M+H]. ¹HNMR (300 MHz, CDCl₃) δ 7.27 (d, J=1.9 Hz, 1H), 4.11 (s, 3H).

Step F: Preparation of tert-butyl 4-(7-bromo-2-chloro-8-fluoro-6-methoxyquinazolin-4-yl)piperazine-1-carboxylate. A solution of 7-bromo-2,4-dichloro-8-fluoro-6-methoxy-quinazoline (0.20 g, 0.61 mmol) in DCM (1.2 mL) was stirred at −40° C. under argon. To this, DIPEA (0.32 mL, 1.84 mmol) and 1-Boc-piperazine (0.14 mg, 0.74 mmol) were added subsequently, and the reaction mixture was allowed to warm to room temperature and stirred for 40 minutes. The reaction mixture was concentrated, and the resultant crude was purified by flash chromatography on silica gel with a gradient of DCM/methanol to provide title compound (0.18 g, 62%) as a white solid. LCMS(ESI+) m/z 497.05 [M+Na]. ¹HNMR (300 MHz, CDCl₃) δ 6.89 (d, J=1.9 Hz, 1H), 3.99 (s, 3H), 3.87-3.60 (m, 8H), 1.50 (s, 9H).

Step G: Preparation of tert-butyl 4-(7-bromo-8-fluoro-6-methoxy-2-((tetrahydro-1H-pyrrolizin-7a(5H)-yl)methoxy)quinazolin-4-yl)piperazine-1-carboxylate: A stirring suspension of tert-butyl 4-(7-bromo-2-chloro-8-fluoro-6-methoxy-quinazolin-4-yl)piperazine-1-carboxylate (0.15 g, 0.32 mmol) in 1,4-dioxane (6 mL) was cooled to 0° C. To this, 1,2,3,5,6,7-hexahydropyrrolizin-8-ylmethanol (0.22 mL, 1.58 mmol) and Cs₂CO₃ (205 mg, 0.631 mmol) were added, and the reaction mixture was heated to 120° C. and stirred for 48 hours. After cooling to ambient temperature, the reaction mixture was concentrated and obtained residue was purified by flash chromatography on silica gel (eluted with a gradient of 60-80% DCM/Methanol) to provide desired material (36 mg, 20%). LCMS(ESI+) m/z 581.82 [M+H] and 582.76 [M+2+H]. ¹H NMR (300 MHz, CDCl₃) δ 6.89 (s, 1H), 4.75 (s, 2H), 4.06-3.53 (m, 13H, OMe peak at 3.95 ppm is in this cluster of peaks), 3.09-2.86 (m, 2H), 2.42 (dt, J=13.0, 7.1 Hz, 2H), 2.36-1.90 (m, 6H), 1.49 (m, 9H).

Step H: Preparation of tert-butyl 4-(7-(2-((tert-butoxycarbonyl)amino)-7-fluorobenzo[d]thiazol-4-yl)-8-fluoro-6-methoxy-2-((tetrahydro-1H-pyrrolizin-7a(5H)-yl)methoxy)quinazolin-4-yl)piperazine-1-carboxylate: To a stirring suspension of tert-butyl 4-(7-bromo-8-fluoro-6-methoxy-2-((tetrahydro-1H-pyrrolizin-7a(5H)-yl)methoxy)quinazolin-4-yl)piperazine-1-carboxylate (40 mg, 0.069 mmol) in 1,4-dioxane (1.5 mL), (2-((tert-butoxycarbonyl)amino)-7-fluorobenzo[d]thiazol-4-yl)boronic acid (43.0 mg, 0.14 mmol) and an aqueous solution of Cs₂CO₃ (45 mg, 0.14 mmol) in water (0.15 mL). The reaction mixture was degassed with argon for 10 minutes then Pd(dppf)Cl₂ (8.44 mg, 0.010 mmol) was added and degassed again for 10 minutes. The reaction mixture was heated to 90° C. for 90 minutes. The reaction mixture was cooled to room temperature and diluted with EtOAc and water. Layers were separated and the aqueous layer was further extracted with EtOAc (2×15 mL). The organic layers were combined, washed with brine, dried over Na₂SO₄, filtered, and concentrated in vacuo. The resultant crude was purified by flash chromatography on silica gel (eluted with 40-50% EtOAc in hexane) to provide the desired material (17 mg, 32%). LCMS(ESI+) m/z 767.93 [M+H]. ¹HNMR (300 MHz, CDCl₃) δ 7.35 (dd, J=8.3, 5.3 Hz, 1H), 7.08 (t, J=8.6 Hz, 1H), 6.98 (s, 1H), 4.74 (s, 2H), 3.98-3.60 (m, 13H), 3.12-2.88 (m, 2H), 2.52-2.36 (m, 2H), 2.31-1.90 (m, 6H), 1.53 (s, 9H), 1.50 (s, 9H).

Step I: Preparation of 7-fluoro-4-(8-fluoro-6-methoxy-4-(piperazin-1-yl)-2-((tetrahydro-1H-pyrrolizin-7a(5H)-yl)methoxy)quinazolin-7-yl)benzo[d]thiazol-2-amine: To a stirring solution tert-butyl 4-(7-(2-amino-7-fluorobenzo[d]thiazol-4-yl)-8-fluoro-6-methoxy-2-((tetrahydro-1H-pyrrolizin-7a(5H)-yl)methoxy)quinazolin-4-yl)piperazine-1-carboxylate (17 mg, 0.022 mmol) in DCM (3 mL) at 0° C., TFA (0.15 mL) was added and the reaction was allowed to stir at room temperature. After 2 hours of stirring at room temperature, the reaction mixture was concentrated in vacuo and co-distilled with DCM multiple times and dried under high vacuum afforded desired material (22 mg, brown liquid, quant.) as TFA salt. MS(ESI+) m/z 568.25 [M+H]. ¹HNMR (300 MHz, CD₃OD) δ 7.28 (dd, J=8.4, 5.4 Hz, 1H), 7.18 (s, 1H), 7.06 (t, J=8.8 Hz, 1H), 4.67 (s, 2H), 4.23-4.07 (m, 4H), 3.88 (s, 3H), 3.69 (dt, J=11.7, 5.7 Hz, 2H), 3.51 (t, J=6.7 Hz, 4H), 3.30-3.23 (m, 2H; portions of these peaks were merged with CD₃OD solvent peak), 2.41-2.05 (m, 8H).

### Synthetic Example 26: Synthesis of 4-(6-(difluoromethyl)-8-fluoro-2-(((S)-1-methylpyrrolidin-2-yl)methoxy)-4-(piperazin-1-yl)quinazolin-7-yl)-7-fluorobenzo[d]thiazol-2-amine (Compound 217)

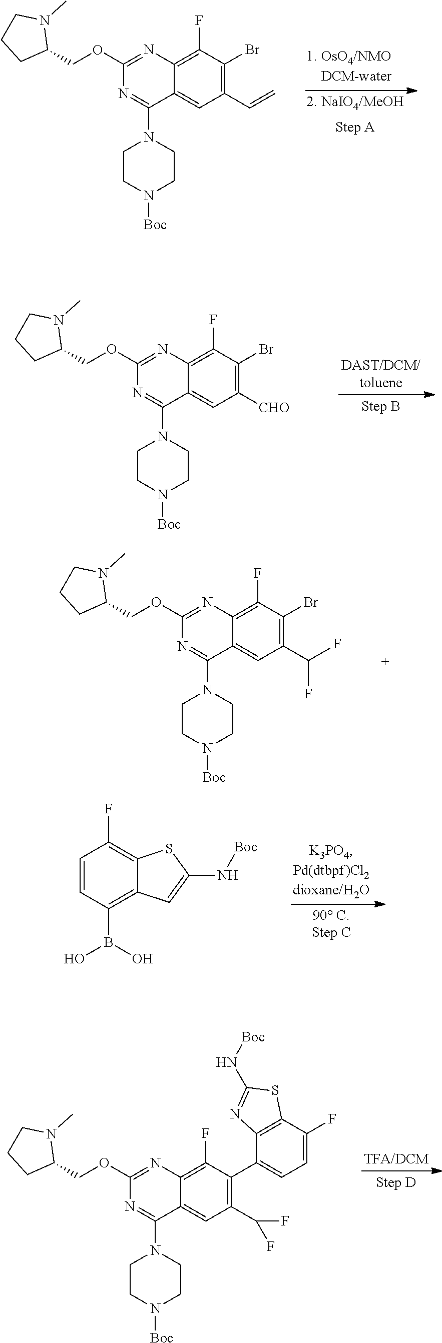 [SMILES: C=Cc1cc2c(N3CCN(C(=O)OC(C)(C)C)CC3)nc(OC[C@@H]3CCCN3C)nc2c(F)c1Br ; CN1CCC[C@H]1COc1nc(N2CCN(C(=O)OC(C)(C)C)CC2)c2cc(C=O)c(Br)c(F)c2n1 ; CN1CCC[C@H]1COc1nc(N2CCN(C(=O)OC(C)(C)C)CC2)c2cc(C(F)F)c(Br)c(F)c2n1 ; CC(C)(C)OC(=O)Nc1cc2c(B(O)O)ccc(F)c2s1 ; CN1CCC[C@H]1COc1nc(N2CCN(C(=O)OC(C)(C)C)CC2)c2cc(C(F)F)c(-c3ccc(F)c4sc(NC(=O)OC(C)(C)C)nc34)c(F)c2n1]

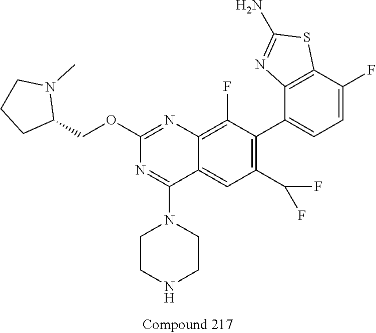 [SMILES: CN1CCC[C@H]1COc1nc(N2CCNCC2)c2cc(C(F)F)c(-c3ccc(F)c4sc(N)nc34)c(F)c2n1]

Step A: Preparation of tert-butyl (S)-4-(7-bromo-8-fluoro-6-formyl-2-((1-methylpyrrolidin-2-yl)methoxy)quinazolin-4-yl)piperazine-1-carboxylate: To a mixture of tert-butyl (S)-4-(7-bromo-8-fluoro-2-((1-methylpyrrolidin-2-yl)methoxy)-6-vinylquinazolin-4-yl)piperazine-1-carboxylate (0.20 g, 0.36 mmol) in DCM (1.1 mL) and water (0.29 mL) were added a catalytic amount of osmium tetraoxide and NMO (0.085 g, 0.723 mmol) at room temperature. The mixture was stirred at ambient temperature overnight. DCM and water were added. Organic layer was separated, dried (sodium sulfate), filtered, and concentrated under reduced pressure. The residue obtained was purified on a 12 g silica gel column and eluted with DCM to 30% MeOH in DCM to provide 70 mg diol intermediate. To the diol intermediate (0.070 g, 0.011 mmol) in MeOH (2 mL) and water (0.2 mL) was added sodium periodate (0.12 g, 0.55 mmol) and the mixture was allowed to stir at room temperature for 1 hour. The reaction mixture was extracted with ethyl acetate. The organic layers were collected, dried over sodium sulfate, filtered and evaporated followed by purification on a 12 g silica gel column with DCM to 15% MeOH in DCM to provide tert-butyl (S)-4-(7-bromo-8-fluoro-6-formyl-2-((1-methylpyrrolidin-2-yl)methoxy)quinazolin-4-yl)piperazine-1-carboxylate (0.020 g, 10%). LCMS ESI (+) 552.03 m/z (M+Na). ¹H NMR (300 MHz, CDCl₃) δ 10.38 (s, 1H), 8.28 (s, 1H), 4.75 (m, 1H), 4.61 (m, 1H), 3.97 (m, 4H), 3.70 (m, 4H), 3.51 (m, 1H), 3.33-3.12 (m, 1H), 2.80 (s, 3H), 2.78-2.54 (m, 1H), 2.19 (m, 1H), 2.06 (m, 3H), 1.52 (s, 9H).

Step B: Preparation of tert-butyl (S)-4-(7-bromo-6-(difluoromethyl)-8-fluoro-2-((1-methylpyrrolidin-2-yl)methoxy)quinazolin-4-yl)piperazine-1-carboxylate: To a solution of tert-butyl (S)-4-(7-bromo-8-fluoro-6-formyl-2-((1-methylpyrrolidin-2-yl)methoxy)quinazolin-4-yl)piperazine-1-carboxylate (0.090 g, 0.158 mmol) in DCM (0.50 mL) was added diethylaminosulfur trifluoride (DAST) (0.063 mL, 0.48 mmol) and toluene (0.50 mL). The mixture was heated at 65° C. for 2 hours. After cooling to ambient temperature, the reaction was extracted with DCM-water, the DCM collected and dried with sodium sulfate, filtered, and evaporated. The compound was purified on a 12 g silica column with DCM to 30% MeOH in DCM to provide tert-butyl (S)-4-(7-bromo-6-(difluoromethyl)-8-fluoro-2-((1-methylpyrrolidin-2-yl)methoxy)quinazolin-4-yl)piperazine-1-carboxylate (0.040 g, 44%). LCMS ESI (+) 574.3 m/z (M+H). ¹H NMR (300 MHz, CDCl₃) δ 7.90 (s, 1H), 6.97 (t, J=55.0 Hz, 1H), 4.69 (m, 1H), 4.54 (m, 1H), 3.87 (m, 4H), 3.68 (m, 4H), 3.38 (m, 1H), 3.27-2.93 (m, 1H), 2.72 (s, 3H), 2.63-2.43 (m, 1H), 2.21 (s, 1H), 1.96 (m, 3H), 1.51 (s, 9H).

Step C: Preparation of tert-butyl 4-(7-(2-((tert-butoxycarbonyl)amino)-7-fluorobenzo[d]thiazol-4-yl)-6-(difluoromethyl)-8-fluoro-2-(((S)-1-methylpyrrolidin-2-yl)methoxy)quinazolin-4-yl)piperazine-1-carboxylate: To a mixture of tert-butyl (S)-4-(7-bromo-6-(difluoromethyl)-8-fluoro-2-((1-methylpyrrolidin-2-yl)methoxy)quinazolin-4-yl)piperazine-1-carboxylate (0.04 g, 0.069 mmol), [2-(tert-butoxycarbonylamino)-7-fluoro-1,3-benzothiazol-4-yl]boronic acid (0.043 g, 0.14 mmol) and potassium phosphate (0.019 g, 0.14 mmol) in 1,4-dioxane (0.90 mL) and water (0.27 mL) under argon was added Pd(dtbpf)Cl₂ (0.0069 g, 0.010 mmol) and the mixture heated at 90° C. for 2 hours. The reaction mixture was extracted with ethyl acetate/water. The organic layers were collected, dried over sodium sulfate, filtered, and evaporated. The mixture was purified on a 25 g gold silica gel column with DCM to 15% MeOH in DCM to provide tert-butyl 4-(7-(2-((tert-butoxycarbonyl)amino)-7-fluorobenzo[d]thiazol-4-yl)-6-(difluoromethyl)-8-fluoro-2-(((S)-1-methylpyrrolidin-2-yl)methoxy)quinazolin-4-yl)piperazine-1-carboxylate (0.018 g, 33%). LCMS ESI (+) 784.35 m/z (M+Na). ¹HNMR (300 MHz, CDCl₃) δ 8.01 (s, 1H), 7.35 (m, 1H), 7.22-7.03 (m, 1H), 6.41 (t, J=57 Hz, 1H), 4.69-4.51 (m, 1H), 4.47-4.23 (m, 1H), 3.87 (m, 4H), 3.68 (m, 4H), 3.36-3.08 (m, 1H), 2.97-2.69 (m, 1H), 2.56 (s, 3H), 2.44-2.29 (m, 1H), 2.06 (m, 1H), 1.81 (m, 3H), 1.56 (s, 9H), 1.53 (s, 9H).

Step D: Preparation of 4-(6-(difluoromethyl)-8-fluoro-2-(((S)-1-methylpyrrolidin-2-yl)methoxy)-4-(piperazin-1-yl)quinazolin-7-yl)-7-fluorobenzo[d]thiazol-2-amine: To a solution of tert-butyl 4-(7-(2-((tert-butoxycarbonyl)amino)-7-fluorobenzo[d]thiazol-4-yl)-6-(difluoromethyl)-8-fluoro-2-(((S)-1-methylpyrrolidin-2-yl)methoxy)quinazolin-4-yl)piperazine-1-carboxylate (0.018 g, 0.023 mmol) in DCM (10 mL) was added an excess of TFA. After completion the reaction mixture was evaporated to provide 4-(6-(difluoromethyl)-8-fluoro-2-(((S)-1-methylpyrrolidin-2-yl)methoxy)-4-(piperazin-1-yl)quinazolin-7-yl)-7-fluorobenzo[d]thiazol-2-amine (0.020 g) as TFA salt. LCMS ESI (+) m/z 562.19 (M+H). ¹H NMR (300 MHz, CD₃OD) δ 8.15 (s, 1H), 7.36 (m, 1H), 7.15 (t, J=8.7 Hz, 1H), 6.67 (t, J=54.6 Hz, 1H), 4.95-4.88 (m, 1H), 4.75 (m, 1H), 4.23 (m, 4H), 4.19-4.09 (m, 1H), 3.94 (m, 1H), 3.77 (m, 1H), 3.54 (m, 4H), 3.12 (s, 3H), 2.45 (m, 1H), 2.19 (m, 3H).

### Synthetic Example 27: Synthesis of 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 219), 4-((S)-4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 245) and 4-((R)-4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 246)

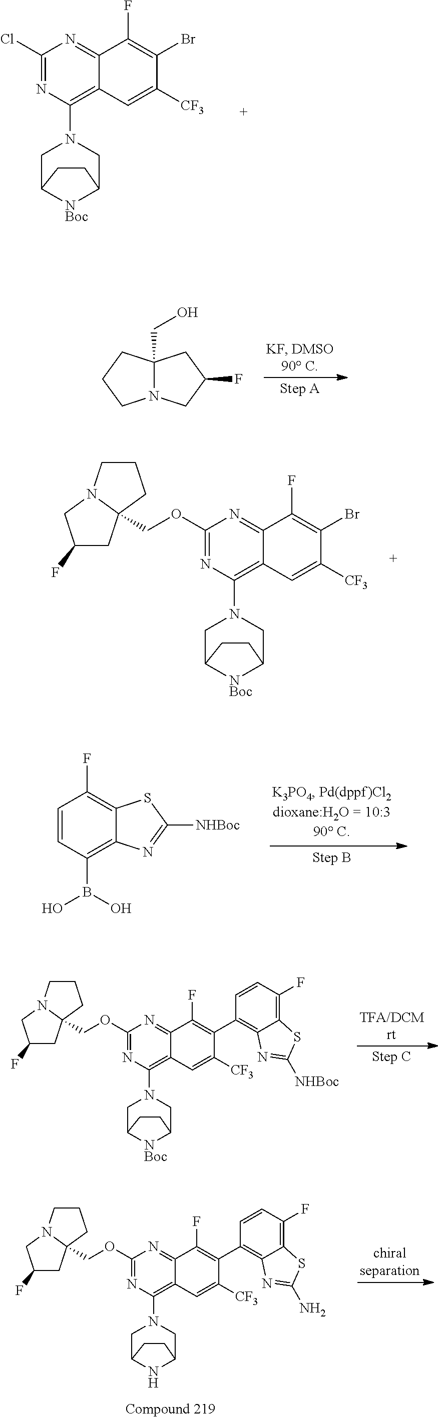 [SMILES: CC(C)(C)OC(=O)N1C2CCC1CN(c1nc(Cl)nc3c(F)c(Br)c(C(F)(F)F)cc13)C2 ; OC[C@@]12CCCN1C[C@H](F)C2 ; CC(C)(C)OC(=O)N1C2CCC1CN(c1nc(OC[C@@]34CCCN3C[C@H](F)C4)nc3c(F)c(Br)c(C(F)(F)F)cc13)C2 ; CC(C)(C)OC(=O)Nc1nc2c(B(O)O)ccc(F)c2s1 ; CC(C)(C)OC(=O)Nc1nc2c(-c3c(C(F)(F)F)cc4c(N5CC6CCC(C5)N6C(=O)OC(C)(C)C)nc(OC[C@@]56CCCN5C[C@H](F)C6)nc4c3F)ccc(F)c2s1 ; Nc1nc2c(-c3c(C(F)(F)F)cc4c(N5CC6CCC(C5)N6)nc(OC[C@@]56CCCN5C[C@H](F)C6)nc4c3F)ccc(F)c2s1]

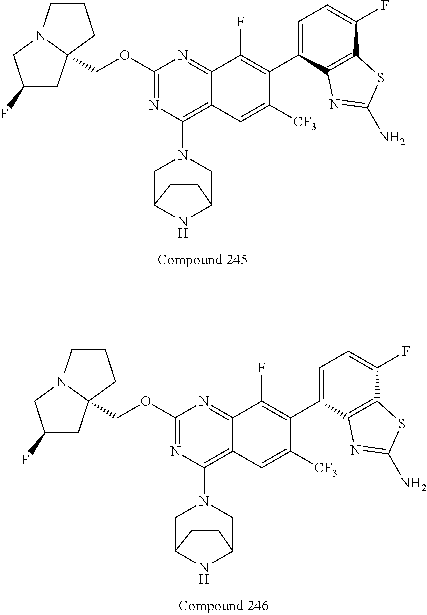

Step A: Preparation of tert-butyl 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate: To a solution of tert-butyl 3-[7-bromo-2-chloro-8-fluoro-6-(trifluoromethyl)quinazolin-4-yl]-3,8-diazabicyclo[3.2.1]octane-8-carboxylate (450 mg, 0.83 mmol) in DMSO (5 mL) was added [(2R,8S)-2-fluoro-1,2,3,5,6,7-hexahydropyrrolizin-8-yl]methanol (159 mg, 1.00 mmol) and KF (388 mg, 6.67 mmol) at ambient temperature. The mixture was stirred for 6 hours at 90° C. After cooling to ambient temperature, ethyl acetate and brine were added. The organic layers were then separated and dried over Na₂SO₄, and filtered before concentration to dryness. The crude product was purified by column chromatography on silica gel, eluting with 50% EtOAc in petroleum ether, to give tert-butyl 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (245 mg, 44%). LCMS (ES+) m/z 662.2 (M+H).

Step B: Preparation of tert-butyl-3-(7-(2-((tert-butoxycarbonyl)amino)-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)-3,8-diazabicyclo[3.2.1]octane-8-carboxylate: To a mixture of tert-butyl 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (40 mg, 0.060 mmol) in water (0.2 mL) and 1,4-dioxane (1 mL) was added [2-(tert-butoxycarbonylamino)-7-fluoro-1,3-benzothiazol-4-yl]boronic acid (21 mg, 0.066 mmol), 1,1′-bis(di-t-butylphosphino)ferrocene palladium dichloride (3.9 mg, 0.0060 mmol) and potassium phosphate (26 mg, 0.12 mmol) at ambient temperature. The mixture was bubbled with argon for 1-2 minutes and then sealed. After that, the mixture was stirred for 1 hour at 90° C. After cooling to ambient temperature, the reaction was concentrated to dryness and the residue was taken up in EtOAc (20 mL) and the organic layers washed with saturated brine solution. The organic layers were then separated, dried (MgSO₄), filtered, and concentrated under reduced pressure. The crude was then purified by preparative-TLC (DCM/MeOH=20/1) to give tert-butyl 3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (44 mg, 86%). LCMS (ES+) m/z 850.3 (M+H).

Step C: Preparation of 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 219), 4-((S)-4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 245) and 4-((R)-4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 246): To a solution of tert-butyl 3-[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (44 mg, 0.052 mmol) in DCM (3 mL) was added TFA (1.5 mL, 19.5 mmol) at ambient temperature and stirred at this temperature for 2 hours. The reaction was concentrated under reduced pressure. The residue obtained was then purified by preparative RP-HPLC to give got the crude product of 4-[4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-1,3-benzothiazol-2-amine (26 mg, 75%). LCMS ESI (+) m/z 650.2 (M+H). ¹HNMR (400 MHz, CD₃OD) δ 8.18 (s, 1H), 7.17-7.27 (m, 1H), 6.98-7.09 (m, 1H), 5.38-5.67 (m, 1H), 4.65-4.77 (m, 3H), 4.22-4.31 (m, 2H), 3.82-4.10 (m, 6H), 3.45-3.53 (m, 1H), 2.58-2.75 (m, 2H), 2.32-2.47 (m, 3H), 2.05-2.22 (m, 5H). Separated 4-[4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-1,3-benzothiazol-2-amine (Compound 219) (133.2 mg) with chiral chromatography condition (ChiralPak IE 3 cm×25 cm, 5 uM, MeOH:DCM=50:50 (0.2% 2 mM ammonium in methanol), 25 mL/min) to give one atropisomer, 4-((S)-4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 245) (41 mg, 84% de), as second peak; LCMS ESI (+) m/z 650.3 (M+H). ¹HNMR (400 MHz, CD₃OD) δ 8.14 (s, 1H), 7.17-7.20 (m, 1H), 6.94-6.99 (m, 1H), 5.32 and 5.45 (m, 1H), 4.59-4.84 (m, 2H), 4.36-4.55 (m, 2H), 3.73-3.84 (m, 4H), 3.39-3.76 (m, 3H), 3.14-3.20 (m, 1H), 1.87-2.25 (m, 10H); and give the other atropisomer, 4-((R)-4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 246) (46 mg, 98% de), as first peak. LCMS ESI (+) m/z 650.3 (M+H). ¹HNMR (400 MHz, CD₃OD) δ 8.16 (s, 1H), 7.17-7.21 (m, 1H), 6.96-7.00 (m, 1H), 5.43 and 5.56 (m, 1H), 4.62-4.74 (m, 2H), 4.54-4.59 (m, 2H), 4.09 (bs, 2H), 3.83-3.91 (m, 2H), 3.66-3.73 (m, 2H), 3.35 (m, 2H), 1.87-2.25 (m, 10H).

### Synthetic Example 28: Synthesis of 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]oxazol-2-amine (Compound 220)

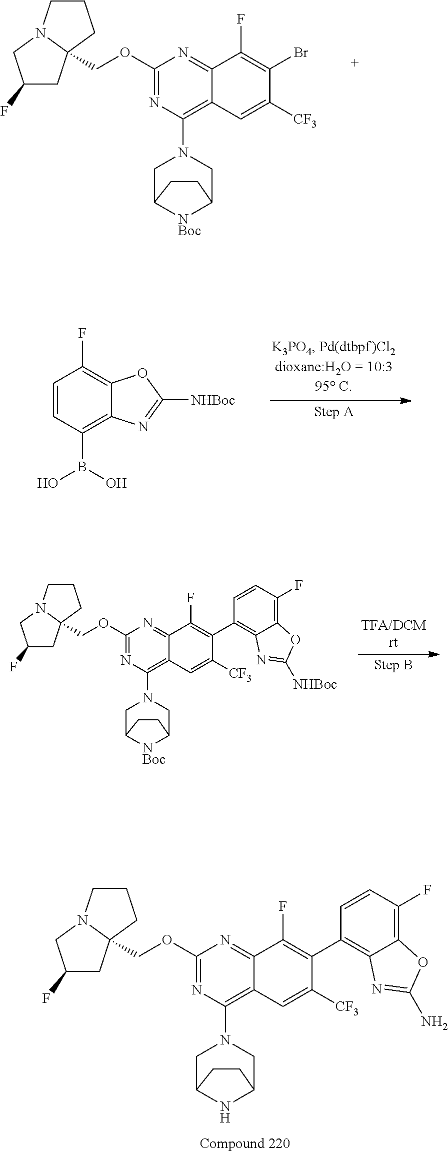 [SMILES: CC(C)(C)OC(=O)N1C2CCC1CN(c1nc(OC[C@@]34CCCN3C[C@H](F)C4)nc3c(F)c(Br)c(C(F)(F)F)cc13)C2 ; CC(C)(C)OC(=O)Nc1nc2c(B(O)O)ccc(F)c2o1 ; CC(C)(C)OC(=O)Nc1nc2c(-c3c(C(F)(F)F)cc4c(N5CC6CCC(C5)N6C(=O)OC(C)(C)C)nc(OC[C@@]56CCCN5C[C@H](F)C6)nc4c3F)ccc(F)c2o1 ; Nc1nc2c(-c3c(C(F)(F)F)cc4c(N5CC6CCC(C5)N6)nc(OC[C@@]56CCCN5C[C@H](F)C6)nc4c3F)ccc(F)c2o1]

Step A: Preparation of tert-butyl 3-[7-(2-amino-7-fluoro-1,3-benzoxazol-4-yl)-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate: To a mixture of tert-butyl 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (57 mg, 0.086 mmol) in water (0.3 mL) and 1,4-dioxane (1.5 mL) was added (2-((tert-butoxycarbonyl)amino)-7-fluorobenzo[d]oxazol-4-yl)boronic acid (25 mg, 0.13 mmol), 1,1′-bis (di-t-butylphosphino)ferrocene palladium dichloride (5.6 mg, 0.0086 mmol) and potassium phosphate (54 mg, 0.26 mmol) at ambient temperature. The mixture was bubbled with argon for about 1-2 minutes and then sealed. After that, the mixture was stirred for 1 hour at 90° C. After cooling to ambient temperature, the reaction was concentrated to dryness and the residue was taken up in EtOAc (20 mL) and the organic layers washed with saturated brine solution. The organic layers were then separated, dried (MgSO₄), filtered, and concentrated under reduced pressure. The crude was then purified by preparative-TLC (DCM/MeOH=20/1) to give tert-butyl 3-[7-(2-amino-7-fluoro-1,3-benzoxazol-4-yl)-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (22 mg, 35%).

Step B: Preparation of 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]oxazol-2-amine: To a solution of tert-butyl 3-[7-(2-amino-7-fluoro-1,3-benzoxazol-4-yl)-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (1.00 eq, 22 mg, 0.0300 mmol) in DCM (3 mL) was added TFA (1.5 mL, 19.5 mmol) at ambient temperature and stirred at this temperature for 2 hours. The reaction was concentrated under reduced pressure. The residue obtained was then purified by preparative RP-HPLC to give 4-[4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-1,3-benzoxazol-2-amine (13 mg, 65%). LCMS ESI (+) m/z 634.2 (M+H). ¹HNMR (400 MHz, CD₃OD) δ 8.17 (s, 1H), 7.05-7.12 (m, 1H), 6.96-7.03 (m, 1H), 5.50-5.64 (m, 1H), 4.67-4.82 (m, 3H), 4.23-4.31 (m, 2H), 3.83-4.09 (m, 6H), 3.45-3.55 (m, 1H), 2.56-2.77 (m, 2H), 2.32-2.45 (m, 3H), 2.07-2.23 (m, 5H).

### Synthetic Example 29: Synthesis of 7-fluoro-4-(8-fluoro-4-(piperazin-1-yl)-2-((tetrahydro-1H-pyrrolizin-7a(5H)-yl)methoxy)-6-(trifluoromethoxy)quinazolin-7-yl)benzo[d]thiazol-2-amine (Compound 228)

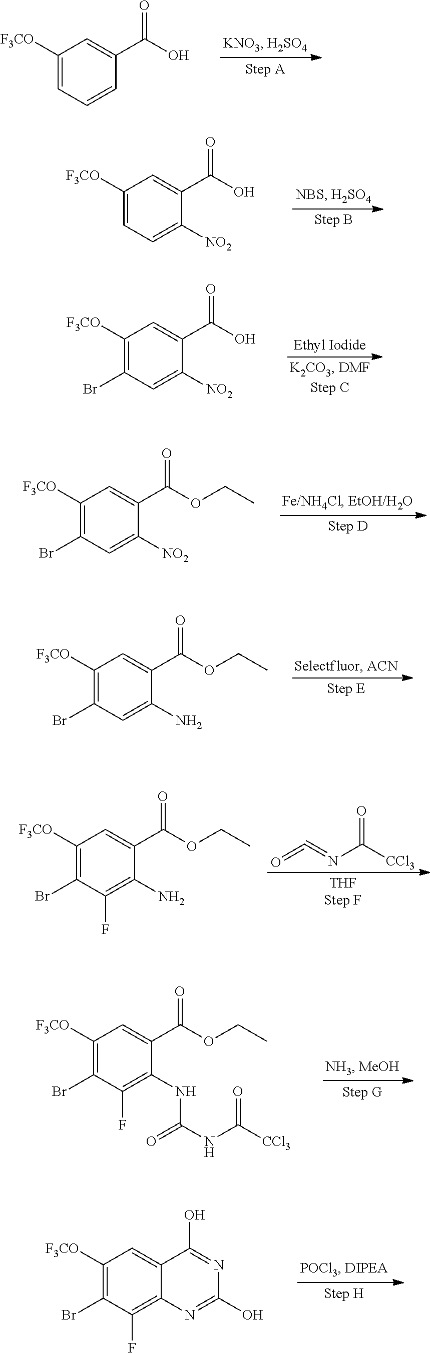

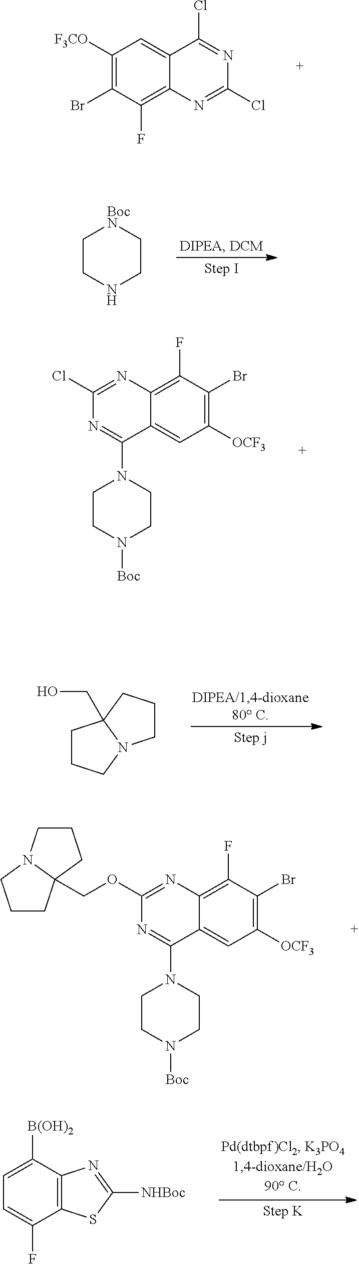 [SMILES: Fc1c(Br)c(OC(F)(F)F)cc2c(Cl)nc(Cl)nc12 ; CC(C)(C)OC(=O)N1CCNCC1 ; CC(C)(C)OC(=O)N1CCN(c2nc(Cl)nc3c(F)c(Br)c(OC(F)(F)F)cc23)CC1 ; OCC12CCCN1CCC2 ; CC(C)(C)OC(=O)N1CCN(c2nc(OCC34CCCN3CCC4)nc3c(F)c(Br)c(OC(F)(F)F)cc23)CC1 ; CC(C)(C)OC(=O)Nc1nc2c(B(O)O)ccc(F)c2s1]

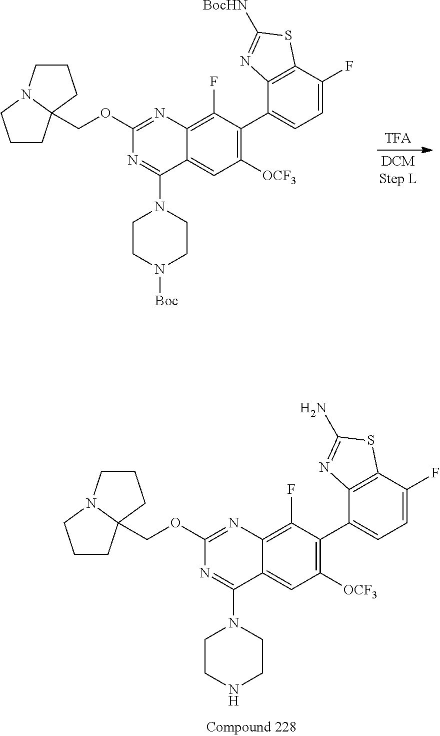 [SMILES: CC(C)(C)OC(=O)Nc1nc2c(-c3c(OC(F)(F)F)cc4c(N5CCN(C(=O)OC(C)(C)C)CC5)nc(OCC56CCCN5CCC6)nc4c3F)ccc(F)c2s1 ; Nc1nc2c(-c3c(OC(F)(F)F)cc4c(N5CCNCC5)nc(OCC56CCCN5CCC6)nc4c3F)ccc(F)c2s1]

Step A: Preparation of 2-nitro-5-(trifluoromethoxy)benzoic acid: A stirred solution of 3-(trifluoromethoxy)benzoic acid (30.0 g, 146 mmol) in sulfuric acid (300 mL) was cooled to 0° C. and was added dropwise potassium nitrate (44.1 g, 437 mmol) dissolved in sulfuric acid (180 mL). After addition, the reaction mixture was stirred at ambient temperature for 18 hours. The reaction mixture was quenched with ice and the reaction mixture was extracted with ethyl acetate. The combined organic layers were washed with brine, dried over sodium sulfate, filtered, and concentrated under reduced pressure. The resulting solid was triturated with hexane to afford 2-nitro-5-(trifluoromethoxy)benzoic acid. LCMS ESI (−) m/z 250.98 (M−H). ¹H NMR (300 MHz, CDCl₃) δ 9.44 (s, 1H), 8.00 (d, J=8.9 Hz, 1H), 7.68 (d, J=2.7 Hz, 1H), 7.52 (d, J=7.9 Hz, 1H).

Step B: Preparation of 4-bromo-2-nitro-5-(trifluoromethoxy)benzoic acid: A stirred solution of 2-nitro-5-(trifluoromethoxy)benzoic acid (5.00 g, 19.9 mmol) in sulphuric acid (10 mL, 187 mmol) was heated to 80° C. followed by the addition of N-bromosuccinimide (3.54 g, 19.9 mmol) in 3 portions at 10 minutes intervals. After addition, the reaction mixture was stirred at 80° C. for 2.5 hours and additional N-bromosuccinimide (1.77 g, 9.96 mmol) was added and stirred for another 3 hours at 80° C. After cooling to ambient temperature, the reaction mixture was quenched with ice cold water and stirred for 15 minutes. The resulting solid was collected by filtration, washed with water, and dried to afford 4-bromo-2-nitro-5-(trifluoromethoxy) benzoic acid (2.53 g, 38%). ¹HNMR (300 MHz, CDCl₃) δ 8.21 (s, 1H), 7.80 (d, J=1.2 Hz, 1H).

Step C: Preparation of ethyl 4-bromo-2-nitro-5-(trifluoromethoxy)benzoate: To a stirred solution of 4-bromo-2-nitro-5-(trifluoromethoxy)benzoic acid (12.0 g, 36.4 mmol) in DMF (120 mL) were added potassium carbonate (15.08 g, 109.0 mmol) and ethyl iodide (3.50 mL, 43.6 mmol). The reaction mixture was stirred for 1 hour at ambient temperature. The mixture was diluted with ethyl acetate, washed with brine, dried over sodium sulfate, filtered, and concentrated under reduced pressure. The residue was purified by chromatography on silica gel by eluting with 0-100% hexane/ethyl acetate to afford ethyl 4-bromo-2-nitro-5-(trifluoromethoxy)benzoate (9.10 g, 69%). LCMS ESI (−) m/z 356.99 (M−H). ¹H NMR (300 MHz, CDCl₃) δ 8.22 (s, 1H), 7.68 (d, J=1.3 Hz, 1H), 4.43 (q, J=7.1 Hz, 2H), 1.39 (t, J=7.1 Hz, 3H).

Step D: Preparation of ethyl 2-amino-4-bromo-5-(trifluoromethoxy)benzoate: To a stirred solution of ethyl 4-bromo-2-nitro-5-(trifluoromethoxy)benzoate (5.40 g, 15.1 mmol) in ethanol (50 mL) were added iron (2.52 g, 45.2 mmol) and NH₄C₁ saturated solution (10 mL) at ambient temperature. The reaction mixture was stirred at reflux for 16 hours. After the reaction was cooled to ambient temperature, and solvent removed under reduced pressure. The mixture was diluted with ethyl acetate and filtered through Celite®. The filtrate was washed with water followed by brine, dried over sodium sulfate, filtered, and concentrated under reduced pressure. The residue was purified on flash chromatography on silica gel by eluting with 0-100% hexane/ethyl acetate to afford ethyl 2-amino-4-bromo-5-(trifluoromethoxy)benzoate (3.60 g, 72%). LCMS ESI (−) m/z 328.05 (M−H). ¹H NMR (300 MHz, CDCl₃) δ 7.77 (s, 1H), 6.93 (s, 1H), 5.83 (s, 2H), 4.42-4.22 (m, 2H), 1.38 (t, J=7.1 Hz, 3H).

Step E: Preparation of ethyl 2-amino-4-bromo-3-fluoro-5-(trifluoromethoxy)benzoate: To a mixture of ethyl 2-amino-4-bromo-5-(trifluoromethoxy)benzoate (5.00 g, 15.2 mmol) in acetonitrile (12 mL) was added selectfluor (8.10 g, 22.9 mmol) and stirred at room temperature for 6 hours. The reaction mixture was diluted with ethyl acetate and washed with water. The ethyl acetate was collected, dried over sodium sulfate, filtered, and evaporated under reduced pressure. The crude material was purified on 220 g silica gel column by eluting with 0-15% hexane/ethyl acetate to afford ethyl 2-amino-4-bromo-3-fluoro-5-(trifluoromethoxy)benzoate (0.80 g, 15%). LCMS ESI (+) m/z 345.94 (M+H). ¹H NMR (300 MHz, CDCl₃) δ 7.65 (s, 1H), 5.96 (s, 2H), 4.37 (q, J=7.1 Hz, 2H), 1.40 (t, J=7.1 Hz, 3H).

Step F: Preparation of ethyl 4-bromo-3-fluoro-2-(3-(2,2,2-trichloroacetyl)ureido)-5-(trifluoromethoxy)benzoate: To a stirred solution of ethyl 2-amino-4-bromo-3-fluoro-5-(trifluoromethoxy)benzoate (0.80 g, 2.31 mmol) in THE (10 mL) was added 2,2,2-trichloroacetyl isocyanate (0.41 mL, 3.47 mmol) and stirred at ambient temperature for 30 minutes. The solvent was removed under reduced pressure, triturated with MTBE followed by filtration to afford ethyl 4-bromo-3-fluoro-2-[(2,2,2-trichloroacetyl)carbamoylamino]-5-(trifluoromethoxy)benzoate (1.20 g, 97%). ¹HNMR (300 MHz, CDCl₃) δ 10.61 (s, 1H), 8.65 (s, 1H), 7.83 (s, 1H), 4.48 (q, J=7.2 Hz, 2H), 1.45 (t, J=7.2 Hz, 3H).

Step G: Preparation of 7-bromo-8-fluoro-6-(trifluoromethoxy)quinazoline-2,4-diol: To a stirred solution of methyl 4-bromo-3-fluoro-2-[(2,2,2-trichloroacetyl)carbamoylamino]-5-(trifluoromethoxy)benzoate (1.20 g, 2.31 mmol) in methanol (1 mL) was added a 7 N ammonia solution in methanol (0.76 mL, 5.30 mmol) and stirred for 30 minutes at ambient temperature. The reaction mixture was concentrated under reduced pressure and co-distilled with methanol two times followed by one time with MTBE. The residue was triturated with MTBE, filtered and dried to afford 7-bromo-8-fluoro-6-(trifluoromethoxy)quinazoline-2,4-diol (0.78 g, 98%). LCMS ESI (−) m/z 341.03 (M−H). 1H NMR (300 MHz, d6-DMSO) δ 7.63 (s, 1H).

Step H: Preparation of 7-bromo-2,4-dichloro-8-fluoro-6-(trifluoromethoxy)quinazoline: To a stirred solution of 7-bromo-8-fluoro-6-(trifluoromethoxy)quinazoline-2,4-diol (162 mg, 0.47 mmol) in POCl₃ (1.00 mL, 10.9 mmol) at 0° C. was added DIPEA (0.16 mL, 0.95 mmol). The reaction mixture was stirred at room temperature for 15 minutes then at 110° C. for an hour. After cooling to ambient temperature, the solvent was evaporated, and the residue was dissolved in ethyl acetate (10 mL). The organic layer was washed with a saturated NaHCO₃ solution, dried over Na₂SO₄, filtered, concentrated under reduced pressure, and co-distilled with DCM three times to afford 7-bromo-2,4-dichloro-8-fluoro-6-(trifluoromethoxy)quinazoline as a crude product (0.50 g, 99%). LCMS ESI (+) m/z 378.82 (M+H). ¹H NMR (300 MHz, CDCl₃) δ 7.97 (s, 1H).

Step I: Preparation of tert-butyl 4-(7-bromo-2-chloro-8-fluoro-6-(trifluoromethoxy)quinazolin-4-yl)piperazine-1-carboxylate: To a stirred solution of 7-bromo-2,4-dichloro-8-fluoro-6-(trifluoromethoxy)quinazoline (0.50 g, 1.32 mmol) in DCM (5 mL) was added DIPEA (0.69 mL, 3.95 mmol) at −40° C. followed by 1-Boc piperazine (0.29 g, 1.58 mmol). After addition, the mixture was warmed to ambient temperature and stirred at ambient temperature for 15 minutes. The mixture was diluted with DCM, the organic layer was washed with a saturated aqueous solution of NaHCO₃, dried over Na₂SO₄, filtered, and concentrated under reduced pressure. The crude material was purified on silica gel column by eluting with 0-100% hexane/ethyl acetate to afford tert-butyl 4-(7-bromo-2-chloro-8-fluoro-6-(trifluoromethoxy)quinazolin-4-yl]piperazine-1-carboxylate (500 mg, 71.72% yield). LCMS ESI (+) m/z 551.04 (M+H). ¹HNMR (300 MHz, CDCl₃) δ 7.56 (s, 1H), 3.91-3.77 (m, 4H), 3.71-3.52 (m, 4H), 1.49 (s, 9H).

Step J: Preparation of tert-butyl 4-(7-bromo-8-fluoro-2-((tetrahydro-1H-pyrrolizin-7a(5H)-yl)methoxy)-6-(trifluoromethoxy)quinazolin-4-yl)piperazine-1-carboxylate: To a stirred solution of tert-butyl 4-[7-bromo-2-chloro-8-fluoro-6-(trifluoromethoxy)quinazolin-4-yl]piperazine-1-carboxylate (0.21 g, 0.39 mmol) in 1,4-dioxane (3.2 mL) was added cesium carbonate (0.25 g, 0.77 mmol) and a solution of 1,2,3,5,6,7-hexahydropyrrolizin-8-ylmethanol (0.11 mL, 0.77 mmol) in 0.3 mL 1,4-dioxane at 10° C. Then the reaction mixture was heated to 80° C. and stirred for 16 hours. The reaction mixture was cooled to ambient temperature and diluted with ethyl acetate. The organic layer was washed with water then dried over Na₂SO₄, filtered, and concentrated under reduced pressure. The crude material was purified on silica gel column by eluting with 0-30% DCM/MeOH to afford tert-butyl 4-[7-bromo-8-fluoro-2-(1,2,3,5,6,7-hexahydropyrrolizin-8-ylmethoxy)-6-(trifluoromethoxy)quinazolin-4-yl]piperazine-1-carboxylate (0.095 g, 38%). LCMS ESI (+) m/z 643.31 (M+H). ¹H NMR (300 MHz, CDCl₃) δ 7.53 (s, 1H), 4.28 (s, 2H), 3.85-3.72 (m, 4H), 3.72-3.59 (m, 4H), 3.30-3.08 (m, 2H), 2.84-2.60 (m, 2H), 2.21-2.07 (m, 2H), 2.04-1.88 (m, 4H), 1.84-1.66 (m, 2H), 1.51 (s, 9H).

Step K: Preparation of tert-butyl 4-(7-(2-((tert-butoxycarbonyl)amino)-7-fluorobenzo[d]thiazol-4-yl)-8-fluoro-2-((tetrahydro-1H-pyrrolizin-7a(5H)-yl)methoxy)-6-(trifluoromethoxy)quinazolin-4-yl)piperazine-1-carboxylate: To a stirred solution of tert-butyl 4-[7-bromo-8-fluoro-2-(1,2,3,5,6,7-hexahydropyrrolizin-8-ylmethoxy)-6-(trifluoromethoxy)quinazolin-4-yl]piperazine-1-carboxylate (0.044 g, 0.069 mmol) in 1,4-dioxane (0.9 mL) and water (0.27 mL) were added [2-(tert-butoxycarbonylamino)-7-fluoro-1,3-benzothiazol-4-yl]boronic acid (0.043 g, 0.14 mmol) and potassium phosphate (0.019 g, 0.14 mmol). The mixture was purged with argon for 5 minutes followed by the addition of 1,1′-bis(di-t-butylphosphino)ferrocene palladium dichloride (0.0068 g, 0.010 mmol) at ambient temperature. Then the reaction mixture was stirred at 90° C. for 2 hours. The reaction mixture was cooled to ambient temperature and diluted with ethyl acetate. The organic layer was washed with water, dried over Na₂SO₄, filtered, and concentrated under reduced pressure. The crude material was purified on silica gel column by eluting with 0-30% DCM/MeOH to afford tert-butyl 4-[7-[2-(tert-butoxycarbonylamino)-7-fluoro-1,3-benzothiazol-4-yl]-8-fluoro-2-(1,2,3,5,6,7-hexahydropyrrolizin-8-ylmethoxy)-6-(trifluoromethoxy)quinazolin-4-yl]piperazine-1-carboxylate (0.010 g, 17%). LCMS ESI (+) m/z 822.14 (M+H). ¹H NMR (300 MHz, CDCl₃) δ 7.54 (s, 1H), 7.33 (dd, J=8.2, 5.1 Hz, 1H), 7.08 (t, J=8.6 Hz, 1H), 4.60-4.43 (m, 2H), 3.89-3.73 (m, 4H), 3.72-3.58 (m, 4H), 3.58-3.47 (m, 2H), 2.86-2.71 (m, 2H), 2.34-2.15 (m, 2H), 2.12-1.92 (m, 4H), 1.89-1.70 (m, 2H), 1.53 (s, 9H), 1.49 (s, 9H).

Step L: Preparation of 7-fluoro-4-(8-fluoro-4-(piperazin-1-yl)-2-((tetrahydro-1H-pyrrolizin-7a(5H)-yl)methoxy)-6-(trifluoromethoxy)quinazolin-7-yl)benzo[d]thiazol-2-amine: To a stirring solution tert-butyl 4-[7-[2-(tert-butoxycarbonylamino)-7-fluoro-1,3-benzothiazol-4-yl]-8-fluoro-2-(1,2,3,5,6,7-hexahydropyrrolizin-8-ylmethoxy)-6-(trifluoromethoxy)quinazolin-4-yl]piperazine-1-carboxylate (0.010 g, 0.012 mmol) in DCM (1 mL) was added trifluoroacetic acid (0.085 mL, 1.10 mmol) at ambient temperature and stirred at this temperature for 2 hours. The solvent was evaporated and co-distilled with DCM multiple times and dried under high vacuum to afford 7-fluoro-4-[8-fluoro-2-(1,2,3,5,6,7-hexahydropyrrolizin-8-ylmethoxy)-4-piperazin-1-yl-6-(trifluoromethoxy)quinazolin-7-yl]-1,3-benzothiazol-2-amine (0.011 g, 96%). LCMS ESI (+) m/z 622.36 (M+H). ¹H NMR (300 MHz, CD₃OD) δ 7.68 (s, 1H), 7.24 (dd, J=8.4, 5.1 Hz, 1H), 7.01 (t, J=8.8 Hz, 1H), 4.54 (s, 2H), 4.08-3.96 (m, 4H), 3.63-3.49 (m, 2H), 3.41-3.34 (m, 2H), 3.22-3.07 (m, 4H), 2.20-1.91 (m, 8H).

### Synthetic Example 30: 4-(6-(difluoromethoxy)-8-fluoro-4-(piperazin-1-yl)-2-((tetrahydro-1H-pyrrolizin-7a(5H)-yl)methoxy)quinazolin-7-yl)-7-fluorobenzo[d]thiazol-2-amine (Compound 234)

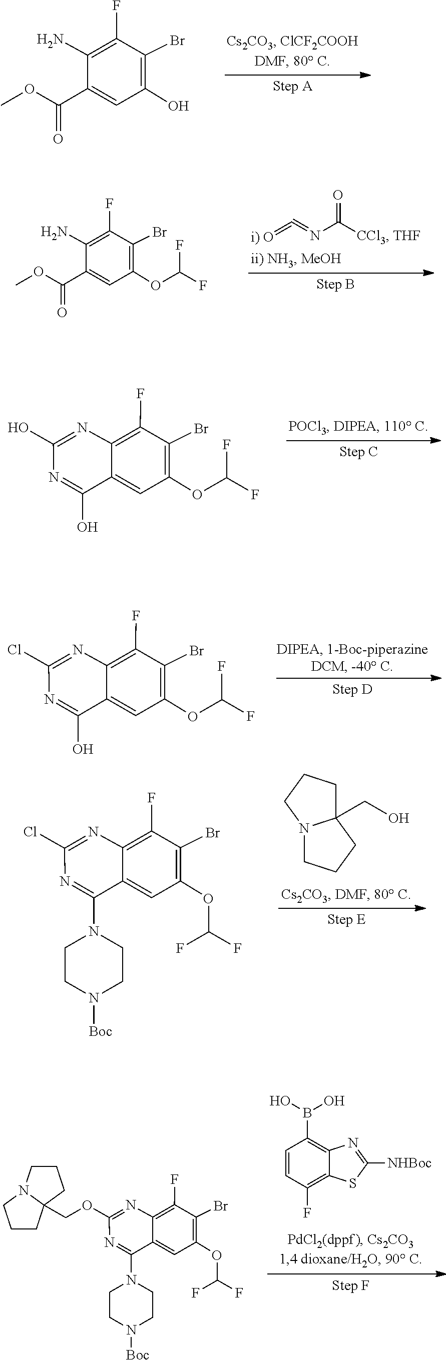

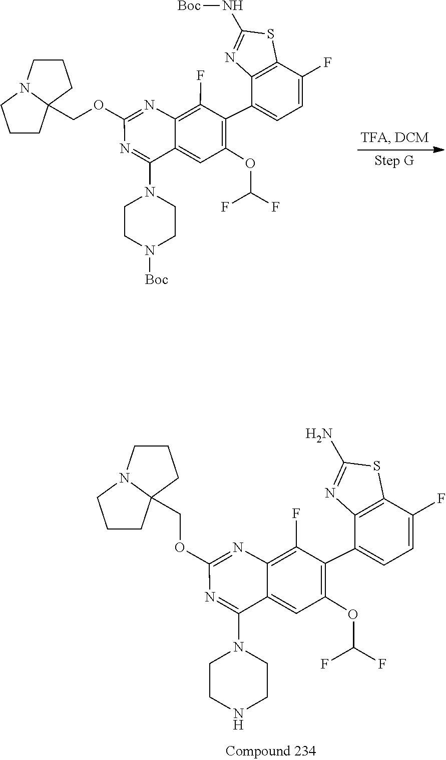 [SMILES: CC(C)(C)OC(=O)Nc1nc2c(-c3c(OC(F)F)cc4c(N5CCN(C(=O)OC(C)(C)C)CC5)nc(OCC56CCCN5CCC6)nc4c3F)ccc(F)c2s1 ; Nc1nc2c(-c3c(OC(F)F)cc4c(N5CCNCC5)nc(OCC56CCCN5CCC6)nc4c3F)ccc(F)c2s1]

Step A: Preparation of methyl 2-amino-4-bromo-5-(difluoromethoxy)-3-fluorobenzoate: To a stirred solution of methyl 2-amino-4-bromo-3-fluoro-5-hydroxy-benzoate (0.53 g, 2.01 mmol) in DMF (10.6 mL), Cs₂CO₃ (0.196 g, 6.02 mmol) followed by chlorodifluoroacetic acid (340 μL, 4.01 mmol) were added at room temperature under argon atmosphere. The reaction mixture was heated at 80° C. After 17 hours of heating, the reaction mixture was cooled to room temperature and poured into ice water and EtOAc and stirred for 5 minutes. Then layers were separated, and the aqueous layer was further extracted with EtOAc (2×30 mL). The organic layers were combined, dried over Na₂SO₄, filtered, and concentrated. The resulted crude was purified by flash column chromatography on silica gel (eluted with a gradient of 14-18% EtOAc in hexane) to afford title compound (0.26 g, 41%) as white solid. LRMS (ESI+) m/z 314.14 [M+H]. ¹H NMR (300 MHz, CDCl₃) δ 7.56 (dt, J=2.0, 0.9 Hz, 1H), 6.43 (t, J=73.5 Hz, 1H), 5.88 (br s, 2H), 3.90 (s, 3H).

Step B: Preparation of 7-bromo-6-(difluoromethoxy)-8-fluoroquinazoline-2,4-diol: To a stirring solution of methyl 2-amino-4-bromo-5-(difluoromethoxy)-3-fluoro-benzoate (0.26 g, 0.84 mmol) in THE (1.4 mL), 2,2,2-trichloroacetyl isocyanate (150 μL, 1.26 mmol) was added at room temperature under argon. After 15 minutes of stirring at room temperature, the reaction mixture was concentrated under vacuum to provide a white solid, which was used for further step without any purification. MS (ESI+) m/z 522.95 [M+Na].

To a stirring suspension of above methyl 4-bromo-5-(difluoromethoxy)-3-fluoro-2-(3-(2,2,2-trichloroacetyl)ureido)benzoate (0.42 g, 0.84 mmol) in methanol (5 mL) at room temperature, 7N NH₃ in methanol (0.27 mL, 1.92 mmol) was added and the reaction mixture was stirred vigorously at room temperature for 17 hours. The reaction mixture was concentrated in vacuo. The crude was taken in water/EtOAc, and further the aqueous layer was extracted with EtOAc (2×30 mL). The organic layer was dried over Na₂SO₄, filtered, and concentrated to provide crude material which was used for next step without any purification. LRMS (ESI+) m/z 322.95 [M+H]. ¹HNMR (300 MHz, (CD₃)₂SO) δ 11.62 (br s, 2H), 7.61 (s, 1H), 7.39 (t, J=72.8 Hz, 1H).

Step C: Preparation of 7-bromo-2,4-dichloro-6-(difluoromethoxy)-8-fluoroquinazoline: POCl₃ (1.9 mL, 20.5 mmol) was added to a round bottom flask containing 7-bromo-6-(difluoromethoxy)-8-fluoroquinazoline-2,4-diol (0.29 g, 0.89 mmol) and cooled to 0° C. DIPEA (0.31 mL, 1.78 mmol) was added dropwise under argon atmosphere and stirred at 0° C. for 10 minutes. Then the reaction temperature was stirred at 110° C. for 1 hour. After cooling to ambient temperature, the reaction mixture was concentrated in vacuo and co-distilled with DCM three times to give light yellow-brown color oily residue. This was directly taken to next step without any further purification. ¹HNMR (300 MHz, CDCl₃) δ 7.81 (dt, J=1.9, 0.9 Hz, 1H), 6.79 (t, J=71.3 Hz, 1H).

Step D: Preparation of tert-butyl 4-[7-bromo-2-chloro-6-(difluoromethoxy)-8-fluoroquinazolin-4-yl]piperazine-1-carboxylate: A solution of 7-bromo-2,4-dichloro-6-(difluoromethoxy)-8-fluoroquinazoline (0.32 g, 0.88 mmol) in DCM (4 mL) was cooled at −40° C. under argon. To this, 1-Boc-piperazine (0.20 g, 1.06 mmol) and DIPEA (0.46 mL, 2.65 mmol) were added subsequently. Then the reaction was continued in a same cooling bath for 15 minutes without any external heat. The reaction mixture was diluted with DCM and washed with water (1×20 mL), dried over Na₂SO₄, concentrated under vacuum. The crude was purified by flash column chromatography on silica gel (eluted with gradient of 40-45% EtOAc in hexane) to afford title compound (340 mg, 75%) as a white solid. ¹HNMR (300 MHz, CDCl₃) δ 7.52 (s, 1H), 6.64 (t, J=72.1 Hz, 1H), 4.02-3.29 (m, 8H), 1.50 (s, 9H).

Step E: Preparation of tert-butyl 4-(7-bromo-6-(difluoromethoxy)-8-fluoro-2-((tetrahydro-1H-pyrrolizin-7a(5H)-yl)methoxy)quinazolin-4-yl)piperazine-1-carboxylate: A stirring suspension of tert-butyl 4-[7-bromo-2-chloro-6-(difluoromethoxy)-8-fluoro-quinazolin-4-yl]piperazine-1-carboxylate (130 mg, 0.254 mmol) in 1,4-dioxane (1.5 mL) was cooled to 0° C. and 1,2,3,5,6,7-hexahydropyrrolizin-8-ylmethanol (141 μL, 1.02 mmol) and Cs₂CO₃ (166 mg, 0.508 mmol) were added subsequently. The reaction mixture was stirred at 80° C. for 48 hours. After cooling to ambient temperature, the reaction mixture was concentrated in vacuo to remove the solvent and added water/EtOAc. The organic layer was separated, dried over Na₂SO₄, filtered, and concentrated in vacuo. The resultant crude was purified by flash chromatography on silica gel (eluted with gradient 20-30% of DCM in methanol) to afford tert-butyl 4-(7-bromo-6-(difluoromethoxy)-8-fluoro-2-((tetrahydro-1H-pyrrolizin-7a(5H)-yl)methoxy)quinazolin-4-yl)piperazine-1-carboxylate (23 mg, 15%). ¹HNMR (300 MHz, CDCl₃) δ 7.46 (dd, J=2.0, 1.1 Hz, 1H), 6.60 (t, J=72.8 Hz, 1H), 4.28 (s, 2H), 3.82-3.72 (m, 4H), 3.70-3.59 (m, 4H), 3.23 (dt, J=10.7, 5.3, 4.8 Hz, 2H), 2.69 (dt, J=10.3, 6.8 Hz, 2H), 2.19-2.07 (m, 2H), 1.92 (p, J=6.6 Hz, 4H), 1.71 (dt, J=12.4, 7.4 Hz, 2H), 1.51 (s, 9H).

Step F: Preparation of tert-butyl 4-(7-(2-((tert-butoxycarbonyl)amino)-7-fluorobenzo[d]thiazol-4-yl)-6-(difluoromethoxy)-8-fluoro-2-((tetrahydro-1H-pyrrolizin-7a(5H)-yl)methoxy)quinazolin-4-yl)piperazine-1-carboxylate: To a stirring suspension of tert-butyl 4-(7-bromo-6-(difluoromethoxy)-8-fluoro-2-((tetrahydro-1H-pyrrolizin-7a(5H)-yl)methoxy)quinazolin-4-yl)piperazine-1-carboxylate (14 mg, 0.023 mmol) in 1,4-dioxane (1.5 mL), (2-((tert-butoxycarbonyl)amino)-7-fluorobenzo[d]thiazol-4-yl)boronic acid (14.2 mg, 0.045 mmol) and an aqueous solution of Cs₂CO₃ (14.8 mg, 0.045 mmol) in water (0.15 mL). The reaction mixture was degassed with argon for 10 minutes then Pd(dppf)Cl₂ (2.8 mg, 0.0034 mmol) was added and degassed again for 10 minutes. The reaction mixture was heated to 90° C. for 90 minutes. The reaction mixture was cooled to room temperature and diluted with water/EtOAc. Layers were separated and the aqueous layer was further extracted with EtOAc (2×15 mL). The organic layers were combined, washed with brine, dried over Na₂SO₄, filtered, and concentrated in vacuo. The resultant crude was purified by flash chromatography on silica gel (eluted with 45-50% EtOAc in hexane) to afford tert-butyl 4-(7-(2-((tert-butoxycarbonyl)amino)-7-fluorobenzo[d]thiazol-4-yl)-6-(difluoromethoxy)-8-fluoro-2-((tetrahydro-1H-pyrrolizin-7a(5H)-yl)methoxy)quinazolin-4-yl)piperazine-1-carboxylate (6.0 mg, 33%). LCMS(ESI+) m/z 804.00 [M+H]. ¹HNMR (300 MHz, CDCl₃) δ 7.50 (s, 1H), 7.37 (dd, J=8.4, 5.2 Hz, 1H), 7.10 (t, J=8.6 Hz, 1H), 6.35 (dd, J=76.5, 70.8 Hz, 1H), 4.70 (br s, 2H), 3.83 (br s, 4H), 3.68 (br s, 4H), 3.11-2.88 (m, 2H), 2.51-1.90 (m, 10H), 1.54 (s, 9H), 1.50 (s, 9H).

Step G: Preparation of 4-(6-(difluoromethoxy)-8-fluoro-4-(piperazin-1-yl)-2-((tetrahydro-1H-pyrrolizin-7a(5H)-yl)methoxy)quinazolin-7-yl)-7-fluorobenzo[d]thiazol-2-amine: To a stirring solution of tert-butyl 4-(7-(2-((tert-butoxycarbonyl)amino)-7-fluorobenzo[d]thiazol-4-yl)-6-(difluoromethoxy)-8-fluoro-2-((tetrahydro-1H-pyrrolizin-7a(5H)-yl)methoxy)quinazolin-4-yl)piperazine-1-carboxylate (5.7 mg, 0.007 mmol) in DCM (5 mL) at room temperature, TFA (0.27 mL, 3.55 mmol) was added and the reaction was allowed to stir at room temperature. After 2 hours of stirring at room temperature, the reaction mixture was concentrated in vacuo and co-distilled with DCM three times and dried under high vacuum. The product obtained was triturated with diethyl ether and dried overnight to afford 4-(6-(difluoromethoxy)-8-fluoro-4-(piperazin-1-yl)-2-((tetrahydro-1H-pyrrolizin-7a(5H)-yl)methoxy)quinazolin-7-yl)-7-fluorobenzo[d]thiazol-2-amine (3.8 mg, 57%) as TFA salt. LCMS (ESI+) m/z 604.14 [M+H]. ¹HNMR (300 MHz, CD₃OD) δ 7.58 (s, 1H), 7.27 (dd, J=8.5, 5.3 Hz, 1H), 7.00 (t, J=8.6 Hz, 1H), 6.83 (dd, J=76.4, 70.9 Hz, 1H), 4.67 (s, 2H), 4.11 (br s, 4H), 3.70 (dd, J=12.6, 6.1 Hz, 2H), 3.49 (br s, 4H), 3.19 (m, 2H, portion of these peaks were merged in CD₃OD residual peak), 2.40-1.99 (m, 8H).

### Synthetic Example 31: Synthesis of 7-fluoro-4-((S)-8-fluoro-4-(methyl((R)-pyrrolidin-3-yl)amino)-2-((tetrahydro-1H-pyrrolizin-7a(5H)-yl)methoxy)-6-(trifluoromethyl)quinazolin-7-yl)benzo[d]thiazol-2-amine (Compound 236) and 7-fluoro-4-((R)-8-fluoro-4-(methyl((R)-pyrrolidin-3-yl)amino)-2-((tetrahydro-1H-pyrrolizin-7a(5H)-yl)methoxy)-6-(trifluoromethyl)quinazolin-7-yl)benzo[d]thiazol-2-amine (Compound 238)

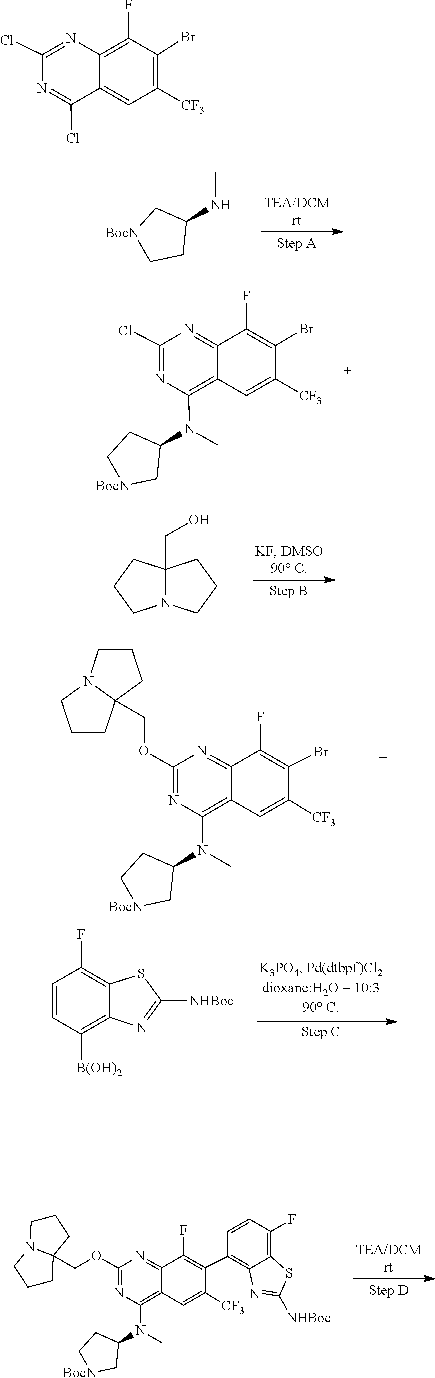

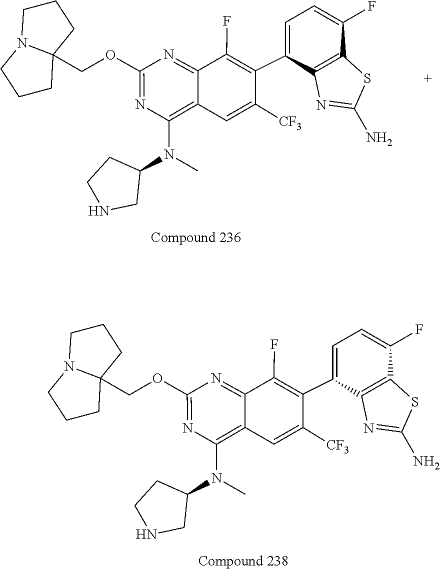

Step A: Preparation of tert-butyl (3R)-3-[[7-bromo-2-chloro-8-fluoro-6-(trifluoromethyl)quinazolin-4-yl]-methyl-amino]pyrrolidine-1-carboxylate: To a solution of 7-bromo-2,4-dichloro-8-fluoro-6-(trifluoromethyl)quinazoline (1.00 g, 2.75 mmol) in DCM (10 mL) were added TEA (1.1 mL, 8.24 mmol) and tert-butyl (3R)-3-(methylamino)pyrrolidine-1-carboxylate (605 mg, 3.02 mmol) at ambient temperature and stirred at this temperature for 2 hours. The reaction mixture was concentrated to dryness under vacuum. The crude product was purified by column chromatography on silica gel, eluting with 25% EtOAc in petroleum ether, to give tert-butyl (3R)-3-[[7-bromo-2-chloro-8-fluoro-6-(trifluoromethyl)quinazolin-4-yl]-methyl-amino]pyrrolidine-1-carboxylate (1.01 g, 69%).

Step B: Preparation of tert-butyl (3R)-3-[[7-bromo-8-fluoro-2-(1,2,3,5,6,7-hexahydropyrrolizin-8-ylmethoxy)-6-(trifluoromethyl)quinazolin-4-yl]-methyl-amino]pyrrolidine-1-carboxylate: To a solution of tert-butyl (3R)-3-[[7-bromo-2-chloro-8-fluoro-6-(trifluoromethyl)quinazolin-4-yl]-methyl-amino] pyrrolidine-1-carboxylate (150 mg, 0.28 mmol) in DMSO (5 mL) were added 1,2,3,5,6,7-hexahydropyrrolizin-8-ylmethanol (120 mg, 0.85 mmol) and KF (132 mg, 2.27 mmol) at ambient temperature. The mixture was stirred at 90° C. for 3 hours. After cooling to ambient temperature, ethyl acetate and water were added. The organic layers were separated, washed with brine solution, dried over Na₂SO₄, filtered, and concentrated under reduced pressure. The crude product was purified by preparative-TLC (DCM:MeOH=20:1 to give tert-butyl (3R)-3-[[7-bromo-8-fluoro-2-(1,2,3,5,6,7-hexahydropyrrolizin-8-ylmethoxy)-6-(trifluoromethyl)quinazolin-4-yl]-methyl-amino]pyrrolidine-1-carboxylate (104 mg, 57%). LCMS (ES+) m/z 632.2 (M+H).

Step C: Preparation of tert-butyl (3R)-3-[[7-[2-(tert-butoxycarbonylamino)-7-fluoro-1,3-benzothiazol-4-yl]-8-fluoro-2-(1,2,3,5,6,7-hexahydropyrrolizin-8-ylmethoxy)-6-(trifluoromethyl)quinazolin-4-yl]-methyl-amino]pyrrolidine-1-carboxylate: To a mixture of tert-butyl (3R)-3-[[7-bromo-8-fluoro-2-(1,2,3,5,6,7-hexahydropyrrolizin-8-ylmethoxy)-6-(trifluoromethyl)quinazolin-4-yl]-methyl-amino]pyrrolidine-1-carboxylate (104 mg, 0.16 mmol) in water (0.4 mL) and 1,4-dioxane (2 mL) were added [2-(tert-butoxycarbonylamino)-7-fluoro-1,3-benzothiazol-4-yl]boronic acid (56 mg, 0.18 mmol), 1,1′-bis (di-t-butylphosphino)ferrocene palladium dichloride (11 mg, 0.016 mmol) and potassium phosphate (70 mg, 0.33 mmol) at ambient temperature. The mixture was bubbled with argon for about 1-2 minutes and then sealed. After that, the mixture was stirred for 1 hour at 90° C. After cooling to ambient temperature, the reaction was concentrated to dryness and the residue was taken up in EtOAc (20 mL) and the organic layers washed with saturated brine solution. The organic layers were then separated, dried (MgSO₄), filtered and concentrated under reduced pressure. The crude was then purified by preparative-TLC (DCM/MeOH=10/1) to give product tert-butyl (3R)-3-[[7-[2-(tert-butoxycarbonylamino)-7-fluoro-1,3-benzothiazol-4-yl]-8-fluoro-2-(1,2,3,5,6,7-hexahydropyrrolizin-8-ylmethoxy)-6-(trifluoromethyl)quinazolin-4-yl]-methyl-amino]pyrrolidine-1-carboxylate (65 mg, 48%). LCMS (ES+) m/z 820.3 (M+H).

Step D: Preparation of 7-fluoro-4-((S)-8-fluoro-4-(methyl((R)-pyrrolidin-3-yl)amino)-2-((tetrahydro-1H-pyrrolizin-7a(5H)-yl)methoxy)-6-(trifluoromethyl)quinazolin-7-yl)benzo[d]thiazol-2-amine (Compound 236) and 7-fluoro-4-((R)-8-fluoro-4-(methyl((R)-pyrrolidin-3-yl)amino)-2-((tetrahydro-1H-pyrrolizin-7a(5H)-yl)methoxy)-6-(trifluoromethyl)quinazolin-7-yl)benzo[d]thiazol-2-amine (Compound 238): To a solution of tert-butyl (3R)-3-[[7-[2-(tert-butoxycarbonylamino)-7-fluoro-1,3-benzothiazol-4-yl]-8-fluoro-2-(1,2,3,5,6,7-hexahydropyrrolizin-8-ylmethoxy)-6-(trifluoromethyl)quinazolin-4-yl]-methyl-amino] pyrrolidine-1-carboxylate (20 mg, 0.024 mmol) in DCM (3 mL) was added TFA (1.5 mL, 19.5 mmol) at ambient temperature and the mixture was stirred at this temperature for 2 hours. Solvent was removed under vacuum and the residue was purified by preparative RP-HPLC. The first compound off the column was identified as one atropisomer, 7-fluoro-4-((S)-8-fluoro-4-(methyl((R)-pyrrolidin-3-yl)amino)-2-((tetrahydro-1H-pyrrolizin-7a(5H)-yl)methoxy)-6-(trifluoromethyl)quinazolin-7-yl)benzo[d]thiazol-2-amine (Compound 236) (3.0 mg, 19%). LCMS (ES+) m/z 620.3 (M+H). ¹HNMR (400 MHz, CD₃OD) δ 8.37 (s, 1H), 7.17-7.24 (m, 1H), 6.99 (t, J=8.9 Hz, 1H), 5.26-5.36 (m, 1H), 4.68 (s, 2H), 3.80-3.80 (m, 1H), 3.62-3.76 (m, 4H), 3.54 (s, 3H), 3.38-3.51 (m, 3H), 2.51-2.62 (m, 1H), 2.30-2.46 (m, 3H), 2.20-2.26 (m, 2H), 2.07-2.16 (m, 4H). The second compound off the column was identified as the other atropisomer, 7-fluoro-4-((R)-8-fluoro-4-(methyl((R)-pyrrolidin-3-yl)amino)-2-((tetrahydro-1H-pyrrolizin-7a(5H)-yl)methoxy)-6-(trifluoromethyl)quinazolin-7-yl)benzo[d]thiazol-2-amine (Compound 238) (3.6 mg, 23%). LCMS (ES+) m/z 620.3 (M+H). ¹HNMR (400 MHz, CD₃OD) δ 8.37 (s, 1H), 7.18-7.25 (m, 1H), 6.99 (t, J=8.8 Hz, 1H), 5.26-5.36 (m, 1H), 4.67 (s, 2H), 3.85 (dd, J=12.1, 8.9 Hz, 1H), 3.64-3.73 (m, 3H), 3.54 (s, 3H), 3.38-3.50 (m, 3H), 3.25-3.25 (m, 1H), 2.50-2.63 (m, 1H), 2.08-2.47 (m, 10H).

### Synthetic Example 32: Synthesis of 4-(4-((1R,5S)-3,8-diazabicyclo[3.2.1]octan-3-yl)-8-fluoro-2-((1-(morpholinomethyl)cyclopropyl)methoxy)-6-(trifluoromethyl)quinazolin-7-yl)-7-fluorobenzo[d]thiazol-2-amine (Compound 251)

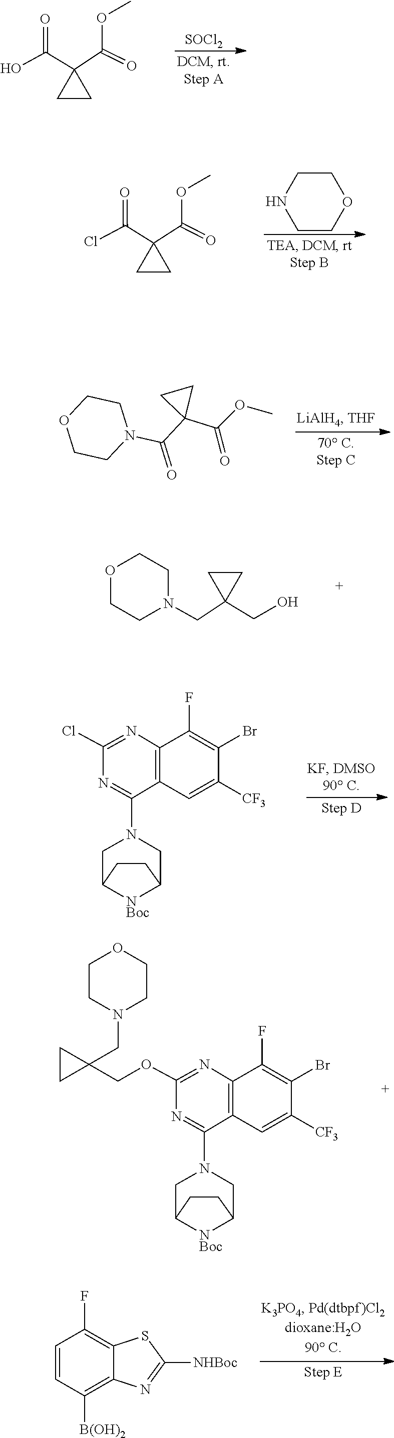

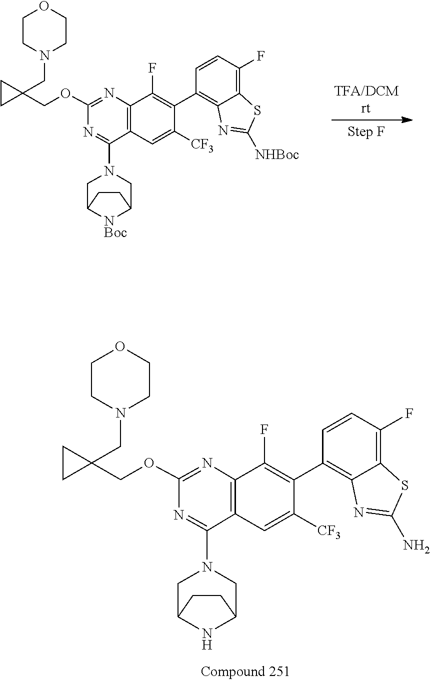 [SMILES: CC(C)(C)OC(=O)Nc1nc2c(-c3c(C(F)(F)F)cc4c(N5CC6CCC(C5)N6C(=O)OC(C)(C)C)nc(OCC5(CN6CCOCC6)CC5)nc4c3F)ccc(F)c2s1 ; Nc1nc2c(-c3c(C(F)(F)F)cc4c(N5CC6CCC(C5)N6)nc(OCC5(CN6CCOCC6)CC5)nc4c3F)ccc(F)c2s1]

Step A: Preparation of methyl 1-(chlorocarbonyl)cyclopropane-1-carboxylate: To a solution of 1-methoxycarbonylcyclopropanecarboxylic acid (200 mg, 1.39 mmol) in DCM (3 mL) and DMF (3 drops) was added SOCl₂ (539 mg, 3.47 mmol) at 0° C. The reaction was stirred at ambient temperature for 1.5 hours and the reaction was concentrated under reduced pressure to give methyl 1-(chlorocarbonyl) cyclopropane-1-carboxylate which was used directly for the next step.

Step B: Preparation of methyl 1-(morpholine-4-carbonyl)cyclopropane-1-carboxylate: To a mixture of morpholine (98 mg, 1.12 mmol) in DCM (3 mL) was added methyl 1-chlorocarbonyl cyclopropane carboxylate (182 mg, 1.12 mmol) in DCM (2 mL) and Et₃N (341 mg, 3.37 mmol) at 0° C. After addition, the mixture was warmed to ambient temperature and stirred at ambient temperature for 1 hour. Water was added. The organic layers were separated, washed with brine solution, dried over Na₂SO₄, filtered, and concentrated under reduced pressure to give methyl 1-(morpholine-4-carbonyl)cyclopropane-1-carboxylate (193 mg). LCMS (ES+) m/z 214.1 (M+H).

Step C: Preparation of (1-(morpholinomethyl)cyclopropyl)methanol: To a mixture of methyl 1-(morpholine-4-carbonyl)cyclopropanecarboxylate (193 mg, 0.91 mmol) in THE (6 mL) was added LiAlH₄ (85.9 mg, 2.26 mmol) at −20° C. After addition, the mixture was stirred at 70° C. for 5 hours. After cooled to ambient temperature, the reaction was slowly added to water, NaOH (15%) and water. The solid was removed by filtration, and washed with ethyl acetate. The organic layers were separated, washed with brine solution, dried over Na₂SO₄, filtered, and concentrated under reduced pressure to give [1-(morpholinomethyl)cyclopropyl]methanol (106 mg, 0.62 mmol). LCMS (ES+) m/z 172.13 (M+H).

Step D: Preparation of tert-butyl (1R,5S)-3-(7-bromo-8-fluoro-2-((1-(morpholinomethyl)cyclopropyl)methoxy)-6-(trifluoromethyl)quinazolin-4-yl)-3,8-diazabicyclo[3.2.1]octane-8-carboxylate: A mixture of [1-(morpholinomethyl)cyclopropyl]methanol (38 mg, 0.22 mmol), tert-butyl (1R,5S)-3-[7-bromo-2-chloro-8-fluoro-6-(trifluoromethyl)quinazolin-4-yl]-3,8-diazabicyclo[3.2.1]octane-8-carboxylate (100 mg, 0.19 mmol), and KF (86 mg, 1.48 mmol) in DMSO was stirred at 90° C. under Ar for 3 hours. After cooled to ambient temperature, ethyl acetate and water were added. The organic layers were separated, washed with brine solution, dried over Na₂SO₄, filtered, and concentrated under reduced pressure. The crude product was purified by preparative-TLC (100% EtOAc) to give tert-butyl (1R,5S)-3-[7-bromo-8-fluoro-2-[[1-(morpholinomethyl)cyclopropyl]methoxy]-6-(trifluoromethyl)quinazolin-4-yl]-3,8-diazabicyclo[3.2.1]octane-8-carboxylate (55 mg, 43%). LCMS (ES+) m/z 674.19 (M+H).

Step E: Preparation of tert-butyl (1R,5S)-3-(7-(2-((tert-butoxycarbonyl)amino)-7-fluorobenzo[d]thiazol-4-yl)-8-fluoro-2-((1-(morpholinomethyl)cyclopropyl)methoxy)-6-(trifluoromethyl)quinazolin-4-yl)-3,8-diazabicyclo[3.2.1]octane-8-carboxylate: A mixture of tert-butyl (1R,5S)-3-[7-bromo-8-fluoro-2-[[1-(morpholinomethyl)cyclopropyl]methoxy]-6-(trifluoromethyl)quinazolin-4-yl]-3,8-diazabicyclo[3.2.1]octane-8-carboxylate (55 mg, 0.081 mmol), [2-(tert-butoxycarbonylamino)-7-fluoro-1,3-benzothiazol-4-yl]boronic acid (25 mg, 0.081 mmol), Pd(dtbpf)Cl₂ (4.2 mg, 0.0065 mmol), and K₃PO₄ (69 mg, 0.33 mmol) in 1,4-dioxane (1 mL)/water (0.30 mL) was stirred at 90° C. under Ar for 2 hours. After cooled to ambient temperature, the reaction was concentrated to dryness and the residue was taken up in EtOAc (20 mL) and the organic layers washed with saturated brine solution. The organic layers were then separated, dried (MgSO₄), filtered and concentrated under reduced pressure. The crude was then purified by preparative-TLC (1:1-hexane/EtOAc) to give tert-butyl (1R,5S)-3-[7-[2-(tert-butoxycarbonylamino)-7-fluoro-1,3-benzothiazol-4-yl]-8-fluoro-2-[[1-(morpholinomethyl)cyclopropyl]methoxy]-6-(trifluoromethyl)quinazolin-4-yl]-3,8-diazabicyclo[3.2.1]octane-8-carboxylate (30 mg, 43%). LCMS (ES+) m/z 862.33 (M+H).

Step F: Preparation of 4-(4-((1R,5S)-3,8-diazabicyclo[3.2.1]octan-3-yl)-8-fluoro-2-((1-(morpholinomethyl)cyclopropyl)methoxy)-6-(trifluoromethyl)quinazolin-7-yl)-7-fluorobenzo[d]thiazol-2-amine: To a solution of tert-butyl (1R,5S)-3-[7-[2-(tert-butoxycarbonylamino)-7-fluoro-1,3-benzothiazol-4-yl]-8-fluoro-2-[[1-(morpholinomethyl)cyclopropyl]methoxy]-6-(trifluoromethyl)quinazolin-4-yl]-3,8-diazabicyclo[3.2.1]octane-8-carboxylate (30 mg, 0.035 mmol) in DCM (1 mL) was added TFA (0.50 mL) at ambient temperature. The mixture was stirred at ambient temperature for 2 hours. The reaction was concentrated and purified by preparative RP-HPLC to give 4-[4-[(1R,5S)-3,8-diazabicyclo[3.2.1]octan-3-yl]-8-fluoro-2-[[1-(morpholinomethyl)cyclopropyl]methoxy]-6-(trifluoromethyl)quinazolin-7-yl]-7-fluoro-1,3-benzothiazol-2-amine (14 mg, 58%). LCMS (ES+) m/z 662.23 (M+H). ¹HNMR (400 MHz, CD₃OD) δ 8.14 (s, 1H), 7.17-7.22 (m, 1H), 7.0 (t, J=8.72 Hz, 1H), 4.69 (t, J=11.84 Hz, 2H), 4.48 (q, J=11.96 Hz, 49.08 Hz, 4H), 4.25 (s, 2H), 4.00-4.12 (m, 2H), 3.70-3.96 (m, 6H), 3.36 (d, J=13.64 Hz, 26.76 Hz, 2H), 3.12-3.26 (m, 2H), 2.07-2.19 (m, 4H), 0.97-1.02 (m, 2H), 0.85-0.92 (m, 2H).

### Synthetic Example 33: Synthesis of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 255), 4-((S)-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 366) and 4-((R)-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 367)

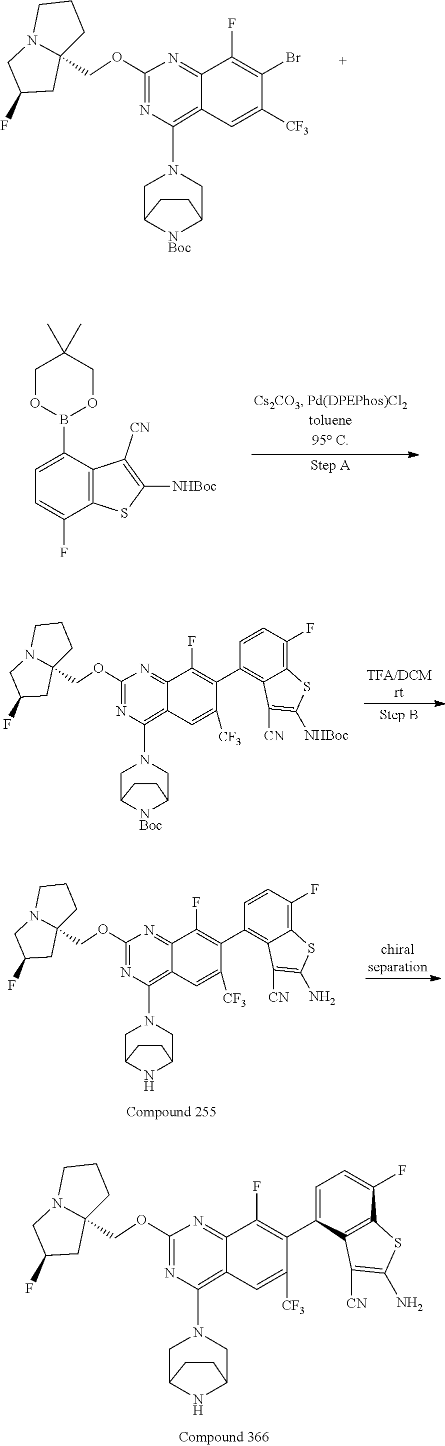

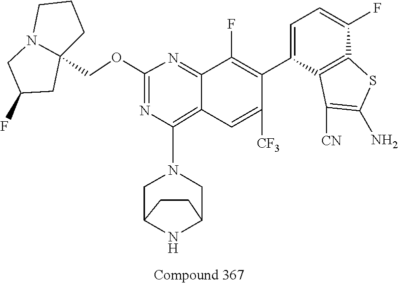

Step A: Preparation of tert-butyl (1R,5S)-3-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate: To a solution of tert-butyl 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (50 mg, 0.076 mmol) in toluene (2 mL) were added tert-butyl N-[3-cyano-4-(5,5-dimethyl-1,3,2-dioxaborinan-2-yl)-7-fluoro-benzothiophen-2-yl]carbamate (46 mg, 0.11 mmol), cesium carbonate (61 mg, 0.19 mmol) and DPEPhosPdCl₂ (8.1 mg, 0.011 mmol) at room temperature under nitrogen. The mixture was stirred at 95° C. for 1 hour. After cooled to ambient temperature, purified water was added, extracted with ethyl acetate for three times, the organic phase was washed with saturated sodium chloride solution, dried with anhydrous sodium sulfate, filtered and concentrated under reduced pressure. The residue obtained was purified by preparative-TLC (DCM/MeOH=20/1) to give tert-butyl 3-[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (48 mg, 72%).

Step B: Preparation of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 255), 4-((S)-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 366) and 4-((R)-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 367): To a solution of tert-butyl 3-[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (58 mg, 0.066 mmol) in DCM (3 mL) was added trifluoroacetic acid (1.5 mL, 19.5 mmol) at room temperature, The mixture was stirred at room temperature for 2 hours. The mixture was concentrated to dryness under vacuum and the residue was purified by Preparative RP-HPLC to afford 2-amino-4-[4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (22 mg, 417%). LCMS ESI (+) m/z 674.21 (M+H). ¹HNMR (400 MHz, CD₃OD) δ 8.17 (s, 1H), 7.22 (dd, J=8.4, 5.2 Hz, 1H), 7.03 (t, J=8.8 Hz, 1H), 5.57 (d, J=52 Hz, 1H), 4.64-4.83 (m, 4H), 4.25 (s, 2H), 3.84-4.08 (m, 5H), 3.43-3.53 (m, 1H), 2.58-2.80 (m, 2H), 2.30-2.50 (m, 3H), 2.03-2.24 (m, 5H). The enantiomers of 4-[4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-1,3-benzothiazol-2-amine (Compound 255) (50.6 mg) were separated with chiral chromatography condition {ChiralPak IH 3 cm×25 cm, 5 μm, CO2:[IPA:DCM=2:1 (0.2% 2 mM ammonium in methanol)], 80 mL/min}. The first compound off the column was identified as one atropisomer, 4-((S)-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 366) (11.6 mg, 99.8% de); LCMS ESI (+) m/z 674.3 (M+H). ¹HNMR (300 MHz, CD₃OD) δ 8.13 (s, 1H), 7.22 (dd, J=8.4, 5.4 Hz, 1H), 7.02 (t, J=9.0 Hz, 1H), 5.36 (d, J=53.7 Hz, 1H), 4.57 (d, J=12.6 Hz, 1H), 4.52 (d, J=11.7 Hz, 1H), 4.32 (d, J=10.5 Hz, 1H), 4.22 (d, J=10.5 Hz, 1H), 3.59-3.81 (m, 4H), 3.19-3.30 (m, 2H), 2.91-3.15 (m, 1H), 2.99-3.12 (m, 1H), 2.11-2.42 (m, 3H), 1.93-2.10 (m, 2H), 1.78-1.92 (m, 5H). The second compound off the column was identified as the other atropisomer, 4-((R)-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 367) (13.7 mg, 99.4% de). LCMS ESI (+) m/z 674.3 (M+H). ¹HNMR (300 MHz, CD₃OD) δ 8.13 (s, 1H), 7.22 (dd, J=8.4, 5.7 Hz, 1H), 7.02 (t, J=9.0 Hz, 1H), 5.33 (d, J=53.4 Hz, 1H), 4.60 (d, J=13.5 Hz, 1H), 4.47 (d, J=12.0 Hz, 1H), 4.33 (d, J=10.5 Hz, 1H), 4.25 (d, J=10.8 Hz, 1H), 3.59-3.81 (m, 4H), 3.21-3.45 (m, 3H), 2.95-3.14 (m, 1H), 2.12-2.48 (m, 3H), 1.97-2.10 (m, 2H), 1.78-1.96 (m, 5H).

### Synthetic Example 34: Synthesis of 4-(4-((2S,5R)-2,5-dimethylpiperazin-1-yl)-8-fluoro-2-(((2R,7aS)-2-fluorotetrahydro-1H-pyrrolizin-7a(5H)-yl)methoxy)-6-methoxyquinazolin-7-yl)-7-fluorobenzo[d]thiazol-2-amine (Compound 281)

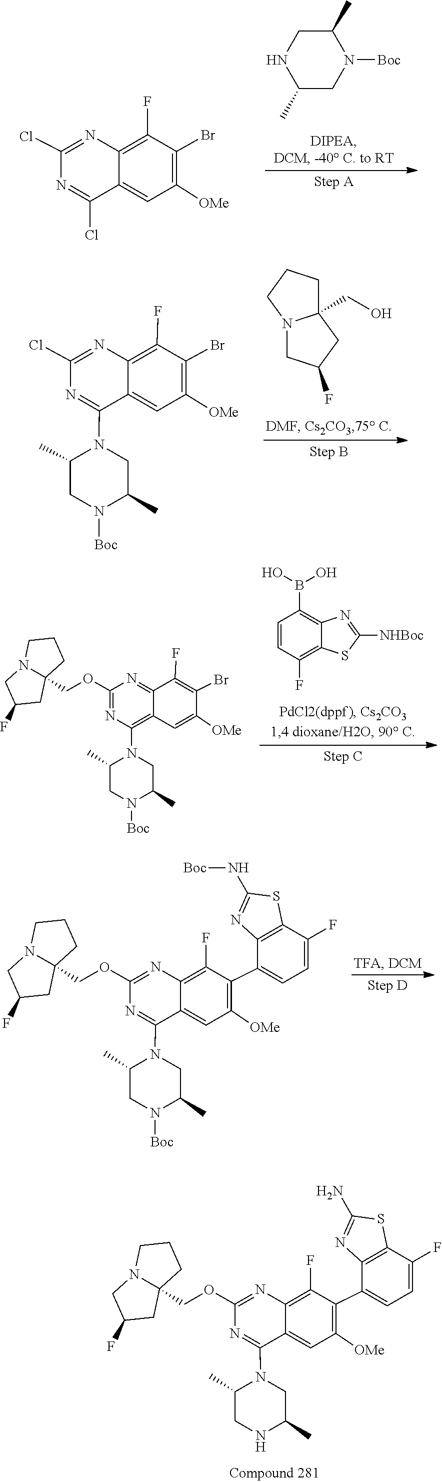

Step A: Preparation of tert-butyl (2R,5S)-4-(7-bromo-2-chloro-8-fluoro-6-methoxyquinazolin-4-yl)-2,5-dimethylpiperazine-1-carboxylate: A solution of 7-bromo-2,4-dichloro-8-fluoro-6-methoxy-quinazoline (460 mg, 1.41 mmol) in DCM (4.6 mL) was stirred at −40° C. under argon. To this, DIPEA (0.74 mL, 4.23 mmol) and tert-butyl (2R,5S)-2,5-dimethylpiperazine-1-carboxylate (363 mg, 1.69 mmol) were added subsequently, and the reaction mixture was gradually allowed to warm to room temperature. After 3 hours of stirring, the reaction mixture was concentrated under vacuum and the resultant crude was purified by flash chromatography on silica gel (eluted with 30-35% EtOAc/hexane) provided desired product (248 mg, 35% yield) as a white solid. ¹H NMR (300 MHz, CDCl₃) δ 6.89 (s, 1H), 4.76-4.64 (m, 1H), 4.25-4.56 (m, 1H), 4.06-3.97 (m, 4H; it is mixture of doublet at 4.03 ppm and singlet at 3.99 ppm), 3.78 (dd, J=13.6, 4.2 Hz, 2H), 3.47 (br s, 1H), 1.50 (s, 9H), 1.41 (d, J=6.6 Hz, 3H), 1.22 (d, J=6.6 Hz, 3H).

Step B: Preparation of tert-butyl (2R,5S)-4-(7-bromo-8-fluoro-2-(((2R,7aS)-2-fluorotetrahydro-1H-pyrrolizin-7a(5H)-yl)methoxy)-6-methoxyquinazolin-4-yl)-2,5-dimethylpiperazine-1-carboxylate: In a sealed tube, to a stirring solution of tert-butyl (2R,5S)-4-(7-bromo-2-chloro-8-fluoro-6-methoxyquinazolin-4-yl)-2,5-dimethylpiperazine-1-carboxylate (248 mg, 0.49 mmol) in DMF (3.2 mL), ((2R,7aS)-2-fluorotetrahydro-1H-pyrrolizin-7a(5H)-yl)methanol (157 mg, 0.99 mmol) and Cs₂CO₃ (321 mg, 0.985 mmol) were added at room temperature and the reaction mixture was heated at 75° C. After 36 hours of heating, the reaction mixture was cooled to room temperature and water/EtOAc were added. Layers were separated and the aqueous layer was further extracted with EtOAc (2×20 mL). The organic layers were combined, dried over Na₂SO₄, filtered, concentrated in vacuo. The crude residue was purified by flash chromatography on silica gel (eluted in 5-7% MeOH in DCM) to afford title compound (64 mg, 21% yield) as a white solid. LRMS (ESI+) m/z [M+H] 626.44 and [M+2+H] 628.40. ¹H NMR (300 MHz, CDCl₃) δ 6.87 (s, 1H), 5.40-5.15 (m, 1H), 4.66 (br s, 1H), 4.42 (br s, 1H), 4.18 (q, J=10.3 Hz, 2H), 3.95 (m, 4H), 3.84-3.68 (m, 2H), 3.53-3.49 (m, 1H), 3.33-3.23 (m, 2H), 3.15 (s, 1H), 3.03-2.90 (m, 1H), 2.29 (s, 1H), 2.21-2.11 (m, 2H), 1.98-1.90 (m, 3H), 1.49 (s, 9H), 1.34 (d, J=6.6 Hz, 3H), 1.21 (d, J=6.8 Hz, 3H).

Step C: Preparation of tert-butyl (2R,5S)-4-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-methoxyquinazolin-4-yl)-2,5-dimethylpiperazine-1-carboxylate: To a stirring suspension of tert-butyl (2R,5S)-4-(7-bromo-8-fluoro-2-(((2R,7aS)-2-fluorotetrahydro-1H-pyrrolizin-7a(5H)-yl)methoxy)-6-methoxyquinazolin-4-yl)-2,5-dimethylpiperazine-1-carboxylate (63 mg, 0.101 mmol) in 1,4-dioxane (1.5 mL), (2-((tert-butoxycarbonyl)amino)-7-fluorobenzo[d]thiazol-4-yl)boronic acid (63 mg, 0.201 mmol) and an aqueous solution of Cs₂CO₃ (66 mg, 0.201 mmol) in water (0.15 mL) were added. The reaction mixture was degassed with argon for 10 minutes, then Pd(dppf)Cl₂·DCM (12 mg, 0.0151 mmol) was added and degassed again for 10 min. The reaction mixture was heated to 90° C. for 90 minutes. The reaction mixture was cooled to room temperature and diluted with water/EtOAc. Layers were separated and the aqueous layer was further extracted with EtOAc (2×15 mL). The organic layers were combined, washed with brine, dried over Na₂SO₄, filtered, and concentrated in vacuo. The resultant crude was purified by flash chromatography on silica gel (eluted with 60-68% EtOAc in hexane) to afford title compound (50 mg, 61% yield). LRMS (ESI+) m/z 814.58 [M+H]. ¹H NMR (300 MHz, CDCl₃) δ 7.32 (td, J=8.7, 5.4 Hz, 1H), 7.08 (td, J=8.5, 2.1 Hz, 1H), 6.94 (d, J=10.2 Hz, 1H), 5.23 (d, J=53.9 Hz, 1H), 4.67 (br s, 1H), 4.42-4.58 (br s, 1H), 4.25-4.08 (m, 2H), 3.94 (dd, J=31.3, 13.7 Hz, 1H), 3.84-3.68 (m, 4H), 3.51 (t, J=13.7 Hz, 1H), 3.36-3.09 (m, 3H), 2.98-2.85 (m, 1H), 2.82-2.08 (m, 3H), 1.98-1.74 (m, 4H), 1.53 (d, J=9.4 Hz, 9H), 1.50 (d, J=9.4 Hz, 9H), 1.37-1.21 (m, 6H).

Step D: Preparation of 4-(4-((2S,5R)-2,5-dimethylpiperazin-1-yl)-8-fluoro-2-(((2R,7aS)-2-fluorotetrahydro-1H-pyrrolizin-7a(5H)-yl)methoxy)-6-methoxyquinazolin-7-yl)-7-fluorobenzo[d]thiazol-2-amine: To a stirring solution of tert-butyl (2R,5S)-4-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-methoxyquinazolin-4-yl)-2,5-dimethylpiperazine-1-carboxylate (51 mg, 0.0627 mmol) in DCM (5 mL) at room temperature, TFA (1 mL) was added. After 3 hours of stirring at room temperature, the reaction mixture was concentrated in vacuo and co-distilled with DCM three times. The product obtained was triturated with diethyl ether twice, decanted, and dried under high vacuum to afford title compound as TFA salt (57 mg, 95% yield). LCMS (ESI+) m/z 614.35 [M+H]. ¹H NMR (300 MHz, CD₃OD) δ 7.33-7.18 (m, 2H), 7.06 (t, J=9.2 Hz, 1H), 5.58 (d, J=51.8 Hz, 1H), 4.69 (s, 2H), 4.38 (br s, 1H), 4.13-3.73 (m, 8H), 3.70-3.35 (m, 4H), 2.82-2.56 (m, 2H), 2.48-2.26 (m, 3H), 2.21-2.08 (m, 1H), 1.46 (dd, J=6.5, 2.1 Hz, 3H), 1.41 (dd, J=6.7, 2.4 Hz, 3H).

### Synthetic Example 35: 4-(6-(difluoromethoxy)-4-((2S,5R)-2,5-dimethylpiperazin-1-yl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine (Compound 282)

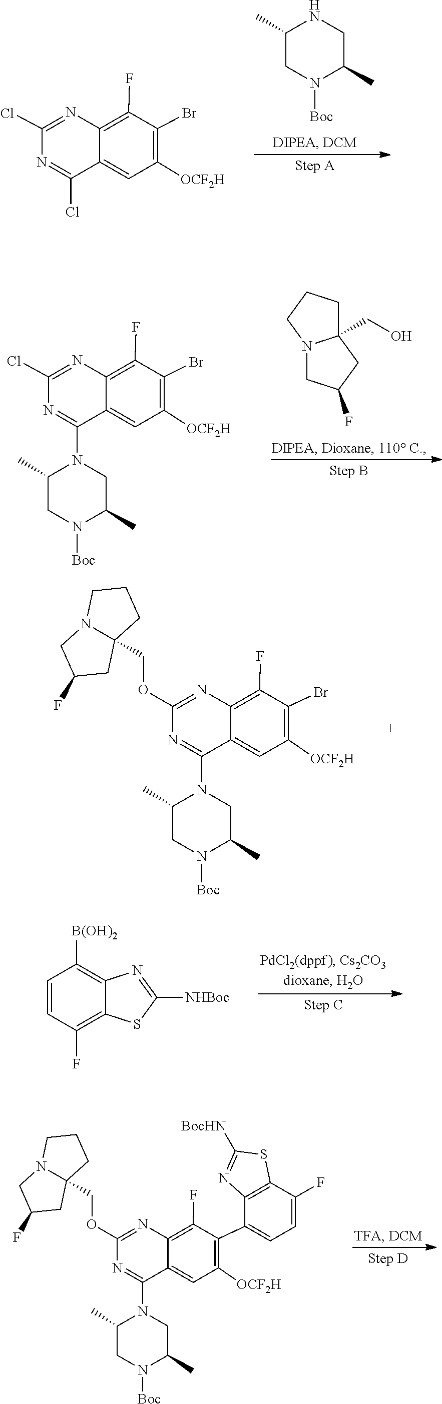

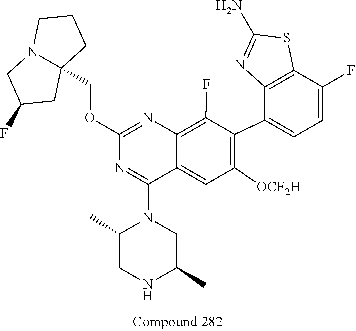

Step A: Preparation of tert-butyl (2R,5S)-4-(7-bromo-2-chloro-6-(difluoromethoxy)-8-fluoroquinazolin-4-yl)-2,5-dimethylpiperazine-1-carboxylate: A solution of 7-bromo-2,4-dichloro-6-(difluoromethoxy)-8-fluoroquinazoline (0.24 g, 0.7 mmol) in DCM (5 mL) was cooled at −40° C. under argon. To this tert-butyl (2R,5S)-2,5-dimethylpiperazine-1-carboxylate (171 mg, 0.80 mmol) and DIPEA (0.35 mL, 2.0 mmol) were added subsequently. Then the reaction was continued in a same cooling bath for 15 minutes without any external heat. The reaction mixture was diluted with DCM (10 mL) and washed with water (1×20 mL), dried over Na₂SO₄, concentrated under vacuum. The crude was purified by flash column chromatography on silica gel using 0-45% EtOAc in hexane as eluent to give tert-butyl (2R,5S)-4-(7-bromo-2-chloro-6-(difluoromethoxy)-8-fluoroquinazolin-4-yl)-2,5-dimethylpiperazine-1-carboxylate (223 mg, 62%) as a white solid. ¹H NMR (300 MHz, CDCl₃) δ 7.54 (s, 1H), 6.78 (d, J=72.2 Hz, 1H), 4.71 (s, 1H), 4.57-4.37 (brs, 1H), 4.17-4.08 (m, 1H), 3.84-3.73 (m, 2H), 3.30-3.50 (m, 1H), 1.52 (s, 9H), 1.46 (d, J=6.7 Hz, 3H), 1.24 (d, J=6.9 Hz, 3H).

Step B: Preparation of tert-butyl (2R,5S)-4-(7-bromo-6-(difluoromethoxy)-8-fluoro-2-(((2R,7aS)-2-fluorotetrahydro-1H-pyrrolizin-7a(5H)-yl)methoxy)quinazolin-4-yl)-2,5-dimethylpiperazine-1-carboxylate: To a stirring suspension of (2R,5S)-4-[7-bromo-2-chloro-6-(difluoromethoxy)-8-fluoro-quinazolin-4-yl]-2,5-dimethyl-piperazine-1-carboxylate (25 mg, 0.0463 mmol) in dioxane, [(2R)-2-fluoro-1,2,3,5,6,7-hexahydropyrrolizin-8-yl]methanol (15 mg, 0.093 mmol) and DIPEA (0.0081 mL, 0.046 mmol) were added subsequently. The reaction mixture was heated to 110° C. for 70 h. After 70 h of heating, the reaction mixture was concentrated in vacuo to remove the solvent and water/EtOAc were added. The aqueous layer was further extracted with EtOAc (2×30 mL) and water (1×20 mL). Combined the organic layers, dried over Na₂SO₄, filtered, concentrated in vacuo. The resultant crude was purified by flash chromatography on silica gel using 20-30% of DCM in methanol as eluent to give tert-butyl (2R,5S)-4-(7-bromo-6-(difluoromethoxy)-8-fluoro-2-(((2R,7aS)-2-fluorotetrahydro-1H-pyrrolizin-7a(5H)-yl)methoxy)quinazolin-4-yl)-2,5-dimethylpiperazine-1-carboxylate (10 mg, 33%). LCMS (ESI+): 662.29 [M+H]. ¹H NMR (300 MHz, CDCl₃) δ 7.46 (s, 1H), 6.58 (t, J=72.8 Hz, 1H), 5.27 (d, J=55.57 Hz, 1H), 4.65 (s, 1H), 4.50 (brs, 1H) 4.21 (t, J=8.6 Hz, 2H), 3.96 (d, J=13.5 Hz, 1H), 3.81-3.67 (m, 2H), 3.49 (s, 1H), 3.25 (s, 2H), 3.16 (s, 1H), 3.02-2.92 (m, 1H), 2.29 (s, 1H), 2.21-2.10 (m, 2H), 1.98-1.85 (m, 3H), 1.49 (s, 9H), 1.36 (d, J=6.6 Hz, 3H), 1.21 (d, J=6.8 Hz, 3H).

Step C: Preparation of tert-butyl (2R,5S)-4-(7-(2-((tert-butoxycarbonyl)amino)-7-fluorobenzo[d]thiazol-4-yl)-6-(difluoromethoxy)-8-fluoro-2-(((2R,7aS)-2-fluorotetrahydro-1H-pyrrolizin-7a(5H)-yl)methoxy)quinazolin-4-yl)-2,5-dimethylpiperazine-1-carboxylate: A mixture of tert-butyl (2R,5S)-4-(7-bromo-6-(difluoromethoxy)-8-fluoro-2-(((2R,7aS)-2-fluorotetrahydro-1H-pyrrolizin-7a(5H)-yl)methoxy)quinazolin-4-yl)-2,5-dimethylpiperazine-1-carboxylate (80 mg, 0.12 mmol) and [2-(tert-butoxycarbonylamino)-7-fluoro-1,3-benzothiazol-4-yl]boronic acid (75 mg, 0.24 mmol) in 1,4-dioxane (1.5 mL) and water (0.15 mL) was degassed with argon, followed by the addition of 1,1′-bis(diphenylphosphino)ferrocenedichloropalladium(II) dichloromethane complex (15 mg, 0.018 mmol). The mixture was degassed with argon and heated at 90° C. for 2 h. After cooled to ambient temperature, the resulting mixture was concentrated under reduced pressure to give the crude residue which was purified by silica gel column chromatography using 0-30% MeOH in DCM as eluent to give tert-butyl (2R,5S)-4-(7-(2-((tert-butoxycarbonyl)amino)-7-fluorobenzo[d]thiazol-4-yl)-6-(difluoromethoxy)-8-fluoro-2-(((2R,7aS)-2-fluorotetrahydro-1H-pyrrolizin-7a(5H)-yl)methoxy)quinazolin-4-yl)-2,5-dimethylpiperazine-1-carboxylate (58 mg, 56%) as a brown gum. LCMS (ESI+): 850.54 [M+H]. ¹H NMR (301 MHz, CDCl₃) δ 7.49 (d, J=12.2 Hz, 1H), 7.37 (ddd, J=10.3, 8.4, 5.2 Hz, 1H), 7.12 (td, J=8.6, 1.8 Hz, 1H), 6.61-6.08 (m, 1H), 5.27 (d, J=53.8 Hz, 1H), 4.70-4.50 (m, 1H), 5.18 (s, 1H), 4.69 (s, 2H), 4.26-3.92 (m, 4H), 3.75 (t, J=13.3 Hz, 2H), 3.33-3.23 (m, 1H), 3.18 (d, J=9.5 Hz, 1H), 3.02-2.92 (s, 1H), 2.28-2.25 (m, 1H), 2.20-2.09 (m, 2H), 1.96-1.87 (m, 3H), 1.60-1.49 (m, 18H), 1.38 (dd, J=11.1, 6.5 Hz, 3H), 1.30-1.22 (m, 3H).

Step D: Preparation of 4-(6-(difluoromethoxy)-4-((2S,5R)-2,5-dimethylpiperazin-1-yl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine: To a stirring solution of tert-butyl (2R,5S)-4-(7-(2-((tert-butoxycarbonyl)amino)-7-fluorobenzo[d]thiazol-4-yl)-6-(difluoromethoxy)-8-fluoro-2-(((2R,7aS)-2-fluorotetrahydro-1H-pyrrolizin-7a(5H)-yl)methoxy)quinazolin-4-yl)-2,5-dimethylpiperazine-1-carboxylate (5.2 mg, 0.0061 mmol) in DCM (1 mL) at room temperature, TFA (2 mL, 9.09 mmol) was added and the mixture was stirred at room temperature for 2 h. After 2 h of stirring at room temperature, the reaction mixture was concentrated in vacuo and co-distilled with DCM three times and dried under high vacuum. The product obtained was triturated with diethyl ether and dried overnight to give 4-(6-(difluoromethoxy)-4-((2S,5R)-2,5-dimethylpiperazin-1-yl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine (6.9 mg, 97%) as a TFA salt. LCMS (ESI+): 650.4. ¹H NMR (300 MHz, CD₃OD) δ 7.64 (s, 1H), 7.28 (dd, J=8.4, 5.4 Hz, 1H), 7.03 (d, J=8.8 Hz, 1H), 7.14-6.60 (m, 1H), 5.58 (d, J=51.3 Hz, 1H), 4.70 (s, 2H), 4.46 (s, 1H), 4.04-3.68 (m, 8H), 3.48 (d, J=7.0 Hz, 1H), 2.80-2.57 (m, 2H), 2.38 (dt, J=17.6, 8.6 Hz, 3H), 2.19 (s, 1H), 1.51 (dd, J=6.6, 3.9 Hz, 3H), 1.45-1.38 (m, 3H).

### Synthetic Example 36: 4-(6-(1,1-difluoroethyl)-8-fluoro-4-(piperazin-1-yl)-2-((tetrahydro-1H-pyrrolizin-7a(5H)-yl)methoxy)quinazolin-7-yl)-7-fluorobenzo[d]thiazol-2-amine (Compound 295)

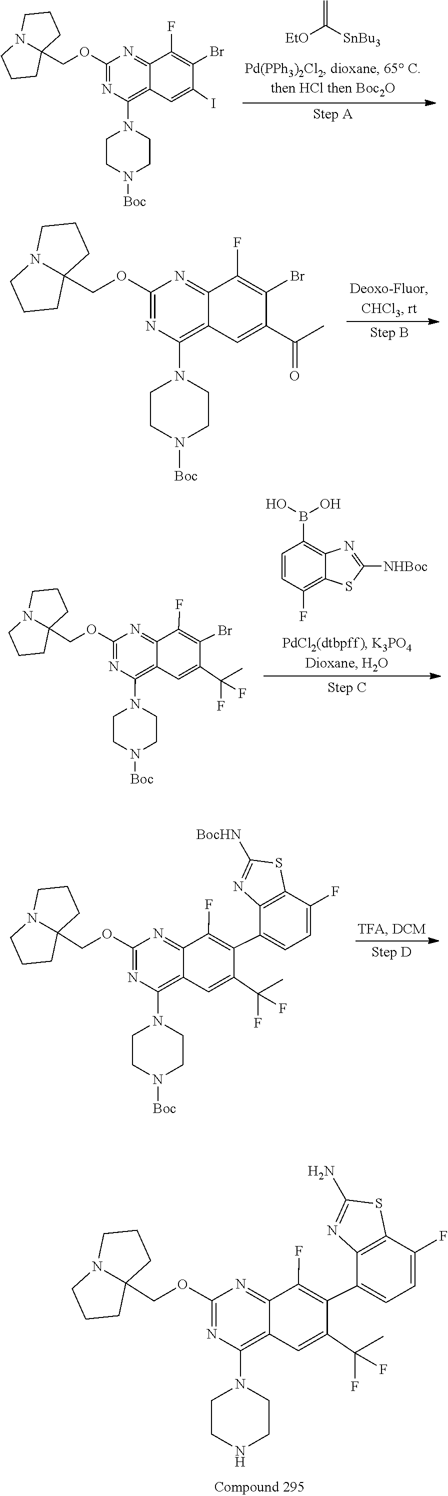

Step A: Preparation of tert-butyl 4-(6-acetyl-7-bromo-8-fluoro-2-((tetrahydro-1H-pyrrolizin-7a(5H)-yl)methoxy)quinazolin-4-yl)piperazine-1-carboxylate: A solution of 1-ethoxyvinyltri-n-butyltin (0.40 mL, 1.18 mmol), tert-butyl-4-(7-bromo-8-fluoro-6-iodo-2-((tetrahydro-1H-pyrrolizin-7a(5H)-yl)methoxy)quinazolin-4-yl)piperazine-1-carboxylate (0.40 g, 0.59 mmol), bis(triphenylphosphine)palladium chloride (42 mg, 0.060 mmol) in 1,4-dioxane (20 mL) was stirred at 65° C. for 5 h (product confirmed by MS). After completion, the solution was added HCl (9 mL) was stirred at 25° C. for 3 h. Then the solvent was concentrated under vacuum. The residue was dissolved with water and the pH of the resulting solution was adjusted to pH=9 with potassium carbonate. Di-tert-butyl-dicarbonate was added and the solution was stirred at 25° C. for 2 h. After completion of the reaction, the solution was extracted with EtOAc, and the organic layer was dried over sodium sulphate and concentrated under vacuum. The residue obtained was purified by column chromatography using 0-10% MeOH in DCM as eluent to give tert-butyl 4-(6-acetyl-7-bromo-8-fluoro-2-((tetrahydro-1H-pyrrolizin-7a(5H)-yl)methoxy)quinazolin-4-yl)piperazine-1-carboxylate (0.16 g, 46%) as a yellow solid. LCMS (ESI+): 592.24 [M+H]. ¹H NMR (300 MHz, CDCl₃) δ 7.81 (d, J=1.8 Hz, 1H), 4.27 (s, 2H), 3.83 (t, J=5.0 Hz, 4H), 3.67-3.58 (m, 4H), 3.21 (dt, J=11.1, 5.8 Hz, 2H), 2.73-2.60 (m, 5H), 2.10 (dt, J=12.4, 6.1 Hz, 2H), 1.95-1.84 (m, 4H), 1.74-1.63 (m, 2H), 1.47 (s, 9H).

Step B: Preparation of tert-butyl 4-(7-bromo-6-(1,1-difluoroethyl)-8-fluoro-2-((tetrahydro-1H-pyrrolizin-7a(5H)-yl)methoxy)quinazolin-4-yl)piperazine-1-carboxylate: To a solution of tert-butyl 4-(6-acetyl-7-bromo-8-fluoro-2-((tetrahydro-1H-pyrrolizin-7a(5H)-yl)methoxy)quinazolin-4-yl)piperazine-1-carboxylate (0.16 g, 0.26 mmol) in chloroform (2 mL) was added [bis(2-methoxyethyl)amino]sulfur trifluoride (0.48 mL, 2.63 mmol) at 0° C. The mixture was stirred at rt for 48 h. The reaction mixture was quenched with water (2 mL) and extracted with EtOAc and water, the EtOAc was collected, dried over sodium sulfate, filtered, and evaporated. The mixture was purified on a 4 g column using 0 to 10% MeOH in DCM as eluent to give tert-butyl 4-(7-bromo-6-(1,1-difluoroethyl)-8-fluoro-2-((tetrahydro-1H-pyrrolizin-7a(5H)-yl)methoxy)quinazolin-4-yl)piperazine-1-carboxylate (55 mg, 34%) as a colorless gum. LCMS (ESI+): 614.28 [M+H]. ¹H NMR (301 MHz, CD₃OD) δ 8.13-7.95 (m, 1H), 4.64 (s, 2H), 4.06-3.96 (m, 4H), 3.77-3.60 (m, 6H), 3.41-3.34 (m, 1H), 3.28 (d, J=6.2 Hz, 1H), 2.41-2.06 (m, 11H), 1.51 (s, 9H).

Step C: Preparation of tert-butyl 4-(7-(2-((tert-butoxycarbonyl)amino)-7-fluorobenzo[d]thiazol-4-yl)-6-(1,1-difluoroethyl)-8-fluoro-2-((tetrahydro-1H-pyrrolizin-7a(5H)-yl)methoxy)quinazolin-4-yl)piperazine-1-carboxylate: To a mixture of tert-butyl 4-(7-bromo-6-(1,1-difluoroethyl)-8-fluoro-2-((tetrahydro-1H-pyrrolizin-7a(5H)-yl)methoxy)quinazolin-4-yl)piperazine-1-carboxylate (55 mg, 0.090 mmol) in 1,4-dioxane (2 mL) and water (0.3 mL) was added [2-(tert-butoxycarbonylamino)-7-fluoro-1,3-benzothiazol-4-yl]boronic acid (56 mg, 0.18 mmol). The mixture was degassed with argon followed by the addition of 1,1′-bis(di-t-butylphosphino)ferrocene palladium dichloride (8.8 mg, 0.013 mmol). The resulting solution was degassed again with argon and heated at 90° C. for 4 h. After cooled to ambient temperature, the resulting mixture was concentrated under reduced pressure to give the crude residue which was purified by silica gel 12 g column chromatography using 0-50% MeOH in DCM as eluent to give tert-butyl 4-(7-(2-((tert-butoxycarbonyl)amino)-7-fluorobenzo[d]thiazol-4-yl)-6-(1,1-difluoroethyl)-8-fluoro-2-((tetrahydro-1H-pyrrolizin-7a(5H)-yl)methoxy)quinazolin-4-yl)piperazine-1-carboxylate (25 mg, 35%). LCMS (ESI+): 802.57 [M+H]. ¹H NMR (301 MHz, CDCl₃) δ 7.88 (d, J=30.5 Hz, 1H), 7.29 (td, J=5.3, 2.6 Hz, 1H), 7.07 (t, J=8.7 Hz, 1H), 4.39-4.27 (m, 2H), 3.92-3.79 (m, 4H), 3.70-3.59 (m, 5H), 3.28 (s, 2H), 2.67 (s, 2H), 2.11 (td, J=12.4, 6.1 Hz, 3H), 1.98-1.63 (m, 8H), 1.51 (d, J=6.5 Hz, 18H).

Step D: Preparation of 4-(6-(1,1-difluoroethyl)-8-fluoro-4-(piperazin-1-yl)-2-((tetrahydro-1H-pyrrolizin-7a(5H)-yl)methoxy)quinazolin-7-yl)-7-fluorobenzo[d]thiazol-2-amine: To a solution of tert-butyl 4-(7-(2-((tert-butoxycarbonyl)amino)-7-fluorobenzo[d]thiazol-4-yl)-6-(1,1-difluoroethyl)-8-fluoro-2-((tetrahydro-1H-pyrrolizin-7a(5H)-yl)methoxy)quinazolin-4-yl)piperazine-1-carboxylate (25 mg, 0.031 mmol) in DCM (1 mL) was added trifluoroacetic acid (2 mL, 9.09 mmol) at 0° C. The mixture was stirred at room temperature for 2 h. The reaction mixture was evaporated and co-evaporated with DCM to give 4-(6-(1,1-difluoroethyl)-8-fluoro-4-(piperazin-1-yl)-2-((tetrahydro-1H-pyrrolizin-7a(5H)-yl)methoxy)quinazolin-7-yl)-7-fluorobenzo[d]thiazol-2-amine (0.032 g, 99%) as a TFA salt. LCMS (ESI+): 602.34. ¹H NMR (300 MHz, CD₃OD) δ 8.05 (d, J=21.1 Hz, 1H), 7.23 (ddd, J=8.0, 5.4, 2.3 Hz, 1H), 7.03 (dd, J=9.2, 8.5 Hz, 1H), 4.70 (s, 2H), 4.21 (t, J=5.3 Hz, 4H), 3.69 (dq, J=13.6, 6.1 Hz, 2H), 3.56-3.48 (m, 4H), 3.28 (d, J=6.6 Hz, 2H), 2.22 (dddt, J=43.6, 18.7, 12.0, 5.6 Hz, 8H), 1.66 (dd, J=19.3, 18.1 Hz, 3H).

### Synthetic Example 37: 4-(4-((1R,5S)-3,8-diazabicyclo[3.2.1]octan-3-yl)-6-(difluoromethyl)-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (Compound 301)

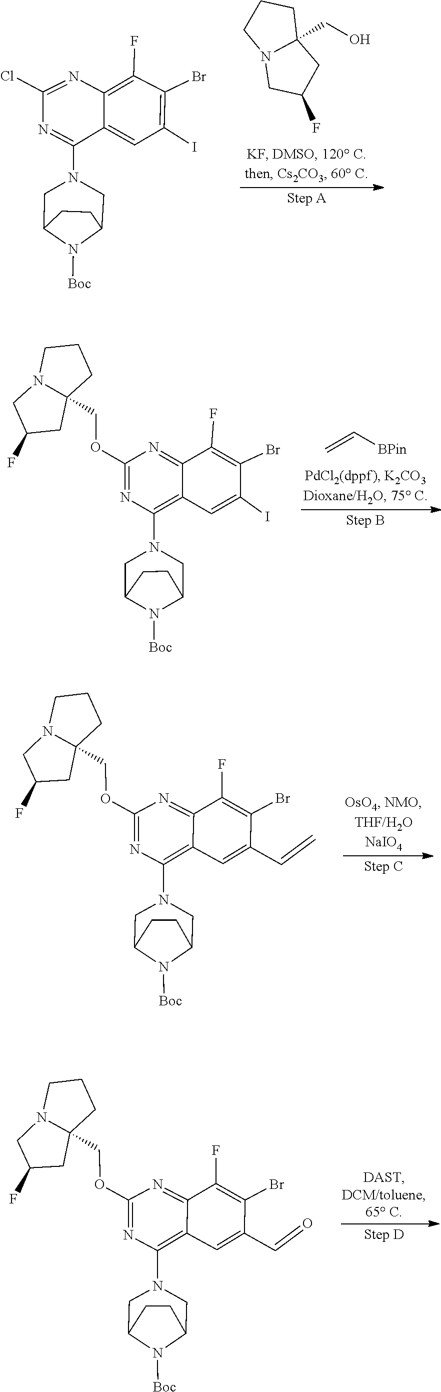

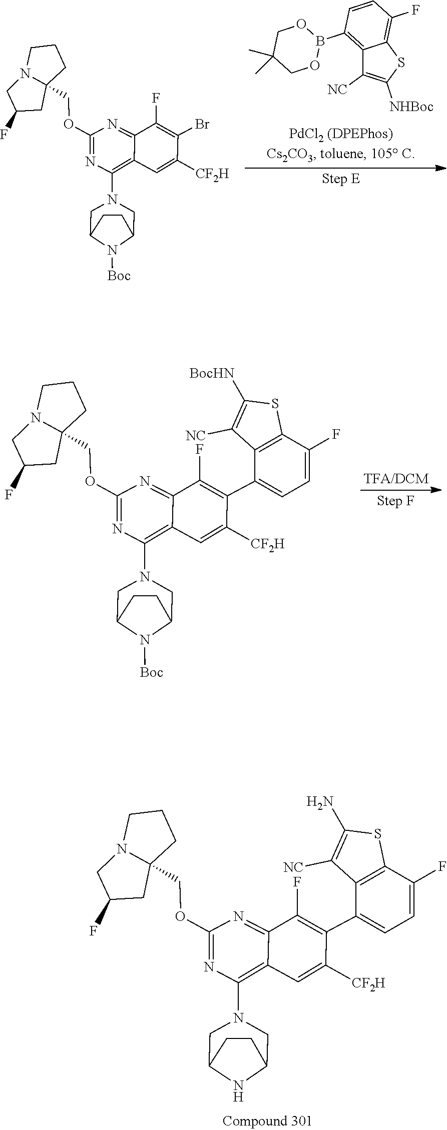 [SMILES: CC(C)(C)OC(=O)N1C2CCC1CN(c1nc(OC[C@@]34CCCN3C[C@H](F)C4)nc3c(F)c(Br)c(C(F)F)cc13)C2 ; CC1(C)COB(c2ccc(F)c3sc(NC(=O)OC(C)(C)C)c(C#N)c23)OC1 ; CC(C)(C)OC(=O)Nc1sc2c(F)ccc(-c3c(C(F)F)cc4c(N5CC6CCC(C5)N6C(=O)OC(C)(C)C)nc(OC[C@@]56CCCN5C[C@H](F)C6)nc4c3F)c2c1C#N ; N#Cc1c(N)sc2c(F)ccc(-c3c(C(F)F)cc4c(N5CC6CCC(C5)N6)nc(OC[C@@]56CCCN5C[C@H](F)C6)nc4c3F)c12]

Step A: Preparation of tert-butyl 3-[7-bromo-8-fluoro-2-[[(2R)-2-fluoro-1,2,3,5,6,7-hexahydropyrrolizin-8-yl]methoxy]-6-iodo-quinazolin-4-yl]-3,8-diazabicyclo[3.2.1]octane-8-carboxylate: The solution of tert-butyl 3-(7-bromo-2-chloro-8-fluoro-6-iodo-quinazolin-4-yl)-3,8-diazabicyclo[3.2.1]octane-8-carboxylate (100 mg, 0.17 mmol) and potassium fluoride (194 mg, 3.35 mmol) in DMSO (3.0 mL) was stirred at 120° C. for 18 h. Then it was cooled to rt and [(2R)-2-fluoro-1,2,3,5,6,7-hexahydropyrrolizin-8-yl]methanol (40 mg, 0.251 mmol) was added, followed by cesium carbonate (164 mg, 0.50 mmol). The reaction mixture was stirred at 60° C. for 4 h. After completion of the reaction, it was washed with sat. NH₄C₁ solution and extracted with EtOAc (50 mL). The organic layer was dried (Na₂SO₄), concentrated to give crude residue which was purified by silica gel column chromatography (40 g) using MeOH/DCM (0-20%) as eluent to give tert-butyl 3-[7-bromo-8-fluoro-2-[[(2R)-2-fluoro-1,2,3,5,6,7-hexahydropyrrolizin-8-yl]methoxy]-6-iodo-quinazolin-4-yl]-3,8-diazabicyclo[3.2.1]octane-8-carboxylate (62 mg, 51%). LCMS ESI (+) 720.3 [M+H]. ¹H NMR (300 MHz, cdcl₃) δ 8.06 (d, J=1.8 Hz, 1H), 5.26 (d, J=53.7 Hz, 1H), 4.35-4.22 (m, 3H), 4.21 (d, J=10.4 Hz, 1H), 4.10 (d, J=10.4 Hz, 1H), 3.70-3.40 (m, 3H), 3.38-3.06 (m, 3H), 2.97 (t, J=7.9 Hz, 1H), 2.35-2.06 (m, 6H), 1.99-1.85 (m, 4H), 1.50 (s, 9H).

Step B: Preparation of tert-butyl 3-[7-bromo-8-fluoro-2-[[(2R,8S)-2-fluoro-1,2,3,5,6,7-hexahydropyrrolizin-8-yl]methoxy]-6-vinyl-quinazolin-4-yl]-3,8-diazabicyclo[3.2.1]octane-8-carboxylate: A solution of tert-butyl 3-[7-bromo-8-fluoro-2-[[(2R,8S)-2-fluoro-1,2,3,5,6,7-hexahydropyrrolizin-8-yl]methoxy]-6-iodo-quinazolin-4-yl]-3,8-diazabicyclo[3.2.1]octane-8-carboxylate (165 mg, 0.23 mmol), potassium carbonate (63 mg, 0.46 mmol) in water (0.40 mL) and 1,4-dioxane (4.0 mL) was degassed with nitrogen for 5 minutes, followed by the addition of 1,1-bis(diphenylphosphino)ferrocene-palladium(II)dichloride dichloromethane complex (17 mg, 0.023 mmol). The mixture was degassed with nitrogen for additional 5 minutes and 4,4,5,5-tetramethyl-2-vinyl-1,3,2-dioxaborolane (0.058 mL, 0.34 mmol) was added. The reaction mixture was heated to 75° C. and stirred there for 4 h. The reaction mixture was extracted with EtOAc-NaCl. The EtOAc was collected, dried (Na₂SO₄), filtered and evaporated. The mixture was purified by silica gel column chromatography (40 g) using MeOH/DCM (0-10%) to provide tert-butyl 3-[7-bromo-8-fluoro-2-[[(2R,8S)-2-fluoro-1,2,3,5,6,7-hexahydropyrrolizin-8-yl]methoxy]-6-vinyl-quinazolin-4-yl]-3,8-diazabicyclo[3.2.1]octane-8-carboxylate (120 mg, 84%). LCMS ESI (+) m/z 620.29 (M+H). ¹H NMR (300 MHz, CDCl₃) δ 7.70 (d, J=1.8 Hz, 1H), 7.06 (dd, J=17.3, 10.9 Hz, 1H), 5.65 (dd, J=17.3, 0.9 Hz, 1H), 5.43-5.37 (m, 1H), 5.37-5.15 (m, 1H), 4.47-4.26 (m, 4H), 4.22 (d, J=10.3 Hz, 1H), 4.10 (d, J=10.3 Hz, 1H), 3.30-3.09 (m, 4H), 2.96 (td, J=9.1, 5.1 Hz, 1H), 2.31-2.03 (m, 5H), 2.00-1.84 (m, 6H), 1.50 (s, 9H).

Step C: Preparation of tert-butyl 3-[7-bromo-8-fluoro-2-[[(2R,8S)-2-fluoro-1,2,3,5,6,7-hexahydropyrrolizin-8-yl]methoxy]-6-formyl-quinazolin-4-yl]-3,8-diazabicyclo[3.2.1]octane-8-carboxylate: To a mixture of tert-butyl 3-[7-bromo-8-fluoro-2-[[(2R,8S)-2-fluoro-1,2,3,5,6,7-hexahydropyrrolizin-8-yl]methoxy]-6-vinyl-quinazolin-4-yl]-3,8-diazabicyclo[3.2.1]octane-8-carboxylate (120 mg, 0.19 mmol) in THE (4.0 mL) and water (1.0 mL) was added osmium tetroxide (0.0018 mL, 0.0058 mmol), followed by 4-methylmorpholine N-oxide (45 mg, 0.39 mmol) at room temperature. After 2 h (completion), sodium periodate (62 mg, 0.29 mmol) was added to the reaction and stirred for 2 h. After completion of the reaction (suspension formed), it was washed with water, brine and was extracted with EtOAc (60 mL). The organic layer was dried (Na₂SO₄) and concentrated to give crude residue which was purified by silica gel column chromatography (24 g) using MeOH/DCM (0-10%) as eluent to give tert-butyl 3-[7-bromo-8-fluoro-2-[[(2R,8S)-2-fluoro-1,2,3,5,6,7-hexahydropyrrolizin-8-yl]methoxy]-6-formyl-quinazolin-4-yl]-3,8-diazabicyclo[3.2.1]octane-8-carboxylate (80 mg, 66%). ¹H NMR (300 MHz, CDCl₃) δ 10.32 (s, 1H), 8.21 (d, J=1.7 Hz, 1H), 5.26 (dt, J=55.5, 2.8 Hz, 1H), 4.51-4.28 (m, 4H), 4.24 (d, J=10.3 Hz, 1H), 4.12 (d, J=10.3 Hz, 1H), 3.65 (dd, J=12.5, 5.0 Hz, 2H), 3.30-3.21 (m, 2H), 3.17-3.11 (m, 1H), 2.96 (td, J=9.3, 5.0 Hz, 1H), 2.27 (dd, J=9.4, 2.9 Hz, 1H), 2.22-2.11 (m, 2H), 1.99-1.85 (m, 5H), 1.69 (d, J=8.4 Hz, 2H), 1.51 (s, 9H).

Step D: Preparation of tert-butyl 3-[7-bromo-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate: To a solution of tert-butyl 3-(7-bromo-8-fluoro-6-formyl-2-methoxy-quinazolin-4-yl)-3,8-diazabicyclo[3.2.1]octane-8-carboxylate; (2R,8S)-2-fluoro-8-methyl-1,2,3,5,6,7-hexahydropyrrolizine (90 mg, 0.14 mmol) in DCM (1.0 mL) and toluene (1.0 mL) was added DAST (0.06 mL, 0.42 mmol). The mixture was heated at 65° C. for 3 h. The reaction was extracted with EtOAc, the EtOAc was collected, dried (Na₂SO₄), filtered and evaporated. The mixture was purified by silica gel column chromatography (24 g) using MeOH/DCM (0-10%) as eluent to provide tert-butyl 3-[7-bromo-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (35 mg, 38%). LCMS ESI (+) m/z 644.43 (M+H). ¹H NMR (300 MHz, CDCl₃) δ 7.84 (d, J=1.7 Hz, 1H), 6.93 (t, J=55.0 Hz, 1H), 5.28 (d, J=53.5 Hz, 1H), 4.42-4.32 (s, 4H), 4.25 (d, J=10.4 Hz, 1H), 4.18-4.11 (m, 1H), 3.60 (bs, 2H), 3.38-3.22 (m, 2H), 3.16 (d, J=2.9 Hz, 1H), 2.98 (td, J=9.2, 5.0 Hz, 1H), 2.33-2.23 (m, 1H), 2.23-2.12 (m, 2H), 2.10-1.85 (m, 5H), 1.79-1.68 (m, 2H), 1.51 (s, 9H).

Step E: Preparation of tert-butyl 3-[7-[2-(tert-butoxycarbonylamino)-3-cyano-7-fluoro-benzothiophen-4-yl]-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate: The solution of tert-butyl 3-[7-bromo-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (18 mg, 0.028 mmol), tert-butyl N-[3-cyano-7-(5,5-dimethyl-1,3,2-dioxaborinan-2-yl)-4-fluoro-benzothiophen-2-yl]carbamate (17 mg, 0.042 mmol) and cesium carbonate (23 mg, 0.0698 mmol) in toluene (1.0 mL) was sparged for 20 minutes at rt. Then dichlorobis(diphenylphosphinophenyl)ether palladium(II) (3.0 mg, 0.0042 mmol) was added and sparged with nitrogen for 5 minutes, stirred for 16 h at 105° C. Then the reaction mixture was cooled to rt and filtered through Celite®, and rinsed with EtOAc. The filtrate was evaporated under reduced pressure to give crude residue which was purified by silica gel column chromatography (12 g) using MeOH/DCM (0-20%) as eluent gave tert-butyl 3-[7-[2-(tert-butoxycarbonylamino)-3-cyano-7-fluoro-benzothiophen-4-yl]-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (11 mg) as an inseparable mixture with the des-bromo compound. LCMS ESI (+) m/z 856.52 (M+H).

Step F: Preparation of 2-amino-4-[4-(3,8-diazabicyclo[3.2.1]octan-3-yl)-6-(difluoromethyl)-8-fluoro-2-[[(2R,8S)-2-fluoro-1,2,3,5,6,7-hexahydropyrrolizin-8-yl]methoxy]quinazolin-7-yl]-7-fluoro-benzothiophene-3-carbonitrile: To the solution of tert-butyl 3-[7-[2-(tert-butoxycarbonylamino)-3-cyano-7-fluoro-benzothiophen-4-yl]-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (11 mg, 0.013 mmol) in DCM (1.0 mL) was added trifluoroacetic acid (0.50 mL, 6.49 mmol) at rt and stirred for 2 h. Then it was concentrated under reduced pressure to give residue which was purified by reverse phase chromatography using ACN/water as an eluent to give 2-amino-4-[4-(3,8-diazabicyclo[3.2.1]octan-3-yl)-6-(difluoromethyl)-8-fluoro-2-[[(2R,8S)-2-fluoro-1,2,3,5,6,7-hexahydropyrrolizin-8-yl]methoxy]quinazolin-7-yl]-7-fluoro-benzothiophene-3-carbonitrile (3.4 mg, 26%). LCMS ESI (+) m/z 656.32 (M+H). ¹H NMR (300 MHz, CD₃OD) δ 8.05 (s, 1H), 7.23 (dd, J=8.4, 5.0 Hz, 1H), 7.05 (dd, J=9.5, 8.4 Hz, 1H), 6.56 (t, J=54.8 Hz, 1H), 5.43 (d, J=52.9 Hz, 1H), 4.67-4.52 (m, 2H), 4.53-4.40 (m, 2H), 3.97-3.89 (m, 2H), 3.85-3.67 (m, 3H), 3.64-3.51 (m, 3H), 2.55-2.35 (m, 2H), 2.32-2.11 (m, 4H), 1.97 (s, 4H).

### Synthetic Example 38: 4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (Compound 260)

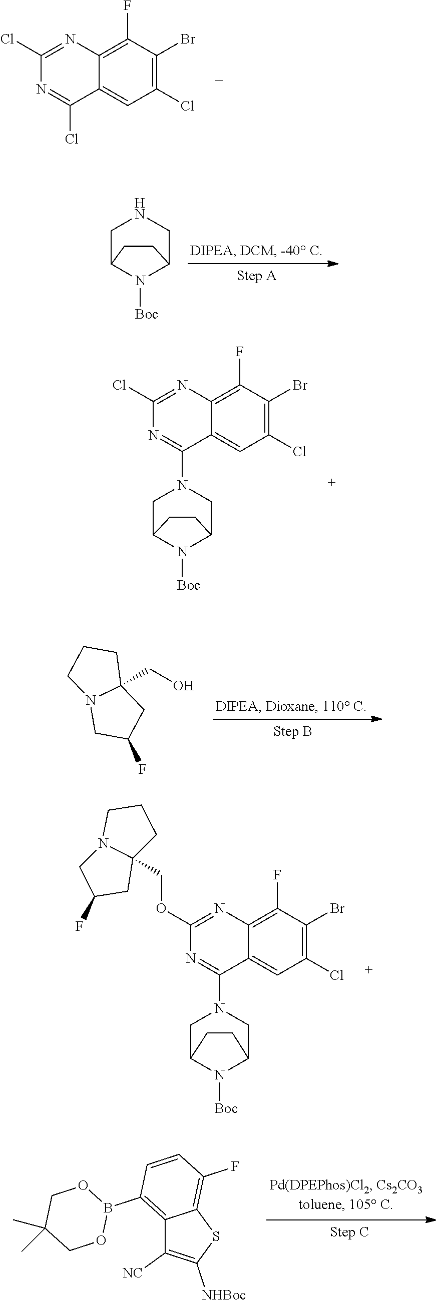

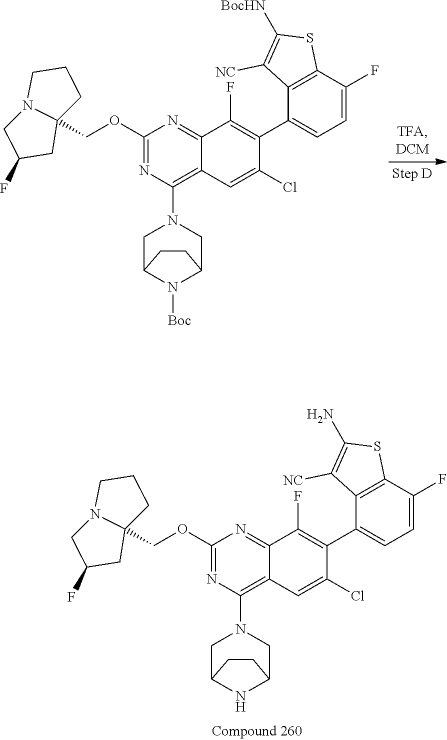

Step A: Preparation of tert-butyl 3-(7-bromo-2,6-dichloro-8-fluoro-quinazolin-4-yl)-3,8-diazabicyclo[3.2.1]octane-8-carboxylate: A solution of 7-bromo-2,4,6-trichloro-8-fluoroquinazoline (660 mg, 2.00 mmol) in DCM (10 mL) was stirred at −40° C. under argon. To this, DIPEA (1.0 mL, 5.99 mmol) and tert-butyl 3,8-diazabicyclo[3.2.1]octane-8-carboxylate (509 mg, 2.40 mmol) were added subsequently and stirred for 30 minutes at −40° C. After completion of the reaction, it was washed with sat. NH₄C₁ solution and extracted with EtOAc (50 mL). The organic layer was further washed with brine, dried (Na₂SO₄), concentrated to give crude residue which was purified by silica gel column (40 g) chromatography using EtOAc/hexanes (0-50%) as eluent to give tert-butyl 3-(7-bromo-2,6-dichloro-8-fluoro-quinazolin-4-yl)-3,8-diazabicyclo[3.2.1]octane-8-carboxylate (380 mg, 37%). ¹H NMR (300 MHz, CDCl₃) δ 7.75 (d, J=2.1 Hz, 1H), 4.50-4.25 (m, 4H), 3.87-3.40 (m, 2H), 2.01-1.88 (m, 2H), 1.82-1.65 (m, 2H), 1.52 (s, 9H).

Step B: Preparation of tert-butyl 3-[7-bromo-6-chloro-8-fluoro-2-[[(2R)-2-fluoro-1,2,3,5,6,7-hexahydropyrrolizin-8-yl]methoxy]quinazolin-4-yl]-3,8-diazabicyclo[3.2.1]octane-8-carboxylate: A solution of tert-butyl 3-(7-bromo-2,6-dichloro-8-fluoro-quinazolin-4-yl)-3,8-diazabicyclo[3.2.1]octane-8-carboxylate (380 mg, 0.75 mmol), [(2R)-2-fluoro-1,2,3,5,6,7-hexahydropyrrolizin-8-yl]methanol (179 mg, 1.13 mmol) and DIPEA (0.39 mL, 2.25 mmol) in 1,4-dioxane (6.0 mL) was heated to 120° C. for 3 days. Then it was washed with sat. NH₄C₁ solution (20 mL), followed by brine. The organic layer was extracted with EtOAc (50 mL). The organic layer was dried (Na₂SO₄) and concentrated to give crude residue which was purified by flash column chromatography (40 g) eluting with MeOH/DCM (0-20%). The desired fractions were concentrated to dryness in vacuo to provide tert-butyl 3-[7-bromo-6-chloro-8-fluoro-2-[[(2R)-2-fluoro-1,2,3,5,6,7-hexahydropyrrolizin-8-yl]methoxy]quinazolin-4-yl]-3,8-diazabicyclo[3.2.1]octane-8-carboxylate (100 mg, 21% yield). ¹H NMR (300 MHz, CDCl₃) δ 7.67 (d, J=2.1 Hz, 1H), 5.39-5.12 (m, 1H), 4.41-4.26 (m, 4H), 4.21 (d, J=10.3 Hz, 1H), 4.11 (d, J=2.1 Hz, 1H), 3.64-3.44 (m, 2H), 3.31-3.20 (m, 2H), 3.18-3.09 (m, 1H), 3.03-2.90 (m, 1H), 2.32-2.22 (m, 1H), 2.22-2.06 (m, 2H), 1.91 (q, J=7.4 Hz, 4H), 1.78-1.64 (m, 3H), 1.51 (s, 9H).

Step C: Preparation of tert-butyl 3-[7-[2-(tert-butoxycarbonylamino)-3-cyano-7-fluoro-benzothiophen-4-yl]-6-chloro-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate: The solution of tert-butyl 3-[7-bromo-6-chloro-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (50 mg, 0.079 mmol), tert-butyl N-[3-cyano-7-(5,5-dimethyl-1,3,2-dioxaborinan-2-yl)-4-fluoro-benzothiophen-2-yl]carbamate (48 mg, 0.20 mmol) and cesium carbonate (65 mg, 0.20 mmol) in toluene (2.50 mL) was sparged for 20 minutes at rt. Then, dichlorobis(diphenylphosphinophenyl)ether palladium(II) (8.50 mg, 0.012 mmol) was added and sparged for another 5 minutes. Then the reaction mixture was stirred for 16 h at 105° C. After that the reaction mixture was cooled to rt, filtered through Celite® and rinsed with EtOAc. The filtrate was evaporated under reduced pressure to give crude residue which was purified by flash silica gel column chromatography (24 g) using EtOAc/hexanes (0-100%) followed by MeOH/DCM (0-10%) as eluent to give the desired material along with the des-chloro compound. The obtained material was again purified by reverse phase chromatography (24 g) using ACN/0.1% HCOOH buffer (5-95%) as eluent to give tert-butyl 3-[7-[2-(tert-butoxycarbonylamino)-3-cyano-7-fluoro-benzothiophen-4-yl]-6-chloro-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (16 mg, 24%). LCMS ESI (+) m/z 840.48 (M+H). ¹H NMR (300 MHz, (CD₃)₂C(O)) δ 8.14 (s, 1H), 7.92 (d, J=1.8 Hz, 1H), 7.46 (dd, J=8.3, 5.1 Hz, 1H), 7.34 (dd, J=9.5, 8.3 Hz, 1H), 5.32 (d, J=54.1 Hz, 1H), 4.46 (t, J=13.6 Hz, 2H), 4.35 (s, 2H), 4.26-4.16 (m, 2H), 3.68 (dd, J=21.0, 12.4 Hz, 2H), 3.33-3.11 (m, 3H), 3.00-2.89 (m, 1H), 2.33-2.10 (m, 3H), 1.96-1.78 (m, 7H), 1.53 (s, 9H), 1.50 (s, 9H).

Step D: Preparation of 4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To the solution of tert-butyl 3-[7-[2-(tert-butoxycarbonylamino)-3-cyano-7-fluoro-benzothiophen-4-yl]-6-chloro-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (15 mg, 0.018 mmol) in DCM (1.0 mL) was added trifluoroacetic acid (0.50 mL, 6.44 mmol) at rt and stirred for 2 h. Then it was concentrated under reduced pressure to give residue which was triturated with diethyl ether followed by DCM and dried to give 2-amino-4-[6-chloro-4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]quinazolin-7-yl]-7-fluoro-benzothiophene-3-carbonitrile (12 mg, 68%). LCMS ESI (+) m/z 640.35 (M+H). ¹H NMR (300 MHz, CD₃OD) δ 7.96 (d, J=1.7 Hz, 1H), 7.22 (dd, J=8.4, 5.1 Hz, 1H), 7.06 (dd, J=9.5, 8.4 Hz, 1H), 5.58 (d, J=48.0 Hz, 1H), 4.77-4.62 (m, 4H), 4.24 (s, 2H), 4.01-3.86 (m, 5H), 3.48-3.41 (m, 1H), 2.74-2.54 (m, 2H), 2.43-2.27 (m, 3H), 2.22-2.10 (m, 5H).

### Synthetic Example 39: Synthesis of 4-(4-((1R,5S)-8-ethyl-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 315)

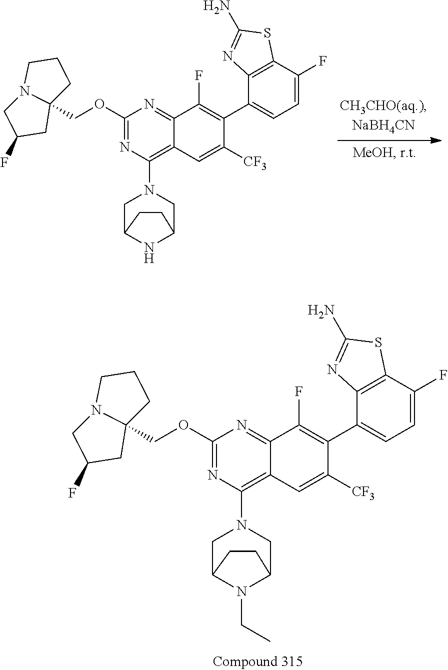 [SMILES: Nc1nc2c(-c3c(C(F)(F)F)cc4c(N5CC6CCC(C5)N6)nc(OC[C@@]56CCCN5C[C@H](F)C6)nc4c3F)ccc(F)c2s1 ; CCN1C2CCC1CN(c1nc(OC[C@@]34CCCN3C[C@H](F)C4)nc3c(F)c(-c4ccc(F)c5sc(N)nc45)c(C(F)(F)F)cc13)C2]

Preparation of 4-(4-((1R,5S)-8-ethyl-3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine: To a mixture of 4-[4-[(1R,5S)-3,8-diazabicyclo[3.2.1]octan-3-yl]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-1,3-benzothiazol-2-amine (30 mg) in methanol (1 mL) was added acetaldehyde (aq. solution) (0.15 mL, 2.66 mmol) at ambient temperature and stirred for 1 hour, then NaBH₃CN (7.3 mg, 0.115 mmol) was added and stirred at ambient temperature for 2 hours. The reaction was concentrated under reduced pressure and the residue was purified by Preparative RP-HPLC to give 4-[4-[(1R,5S)-8-ethyl-3,8-diazabicyclo[3.2.1]octan-3-yl]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-1,3-benzothiazol-2-amine (4.6 mg, 29%). LCMS ESI (+) m/z 678.24 (M+H). ¹HNMR (400 MHz, CD₃OD) δ 8.17 (s, 1H), 7.17-7.24 (m, 1H), 7.00 (t, J=8.8 Hz, 1H), 5.57 (d, J=52 Hz, 1H), 4.78-4.83 (m, 3H), 4.64-4.75 (m, 2H), 4.19-4.32 (m, 2H), 3.82-4.10 (m, 5H), 3.42-3.52 (m, 1H), 3.17-3.27 (m, 1H), 2.54-2.76 (m, 2H), 2.08-2.50 (m, 8H), 1.40-1.52 (m, 3H).

### Synthetic Example 40: Synthesis of 4-(4-((1R,5S)-3,8-diazabicyclo[3.2.1]octan-3-yl)-8-chloro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine (Compound 256)

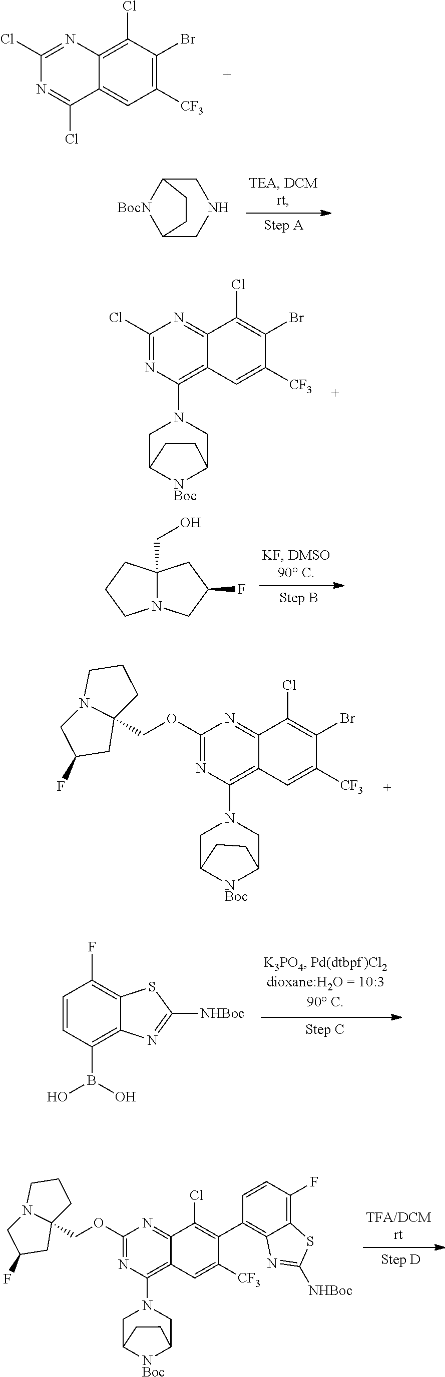

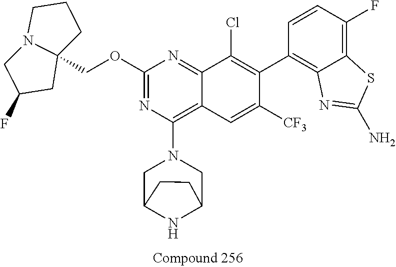 [SMILES: Nc1nc2c(-c3c(C(F)(F)F)cc4c(N5CC6CCC(C5)N6)nc(OC[C@@]56CCCN5C[C@H](F)C6)nc4c3Cl)ccc(F)c2s1]

Step A: Preparation of tert-butyl (1R,5S)-3-(7-bromo-2,8-dichloro-6-(trifluoromethyl)quinazolin-4-yl)-3,8-diazabicyclo[3.2.1]octane-8-carboxylate: To a mixture of 7-bromo-2,4,8-trichloro-6-(trifluoromethyl)quinazoline (150 mg, 0.39 mmol) in DCM (3 mL) was added tert-butyl 3,8-diazabicyclo[3.2.1]octane-8-carboxylate (84 mg, 0.39 mmol) and triethylamine (0.16 mL, 1.18 mmol) at room temperature and stirred at room temperature for 1 hour. The reaction was concentrated to dryness and the residue was taken up in EtOAc (30 mL) and the organic layers washed with water and saturated brine solution. The organic layers were then separated and dried (MgSO₄), filtered and concentrated under reduced pressure. The crude was then purified by Preparative-TLC (25% EtOAc in petroleum ether) to give tert-butyl 3-[7-bromo-2,8-dichloro-6-(trifluoromethyl)quinazolin-4-yl]-3,8-diazabicyclo[3.2.1]octane-8-carboxylate (78 mg, 35%).

Step B: Preparation of tert-butyl 3-[7-bromo-8-chloro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate: To a solution of tert-butyl 3-[7-bromo-2,8-dichloro-6-(trifluoromethyl)quinazolin-4-yl]-3,8-diazabicyclo[3.2.1]octane-8-carboxylate (78 mg, 0.14 mmol) in DMSO (5 mL) was added ((2R,7aS)-2-fluorotetrahydro-1H-pyrrolizin-7a(5H)-yl)methanol (27 mg, 0.17 mmol) and KF (65 mg, 1.13 mmol) at ambient temperature. The mixture was stirred at 90° C. for 3 hours. After cooled to ambient temperature, ethyl acetate and brine were added. The organic layers were then separated and dried over Na₂SO₄, and filtered before concentration to dryness. The crude product was purified by Preparative-TLC (EtOAc) to give tert-butyl 3-[7-bromo-8-chloro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (60 mg, 62%). LCMS (ES+) m/z 678.2 (M+H).

Step C: Preparation of tert-butyl 3-[7-[2-(tert-butoxycarbonylamino)-7-fluoro-1,3-benzothiazol-4-yl]-8-chloro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate: A mixture of tert-butyl 3-[7-bromo-8-chloro-2-[[(2S,8R)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (60 mg, 0.088 mmol), [2-(tert-butoxycarbonylamino)-7-fluoro-1,3-benzothiazol-4-yl]boronic acid (36 mg, 0.12 mmol), Pd(dtbpf)Cl₂ (5.8 mg, 0.0089 mmol), and K₃PO₄ (38 mg, 0.18 mmol) in 1,4-dioxane (1 mL)/water (0.20 mL) was stirred at 90° C. under Ar for 2 hours. After cooled to ambient temperature, the reaction was concentrated to dryness and the residue was taken up in EtOAc (20 mL) and the organic layers washed with saturated brine solution. The organic layers were then separated, dried (MgSO₄), filtered and concentrated under reduced pressure. The crude was then purified by preparative-TLC (25% EtOAc in petroleum ether) to give tert-butyl 3-[7-[2-(tert-butoxycarbonylamino)-7-fluoro-1,3-benzothiazol-4-yl]-8-chloro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (40 mg, 52% yield). LCMS (ES+) m/z 866.2 (M+H).

Step D: Preparation of 4-[8-chloro-4-(3,8-diazabicyclo[3.2.1]octan-3-yl)-2-[[(2R,8S)-2-fluoro-1,2,3,5,6,7-hexahydropyrrolizin-8-yl]methoxy]-6-(trifluoromethyl)quinazolin-7-yl]-7-fluoro-1,3-benzothiazol-2-amine: To a solution of tert-butyl 3-[7-[2-(tert-butoxycarbonylamino)-7-fluoro-1,3-benzothiazol-4-yl]-8-chloro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (1.00 eq, 45 mg, 0.0518 mmol) in DCM (3 mL) was added TFA (1.5 mL, 19.5 mmol) at ambient temperature and stirred at this temperature for 2 hours. The reaction was concentrated under reduced pressure. The residue obtained was then purified by preparative RP-HPLC to give 4-[8-chloro-4-(3,8-diazabicyclo[3.2.1]octan-3-yl)-2-[[(2R,8S)-2-fluoro-1,2,3,5,6,7-hexahydropyrrolizin-8-yl]methoxy]-6-(trifluoromethyl)quinazolin-7-yl]-7-fluoro-1,3-benzothiazol-2-amine (5.3 mg, 14%). LCMS ESI (+) m/z 666.3 (M+H). ¹HNMR (400 MHz, CD₃OD) δ 8.30 (s, 1H), 7.07-7.18 (m, 1H), 7.00 (t, J=8.8 Hz, 1H), 5.56 (d, J=52.8 Hz, 1H), 4.60-4.83 (m, 3H), 4.25 (s, 2H), 3.85-4.10 (m, 5H), 3.41-3.52 (m, 2H), 2.56-2.80 (m, 2H), 2.30-2.52 (m, 3H), 1.83-2.23 (m, 5H).

### Synthetic Example 41: Synthesis of 7-fluoro-4-(8-fluoro-2-(((2R,7aS)-2-fluorotetrahydro-1H-pyrrolizin-7a(5H)-yl)methoxy)-4-(methyl((R)-pyrrolidin-3-yl)amino)-6-(trifluoromethyl)quinazolin-7-yl)benzo[d]thiazol-2-amine (Compound 322)

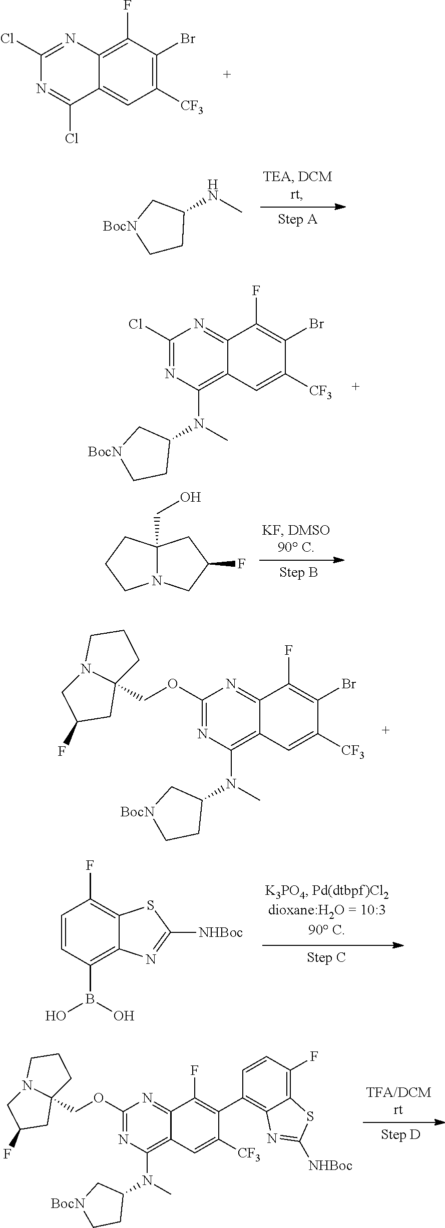

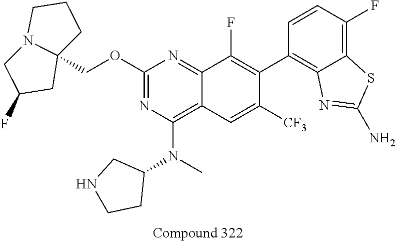 [SMILES: CN(c1nc(OC[C@@]23CCCN2C[C@H](F)C3)nc2c(F)c(-c3ccc(F)c4sc(N)nc34)c(C(F)(F)F)cc12)[C@@H]1CCNC1]

Step A: Preparation of tert-butyl (R)-3-((7-bromo-2-chloro-8-fluoro-6-(trifluoromethyl)quinazolin-4-yl)(methyl)amino)pyrrolidine-1-carboxylate: To a mixture of 7-bromo-2,4-dichloro-8-fluoro-6-(trifluoromethyl)quinazoline (150 mg, 0.41 mmol) in DCM (1.5 mL) was added tert-butyl (3R)-3-(methylamino)pyrrolidine-1-carboxylate (91 mg, 0.453 mmol) and triethylamine (0.17 mL, 1.24 mmol) at room temperature and stirred at room temperature for 1 hour. The reaction was concentrated to dryness and the residue was taken up in EtOAc (30 mL) and the organic layers washed with water, saturated brine solution. The organic layers were then separated and dried (MgSO₄), filtered and concentrated under reduced pressure. The crude was then purified by Preparative-TLC (25% EtOAc in petroleum ether) to tert-butyl (R)-3-((7-bromo-2-chloro-8-fluoro-6-(trifluoromethyl)quinazolin-4-yl)(methyl)amino)pyrrolidine-1-carboxylate (88 mg, 44%).

Step B: Preparation of tert-butyl (3R)-3-[[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]pyrrolidine-1-carboxylate: To a solution of tert-butyl (3R)-3-[[7-bromo-2-chloro-8-fluoro-6-(trifluoromethyl)quinazolin-4-yl]-methyl-amino]pyrrolidine-1-carboxylate (50 mg, 0.095 mmol) in DMSO (1 mL) were added ((2R,7aS)-2-fluorotetrahydro-1H-pyrrolizin-7a(5H)-yl)methanol (23 mg, 0.14 mmol) and KF (44 mg, 0.76 mmol) at ambient temperature. The mixture was stirred at 90° C. for 4 hours. After cooled to ambient temperature, ethyl acetate and brine were added. The organic layers were then separated and dried over Na₂SO₄, filtered before concentration to dryness. The crude product was purified by Preparative-TLC (EtOAc) to give tert-butyl (3R)-3-[[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]pyrrolidine-1-carboxylate (25 mg, 40%). LCMS (ES+) m/z 651.2 (M+H).

Step C: Preparation of tert-butyl (3R)-3-[[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]pyrrolidine-1-carboxylate: A mixture of tert-butyl (3R)-3-[[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]pyrrolidine-1-carboxylate (80 mg, 0.12 mmol), [2-(tert-butoxycarbonylamino)-7-fluoro-1,3-benzothiazol-4-yl]boronic acid (38 mg, 0.12 mmol), K₃PO₄ (26 mg, 0.12 mmol), and Pd(dtbpf)Cl₂ (8.0 mg, 0.012 mmol) in 1,4-dioxane (1 mL)/water (0.20 mL) was stirred at 90° C. under Ar for 2 hours. After cooled to ambient temperature, the reaction was concentrated to dryness and the residue was taken up in EtOAc (20 mL) and the organic layers washed with saturated brine solution. The organic layers were then separated, dried (MgSO₄), filtered and concentrated under reduced pressure. The crude was then purified by preparative-TLC (ethyl acetate) to give tert-butyl (3R)-3-[[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]pyrrolidine-1-carboxylate (80 mg).

Step D: Preparation of 7-fluoro-4-[8-fluoro-2-[[(2R,8S)-2-fluoro-1,2,3,5,6,7-hexahydropyrrolizin-8-yl]methoxy]-4-[methyl-[(3R)-pyrrolidin-3-yl]amino]-6-(trifluoromethyl)quinazolin-7-yl]-1,3-benzothiazol-2-amine: To a solution of tert-butyl (3R)-3-[[7-[2-(tert-butoxycarbonylamino)-7-fluoro-1,3-benzothiazol-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]pyrrolidine-1-carboxylate (44 mg, 0.053 mmol) in DCM (3 mL) was added TFA (1.5 mL, 19.5 mmol) at ambient temperature and stirred at this temperature for 2 hours. The reaction was concentrated under reduced pressure. The residue obtained was then purified by preparative RP-HPLC to give 7-fluoro-4-[8-fluoro-2-[[(2R,8S)-2-fluoro-1,2,3,5,6,7-hexahydropyrrolizin-8-yl]methoxy]-4-[methyl-[(3R)-pyrrolidin-3-yl]amino]-6-(trifluoromethyl)quinazolin-7-yl]-1,3-benzothiazol-2-amine (26 mg, 76%). LCMS ESI (+) m/z 638.3 (M+H). ¹HNMR (400 MHz, CD₃OD) δ 8.37 (s, 1H), 7.22 (dd, J=8.4, 5.6 Hz, 1H), 7.01 (t, J=8.8 Hz, 1H), 5.55 (d, J=52.0 Hz, 1H), 5.28-5.37 (m, 1H), 4.55-4.79 (m, 2H), 3.81-4.06 (m, 4H), 3.61-3.73 (m, 1H), 3.54 (s, 3H), 3.37-3.51 (m, 3H), 2.52-2.77 (m, 3H), 2.26-2.49 (m, 4H), 2.09-2.12 (m, 1H).

### Synthetic Example 42: Synthesis of 4-(4-(3,8-diazabicyclo[3.2.1]octan-3-yl)-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 323)

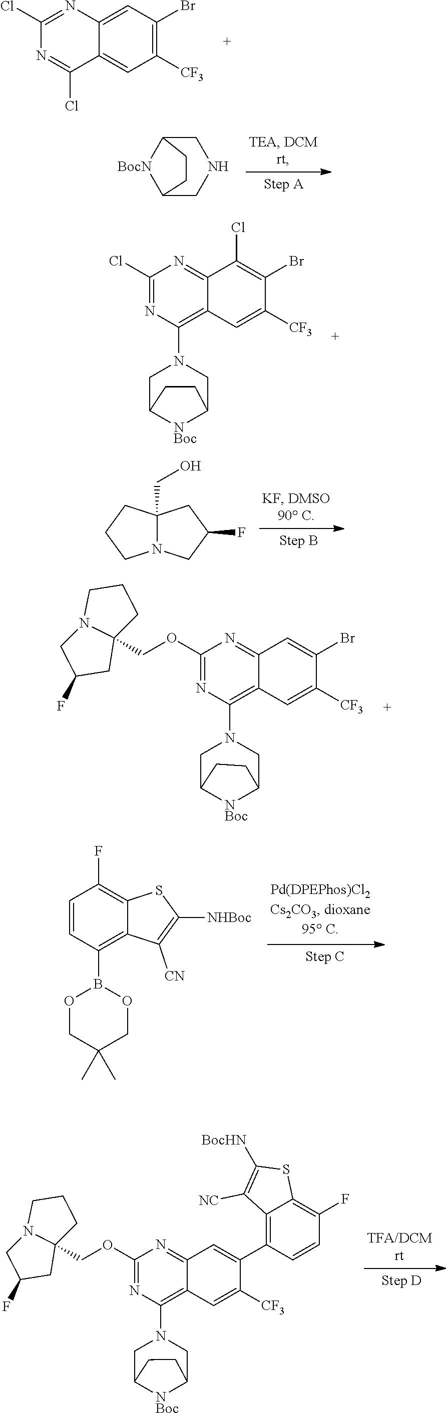

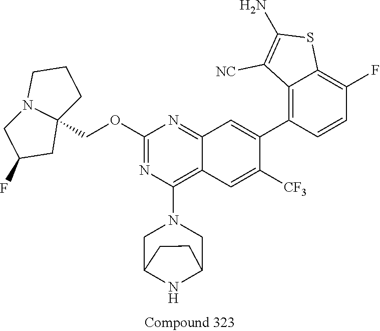 [SMILES: N#Cc1c(N)sc2c(F)ccc(-c3cc4nc(OC[C@@]56CCCN5C[C@H](F)C6)nc(N5CC6CCC(C5)N6)c4cc3C(F)(F)F)c12]

Step A: Preparation of tert-butyl (1R,5S)-3-(7-bromo-2-chloro-6-(trifluoromethyl)quinazolin-4-yl)-3,8-diazabicyclo[3.2.1]octane-8-carboxylate: A solution of 7-bromo-2,4-dichloro-6-(trifluoromethyl)quinazoline (800 mg, 2.31 mmol), tert-butyl 3,8-diazabicyclo[3.2.1]octane-8-carboxylate (539 mg, 2.54 mmol), and DIEA (12.7 mL, 6.93 mmol) in DCM (15 mL) was stirred at rt for 2 hours. The reaction mixture was quenched by water and extracted with DCM, the organic layer was washed with water and brine, dried over Na₂SO₄, filtered and concentrated under reduced pressure. The solvent was removed by rotary evaporation. The product was purified by column chromatography on silica gel to afford tert-butyl (1R,5S)-3-(7-bromo-2-chloro-6-(trifluoromethyl)quinazolin-4-yl)-3,8-diazabicyclo[3.2.1]octane-8-carboxylate (710 mg, 59% yield). LCMS ESI (+) m/z 521.0 (M+H).

Step B: Preparation of tert-butyl (1R,5S)-3-(7-bromo-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate: A solution of tert-butyl 3-[7-bromo-8-chloro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (710 mg, 1.36 mmol), ((2R,7aS)-2-fluorotetrahydro-1H-pyrrolizin-7a(5H)-yl)methanol (325 mg, 2.04 mmol) and KF (631 mg, 10.88 mmol) in DMSO (4 mL) was stirred at 90° C. for 4 hours. After cooled to ambient temperature, ethyl acetate and brine were added. The organics were then separated and dried over Na₂SO₄, filtered before concentration to dryness. The crude product was purified by Preparative-TLC to give tert-butyl (1R,5S)-3-(7-bromo-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (580 mg, 66%). LCMS (ES+) m/z 644.2 (M+H).

Step C: Preparation of tert-butyl 3-(7-(2-((tert-butoxycarbonyl)amino)-3-cyano-7-fluorobenzo[b]thiophen-4-yl)-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate: A solution of tert-butyl (1R,5S)-3-(7-bromo-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (420 mg, 0.65 mmol), tert-butyl (3-cyano-4-(5,5-dimethyl-1,3,2-dioxaborinan-2-yl)-7-fluorobenzo[b]thiophen-2-yl)carbamate (656.5 mg, 1.63 mmol), Cs₃CO₃ (423 mg, 1.3 mmol) and Pd(DPEPhos)Cl₂ (92.9 mg, 0.13 mmol) in dioxane (16 mL) was stirred at 90° C. for 16 hours. After cooled to ambient temperature, the reaction was concentrated to dryness and the residue was taken up in EtOAc (20 mL) and the organic layers washed with saturated brine solution. The organic layers were then separated, dried (MgSO₄), filtered and concentrated under reduced pressure. The crude was then purified by chromatography on silica gel to give tert-butyl 3-(7-(2-((tert-butoxycarbonyl)amino)-3-cyano-7-fluorobenzo[b]thiophen-4-yl)-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (430 mg, 77%). LCMS (ES+) m/z 856.3 (M+H).

Step D: Preparation of 4-(4-(3,8-diazabicyclo[3.2.1]octan-3-yl)-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile: A solution of tert-butyl 3-(7-(2-((tert-butoxycarbonyl)amino)-3-cyano-7-fluorobenzo[b]thiophen-4-yl)-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (430 mg, 0.50 mmol) in DCM/TFA (4 mL/1 mL) was stirred at room temperature for 2 hours. The solvent was removed by rotary evaporation and purified by Preparative RP-HPLC to give 4-(4-(3,8-diazabicyclo[3.2.1]octan-3-yl)-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (330 mg, 66%). LCMS ESI (+) m/z 656.3 (M+H). ¹HNMR (400 MHz, CD₃OD) δ 8.32 (s, 1H), 7.60 (s, 1H), 7.17-7.21 (m, 1H), 6.99 (t, J=8.8 Hz, 1H), 5.58 (d, J=52.8 Hz, 1H), 4.79-4.85 (m, 1H), 4.61-4.72 (m, 3H), 4.23-4.24 (m, 2H), 3.85-4.01 (m, 5H), 3.47-3.50 (m, 1H), 2.58-2.71 (m, 2H), 2.32-2.46 (m, 3H), 2.10-2.19 (m, 5H).

### Synthetic Example 43: Synthesis of 7-(2-amino-7-fluorobenzo[d]thiazol-4-yl)-4-((1R,5S)-3,8-diazabicyclo[3.2.1]octan-3-yl)-2-(((2R,7aS)-2-fluorotetrahydro-1H-pyrrolizin-7a(5H)-yl)methoxy)-6-(trifluoromethyl)quinazolin-8-ol (Compound 343)

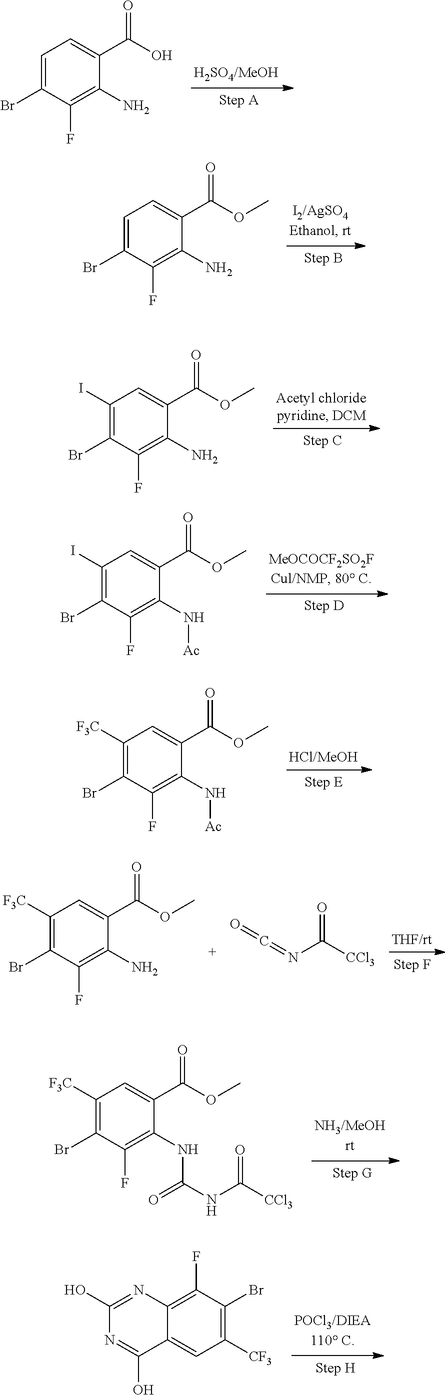

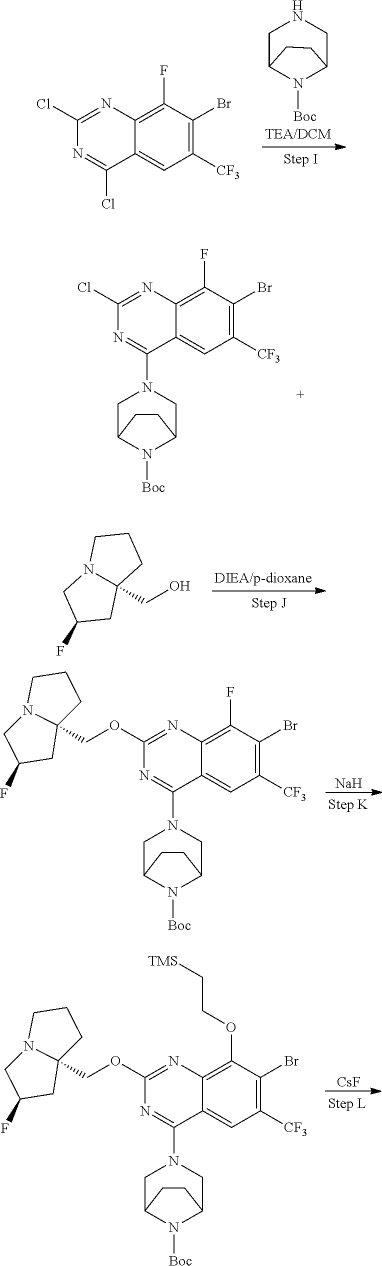

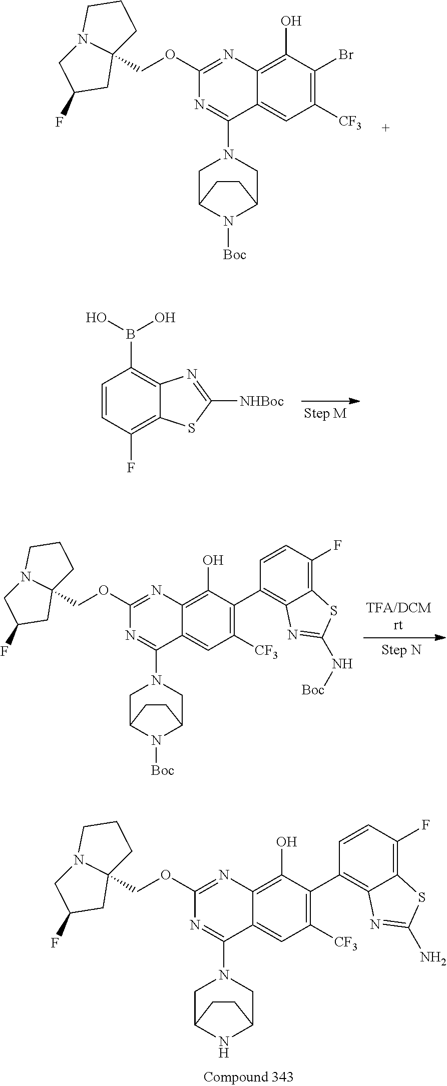 [SMILES: CC(C)(C)OC(=O)N1C2CCC1CN(c1nc(OC[C@@]34CCCN3C[C@H](F)C4)nc3c(O)c(Br)c(C(F)(F)F)cc13)C2 ; CC(C)(C)OC(=O)Nc1nc2c(B(O)O)ccc(F)c2s1 ; CC(C)(C)OC(=O)Nc1nc2c(-c3c(C(F)(F)F)cc4c(N5CC6CCC(C5)N6C(=O)OC(C)(C)C)nc(OC[C@@]56CCCN5C[C@H](F)C6)nc4c3O)ccc(F)c2s1 ; Nc1nc2c(-c3c(C(F)(F)F)cc4c(N5CC6CCC(C5)N6)nc(OC[C@@]56CCCN5C[C@H](F)C6)nc4c3O)ccc(F)c2s1]

Step A: Preparation of methyl 2-amino-4-bromo-3-fluoro-benzoate: To stirring solution of 2-amino-4-bromo-3-fluoro-benzoic acid (1.00 eq, 420.00 g, 1795 mmol) in methanol (4 L) was added dropwise H₂SO₄ (8.68 eq, 800 mL, 15584 mmol) at 30° C. The resulting mixture was heated to 100° C. for 5 hours. The solvent was evaporated, and water was added to the residue. The solid was filtered, the filter cake dissolved in DCM, washed with saturated sodium bicarbonate aqueous solution, and the organic phase was dried with Na₂SO₄, filtered, and concentrated under vacuum to give methyl 2-amino-4-bromo-3-fluoro-benzoate (400 g, 1532 mmol, 85% yield) as a solid. LCMS ESI (+) m/z 248 (M+H). ¹HNMR (400 MHz, CDCl₃) δ 7.53 (dd, J=8.8, 1.6 Hz, 1H), 6.78 (dd, J=8.8, 6.0 Hz, 1H), 3.90 (s, 3H).

Step B: Preparation of methyl 2-amino-4-bromo-3-fluoro-5-iodo-benzoate: To a mixture of iodine (1.40 eq, 573.43 g, 2258 mmol) and silver sulfate (0.850 eq, 427.38 g, 1371 mmol) in ethanol (15 L), methyl 2-amino-4-bromo-3-fluoro-benzoate (1.00 eq, 400.00 g, 1613 mmol) was added and the resulting mixture was stirred at ambient temperature for 45 minutes. The solid was filtered off and washed with DCM, and the filtrate was concentrated under vacuum. The residue was dissolved in DCM and washed with 10% sodium thiosulphate solution and brine and the resulting organic solution was dried over Na₂SO₄, filtered, and concentrated under vacuum to give methyl 2-amino-4-bromo-3-fluoro-5-iodo-benzoate (550.00 g, 89% yield) as a yellow solid. LCMS ESI (+) m/z 374 (M+H). ¹HNMR (400 MHz, CDCl₃) δ 8.14 (d, J=2 Hz, 1H), 5.94 (s, 2H), 3.91 (s, 3H).

Step C: Preparation of methyl 2-acetamido-4-bromo-3-fluoro-5-iodo-benzoate: The methyl 2-amino-4-bromo-3-fluoro-5-iodo-benzoate (1.00 eq, 430.00 g, 1150 mmol) and pyridine (3.00 eq, 272.52 g, 3450 mmol) were dissolved in DCM (5 L) at 0° C. Acetyl chloride (1.20 eq, 109.01 g, 1380 mmol) was added and the reaction was warmed to ambient temperature and stirred at this temperature for 2 hours. the reaction mixture was washed with 1N hydrochloric acid to pH<7, then washed with saturated sodium bicarbonate to pH>8, the organic phase was washed with water and brine, dried and concentrated under vacuum to give methyl 2-acetamido-4-bromo-3-fluoro-5-iodo-benzoate (480.00 g, 95% yield) as a solid. LCMS ESI (+) m/z 416 (M+H). ¹HNMR (400 MHz, CDCl₃) δ 8.88 (s, 1H), 8.25 (s, 1H), 3.94 (s, 3H), 2.25 (s, 3H).

Step D: Preparation of methyl 2-acetamido-4-bromo-3-fluoro-5-(trifluoromethyl)benzoate: To a stirred solution of methyl 2-acetamido-4-bromo-3-fluoro-5-iodo-benzoate (1.00 eq, 150.00 g, 346 mmol) and methyl 2,2-difluoro-2-(fluorosulfonyl)acetate (5.00 eq, 332.32 g, 1731 mmol) in NMP (2 L) at ambient temperature, CuI (2.50 eq, 164.82 g, 865 mmol) was added and the resulting mixture was stirred at 80° C. for 3 hours. Once cooled to ambient temperature, the mixture was quenched with water, filtered, and the filtrate was extracted with ethyl acetate. The organic layer was dried over Na₂SO₄, filtered, concentrated and the crude material was purified by silica gel column chromatography using ethyl acetate in hexanes (0% to 50%) as eluent to give methyl 2-acetamido-4-bromo-3-fluoro-5-(trifluoromethyl)benzoate (100.00 g, 69% yield) as a solid. LCMS ESI (+) m/z 358 (M+H). ¹HNMR (400 MHz, CDCl₃) δ 9.26 (s, 1H), 8.11 (s, 1H), 3.98 (s, 3H), 2.30 (s, 3H).

Step E: Preparation of methyl 2-amino-4-bromo-3-fluoro-5-(trifluoromethyl)benzoate: A mixture of methyl 2-acetamido-4-bromo-3-fluoro-5-(trifluoromethyl)benzoate (1.00 eq, 200.00 g, 559 mmol) in 3M HCl in methanol (2000 mL) was heated at 60° C. for 2 hours. Once cooled to ambient temperature, to the solvent was added saturated NaHCO₃ to pH>7, and the crude product was extracted with ethyl acetate. The organic layer was separated, dried over sodium sulfate, filtered, and evaporated to provide methyl 2-amino-4-bromo-3-fluoro-5-(trifluoromethyl)benzoate (165.00 g, 79% yield) as an oil. LCMS ESI (+) m/z 316 (M+H). ¹HNMR (400 MHz, CDCl₃) δ 8.02 (s, 1H), 6.30 (s, 2H), 3.94 (s, 3H).

Step F: Preparation of methyl 4-bromo-3-fluoro-2-[(2,2,2-trichloroacetyl)carbamoylamino]-5-(trifluoromethyl)benzoate: To a mixture of methyl 2-amino-4-bromo-3-fluoro-5-(trifluoromethyl)benzoate (1.00 eq, 150.00 g, 475 mmol) in THE (2200 mL) was added 2,2,2-trichloroacetyl isocyanate (1.50 eq, 133.13 g, 712 mmol) at ambient temperature. After 15 minutes, the reaction mixture was evaporated to provide methyl 4-bromo-3-fluoro-2-[(2,2,2-trichloroacetyl)carbamoylamino]-5-(trifluoromethyl)benzoate (250.00 g, 421 mmol, 88% yield) as a solid. LCMS ESI (+) m/z 503 (M+H). ¹H NMR (400 MHz, CDCl₃) δ 10.93 (s, 1H), 8.83 (s, 1H), 8.17 (s, 1H), 4.01 (s, 3H).

Step G: Preparation of 7-bromo-8-fluoro-6-(trifluoromethyl)quinazoline-2,4-diol: To a solution of methyl 4-bromo-3-fluoro-2-[(2,2,2-trichloroacetyl)carbamoylamino]-5-(trifluoromethyl)benzoate (1.00 eq, 200.00 g, 396 mmol) in methanol (2000 mL) was added NH₃ in CH₃₀H (7M) (4.00 eq, 200 mL, 1586 mmol) at ambient temperature and stirred at ambient temperature for 1 hour. The mixture was concentrated under reduced pressure to provide a solid. The crude product was purified by silica gel column chromatography using ethyl acetate in hexanes (30% to 100%) as eluent to give 7-bromo-8-fluoro-6-(trifluoromethyl)quinazoline-2,4-diol (90.00 g, 60.39% yield) as a solid. LCMS ESI (+) m/z 327.0 (M+H). ¹H NMR (400 MHz, DMSO-d₆) δ 11.76 (s, 2H), 7.98 (s, 1H).

Step H: Preparation of 7-bromo-2,4-dichloro-8-fluoro-6-(trifluoromethyl)quinazoline: To a stirring solution of phosphorus oxychloride (23.5 eq, 200 mL, 2157 mmol) and DIEA (5.00 eq, 60 g, 459 mmol) was added 7-bromo-8-fluoro-6-(trifluoromethyl)quinazoline-2,4-diol (1.00 eq, 30.00 g, 91.7 mmol) at 0° C. After addition, the resulting mixture was stirred at 110° C. for 1 hour. Once cooled down to ambient temperature, the mixture was evaporated and co-evaporated with chloroform to give 7-bromo-2,4-dichloro-8-fluoro-6-(trifluoromethyl)quinazoline (150.00 g crude, 89% yield) which used as such for the next step. LCMS ESI (+) m/z 363 (M+H).

Step I: Preparation of tert-butyl 3-[7-bromo-2-chloro-8-fluoro-6-(trifluoromethyl)quinazolin-4-yl]-3,8-diazabicyclo[3.2.1]octane-8-carboxylate: To a mixture of 7-bromo-2,4-dichloro-8-fluoro-6-(trifluoromethyl)quinazoline (1.00 eq, 150.00 g crude, 82.4 mmol) in DCM (900 mL) at −40° C. was added TEA (3.00 eq, 25 g, 247 mmol), followed by tert-butyl 3,8-diazabicyclo[3.2.1]octane-8-carboxylate (1.30 eq, 22.75 g, 107 mmol). The reaction mixture was brought slowly to room temperature. The resulting mixture was washed with saturated NaHCO₃ solution. The organic layer was separated and dried with sodium sulfate, filtered, and evaporated. The product was purified by column chromatography using ethyl acetate in hexanes (0% to 100%) as eluent to give tert-butyl 3-[7-bromo-2-chloro-8-fluoro-6-(trifluoromethyl)quinazolin-4-yl]-3,8-diazabicyclo[3.2.1]octane-8-carboxylate (33 g, 70% yield) as a solid. LCMS ESI (+) m/z 539.0 (M+H). ¹H NMR (400 MHz, CDCl₃) δ 7.99 (s, 1H), 4.42-4.34 (m, 4H), 3.78-3.62 (m, 2H), 2.10-1.90 (m, 2H), 1.75-1.65 (m, 2H), 1.54 (s, 9H).

Step J: Preparation of tert-butyl 3-[7-bromo-8-fluoro-2-[[rac-(3R,5R)-3-fluoro-1-methyl-azocan-5-yl]methoxy]-6-(trifluoromethyl)quinazolin-4-yl]-3,8-diazabicyclo[3.2.1]octane-8-carboxylate: To a mixture of tert-butyl 3-[7-bromo-2-chloro-8-fluoro-6-(trifluoromethyl)quinazolin-4-yl]-3,8-diazabicyclo[3.2.1]octane-8-carboxylate (1.00 eq, 8.0 g, 14.8 mmol) and [(2R,8S)-2-fluoro-1,2,3,5,6,7-hexahydropyrrolizin-8-yl]methanol (1.70 eq, 4.01 g, 25.2 mmol) in 1,4-dioxane (80 mL) was added DIEA (5.00 eq, 13 mL, 74.1 mmol) at ambient temperature. The reaction mixture was stirred at 125° C. in stuffy tank. After 48 hours, LCMS showed 70% response. The reaction mixture was cooled to ambient temperature, concentrated under reduced pressure and the residue was purified by flash chromatography on silica gel eluting with 40% EtOAc in hexane to 100% EtOAc to give tert-butyl 3-[7-bromo-8-fluoro-2-[[rac-(3R,5R)-3-fluoro-1-methyl-azocan-5-yl]methoxy]-6-(trifluoromethyl)quinazolin-4-yl]-3,8-diazabicyclo[3.2.1]octane-8-carboxylate (5.00 g, 46% yield) as a solid. LCMS ESI (+) m/z 662.1 (M+H). ¹H NMR (400 MHz, CDCl₃) δ 7.92 (s, 1H), 5.36 (s, 0.5H), 5.23 (s, 0.5H), 4.37-4.27 (m, 4H), 4.26 (d, J=10.4 Hz, 1H), 3.75-3.50 (m, 2H), 3.28-3.25 (m, 2H), 3.18 (d, J=7.2 Hz, 1H), 3.05-2.95 (m, 1H), 2.35-2.15 (m, 3H), 2.00-1.71 (m, 6H), 1.54 (s, 9H).

Step K: Preparation of tert-butyl 3-[7-bromo-2-[[(2R,8S)-2-fluoro-1,2,3,5,6,7-hexahydropyrrolizin-8-yl]methoxy]-6-(trifluoromethyl)-8-(2-trimethylsilylethoxy)quinazolin-4-yl]-3,8-diazabicyclo[3.2.1]octane-8-carboxylate: 2-trimethylsilylethanol (3.00 eq, 134 mg, 1.13 mmol) was added to a stirred solution of THF (1.5 mL) and to this was added NaH (2.00 eq, 30 mg, 0.755 mmol) at ° C. under N₂ and stirred for 30 min, then tert-butyl 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-3,8-diazabicyclo[3.2.1]octane-8-carboxylate (1.00 eq, 250 mg, 0.377 mmol) in THF (2 mL) was added. The resulting solution was stirred at 20° C. for 3 h. The reaction mixture was transferred into water and extracted with ethyl acetate. The organic layer was combined, dried over sodium sulfate, and concentrated under reduced pressure. The crude product was purified by prep-TLC to give tert-butyl 3-[7-bromo-2-[[(2R,8S)-2-fluoro-1,2,3,5,6,7-hexahydropyrrolizin-8-yl]methoxy]-6-(trifluoromethyl)-8-(2-trimethylsilylethoxy)quinazolin-4-yl]-3,8-diazabicyclo[3.2.1]octane-8-carboxylate (45 mg, 14% yield). LCMS ESI (+) m/z 760.1 (M+H).

Step L: Preparation of tert-butyl 3-[7-bromo-2-[[(2R,8S)-2-fluoro-1,2,3,5,6,7-hexahydropyrrolizin-8-yl]methoxy]-8-hydroxy-6-(trifluoromethyl)quinazolin-4-yl]-3,8-diazabicyclo[3.2.1]octane-8-carboxylate: To stirring solution of tert-butyl 3-[7-bromo-2-[[(2R,8S)-2-fluoro-1,2,3,5,6,7-hexahydropyrrolizin-8-yl]methoxy]-6-(trifluoromethyl)-8-(2-trimethylsilylethoxy)quinazolin-4-yl]-3,8-diazabicyclo[3.2.1]octane-8-carboxylate (1.00 eq, 20 mg, 0.0263 mmol) in DMF (2 mL) was added CsF (3.00 eq, 12 mg, 0.0789 mmol) at 20° C. The resulting mixture was heated to 60° C. for 2 hours. The solvent was dissolved in water and ethyl acetate. The organic layer was concentrated under vacuum. The crude product was purified by prep-TLC to give tert-butyl 3-[7-bromo-2-[[(2R,8S)-2-fluoro-1,2,3,5,6,7-hexahydropyrrolizin-8-yl]methoxy]-8-hydroxy-6-(trifluoromethyl)quinazolin-4-yl]-3,8-diazabicyclo[3.2.1]octane-8-carboxylate (8.0 mg, 43% yield). LCMS ESI (+) m/z 661.2 (M+H).

Step M: Preparation of tert-butyl 3-[7-[2-(tert-butoxycarbonylamino)-7-fluoro-1,3-benzothiazol-4-yl]-2-[[(2R,8S)-2-fluoro-1,2,3,5,6,7-hexahydropyrrolizin-8-yl]methoxy]-8-hydroxy-6-(trifluoromethyl)quinazolin-4-yl]-3,8-diazabicyclo[3.2.1]octane-8-carboxylate: To a stirring suspension of tert-butyl 3-[7-bromo-2-[[(2R,8S)-2-fluoro-1,2,3,5,6,7-hexahydropyrrolizin-8-yl]methoxy]-8-hydroxy-6-(trifluoromethyl)quinazolin-4-yl]-3,8-diazabicyclo[3.2.1]octane-8-carboxylate (1.00 eq, 8.0 mg, 0.0121 mmol) in 1,4-dioxane (1 mL), [2-(tert-butoxycarbonylamino)-7-fluoro-1,3-benzothiazol-4-yl]boronic acid (2.00 eq, 7.6 mg, 0.0242 mmol) and K₃PO₄ (2.00 eq, 5.1 mg, 0.0242 mmol) were added, followed by water (0.25 mL) at ambient temperature. Then SPhos Pd G2 (0.20 eq, 1.7 mg, 0.0024 mmol) was added, the reaction mixture was degassed with N₂. The reaction mixture was stirred at 90° C. for 90 minutes. LCMS showed the products. The solvent was dissolved in water and ethyl acetate. The organic layer was concentrated under vacuum. The crude product was purified by prep-TLC to give tert-butyl 3-[7-[2-(tert-butoxycarbonylamino)-7-fluoro-1,3-benzothiazol-4-yl]-2-[[(2R,8S)-2-fluoro-1,2,3,5,6,7-hexahydropyrrolizin-8-yl]methoxy]-8-hydroxy-6-(trifluoromethyl)quinazolin-4-yl]-3,8-diazabicyclo[3.2.1]octane-8-carboxylate (4.0 mg, 37% yield). LCMS ESI (+) m/z 848.2 (M+H).

Step N: Preparation of 7-(2-amino-7-fluoro-1,3-benzothiazol-4-yl)-4-(3,8-diazabicyclo[3.2.1]octan-3-yl)-2-[[(2R,8S)-2-fluoro-1,2,3,5,6,7-hexahydropyrrolizin-8-yl]methoxy]-6-(trifluoromethyl)quinazolin-8-ol: To a solution of tert-butyl 3-[7-[2-(tert-butoxycarbonylamino)-7-fluoro-1,3-benzothiazol-4-yl]-2-[[(2R,8S)-2-fluoro-1,2,3,5,6,7-hexahydropyrrolizin-8-yl]methoxy]-8-hydroxy-6-(trifluoromethyl)quin azolin-4-yl]-3,8-diazabicyclo[3.2.1]octane-8-carboxylate (1.00 eq, 4.0 mg, 0.00472 mmol) in DCM (2 mL) was added excess TFA (1707 eq, 0.60 mL, 8.05 mmol) and the reaction was stirred for 12 hours at room temperature. The solvent was concentrated under reduced pressure and lyophilized with water to obtain the 7-(2-amino-7-fluoro-1,3-benzothiazol-4-yl)-4-(3,8-diazabicyclo[3.2.1]octan-3-yl)-2-[[(2R,8S)-2-fluoro-1,2,3,5,6,7-hexahydropyrrolizin-8-yl]methoxy]-6-(trifluoromethyl)quinazolin-8-ol trifluoroacetate (3.0 mg, 90% yield). LCMS ESI(+) m/z 648 (M+1). ¹H NMR (400 MHz, CD₃OD) δ 8.12 (s, 1H), 7.28-7.26 (m, 1H), 7.08-7.06 (m, 1H), 5.38-5.36 (m, 1H), 5.25 (s, 0.5H), 5.11 (s, 0.5H), 4.31-5.28 (m, 3H), 4.04-3.81 (m, 6H), 2.22-1.92 (m, 8H), 1.84-1.79 (m, 2H), 1.66-1.60 (m, 2H).

### Synthetic Example 43: Synthesis of 7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-4-(methyl((R)-pyrrolidin-3-yl)amino)quinazoline-6-carbonitrile (Compound 345)

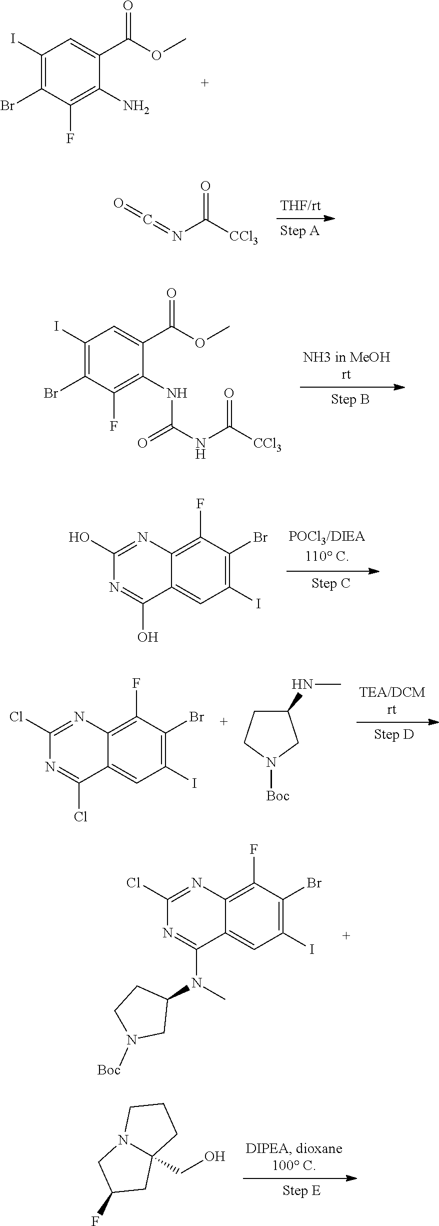

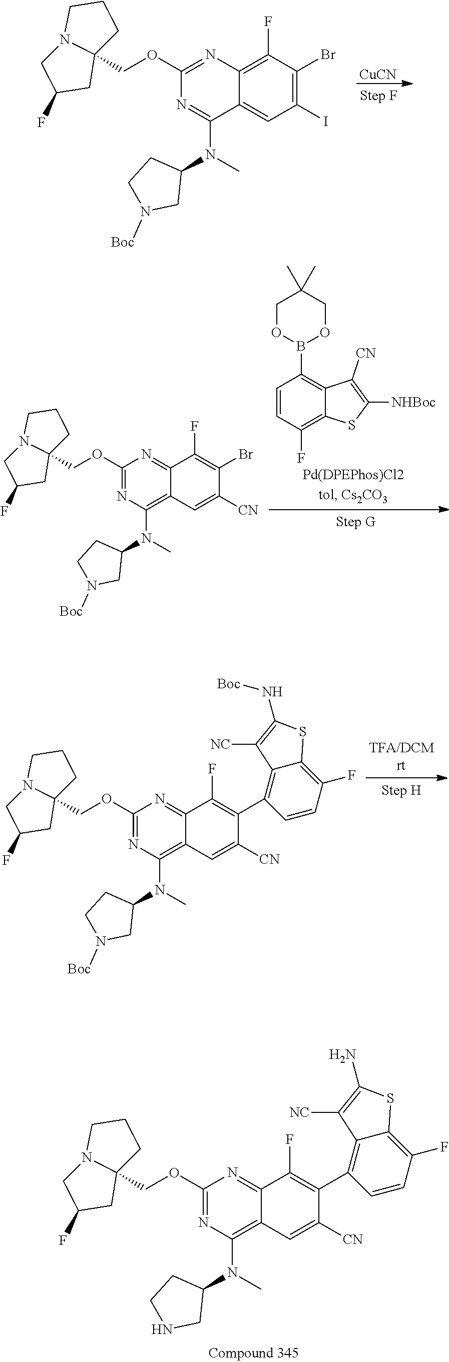

Step A: Preparation of methyl 4-bromo-3-fluoro-5-iodo-2-[(2,2,2-trichloroacetyl)carbamoylamino]benzoate: To solution of methyl 2-amino-4-bromo-3-fluoro-5-iodo-benzoate (1.00 eq, 10.00 g, 26.7 mmol) in THE (100 mL) was added 2,2,2-trichloroacetyl isocyanate (1.50 eq, 7557 mg, 40.1 mmol). After addition the reaction mixture was stirred 25° C. for 1 h. The solvent was removed, and the residue was triturated with MTBE to give methyl 4-bromo-3-fluoro-5-iodo-2-[(2,2,2-trichloroacetyl)carbamoylamino]benzoate (13.00 g, 20.8 mmol, 77% yield) as a solid. LCMS ESI (+) m/z 563.7 (M+H), 585.6 (M+23). ¹H NMR (400 MHz, DMSO-d6) δ 11.83 (brs, 1H), 10.04 (s, 1H), 8.17 (s, 1H), 3.82 (s, 3H).

Step B: Preparation of 7-bromo-8-fluoro-6-iodo-quinazoline-2,4-diol: To a solution of methyl 4-bromo-3-fluoro-5-iodo-2-[(2,2,2-trichloroacetyl)carbamoylamino]benzoate (1.00 eq, 13.00 g, 23.1 mmol) in methanol (50 mL) was added NH₃ in CH₃OH (7M) (1.00 eq, 80 mL, 23.1 mmol) at ambient temperature and stirred at ambient temperature for 1 hour. The mixture was concentrated under reduced pressure, and the residue was triturated with ether to give 7-bromo-8-fluoro-6-iodo-quinazoline-2,4-diol (9.70 g, 98% yield) as a solid. LCMS ESI (+) m/z 384.9 (M+H).

Step C: Preparation of 7-bromo-2,4-dichloro-8-fluoro-6-iodo-quinazoline: To a stirring solution of phosphorus oxychloride (23.0 eq, 27426 mg, 179 mmol) and DIPEA (5.00 eq, 5036 mg, 39.0 mmol) was added 7-bromo-8-fluoro-6-iodo-quinazoline-2,4-diol (1.00 eq, 3.00 g, 7.79 mmol) at 0° C. After addition, the resulting mixture was stirred at 110° C. for 4 hours. Once cooled down to ambient temperature, the mixture was evaporated and co-evaporated with chloroform to give 7-bromo-2,4-dichloro-8-fluoro-6-iodo-quinazoline (7.00 g, 95% yield) which used directly in next step without further purification. LCMS ESI (+) m/z 421.8 (M+H).

Step D: Preparation of tert-butyl (3S)-3-[(7-bromo-2-chloro-8-fluoro-6-iodo-quinazolin-4-yl)-methyl-amino]pyrrolidine-1-carboxylate: To a solution of 7-bromo-2,4-dichloro-8-fluoro-6-iodo-quinazoline (1.00 eq, 4.00 g, 1.90 mmol) in DCM (10 mL) was added TEA (3.00 eq, 0.79 mL, 5.69 mmol), then tert-butyl (3R)-3-(methylamino)pyrrolidine-1-carboxylate (1.30 eq, 0.49 g, 2.47 mmol) was added at −40° C., after addition the reaction mixture was stirred at rt overnight. The reaction mixture was washed with water and brine, dried over Na₂SO₄, concentrated. The residue was purified by passing a silica gel column (PE/EA=2/1) to give tert-butyl (3S)-3-[(7-bromo-2-chloro-8-fluoro-6-iodo-quinazolin-4-yl)-methyl-amino]pyrrolidine-1-carboxylate (310 mg, 20% yield) as a solid. LCMS ESI (+) m/z 586 (M+H).

Step E: Preparation of tert-butyl (3S)-3-[[7-bromo-8-fluoro-2-[[(2R,8S)-2-fluoro-1,2,3,5,6,7-hexahydropyrrolizin-8-yl]methoxy]-6-iodo-quinazolin-4-yl]-methyl-amino]pyrrolidine-1-carboxylate: To a solution of tert-butyl (3S)-3-[(7-bromo-2-chloro-8-fluoro-6-iodo-quinazolin-4-yl)-methyl-amino]pyrrolidine-1-carboxylate (1.00 eq, 430 mg, 0.734 mmol) in 1,4-dioxane (1 mL) was added [(2R,8S)-2-fluoro-1,2,3,5,6,7-hexahydropyrrolizin-8-yl]methanol (2.00 eq, 234 mg, 1.47 mmol) and DIPEA (5.00 eq, 0.64 mL, 3.67 mmol), after addition the reaction mixture was stirred at 100° C. for 48 hours. The solvent was removed, and the residue was dissolved in ethyl acetate (50 mL), washed with water and brine, dried over Na₂SO₄, and concentrated. The residue was purified by prep-TLC to give tert-butyl (3S)-3-[[7-bromo-8-fluoro-2-[[(2R,8S)-2-fluoro-1,2,3,5,6,7-hexahydropyrrolizin-8-yl]methoxy]-6-iodo-quinazolin-4-yl]-methyl-amino]pyrrolidine-1-carboxylate (150 mg, 25% yield) as a solid. LCMS ESI (+) m/z 708 (M+H).

Step F: Preparation of tert-butyl (3R)-3-[[7-bromo-6-cyano-8-fluoro-2-[[(2R,8S)-2-fluoro-1,2,3,5,6,7-hexahydropyrrolizin-8-yl]methoxy]quinazolin-4-yl]-methyl-amino]pyrrolidine-1-carboxylate: To a solution of tert-butyl (3S)-3-[[7-bromo-8-fluoro-2-[[(2R,8S)-2-fluoro-1,2,3,5,6,7-hexahydropyrrolizin-8-yl]methoxy]-6-iodo-quinazolin-4-yl]-methyl-amino]pyrrolidine-1-carboxylate (1.00 eq, 140 mg, 0.198 mmol) in DMSO (0.5 mL) was added CuCN (3.00 eq, 53 mg, 0.59 mmol), after addition the reaction mixture was stirred at 80° C. overnight. The reaction mixture was poured into ice water, extracted with ethyl acetate (3×10 mL), the combined organic phase was washed with water and brine, dried over Na₂SO₄, concentrated. The residue was purified with prep-TLC to give tert-butyl (3R)-3-[[7-bromo-6-cyano-8-fluoro-2-[[(2R,8S)-2-fluoro-1,2,3,5,6,7-hexahydropyrrolizin-8-yl]methoxy]quinazolin-4-yl]-methyl-amino]pyrrolidine-1-carboxylate (70 mg, 55% yield) as a solid. LCMS ESI (+) m/z 607.3 (M+H).

Step G: Preparation of tert-butyl (3R)-3-[[7-[2-(tert-butoxycarbonylamino)-3-cyano-7-fluoro-benzothiophen-4-yl]-6-cyano-8-fluoro-2-[[(2R,8S)-2-fluoro-1,2,3,5,6,7-hexahydropyrrolizin-8-yl]methoxy]quinazolin-4-yl]-methyl-amino]pyrrolidine-1-carboxylate: To a solution of tert-butyl (3R)-3-[[7-bromo-6-cyano-8-fluoro-2-[[(2R,8S)-2-fluoro-1,2,3,5,6,7-hexahydropyrrolizin-8-yl]methoxy]quinazolin-4-yl]-methyl-amino]pyrrolidine-1-carboxylate (1.00 eq, 75 mg, 0.123 mmol) in toluene (0.5 mL) was added tert-butyl N-[3-cyano-4-(5,5-dimethyl-1,3,2-dioxaborinan-2-yl)-7-fluoro-benzothiophen-2-yl]carbamate (1.50 eq, 75 mg, 0.185 mmol), Cs₂CO₃ (2.50 eq, 101 mg, 0.309 mmol) and DPEPhosPdCl₂ (0.100 eq, 8.8 mg, 0.0123 mmol), after addition the reaction mixture was stirred at 90° C. for 4 hours. The solvent was removed, and the residue was dissolved in ethyl acetate (50 mL), washed with water (10 mL) and brine (10 mL), dried over Na₂SO₄, filtered and concentrated. The residue was purified by preparative RP-HPLC to give tert-butyl (3R)-3-[[7-[2-(tert-butoxycarbonylamino)-3-cyano-7-fluoro-benzothiophen-4-yl]-6-cyano-8-fluoro-2-[[(2R,8S)-2-fluoro-1,2,3,5,6,7-hexahydropyrrolizin-8-yl]methoxy]quinazolin-4-yl]-methyl-amino]pyrrolidine-1-carboxylate (7.0 mg, 6% yield) as a solid. LCMS ESI (+) m/z 819.3 (M+H).

Step H: Preparation of 7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-4-[methyl-[(3R)-pyrrolidin-3-yl]amino]quinazoline-6-carbonitrile: To a solution of tert-butyl (3R)-3-[[7-[2-(tert-butoxycarbonylamino)-3-cyano-7-fluoro-benzothiophen-4-yl]-6-cyano-8-fluoro-2-[[(2R,8S)-2-fluoro-1,2,3,5,6,7-hexahydropyrrolizin-8-yl]methoxy]quinazolin-4-yl]-methyl-amino]pyrrolidine-1-carboxylate (1.00 eq, 4.0 mg, 0.00488 mmol) in DCM (3 mL) was added TFA (1.0 mL, 13.4 mmol), after addition the reaction mixture was stirred at 25° C. overnight. The solvent was removed, and the residue was lyophilized under reduced pressure to give 7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-4-[methyl-[(3R)-pyrrolidin-3-yl]amino]quinazoline-6-carbonitrile (2.0 mg, 48% yield) as a solid. LCMS ESI (+) m/z 619.0 (M+H). ¹H NMR (400 MHz, CD₃OD) δ 8.52 (s, 1H), 7.38-7.35 (m, 1H), 7.12 (t, J=8.8 Hz, 1H), 5.64 (s, 0.5H), 5.51 (s, 0.5H), 5.39-5.34 (m, 2H), 4.81-4.64 (m, 2H), 3.95-3.86 (m, 3H), 3.70-3.67 (m, 1H), 3.55 (s, 3H), 3.51-3.43 (m, 3H), 2.65-2.56 (m, 2H), 2.45-2.31 (m, 1H), 2.40-2.30 (m, 2H), 2.04 (t, 1H), 2.08-2.01 (m, 1H), 1.62 (t, 1H), 1.40-1.29 (m, 1H).

### Synthetic Example 44: Synthesis of 7-(2-amino-3-cyano-7-fluorobenzo[b]thiophen-4-yl)-4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazoline-6-carbonitrile (Compound 359)

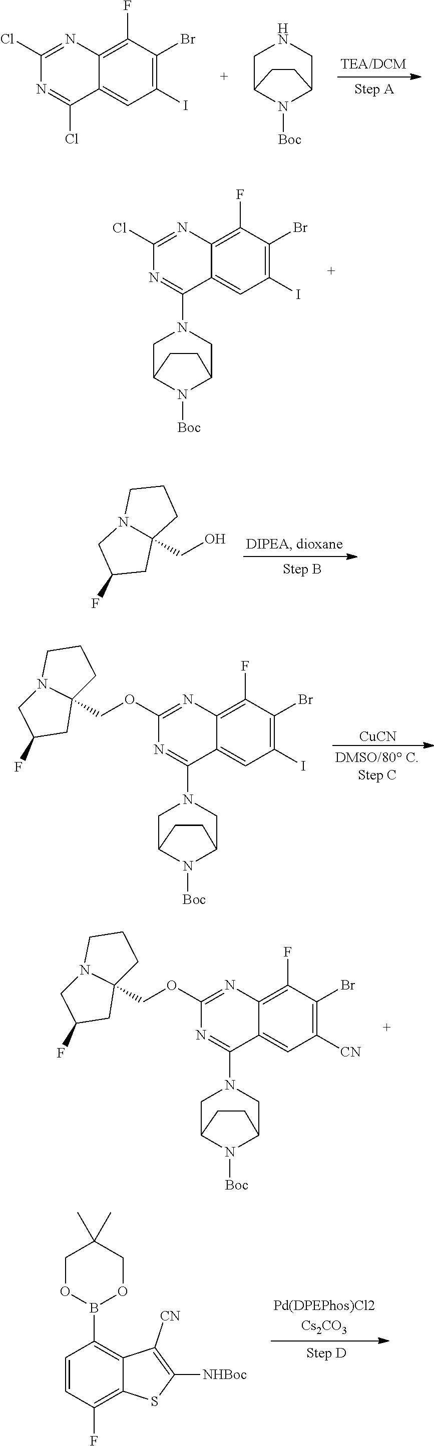

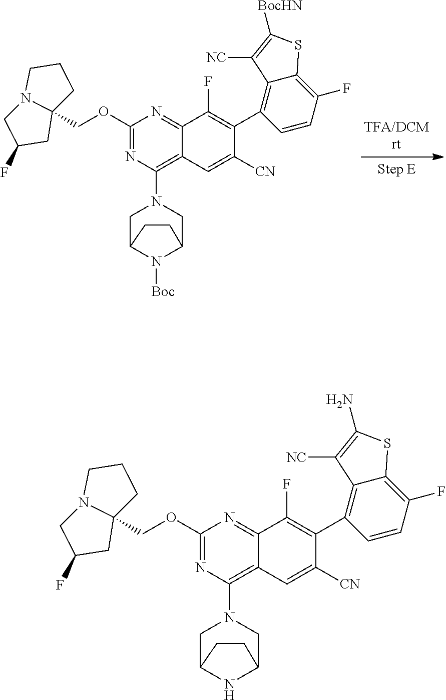 [SMILES: CC(C)(C)OC(=O)Nc1sc2c(F)ccc(-c3c(C#N)cc4c(N5CC6CCC(C5)N6C(=O)OC(C)(C)C)nc(OC[C@@]56CCCN5C[C@H](F)C6)nc4c3F)c2c1C#N ; N#Cc1cc2c(N3CC4CCC(C3)N4)nc(OC[C@@]34CCCN3C[C@H](F)C4)nc2c(F)c1-c1ccc(F)c2sc(N)c(C#N)c12]

Step A: Preparation of tert-butyl (1R,5S)-3-(7-bromo-2-chloro-8-fluoro-6-iodoquinazolin-4-yl)-3,8-diazabicyclo[3.2.1]octane-8-carboxylate: To a mixture of 7-bromo-2,4-dichloro-8-fluoro-6-iodo-quinazoline (1.00 eq, 20.00 g (crude), 9.48 mmol) in DCM (100 mL) at −40° C. was added TEA (3.00 eq, 4.0 mL, 28.4 mmol), followed by tert-butyl 3,8-diazabicyclo[3.2.1]octane-8-carboxylate (1.30 eq, 2.62 g, 12.3 mmol). The reaction mixture was brought slowly to room temperature. The resulting mixture was quenched with EtOAc and saturated NaHCO₃ solution. The organic layer was separated and dried with sodium sulfate, filtered, and evaporated. The crude mixture was purified with flash chromatography on silica gel eluting with 5% EtOAc in PE to 40% EtOAc in PE to provide tert-butyl 3-(7-bromo-2-chloro-8-fluoro-6-iodo-quinazolin-4-yl)-3,8-diazabicyclo[3.2.1]octane-8-carboxylate (3.40 g, 57% yield). LCMS ESI (+) m/z 597.1 (M+H).

Step B: Preparation of tert-butyl 3-[7-bromo-8-fluoro-2-[[(2R,8S)-2-fluoro-1,2,3,5,6,7-hexahydropyrrolizin-8-yl]methoxy]-6-iodo-quinazolin-4-yl]-3,8-diazabicyclo[3.2.1]octane-8-carboxylate: To a mixture of tert-butyl 3-(7-bromo-2-chloro-8-fluoro-6-iodo-quinazolin-4-yl)-3,8-diazabicyclo[3.2.1]octane-8-carboxylate (1.00 eq, 1.00 g, 1.67 mmol) and [(2R,8S)-2-fluoro-1,2,3,5,6,7-hexahydropyrrolizin-8-yl]methanol (1.50 eq, 0.40 g, 2.51 mmol) in 1,4-dioxane (20 mL) was added DIPEA (5.00 eq, 1.5 mL, 8.37 mmol) at ambient temperature. The reaction mixture was stirred at 110° C. After 48 hours, the reaction mixture was cooled to room temperature, diluted with water, and extracted with EtOAc. The organic layer was collected and dried over sodium sulfate, filtered, and evaporated. The crude product was purified by prep-HPLC to obtain tert-butyl 3-[7-bromo-8-fluoro-2-[[(2R,8S)-2-fluoro-1,2,3,5,6,7-hexahydropyrrolizin-8-yl]methoxy]-6-iodo-quinazolin-4-yl]-3,8-diazabicyclo[3.2.1]octane-8-carboxylate (450 mg, 33% yield). LCMS ESI (+) m/z 719.9 (M+H).

Step C: Preparation of tert-butyl 3-[7-bromo-6-cyano-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate: To a solution of tert-butyl 3-[7-bromo-8-fluoro-2-[[(2R,8S)-2-fluoro-1,2,3,5,6,7-hexahydropyrrolizin-8-yl]methoxy]-6-iodo-quinazolin-4-yl]-3,8-diazabicyclo[3.2.1]octane-8-carboxylate (1.00 eq, 400 mg, 0.555 mmol) in DMSO (20 mL) was added CuCN (3.00 eq, 148 mg, 1.67 mmol), after addition the reaction mixture was stirred at 80° C. for 14 h. The mixture was poured into water and filtered. The filtrate was extracted with EtOAc, the organic layer was dried over Na₂SO₄, filtered and concentrated in vacuo, the residue was purified by column chromatography with 5% MeOH in DCM to 20% MeOH in DCM to give tert-butyl 3-[7-bromo-6-cyano-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (210 mg, 30% yield). LCMS ESI (+) m/z 619.0 (M+H).

Step D: Preparation of tert-butyl (1R,5S)-3-(7-(2-((tert-butoxycarbonyl)amino)-3-cyano-7-fluorobenzo[b]thiophen-4-yl)-6-cyano-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate: To a stirring suspension of tert-butyl 3-[7-bromo-6-cyano-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (1.00 eq, 180 mg, 0.291 mmol) in THE (12 mL), Cs₂CO₃ (3.00 eq, 284 mg, 0.872 mmol) and tert-butyl N-[3-cyano-4-(5,5-dimethyl-1,3,2-dioxaborinan-2-yl)-7-fluoro-benzothiophen-2-yl] carbamate (2.00 eq, 235 mg, 0.58 mmol) were added, followed by bis(diphenylphosphinophenyl)ether palladium(II) dichloride (0.150 eq, 31 mg, 0.0436 mmol) and the mixture was degassed again for 1 minute. The reaction mixture was stirred at 70° C. for 12 h. LCMS showed the products. The reaction mixture was cooled to room temperature, diluted with water, and extracted with EtOAc. The organic layer was collected and dried over sodium sulfate, filtered, and evaporated. The crude product was purified by prep-TLC to obtain the crude product. Then it was purified under preparative RP-HPLC to give tert-butyl (1R,5S)-3-(7-(2-((tert-butoxycarbonyl)amino)-3-cyano-7-fluorobenzo[b]thiophen-4-yl)-6-cyano-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (35 mg, 14% yield). LCMS ESI (+) m/z 831.1 (M+H).

Step E: Preparation of 7-(2-amino-3-cyano-7-fluoro-benzothiophen-4-yl)-4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]quinazoline-6-carbonitrile: To a solution of tert-butyl 3-[7-[2-(tert-butoxycarbonylamino)-3-cyano-7-fluoro-benzothiophen-4-yl]-6-cyano-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (1.00 eq, 35 mg, 0.0421 mmol) in DCM (9 mL) was added excess TFA (956 eq, 3.0 mL, 40.3 mmol) and the reaction was stirred for 12 hours at room temperature. The solvent was concentrated under reduced pressure and lyophilized with water. The crude product was purified by Preparative RP-HPLC to give 7-(2-amino-3-cyano-7-fluoro-benzothiophen-4-yl)-4-(3,8-diazabicyclo [3.2.1]octan-3-yl)-8-fluoro-2-[[(2R,8S)-2-fluoro-1,2,3,5,6,7-hexahydropyrrolizin-8-yl]methoxy] quinazoline-6-carbonitrile (22 mg, 81% yield). LCMS ESI (+) m/z 631.0 (M+H). ¹HNMR (400 MHz, CD₃OD) δ 8.36 (s, 1H), 7.37-7.33 (m, 1H), 7.12 (t, J=8.8 Hz, 1H), 5.65 (s, 0.5H), 5.52 (s, 0.5H), 4.87-4.64 (m, 4H), 4.26 (s, 2H), 4.09-3.94 (m, 5H), 3.53-3.46 (m, 1H), 2.77-2.57 (m, 3H), 2.50-2.32 (m, 3H), 2.22-2.07 (m, 4H).

### Synthetic Example 45: Synthesis of 2-amino-4-(6,8-difluoro-2-(((2R,7aS)-2-fluorotetrahydro-1H-pyrrolizin-7a(5H)-yl)methoxy)-4-(methyl((S)-pyrrolidin-3-yl)amino)quinazolin-7-yl)-7-fluorobenzo[b]thiophene-3-carbonitrile (Compound 361)

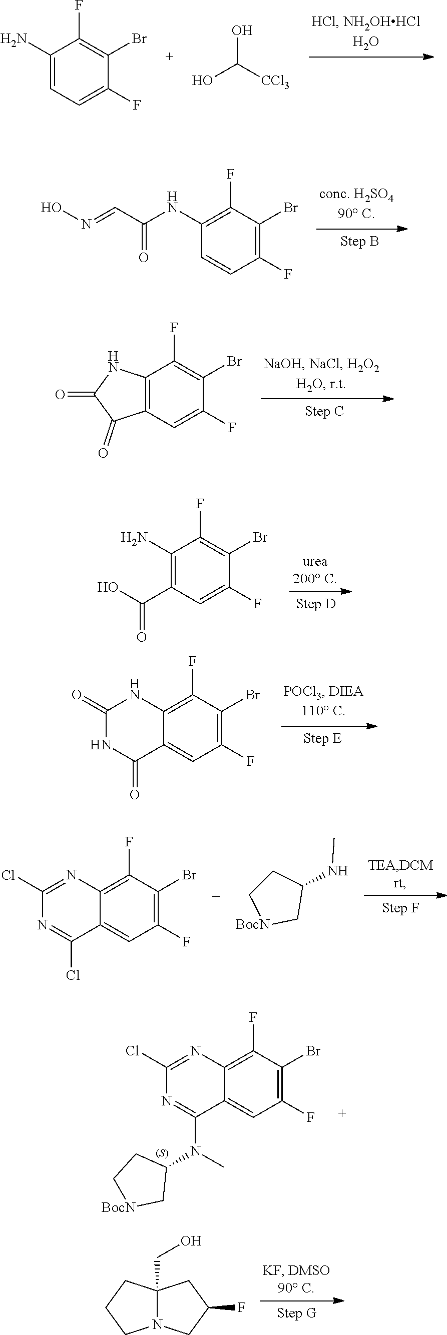

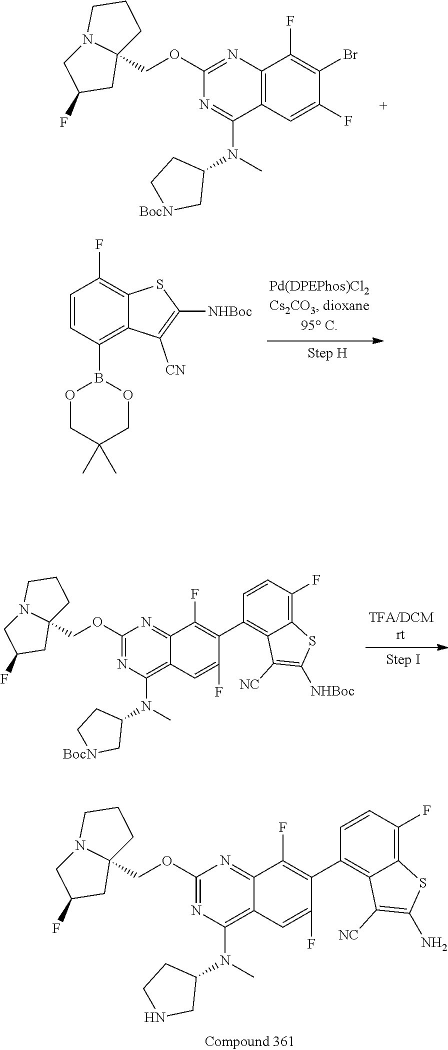 [SMILES: CN(c1nc(OC[C@@]23CCCN2C[C@H](F)C3)nc2c(F)c(Br)c(F)cc12)[C@H]1CCN(C(=O)OC(C)(C)C)C1 ; CC1(C)COB(c2ccc(F)c3sc(NC(=O)OC(C)(C)C)c(C#N)c23)OC1 ; CN(c1nc(OC[C@@]23CCCN2C[C@H](F)C3)nc2c(F)c(-c3ccc(F)c4sc(NC(=O)OC(C)(C)C)c(C#N)c34)c(F)cc12)[C@H]1CCN(C(=O)OC(C)(C)C)C1 ; CN(c1nc(OC[C@@]23CCCN2C[C@H](F)C3)nc2c(F)c(-c3ccc(F)c4sc(N)c(C#N)c34)c(F)cc12)[C@H]1CCNC1]

Step A: Preparation of (E)-N-(3-bromo-2,4-difluorophenyl)-2-(hydroxyimino)acetamide: To a solution of 3-bromo-2,4-difluoro-aniline (25.0 g, 120 mmol) in hydrochloric acid (17 mL, 566 mmol) and water (200 mL) was added a solution of sodium sulfate (160.5 g, 1130 mmol) and 2,2,2-trichloroethane-1,1-diol (25.8 g, 156 mmol) in water (500 mL) at ambient temperature. Then, hydroxylamine hydrochloride (30.1 g, 433 mmol) in water (150 mL) was added at ambient temperature. The resulting solution was stirred at 100° C. for 2 hours. After cooled to rt, the solid was collected by filtration and washed with water. The solid was dissolved in EtOAc and washed with brine. The organic phase was dried over anhydrous sodium sulfate, filtered and concentrated under reduced pressure to give (2E)-N-(3-bromo-2,4-difluoro-phenyl)-2-hydroxyimino-acetamide (31.00 g, 91%).

Step B: Preparation of 6-bromo-5,7-difluoroindoline-2,3-dione: (2E)-N-(3-bromo-2,4-difluoro-phenyl)-2-hydroxyimino-acetamide (1.00 eq, 28.00 g, 100 mmol) was added portion wise to conc. sulfuric acid (300 mL). After addition, the mixture was stirred at 90° C. for 2 hours. The reaction mixture was cooled to room temperature and slowly added to ice water. The resulting precipitate was collected by filtration, washing with water and dried under vacuum to afford 6-bromo-5,7-difluoro-indoline-2,3-dione (27.0 g).

Step C: Preparation of 2-amino-4-bromo-3,5-difluorobenzoic acid: The solution of 6-bromo-5,7-difluoro-indoline-2,3-dione (1.00 eq, 820 mg, 3.13 mmol), sodium chloride (2.40 eq, 440 mg, 7.53 mmol) and sodium hydroxide (2.10 eq, 260 mg, 6.50 mmol) in water (20 mL) was stirred at rt for 0.5 h. Then, hydrogen peroxide (1.00 eq, 1.0 mL, 32.6 mmol) was added slowly, followed by NaOH solution (0.6 g NaOH in 20 mL of water). The reaction mixture was stirred at rt for 5 hours. The mixture was extracted with ethyl ether. The aqueous solution was collected, acidified with 3N HCl to pH=3-5, and then it was extracted with DCM, dried over anhydrous sodium sulfate, filtered and concentrated to dryness to give 2-amino-4-bromo-3,5-difluoro-benzoic acid (306 mg, 38%).

Step D: Preparation of 7-bromo-6,8-difluoroquinazoline-2,4(1H,3H)-dione: 2-amino-4-bromo-3,5-difluoro-benzoic acid (1.00 eq, 6.90 g, 27.4 mmol) and urea (20.0 eq, 32.89 g, 548 mmol) were placed in 250 mL round bottom flask and heated to 200° C. after mixed well. The mixture was melted and then solidified again. After 2 hours at 200° C., the mixture was cooled to 100° C. and then to this was added 400 mL water and stirred for 2 hours at 100° C. The solid was evenly dispersed. After cooled to room temperature, the solid was collected by filtration, washed with small amount of ACN and dried under vacuum to give 7-bromo-6,8-difluoro-1H-quinazoline-2,4-dione (4.30 g, 56%).

Step E: Preparation of 7-bromo-2,4-dichloro-6,8-difluoroquinazoline: A solution of 7-bromo-6,8-difluoro-1H-quinazoline-2,4-dione (1.0 g, 3.61 mmol) and DIEA (3.3 mL, 18 mmol) in POCl₃ (6 mL) was stirred at 110° C. for 2 hours. After cooled to ambient temperature, the mixture was concentrated in vacuo and purified by column chromatography on silica gel to afford 7-bromo-2,4-dichloro-6,8-difluoroquinazoline (313 mg, 28%).

Step F: Preparation of tert-butyl (S)-3-((7-bromo-2-chloro-6,8-difluoroquinazolin-4-yl)(methyl)amino)pyrrolidine-1-carboxylate: A solution of 7-bromo-2,4-dichloro-6,8-difluoroquinazoline (313 mg, 1.0 mmol) and DIEA (0.55 mL, 3 mmol) in DCM (5 mL) was added tert-butyl (S)-3-(methylamino)pyrrolidine-1-carboxylate (160 mg, 0.8 mmol) at −40° C. and stirred at this temperature for 1 hour. The reaction mixture was quenched with water and extracted with DCM, the organic layer was washed with water and brine, dried over sodium sulfate, filtered and concentrated under reduced pressure. The residue was purified by column chromatography on silica gel to afford tert-butyl (S)-3-((7-bromo-2-chloro-6,8-difluoroquinazolin-4-yl)(methyl)amino)pyrrolidine-1-carboxylate (170 mg, 0.36 mmol, 36% yield). LCMS ESI (+) m/z 477.1 (M+H).

Step G: Preparation of tert-butyl (S)-3-((7-bromo-6,8-difluoro-2-(((2R,7aS)-2-fluorotetrahydro-1H-pyrrolizin-7a(5H)-yl)methoxy)quinazolin-4-yl)(methyl)amino)pyrrolidine-1-carboxylate: A solution of tert-butyl (S)-3-((7-bromo-2-chloro-6,8-difluoroquinazolin-4-yl)(methyl)amino)pyrrolidine-1-carboxylate (170 mg, 0.36 mmol) and KF (167 mg, 2.88 mmol) in DMSO (2 mL) was stirred at 100° C. for 6 hours, then ((2R,7aS)-2-fluorotetrahydro-1H-pyrrolizin-7a(5H)-yl)methanol (114 mg, 0.72 mmol) was added. The mixture was stirred at 110° C. for another 24 hours. After cooled to ambient temperature, the reaction mixture was quenched with water and extracted with EtOAc, the organic layer washed with water and brine, dried over sodium sulfate, filtered and concentrated under reduced pressure. The residue was purified by Preparative-TLC to give tert-butyl (S)-3-((7-bromo-6,8-difluoro-2-(((2R,7aS)-2-fluorotetrahydro-1H-pyrrolizin-7a(5H)-yl)methoxy)quinazolin-4-yl)(methyl)amino)pyrrolidine-1-carboxylate (60 mg, 28%). LCMS (ES+) m/z 600.3 (M+H).

Step H: Preparation of tert-butyl (3S)-3-((7-(2-((tert-butoxycarbonyl)amino)-3-cyano-7-fluorobenzo[b]thiophen-4-yl)-6,8-difluoro-2-(((2R,7aS)-2-fluorotetrahydro-1H-pyrrolizin-7a(5H)-yl)methoxy)quinazolin-4-yl)(methyl)amino)pyrrolidine-1-carboxylate: A solution of tert-butyl (S)-3-((7-bromo-6,8-difluoro-2-(((2R,7aS)-2-fluorotetrahydro-1H-pyrrolizin-7a(5H)-yl)methoxy)quinazolin-4-yl)(methyl)amino)pyrrolidine-1-carboxylate (60 mg, 0.10 mmol), tert-butyl (3-cyano-4-(5,5-dimethyl-1,3,2-dioxaborinan-2-yl)-7-fluorobenzo[b]thiophen-2-yl)carbamate (80.8 mg, 0.2 mmol), Cs₂CO₃ (65.2 mg, 0.2 mmol) and Pd(DPEPhos)Cl₂ (14.3 mg, 0.02 mmol) in dioxane (1.5 mL) was stirred at 100° C. for 4 hours. After cooled to ambient temperature, the reaction was quenched with water and extracted with EtOAc, the organic layer was washed with water and brine, dried over sodium sulfate, filtered and the solvent was removed by rotary evaporation. The residue was purified by flash chromatography on silica gel to give tert-butyl (3S)-3-((7-(2-((tert-butoxycarbonyl)amino)-3-cyano-7-fluorobenzo[b]thiophen-4-yl)-6,8-difluoro-2-(((2R,7aS)-2-fluorotetrahydro-1H-pyrrolizin-7a(5H)-yl)methoxy)quinazolin-4-yl)(methyl)amino)pyrrolidine-1-carboxylate (9 mg, 11%). LCMS (ES+) m/z 812.3 (M+H).

Step I: Preparation of 2-amino-4-(6,8-difluoro-2-(((2R,7aS)-2-fluorotetrahydro-1H-pyrrolizin-7a(5H)-yl)methoxy)-4-(methyl((S)-pyrrolidin-3-yl)amino)quinazolin-7-yl)-7-fluorobenzo[b]thiophene-3-carbonitrile: A solution of tert-butyl (3S)-3-((7-(2-((tert-butoxycarbonyl)amino)-3-cyano-7-fluorobenzo[b]thiophen-4-yl)-6,8-difluoro-2-(((2R,7aS)-2-fluorotetrahydro-1H-pyrrolizin-7a(5H)-yl)methoxy)quinazolin-4-yl)(methyl)amino)pyrrolidine-1-carboxylate (9 mg, 0.011 mmol) in DCM/TFA (2 mL/1 mL) was stirred at room temperature for 2 hours. The solvent was removed by rotary evaporation and purified by Preparative RP-HPLC to give 2-amino-4-(6,8-difluoro-2-(((2R,7aS)-2-fluorotetrahydro-1H-pyrrolizin-7a(5H)-yl)methoxy)-4-(methyl((S)-pyrrolidin-3-yl)amino)quinazolin-7-yl)-7-fluorobenzo[b]thiophene-3-carbonitrile (3.0 mg, 45%). LCMS ESI (+) m/z 612.4 (M+H). ¹HNMR (400 MHz, CD₃OD) δ 7.82 (d, J=10.0 Hz, 1H), 7.28-7.32 (m, 1H), 7.06 (t, J=8.94 Hz, 1H), 5.53 (d, J=51.2 Hz, 1H), 5.19-5.23 (m, 1H), 4.64-4.78 (m, 2H), 3.79-3.96 (m, 4H), 3.67-3.69 (m, 1H), 3.40-3.48 (m, 6H), 2.16-2.63 (m, 8H).

### Synthetic Example 46: Synthesis of 4-(2-(3-(5-azaspiro[2.3]hexan-5-yl)azetidin-1-yl)-4-((1R,5S)-3,8-diazabicyclo[3.2.1]octan-3-yl)-8-fluoro-6-(trifluoromethyl)quinazolin-7-yl)-2-amino-7-fluorobenzo[b]thiophene-3-carbonitrile (Compound 363)

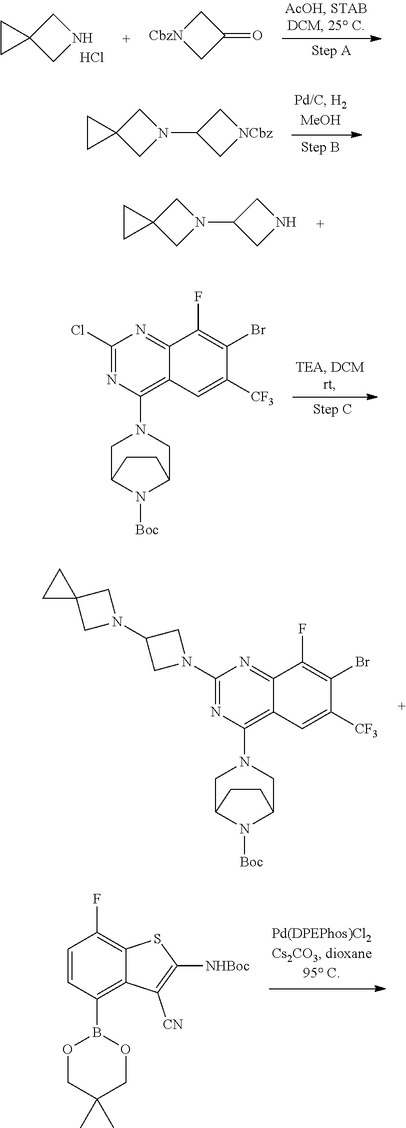

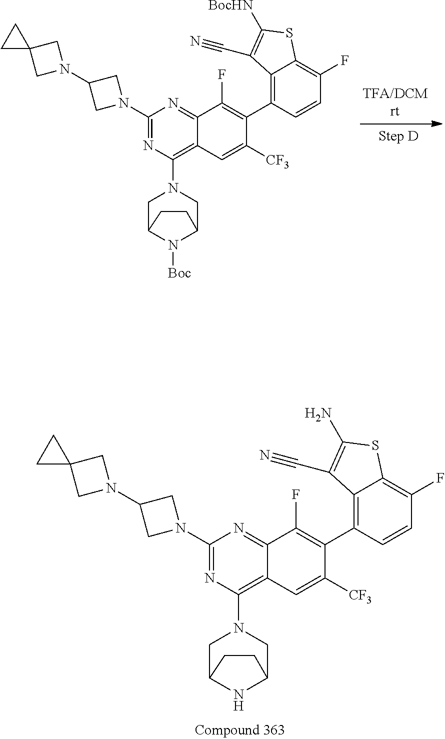 [SMILES: CC(C)(C)OC(=O)Nc1sc2c(F)ccc(-c3c(C(F)(F)F)cc4c(N5CC6CCC(C5)N6C(=O)OC(C)(C)C)nc(N5CC(N6CC7(CC7)C6)C5)nc4c3F)c2c1C#N ; N#Cc1c(N)sc2c(F)ccc(-c3c(C(F)(F)F)cc4c(N5CC6CCC(C5)N6)nc(N5CC(N6CC7(CC7)C6)C5)nc4c3F)c12]

Step A: Preparation of benzyl 3-(5-azaspiro[2.3]hexan-5-yl)azetidine-1-carboxylate: 5-azaspiro[2.3]hexane hydrochloride (1.00 eq, 0.6 g, 4.98 mmol) in DCM (30 mL) was added benzyl 3-oxoazetidine-1-carboxylate (3.00 eq, 3.06 g, 14.9 mmol) and acetic acid (3.00 eq, 0.85 mL, 14.9 mmol). After stirring for 10 minutes at room temperature, sodium triacetoxyborohydride (3.00 eq, 3.16 g, 14.9 mmol) was added and stirred at room temperature for 36 hours. The reaction was quenched with water and extracted with EtOAc. The organic layers were washed with saturated sodium bicarbonate solution, dried over sodium sulfate, filtered and concentrated under reduced pressure. The residue was purified by flash chromatography on silica gel, eluting with 50% EtOAc in petroleum ether to give benzyl 3-(5-azaspiro[2.3]hexan-5-yl)azetidine-1-carboxylate (920 mg, 67.1%).

Step B: Preparation of 5-(azetidin-3-yl)-5-azaspiro [2.3] hexane: To the solution of benzyl 3-(5-azaspiro[2.3]hexan-5-yl)azetidine-1-carboxylate (1.00 eq, 920 mg, 3.38 mmol) in methanol (40 mL) was added 10% Pd/C (184 mg). The mixture was stirred under hydrogen at 1 atmosphere for 12 hours. Catalyst was removed by filtration, washed with methanol. Solvent was removed under reduced pressure to give 5-(azetidin-3-yl)-5-azaspiro[2.3]hexane (450 mg, 96%). LCMS (ES+) m/z 139.2 (M+H).

Step C: Preparation of tert-butyl (1R,5S)-3-(2-(3-(5-azaspiro[2.3]hexan-5-yl)azetidin-1-yl)-7-(2-((tert-butoxycarbonyl)amino)-3-cyano-7-fluorobenzo[b]thiophen-4-yl)-8-fluoro-6-(trifluoromethyl)quinazolin-4-yl)-3,8-diazabicyclo[3.2.1]octane-8-carboxylate: A solution of tert-butyl 3-[2-[3-(5-azaspiro[2.3]hexan-5-yl)azetidin-1-yl]-7-bromo-8-fluoro-6-(trifluoromethyl)quinazolin-4-yl]-3,8-diazabicyclo[3.2.1]octane-8-carboxylate (150 mg, 0.234 mmol), tert-butyl (3-cyano-4-(5,5-dimethyl-1,3,2-dioxaborinan-2-yl)-7-fluorobenzo[b]thiophen-2-yl)carbamate (283 mg, 0.70 mmol), Cs₂CO₃ (228 mg, 0.70 mmol) and Pd(DPEPphos)Cl₂ (26 mg, 0.023 mmol) in dioxane (16 mL) was stirred at 90° C. for 8 hours. After cooled to ambient temperature, the reaction was concentrated to dryness and the residue was taken up in EtOAc (20 mL) and the organic layers washed with saturated brine solution. The organic layers were then separated, dried (MgSO₄), filtered and concentrated under reduced pressure. The crude was then purified by Prep-TLC (DCM/MeOH=15/1) and Preparative RP-HPLC to give tert-butyl (1R,5S)-3-(2-(3-(5-azaspiro[2.3]hexan-5-yl)azetidin-1-yl)-7-(2-((tert-butoxycarbonyl)amino)-3-cyano-7-fluorobenzo[b]thiophen-4-yl)-8-fluoro-6-(trifluoromethyl)quinazolin-4-yl)-3,8-diazabicyclo[3.2.1]octane-8-carboxylate (65 mg, 32%). LCMS (ES+) m/z 853.5 (M+H).

Step D: Preparation of 4-(2-(3-(5-azaspiro[2.3]hexan-5-yl)azetidin-1-yl)-4-((1R,5S)-3,8-diazabicyclo[3.2.1]octan-3-yl)-8-fluoro-6-(trifluoromethyl)quinazolin-7-yl)-2-amino-7-fluorobenzo[b]thiophene-3-carbonitrile: To a solution of tert-butyl (1R,5S)-3-(2-(3-(5-azaspiro[2.3]hexan-5-yl)azetidin-1-yl)-7-(2-((tert-butoxycarbonyl)amino)-3-cyano-7-fluorobenzo[b]thiophen-4-yl)-8-fluoro-6-(trifluoromethyl)quinazolin-4-yl)-3,8-diazabicyclo[3.2.1]octane-8-carboxylate (1.0 eq, 65 mg, 0.076 mmol) in DCM (2 mL) was added TFA (1 mL) at ambient temperature and stirred at ambient temperature for 2 hours. The solvent was removed by rotary evaporation and purified by Preparative RP-HPLC to give 4-(2-(3-(5-azaspiro[2.3]hexan-5-yl)azetidin-1-yl)-4-((1R,5S)-3,8-diazabicyclo[3.2.1]octan-3-yl)-8-fluoro-6-(trifluoromethyl)quinazolin-7-yl)-2-amino-7-fluorobenzo[b]thiophene-3-carbonitrile (27.5 mg, 56%). LCMS ESI (+) m/z 653.3 (M+H). ¹HNMR (400 MHz, CD₃OD) δ 88.00 (s, 1H), 7.19 (t, J=8.4 Hz, 1H), 7.01 (t, J=8.0 Hz, 1H), 4.54-4.68 (m, 5H), 4.21-4.35 (m, 8H), 3.84 (dd, J=26.0, 14.0 Hz, 2H), 2.12 (s, 4H), 0.86 (s, 4H).

### Synthetic Example 47: 4-(4-((1R,5S)-3,8-diazabicyclo[3.2.1]octan-3-yl)-8-chloro-6-(difluoromethyl)-2-(((2R,7aS)-2-fluorotetrahydro-1H-pyrrolizin-7a(5H)-yl)methoxy)quinazolin-7-yl)-7-fluorobenzo[d]thiazol-2-amine (Compound 368)

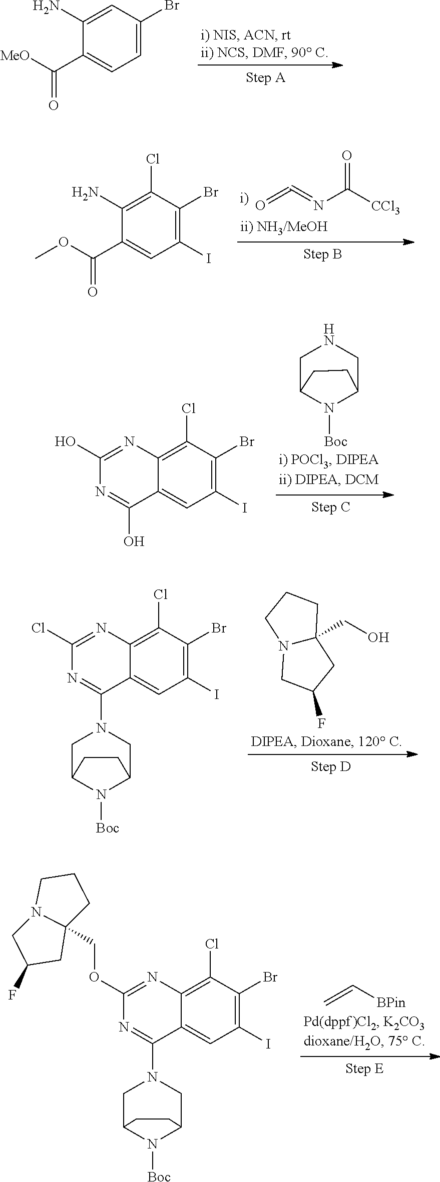

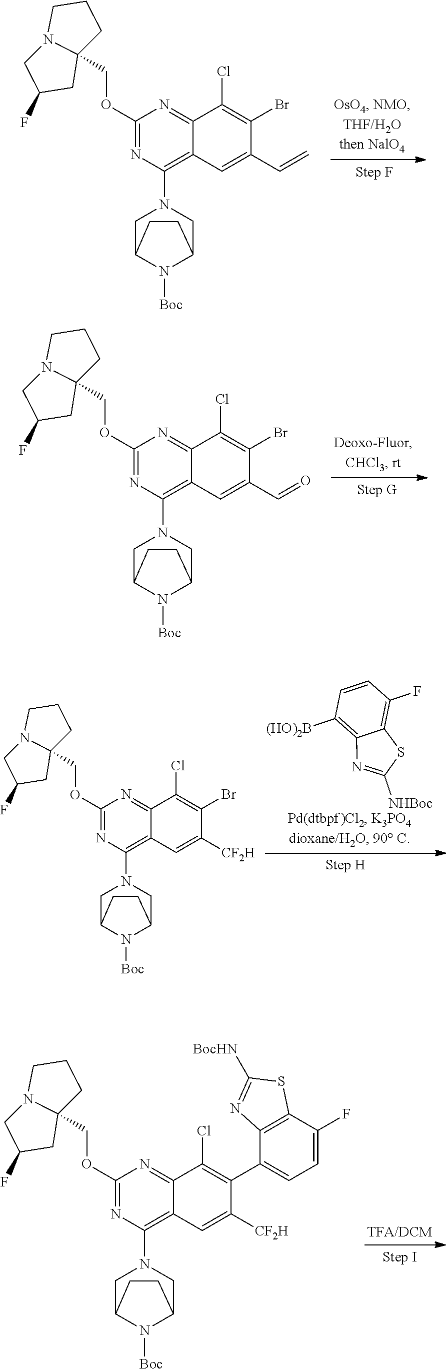 [SMILES: C=Cc1cc2c(N3CC4CCC(C3)N4C(=O)OC(C)(C)C)nc(OC[C@@]34CCCN3C[C@H](F)C4)nc2c(Cl)c1Br ; CC(C)(C)OC(=O)N1C2CCC1CN(c1nc(OC[C@@]34CCCN3C[C@H](F)C4)nc3c(Cl)c(Br)c(C=O)cc13)C2 ; CC(C)(C)OC(=O)N1C2CCC1CN(c1nc(OC[C@@]34CCCN3C[C@H](F)C4)nc3c(Cl)c(Br)c(C(F)F)cc13)C2 ; CC(C)(C)OC(=O)Nc1nc2c(B(O)O)ccc(F)c2s1 ; CC(C)(C)OC(=O)Nc1nc2c(-c3c(C(F)F)cc4c(N5CC6CCC(C5)N6C(=O)OC(C)(C)C)nc(OC[C@@]56CCCN5C[C@H](F)C6)nc4c3Cl)ccc(F)c2s1]

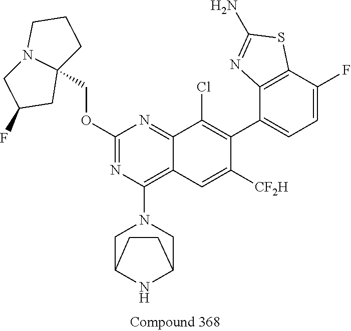 [SMILES: Nc1nc2c(-c3c(C(F)F)cc4c(N5CC6CCC(C5)N6)nc(OC[C@@]56CCCN5C[C@H](F)C6)nc4c3Cl)ccc(F)c2s1]

Step A: Preparation of methyl 2-amino-4-bromo-3-chloro-5-iodobenzoate: Methyl 2-amino-4-bromo-benzoate (5.0 g, 22 mmol) was taken in acetonitrile (50 mL), then a solution of N-iodosuccinimide (7.33 g, 33 mmol) was added at rt under argon. The reaction mixture was stirred at rt for 72 h. The resulting mixture was concentrated in rotavapor under vacuum. The residue obtained was dissolved in EtOAc (200 mL) and washed with water (3×50 mL) and sodium thiosulphate solution (100 mL). The organic layers were combined and dried over sodium sulphate and evaporated under vacuum to give methyl 2-amino-4-bromo-5-iodo-benzoate (7.2 g, 93%) as a brown solid. LCMS (ESI+): m/z 356.31 [M+H]. ¹H NMR (301 MHz, CDCl₃) δ 8.23 (s, 1H), 7.01 (s, 1H), 5.79 (s, 2H), 3.86 (s, 3H). To a solution of methyl 2-amino-4-bromo-5-iodo-benzoate (3.0 g, 8.43 mmol) in DMF (30 mL) was added N-chlorosuccinimide (1.01 g, 7.60 mmol) dropwise. The resulting mixture was stirred at 90° C. for 48 h then diluted with EtOAc (50 mL), washed with water (3×50 mL) and brine (30 mL). The organic layer was dried over sodium sulphate and evaporated to afford the crude product. The crude product was purified by solid phase silica column chromatography using 0-20% EtOAc in hexane as eluent to give methyl 2-amino-4-bromo-3-chloro-5-iodo-benzoate (1.64 g, 50%). LCMS (ESI+): m/z 410.94 [M]. ¹H NMR (300 MHz, CDCl₃) δ 8.28 (s, 1H), 6.49 (s, 2H), 3.89 (s, 3H).

Step B: Preparation of 7-bromo-8-chloro-6-iodoquinazoline-2,4-diol: To a stirring solution of methyl 2-amino-4-bromo-5-(difluoromethoxy)-3-fluoro-benzoate (1.62 g, 4.2 mmol) in THE (15 mL), 2,2,2-trichloroacetyl isocyanate (0.74 mL, 6.2 mmol) was added at room temperature under argon. The reaction was completed within 15 minutes of stirring at room temperature. The reaction mixture was concentrated under vacuum to give methyl 4-bromo-3-chloro-5-iodo-2-[(2,2,2-trichloroacetyl)carbamoylamino]benzoate (2.4 g, 100%) as a solid and used it for further step without any purification. MS (ESI+): 578.77 [M+H]. To a stirring suspension of methyl 4-bromo-3-chloro-5-iodo-2-[(2,2,2-trichloroacetyl)carbamoylamino]benzoate (2.5 g, 4.32 mmol) in methanol (11 mL) at room temperature, 7N NH₃ (1.4 mL, 10 mmol) in methanol was added and vigorously stirred the reaction mixture at room temperature for 17 h. The reaction mixture (white suspension) was concentrated under vacuo. The crude was triturated with diethyl ether (50 mL) and the solid was filtered and dried to give 7-bromo-8-chloro-6-iodo-quinazoline-2,4-diol (1.4 g, 81%) as a solid. ¹H NMR (300 MHz, DMSO-d6) δ 8.20 (s, 1H).

Step C: Preparation of tert-butyl (1R,5S)-3-(7-bromo-2,8-dichloro-6-iodoquinazolin-4-yl)-3,8-diazabicyclo[3.2.1]octane-8-carboxylate: Round bottom flask containing 7-bromo-8-chloro-6-iodo-quinazoline-2,4-diol (1.4 g, 3.5 mmol) was charged with phosphorous oxychloride (7.5 mL, 80 mmol), cooled to 0° C., and N,N-diisopropylethylamine (1.2 mL, 7.0 mmol) was added dropwise under argon atmosphere and maintained the stirring for 10 minutes. Then the reaction temperature was raised to room temperature to 110° C. After 1 hour, the reaction mixture was concentrated in vacuo and co-distilled with DCM multiple times to give 7-bromo-2,4,8-trichloro-6-iodo-quinazoline (1.5 g, 100%) as a light-yellow solid. This was directly taken to next step without any further purification. ¹H NMR (301 MHz, CDCl₃) δ 8.77 (s, 1H). To a solution of 7-bromo-2,4,8-trichloro-6-iodo-quinazoline (2.2 g, 5.02 mmol) in DCM (15 mL) at −45° C. was added N,N-diisopropylethylamine (2.6 mL, 15 mmol) followed by tert-butyl 3,8-diazabicyclo[3.2.1]octane-8-carboxylate (1.28 g, 6.02 mmol) and stirred at the same temperature for 2 h. The reaction was diluted with EtOAc (20 mL) and washed with NaHCO₃ (15 mL) solution. The EtOAc was collected, dried over sodium sulfate, filtered, and evaporated. The compound was purified on a 80 g column from 0 to 50% EtOAc in hexane as eluent to give tert-butyl (1R,5S)-3-(7-bromo-2,8-dichloro-6-iodoquinazolin-4-yl)-3,8-diazabicyclo[3.2.1]octane-8-carboxylate (1.45 g, 47%). LCMS (ESI+): 614.97 [M+H]. ¹H NMR (300 MHz, CDCl₃) δ 8.27 (s, 1H), 4.35 (s, 4H), 3.65 (d, J=43.2 Hz, 2H), 1.93 (d, J=6.7 Hz, 2H), 1.70 (d, J=14.2 Hz, 2H), 1.52 (s, 9H).

Step D: Preparation of tert-butyl (1R,5S)-3-(7-bromo-8-chloro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate: To a stirred solution of N,N-diisopropylethylamine (0.85 mL, 4.9 mmol) in 1,4-dioxane (20 mL) under an argon atmosphere were successively added [(2R,8S)-2-fluoro-1,2,3,5,6,7-hexahydropyrrolizin-8-yl]methanol (389 mg, 2.44 mmol) and tert-butyl 3-(7-bromo-2,8-dichloro-6-iodo-quinazolin-4-yl)-3,8-diazabicyclo[3.2.1]octane-8-carboxylate (1.0 g, 1.63 mmol). The reaction mixture was heated to 120° C. and stirred for 16 h. The mixture was concentrated to dryness in vacuo. The crude was then purified on 80 g silica gel column eluting from 0 to 100% EtOAc in hexane as eluent to give tert-butyl (1R,5S)-3-(7-bromo-8-chloro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate (0.36 g, 30%) as a foam solid. LCMS (ESI+): 736.16 [M+H]. ¹H NMR (300 MHz, CDCl₃) δ 8.19 (s, 1H), 5.19 (d, J=52.3 Hz, 1H), 4.43-4.24 (m, 4H), 4.16-4.10 (m, 2H), 3.64 (d, J=10.1 Hz, 2H), 3.23 (s, 2H), 3.17-3.11 (m, 1H), 2.98 (d, J=5.9 Hz, 1H), 2.31-2.15 (m, 3H), 2.0-1.89 (m, 5H), 1.72 (s, 2H), 1.51 (s, 9H).

Step E: Preparation of tert-butyl (1R,5S)-3-(7-bromo-8-chloro-2-(((2R,7aS)-2-fluorotetrahydro-1H-pyrrolizin-7a(5H)-yl)methoxy)-6-vinylquinazolin-4-yl)-3,8-diazabicyclo[3.2.1]octane-8-carboxylate: To a solution of tert-butyl (1R,5S)-3-(7-bromo-8-chloro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate (0.30 g, 0.41 mmol) in a mixture of water (0.2 mL) and 1,4-dioxane (2 mL) was added potassium carbonate (113 mg, 0.81 mmol). The resulting mixture was degassed with nitrogen for 5 minutes followed by the addition of 1,1′-bis(diphenylphosphino)ferrocenedichloropalladium(II) dichloromethane complex (30 mg, 0.041 mmol). The mixture was degassed with nitrogen for additional 20 minutes and 4,4,5,5-tetramethyl-2-vinyl-1,3,2-dioxaborolane (0.10 mL, 0.61 mmol) was added. The reaction mixture was heated to 75° C. and stirred for 4 h. The resulting mixture was cooled down to room temperature and extracted with EtOAc (50 mL). The organic layer was dried over sodium sulfate, filtered and evaporated under reduced pressure. The crude was purified on a 40 g column using (0-10%) MeOH in DCM as an eluent to give tert-butyl (1R,5S)-3-(7-bromo-8-chloro-2-(((2R,7aS)-2-fluorotetrahydro-1H-pyrrolizin-7a(5H)-yl)methoxy)-6-vinylquinazolin-4-yl)-3,8-diazabicyclo[3.2.1]octane-8-carboxylate. LCMS (ESI+): 636.17 [M+H]. ¹H NMR (300 MHz, CDCl₃) δ 7.82 (s, 1H), 7.22-7.10 (m, 1H), 5.63 (d, J=17.2 Hz, 1H), 5.40 (d, J=10.9 Hz, 1H), 5.21 (d, J=52.3 Hz, 1H), 4.44-4.26 (m, 5H), 4.17 (d, J=10.5 Hz, 1H), 3.70-3.51 (m, 2H), 3.33-3.24 (m, 2H), 3.17 (s, 1H), 3.00-2.95 (m, 1H), 2.35-2.19 (m, 3H), 2.00-1.86 (m, 5H), 1.75 (s, 2H), 1.53 (s, 9H).

Step F: Preparation of tert-butyl (1R,5S)-3-(7-bromo-8-chloro-2-(((2R,7aS)-2-fluorotetrahydro-1H-pyrrolizin-7a(5H)-yl)methoxy)-6-formylquinazolin-4-yl)-3,8-diazabicyclo[3.2.1]octane-8-carboxylate: To a mixture of tert-butyl 3-[7-bromo-8-chloro-2-[[(2R,8S)-2-fluoro-1,2,3,5,6,7-hexahydropyrrolizin-8-yl]methoxy]-6-vinyl-quinazolin-4-yl]-3,8-diazabicyclo[3.2.1]octane-8-carboxylate (0.24 g, 0.37 mmol) in THE (4 mL) and water (1 mL) were added osmium tetroxide (0.0035 mL, 0.011 mmol) followed by 4-methylmorpholine N-oxide (86 mg, 0.74 mmol) at room temperature. After 2 h, sodium periodate (118 mg, 0.55 mmol) was added to the reaction and stirred for 2 h. After completion of the reaction, the crude material was washed with water and brine and was extracted with EtOAc (60 mL). The organic layer was dried over Na₂SO₄ and concentrated to give crude residue which was purified by silica gel column chromatography on 12 g column using 0-10% MeOH in DCM as eluent to give tert-butyl (1R,5S)-3-(7-bromo-8-chloro-2-(((2R,7aS)-2-fluorotetrahydro-1H-pyrrolizin-7a(5H)-yl)methoxy)-6-formylquinazolin-4-yl)-3,8-diazabicyclo[3.2.1]octane-8-carboxylate (125 mg, 53%). LCMS (ESI+): 638.09 [M+H]. ¹HNMR (300 MHz, CDCl₃) δ 10.38 (s, 1H), 8.31 (s, 1H), 5.28 (d, J=54.26 Hz, 1H), 4.50-4.33 (m, 2H), 4.30 (d, J=10.4 Hz, 2H), 4.17 (d, J=10.4 Hz, 1H), 3.66 (d, J=12.5 Hz, 2H), 3.24 (s, 2H), 3.14 (d, J=8.7 Hz, 1H), 3.00-2.92 (m, 1H), 2.33-2.13 (m, 3H), 1.92 (s, 4H), 1.69 (d, J=8.5 Hz, 2H), 1.60 (s, 2H), 1.52 (s, 9H).

Step G: Preparation of tert-butyl (1R,5S)-3-(7-bromo-8-chloro-6-(difluoromethyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate: To a solution of tert-butyl (1R,5S)-3-(7-bromo-8-chloro-2-(((2R,7aS)-2-fluorotetrahydro-1H-pyrrolizin-7a(5H)-yl)methoxy)-6-formylquinazolin-4-yl)-3,8-diazabicyclo[3.2.1]octane-8-carboxylate (120 mg, 0.20 mmol) in chloroform (4 mL) was added deoxo-fluor (0.35 mL, 1.90 mmol) at room temperature. The mixture was stirred at room temperature for 16 h, then quenched with water (2 mL), and extracted with EtOAc (10 mL). The organic layers were combined, dried over sodium sulfate, filtered, and evaporated under reduced pressure. The crude material was purified on a 12 g column using 0 to 10% MeOH in DCM as eluent to give tert-butyl (1R,5S)-3-(7-bromo-8-chloro-6-(difluoromethyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (102 mg, 82%). LCMS ESI (+): 660.3 [M+H]. ¹H NMR (301 MHz, CDCl₃) δ 7.98 (s, 1H), 7.00 (t, J=55.1 Hz, 1H), 5.22 (s, 1H), 4.40-4.32 (m, 4H), 4.21 (d, J=10.6 Hz, 1H), 3.58 (d, J=13.6 Hz, 3H), 3.40-3.29 (m, 3H), 3.21 (s, 1H), 3.01 (s, 1H), 2.35-2.17 (m, 3H), 1.95 (d, J=8.7 Hz, 4H), 1.75 (d, J=8.0 Hz, 2H), 1.54 (d, J=1.7 Hz, 9H).

Step H: Preparation of tert-butyl (1R,5S)-3-(7-(2-((tert-butoxycarbonyl)amino)-7-fluorobenzo[d]thiazol-4-yl)-8-chloro-6-(difluoromethyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate: To a mixture of tert-butyl (1R,5S)-3-(7-bromo-8-chloro-6-(difluoromethyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (35 mg, 0.053 mmol) and [2-(tert-butoxycarbonylamino)-7-fluoro-1,3-benzothiazol-4-yl]boronic acid (33 mg, 0.11 mmol) in a mixture of 1,4-dioxane (1 mL) and water (0.2 mL) was added potassium phosphate tribasic (14 mg, 0.11 mmol) under argon. The resulting mixture was degassed for 10 mins, then 1,1′-bis(di-t-butylphosphino)ferrocene palladium dichloride (5.2 mg, 0.008 mmol) was added and the mixture was degassed again and heated at 90° C. for 3 hrs. The resulting mixture was cooled down to room temperature and extracted with EtOAc (10 mL) and water (5 mL). The organic layer was collected, dried over sodium sulfate, filtered and evaporated under reduced pressure. The resulting crude material was purified on a 12 g column using 0-10% MeOH/DCM as eluent to provide tert-butyl (1R,5S)-3-(7-(2-((tert-butoxycarbonyl)amino)-7-fluorobenzo[d]thiazol-4-yl)-8-chloro-6-(difluoromethyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (8.0 mg, 18%). LCMS (ESI+): m/z 848.43 (M+H). ¹HNMR (300 MHz, CDCl₃) δ 8.08 (s, 1H), 7.22 (d, J=6.0 Hz, 1H), 7.11 (t, J=8.6 Hz, 1H), 6.22 (t, J=55.5 Hz, 1H), 5.24 (d, J=52 Hz, 1H), 4.44-4.29 (m, 5H), 4.20 (s, 1H), 3.63 (s, 2H), 3.24 (d, J=12.1 Hz, 2H), 3.14 (s, 1H), 2.95 (s, 1H), 2.22 (d, J=25.1 Hz, 3H), 1.90 (s, 4H), 1.80 (d, J=8.7 Hz, 2H), 1.65 (s, 2H), 1.53 (d, J=1.7 Hz, 18H).

Step I: Preparation of 4-(4-((1R,5S)-3,8-diazabicyclo[3.2.1]octan-3-yl)-8-chloro-6-(difluoromethyl)-2-(((2R,7aS)-2-fluorotetrahydro-1H-pyrrolizin-7a(5H)-yl)methoxy)quinazolin-7-yl)-7-fluorobenzo[d]thiazol-2-amine: To a stirred solution of tert-butyl (1R,5S)-3-(7-(2-((tert-butoxycarbonyl)amino)-7-fluorobenzo[d]thiazol-4-yl)-8-chloro-6-(difluoromethyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (8.0 mg, 0.0094 mmol) in DCM (1 mL) was added trifluoroacetic acid (0.30 mL, 4.0 mmol) at room temperature and stirred for 3 h at room temperature. The mixture was concentrated under reduced pressure, several times co-evaporated with DCM to give 4-(4-((1R,5S)-3,8-diazabicyclo[3.2.1]octan-3-yl)-8-chloro-6-(difluoromethyl)-2-(((2R,7aS)-2-fluorotetrahydro-1H-pyrrolizin-7a(5H)-yl)methoxy)quinazolin-7-yl)-7-fluorobenzo[d]thiazol-2-amine (9.6 mg, 92%). LCMS (ESI+): m/z 648.40 (M+H). ¹H NMR (300 MHz, CD₃OD) δ 8.23 (s, 1H), 7.21 (dd, J=8.3, 5.5 Hz, 1H), 7.14-7.06 (m, 1H), 6.59 (d, J=55.0 Hz, 1H), 5.58 (d, J=51.9 Hz, 1H), 4.82-4.70 (m, 4H), 4.27 (s, 2H), 4.14-3.91 (m, 5H), 3.49 (s, 1H), 2.80-2.59 (m, 2H), 2.48 (s, 1H), 2.37 (t, J=8.9 Hz, 2H), 2.14 (s, 5H).

### Synthetic Example 48: Synthesis of 4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)-6-methoxyquinazolin-7-yl)-7-fluorobenzo[d]thiazol-2-amine (Compound 369)

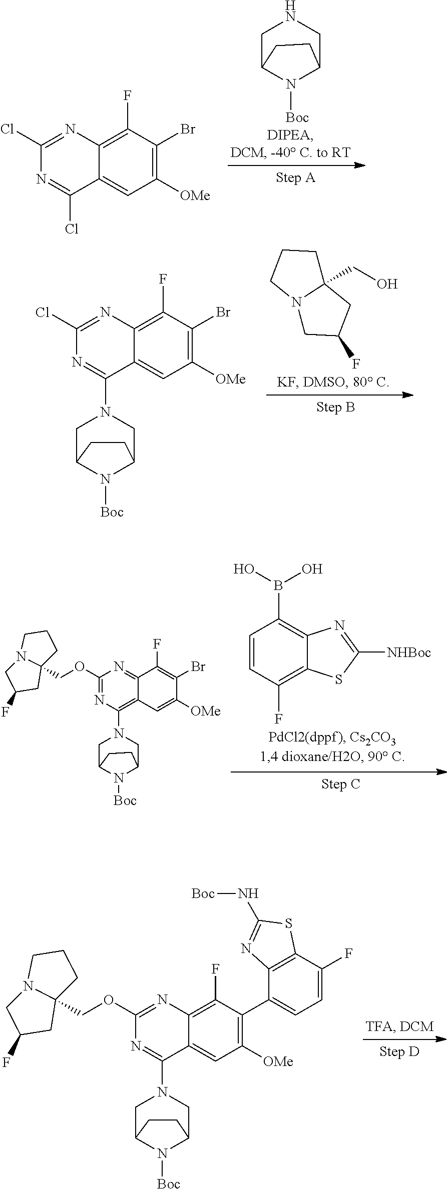

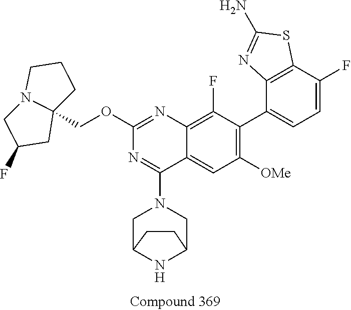 [SMILES: COc1cc2c(N3CC4CCC(C3)N4)nc(OC[C@@]34CCCN3C[C@H](F)C4)nc2c(F)c1-c1ccc(F)c2sc(N)nc12]

Step A: Preparation of tert-butyl (1R,5S)-8-(7-bromo-2-chloro-8-fluoro-6-methoxyquinazolin-4-yl)-3,8-diazabicyclo[3.2.1]octane-3-carboxylate. A solution of 7-bromo-2,4-dichloro-8-fluoro-6-methoxy-quinazoline (330 mg, 1.01 mmol) in DCM (6.6 mL) was cooled to −45° C. under nitrogen. To this, DIPEA (0.53 mL, 3.04 mmol) and tert-butyl 3,8-diazabicyclo[3.2.1]octane-8-carboxylate (258 mg, 1.21 mmol) were added subsequently. The reaction was warmed slowly to 0° C. After 30 minutes of stirring, the reaction mixture was diluted with DCM (60 mL). The organic layer was washed with water, dried over Na₂SO₄, filtered and concentrated. The crude was purified by flash chromatography on silica gel with a gradient of 0-70% EtOAc in hexanes to provide the title compound (350 mg, 69% yield). LCMS (ESI+) m/z 501.09 [M+H].

Step B: Preparation of tert-butyl (1R,5S)-8-(7-bromo-8-fluoro-2-(((2R,7aS)-2-fluorotetrahydro-1H-pyrrolizin-7a(5H)-yl)methoxy)-6-methoxyquinazolin-4-yl)-3,8-diazabicyclo[3.2.1]octane-3-carboxylate: To a stirring suspension of tert-butyl (1R,5S)-8-(7-bromo-2-chloro-8-fluoro-6-methoxyquinazolin-4-yl)-3,8-diazabicyclo[3.2.1]octane-3-carboxylate (70 mg, 0.140 mmol) in DMSO (1.3 mL), ((2R,7aS)-2-fluorotetrahydro-1H-pyrrolizin-7a(5H)-yl)methanol (67 mg, 0.419 mmol) and KF (65 mg, 1.12 mmol) were added subsequently at room temperature. The reaction mixture was heated to 80° C. After 24 hours of heating, the reaction was cooled to ambient temperature and water/EtOAc was added. Layers were separated and aqueous layer was further extracted with EtOAc (2×20 mL). The organic layers were combined and concentrated in vacuo. The resultant crude was purified by flash column chromatography on silica gel (fractions eluted in 4-10% DCM/methanol) to provide title compound (16 mg, 18% yield). MS (ESI+) m/z 624.38. ¹H NMR (300 MHz, CD₃OD) δ 7.09 (s, 1H), 5.34 (d, J=53.6 Hz, 1H), 4.46-4.33 (m, 4H), 4.30 (d, J=10.7 Hz, 1H), 4.23 (d, J=10.7 Hz, 1H), 3.99 (s, 3H), 3.58 (d, J=13.0 Hz, 2H), 3.41-3.28 (m, 1H), 3.15-2.99 (m, 1H), 2.48-2.11 (m, 4H), 2.10-1.74 (m, 8H), 1.52 (s, 9H).

Step C: Preparation of tert-butyl (1R,5S)-8-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-methoxyquinazolin-4-yl)-3,8-diazabicyclo[3.2.1]octane-3-carboxylate: To a stirring solution of tert-butyl (1R,5S)-8-(7-bromo-8-fluoro-2-(((2R,7aS)-2-fluorotetrahydro-1H-pyrrolizin-7a(5H)-yl)methoxy)-6-methoxyquinazolin-4-yl)-3,8-diazabicyclo[3.2.1]octane-3-carboxylate (16 mg, 0.0256 mmol) in 1,4-dioxane (1.5 mL), [2-(tert-butoxycarbonylamino)-7-fluoro-1,3-benzothiazol-4-yl]boronic acid (16 mg, 0.0512 mmol) and an aqueous solution of Cs₂CO₃ (17 mg, 0.0512 mmol) in water (0.15 mL) were added subsequently. The reaction mixture was degassed with argon for 10 minutes, then Pd(dppf)Cl₂·DCM was added and degassed again for 10 min. The reaction mixture was heated to 90° C. After 90 minutes of heating, the reaction mixture was cooled to room temperature and diluted with water/EtOAc. Layers were separated and the aqueous layer was further extracted with EtOAc (2×15 mL). The organic layers were combined, washed with brine, dried over Na₂SO₄, filtered and concentrated in vacuo. The resultant crude was purified by flash chromatography on silica gel (eluted with 60-72% EtOAc in hexane) to afford title compound (10.6 mg, 51%). MS (ESI+) m/z 812.56 [M+H]. ¹H NMR (300 MHz, CD₃OD) δ 7.35 (ddd, J=7.9, 5.4, 2.1 Hz, 1H), 7.18-7.06 (m, 2H), 5.28 (d, J=54.0 Hz, 1H), 4.51-4.32 (m, 4H), 4.23 (qd, J=10.5, 2.0 Hz, 2H), 3.77 (d, J=7.0 Hz, 3H), 3.68-3.44 (m, 2H), 3.28-3.12 (m, 2H), 3.08-2.94 (m, 1H), 2.39-2.06 (m, 4H), 2.06-1.77 (m, 7H), 1.57-1.48 (m, 18H).

Step D: Preparation of 4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)-6-methoxyquinazolin-7-yl)-7-fluorobenzo[d]thiazol-2-amine: To a stirring solution of tert-butyl (1R,5S)-8-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-methoxyquinazolin-4-yl)-3,8-diazabicyclo[3.2.1]octane-3-carboxylate (10 mg, 0.0128 mmol) in DCM (3 mL), TFA was added at room temperature. After 3 hours of stirring at room temperature, the reaction mixture was concentrated in vacuo and co-distilled with DCM three times and dried under high vacuum. The product obtained was triturated with diethyl ether, decanted and dried to afford title compound as TFA salt (13.1 mg, quantitative). MS (ESI+) m/z 612.45 [M+H]. ¹H NMR (300 MHz, CD₃OD) δ 7.30 (dd, J=8.4, 5.2 Hz, 1H), 7.17 (s, 1H), 7.09 (t, J=8.8 Hz, 1H), 5.69-5.42 (m, 1H), 4.80-4.61 (m, 4H), 4.26 (s, 2H), 4.11-3.74 (m, 7H), 3.54-3.40 (m, 1H), 2.81-2.71 (m, 1H), 2.61-2.25 (m, 5H), 2.16 (s, 5H).

### Synthetic Example 49: Synthesis of 4-(4-((1R,5S)-3,8-diazabicyclo[3.2.1]octan-3-yl)-6-(difluoromethyl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine (Compound 370)

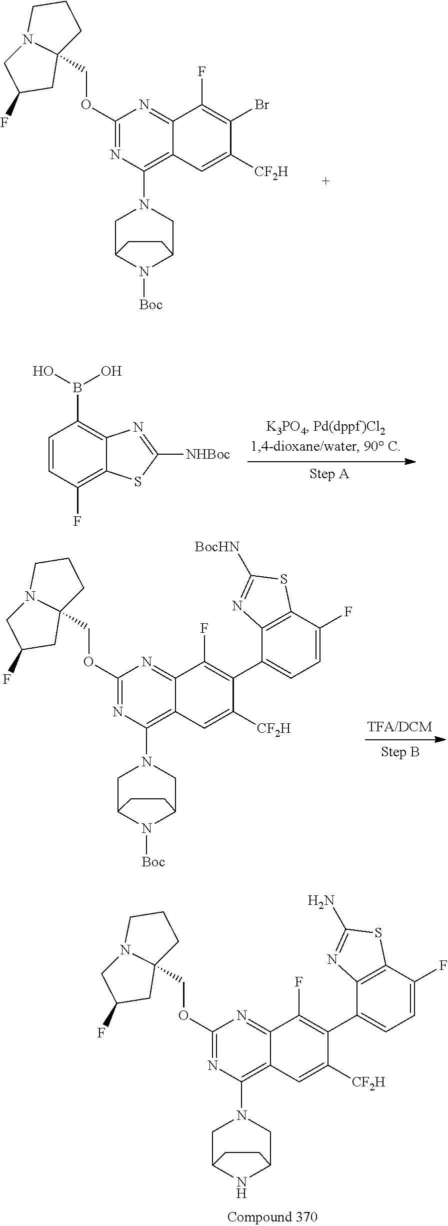

Step A: Preparation of tert-butyl 3-[7-[2-(tert-butoxycarbonylamino)-7-fluoro-1,3-benzothiazol-4-yl]-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate: To a mixture of tert-butyl 3-[7-bromo-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (17 mg, 0.026 mmol), [2-(tert-butoxycarbonylamino)-7-fluoro-1,3-benzothiazol-4-yl]boronic acid (2.00 eq, 16 mg, 0.0528 mmol) and potassium phosphate tribasic (7.2 mg, 0.053 mmol) in 1,4-dioxane (1.0 mL) and water (0.20 mL) under nitrogen was added Pd(dtbpf)Cl₂ (2.6 mg, 0.004 mmol) and heated at 90° C. for 3 h. The reaction mixture was extracted with EA/water. The EA was collected, dried (Na₂SO₄), filtered and evaporated. The mixture was purified by silica gel column chromatography (25 g) using MeOH/DCM (0-10%) as eluent to give mixture which was repurified by reverse phase chromatography using ACN/0.1% HCOOH buffer (5-95%) to give tert-butyl 3-[7-[2-(tert-butoxycarbonylamino)-7-fluoro-1,3-benzothiazol-4-yl]-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (7.0 mg, 32%). LCMS ESI (+) m/z 832.50 (M+H). ¹H NMR (300 MHz, CD₃OD) δ 8.10 (s, 1H), 7.45-7.38 (m, 1H), 7.21 (t, J=8.8 Hz, 1H), 6.57 (t, J=55.2 Hz, 2H), 5.50 (d, J=52.6 Hz, 1H), 4.66-4.47 (m, 4H), 4.48-4.35 (m, 2H), 3.74 (d, J=11.1 Hz, 5H), 2.69-2.44 (m, 2H), 2.42-2.10 (m, 4H), 2.09-1.97 (m, 2H), 1.86-1.73 (m, 2H), 1.55 (s, 18H).

Step B: Preparation of 4-[4-(3,8-diazabicyclo[3.2.1]octan-3-yl)-6-(difluoromethyl)-8-fluoro-2-[[(2R,8S)-2-fluoro-1,2,3,5,6,7-hexahydropyrrolizin-8-yl]methoxy]quinazolin-7-yl]-7-fluoro-1,3-benzothiazol-2-amine: To a stirred solution of tert-butyl 3-[7-[2-(tert-butoxycarbonylamino)-7-fluoro-1,3-benzothiazol-4-yl]-6-(difluoromethyl)-8-fluoro-2-[[(2R,8S)-2-fluoro-1,2,3,5,6,7-hexahydropyrrolizin-8-yl]methoxy]quinazolin-4-yl]-3,8-diazabicyclo[3.2.1]octane-8-carboxylate (7.5 mg, 0.009 mmol) in DCM (1.0 mL) was added trifluoroacetic acid (0.30 mL, 3.89 mmol) at rt and stirred for 3 h at rt. After completion of the reaction it was evaporated under reduced pressure, several times co evaporated with DCM to give 4-[4-(3,8-diazabicyclo[3.2.1]octan-3-yl)-6-(difluoromethyl)-8-fluoro-2-[[(2R,8S)-2-fluoro-1,2,3,5,6,7-hexahydropyrrolizin-8-yl]methoxy]quinazolin-7-yl]-7-fluoro-1,3-benzothiazol-2-amine; 2,2,2-trifluoroacetic acid (8.8 mg, 90%). LCMS ESI (+) m/z 632.43 (M+H). ¹H NMR (300 MHz, CD₃OD) δ 8.10 (s, 1H), 7.27 (dd, J=8.5, 5.3 Hz, 1H), 7.06 (dd, J=9.2, 8.5 Hz, 1H), 6.61 (t, J=55.0 Hz, 1H), 5.57 (d, J=51.6 Hz, 1H), 4.80-4.70 (m, 3H), 4.26 (s, 2H), 4.03-3.94 (m, 4H), 3.93-3.82 (m, 2H), 3.50-3.42 (m, 1H), 2.83-2.69 (m, 1H), 2.63-2.55 (m, 1H), 2.48-2.29 (m, 3H), 2.23-2.06 (m, 5H).

A compound of present disclosure, such as a compound of a formula included in any of Tables 2-8, may be synthesized according to one of the general routes outlined in Synthetic Examples 1-31 or by various other methods generally known in the art.

Table 2 includes selected compounds of the present disclosure.

TABLE 2
Selected compounds of the present disclosure.

| Compound | | |
|---|---|---|
| No. | Structure | Analytical data |
| 1 | 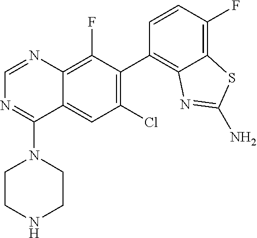 [SMILES: Nc1nc2c(-c3c(Cl)cc4c(N5CCNCC5)ncnc4c3F)ccc(F)c2s1] | LCMS m/z [M + 1]: 433.1 ¹HNMR (400 MHz, CD₃OD): δ 8.79 (s, 1H), 8.07 (s, 1H), 7.26 (t, J = 8.0 Hz, 1H), 7.03 (t, J = 8.8 Hz, 1H), 4.18 (t, J = 4.8 Hz, 4H), 3.49 (t, J = 5.2 Hz, 4H). |
| 2 | 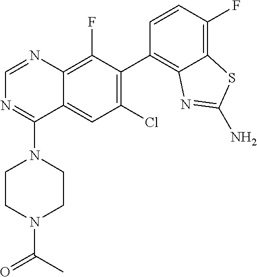 [SMILES: CC(=O)N1CCN(c2ncnc3c(F)c(-c4ccc(F)c5sc(N)nc45)c(Cl)cc23)CC1] | LCMS m/z [M + 1]: 475.3 ¹HNMR (400 MHz, CD₃OD): δ 8.73 (s, 1H), 8.22 (s, 1H), 7.27-7.30 (m, 1H), 7.04 (t, J = 8.8 Hz, 1H), 4.34 (d, J = 5.6 Hz, 4H), 3.88 (d, J = 5.6 Hz, 4H), 2.18 (s, 3H). |
| 3 | 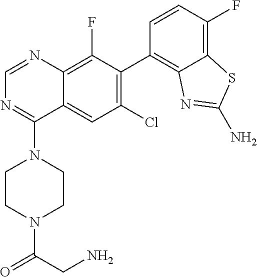 [SMILES: NCC(=O)N1CCN(c2ncnc3c(F)c(-c4ccc(F)c5sc(N)nc45)c(Cl)cc23)CC1] | LCMS m/z [M + 1]: 490.2 ¹HNMR (400 MHz, CD₃OD): δ 8.67 (s, 1H), 8.02 (s, 1H), 7.23-7.26 (m, 1H), 7.01(t, J = 8.8 Hz, 1H), 4.01 (m, 4H), 3.87-3.90 (m, 4H), 3.72 (s, 2H). |
| 4 | 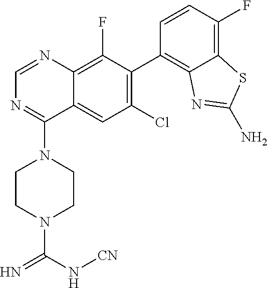 [SMILES: N#CNC(=N)N1CCN(c2ncnc3c(F)c(-c4ccc(F)c5sc(N)nc45)c(Cl)cc23)CC1] | LCMS m/z [M + 1]: found 500.1 ¹HNMR (400 MHz, CD₃OD): δ 8.65 (s, 1H), 8.13 (s, 1H), 7.20-7.30 (m, 1H), 7.00 (t, J = 8.8 Hz, 1H), 3.98-4.15 (m, 4H), 3.75-3.85 (m, 4H). |
| 5 | 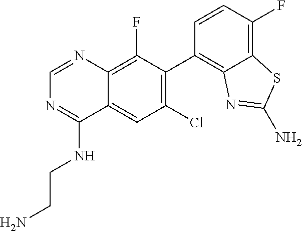 [SMILES: NCCNc1ncnc2c(F)c(-c3ccc(F)c4sc(N)nc34)c(Cl)cc12] | LCMS m/z [M + 1]: 407.1 ¹HNMR (400 MHz, CD₃OD): δ 8.56 (s, 1H), 8.17 (s, 1H), 7.23 (dd, J = 8.4, 5.5 Hz, 1H), 7.01 (t, J = 8.8 Hz, 1H), 3.95 (t, J = 5.8 Hz, 2H), 3.28 (t, J = 5.8 Hz, 2H). |
| 6 | 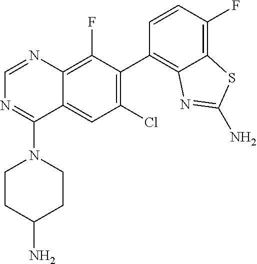 [SMILES: Nc1nc2c(-c3c(Cl)cc4c(N5CCC(N)CC5)ncnc4c3F)ccc(F)c2s1] | LCMS m/z [M + 1]: 447.2 ¹H NMR (400 MHz, CD₃OD): δ 8.66 (s, 1H), 7.95 (s, 1H), 7.24 (dd, J = 8.3, 5.5 Hz, 1H), 7.00 (t, J = 8.8 Hz, 1H), 4.57 (d, J = 12.4 Hz, 2H), 3.37-3.52 (m, 3H), 2.19 (d, J = 11.2 Hz, 2H), 1.82-1.88 (m, 2H). |
| 7 | 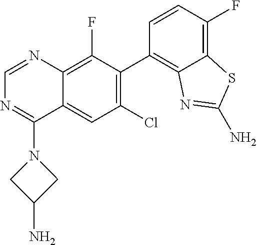 [SMILES: Nc1nc2c(-c3c(Cl)cc4c(N5CC(N)C5)ncnc4c3F)ccc(F)c2s1] | LCMS m/z [M + 1]: 419.2 ¹H NMR (400 MHz, CD₃OD): δ 8.50 (s, 1H), 7.88 (s, 1H), 7.23 (dd, J = 8.3, 5.5 Hz, 1H), 7.00 (t, J = 8.8 Hz, 1H), 4.89 (d, J = 18.6 Hz, 2H), 4.40-4.52 (m, 2H), 4.21-4.31 (m, 1H). |
| 8 | 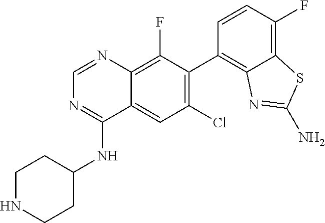 [SMILES: Nc1nc2c(-c3c(Cl)cc4c(NC5CCNCC5)ncnc4c3F)ccc(F)c2s1] | LCMS m/z [M + 1]: 447.2 ¹HNMR (400 MHz, CD₃OD): δ 8.52(s, 1H), 8.31(s, 1H), 7.21-7.24 (m, 1H), 6.99(t, J = 8.8 Hz, 1H ), 4.53-4.58 (m, 1H), 3.49-3.52 (m, 2H), 3.14-3.20 (m, 2H), 2.31-2.35 (m, 2H), 1.91-2.00 (m, 2H) |
| 9 | 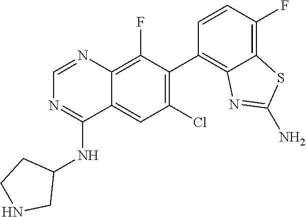 [SMILES: Nc1nc2c(-c3c(Cl)cc4c(NC5CCNC5)ncnc4c3F)ccc(F)c2s1] | LCMS m/z [M + 1]: 433.1 ¹HNMR (400 MHz, CD₃OD): δ 8.79 (s, 1H), 8.51 (s, 1H), 7.21-7.45 (m, 1H), 7.06 (t, J = 8.8 Hz, 1H), 5.07-5.22 (m, 1H), 3.76- 3.88 (m, 1H), 3.45-3.75 (m, 3H), 2.52-2.66 (m, 1H), 2.35-2.48 (m, 1H). |
| 10 | 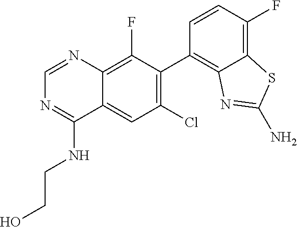 [SMILES: Nc1nc2c(-c3c(Cl)cc4c(NCCO)ncnc4c3F)ccc(F)c2s1] | LCMS m/z [M + 1]: 408.1 ¹HNMR (400 MHz, DMSO-d6): δ 8.51-8.54 (m, 2H), 8.41(s, 1H), 7.90 (s, 2H), 7.24- 7.27(dd, J = 8.4, 5.6 Hz, 1H), 7.04-7.08 (m, 1H), 4.80-4.90 (m, 1H), 3.61-3.70 (s, 4H). |
| 11 | 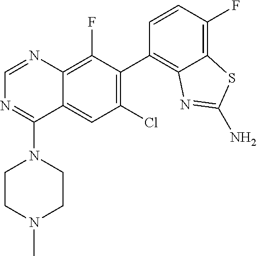 [SMILES: CN1CCN(c2ncnc3c(F)c(-c4ccc(F)c5sc(N)nc45)c(Cl)cc23)CC1] | LCMS m/z [M + 1]: 447.2 ¹HNMR (400 MHz, CD₃OD): δ 8.73 (s, 1H), 8.01 (s, 1H), 7.24 (dd, J = 8.4, 5.2 Hz, 1H), 7.01 (t, J = 8.8 Hz, 1H), 4.08-4.15 (m, 4H), 3.22 (t, J = 4.8 Hz, 4H), 2.78 (s, 3H). |
| 12 | 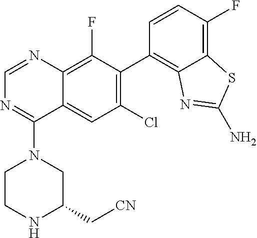 [SMILES: N#CC[C@H]1CN(c2ncnc3c(F)c(-c4ccc(F)c5sc(N)nc45)c(Cl)cc23)CCN1] | LCMS m/z [M + 1]: 472.2 ¹H NMR (400 MHz, CD₃OD) δ 8.66 (s, 1H), 7.97 (s, 1H), 7.23-7.26 (m, 1H), 7.00 (t, J = 8.8 Hz, 1H), 4.48 (t, J = 12.0 Hz, 1H), 4.32 (d, J = 12.0 Hz, 1H), 3.44-3.54 (m, 1H), 3.13-3.26 (m, 3H), 2.99-3.08 (m, 1H), 2.72- 2.74 (m, 2H). |
| 12a | 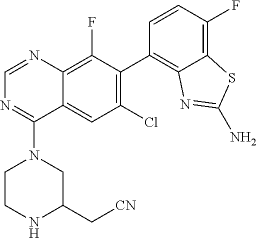 [SMILES: N#CCC1CN(c2ncnc3c(F)c(-c4ccc(F)c5sc(N)nc45)c(Cl)cc23)CCN1] | |
| 13 | 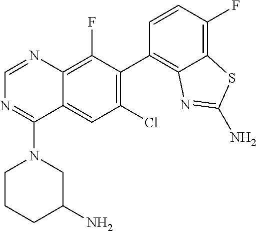 [SMILES: Nc1nc2c(-c3c(Cl)cc4c(N5CCCC(N)C5)ncnc4c3F)ccc(F)c2s1] | LCMS m/z [M + 1]: 447.2 ¹HNMR (400 MHz, CD₃OD): δ 8.68 (s, 1H), 7.99 (s, 1H), 7.23-7.27 (m, 1H), 7.00 (t, J = 8.0 Hz, 1H), 4.35 (t, J = 10.4 Hz, 1H), 4.15(d, J = 13.2 Hz, 1H), 3.66-3.58 (m, 2H), 3.55-3.52 (m, 1H), 2.21(d, J = 10.8 Hz, 1H), 1.96-1.99 (m, 1H), 1.75-1.86 (m, 1H). |
| 14 | 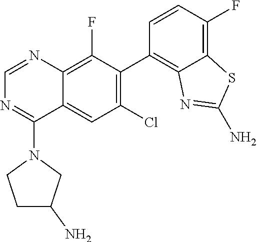 [SMILES: Nc1nc2c(-c3c(Cl)cc4c(N5CCC(N)C5)ncnc4c3F)ccc(F)c2s1] | LCMS m/z [M + 1]: 433.2 ¹HNMR (400 MHz, CD₃OD): δ 8.51 (s, 1H), 8.24 (s, 1H), 7.21-7.25 (m, 1H), 7.00 (t, J = 8.0 Hz, 1H), 4.25-4.29 (m, 2H), 4.17 (d, J = 6.0 Hz, 1H), 3.99 (d, J = 8.80 Hz, 2H), 2.48 (dd, J = 13.2, 6.0 Hz, 1H), 2.21 (t, J = 5.60 Hz, 1H) |
| 15 | 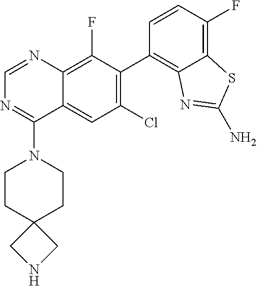 [SMILES: Nc1nc2c(-c3c(Cl)cc4c(N5CCC6(CC5)CNC6)ncnc4c3F)ccc(F)c2s1] | LCMS m/z [M + 1]: 473.1 ¹HNMR (400 MHz, CD₃OD): δ 8.62 (s, 1H), 8.55 (s, 1H), 7.93 (s, 1H), 7.24 (dd, J = 8.4, 5.2 Hz, 1H), 7.00 (t, J = 8.8 Hz, 1H), 4.50- 4.65 (m, 2H), 3.87-3.95 (m, 7H), 1.92-2.20 (m, 4H). |
| 16 | 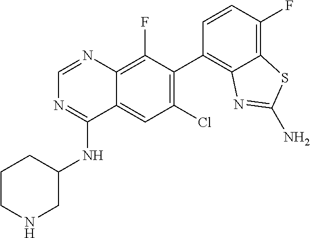 [SMILES: Nc1nc2c(-c3c(Cl)cc4c(NC5CCCNC5)ncnc4c3F)ccc(F)c2s1] | LCMS m/z [M + 1]: 447.1 ¹HNMR (400 MHz, CD₃OD): δ 8.69 (s, 1H), 8.40 (s, 1H), 7.20-7.30 (m, 1H), 7.02 (t, J = 8.8 Hz, 1H), 3.70-3.76 (m, 1H), 3.39- 3.49 (m, 1H), 2.97-3.07 (m, 2H), 2.21-2.30 (m, 1H), 2.10-2.20 (m, 1H), 1.85-1.96 (m, 2H). |
| 17 | 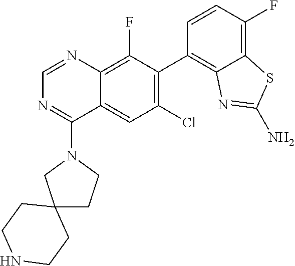 [SMILES: Nc1nc2c(-c3c(Cl)cc4c(N5CCC6(CCNCC6)C5)ncnc4c3F)ccc(F)c2s1] | LCMS m/z [M + 1]: 487.3 ¹HNMR (400 MHz, CD₃OD) δ: 8.49 (s, 1H), 8.26 (s, 1H), 7.23 (dd, J = 8.4, 5.4 Hz, 1H), 7.01 (t, J = 8.8 Hz, 1H), 4.17 (t, J = 6.7 Hz, 2H), 3.94 (s, 2H), 3.25 (t, J = 5.7 Hz, 4H), 2.04-2.40 (m, 2H), 1.91 (s, 4H). |
| 18 | 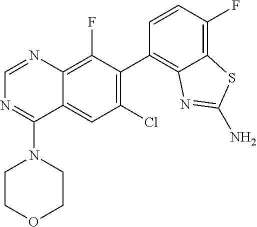 [SMILES: Nc1nc2c(-c3c(Cl)cc4c(N5CCOCC5)ncnc4c3F)ccc(F)c2s1] | LCMS m/z [M + 1]: 434.1 ¹HNMR (400 MHz, CD₃OD): δ 8.66 (s, 1H), 7.99 (d, J = 1.6 Hz, 1H), 7.26 (dd, J = 8.4, 5.4 Hz, 1H), 7.02 (t, J = 8.8 Hz, 1H), 3.95-4.05 (m, 4H), 3.85-3.93 (m, 4H) |
| 19 | 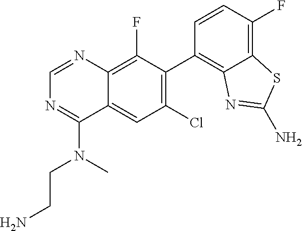 [SMILES: CN(CCN)c1ncnc2c(F)c(-c3ccc(F)c4sc(N)nc34)c(Cl)cc12] | LCMS m/z [M + 1]: 421.2 ¹HNMR (400 MHz, CD₃OD): δ 8.76 (s, 1H), 8.32 (s, 1H), 7.25 (s, 1H), 7.09 (t, J = 8.4 Hz, 1H), 2.65 (s, 3H), 2.51-2.55 (m, 4H) |
| 20 | 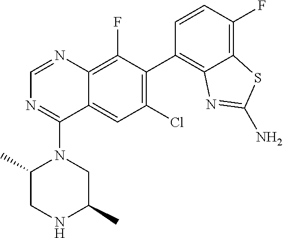 [SMILES: C[C@@H]1CN(c2ncnc3c(F)c(-c4ccc(F)c5sc(N)nc45)c(Cl)cc23)[C@@H](C)CN1] | LCMS m/z [M + 1]: 461.3 ¹HNMR (400 MHz, CD₃OD) 8: 8.87 (s, 1H), 8.09 (s, 1H), 7.22-7.28 (m, 1H), 7.01 (t, J = 8.8 Hz, 1H), 4.56 (s, 3H), 4.26 (s, 1H), 3.83- 3.87 (m, 1H), v3.42-3.56 (m, 3H), 3.03-3.14 (m, 1H), 1.34-1.63 (m, 3H), 1.2-1.28 (m, 3H). |
| 20a | 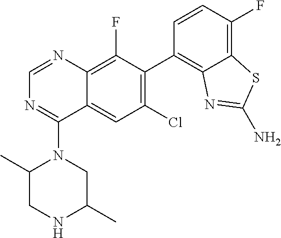 [SMILES: CC1CN(c2ncnc3c(F)c(-c4ccc(F)c5sc(N)nc45)c(Cl)cc23)C(C)CN1] | |
| 21 | 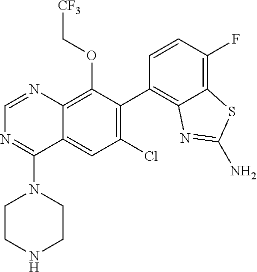 [SMILES: Nc1nc2c(-c3c(Cl)cc4c(N5CCNCC5)ncnc4c3OCC(F)(F)F)ccc(F)c2s1] | LCMS m/z [M + 1]: 513.1 ¹HNMR (400 MHz, CD₃OD): δ 8.75 (s, 1H), 7.97 (s, 1H), 7.15 (dd, J = 8.4, 5.60 Hz, 1H), 6.96 (t, J = 9.20 Hz, 1H), 4.96- 5.01 (m, 1H), 4.58-4.63 (m, 1H), 3.92- 4.08 (m, 4H), 3.31-3.42 (m, 4H). |
| 22 | 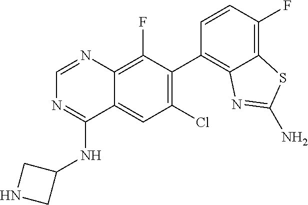 [SMILES: Nc1nc2c(-c3c(Cl)cc4c(NC5CNC5)ncnc4c3F)ccc(F)c2s1] | LCMS m/z [M + 1]: 419.1 ¹HNMR (400 MHz, CD₃OD): δ 8.59 (s, 1H), 8.28 (s, 1H), 7.26 (t, J = 8.4 Hz, 1H), 7.02 (t, J = 8.8 Hz 1H), 5.10-5.13 (m, 4H), 4.31-4.38 (m, 1H). |
| 23 | 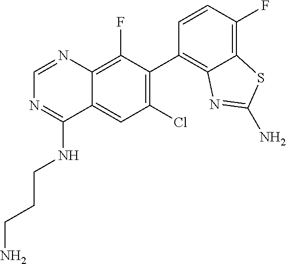 [SMILES: NCCCNc1ncnc2c(F)c(-c3ccc(F)c4sc(N)nc34)c(Cl)cc12] | LCMS m/z [M + 1]: 421.1 ¹HNMR (400 MHz, CD₃OD) δ: 8.73 (s, 1H), 8.36 (s, 1H), 7.27 (dd, J = 8.4, 5.4 Hz, 1H), 7.04 (t, J = 8.8 Hz, 1H), 3.92 (t, J = 6.8 Hz, 2H), 3.10 (t, J = 7.5 Hz, 2H), 2.05-2.20 (m, 2H). |
| 24 | 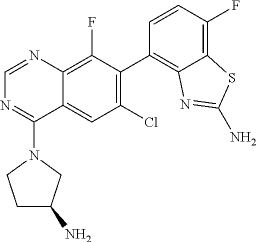 [SMILES: Nc1nc2c(-c3c(Cl)cc4c(N5CC[C@H](N)C5)ncnc4c3F)ccc(F)c2s1] | LCMS m/z [M + 1]: 433.1 ¹HNMR (400 MHz, CD₃OD): δ 8.73 (s, 1H), 8.36 (s, 1H), 7.28 (dd, J = 8.4, 5.6 Hz, 1H), 7.04 (t, J = 8.8 Hz 1H), 4.39-4.50 (m, 3H), 4.20-4.32 (m, 2H), 2.61-2.66 (m, 1H), 2.32- 2.45 (m, 2H). |
| 24a | 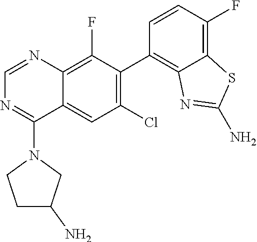 [SMILES: Nc1nc2c(-c3c(Cl)cc4c(N5CCC(N)C5)ncnc4c3F)ccc(F)c2s1] | |
| 25 | 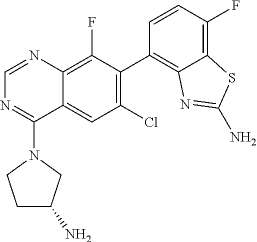 [SMILES: Nc1nc2c(-c3c(Cl)cc4c(N5CC[C@@H](N)C5)ncnc4c3F)ccc(F)c2s1] | LCMS m/z [M + 1]: 433.1 ¹HNMR (400 MHz, CD₃OD): δ 8.74 (s, 1H), 8.37 (s, 1H), 7.29 (dd, J = 8.4, 5.4 Hz, 1H), 7.05 (t, J = 8.8 Hz, 1H), 4.20-4.52 (m, 5H), 2.61-2.73 (m, 1H), 2.41-2.43 (m, 1H). |
| 26 | 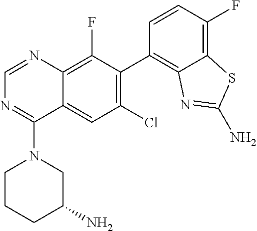 [SMILES: Nc1nc2c(-c3c(Cl)cc4c(N5CCC[C@@H](N)C5)ncnc4c3F)ccc(F)c2s1] | LCMS m/z [M + 1]: 447.1 ¹HNMR (400 MHz, CD₃OD): δ 8.67(s, 1H), 8.00(s, 1H), 7.23-7.27 (m, 1H), 7.01 (t, J = 8.8 Hz, 1H ), 4.33-4.38 (m, 1H), 4.14-4.18 (m, 1H), 3.55-3.65 (m, 2H), 3.48-3.52 (m, 1H), 2.20-2.22 (m, 1H), 1.96-2.00 (m, 1H), 1.79-1.96 (m, 2H). |
| 26a | 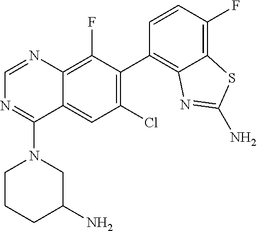 [SMILES: Nc1nc2c(-c3c(Cl)cc4c(N5CCCC(N)C5)ncnc4c3F)ccc(F)c2s1] | |
| 27 | 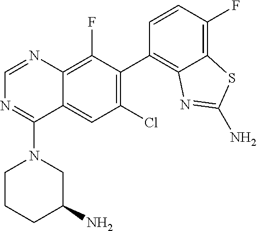 [SMILES: Nc1nc2c(-c3c(Cl)cc4c(N5CCC[C@H](N)C5)ncnc4c3F)ccc(F)c2s1] | LCMS m/z [M + 1]: 447.1 ¹HNMR (400 MHz, CD₃OD): δ 8.73 (s, 1H), 8.09 (s, 1H), 7.27 (t, J = 6.8 Hz, 1H), 7.04 (t, J = 8.8 Hz 1H), 4.52-4.57 (m, 2H), 4.28-4.33 (m, 1H), 3.86-3.91 (m, 2H), 3.61-3.69 (m, 2H), 2.21-2.31 (m, 1H), 1.87-2.03 (m, 3H). |
| 28 | 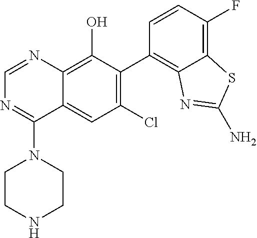 [SMILES: Nc1nc2c(-c3c(Cl)cc4c(N5CCNCC5)ncnc4c3O)ccc(F)c2s1] | LCMS m/z [M + 1]: 431.1 ¹HNMR (400 MHz, CD₃OD): δ 8.75 (s, 1H), 7.83 (s, 1H), 7.25-7.29 (m, 1H), 7.08 (t, J = 8.8 Hz, 1H), 4.44 (t, J = 4.8 Hz, 4H), 3.52 (t, J = 4.8 Hz, 4H). |
| 29 | 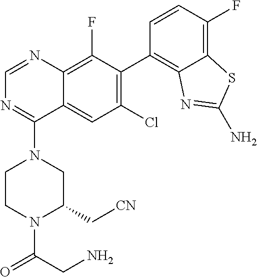 [SMILES: N#CC[C@H]1CN(c2ncnc3c(F)c(-c4ccc(F)c5sc(N)nc45)c(Cl)cc23)CCN1C(=O)CN] | LCMS m/z [M − 1]: 527.0 ¹HNMR (400 MHz, CD₃OD): δ 8.69 (s, 1H), 8.06 (s, 1H), 7.24 (s, 1H), 7.0 (t, J = 8.8 Hz, 1H), 5.10-5.19 (m, 2H), 4.42-4.56 (m, 2H), 3.75-3.85 (m, 2H), 3.69-3.75 (m, 2H), 2.95- 3.10(m, 2H) |
| 29a | 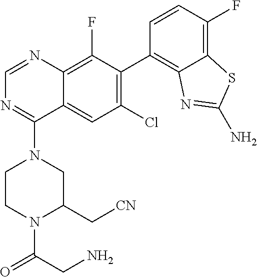 [SMILES: N#CCC1CN(c2ncnc3c(F)c(-c4ccc(F)c5sc(N)nc45)c(Cl)cc23)CCN1C(=O)CN] | |
| 30 | 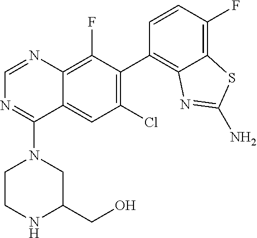 [SMILES: Nc1nc2c(-c3c(Cl)cc4c(N5CCNC(CO)C5)ncnc4c3F)ccc(F)c2s1] | LCMS m/z [M + 1]: 463.1 ¹HNMR (400 MHz, CD₃OD): δ 8.70 (s, 1H), 8.02 (s, 1H), 7.23-7.26 (m, 1H), 7.01 (t, J = 8.8 Hz, 1H), 4.51-4.62 (s, 1H), 4.44-4.51 (m, 2H), 3.66-3.78 (m, 2H), 3.55-3.63 (m, 1H), 3.36-3.48 (m, 3H). |
| 31 | 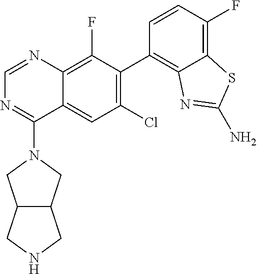 [SMILES: Nc1nc2c(-c3c(Cl)cc4c(N5CC6CNCC6C5)ncnc4c3F)ccc(F)c2s1] | LCMS m/z [M + 1]: 459.1 ¹HNMR (400 MHz, CD₃OD): δ 8.52 (s, 1H), 8.25 (s, 1H), 7.23 (dd, J = 8.3, 5.5 Hz, 1H), 7.01 (t, J = 8.8 Hz, 1H), 4.60 (s, 4H), 4.26- 4.39 (m, 2H), 4.06-4.17 (m, 2H), 3.53-3.68 (m, 2H). |
| 32 | 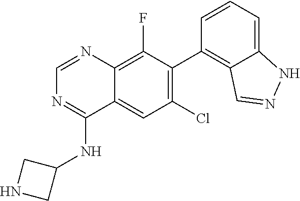 [SMILES: Fc1c(-c2cccc3[nH]ncc23)c(Cl)cc2c(NC3CNC3)ncnc12] | LCMS m/z [M + 1]: 369.1 ¹H NMR (400 MHz, CD₃OD) δ 8.69 (s, 1H), 8.38 (s, 1H), 7.72 (d, J = 7.9 Hz, 2H), 7.57 (t, J = 7.7 Hz, 1H), 7.21 (d, J = 6.9 Hz, 1H), 5.21-5.08 (m, 1H), 4.59-4.38 (m, 4H). |
| 33 | 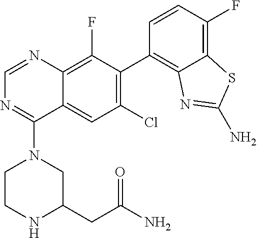 [SMILES: NC(=O)CC1CN(c2ncnc3c(F)c(-c4ccc(F)c5sc(N)nc45)c(Cl)cc23)CCN1] | LCMS m/z [M + 1]: 490.1 ¹HNMR (400 MHz, CD₃OD) δ 8.69 (s, 1H), 8.01 (s, 1H), 7.21-7.30 (m, 1H), 7.00 (t, J = 8.8 Hz, 1H), 5.18-5.30 (m, 2H) 4.20-4.28 (m, 2H), 3.50-3.70 (m, 6H), 2.52-2.60 (m, 1H). |
| 36 | 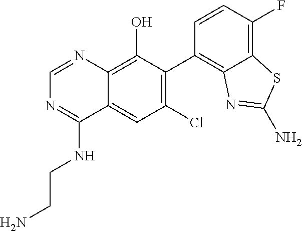 [SMILES: NCCNc1ncnc2c(O)c(-c3ccc(F)c4sc(N)nc34)c(Cl)cc12] | LCMS m/z [M + 1]: 405.1 ¹HNMR (400 MHz, CD₃OD): δ: 8.73 (s, 1H), 8.01 (s, 1H), 7.20-7.24 (dd, J = 8.2, 5.6 Hz, 1H), 7.02-7.06 (t, J = 8.2 Hz, 1H), 4.09-4.19 (m, 2H), 3.61-3.90 (m, 2H) |
| 37 | 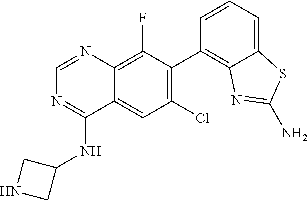 [SMILES: Nc1nc2c(-c3c(Cl)cc4c(NC5CNC5)ncnc4c3F)cccc2s1] | LCMS m/z [M + 1]: 401.01 ¹HINMR (400 MHz, CD₃OD): δ 8.63 (s, 1H), 8.27 (s, 1H), 7.77 (d, J = 8.8 Hz, 1H), 6.97-7.46 (m, 3H), 5.10 (d, J = 7.6 Hz, 1H), 4.47 (dt, J = 18.5, 11.0 Hz, 3H). |
| 38 | 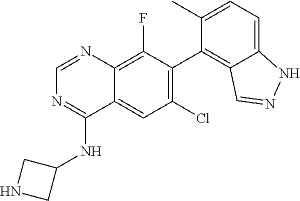 [SMILES: Cc1ccc2[nH]ncc2c1-c1c(Cl)cc2c(NC3CNC3)ncnc2c1F] | LCMS m/z [M + 1]: 383.1 ¹HNMR (400 MHz, CD₃OD): δ 8.66 (s, 1H), 8.36 (s, 1H), 8.14 (s, 1H), 7.61 (d, J = 8.5 Hz, 1H), 7.40-7.55 (m, 2H), 5.01-5.31 (m, 2H), 4.48 (dt, J = 18.5, 11.0 Hz, 4H), 2.22 (s, 3H). |
| 40 | 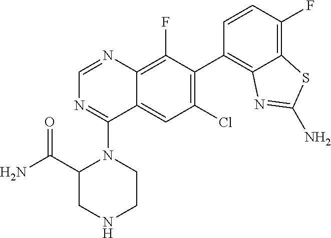 [SMILES: NC(=O)C1CNCCN1c1ncnc2c(F)c(-c3ccc(F)c4sc(N)nc34)c(Cl)cc12] | LCMS m/z [M + 1]: 476.1 ¹HNMR (400 MHz, CD₃OD): δ8.16 (s, 1H), 7.71 (s, 1H), 7.21-7.24 (m, 1H), 7.01 (d, J = 8.8 Hz), 4.35-4.5 (m, 4H), 3.70-3.80 (m, 2H). |
| 41 | 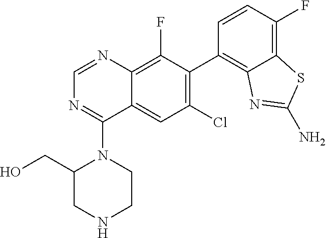 [SMILES: Nc1nc2c(-c3c(Cl)cc4c(N5CCNCC5CO)ncnc4c3F)ccc(F)c2s1] | LCMS m/z [M + 1]: 463.1 ¹HNMR (400 MHz, CD₃OD): δ 8.82 (s, 1H), 8.35 (s, 1H), 7.23-7.30 (m, 1H), 7.02 (t, J = 8.8 Hz, 1H), 4.50-4.79 (m, 2H), 3.41-3.55 (m, 2H), 3.20-3.30 (m, 2H), 2.88-3.11 (m, 3H). |
| 42 | 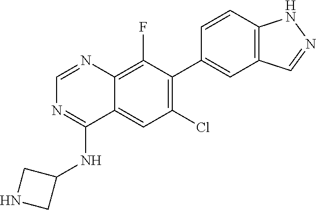 [SMILES: Fc1c(-c2ccc3[nH]ncc3c2)c(Cl)cc2c(NC3CNC3)ncnc12] | LCMS m/z [M + 1]: 369.1 ¹HNMR (400 MHz, CD₃OD): δ 8.62 (s, 1H), 8.30 (s, 1H), 8.07 (s, 1H), 7.79 (s, 1H), 7.61 (t, J = 8.4 Hz, 1H), 7.34 (t, J = 8.4 Hz, 1H), 5.09 (t, J = 8.4 Hz, 2H), 4.39 (m, 4H). |
| 43 | 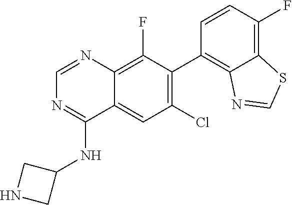 [SMILES: Fc1c(-c2ccc(F)c3scnc23)c(Cl)cc2c(NC3CNC3)ncnc12] | LCMS m/z [M + 1]: 404.1 ¹HNMR (400 MHz, CD3OD): δ 9.2 (s, 1H), 8.67 (s, 1H), 8.34 (s, 1H), 7.61-7.63 (m, 1H), 7.47-7.49 (m, 1H), 5.10-5.14 (m, 1H), 4.46- 4.50 (m, 4H). |
| 50 | 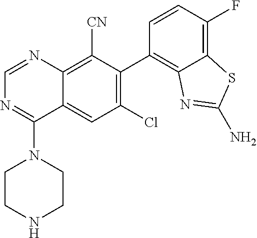 [SMILES: N#Cc1c(-c2ccc(F)c3sc(N)nc23)c(Cl)cc2c(N3CCNCC3)ncnc12] | LCMS m/z [M + 1]: 440.1 ¹HNMR (400 MHz, CD₃OD): δ 8.83 (s, 1H), 8.45(s, 1H), 7.28-7.32 (dd, J = 8.2, 5.2 Hz, 1H), 7.03-7.07 (t, J = 8.8 Hz, 1H), 4.12-4.14 (m, 4H), 3.46-3.49 (m, 4H). |
| 58 | 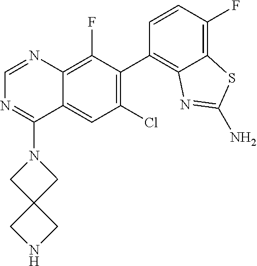 [SMILES: Nc1nc2c(-c3c(Cl)cc4c(N5CC6(CNC6)C5)ncnc4c3F)ccc(F)c2s1] | LCMS m/z [M + 1]: 445.0 ¹HNMR (700 MHz, CD₃OD) δ 8.46 (s, 1H), 7.89 (d, J = 1.6 Hz, 1H), 7.23 (dd, J = 8.4, 5.4 Hz, 1H), 7.00 (dd, J = 9.1, 8.4 Hz, 1H), 4.79 (s, 5H), 4.14 (s, 4H). |
| 65 | 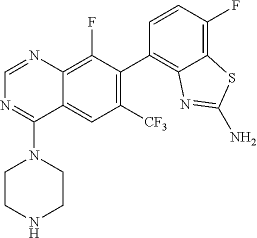 [SMILES: Nc1nc2c(-c3c(C(F)(F)F)cc4c(N5CCNCC5)ncnc4c3F)ccc(F)c2s1] | LCMS m/z (M + 1) 467.4 ¹HNMR (300 MHz, CD₃OD) δ 8.89 (s, 1H), 8.33 (s, 1H), 7.33-7.26 (m, 1H), 7.07 (t, J = 8.8 Hz, 1H), 4.37-4.30 (m, 4H), 3.58-3.47 (m, 4H). |
| 66 | 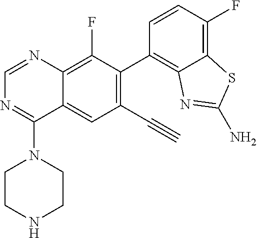 [SMILES: C#Cc1cc2c(N3CCNCC3)ncnc2c(F)c1-c1ccc(F)c2sc(N)nc12] | LCMS m/z (M + 1) 423.2 ¹HNMR (300 MHz, CD₃OD) δ 8.75 (s, 1H), 8.09 (s, 1H), 7.30 (dd, J = 8.5, 5.4 Hz, 1H), 6.99 (t, J = 8.8 Hz, 1H), 4.25-4.07 (m, 4H), 3.57 (s, 1H), 3.54-3.40 (m, 4H). |
| 88 | 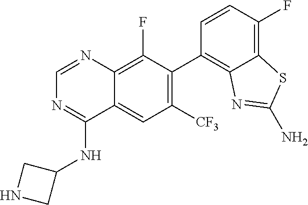 [SMILES: Nc1nc2c(-c3c(C(F)(F)F)cc4c(NC5CNC5)ncnc4c3F)ccc(F)c2s1] | LCMS m/z [M + 1]: 453.1 ¹H NMR (300 MHz, CD₃OD) δ 8.75 (s, 1H), 8.64 (s, 1H), 7.29-7.21 (m, 1H), 7.02 (t, J = 8.9 Hz, 1H), 5.21-5.13 (m, 1H), 4.58-4.42 (m, 4H). |
| 94 | 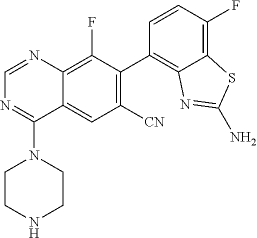 [SMILES: N#Cc1cc2c(N3CCNCC3)ncnc2c(F)c1-c1ccc(F)c2sc(N)nc12] | LCMS m/z [M + 1]: 424.2 ¹H NMR (300 MHz, CD₃OD) δ 8.80 (s, 1H), 8.41 (s, 1H), 7.38 (dd, J = 8.5 and 5.4 Hz, 1H), 7.05 (t, J = 8.8 Hz, 1H), 4.26-4.05 (m, 4H), 3.51-3.36 (m, 4H). |
| 110 | 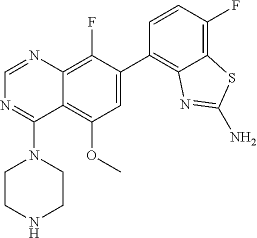 [SMILES: COc1cc(-c2ccc(F)c3sc(N)nc23)c(F)c2ncnc(N3CCNCC3)c12] | |
| 111 | 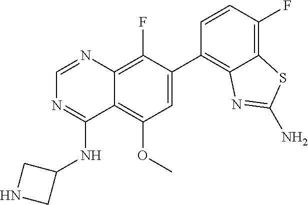 [SMILES: COc1cc(-c2ccc(F)c3sc(N)nc23)c(F)c2ncnc(NC3CNC3)c12] | |
| 117 | 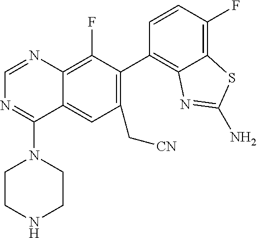 [SMILES: N#CCc1cc2c(N3CCNCC3)ncnc2c(F)c1-c1ccc(F)c2sc(N)nc12] | LCMS m/z [M + 1]: 424 ¹HNMR (300 MHz, CD₃OD) δ 8.80 (s, 1H), 8.41 (s, 1H), 7.38 (dd, J = 8.5, 5.4 Hz, 1H), 7.05 (t, J = 8.8 Hz, 1H), 4.26-4.05 (m, 4H), 3.51-3.36 (m, 4H). |

Additional compounds of the present disclosure are included in Table 3.

TABLE 3
Selected compounds of the present disclosure.

| Compound | | |
|---|---|---|
| No. | Structure | Analytical data |
| 49 | 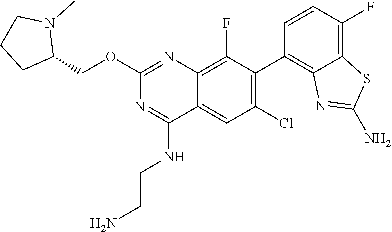 [SMILES: CN1CCC[C@H]1COc1nc(NCCN)c2cc(Cl)c(-c3ccc(F)c4sc(N)nc34)c(F)c2n1] | LCMS m/z [M + 1]: 520.2 ¹HNMR (400 MHz, CD₃OD): δ 8.16 (s, 1H), 7.22-7.26 (m, 1H), 7.03 (t, J = 8.4 Hz, 1H), 4.90-4.95 (m, 1H), 4.66-4.70 (m, 1H), 3.80-4.10 (m, 3H), 3.68-3.80 (m, 1H), 3.35-3.42 (m, 2H), 3.20-3.28 (m, 1H), 3.09 (s, 3H), 1.35-1.45 (m, 1H), 2.02-2.30 (m, 3H). |
| 49a | 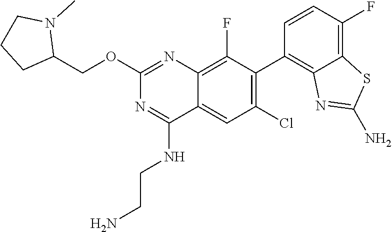 [SMILES: CN1CCCC1COc1nc(NCCN)c2cc(Cl)c(-c3ccc(F)c4sc(N)nc34)c(F)c2n1] | |
| 53 | 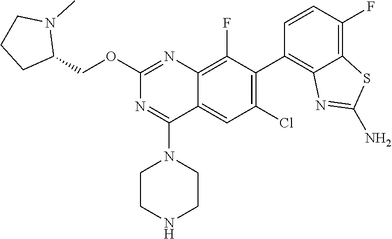 [SMILES: CN1CCC[C@H]1COc1nc(N2CCNCC2)c2cc(Cl)c(-c3ccc(F)c4sc(N)nc34)c(F)c2n1] | LCMS m/z [M + 1]: 546.2 ¹HNMR (400 MHz, CD₃OD): δ 7.99 (s, 1H) 7.20-7.22 (m, 1H) 7.01 (t, J = 8.8 Hz, 1H), 4.64-4.68 (m, 1H), 4.15 (s, 4H), 3.89 (s, 1H), 3.72 (s, 1H) 3.59 (s, 4H), 3.09 (s, 3H), 2.39-2.42 (m, 1H), 2.19- 2.21 (m,1H), 2.03-2.08 (m,2H), 1.28 (s, 1H). |
| 53a | 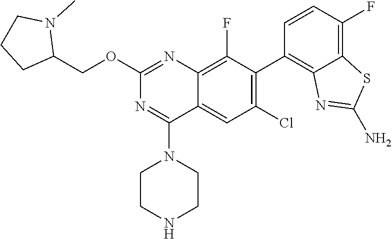 [SMILES: CN1CCCC1COc1nc(N2CCNCC2)c2cc(Cl)c(-c3ccc(F)c4sc(N)nc34)c(F)c2n1] | |
| 54 | 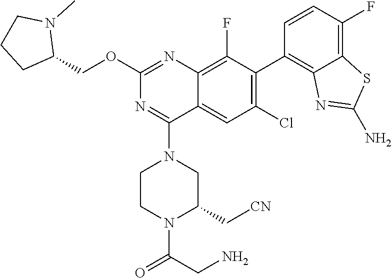 [SMILES: CN1CCC[C@H]1COc1nc(N2CCN(C(=O)CN)[C@@H](CC#N)C2)c2cc(Cl)c(-c3ccc(F)c4sc(N)nc34)c(F)c2n1] | LCMS m/z [M + 1]: 642.2 ¹HINMR (400 MHz, CD₃OD): δ 8.04 (s, 1H), 7.21-7.24 (m, 1H), 7.02 (t, J = 8.4 Hz, 1H), 5.03(s, 1H), 4.90-4.92 (m, 1H), 4.49-4.68 (m, 3H), 4.13-3.98 (m, 2H), 3.75-3.87 (m, 5H), 3.21-3.25 (m, 2H), 2.97-3.09 (m, 5H), 2.37-2.49 (m, 1H), 2.03-2.23 (m, 3H). |
| 54a | 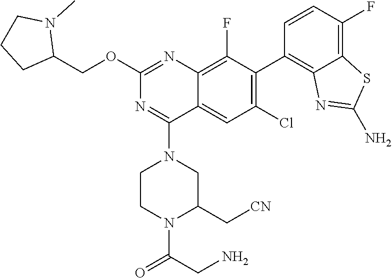 [SMILES: CN1CCCC1COc1nc(N2CCN(C(=O)CN)C(CC#N)C2)c2cc(Cl)c(-c3ccc(F)c4sc(N)nc34)c(F)c2n1] | |
| 55 | 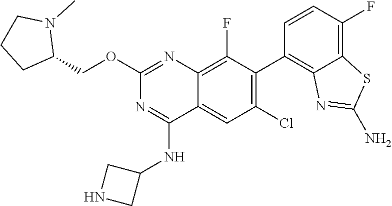 [SMILES: CN1CCC[C@H]1COc1nc(NC2CNC2)c2cc(Cl)c(-c3ccc(F)c4sc(N)nc34)c(F)c2n1] | LCMS m/z [M + 1]: 532.1 ¹HNMR (400 MHz, CD₃OD): δ 8.21 (s, 1H), 7.19-7.23 (m, 1H), 7.21-7.24 (m, 1H), 8.80 (t, J = 8.8 Hz, 1H), 5.15-5.18 (m, 1H), 4.77-4.80 (m, 1H), 4.58-4.63 (m, 1H), 4.44-4.48 (m, 2H), 4.34-4.36 (m, 2H), 3.59-3.68 (m, 2H), 3.08 (m, 1H), 2.98 (s, 3H), 2.33-2.35 (m, 1H), 2.02-2.12 (m, 3H). |
| 55a | 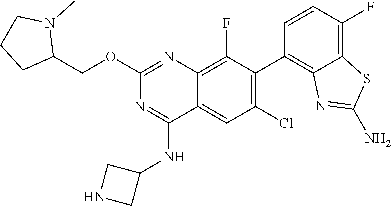 [SMILES: CN1CCCC1COc1nc(NC2CNC2)c2cc(Cl)c(-c3ccc(F)c4sc(N)nc34)c(F)c2n1] | |
| 56 | 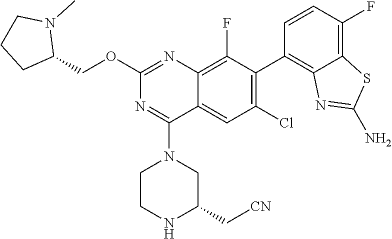 [SMILES: CN1CCC[C@H]1COc1nc(N2CCN[C@@H](CC#N)C2)c2cc(Cl)c(-c3ccc(F)c4sc(N)nc34)c(F)c2n1] | LCMS m/z [M + 1]: 585.2 ¹HNMR (400 MHz, CD₃OD): δ 8.02 (s, 1H), 7.23-7.20 (m, 1H), 7.02 (t, J = 8.8 Hz, 1H), 4.62-4.72 (m, 2H), 4.51 (d, J = 7.2 Hz, 1H), 4.04-4.06 (m, 1H), 3.81- 3.91 (m, 2H), 3.75-3.57 (m,3H), 3.46- 3.49 (m, 1H), 3.22-3.26 (m, 1H), 3.12- 3.10 (m,5H), 2.39-2.42 (m, 1H), 2.19- 2.21 (m, 1H), 2.07-2.09 (m, 2H). |
| 56a | 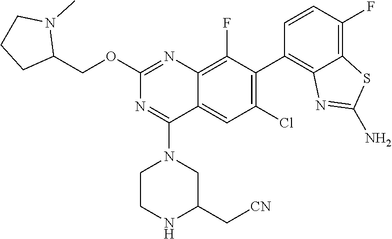 [SMILES: CN1CCCC1COc1nc(N2CCNC(CC#N)C2)c2cc(Cl)c(-c3ccc(F)c4sc(N)nc34)c(F)c2n1] | |
| 57 | 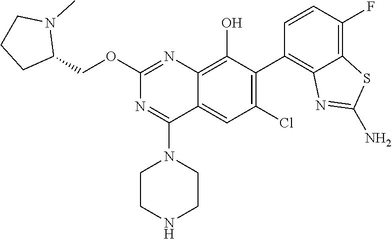 [SMILES: CN1CCC[C@H]1COc1nc(N2CCNCC2)c2cc(Cl)c(-c3ccc(F)c4sc(N)nc34)c(O)c2n1] | LCMS m/z [M + 1]: 544.2 ¹HNMR (400 MHz, CD₃OD): δ 8.47 (s, 2H), 7.58 (s, 1H), 7.16 (dd, J = 8.3, 5.5 Hz, 1H), 6.98 (t, J = 8.8 Hz, 1H), 4.80 (d, J = 3.0 Hz, 1H), 4.62 (ddd, J = 13.1, 6.4, 2.5 Hz, 2H), 3.92-4.05 (m, 5H), 3.55-3.64 (m, 2H), 2.90-3.07 (m, 5H), 2.32 (dd, J = 14.1, 8.0 Hz, 1H), 1.96- 2.14 (m, 4H). |
| 57a | 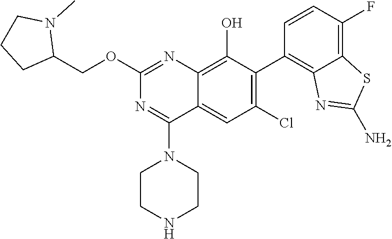 [SMILES: CN1CCCC1COc1nc(N2CCNCC2)c2cc(Cl)c(-c3ccc(F)c4sc(N)nc34)c(O)c2n1] | |
| 59 | 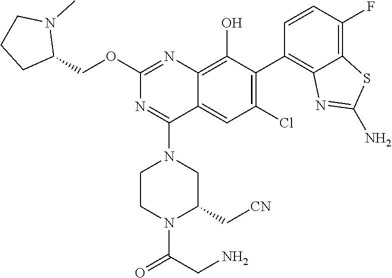 [SMILES: CN1CCC[C@H]1COc1nc(N2CCN(C(=O)CN)[C@@H](CC#N)C2)c2cc(Cl)c(-c3ccc(F)c4sc(N)nc34)c(O)c2n1] | LCMS m/z [M + 1]: 640.3 ¹HNMR (400 MHz, CD₃OD) δ 8.48 (s, 2H), 7.61-7.65 (m, 1H), 7.12-7.19 (m, 1H), 6.94-7.02 (m, 1H), 5.01-5.09 (m, 1H), 4.72-4.79 (m, 1H), 4.60 (dd, J = 11.9, 7.0 Hz, 1H), 4.44 (d, J = 11.5 Hz, 2H), 3.89-4.03 (m, 2H), 3.70-3.82 (m, 2H), 3.60-3.67 (m, 1H), 3.42-3.58 (m, 3H), 3.37-3.42 (m, 1H), 2.81-3.08 (m, 5H), 2.25-2.32 (m, 1H), 1.92-2.11 (m, 3H). |
| 59a | 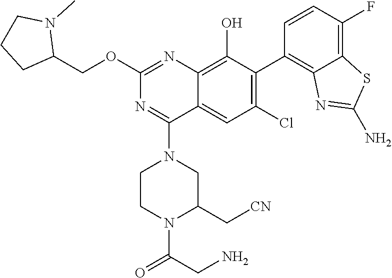 [SMILES: CN1CCCC1COc1nc(N2CCN(C(=O)CN)C(CC#N)C2)c2cc(Cl)c(-c3ccc(F)c4sc(N)nc34)c(O)c2n1] | |
| 60 | 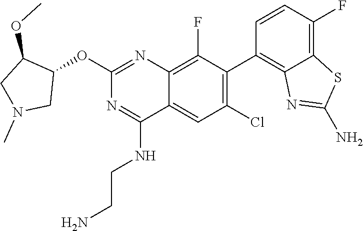 [SMILES: CO[C@@H]1CN(C)C[C@H]1Oc1nc(NCCN)c2cc(Cl)c(-c3ccc(F)c4sc(N)nc34)c(F)c2n1] | LCMS m/z [M + 1]: 536.2 ¹HINMR (400 MHz, CD₃OD): δ: 8.11 (s, 1H), 7.19-7.21 (m, 1H), 7.00 (t, J = 8.6 Hz, 1H), 5.71-5.75 (m, 1H), 4.33 (s, 3H), 3.57 (d, J = 2.8 Hz, 3H), 3.30-3.55 (m, 6H), 3.06 (s, 3H). |
| 60a | 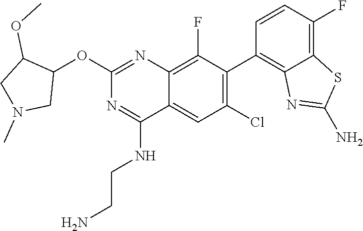 [SMILES: COC1CN(C)CC1Oc1nc(NCCN)c2cc(Cl)c(-c3ccc(F)c4sc(N)nc34)c(F)c2n1] | |
| 61 | 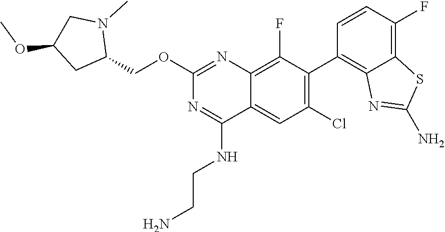 [SMILES: CO[C@@H]1C[C@@H](COc2nc(NCCN)c3cc(Cl)c(-c4ccc(F)c5sc(N)nc45)c(F)c3n2)N(C)C1] | LCMS m/z [M + 1]: 550.2 ¹HNMR (400 MHz, CD₃OD) δ 8.14 (s, 1H), 7.19-7.25 (m, 1H), 6.98-7.04 (t, J = 8.8 Hz, 1H), 4.90-5.00 (m, 2H), 4.60- 4.75 (m, 1H), 3.76-4.20 (m, 5H), 3.38 (s, 3H), 3.12 (s, 3H), 2.51 (dd, J = 14.3, 6.4 Hz, 2H), 2.05-2.20 (m, 2H). |
| 61a | 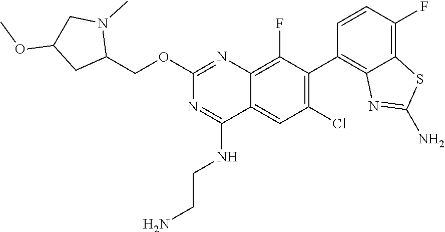 [SMILES: COC1CC(COc2nc(NCCN)c3cc(Cl)c(-c4ccc(F)c5sc(N)nc45)c(F)c3n2)N(C)C1] | |
| 62 | 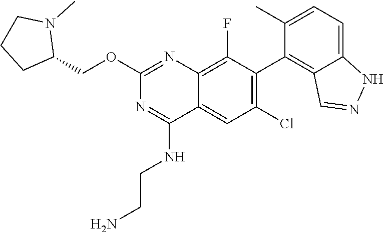 [SMILES: Cc1ccc2[nH]ncc2c1-c1c(Cl)cc2c(NCCN)nc(OC[C@@H]3CCCN3C)nc2c1F] | LCMS m/z [M + 1]: 484.2 ¹HNMR(400 MHz, CD₃OD): δ 8.23 (s, 1H), 7.59 (d, J = 8.4 Hz, 1H), 7.42-7.48 (m, 2H), 4.95-5.00 (m, 1H), 4.62-4.72 (m, 1H), 3.98 (t, J = 6.0 Hz, 2H), 3.85- 3.92 (m, 1H), 3.65-3.78 (m, 1H), 3.33- 3.40 (m, 3H), 3.09 (s, 3H), 2.30-2.42 (m, 1H), 2.21 (s, 3H), 2.02-2.15 (m, 3H). |
| 62a | 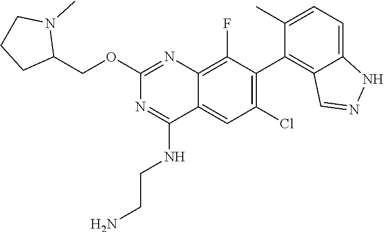 [SMILES: Cc1ccc2[nH]ncc2c1-c1c(Cl)cc2c(NCCN)nc(OCC3CCCN3C)nc2c1F] | |
| 63 | 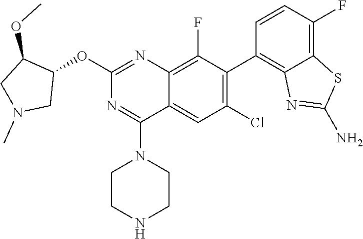 [SMILES: CO[C@@H]1CN(C)C[C@H]1Oc1nc(N2CCNCC2)c2cc(Cl)c(-c3ccc(F)c4sc(N)nc34)c(F)c2n1] | LCMS m/z [M + 1]: 562.1 ¹HNMR (400 MHz, CD₃OD): δ 7.98 (s, 1H), 7.24(dd, J = 8.4, 5.2 Hz, 1H), 7.04 (t, J = 8.8 Hz, 1H), 4.34 (s, 1H), 4.09 (t, J = 4.4 Hz, 6H), 3.57 (d, J = 1.2 Hz, 4H), 3.49(t, J = 4.8 Hz, 6H), 3.06 (s, 3H). |
| 63a | 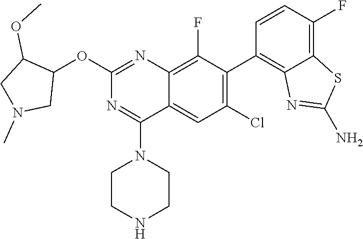 [SMILES: COC1CN(C)CC1Oc1nc(N2CCNCC2)c2cc(Cl)c(-c3ccc(F)c4sc(N)nc34)c(F)c2n1] | |
| 64 | 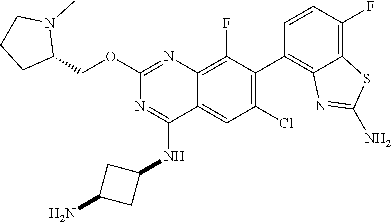 | LCMS m/z [M + 1]: 547.1 ¹HNMR (400 MHz, CD3OD): δ 8.27 (s, 1H), 7.21-7.24 (m, 1H), 7.01 (s, 1H), 4.63-4.67 (m, 1H), 4.53-4.57 (m, 1H), 3.80-3.86 (m, 2H), 3.69-3.66 (m, 3H), 3.08 (s, 1H), 2.30-2.97 (m, 3H), 2.33- 2.40 (m, 4H). |
| 64a | 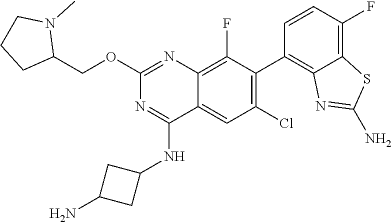 [SMILES: CN1CCCC1COc1nc(NC2CC(N)C2)c2cc(Cl)c(-c3ccc(F)c4sc(N)nc34)c(F)c2n1] | |
| 67 | 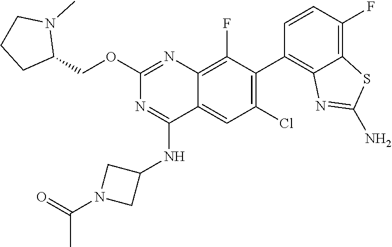 [SMILES: CC(=O)N1CC(Nc2nc(OC[C@@H]3CCCN3C)nc3c(F)c(-c4ccc(F)c5sc(N)nc45)c(Cl)cc23)C1] | LCMS m/z [M + 1]: 574.2 ¹HNMR (400 MHz, CD3OD) δ 8.27 (s, 1H), 7.26-7.20 (m, 1H), 7.02 (t, J = 8.9 Hz, 1H), 5.01-4.96 (m, 1H), 4.71- 4.59 (m, 2H), 4.48-4.41 (m, 1H), 4.39- 4.32 (m, 1H), 4.13 (dt, J = 10.4, 5.3 Hz, 1H), 3.84-3.94 (m, 1H), 3.67-3.77 (m, 1H), 3.18-3.28 (m, 2H), 3.08 (s, 3H), 2.37-2.43 (m, 1H), 2.16-2.23 (m, 1H), 2.03-2.13 (m, 2H), 1.94 (s, 3H). |
| 67a | 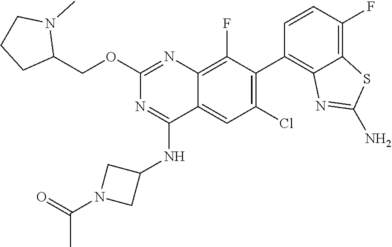 [SMILES: CC(=O)N1CC(Nc2nc(OCC3CCCN3C)nc3c(F)c(-c4ccc(F)c5sc(N)nc45)c(Cl)cc23)C1] | |
| 68 | 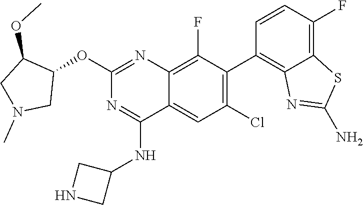 [SMILES: CO[C@@H]1CN(C)C[C@H]1Oc1nc(NC2CNC2)c2cc(Cl)c(-c3ccc(F)c4sc(N)nc34)c(F)c2n1] | LCMS m/z [M + 1]: 548.2 ¹HNMR (400 MHz, CD₃OD): δ 8.50 (s, 2H), 8.17 (s, 1H), 7.18 (d, J = 5.6 Hz, 1H), 6.98-7.00 (m, 1H), 5.51(s, 1H), 5.09 (s, 2H), 4.43 (d, J = 8 Hz, 1H), 4.15 (s, 1H), 3.49 (s, 4H), 2.33-2.40 (m, 4H), 2.64 (s, 3H). |
| 68a | 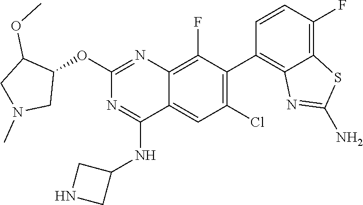 [SMILES: COC1CN(C)C[C@H]1Oc1nc(NC2CNC2)c2cc(Cl)c(-c3ccc(F)c4sc(N)nc34)c(F)c2n1] | |
| 69 | 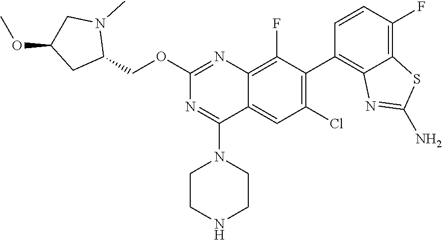 [SMILES: CO[C@@H]1C[C@@H](COc2nc(N3CCNCC3)c3cc(Cl)c(-c4ccc(F)c5sc(N)nc45)c(F)c3n2)N(C)C1] | LCMS m/z [M + 1]: 576.3 ¹H NMR (400 MHz, CD₃OD) δ 7.97 (s, 1H), 7.21-7.23 (m, 1H), 7.01 (t, J = 8.8 Hz, 1H), 4.65 (dd, J = 13.1, 5.9 Hz, 1H), 4.08-4.14 (m, 6H), 3.86-3.95 (m, 1H), 3.76 (d, J = 11.9 Hz, 1H), 3.37- 3.42 (m, 8H), 3.08 (s, 3H), 2.36-2.57 (m, 1H), 2.05-2.20 (m, 1H). |
| 69a | 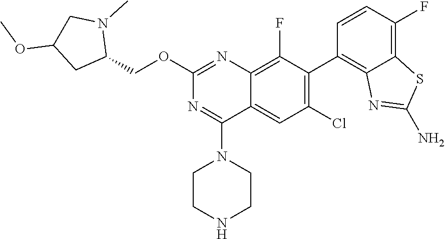 [SMILES: COC1C[C@@H](COc2nc(N3CCNCC3)c3cc(Cl)c(-c4ccc(F)c5sc(N)nc45)c(F)c3n2)N(C)C1] | |
| 70 | 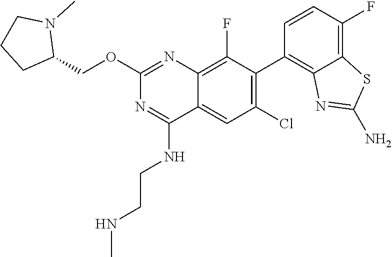 [SMILES: CNCCNc1nc(OC[C@@H]2CCCN2C)nc2c(F)c(-c3ccc(F)c4sc(N)nc34)c(Cl)cc12] | LCMS m/z [M + 1]: 534.2 ¹HNMR (400 MHz, CD₃OD) δ 8.12 (s, 1H), 7.15-7.29 (m, 1H), 7.01 (t, J = 8.8 Hz, 1H), 4.58-4.71 (m, 1H), 3.82- 4.13 (m, 4H), 3.71 (s, 1H), 3.40 (d, J = 4.6 Hz, 2H), 3.08 (s, 2H), 2.80 (s, 2H), 2.32-2.46 (m, 1H), 1.97-2.26 (m, 4H). |
| 70a | 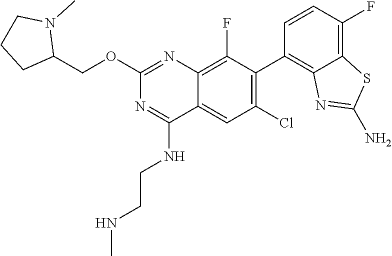 [SMILES: CNCCNc1nc(OCC2CCCN2C)nc2c(F)c(-c3ccc(F)c4sc(N)nc34)c(Cl)cc12] | |
| 71 | 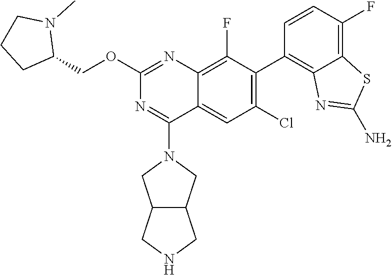 [SMILES: CN1CCC[C@H]1COc1nc(N2CC3CNCC3C2)c2cc(Cl)c(-c3ccc(F)c4sc(N)nc34)c(F)c2n1] | LCMS m/z [M + 1]: 572.2 ¹HNMR (400 MHz, CD₃OD) δ 8.50 (s, 2H), 8.21 (s, 1H), 7.16-7.26 (m, 1H), 7.00 (t, J = 8.8 Hz, 1H), 4.68-4.76 (m, 1H), 4.55-4.62 (m, 1H), 4.27-4.41 (m, 2H), 4.09-4.13 (m, 2H), 3.50-3.61 (m, 4H), 2.93-3.02 (m, 1H), 2.90 (s, 3H), 2.21-2.38 (m, 1H), 1.88-2.10 (m, 2H). |
| 71a | 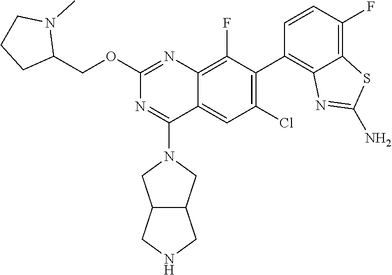 [SMILES: CN1CCCC1COc1nc(N2CC3CNCC3C2)c2cc(Cl)c(-c3ccc(F)c4sc(N)nc34)c(F)c2n1] | |
| 72 | 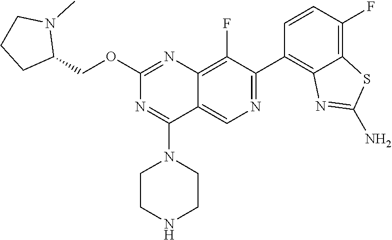 [SMILES: CN1CCC[C@H]1COc1nc(N2CCNCC2)c2cnc(-c3ccc(F)c4sc(N)nc34)c(F)c2n1] | LCMS m/z [M + 1]: 513.3 ¹HNMR (300 MHz, CDCl₃) δ 9.07 (s, 1H), 7.72 (dd, J = 8.6, 5.4 Hz, 1H), 7.15 (t, J = 8.7 Hz, 1H), 4.59 (m, 1H), 4.41 (m, 1H), 3.99 (m, 4H), 3.68 (m, 4H), 3.17 (m, 1H), 2.77 (m, 1H), 2.54 (s, 3H), 2.32 (m, 1H), 2.07 (m, 1H), 1.83 (m, 3H), 1.58 (s, 9H), 1.53 (s, 9H). |
| 72a | 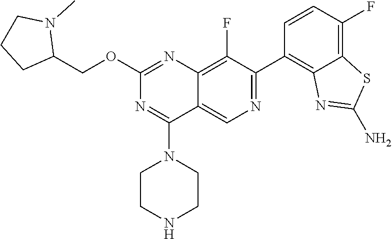 [SMILES: CN1CCCC1COc1nc(N2CCNCC2)c2cnc(-c3ccc(F)c4sc(N)nc34)c(F)c2n1] | |
| 73 | 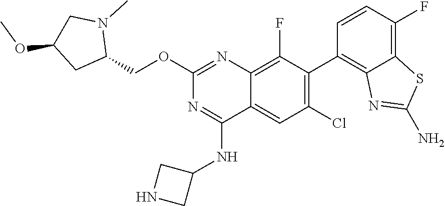 [SMILES: CO[C@@H]1C[C@@H](COc2nc(NC3CNC3)c3cc(Cl)c(-c4ccc(F)c5sc(N)nc45)c(F)c3n2)N(C)C1] | LCMS m/z [M + 1]: 562.2 ¹HNMR(400 MHz, CD₃OD): δ 8.18 (s, 1H), 7.21 (t, J = 6.4 Hz, 1H), 6.99 (d, J = 8.8 Hz, 1H), 5.11-5.21 (m, 1H), 4.25- 4.70 (m, 6H), 4.04-4.10 (m, 1H), 3.41- 3.66 (m, 2H), 3.30 (s, 3H), 2.72-2.86 (m, 4H), 2.20-2.31 (m, 1H), 1.95-2.10 (m, 1H) |
| 73a | 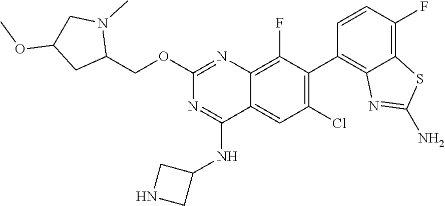 [SMILES: COC1CC(COc2nc(NC3CNC3)c3cc(Cl)c(-c4ccc(F)c5sc(N)nc45)c(F)c3n2)N(C)C1] | |
| 75 | 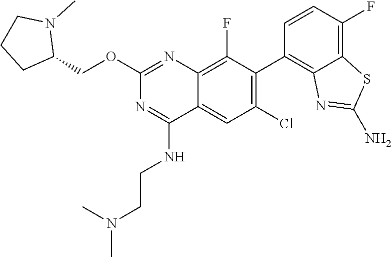 [SMILES: CN(C)CCNc1nc(OC[C@@H]2CCCN2C)nc2c(F)c(-c3ccc(F)c4sc(N)nc34)c(Cl)cc12] | LCMS m/z [M + 1]: 548.2 ¹HNMR(400 MHz, CD₃OD): δ 8.12 (s, 1H), 7.22 (dd, J = 8.4, 5.2 Hz, 1H), 7.03 (dd, J = 8.8, 1.6 Hz, 1H), 4.94-4.97 (m, 1H), 4.62-4.69 (m, 1H), 4.12-4.14 (m, 1H), 3.98-4.00 (m, 1H), 3.88 (s, 1H), 3.72 (s, 1H), 3.51-3.57 (m, 2H), 3.30- 3.31 (m, 1H), 3.07 (s, 3H), 3.03 (s, 6H), 2.20-2.41 (m, 1H), 2.03-2.19 (m, 3H). |
| 75a | 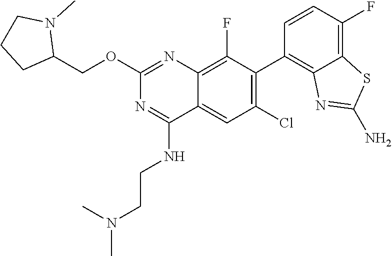 [SMILES: CN(C)CCNc1nc(OCC2CCCN2C)nc2c(F)c(-c3ccc(F)c4sc(N)nc34)c(Cl)cc12] | |
| 76 | 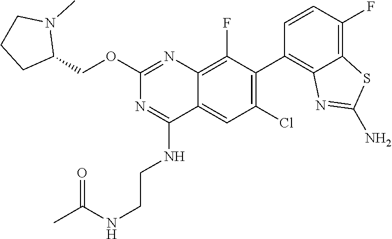 [SMILES: CC(=O)NCCNc1nc(OC[C@@H]2CCCN2C)nc2c(F)c(-c3ccc(F)c4sc(N)nc34)c(Cl)cc12] | LCMS m/z [M + 1]: 562.2 |
| 76a | 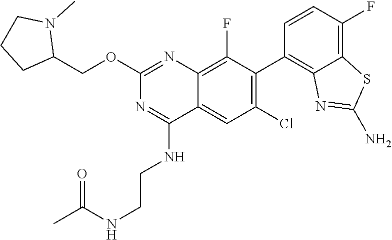 [SMILES: CC(=O)NCCNc1nc(OCC2CCCN2C)nc2c(F)c(-c3ccc(F)c4sc(N)nc34)c(Cl)cc12] | |
| 77 | 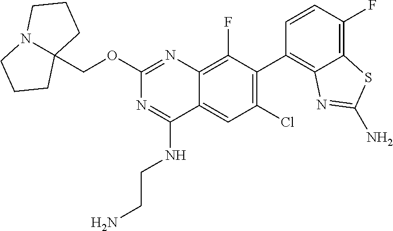 [SMILES: NCCNc1nc(OCC23CCCN2CCC3)nc2c(F)c(-c3ccc(F)c4sc(N)nc34)c(Cl)cc12] | LCMS m/z [M + 1]: 546.2 ¹HNMR (400 MHz, CD³OD) δ 8.16 (s, 1H), 7.23 (dd, J = 8.4, 5.4 Hz, 1H), 7.03 (t, J = 8.8 Hz, 1H), 4.59-4.76 (m, 2H), 3.90-4.06 (m, 2H), 3.78-3.60 (m, 2H), 3.32-3.37 (m, 4H), 2.26-2.38 (m, 2H), 2.17-2.25 (m, 2H), 2.04-2.16 (m, 4H). |
| 78 | 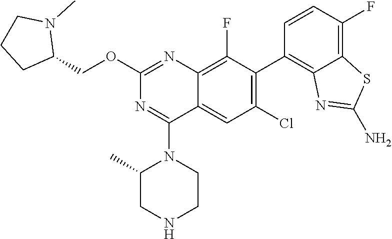 [SMILES: C[C@H]1CNCCN1c1nc(OC[C@@H]2CCCN2C)nc2c(F)c(-c3ccc(F)c4sc(N)nc34)c(Cl)cc12] | LCMS m/z [M+1]: 560.2 1HNMR(400 MHz, CD3OD): δ 7.93 (s, 1H) 7.22-7.24 (m, 1H) 7.05(t, J = 7.6 Hz, 1H) 4.68-4.71(m, 1H), 4.31-4.36 (m, 1H), 3.89 (t, J = 11.6 Hz, 2H) 3.71-3.75 (m, 1H) 3.49-3.5 (m, 2H), 3.35-3.45 (m, 5H), 3.11 (s, 3H), 2.40-2.41 (m, 1H), 2.01-2.20 (m, 3H), 1.59 (d, J = 7.6 Hz, 3H). |
| 78a | 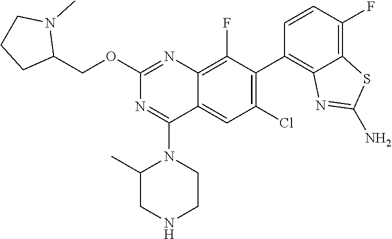 [SMILES: CC1CNCCN1c1nc(OCC2CCCN2C)nc2c(F)c(-c3ccc(F)c4sc(N)nc34)c(Cl)cc12] | |
| 79 | 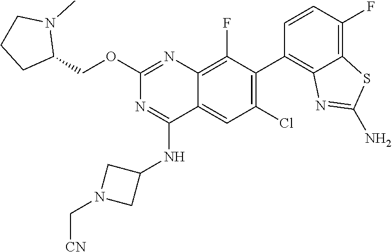 [SMILES: CN1CCC[C@H]1COc1nc(NC2CN(CC#N)C2)c2cc(Cl)c(-c3ccc(F)c4sc(N)nc34)c(F)c2n1] | LCMS m/z [M + 1]: 571.2 ¹HNMR (400 MHz, CD₃OD) δ 8.19 (s, 1H), 7.18-7.29 (m, 1H), 7.02 (t, J = 8.8 Hz, 1H), 5.00-5.31 (m, 2H), 4.69- 4.79 (m, 2H), 4.42-4.51 (m, 1H), 3.90- 3.97 (m, 1H), 3.67-3.87 (m, 3H), 3.16- 3.26 (m, 2H), 3.07-3.11 (m, 4H), 2.42- 2.47 (m, 1H), 2.04-2.29 (m, 3H). |
| 79a | 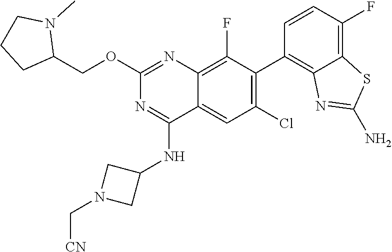 [SMILES: CN1CCCC1COc1nc(NC2CN(CC#N)C2)c2cc(Cl)c(-c3ccc(F)c4sc(N)nc34)c(F)c2n1] | |
| 80 | 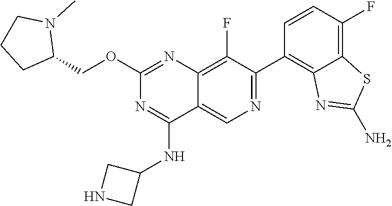 [SMILES: CN1CCC[C@H]1COc1nc(NC2CNC2)c2cnc(-c3ccc(F)c4sc(N)nc34)c(F)c2n1] | LCMS m/z [M + 1]: 499.2 ¹HNMR (300 MHz, CD₃OD) δ 9.37 (s, 1H), 7.77-7.42 (m, 1H), 7.12 (t, J = 8.8 Hz, 1H), 5.26 (m, 1H), 4.95 (m, 1H), 4.73 (m, 1H), 4.60-4.27 (m, 4H), 3.92 (m, 1H), 3.76 (m, 1H), 3.30-3.21 (m, 1H), 3.14 (s, 3H), 2.43 (m, 1H), 2.14 (m, 3H). |
| 80a | 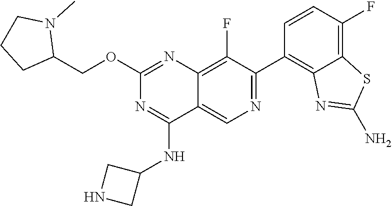 [SMILES: CN1CCCC1COc1nc(NC2CNC2)c2cnc(-c3ccc(F)c4sc(N)nc34)c(F)c2n1] | |
| 81 | 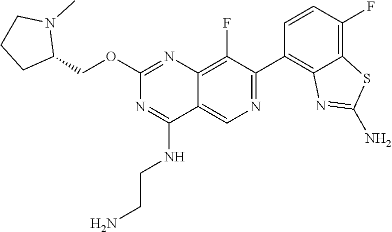 [SMILES: CN1CCC[C@H]1COc1nc(NCCN)c2cnc(-c3ccc(F)c4sc(N)nc34)c(F)c2n1] | LCMS m/z [M + 1]: 487.1 ¹HNMR (300 MHz, CD₃OD) δ 9.26 (s, 1H), 7.65 (dd, J = 8.5, 5.2 Hz, 1H), 7.13 (t, J = 8.7 Hz, 1H), 4.99 (m, 1H), 4.74 (dd, J = 13.2, 6.5 Hz, 1H), 4.03 (t, J = 5.9 Hz, 2H), 3.92 (m, 1H), 3.79 (m, 1H), 3.37 (t, J = 6.1 Hz, 2H), 3.27 (m, 1H), 3.14 (s, 3H), 2.45 (m, 1H), 2.12 (m, 3H). |
| 81a | 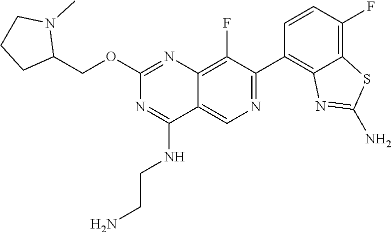 [SMILES: CN1CCCC1COc1nc(NCCN)c2cnc(-c3ccc(F)c4sc(N)nc34)c(F)c2n1] | |
| 82 | 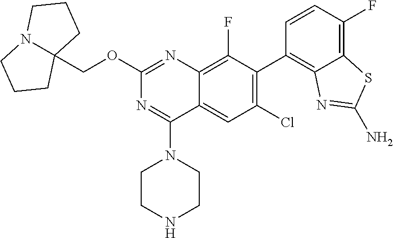 [SMILES: Nc1nc2c(-c3c(Cl)cc4c(N5CCNCC5)nc(OCC56CCCN5CCC6)nc4c3F)ccc(F)c2s1] | LCMS m/z [M + 1]: 572.3 ¹HNMR (400 MHz, CD₃OD): δ 8.00 (s, 1H), 7.21-7.24 (m, 1H), 7.02 (t, J = 8.8 Hz, 1H), 4.67 (s, 2H), 4.12 (t, J = 4.4 Hz, 4H), 3.65-3.71 (m, 2H), 3.49 (t, J = 4.4 Hz, 4H), 3.30-3.31 (m, 2H), 2.29-2.33 (m, 2H), 2.20-2.27 (m, 2H), 2.08-2.16 (m, 4H). |
| 83 | 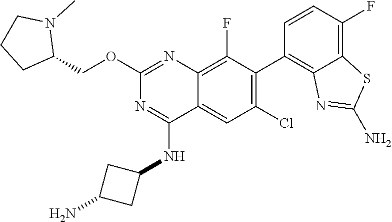 | LCMS m/z [M + 1]: 546.2 ¹HNMR (400 MHz, CD₃OD): δ 8.30 (s, 1H), 7.24 (dd, J = 5.6, 2.8 Hz, 1H), 7.03 (dd, J = 8.4, 2.0 Hz, 1H), 4.92-5.01 (m, 2H), 4.62-4.68 (m, 1H), 3.99-4.02 (m, 1H), 3.88 (s, 1H), 3.71 (s, 1H) , 3.07(s, 3H), 2.69-2.74 (m, 4H), 2.36-2.40 (m, 1H), 2.17-2.20 (m, 2H), 2.0-2.09 (m, 2H). |
| 83a | 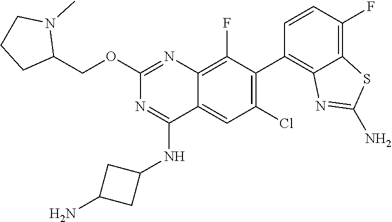 [SMILES: CN1CCCC1COc1nc(NC2CC(N)C2)c2cc(Cl)c(-c3ccc(F)c4sc(N)nc34)c(F)c2n1] | |
| 84 | 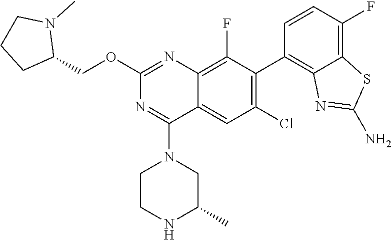 [SMILES: C[C@H]1CN(c2nc(OC[C@@H]3CCCN3C)nc3c(F)c(-c4ccc(F)c5sc(N)nc45)c(Cl)cc23)CCN1] | LCMS m/z [M + 1]: 560.3 ¹HNMR (400 MHz, CD₃OD) δ 7.99 (s, 1H), 7.21 (t, J = 6.8 Hz, 1H), 7.01 (t, J = 8.8 Hz, 1H), 4.61-4.70 (m,1H), 4.41- 4.56 (m, 2H), 3.81-3.90 (m, 1H), 3.70- 3.79 (m, 1H), 3.62-3.60 (m, 2H), 3.59 (s, 3H), 3.40-3.51 (m, 2H), 3.11 (s, 3H), 2.32-2.50 (m, 1H), 2.02-2.30 (m, 3H), 1.43 (d, J = 7.6 Hz, 3H). |
| 84a | 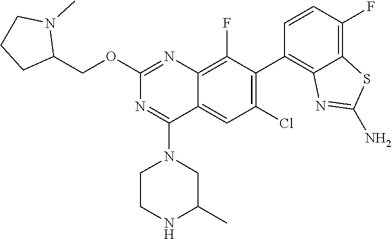 [SMILES: CC1CN(c2nc(OCC3CCCN3C)nc3c(F)c(-c4ccc(F)c5sc(N)nc45)c(Cl)cc23)CCN1] | |
| 85 | 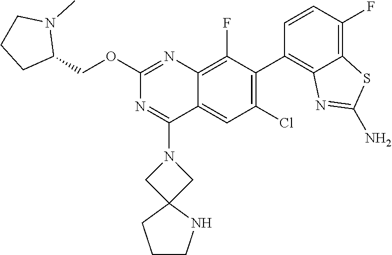 [SMILES: CN1CCC[C@H]1COc1nc(N2CC3(CCCN3)C2)c2cc(Cl)c(-c3ccc(F)c4sc(N)nc34)c(F)c2n1] | LCMS m/z [M + 1]: 572.2 ¹HNMR (400 MHz, CD₃OD) δ 7.83 (s, 1H), 7.22 (t, J = 6.0 Hz, 1H), 7.02 (t, J = 8.8 Hz, 1H), 4.61-4.70 (m, 1H), 3.88 (s, 1H), 3.71 (s, 1H), 3.49 (t, J = 7.4 Hz, 2H), 3.07 (s, 3H), 2.38-2.50 (m, 3H), 2.02-2.30(m, 5H). |
| 85a | 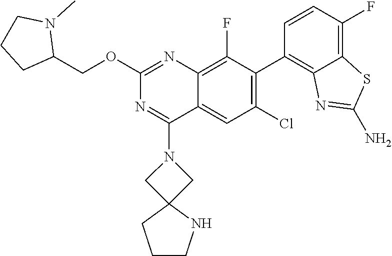 [SMILES: CN1CCCC1COc1nc(N2CC3(CCCN3)C2)c2cc(Cl)c(-c3ccc(F)c4sc(N)nc34)c(F)c2n1] | |
| 86 | 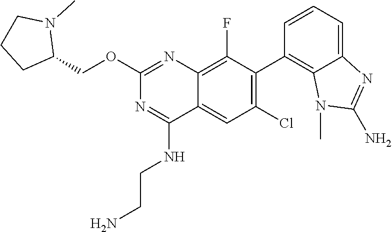 [SMILES: CN1CCC[C@H]1COc1nc(NCCN)c2cc(Cl)c(-c3cccc4nc(N)n(C)c34)c(F)c2n1] | LCMS m/z [M + 1]: 499.3 ¹HNMR (400 MHz, CD³OD) δ 8.23 (s, 1H), 7.55 (d, J = 8.0 Hz, 1H), 7.46 (t, J = 7.9 Hz, 1H), 7.18 (d, J = 8.0 Hz, 1H), 4.86-4.92 (m, 2H), 4.52-4.76 (m, 2H), 3.9-4.10 (m, 3H), 3.65-3.79 (m, 1H), 3.32-3.41 (m, 2H), 3.22 (s, 3H), 3.10 (s, 3H), 2.32-2.41 (m, 1H), 2.01-2.27 (m, 2H). |
| 86a | 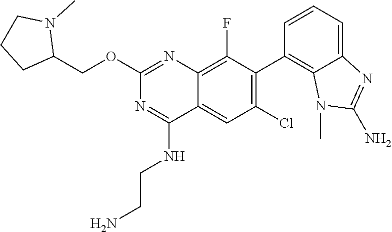 [SMILES: CN1CCCC1COc1nc(NCCN)c2cc(Cl)c(-c3cccc4nc(N)n(C)c34)c(F)c2n1] | |
| 87 | 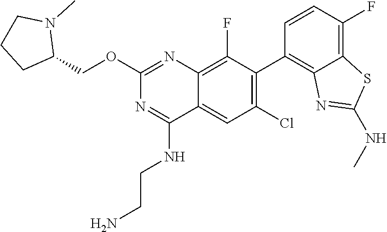 [SMILES: CNc1nc2c(-c3c(Cl)cc4c(NCCN)nc(OC[C@@H]5CCCN5C)nc4c3F)ccc(F)c2s1] | LCMS m/z [M + 1]: 534.2 |
| 87a | 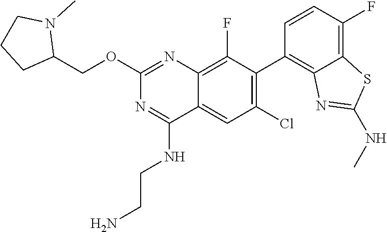 [SMILES: CNc1nc2c(-c3c(Cl)cc4c(NCCN)nc(OCC5CCCN5C)nc4c3F)ccc(F)c2s1] | |
| 89 | 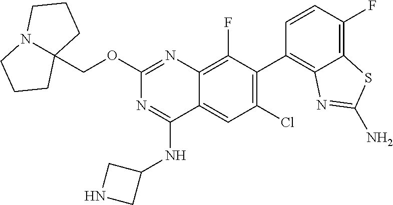 [SMILES: Nc1nc2c(-c3c(Cl)cc4c(NC5CNC5)nc(OCC56CCCN5CCC6)nc4c3F)ccc(F)c2s1] | LCMS m/z [M + 1]: 558.3 ¹HNMR (400 MHz, CD₃OD) δ 8.24 (s, 1H), 7.24 (dd, J = 8.4, 5.4 Hz, 1H), 7.03 (t, J = 8.8 Hz, 1H), 5.13-5.28 (m, 1H), 4.58-4.70 (m, 2H), 4.42-4.53 (m, 2H), 4.33-4.38 (m, 2H), 3.64-3.74 (m, 2H), 3.25-3.30 (m, 2H), 2.26-2.38 (m, 2H), 2.17-2.27 (m, 2H), 2.05-2.17 (m, 4H). |
| 91 | 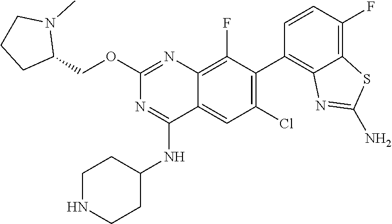 [SMILES: CN1CCC[C@H]1COc1nc(NC2CCNCC2)c2cc(Cl)c(-c3ccc(F)c4sc(N)nc34)c(F)c2n1] | LCMS m/z [M + 1]: 560.2 ¹HNMR (400 MHz, CD₃OD) δ 8.32 (s, 1H), 7.23-7.27 (m, 1H), 7.01-7.06 (dd, J = 8.8, 2.4 Hz, 1H), 4.85-4.92 (m, 1H), 4.65-4.69 (m, 1H), 4.50-4.58(m, 1H), 3.70-3.91 (m, 2H), 3.54 (d, J = 12.8 Hz, 2H), 3.20-3.30 (m, 3H), 3.08 (s, 3H), 2.33-2.41 (m, 3H), 1.97-2.19 (m, 5H). |
| 91a | 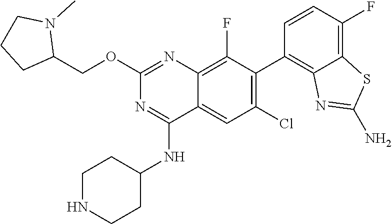 [SMILES: CN1CCCC1COc1nc(NC2CCNCC2)c2cc(Cl)c(-c3ccc(F)c4sc(N)nc34)c(F)c2n1] | |
| 92 | 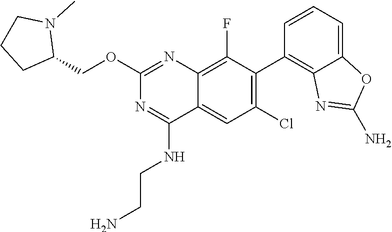 [SMILES: CN1CCC[C@H]1COc1nc(NCCN)c2cc(Cl)c(-c3cccc4oc(N)nc34)c(F)c2n1] | LCMS m/z [M+1]: 486.2 1HNMR(400 MHz, CD₃OD): δ 8.10 (s, 1H), 7.38 (d, J = 6.8 Hz, 1H), 7.11-7.15 (m, 2H), 4.42-4.85 (m, 2H), 3.74 (t, J = 6.4 Hz, 1H), 3.09-3.13 (m, 2H), 2.99- 3.08 (m, 2H), 2.71-2.90 (m, 1H), 2.52 (s, 3H), 2.35-2.40 (m, 1H), 2.08-2.23 (m, 1H), 1.62-1.90 (m, 3H). |
| 92a | 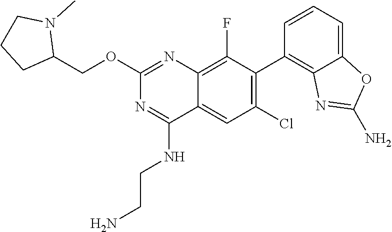 [SMILES: CN1CCCC1COc1nc(NCCN)c2cc(Cl)c(-c3cccc4oc(N)nc34)c(F)c2n1] | |
| 93 | 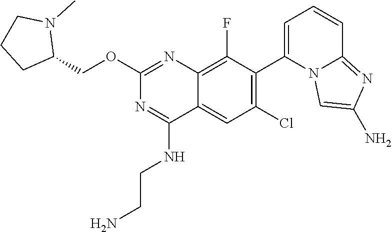 [SMILES: CN1CCC[C@H]1COc1nc(NCCN)c2cc(Cl)c(-c3cccc4nc(N)cn34)c(F)c2n1] | LCMS m/z [M + 1]: 485.1 ¹HNMR (400 MHz, CD₃OD) δ 8.30 (s, 1H), 7.78-7.87 (m, 3H), 7.48 (d, J = 6.8 Hz, 1H), 4.85-4.93 (m, 1H), 4.63- 4.69 (m, 1H), 3.90-4.01 (m, 2H), 3.75- 3.88 (m, 2H), 3.30-3.36 (m, 2H), 3.25- 3.29 (m, 1H), 3.10 (s, 3H), 2.37-2.43 (m, 1H), 2.09-2.20 (m, 3H). |
| 93a | 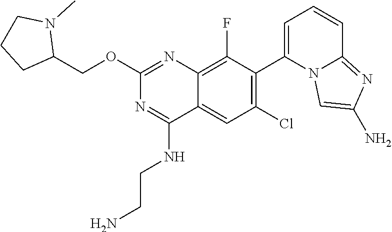 [SMILES: CN1CCCC1COc1nc(NCCN)c2cc(Cl)c(-c3cccc4nc(N)cn34)c(F)c2n1] | |
| 95 | 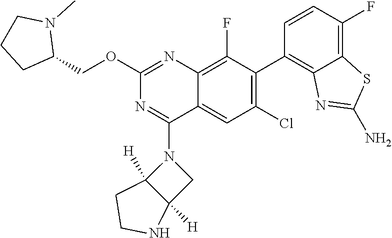 [SMILES: CN1CCC[C@H]1COc1nc(N2C[C@H]3NCC[C@H]32)c2cc(Cl)c(-c3ccc(F)c4sc(N)nc34)c(F)c2n1] | |
| 95a | 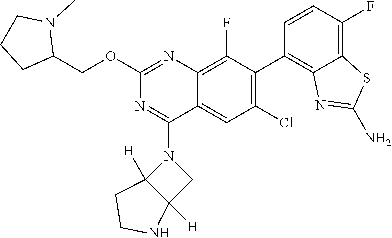 [SMILES: CN1CCCC1COc1nc(N2CC3NCCC32)c2cc(Cl)c(-c3ccc(F)c4sc(N)nc34)c(F)c2n1] | |
| 96 | 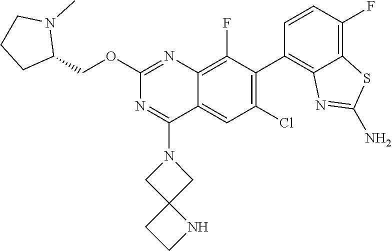 [SMILES: CN1CCC[C@H]1COc1nc(N2CC3(CCN3)C2)c2cc(Cl)c(-c3ccc(F)c4sc(N)nc34)c(F)c2n1] | |
| 96a | 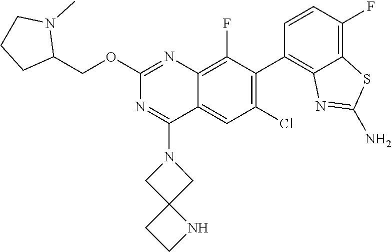 [SMILES: CN1CCCC1COc1nc(N2CC3(CCN3)C2)c2cc(Cl)c(-c3ccc(F)c4sc(N)nc34)c(F)c2n1] | |
| 97 | 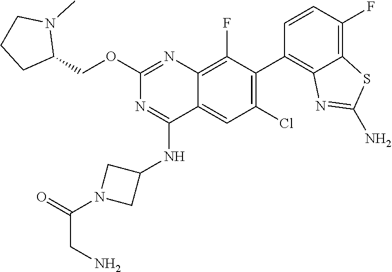 [SMILES: CN1CCC[C@H]1COc1nc(NC2CN(C(=O)CN)C2)c2cc(Cl)c(-c3ccc(F)c4sc(N)nc34)c(F)c2n1] | |
| 97a | 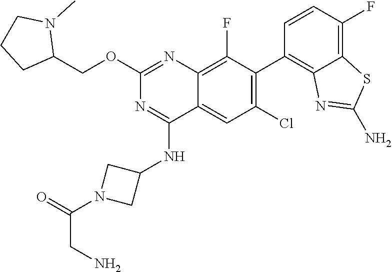 [SMILES: CN1CCCC1COc1nc(NC2CN(C(=O)CN)C2)c2cc(Cl)c(-c3ccc(F)c4sc(N)nc34)c(F)c2n1] | |
| 98 | 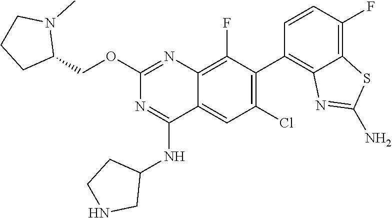 [SMILES: CN1CCC[C@H]1COc1nc(NC2CCNC2)c2cc(Cl)c(-c3ccc(F)c4sc(N)nc34)c(F)c2n1] | |
| 98a | 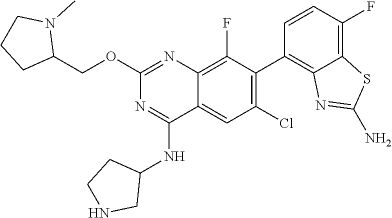 [SMILES: CN1CCCC1COc1nc(NC2CCNC2)c2cc(Cl)c(-c3ccc(F)c4sc(N)nc34)c(F)c2n1] | |
| 99 | 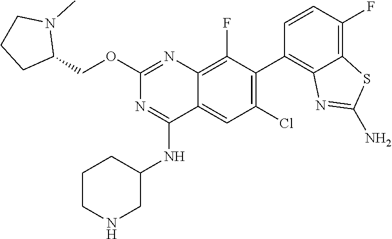 [SMILES: CN1CCC[C@H]1COc1nc(NC2CCCNC2)c2cc(Cl)c(-c3ccc(F)c4sc(N)nc34)c(F)c2n1] | |
| 99a | 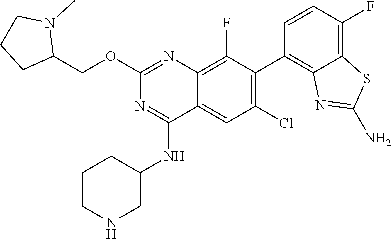 [SMILES: CN1CCCC1COc1nc(NC2CCCNC2)c2cc(Cl)c(-c3ccc(F)c4sc(N)nc34)c(F)c2n1] | |
| 100 | 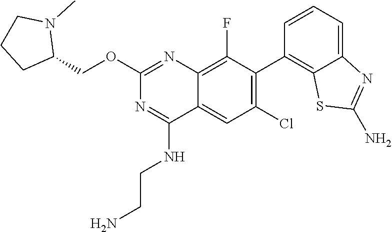 [SMILES: CN1CCC[C@H]1COc1nc(NCCN)c2cc(Cl)c(-c3cccc4nc(N)sc34)c(F)c2n1] | |
| 100a | 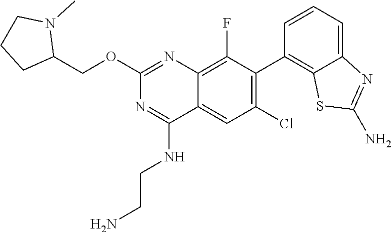 [SMILES: CN1CCCC1COc1nc(NCCN)c2cc(Cl)c(-c3cccc4nc(N)sc34)c(F)c2n1] | |
| 104 | 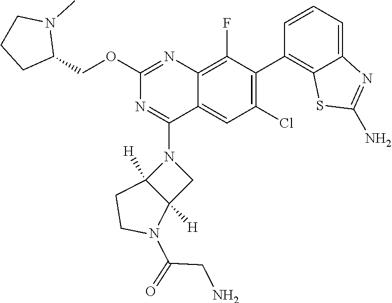 [SMILES: CN1CCC[C@H]1COc1nc(N2C[C@@H]3[C@H]2CCN3C(=O)CN)c2cc(Cl)c(-c3cccc4nc(N)sc34)c(F)c2n1] | |
| 104a | 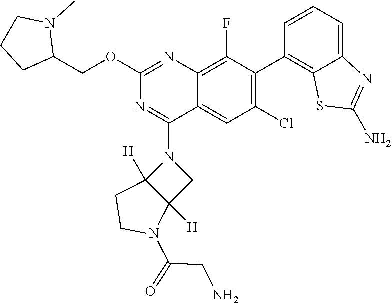 [SMILES: CN1CCCC1COc1nc(N2CC3C2CCN3C(=O)CN)c2cc(Cl)c(-c3cccc4nc(N)sc34)c(F)c2n1] | |
| 105 | 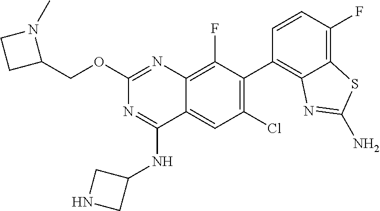 [SMILES: CN1CCC1COc1nc(NC2CNC2)c2cc(Cl)c(-c3ccc(F)c4sc(N)nc34)c(F)c2n1] | |
| 106 | 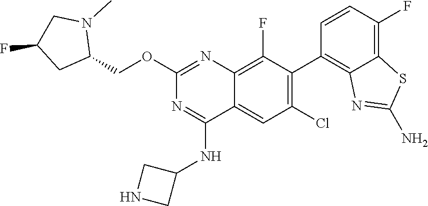 [SMILES: CN1C[C@H](F)C[C@H]1COc1nc(NC2CNC2)c2cc(Cl)c(-c3ccc(F)c4sc(N)nc34)c(F)c2n1] | |
| 106a | 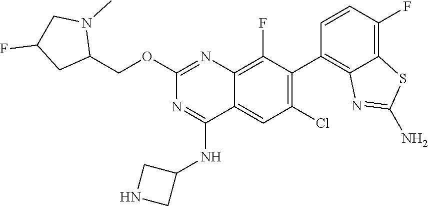 [SMILES: CN1CC(F)CC1COc1nc(NC2CNC2)c2cc(Cl)c(-c3ccc(F)c4sc(N)nc34)c(F)c2n1] | |
| 107 | 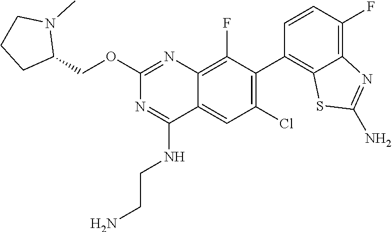 [SMILES: CN1CCC[C@H]1COc1nc(NCCN)c2cc(Cl)c(-c3ccc(F)c4nc(N)sc34)c(F)c2n1] | |
| 107a | 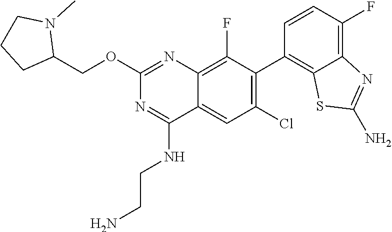 [SMILES: CN1CCCC1COc1nc(NCCN)c2cc(Cl)c(-c3ccc(F)c4nc(N)sc34)c(F)c2n1] | |
| 108 | 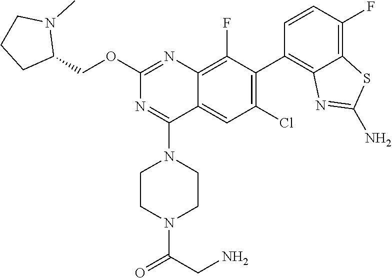 [SMILES: CN1CCC[C@H]1COc1nc(N2CCN(C(=O)CN)CC2)c2cc(Cl)c(-c3ccc(F)c4sc(N)nc34)c(F)c2n1] | |
| 108a | 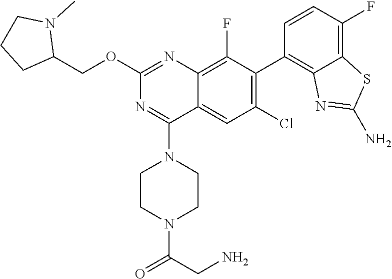 [SMILES: CN1CCCC1COc1nc(N2CCN(C(=O)CN)CC2)c2cc(Cl)c(-c3ccc(F)c4sc(N)nc34)c(F)c2n1] | |
| 109 | 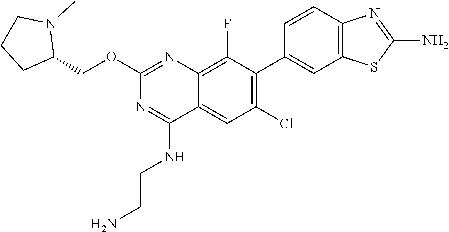 [SMILES: CN1CCC[C@H]1COc1nc(NCCN)c2cc(Cl)c(-c3ccc4nc(N)sc4c3)c(F)c2n1] | |
| 109a | 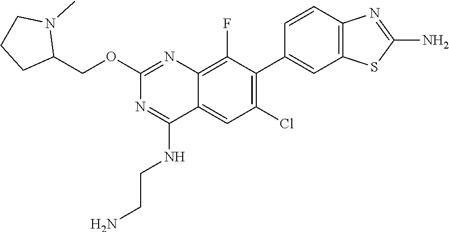 [SMILES: CN1CCCC1COc1nc(NCCN)c2cc(Cl)c(-c3ccc4nc(N)sc4c3)c(F)c2n1] | |
| 112 | 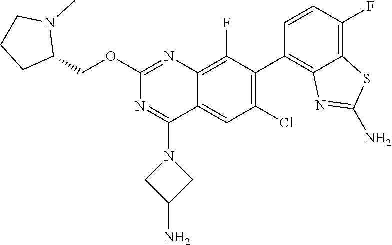 [SMILES: CN1CCC[C@H]1COc1nc(N2CC(N)C2)c2cc(Cl)c(-c3ccc(F)c4sc(N)nc34)c(F)c2n1] | |
| 112a | 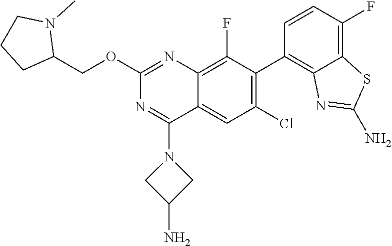 [SMILES: CN1CCCC1COc1nc(N2CC(N)C2)c2cc(Cl)c(-c3ccc(F)c4sc(N)nc34)c(F)c2n1] | |
| 113 | 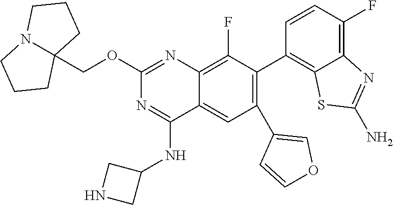 [SMILES: Nc1nc2c(F)ccc(-c3c(-c4ccoc4)cc4c(NC5CNC5)nc(OCC56CCCN5CCC6)nc4c3F)c2s1] | |
| 114 | 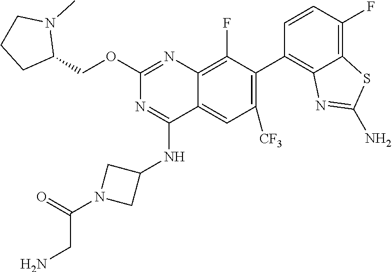 [SMILES: CN1CCC[C@H]1COc1nc(NC2CN(C(=O)CN)C2)c2cc(C(F)(F)F)c(-c3ccc(F)c4sc(N)nc34)c(F)c2n1] | |
| 114a | 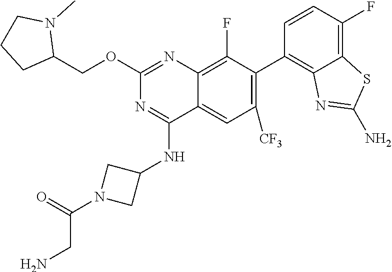 [SMILES: CN1CCCC1COc1nc(NC2CN(C(=O)CN)C2)c2cc(C(F)(F)F)c(-c3ccc(F)c4sc(N)nc34)c(F)c2n1] | |
| 115 | 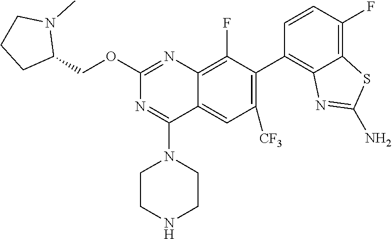 [SMILES: CN1CCC[C@H]1COc1nc(N2CCNCC2)c2cc(C(F)(F)F)c(-c3ccc(F)c4sc(N)nc34)c(F)c2n1] | |
| 115a | 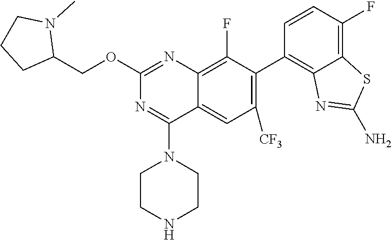 [SMILES: CN1CCCC1COc1nc(N2CCNCC2)c2cc(C(F)(F)F)c(-c3ccc(F)c4sc(N)nc34)c(F)c2n1] | |
| 116 | 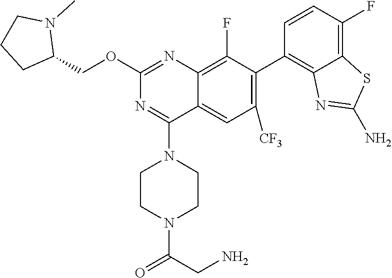 [SMILES: CN1CCC[C@H]1COc1nc(N2CCN(C(=O)CN)CC2)c2cc(C(F)(F)F)c(-c3ccc(F)c4sc(N)nc34)c(F)c2n1] | |
| 116a | 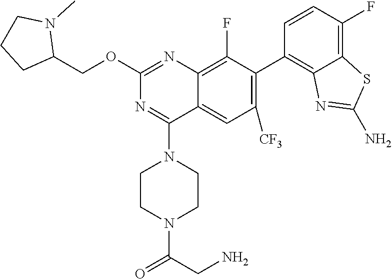 [SMILES: CN1CCCC1COc1nc(N2CCN(C(=O)CN)CC2)c2cc(C(F)(F)F)c(-c3ccc(F)c4sc(N)nc34)c(F)c2n1] | |

Additional compounds of the present disclosure are included in Table 4.

TABLE 4
Selected compounds of the present disclosure.

| Compound | | |
|---|---|---|
| No. | Structure | Analytical data |
| 46 | 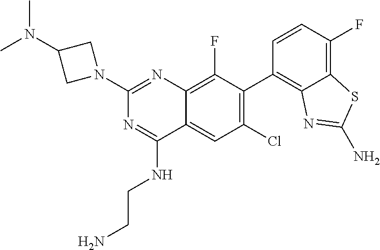 [SMILES: CN(C)C1CN(c2nc(NCCN)c3cc(Cl)c(-c4ccc(F)c5sc(N)nc45)c(F)c3n2)C1] | LCMS m/z [M + 1]: 505.1 ¹HNMR (400 MHz, CD₃OD): δ 8.16 (s, 1H), 7.21-7.25 (m, 1H), 7.02 (t, J = 8.8 Hz, 1H), 4.80-4.85 (m, 2H), 4.60-4.80 (m, 3H), 3.90- 4.10 (m, 2H), 3.58 (s, 6H), 3.35-3.42 (m, 2H). |
| 47 | 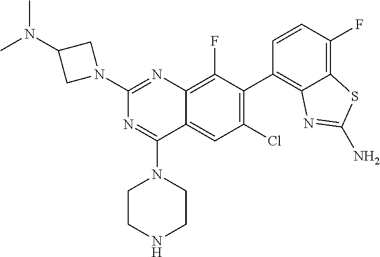 [SMILES: CN(C)C1CN(c2nc(N3CCNCC3)c3cc(Cl)c(-c4ccc(F)c5sc(N)nc45)c(F)c3n2)C1] | LCMS m/z [M + 1]: 531.20 ¹HNMR (400 MHz, CD₃OD): δ 7.84 (s,1H), 7.19 (t, J = 8.0 Hz, 1H), 7.00(t, J = 8.8 Hz, 1H), 4.55 (t, J = 8.0 Hz, 2H), 4.35-4.39 (m, 2H) 4.22-4.24 (m, 1H), 3.95-4.10(m, 4H) 3.40-3.52 (m, 4H), 2.95(s, 6H). |
| 101 | 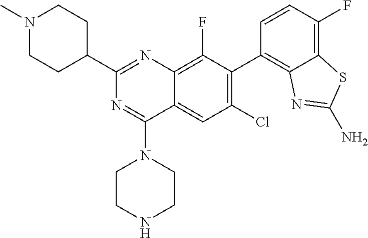 [SMILES: CN1CCC(c2nc(N3CCNCC3)c3cc(Cl)c(-c4ccc(F)c5sc(N)nc45)c(F)c3n2)CC1] | |
| 102 | 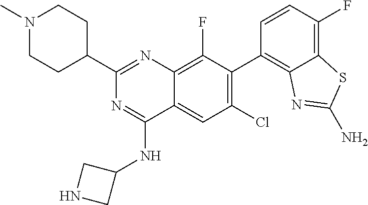 [SMILES: CN1CCC(c2nc(NC3CNC3)c3cc(Cl)c(-c4ccc(F)c5sc(N)nc45)c(F)c3n2)CC1] | |
| 103 | 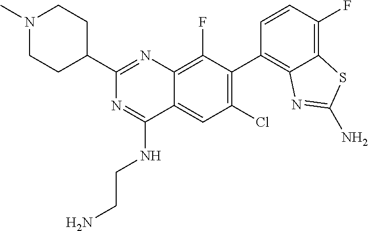 [SMILES: CN1CCC(c2nc(NCCN)c3cc(Cl)c(-c4ccc(F)c5sc(N)nc45)c(F)c3n2)CC1] | |

Additional compounds of the present disclosure are included in Table 5.

TABLE 5
Selected compounds of the present disclosure.

| Compound | | |
|---|---|---|
| No. | Structure | Analytical data |
| 34 | 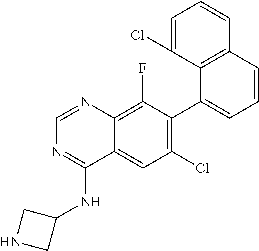 [SMILES: Fc1c(-c2cccc3cccc(Cl)c23)c(Cl)cc2c(NC3CNC3)ncnc12] | LCMS m/z [M + 1]: 413.1 ¹HNMR (400 MHz, CD₃OD): δ 8.71 (s, 1H), 8.31 (s, 1H), 8.15 (d, J = 8.3 Hz, 1H), 8.02 (d, J = 8.1 Hz, 1H), 7.69 (t, J = 7.8 Hz, 1H), 7.59 (d, J = 7.4 Hz, 1H), 7.40-7.56 (m, 2H), 5.06-5.23 (m, 1H), 4.39-4.62 (m, 4H). |
| 35 | 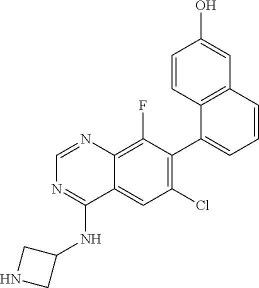 [SMILES: Oc1ccc2c(-c3c(Cl)cc4c(NC5CNC5)ncnc4c3F)cccc2c1] | LCMS m/z [M + 1]: 395.1 ¹HNMR (400 MHz, CD₃OD): δ 8.53 (s, 1H), 8.07 (s, 2H), 7.79 (d, J = 8.3 Hz, 1H), 7.50 (t, J = 7.6 Hz, 1H), 7.14-7.25 (m, 3H), 7.03 (ddd, J = 2.3, 1.9, 0.9 Hz, 1H), 4.59 (s, 2H), 4.41-4.48 (m, 1H), 4.02-4.10 (m, 1H), 3.02-3.10 (m, 1H). |
| 44 | 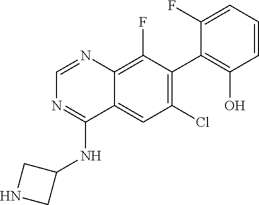 [SMILES: Oc1cccc(F)c1-c1c(Cl)cc2c(NC3CNC3)ncnc2c1F] | LCMS m/z[M + 1]: 363.1 ¹HNMR (400 MHz, CD₃OD): δ 8.69 (s, 1H), 8.36 (s, 1H), 7.35 (d, J = 7.20 Hz, 1H), 6.78 (d, J = 4.80 Hz, 1H), 6.74 (t, J = 8.80 Hz, 1H), 5.13-5.20 (m, 1H), 4.45-4.48 (m, 4H). |
| 45 | 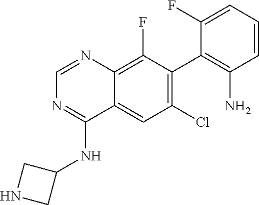 [SMILES: Nc1cccc(F)c1-c1c(Cl)cc2c(NC3CNC3)ncnc2c1F] | LCMS m/z [M + 1]: 362.1 ¹HNMR (400 MHz, CD₃OD): δ 8.68 (s, 1H), 8.35 (s, 1H), 7.20 (t, J = 7.20 Hz, 1H), 6.67 (d, J = 8.40 Hz, 1H), 6.48 (t, J = 8.40 Hz, 1H), 5.14 (t, J = 7.6 Hz, 1H), 4.44-4.49 (m, 4H). |
| 51 | 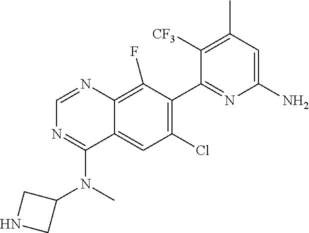 [SMILES: Cc1cc(N)nc(-c2c(Cl)cc3c(N(C)C4CNC4)ncnc3c2F)c1C(F)(F)F] | LCMS m/z [M + 1]: 441.1 ¹HNMR (400 MHz, CD₃OD): δ 8.86 (s, 1H), 8.61 (d, J = 1.4 Hz, 1H), 6.66 (s, 1H), 5.35 (d, J = 7.7 Hz, 1H), 4.51 (d, J = 11.5 Hz, 4H), 4.25 (d, J = 8.6 Hz, 3H), 2.46 (d, J = 1.0 Hz, 3H). |
| 52 | 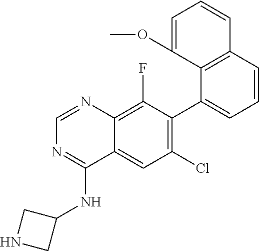 [SMILES: COc1cccc2cccc(-c3c(Cl)cc4c(NC5CNC5)ncnc4c3F)c12] | LCMS m/z [M + 1]: 409.1 ¹HNMR (400 MHz, CD₃OD): δ 8.70 (s, 1H), 8.28 (s, 1H), 8.00 (d, J = 8.3 Hz, 1H), 7.55- 7.63 (m, 2H), 7.47 (t, J = 7.9 Hz, 1H), 7.28 (d, J = 7.2 Hz, 1H), 6.92 (d, J = 7.9 Hz, 1H), 5.17 (t, J = 7.4 Hz, 1H), 4.43-4.56 (m, 4H), 3.46 (s, 3H). |
| 74 | 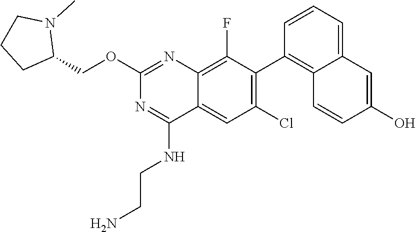 [SMILES: CN1CCC[C@H]1COc1nc(NCCN)c2cc(Cl)c(-c3cccc4cc(O)ccc34)c(F)c2n1] | LCMS m/z [M + 1]: 496.2 ¹HNMR(400 MHz, CD₃OD) δ 8.19 (s, 1H), 7.80 (d, J = 8.4 Hz, 1H), 7.50 (t, J = 7.6 Hz, 1H), 7.11-7.29 (m, 3H), 7.00 (dd, J = 8.8, 2.0 Hz, 1H) 4.61-4.69 (m, 2H), 3.81-3.97 (m, 3H), 3.51-3.70 (m, 2H), 3.07 (s, 3H), 2.32- 2.41 (m, 1H), 2.02-2.31(m, 3H), 1.21-1.30 (m, 2H). |

Additional compounds of the present disclosure are included in Table 6.

TABLE 6
Selected compounds of the present disclosure.

| Compound | | |
|---|---|---|
| No. | Structure | Analytical data |
| 39 | 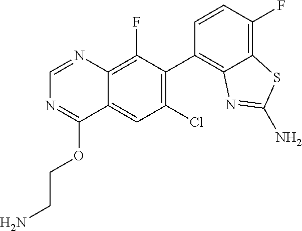 [SMILES: NCCOc1ncnc2c(F)c(-c3ccc(F)c4sc(N)nc34)c(Cl)cc12] | LCMS m/z [M + 1]: 408.1 ¹H NMR (400 MHz, DMSO-d6) δ: 8.53 (s, 1H), 8.42 (s, 1H), 7.92 (s, 1H), 7.24-7.28(m, 1H), 7.07 (t, J = 8.8 Hz, 1H), 3.49-3.65 (m, 4H). |
| 48 | 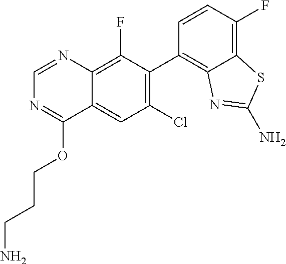 [SMILES: NCCCOc1ncnc2c(F)c(-c3ccc(F)c4sc(N)nc34)c(Cl)cc12] | LCMS m/z [M + 1]: 423.0 ¹HNMR (400 MHz, CD₃OD): δ 8.85 (s, 1H), 8.23 (s, 1H), 7.28 (dd, J = 8.4, 5.4 Hz, 1H), 7.05 (t, J = 8.8 Hz, 1H), 4.82 (t, J = 5.8 Hz, 2H), 3.18-3.35 (m, 2H), 2.17-2.42 (m, 2H). |
| 90 | 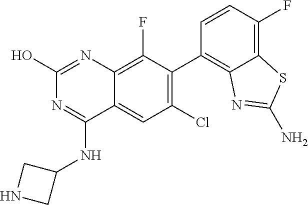 [SMILES: Nc1nc2c(-c3c(Cl)cc4c(NC5CNC5)nc(O)nc4c3F)ccc(F)c2s1] | LCMS m/z [M + 1]: 435.2 ¹HNMR (400 MHz, CD₃OD) δ 8.04 (s, 1H), 7.22 (dd, J = 8.4, 5.4 Hz, 1H), 7.00 (t, J = 8.8 Hz, 1H), 4.74-4.84 (m, 1H), 4.59-4.64 (m, 2H), 4.39-4.50 (m, 2H). |
| 90a | 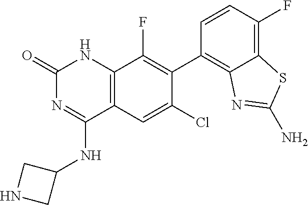 [SMILES: Nc1nc2c(-c3c(Cl)cc4c(NC5CNC5)nc(=O)[nH]c4c3F)ccc(F)c2s1] | |

Additional compounds of the present disclosure are included in Table 7.

TABLE 7
Selected compounds of the present disclosure.

| Compound | | |
|---|---|---|
| No. | Structure | Analytical data |
| 120 | 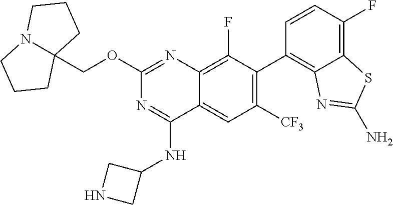 [SMILES: Nc1nc2c(-c3c(C(F)(F)F)cc4c(NC5CNC5)nc(OCC56CCCN5CCC6)nc4c3F)ccc(F)c2s1] | LCMS m/z [M + 1]: 592.3 ¹HNMR (400 MHz, CD₃OD): δ 8.57(s, 1H), 7.21-7.23 (m, 1H), 7.02 (t, J = 9.2 Hz, 1H), 5.23-5.27 (m, 1H), 4.66 (s, 2H), 4.46- 4.55(m, 2H), 4.32-4.43 (m, 2H), 3.62-3.78 (m, 2H), 3.23-3.34 (m, 2H), 2.08-2.32 (m, 8H). |
| 123 | 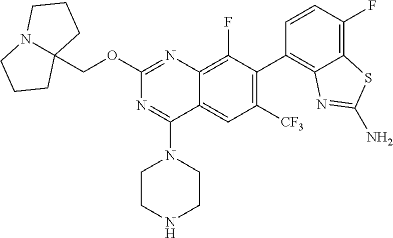 [SMILES: Nc1nc2c(-c3c(C(F)(F)F)cc4c(N5CCNCC5)nc(OCC56CCCN5CCC6)nc4c3F)ccc(F)c2s1] | LCMS m/z [M + 1]: 606.3 ¹HNMR(400 MHz, CD₃OD): δ 8.20(s, 1H), 7.19-7.22 (m, 1H), 7.00(t, J = 8.8 Hz, 1H), 4.69(s, 2H), 4.19-4.22(m, 4H), 3.63-3.74(m, 2H), 3.46-3.53(m, 4H), 3.21-3.29(m, 2H), 2.13-2.36(m, 8H). |
| 124 | 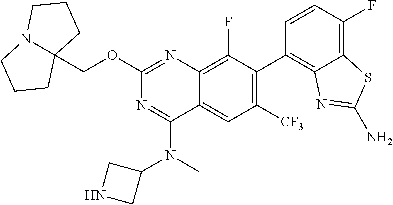 | LCMS m/z [M + 1]: 572.3 ¹HNMR (400 MHz, CD₃OD) δ 8.37 (d, J = 6.1 Hz, 1H), 7.22 (dd, J = 13.6, 8.2 Hz, 1H), 7.03 (t, J = 8.8 Hz, 1H), 4.96-5.07 (m, 2H), 4.77-4.87 (m, 2H), 3.88 (s, 3H), 3.58-3.77 (m, 5H), 3.30-3.35 (m, 2H), 2.36-2.40 (m, 2H), 2.21-2.26 (m, 2H), 2.17-2.18 (m, 4H). |
| 126 | 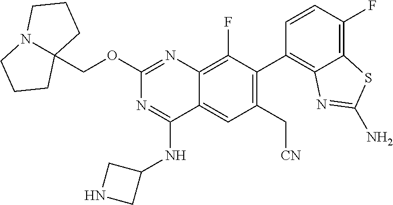 [SMILES: N#CCc1cc2c(NC3CNC3)nc(OCC34CCCN3CCC4)nc2c(F)c1-c1ccc(F)c2sc(N)nc12] | LCMS m/z [M + 1]: 563.4 ¹HNMR (400 MHz,CD₃OD) δ 8.18 (s, 1H), 7.28 (dd, J = 8.4, 5.4 Hz, 1H), 7.05 (t, J = 8.8 Hz, 1H), 5.21-5.31 (m, 1H), 4.63-4.73 (m, 2H), 4.47-4.56 (m, 2H), 4.36-4.45 (m, 2H), 3.75-3.88 (m, 2H), 3.64-3.74 (m, 2H), 3.24- 3.29 (m, 2H), 2.27-2.40 (m, 2H), 2.17-2.26 (m, 2H), 2.06-2.17 (m, 4H). |
| 127 | 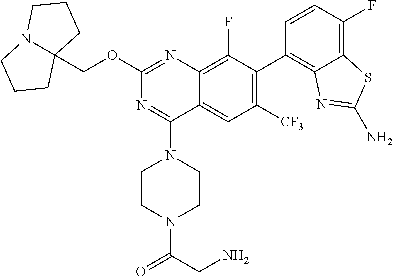 [SMILES: NCC(=O)N1CCN(c2nc(OCC34CCCN3CCC4)nc3c(F)c(-c4ccc(F)c5sc(N)nc45)c(C(F)(F)F)cc23)CC1] | LCMS m/z [M + 1]: 663.4 ¹HNMR (400 MHz, CD₃OD): δ 8.24 (s, 1H), 7.20-7.23 (m, 1H), 7.00 (t, J = 8.8 Hz, 1H), 4.68 (s, 2H), 4.11-4.15 (m, 4H), 4.03 (s, 2H), 3.85-3.95 (m, 2H), 3.72-3.80 (m, 2H), 3.62- 3.72 (m, 2H), 3.21-3.29 (m, 2H), 2.13-2.36 (m, 8H). |
| 153 | 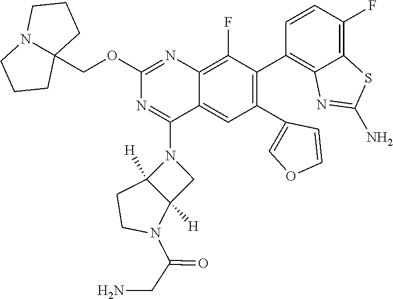 [SMILES: NCC(=O)N1CC[C@@H]2[C@H]1CN2c1nc(OCC23CCCN2CCC3)nc2c(F)c(-c3ccc(F)c4sc(N)nc34)c(-c3ccoc3)cc12] | LCMS m/z [M + 1]: 673.1 ¹HNMR (400 MHz, CD₃OD) δ 7.81 (s, 1H), 7.33 (s, 1H), 7.04-7.17 (m, 2H), 6.90-7.00 (m, 1H), 6.12 (d, J = 8.5 Hz, 1H), 5.54- 5.81 (m, 1H), 5.04 (s, 2H), 4.53-4.78 (m, 3H), 3.98-4.12 (m, 2H), 3.83-3.98 (m, 2H), 3.65-3.74 (m, 2H), 3.24-3.29 (m, 1H), 2.60-2.78 (m, 1H), 2.05-2.41 (m, 10H). |
| 157 | 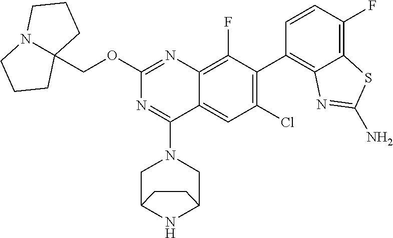 [SMILES: Nc1nc2c(-c3c(Cl)cc4c(N5CC6CCC(C5)N6)nc(OCC56CCCN5CCC6)nc4c3F)ccc(F)c2s1] | LCMS m/z [M + 1]: 598.2 ¹HNMR (400 MHz, CD₃OD) δ 7.95 (s, 1H), 7.22 (dd, J = 5.6, 8.4 Hz, 1H), 7.02 (t, J = 8.8 Hz, 1H), 4.68-4.75 (m, 2H), 4.65 (s, 2H), 4.18-4.32 (m, 2H), 3.84-3.92 (m, 2H), 3.63- 3.71 (m, 2H), 3.24-3.29 (m, 2H), 2.28-2.36 (m, 2H), 2.19-2.26 (m, 2H), 2.0-2.18 (m, 8H). |
| 158 | 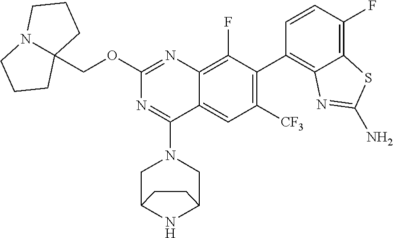 [SMILES: Nc1nc2c(-c3c(C(F)(F)F)cc4c(N5CC6CCC(C5)N6)nc(OCC56CCCN5CCC6)nc4c3F)ccc(F)c2s1] | LCMS m/z [M + 1]: 632.3 ¹H NMR (400 MHz, CD₃OD) δ 8.17 (s, 1H), 7.25-7.15 (m, 1H), 7.00 (t, J = 8.8 Hz, 1H), 4.77 (d, J = 13.6 Hz, 2H), 4.67 (s, 2H), 4.20- 4.30 (m, 2H), 3.95 (d, J = 14.0 Hz, 2H), 3.61-3.78 (m, 2H), 3.24-3.32 (m, 2H), 2.46- 1.97 (m, 12H). |
| 159 | 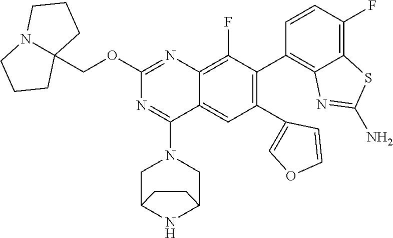 [SMILES: Nc1nc2c(-c3c(-c4ccoc4)cc4c(N5CC6CCC(C5)N6)nc(OCC56CCCN5CCC6)nc4c3F)ccc(F)c2s1] | LCMS m/z [M + 1]: 630.3 ¹HNMR (400 MHz, CD₃OD) δ 7.86 (s, 1H), 7.33 (s, 1H), 7.18 (s, 1H), 7.09 (dd, J = 8.4, 5.5 Hz, 1H), 6.95 (t, J = 8.8 Hz, 1H), 6.13 (s, 1H), 4.96-5.02 (m, 2H), 4.78 (d, J = 13.7 Hz, 2H), 4.68 (s, 2H), 4.19-4.21 (s, 2H), 3.90 (dd, J = 13.8, 8.6 Hz, 2H), 3.63-3.78 (m, 2H), 2.51-1.73 (m, 12H). |
| 161 | 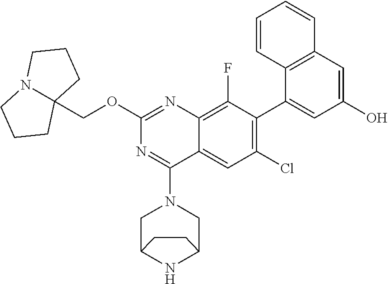 [SMILES: Oc1cc(-c2c(Cl)cc3c(N4CC5CCC(C4)N5)nc(OCC45CCCN4CCC5)nc3c2F)c2ccccc2c1] | LCMS m/z [M + 1]: 573.3 ¹HNMR (400 MHz, CD₃OD) δ 8.02 (s, 1H),, 7.76 (d, J = 6.36 Hz, 1H), 7.38-7.45(m, 1H), 7.28(s, 1H), 7.18-7.21(m, 2H), 7.01-7.03(m, 1H), 4.76(d, J = 13.6 Hz, 2H), 4.65-4.68(m, 2H), 4.21-4.29(m, 2H), 3.90(d, J = 14.4 Hz, 2H), 3.63-3.72(m, 2H), 3.25-3.28(m, 2H), 3.29-3.36(m, 2H), 2.19-2.26(m, 2H), 2.06- 2.18(m, 8H). |
| 162 | 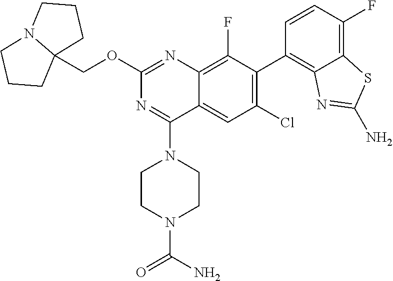 [SMILES: NC(=O)N1CCN(c2nc(OCC34CCCN3CCC4)nc3c(F)c(-c4ccc(F)c5sc(N)nc45)c(Cl)cc23)CC1] | LCMS m/z [M + 1]: 615.0 ¹HNMR (400 MHz, CD₃OD): δ 8.02 (s, 1H), 7.22-7.25 (m, 1H), 7.01 (t, J = 8.8 Hz, 1H), 4.65 (s, 2H), 3.96-4.11 (m, 4H), 3.61-3.75 (m, 6H), 3.24-3.28 (m, 2H), 2.06-2.36 (m, 8H). |
| 163 | 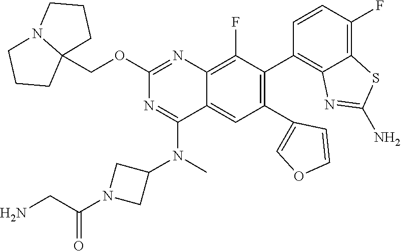 [SMILES: CN(c1nc(OCC23CCCN2CCC3)nc2c(F)c(-c3ccc(F)c4sc(N)nc34)c(-c3ccoc3)cc12)C1CN(C(=O)CN)C1] | LCMS m/z [M + 1]: 661.1 ¹HNMR (400 MHz, CD₃OD) δ 8.27 (s, 1H), 7.37 (s, 1H), 7.06 (s, 2H), 6.96 (t, J = 9.0 Hz, 1H), 6.24 (s, 1H), 5.09-5.35 (m, 4H), 4.74- 4.80 (m, 2H), 4.43-4.66 (m, 2H), 3.98-4.12 (m, 3H), 3.68-3.78 (m, 2H), 3.41-3.50 (m, 2H), 3.06-3.16 (m, 3H), 2.11-2.43 (m, 8H). |
| 164 | 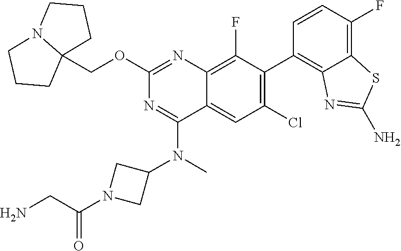 [SMILES: CN(c1nc(OCC23CCCN2CCC3)nc2c(F)c(-c3ccc(F)c4sc(N)nc34)c(Cl)cc12)C1CN(C(=O)CN)C1] | LCMS m/z [M + 1]: 629.1 ¹HNMR (400 MHz, CD3OD) δ 8.08 (s, 1H), 7.21-7.25 (m, 1H), 7.02 (t, J = 8.84 Hz, 1H), 5.00-5.06 (m, 1H), 4.59-4.67 (m, 3H), 4.43- 4.51 (m, 2H), 4.20-4.24 (m, 1H), 3.78-3.79 (m, 2H), 3.66-3.68 (m, 2H), 3.50 (s, 3H), 2.07-2.32 (m, 10H). |
| 165 | 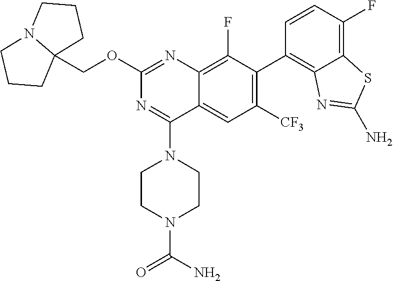 [SMILES: NC(=O)N1CCN(c2nc(OCC34CCCN3CCC4)nc3c(F)c(-c4ccc(F)c5sc(N)nc45)c(C(F)(F)F)cc23)CC1] | LCMS m/z [M + 1]: 649.2 ¹HNMR (400 MHz, CD₃OD): δ 8.26 (s, 1H), 7.22-7.25 (m, 1H), 7.02 (t, J = 8.8 Hz, 1H), 4.68 (s, 2H), 4.11-4.18 (m, 4H), 3.65-3.74 (m, 6H), 3.24-3.28 (m, 2H), 2.06-2.36 (m, 8H). |
| 167 | 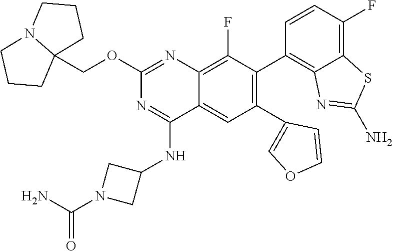 [SMILES: NC(=O)N1CC(Nc2nc(OCC34CCCN3CCC4)nc3c(F)c(-c4ccc(F)c5sc(N)nc45)c(-c4ccoc4)cc23)C1] | LCMS m/z [M + 1]: 633.1 ¹HNMR (400 MHz, CD₃OD) δ 8.27 (s, 1H), 7.34 (s, 1H), 7.09-7.12 (m, 1H), 7.03 (s, 1H), 6.96 (t, J = 8.9 Hz, 1H), 6.26 (s, 1H), 4.99-5.03 (m, 1H), 4.67-4.68 (m, 2H), 4.41 (t, J = 8.5 Hz, 2H), 4.18-4.22 (m, 2H), 3.68-3.70 (m, 2H), 2.02-2.36 (m, 8H). |
| 169 | 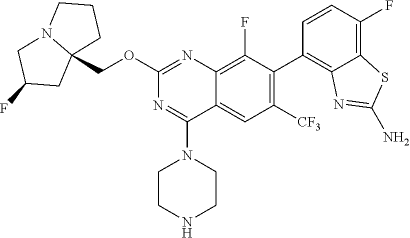 [SMILES: Nc1nc2c(-c3c(C(F)(F)F)cc4c(N5CCNCC5)nc(OC[C@]56CCCN5C[C@H](F)C6)nc4c3F)ccc(F)c2s1] | LCMS m/z [M + 1]: 624.1 ¹HNMR (400 MHz, CD₃OD) δ 8.21 (s, 1H), 7.17-7.27 (m, 1H), 7.01 (t, J = 8.8 Hz, 1H), 5.51 (d, J = 51.2 Hz, 1H), 4.69-4.88 (m, 2H), 4.15-4.30 (m, 4H), 3.98-4.10 (m, 1H) 3.61- 3.73 (m, 1H), 3.41-3.61 (m, 6H), 2.61-2.80 (m, 1H), 2.11-2.58 (m, 5H). |
| 171 | 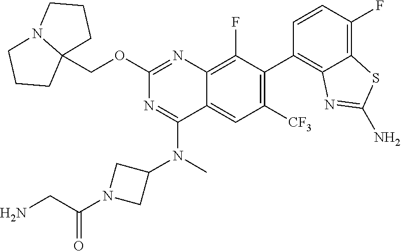 [SMILES: CN(c1nc(OCC23CCCN2CCC3)nc2c(F)c(-c3ccc(F)c4sc(N)nc34)c(C(F)(F)F)cc12)C1CN(C(=O)CN)C1] | LCMS m/z [M + 1]: 663.0 ¹H NMR (400 MHz, CD₃OD) δ 8.32 (s, 1H), 7.17-7.26 (m, 1H), 6.99 (t, J = 8.7 Hz, 1H), 5.03-5.17 (m, 2H), 4.58-4.72 (m, 3H), 4.47- 4.55 (m, 2H), 4.22-4.31 (m, 1H), 3.76-3.84 (m, 2H), 3.63-3.74 (m, 2H), 3.56 (s, 3H), 3.41-3.49 (m, 1H), 2.09-2.35 (m, 8H). |
| 173 | 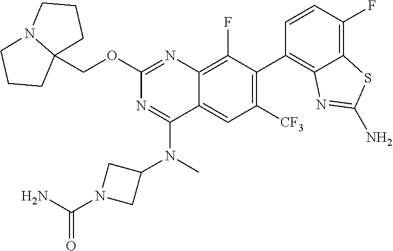 [SMILES: CN(c1nc(OCC23CCCN2CCC3)nc2c(F)c(-c3ccc(F)c4sc(N)nc34)c(C(F)(F)F)cc12)C1CN(C(N)=O)C1] | LCMS m/z [M + 1]: 649.1 ¹H NMR (400 MHz, CD₃OD) δ 8.64 (s, 1H), 7.16-7.24 (m, 1H), 7.00 (t, J = 8.8 Hz, 1H), 4.94-5.04 (m, 2H), 4.73-4.73 (m, 2H), 4.53-4.62 (m, 1H), 4.36 (dd, J = 23.1, 11.8 Hz, 1H), 4.00 (dd, J = 23.4, 10.6 Hz, 1H), 3.86-3.94 (m, 1H), 3.66-3.78 (m, 2H), 3.35- 3.35 (m, 1H), 3.01 (s, 3H), 2.25-2.47 (m, 4H), 2.12-2.21 (m, 4H). |
| 179 | 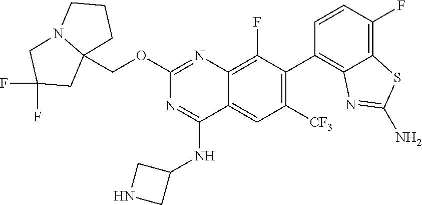 [SMILES: Nc1nc2c(-c3c(C(F)(F)F)cc4c(NC5CNC5)nc(OCC56CCCN5CC(F)(F)C6)nc4c3F)ccc(F)c2s1] | LCMS m/z [M + 1]: 594.1 ¹HNMR (500 MHz, CD₃OD) δ 8.27 (d, J = 1.4 Hz, 1H), 7.29 (ddd, J = 8.7, 5.4, 3.6 Hz, 1H), 7.08 (td, J = 8.8, 1.9 Hz, 1H), 5.22 (p, J = 7.7 Hz, 1H), 4.80-4.68 (m, 2H), 4.51 (dd, J = 11.2, 8.2 Hz, 2H), 4.37 (ddd, J = 11.5, 7.3, 4.3 Hz, 2H), 4.32-4.20 (m, 1H), 4.00- 3.78 (m, 3H), 3.46 (dtd, J = 10.0, 6.4, 3.5 Hz, 1H), 3.03-2.78 (m, 3H), 2.50-2.15 (m, 5H). |
| 181 | 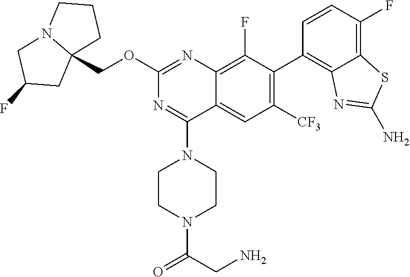 [SMILES: NCC(=O)N1CCN(c2nc(OC[C@]34CCCN3C[C@H](F)C4)nc3c(F)c(-c4ccc(F)c5sc(N)nc45)c(C(F)(F)F)cc23)CC1] | LCMS m/z [M + 1]: 681.0 ¹HNMR(400 MHz, CD₃OD): δ 8.24 (s, 1H), 7.20-7.28 (m, 1H), 6.99 (t, J = 8.8 Hz, 1H), 5.50 (d, J = 51.2 Hz, 1H), 4.70-4.82 (m, 2H), 4.01-4.14 (m, 7H), 3.80-3.90 (m, 2H), 3.63- 3.76 (m, 2H), 3.40-3.47 (m, 3H), 2.41-2.70 (m, 3H), 2.15-2.28 (m, 3H). |
| 182 | 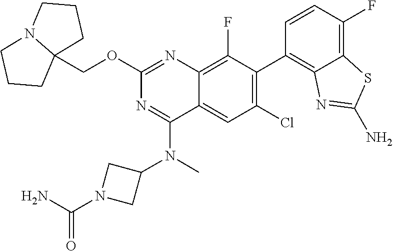 [SMILES: CN(c1nc(OCC23CCCN2CCC3)nc2c(F)c(-c3ccc(F)c4sc(N)nc34)c(Cl)cc12)C1CN(C(N)=O)C1] | LCMS m/z [M + 1]: 615.0 ¹HNMR (400 MHz, CD₃OD) δ 8.00 (s, 1H), 7.15-7.19 (m, 1H), 6.97 (t, J = 8.89 Hz, 1H), 4.63-4.73 (m, 2H), 4.48-4.53 (m, 1H), 4.11- 4.14 (m, 1H), 3.89-3.97 (m, 1H), 3.72-3.78 (m, 2H), 3.59-3.65 (m, 2H), 3.23-3.24 (m, 2H), 3.13 (s, 3H), 2.07-2.32 (m, 8H). |
| 184 | 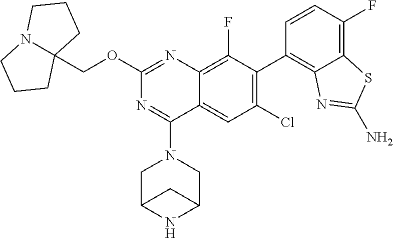 [SMILES: Nc1nc2c(-c3c(Cl)cc4c(N5CC6CC(C5)N6)nc(OCC56CCCN5CCC6)nc4c3F)ccc(F)c2s1] | LCMS m/z [M + 1]: 584.0 ¹HNMR (400 MHz, CD₃OD) δ 8.39 (d, J = 1.2 Hz, 1H), 7.26 (dd, J = 8.6, 5.6 Hz, 1H), 7.04 (t, J = 9.2 Hz, 1H), 4.83 (s, 1H), 4.79 (s, 1H), 4.68 (d, J = 2.8 Hz, 2H), 4.57-4.64 (m, 4H), 3.66-3.72 (m, 2H), 3.26-3.29 (m, 1H), 3.09-3.15 (m, 1H), 2.02-2.40 (m, 10H). |
| 185 | 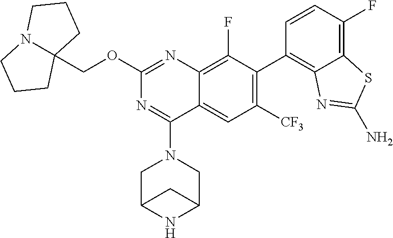 [SMILES: Nc1nc2c(-c3c(C(F)(F)F)cc4c(N5CC6CC(C5)N6)nc(OCC56CCCN5CCC6)nc4c3F)ccc(F)c2s1] | LCMS m/z [M + 1]: 618.1 ¹HNMR (400 MHz, CD₃OD) δ 8.60 (s, 1H), 7.20-7.23 (m, 1H), 7.00 (t, J = 8 Hz, 1H), 4.72-4.83 (m, 2H), 4.65 (s, 2H), 4.61-4.63 (m, 4H), 3.66-3.71 (m, 2H), 3.28-3.31 (m, 1H), 3.11-3.13 (m, 1H), 2.11-2.33 (m, 10H). |
| 186 | 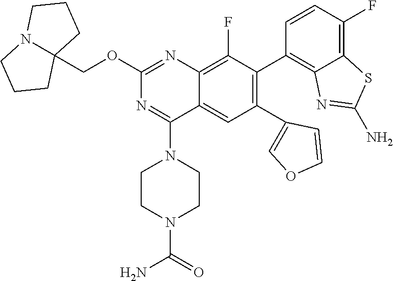 [SMILES: NC(=O)N1CCN(c2nc(OCC34CCCN3CCC4)nc3c(F)c(-c4ccc(F)c5sc(N)nc45)c(-c4ccoc4)cc23)CC1] | LCMS m/z [M + 1]: 647.0 ¹HNMR (400 MHz, CD₃OD) δ 7.97 (s, 1H), 7.33 (t, J = 1.6 Hz, 1H), 7.15 (s, 1H), 7.10 (dd, J = 8.4, 5.5 Hz, 1H), 6.95 (t, J = 8.8 Hz, 1H), 6.15 (d, J = 1.1 Hz, 1H), 4.60-4.77 (m, 2H), 4.00-4.28 (m, 4H), 3.64-3.81 (m, 6H), 2.06-2.37 (m, 8H). |
| 187 | 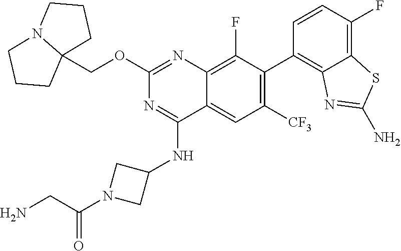 [SMILES: NCC(=O)N1CC(Nc2nc(OCC34CCCN3CCC4)nc3c(F)c(-c4ccc(F)c5sc(N)nc45)c(C(F)(F)F)cc23)C1] | LCMS m/z [M + 1]: 649.1 ¹HNMR (400 MHz, CD₃OD) δ 8.60 (s, 1H), 7.20 (dd, J = 8.3, 5.7 Hz, 1H), 6.99 (t, J = 8.8 Hz, 1H), 5.09-5.51 (m, 1H), 4.71 (t, J = 8.5 Hz, 1H), 4.65 (s, 2H), 4.55 (t, J = 8.9 Hz, 1H), 4.35 (dd, J = 9.3, 5.4 Hz, 1H), 4.24 (dd, J = 10.3, 5.1 Hz, 1H), 3.62-3.84 (m, 4H), 3.24-3.28 (m, 2H), 2.06-2.36 (m, 8H). |
| 188 | 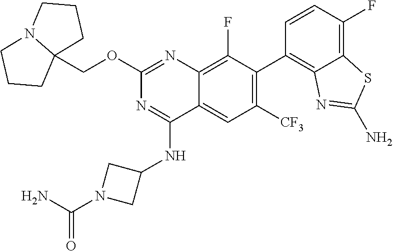 [SMILES: NC(=O)N1CC(Nc2nc(OCC34CCCN3CCC4)nc3c(F)c(-c4ccc(F)c5sc(N)nc45)c(C(F)(F)F)cc23)C1] | LCMS m/z [M + 1]: 635.2 ¹HNMR (400 MHz, CD₃OD) δ 8.62 (s, 1H), 7.22 (dd, J = 8.3, 5.5 Hz, 1H), 7.00 (t, J = 8.8 Hz, 1H), 4.95-5.02 (m, 2H), 4.65 (s, 2H), 4.40 (t, J = 8.2 Hz, 2H), 4.20 (dd, J = 8.7, 5.6 Hz, 2H), 3.65-3.74 (m, 2H), 3.24-3.29 (m, 1H), 2.28-2.36 (m, 2H), 2.05-2.27 (m, 6H). |
| 189 | 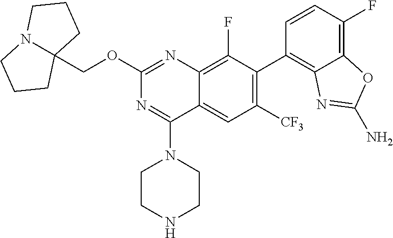 [SMILES: Nc1nc2c(-c3c(C(F)(F)F)cc4c(N5CCNCC5)nc(OCC56CCCN5CCC6)nc4c3F)ccc(F)c2o1] | LCMS m/z [M + 1]: 590.2 ¹HNMR (400 MHz, CD₃OD): δ 8.20 (s, 1H), 7.02-7.10 (m, 1H), 6.99 (t, J = 8.8 Hz, 1H), 4.69 (s, 2H), 4.17-4.22 (m, 4H), 3.65-3.74 (m, 2H), 3.46-3.51 (m, 4H), 3.24-3.32 (m, 2H), 2.08-2.39 (m, 8H). |
| 190 | 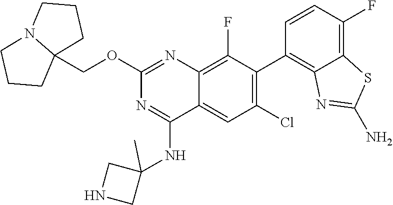 [SMILES: CC1(Nc2nc(OCC34CCCN3CCC4)nc3c(F)c(-c4ccc(F)c5sc(N)nc45)c(Cl)cc23)CNC1] | |
| 192 | 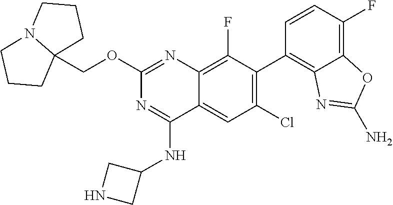 [SMILES: Nc1nc2c(-c3c(Cl)cc4c(NC5CNC5)nc(OCC56CCCN5CCC6)nc4c3F)ccc(F)c2o1] | LCMS m/z [M + 1]: 542.0 ¹HNMR (400 MHz, CD₃OD): δ 8.24 (s, 1H), 7.12 (dd, J = 4.8, 8.8 Hz, 1H), 7.01 (t, J = 10 Hz, 1H), 5.19 (t, J = 7.6 Hz, 1H), 4.60-4.64 (m, 2H), 4.43-4.54 (m, 2H), 4.31-4.41 (m, 2H), 3.66-3.75 (m, 2H), 3.20-3.28 (m, 2H), 2.11-2.33 (m, 8H). |
| 194 | 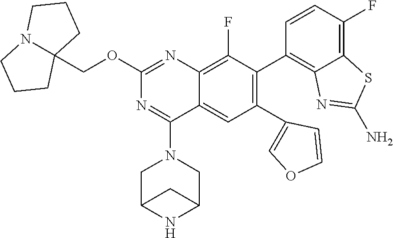 [SMILES: Nc1nc2c(-c3c(-c4ccoc4)cc4c(N5CC6CC(C5)N6)nc(OCC56CCCN5CCC6)nc4c3F)ccc(F)c2s1] | LCMS m/z [M + 1]: 616.2 ¹HNMR (400 MHz, CD₃OD) δ 8.32 (s, 1H), 7.33 (d, J = 1.6 Hz, 1H), 7.24 (s, 1H), 7.10- 7.15 (m, 1H), 6.95-7.01 (m, 1H), 6.14 (s, 1H), 4.89-4.90 (m, 1H), 4.68-4.77 (m, 3H), 4.65-4.67 (m, 1H), 4.62-4.65 (m, 2H), 3.66- 3.74 (m, 2H), 3.24-3.29 (m, 2H), 3.08-3.16 (m, 1H), 1.99-2.38 (m, 10H). |
| 195 | 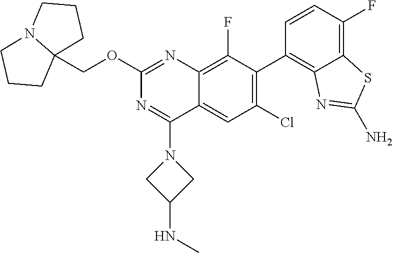 [SMILES: CNC1CN(c2nc(OCC34CCCN3CCC4)nc3c(F)c(-c4ccc(F)c5sc(N)nc45)c(Cl)cc23)C1] | LCMS m/z [M + 1]: 572.3 ¹HNMR (400 MHz, CD₃OD) δ 7.86 (s, 1H), 7.23-7.27 (m, 1H), 7.03-7.09 (m, 1H), 4.95- 5.10 (m, 2H), 4.72-4.84 (m, 2H), 4.61-4.72 (m, 2H), 4.30-4.39 (m, 1H), 3.63-3.74 (m, 2H), 3.23-3.28 (m,2H), 2.84 (s, 3H), 2.05- 2.36 (m, 8H). |
| 198 | 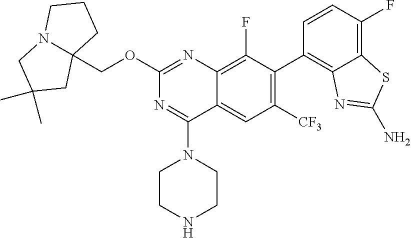 [SMILES: CC1(C)CN2CCCC2(COc2nc(N3CCNCC3)c3cc(C(F)(F)F)c(-c4ccc(F)c5sc(N)nc45)c(F)c3n2)C1] | LCMS m/z [M + 1]: 634.1 ¹HNMR (400 MHz, CD₃OD) δ 8.20 (s, 1H), 7.18-7.22 (m, 1H), 6.99 (t, J = 8.92 Hz, 1H), 4.69-4.78 (m, 2H), 4.18-4.20 (m, 4H), 3.58- 3.62 (m, 1H), 3.48-3.50 (m, 4H), 3.41-3.46 (m, 2H), 3.11-3.14 (m, 1H), 2.07-2.45 (m, 6H), 1.28 (s, 3H), 1.27 (s, 3H). |
| 199 | 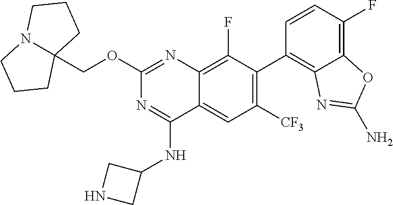 [SMILES: Nc1nc2c(-c3c(C(F)(F)F)cc4c(NC5CNC5)nc(OCC56CCCN5CCC6)nc4c3F)ccc(F)c2o1] | LCMS m/z [M + 1]: 576.2 ¹HNMR (400 MHz, CD₃OD) δ 8.56 (s, 1H), 7.07 (dd, J = 8.7, 4.6 Hz, 1H), 6.95-7.01 (m, 1H), 5.19-5.28 (m, 1H), 4.66 (s, 2H), 4.46- 4.54 (m, 2H), 4.33-4.41 (m, 2H), 3.63-3.72 (m, 2H), 3.25-3.28 (m, 1H), 2.27-2.36 (m, 2H), 2.18-2.26 (m, 2H), 2.07-2.17 (m, 4H). |
| 202 | 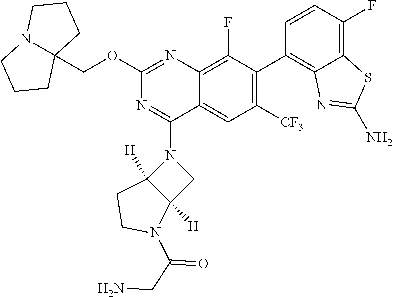 [SMILES: NCC(=O)N1CC[C@@H]2[C@H]1CN2c1nc(OCC23CCCN2CCC3)nc2c(F)c(-c3ccc(F)c4sc(N)nc34)c(C(F)(F)F)cc12] | LCMS m/z [M + 1]: 675.2 ¹HNMR (400 MHz, CD₃OD) δ 8.08 (s, 1H), 7.15-7.22 (m, 1H), 6.94-7.05 (m, 1H), 5.52- 5.72 (m, 1H), 4.42-4.68 (m, 3H), 3.78-4.10 (m, 1H), 3.35-3.75 (m, 5H), 2.83-3.10 (m, 2H), 2.52-2.71 (m, 1H), 1.85-2.36 (m, 9H). |
| 206 | 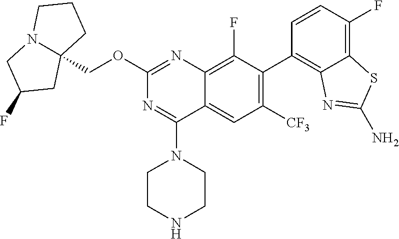 [SMILES: Nc1nc2c(-c3c(C(F)(F)F)cc4c(N5CCNCC5)nc(OC[C@@]56CCCN5C[C@H](F)C6)nc4c3F)ccc(F)c2s1] | LCMS m/z [M + 1]: 624.1 ¹HNMR (400 MHz, CD₃OD) δ 8.15 (s, 1H), 7.20-7.21 (m, 1H), 7.02 (t, J = 8 Hz, 1H), 5.51-5.63 (m, 1H), 5.01-5.11 (m, 1H), 4.67- 4.75 (m, 2H), 4.51 (d, J = 8 Hz, 1H), 3.88- 4.01 (m, 4H), 3.43-3.50 (m, 6H), 2.59-2.72 (m, 2H), 2.31-2.43 (m, 3H), 2.10-2.28 (m, 1H). |
| 206a | 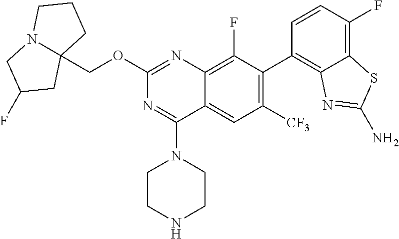 [SMILES: Nc1nc2c(-c3c(C(F)(F)F)cc4c(N5CCNCC5)nc(OCC56CCCN5CC(F)C6)nc4c3F)ccc(F)c2s1] | |
| 207 | 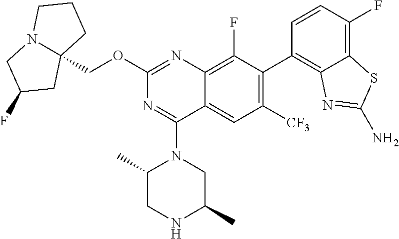 [SMILES: C[C@@H]1CN(c2nc(OC[C@@]34CCCN3C[C@H](F)C4)nc3c(F)c(-c4ccc(F)c5sc(N)nc45)c(C(F)(F)F)cc23)[C@@H](C)CN1] | LCMS m/z [M + 1]: 652.2 ¹HNMR (400 MHz, CD₃OD) δ 8.24 (s, 1H), 7.18-7.23 (m, 1H), 7.00 (t, J = 8.0 Hz, 1H), 5.51-5.64 (m, 1H), 4.63-4.72 (m, 3H), 3.86- 4.03 (m, 6H), 3.65-3.68 (m, 1H), 3.35-3.51 (m, 2H), 2.18-2.76 (m, 6H), 1.58(d, J = 8.0 Hz, 3H), 1.45-1.47 (m, 3H). |
| 207a | 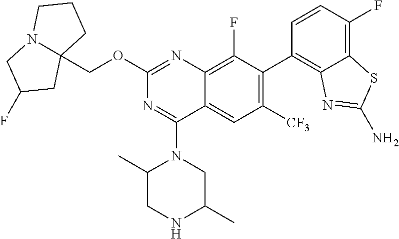 [SMILES: CC1CN(c2nc(OCC34CCCN3CC(F)C4)nc3c(F)c(-c4ccc(F)c5sc(N)nc45)c(C(F)(F)F)cc23)C(C)CN1] | |
| 209 | 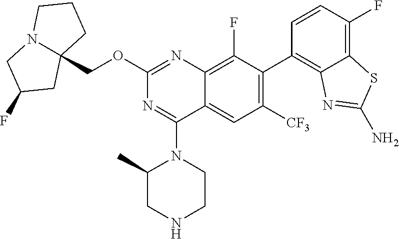 [SMILES: C[C@@H]1CNCCN1c1nc(OC[C@]23CCCN2C[C@H](F)C3)nc2c(F)c(-c3ccc(F)c4sc(N)nc34)c(C(F)(F)F)cc12] | LCMS m/z [M + 1]: 638.2 ¹HNMR (400 MHz, CD₃OD) δ 8.15 (s, 1H), 7.19-7.22 (m,1H), 6.99 (t, J = 8 Hz, 1H), 5.44-5.57 (m, 2H), 5.00-5.02 (m,2H), 4.76- 4.79 (m, 2H), 4.47-4.51 (m,1H), 3.90-4.08 (m, 2H), 3.65-3.67 (m, 1H), 3.43-3.53 (m, 6H), 2.68-2.75 (m, 1H), 2.37-2.53 (m,2H), 2.26-2.28 (m, 2H),2.13-2.16 (m, 1H), 1.65- 1.67 (m, 3H). |
| 209a | 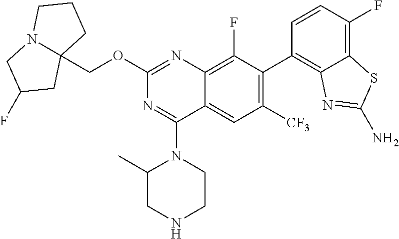 [SMILES: CC1CNCCN1c1nc(OCC23CCCN2CC(F)C3)nc2c(F)c(-c3ccc(F)c4sc(N)nc34)c(C(F)(F)F)cc12] | |
| 210 | 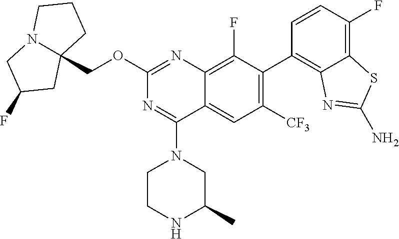 [SMILES: C[C@@H]1CN(c2nc(OC[C@]34CCCN3C[C@H](F)C4)nc3c(F)c(-c4ccc(F)c5sc(N)nc45)c(C(F)(F)F)cc23)CCN1] | LCMS m/z [M + 1]: 638.2 ¹HNMR (400 MHz, CD₃OD) δ 8.22 (s, 1H), 7.20-7.30 (m, 1H), 7.04 (t, J = 8.8 Hz, 1H), 5.47-5.60 (m, 1H), 4.75-4.85 (m, 3H), 4.55- 4.65 (m, 2H), 4.07 (t, J = 4.8 Hz, 1H), 3.82 (t, J = 4.8 Hz, 1H), 3.45-3.75 (m, 7H), 2.75 (t, J = 4.4 Hz, 1H), 2.15-2.60 (m, 5H), 1.32 (d, J = 6.4 Hz, 3H). |
| 210a | 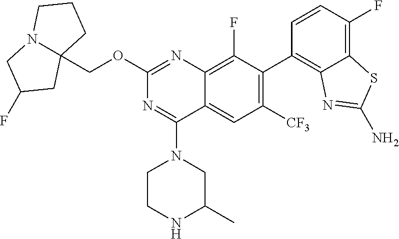 [SMILES: CC1CN(c2nc(OCC34CCCN3CC(F)C4)nc3c(F)c(-c4ccc(F)c5sc(N)nc45)c(C(F)(F)F)cc23)CCN1] | |
| 211 | 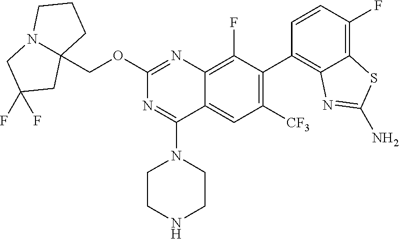 [SMILES: Nc1nc2c(-c3c(C(F)(F)F)cc4c(N5CCNCC5)nc(OCC56CCCN5CC(F)(F)C6)nc4c3F)ccc(F)c2s1] | LCMS m/z [M + 1]: 642.1 ¹HNMR (400 MHz, CD₃OD) δ 8.21 (s, 1H), 7.15-7.26 (m, 1H), 7.00 (t, J = 8.8 Hz, 1H), 4.76 (s, 2H), 4.09-4.40 (m, 4H), 3.68-4.06 (m, 2H), 3.41-3.58 (m, 4H), 3.32-3.41 (m, 2H), 2.71-3.09 (m, 2H), 2.14-2.52 (m, 4H). |
| 212 | 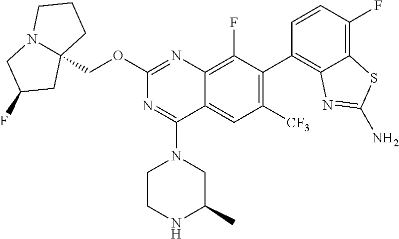 [SMILES: C[C@@H]1CN(c2nc(OC[C@@]34CCCN3C[C@H](F)C4)nc3c(F)c(-c4ccc(F)c5sc(N)nc45)c(C(F)(F)F)cc23)CCN1] | LCMS m/z [M + 1]: 638.2 ¹HNMR (400 MHz, CD₃OD) δ 8.20 (s, 1H), 7.17-7.24 (m, 1H), 7.03 (t, J = 8.8 Hz, 1H), 5.57 (d, J = 51.6 Hz, 1H), 4.69-4.78 (m, 2H), 4.56-4.61 (m, 1H), 3.82-4.08 (m, 2H) 3.40- 3.82 (m, 8H), 2.11-2.73 (m, 6H), 1.41-1.49 (d, J = 6.4 Hz, 3H). |
| 213 | 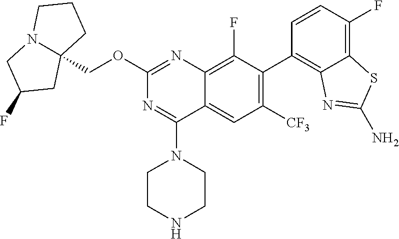 [SMILES: Nc1nc2c(-c3c(C(F)(F)F)cc4c(N5CCNCC5)nc(OC[C@@]56CCCN5C[C@H](F)C6)nc4c3F)ccc(F)c2s1] | LCMS m/z [M + 1]: 624.1 ¹H NMR (400 MHz, CD3OD) δ 8.20 (s, 1H), 7.17-7.24(m, 1H), 7.01(t, J = 8.8 Hz, 1H), 5.57(d, J = 55.2 Hz, 1H), 4.69-4.88(m, 2H), 4.15-4.30(m, 4H), 3.89-4.05(m, 3H) 3.45- 3.53(m, 5H), 2.11-2.73(m, 6H) |
| 214 | 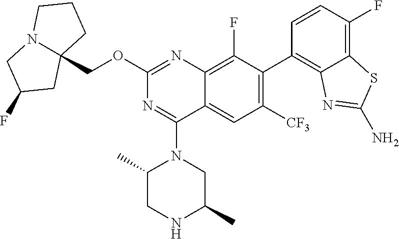 [SMILES: C[C@@H]1CN(c2nc(OC[C@]34CCCN3C[C@H](F)C4)nc3c(F)c(-c4ccc(F)c5sc(N)nc45)c(C(F)(F)F)cc23)[C@@H](C)CN1] | LCMS m/z [M + 1]: 652.2 ¹HNMR (400 MHz, CD₃OD) δ 8.24 (s, 1H), 7.21-7.26 (m, 1H), 6.99-7.04 (m, 1H), 5.44- 5.57 (m, 1H), 4.77-4.79 (m, 2H), 4.62-4.66 (m, 1H), 3.96-4.08 (m, 3H), 3.85-3.86 (m, 1H), 3.62-3.70 (m, 2H), 3.35-3.57 (m, 3H), 2.68-2.76 (m, 1H), 2.38-2.54 (m, 2H), 2.28- 2.30 (m, 2H), 2.14-2.18 (m, 1H), 1.58 (d, J = 6.3 Hz, 3H), 1.44 (d, J = 6.7 Hz, 3H). |
| 215 | 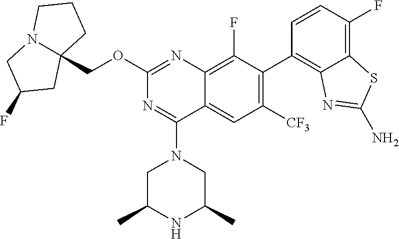 [SMILES: C[C@@H]1CN(c2nc(OC[C@]34CCCN3C[C@H](F)C4)nc3c(F)c(-c4ccc(F)c5sc(N)nc45)c(C(F)(F)F)cc23)C[C@H](C)N1] | LCMS m/z [M + 1]: 652.2 ¹HNMR (400 MHz, CD₃OD) δ 8.18 (s, 1H), 7.18-7.23 (m, 1H), 6.99 (t, J = 8.0 Hz, 1H), 5.45-5.57 (m, 1H), 4.63-4.75 (m, 3H), 3.87- 4.10 (m, 1H), 3.31-3.66 (m, 6H), 2.14-2.73 (m, 6H), 1.28-1.44 (m, 8H). |
| 215a | 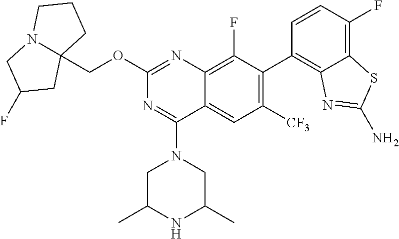 [SMILES: CC1CN(c2nc(OCC34CCCN3CC(F)C4)nc3c(F)c(-c4ccc(F)c5sc(N)nc45)c(C(F)(F)F)cc23)CC(C)N1] | |
| 216 | 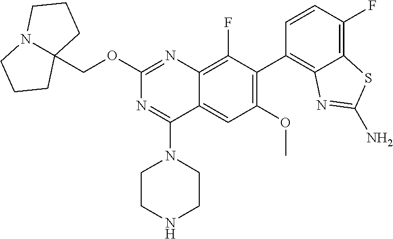 [SMILES: COc1cc2c(N3CCNCC3)nc(OCC34CCCN3CCC4)nc2c(F)c1-c1ccc(F)c2sc(N)nc12] | LCMS m/z [M + 1]: 590.29 ¹H NMR (300 MHz, CD₃OD) δ 7.28 (dd, J = 8.4, 5.4 Hz, 1H), 7.18 (s, 1H), 7.06 (t, J = 8.8 Hz, 1H), 4.67 (s, 2H), 4.23-4.07 (m, 4H), 3.88 (s, 3H), 3.69 (dt, J = 11.7, 5.7 Hz, 2H), 3.51 (t, J = 6.7 Hz, 4H), 3.30-3.23 (m, 2H), 2.41-2.05 (m, 8H). |
| 218 | 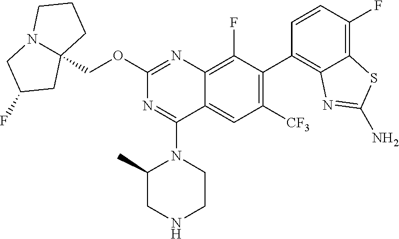 [SMILES: C[C@@H]1CNCCN1c1nc(OC[C@@]23CCCN2C[C@@H](F)C3)nc2c(F)c(-c3ccc(F)c4sc(N)nc34)c(C(F)(F)F)cc12] | LCMS m/z [M + 1]: 638.21 ¹HNMR (400 MHz, CD₃OD) δ 8.15 (s, 1H), 7.17-7.25 (m, 1H), 6.99 (t, J = 8.8 Hz, 1H), 5.50 (d, J = 51.2 Hz, 1H), 4.96-5.50 (m, 2H), 4.70-4.83 (m, 2H), 4.43-4.56 (m, 1H), 3.98- 4.09 (m, 1H), 3.88-3.98 (m, 1H), 3.60-3.71 (m, 1H), 3.39-3.56 (m, 5H), 2.64-2.77 (m, 1H), 2.38-2.64 (m, 2H), 2.29-2.32 (m, 2H), 2.11-2.20 (m, 1H), 1.66 (d, J = 6.8 Hz, 3H). |
| 219 | 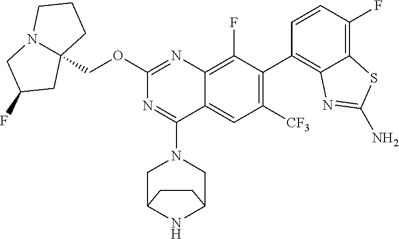 [SMILES: Nc1nc2c(-c3c(C(F)(F)F)cc4c(N5CC6CCC(C5)N6)nc(OC[C@@]56CCCN5C[C@H](F)C6)nc4c3F)ccc(F)c2s1] | LCMS m/z [M + 1]: 650.2 ¹HNMR (400 MHz, CD₃OD) δ 8.18 (s, 1H), 7.17-7.27 (m, 1H), 6.98-7.09 (m, 1H), 5.38-5.67 (m, 1H), 4.65-4.77 (m, 3H), 4.22-4.31 (m, 2H), 3.82-4.10 (m, 6H), 3.45-3.53 (m, 1H), 2.58-2.75 (m, 2H), 2.32-2.47 (m, 3H), 2.05-2.22 (m, 5H). |
| 219a | 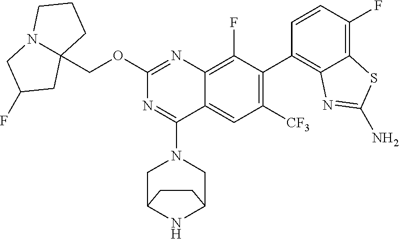 [SMILES: Nc1nc2c(-c3c(C(F)(F)F)cc4c(N5CC6CCC(C5)N6)nc(OCC56CCCN5CC(F)C6)nc4c3F)ccc(F)c2s1] | |
| 220 | 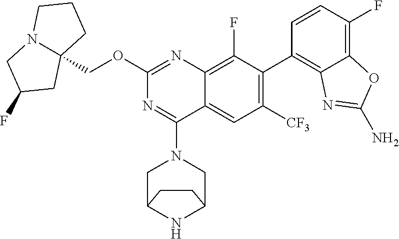 [SMILES: Nc1nc2c(-c3c(C(F)(F)F)cc4c(N5CC6CCC(C5)N6)nc(OC[C@@]56CCCN5C[C@H](F)C6)nc4c3F)ccc(F)c2o1] | LCMS m/z [M + 1]: 634.2 ¹HNMR (400 MHz, CD₃OD) δ 8.17 (s, 1H), 7.05-7.12(m, 1H), 6.96-7.03 (m, 1H), 5.50- 5.64 (m, 1H), 4.67-4.82 (m, 3H), 4.23- 4.31 (m, 2H), 3.83-4.09 (m, 6H), 3.45- 3.55 (m, 1H), 2.56-2.77 (m, 2H), 2.32- 2.45 (m, 3H), 2.07-2.23 (m, 5H). |
| 220a | 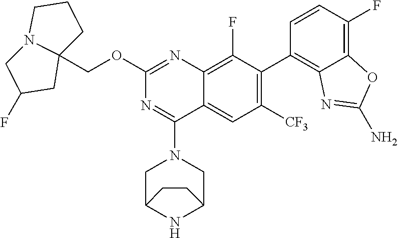 [SMILES: Nc1nc2c(-c3c(C(F)(F)F)cc4c(N5CC6CCC(C5)N6)nc(OCC56CCCN5CC(F)C6)nc4c3F)ccc(F)c2o1] | |
| 221 | 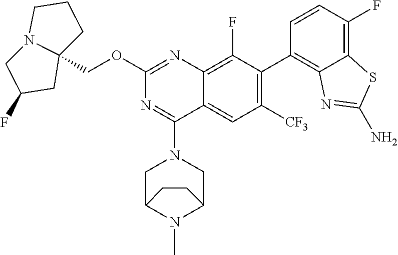 [SMILES: CN1C2CCC1CN(c1nc(OC[C@@]34CCCN3C[C@H](F)C4)nc3c(F)c(-c4ccc(F)c5sc(N)nc45)c(C(F)(F)F)cc13)C2] | LCMS m/z [M + 1]: 664.2 ¹HNMR (400 MHz, CD₃OD) δ 8.17 (s, 1H), 7.19-7.28 (m, 1H), 7.02 (t, J = 8.8 Hz, 1H), 5.50-5.65 (m, 1H), 4.67-4.72 (m, 2H), 4.11-4.19 (m, 2H), 3.99-4.08 (m, 3H), 3.85-3.96 (m, 3H), 3.43-3.52 (m, 2H), 2.87-2.98 (m, 3H), 2.58-2.79 (m, 2H), 2.30-2.47 (m, 5H), 2.08-2.21 (m, 3H). |
| 221a | 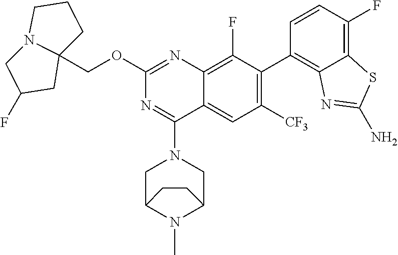 [SMILES: CN1C2CCC1CN(c1nc(OCC34CCCN3CC(F)C4)nc3c(F)c(-c4ccc(F)c5sc(N)nc45)c(C(F)(F)F)cc13)C2] | |
| 222 | 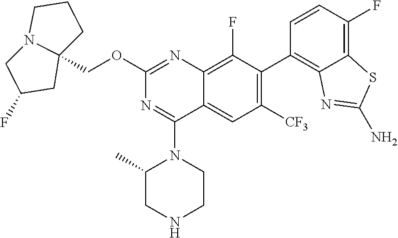 [SMILES: C[C@H]1CNCCN1c1nc(OC[C@@]23CCCN2C[C@@H](F)C3)nc2c(F)c(-c3ccc(F)c4sc(N)nc34)c(C(F)(F)F)cc12] | LCMS m/z [M + 1]: 638.2 ¹HNMR (400 MHz, CD₃OD) δ 8.15 (s, 1H), 7.19-7.23 (m, 1H), 7.02 (t, J = 8 Hz, 1H), 5.44-5.57 (m, 1H), 4.79-5.00 (m, 2H), 4.76- 4.79 (m, 2H), 4.48-4.52 (m, 1H), 3.93-4.04 (m, 2H), 3.62-3.67 (m, 2H), 3.40-3.45 (m, 4H), 2.65-2.80 (m, 1H), 2.42-2.45 (m, 2H), 2.23-2.32 (m, 2H), 2.14-2.173 (m, 1H), 1.66 (d, J = 6.8 Hz, 3H). |
| 223 | 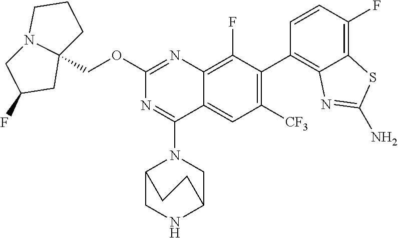 [SMILES: Nc1nc2c(-c3c(C(F)(F)F)cc4c(N5CC6CCC5CN6)nc(OC[C@@]56CCCN5C[C@H](F)C6)nc4c3F)ccc(F)c2s1] | LCMS m/z [M + 1]: 650.2 ¹HNMR (400 MHz, CD₃OD) 8.32(s, 1H), 7.12-7.20(m, 1H), 6.98-7.03(m, 1H), 5.49- 5.62(m, 1H), 5.18(s, 1H), 4.67-4.69(m, 2H), 4.52-4.55(m, 1H), 4.39-4.42(m, 1H), 3.79- 3.99(m, 6H), 3.57-3.63(m, 1H), 3.43-3.48(m, 1H), 2.05-2.71(m, 10H). |
| 223a | 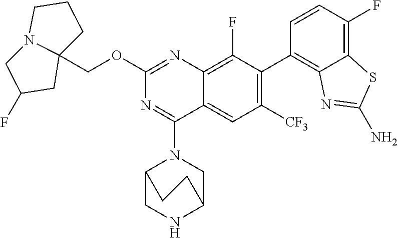 [SMILES: Nc1nc2c(-c3c(C(F)(F)F)cc4c(N5CC6CCC5CN6)nc(OCC56CCCN5CC(F)C6)nc4c3F)ccc(F)c2s1] | |
| 224 | 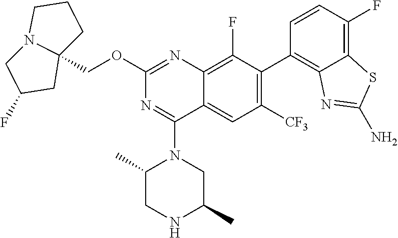 [SMILES: C[C@@H]1CN(c2nc(OC[C@@]34CCCN3C[C@@H](F)C4)nc3c(F)c(-c4ccc(F)c5sc(N)nc45)c(C(F)(F)F)cc23)[C@@H](C)CN1] | LCMS m/z [M + 1]: 652.2 ¹HNMR (400 MHz, CD₃OD): δ 8.24 (s, 1H), 7.18-7.24 (m, 1H), 7.00 (t, J = 8.8 Hz, 1H), 5.51 (d, J = 50.8 Hz, 1H), 4.62-4.71 (m, 2H), 4.02-4.16 (m, 3H), 3.81-3.92 (m, 1H), 3.60- 3.72 (m, 2H), 3.56-3.62 (m, 1H), 3.44-3.51 (m, 3H), 2.64-2.78 (m, 1H), 2.39-2.53 (m, 2H), 2.22-2.33 (m, 2H), 2.16-2.19 (m, 1H), 1.59 (d, J = 6.4 Hz, 3H), 1.45 (d, J = 6.4 Hz, 3H). |
| 225 | 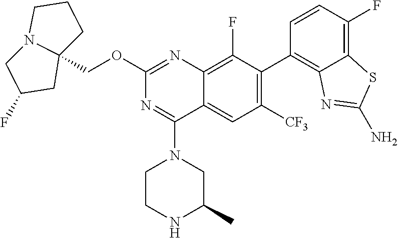 [SMILES: C[C@@H]1CN(c2nc(OC[C@@]34CCCN3C[C@@H](F)C4)nc3c(F)c(-c4ccc(F)c5sc(N)nc45)c(C(F)(F)F)cc23)CCN1] | LCMS m/z [M + 1]: 638.2 ¹HNMR (400 MHz, CD₃OD) δ 8.20 (s, 1H), 7.15-7.25 (m, 1H), 7.00 (t, J = 8.8 Hz, 1H), 5.54 (d, J = 51.2 Hz, 1H), 4.66-4.82 (m, 2H), 4.49-4.66 (m, 2H), 3.99-4.21 (m, 1H), 3.75- 3.99 (m, 2H), 3.40-3.72 (m, 6H), 2.11-2.79 (m, 6H), 1.44 (d, J = 6.4 Hz, 3H). |
| 226 | 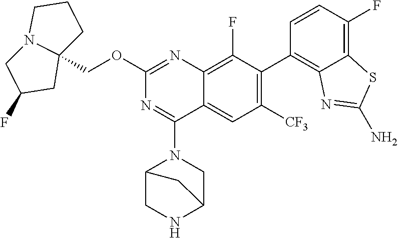 [SMILES: Nc1nc2c(-c3c(C(F)(F)F)cc4c(N5CC6CC5CN6)nc(OC[C@@]56CCCN5C[C@H](F)C6)nc4c3F)ccc(F)c2s1] | LCMS m/z [M + 1]: 636.3 ¹HNMR (400 MHz, CD₃OD) δ 8.29 (s, 1H), 7.20 (d, J = 5.6 Hz, 1H), 7.0 (t, J = 9.2 Hz, 1H), 5.50-5.70 (m, 2H), 4.55-4.80 (m, 4H), 4.22 (d, J = 10.8 Hz, 1H), 3.86-4.10 (m, 3H), 3.40- 3.70 (m, 3H), 2.58-2.74 (m, 2H), 2.10-2.45 (m, 6H). |
| 226a | 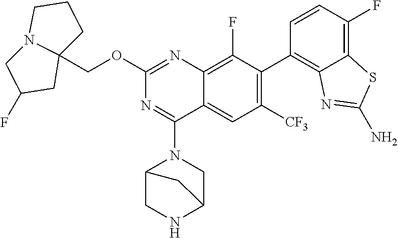 [SMILES: Nc1nc2c(-c3c(C(F)(F)F)cc4c(N5CC6CC5CN6)nc(OCC56CCCN5CC(F)C6)nc4c3F)ccc(F)c2s1] | |
| 227 | 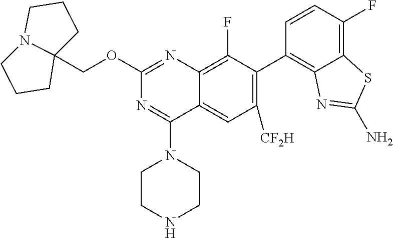 [SMILES: Nc1nc2c(-c3c(C(F)F)cc4c(N5CCNCC5)nc(OCC56CCCN5CCC6)nc4c3F)ccc(F)c2s1] | LCMS m/z [M + 1]: 588.09 ¹HNMR (300 MHz, CD₃OD) δ 8.13 (s, 1H), 7.45-7.20 (m, 1H), 7.09 (t, J = 8.7 Hz, 1H), 6.64 (t, J = 55.0 Hz, 1H), 4.72 (s, 2H), 4.22 (m, 4H), 3.71 (m, 2H), 3.52 (m, 4H), 3.31- 3.23 (m, 2H), 2.49-2.08 (m, 8H). |
| 228 | 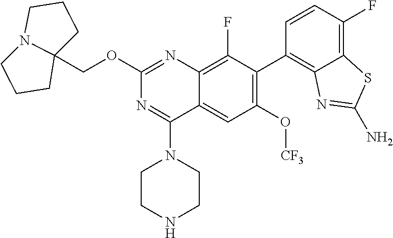 [SMILES: Nc1nc2c(-c3c(OC(F)(F)F)cc4c(N5CCNCC5)nc(OCC56CCCN5CCC6)nc4c3F)ccc(F)c2s1] | LCMS m/z [M + 1]: 622.36 ¹HNMR (300 MHz, CD₃OD) δ 7.68 (s, 1H), 7.24 (dd, J = 8.4, 5.1 Hz, 1H), 7.01 (t, J = 8.8 Hz, 1H), 4.54 (s, 2H), 4.08-3.96 (m, 4H), 3.63-3.49 (m, 2H), 3.41-3.34 (m, 2H), 3.22-3.07 (m, 4H), 2.20-1.91 (m, 8H). |
| 229 | 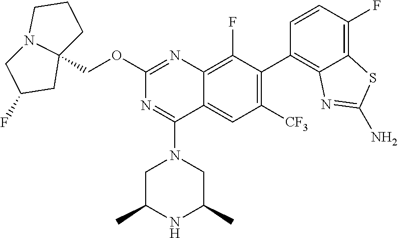 [SMILES: C[C@@H]1CN(c2nc(OC[C@@]34CCCN3C[C@@H](F)C4)nc3c(F)c(-c4ccc(F)c5sc(N)nc45)c(C(F)(F)F)cc23)C[C@H](C)N1] | LCMS m/z [M + 1]: 652.2 ¹HNMR (400 MHz, CD₃OD) δ 8.19 (s, 1H), 7.20-7.21 (m, 1H), 7.00 (t, J = 8.8 Hz, 1H), 5.44-5.57 (m, 1H), 4.77-4.79 (m, 2H), 4.62- 4.66 (m, 2H), 4.01-4.09 (m, 1H), 3.45-3.67 (m, 6H), 2.67-2.72 (m, 1H), 2.14-2.53 (m, 6H), 1.44 (s, 3H), 1.42 (s, 3H). |
| 230 | 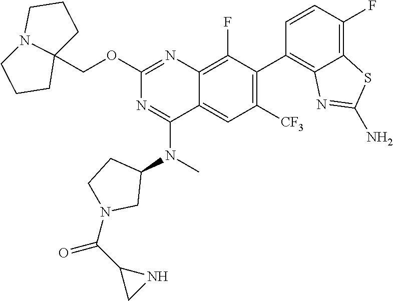 [SMILES: CN(c1nc(OCC23CCCN2CCC3)nc2c(F)c(-c3ccc(F)c4sc(N)nc34)c(C(F)(F)F)cc12)[C@@H]1CCN(C(=O)C2CN2)C1] | LCMS m/z [M + 1]: 689.3 ¹HNMR (400 MHz, CD₃OD) δ 8.37-8.40 (m, 1H), 7.29-7.35 (m, 1H), 7.17-7.25 (m, 1H), 6.97 (t, J = 8.8 Hz, 1H), 5.25-5.52 (m, 1H), 4.58 (s, 2H), 3.80-4.23 (m, 4H), 3.48-3.69 (m, 7H), 3.09-3.21 (m, 2H), 2.79-2.91 (m, 1H), 1.85-2.63 (m, 10H). |
| 230a | 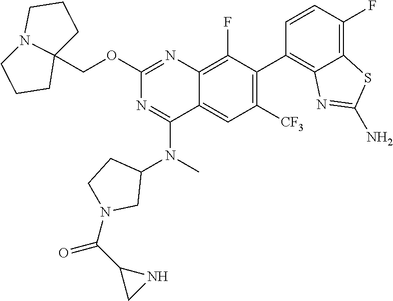 [SMILES: CN(c1nc(OCC23CCCN2CCC3)nc2c(F)c(-c3ccc(F)c4sc(N)nc34)c(C(F)(F)F)cc12)C1CCN(C(=O)C2CN2)C1] | |
| 231 | 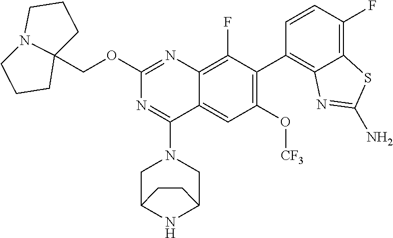 [SMILES: Nc1nc2c(-c3c(OC(F)(F)F)cc4c(N5CC6CCC(C5)N6)nc(OCC56CCCN5CCC6)nc4c3F)ccc(F)c2s1] | LCMS m/z [M + 1]: 648.08 1H NMR (300 MHz, CD₃OD) δ 7.79 (s, 1H), 7.30 (dd, J = 8.5, 5.5 Hz, 1H), 7.07 (t, J = 8.8 Hz, 1H), 4.77-4.62 (m, 4H), 4.28 (s, 2H), 4.00-3.88 (m, 2H), 3.83-3.64 (m, 2H), 3.46-3.35 (m, 2H), 2.41-2.03 (m, 12H). |
| 232 | 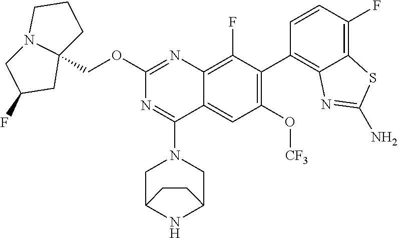 [SMILES: Nc1nc2c(-c3c(OC(F)(F)F)cc4c(N5CC6CCC(C5)N6)nc(OC[C@@]56CCCN5C[C@H](F)C6)nc4c3F)ccc(F)c2s1] | LCMS m/z [M + 1]: 666.2 ¹HNMR (400 MHz, CD₃OD) δ 7.77 (s, 1H), 7.24-7.27 (m, 1H), 7.02 (t, J = 8.0 Hz, 1H), 5.51-5.63 (m, 1H), 4.61-4.73 (m, 4H), 4.25 (s, 2H), 3.88-4.04 (m, 5H), 3.44-3.51 (m, 1H), 2.32-2.75 (m, 5H), 2.10-2.17 (m, 5H). |
| 232a | 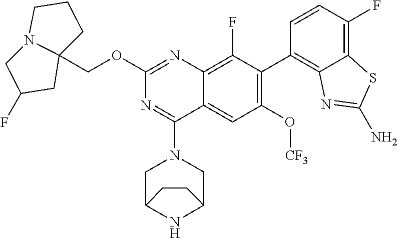 [SMILES: Nc1nc2c(-c3c(OC(F)(F)F)cc4c(N5CC6CCC(C5)N6)nc(OCC56CCCN5CC(F)C6)nc4c3F)ccc(F)c2s1] | |
| 233 | 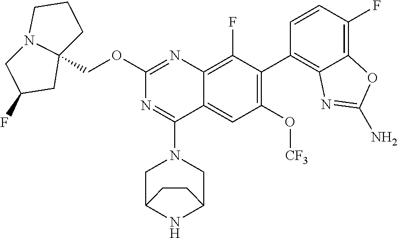 [SMILES: Nc1nc2c(-c3c(OC(F)(F)F)cc4c(N5CC6CCC(C5)N6)nc(OC[C@@]56CCCN5C[C@H](F)C6)nc4c3F)ccc(F)c2o1] | LCMS m/z [M + 1]: 650.2 |
| 233a | 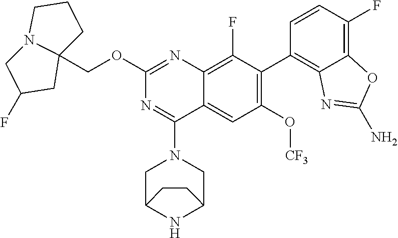 [SMILES: Nc1nc2c(-c3c(OC(F)(F)F)cc4c(N5CC6CCC(C5)N6)nc(OCC56CCCN5CC(F)C6)nc4c3F)ccc(F)c2o1] | |
| 234 | 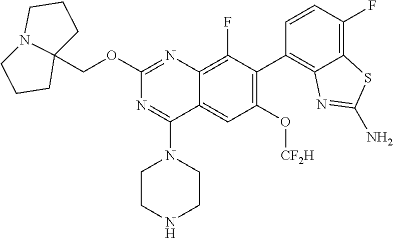 [SMILES: Nc1nc2c(-c3c(OC(F)F)cc4c(N5CCNCC5)nc(OCC56CCCN5CCC6)nc4c3F)ccc(F)c2s1] | LCMS m/z [M + 1]: 650.2 ¹HNMR (400 MHz, CD₃OD) δ 7.78 (s, 1H), 7.14-7.16 (m, 1H), 7.02 (t, J = 8.0 Hz, 1H), 5.50-5.63 (m, 1H), 4.61-4.73 (m, 4H), 4.25 (s, 2H), 3.88-4.04 (m, 5H), 3.44-3.51 (m, 1H), 2.32-2.75 (m, 5H), 2.10-2.17 (m, 5H). |
| 235 | 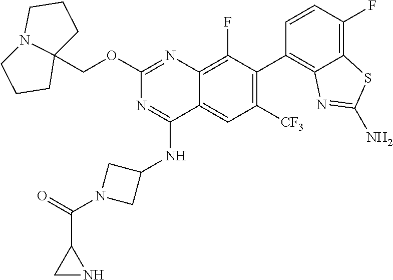 [SMILES: Nc1nc2c(-c3c(C(F)(F)F)cc4c(NC5CN(C(=O)C6CN6)C5)nc(OCC56CCCN5CCC6)nc4c3F)ccc(F)c2s1] | LCMS m/z [M + 1]: 661.2 ¹HNMR (400 MHz, CD₃OD) δ 8.61 (s, 1H), 7.16-7.25 (m, 1H), 6.98 (t, J = 8.8 Hz, 1H), 4.96-5.21 (m, 2H), 4.66 (s, 2H), 4.46-4.61 (m, 2H), 4.15-4.34 (m, 1H), 3.76-3.98 (m, 1H), 3.64-3.74 (m, 2H), 3.20-3.29 (m, 2H), 2.46-2.60 (m, 1H), 2.28-2.38 (m, 2H), 2.19- 2.25 (m, 2H), 2.08-2.18 (m, 4H), 2.03 (s, 1H). |
| 236 | 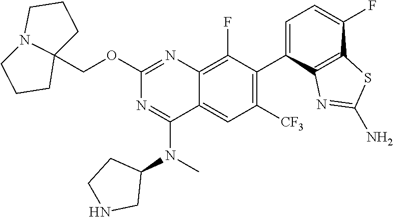 | LCMS m/z [M + 1]: 620.3 ¹HNMR (400 MHz, CD₃OD) δ 8.37 (s, 1H), 7.17-7.24 (m, 1H), 6.99 (t, J = 8.9 Hz, 1H), 5.26-5.36 (m, 1H), 4.68 (s, 2H), 3.80-3.80 (m, 1H), 3.62-3.76 (m, 4H), 3.54 (s, 3H), 3.38-3.51 (m, 3H), 2.51-2.62 (m, 1H), 2.30- 2.46 (m, 3H), 2.20-2.26 (m, 2H), 2.07-2.16 (m, 4H). |
| 236a | 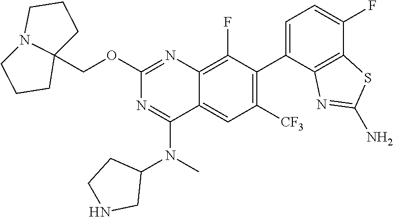 [SMILES: CN(c1nc(OCC23CCCN2CCC3)nc2c(F)c(-c3ccc(F)c4sc(N)nc34)c(C(F)(F)F)cc12)C1CCNC1] | |
| 237 | 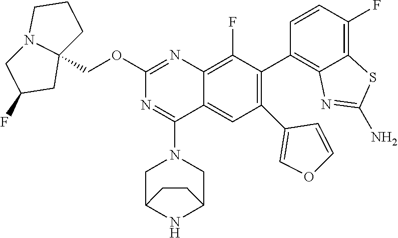 [SMILES: Nc1nc2c(-c3c(-c4ccoc4)cc4c(N5CC6CCC(C5)N6)nc(OC[C@@]56CCCN5C[C@H](F)C6)nc4c3F)ccc(F)c2s1] | LCMS m/z [M + 1]: 648.2 ¹HNMR (400 MHz, CD₃OD) δ 7.87 (s, 1H), 7.33 (t, J = 1.6 Hz, 1H), 7.19 (s, 1H), 7.12 (dd, J = 8.4, 5.4 Hz, 1H), 6.98 (t, J = 8.8 Hz, 1H), 6.14 (s, 1H), 5.57 (d, J = 51.8 Hz, 1H), 4.76-4.84 (m, 3H), 4.68-4.73 (m, 2H), 4.25 (s, 2H), 3.86-3.99 (m, 4H), 3.42-3.51 (m, 1H), 2.58-2.77 (m, 2H), 2.31-2.48 (m, 3H), 2.08-2.22 (m, 5H). |
| 237a | 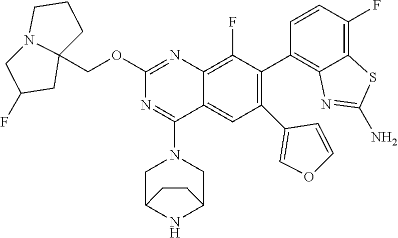 [SMILES: Nc1nc2c(-c3c(-c4ccoc4)cc4c(N5CC6CCC(C5)N6)nc(OCC56CCCN5CC(F)C6)nc4c3F)ccc(F)c2s1] | |
| 238 | 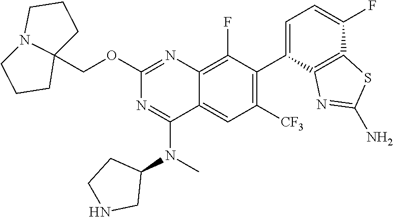 | LCMS m/z [M + 1]: 620.3 ¹HNMR (400 MHz, CD₃OD) δ 8.37 (s, 1H), 7.18-7.25 (m, 1H), 6.99 (t, J = 8.8 Hz, 1H), 5.26-5.36 (m, 1H), 4.67 (s, 2H), 3.85 (dd, J = 12.1, 8.9 Hz, 1H), 3.64-3.73 (m, 3H), 3.54 (s, 3H), 3.38-3.50 (m, 3H), 3.25-3.25 (m, 1H), 2.50-2.63 (m, 1H), 2.08-2.47 (m, 10H). |
| 239 | 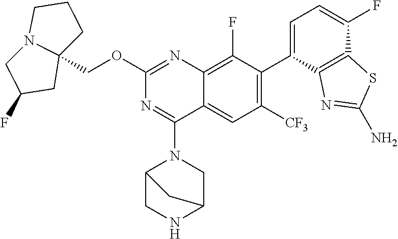 | LCMS m/z [M + 1]: 636.3 ¹HNMR (400 MHz, CD₃OD) δ 8.27(s, 1H), 7.10-7.26 (m, 1H), 7.01 (t, J = 8.8 Hz, 1H), 5.54 (s, 1H), 5.51 (d, J = 51.6 Hz, 1H), 4.55- 4.80 (m, 4H), 4.22 (d, J = 11.2 Hz 1H), 3.80- 4.04 (m, 4H), 3.31-3.57 (m, 3H), 2.60-2.78 (m, 2H), 2.10-2.47 (m, 6H). |
| 239a | 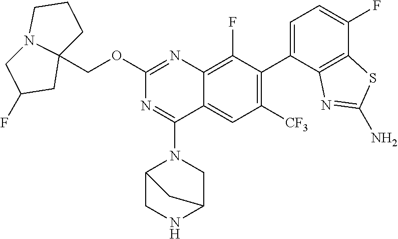 [SMILES: Nc1nc2c(-c3c(C(F)(F)F)cc4c(N5CC6CC5CN6)nc(OCC56CCCN5CC(F)C6)nc4c3F)ccc(F)c2s1] | |
| 240 | 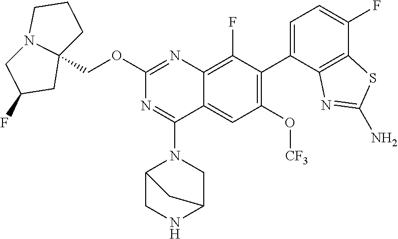 [SMILES: Nc1nc2c(-c3c(OC(F)(F)F)cc4c(N5CC6CC5CN6)nc(OC[C@@]56CCCN5C[C@H](F)C6)nc4c3F)ccc(F)c2s1] | LCMS m/z [M + 1]: 652.3 ¹HNMR (400 MHz, CD₃OD) δ 7.89 (s, 1H), 7.21-7.27 (m, 1H), 7.01 (t, J = 8.0 Hz, 1H), 5.47-5.60 (m, 2H), 4.61-4.66 (m, 3H), 4.48- 4.51 (m, 1H), 3.43-4.16 (m, 7H), 2.03-2.70 (m, 8H). |
| 240a | 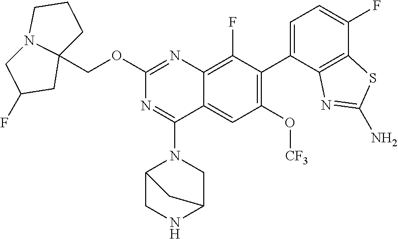 [SMILES: Nc1nc2c(-c3c(OC(F)(F)F)cc4c(N5CC6CC5CN6)nc(OCC56CCCN5CC(F)C6)nc4c3F)ccc(F)c2s1] | |
| 241 | 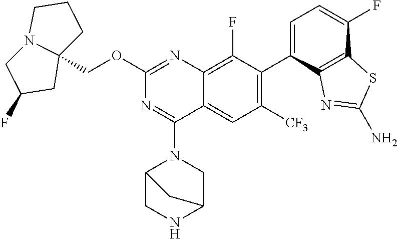 | LCMS m/z [M − 1]: 636.2 ¹HNMR (400 MHz, CD₃OD) δ 8.29 (s, 1H), 7.20 (d, J = 5.6 Hz 1H), 7.0 (t, J = 9.2 Hz 1H), 5.50-5.70 (m, 2H), 4.55-4.80 (m, 4H), 4.22 (d, J = 10.8 Hz 1H), 3.86-4.10 (m, 3H), 3.40- 3.70 (m, 3H), 2.58-2.74 (m, 2H), 2.10-2.45 (m, 6H). |
| 245 | 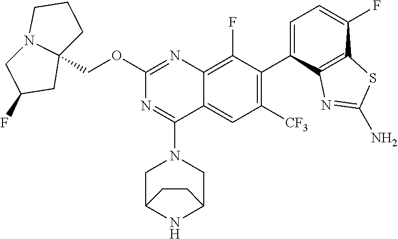 | LCMS m/z [M + 1]: 650.3 ¹HNMR (400 MHz, CD₃OD) δ 8.14 (s, 1H), 7.17-7.20 (m, 1H), 6.94-6.99 (m, 1H), 5.32 and 5.45 (m, 1H), 4.59-4.84 (m, 2H), 4.36- 4.55 (m, 2H), 3.73-3.84 (m, 4H), 3.39-3.76 (m, 3H), 3.14-3.20 (m, 1H), 1.87-2.25 (m, 10H). |
| 246 | 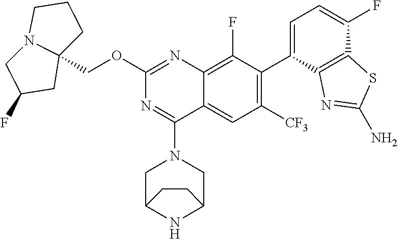 | LCMS m/z [M + 1]: 650.3 ¹HNMR (400 MHz, CD₃OD) δ 8.16 (s, 1H), 7.17-7.21 (m, 1H), 6.96-7.00 (m, 1H), 5.43 and 5.56 (m, 1H), 4.62-4.74 (m, 2H), 4.54- 4.59 (m, 2H), 4.09 (bs, 2H), 3.83-3.91 (m, 2H), 3.66-3.73 (m, 2H), 3.35 (m, 2H), 1.87- 2.25 (m, 10H). |
| 247 | 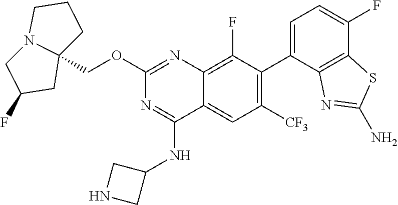 [SMILES: Nc1nc2c(-c3c(C(F)(F)F)cc4c(NC5CNC5)nc(OC[C@@]56CCCN5C[C@H](F)C6)nc4c3F)ccc(F)c2s1] | LCMS m/z [M − 1]: 608.2 ¹HNMR (400 MHz, CD₃OD): δ 8.57 (s, 1H), 7.19-7.30 (m, 1H), 6.98(t, J = 9.2 Hz, 1H), 5.54 (d, J = 52.2 Hz, 1H), 5.18-5.31 (m, 1H), 4.67-4.73 (m, 2H), 4.52 (t, J = 8.4 Hz, 2H), 4.37-4.49 (m, 2H), 3.90-4.05 (m, 3H), 3.40- 3.53 (m, 1H), 2.57-2.72 (m, 2H), 2.30-2.45 (m, 3H), 2.10-2.18 (m, 1H). |
| 247a | 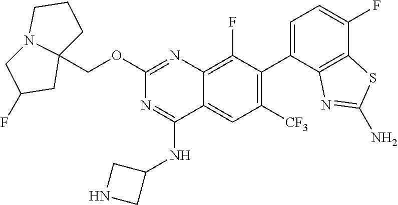 [SMILES: Nc1nc2c(-c3c(C(F)(F)F)cc4c(NC5CNC5)nc(OCC56CCCN5CC(F)C6)nc4c3F)ccc(F)c2s1] | |
| 248 | 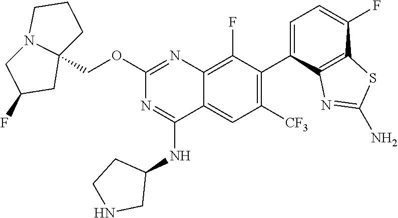 | LCMS m/z [M + 1]: 624.2 ¹HNMR (400 MHz, CD₃OD) δ 8.61 (s, 1H), 7.16-7.20 (m, 1H), 6.98 (t, J = 8.8 Hz, 1H), 5.55 (d, J = 52 Hz, 1H), 5.00-5.03 (m, 2H), 4.70-4.72 (m, 2H), 3.80-3.92 (m, 4H), 3.62- 3.65 (m, 1H), 3.45-3.52 (m, 3H), 2.53-2.63 (m, 3H), 2.32-2.44 (m, 4H), 2.15(m, 1H). |
| 248a | 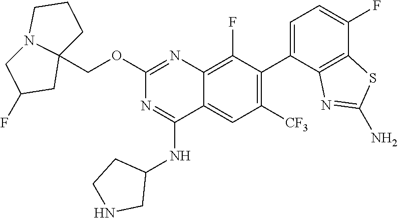 [SMILES: Nc1nc2c(-c3c(C(F)(F)F)cc4c(NC5CCNC5)nc(OCC56CCCN5CC(F)C6)nc4c3F)ccc(F)c2s1] | |
| 249 | 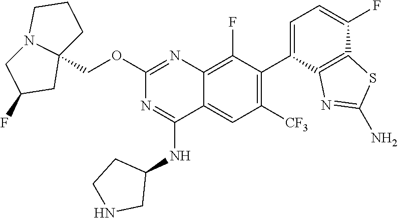 | LCMS m/z [M + 1]: 624.2 ¹HNMR (400 MHz, CD₃OD) δ 8.61 (s, 1H), 7.17-7.21 (m, 1H), 6.97 (t, J = 8.8 Hz, 1H), 5.55 (d, J = 51.6 Hz, 1H), 5.00-5.03 (m, 2H), 4.70-4.71(m, 2H), 3.81-3.92 (m, 4H), 3.61-3.64 (m, 1H), 3.44-3.52 (m, 3H), 2.52- 2.69 (m, 3H), 2.30-2.43 (m, 4H), 2.15 (m, 1H). |
| 250 | 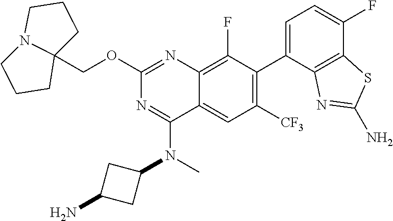 | LCMS m/z [M + 1]: 620.3 ¹HNMR (400 MHz, CD₃OD) δ 8.27 (s, 1H), 7.22-7.25 (m, 1H), 7.02 (t, J = 8.8 Hz, 1H), 4.64-4.66 (m, 3H), 3.67-3.69 (m, 3H), 3.48 (s, 3H),3.27-3.29 (m, 2H), 2.92-2.95 (m, 2H), 2.50-2.52 (m, 2H), 2.15-2.34 (m, 8H) |
| 254 | 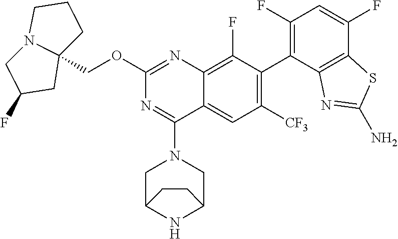 [SMILES: Nc1nc2c(-c3c(C(F)(F)F)cc4c(N5CC6CCC(C5)N6)nc(OC[C@@]56CCCN5C[C@H](F)C6)nc4c3F)c(F)cc(F)c2s1] | |
| 254a | 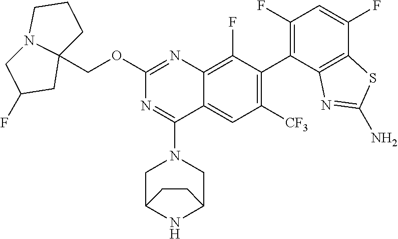 [SMILES: Nc1nc2c(-c3c(C(F)(F)F)cc4c(N5CC6CCC(C5)N6)nc(OCC56CCCN5CC(F)C6)nc4c3F)c(F)cc(F)c2s1] | |
| 255 | 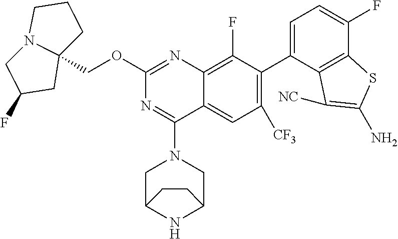 [SMILES: N#Cc1c(N)sc2c(F)ccc(-c3c(C(F)(F)F)cc4c(N5CC6CCC(C5)N6)nc(OC[C@@]56CCCN5C[C@H](F)C6)nc4c3F)c12] | LCMS m/z [M + 1]: 674.4 ¹HNMR (400 MHz, CD₃OD) δ 8.17 (s, 1H), 7.22 (dd, J = 8.4, 5.2 Hz, 1H), 7.03 (t, J = 8.8 Hz, 1H), 5.57 (d, J = 52 Hz, 1H), 4.64-4.83 (m, 4H), 4.25 (s, 2H), 3.84-4.08 (m, 5H), 3.43-3.53 (m, 1H), 2.58-2.80 (m, 2H), 2.30- 2.50 (m, 3H), 2.03-2.24 (m, 5H). |
| 255a | 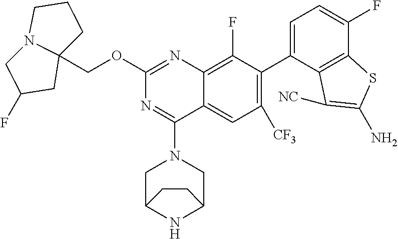 [SMILES: N#Cc1c(N)sc2c(F)ccc(-c3c(C(F)(F)F)cc4c(N5CC6CCC(C5)N6)nc(OCC56CCCN5CC(F)C6)nc4c3F)c12] | |
| 256 | 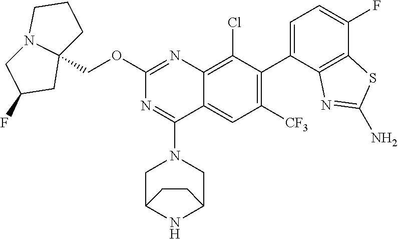 [SMILES: Nc1nc2c(-c3c(C(F)(F)F)cc4c(N5CC6CCC(C5)N6)nc(OC[C@@]56CCCN5C[C@H](F)C6)nc4c3Cl)ccc(F)c2s1] | LCMS m/z (M + H): 666.3 ¹HNMR (400 MHz, CD₃OD) δ 8.30 (s, 1H), 7.07-7.18 (m, 1H), 7.00 (t, J = 8.8 Hz, 1H), 5.56(d, J = 52.8 Hz, 1H), 4.60-4.83 (m, 3H), 4.25 (s, 2H), 3.85-4.10 (m, 5H), 3.41-3.52 (m, 2H), 2.56-2.80 (m, 2H), 2.30-2.52 (m, 3H), 1.83-2.23 (m, 5H). |
| 256a | 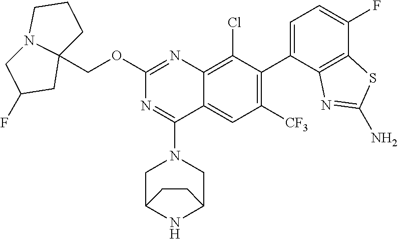 [SMILES: Nc1nc2c(-c3c(C(F)(F)F)cc4c(N5CC6CCC(C5)N6)nc(OCC56CCCN5CC(F)C6)nc4c3Cl)ccc(F)c2s1] | |
| 257 | 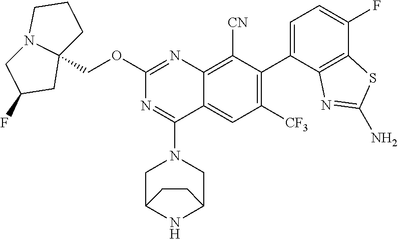 [SMILES: N#Cc1c(-c2ccc(F)c3sc(N)nc23)c(C(F)(F)F)cc2c(N3CC4CCC(C3)N4)nc(OC[C@@]34CCCN3C[C@H](F)C4)nc12] | |
| 257a | 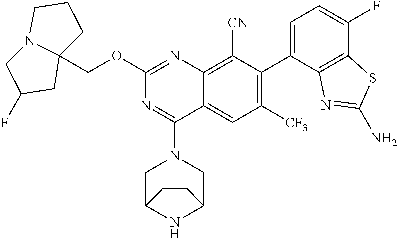 [SMILES: N#Cc1c(-c2ccc(F)c3sc(N)nc23)c(C(F)(F)F)cc2c(N3CC4CCC(C3)N4)nc(OCC34CCCN3CC(F)C4)nc12] | |
| 258 | 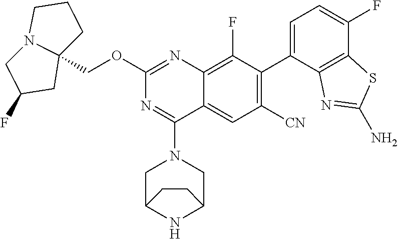 [SMILES: N#Cc1cc2c(N3CC4CCC(C3)N4)nc(OC[C@@]34CCCN3C[C@H](F)C4)nc2c(F)c1-c1ccc(F)c2sc(N)nc12] | LCMS m/z [M + 1]: 607.1 ¹HNMR (400 MHz, CD₃OD) δ 8.34 (s, 1H), 7.39-7.36 (m, 1H), 7.07 (t, 1H), 5.66 (s, 0.5H), 5.53 (s, 0.5H), 4.91-4.76 (m, 3H), 4.34- 4.19 (m, 2H), 4.09-3.84 (m, 4H), 3.55-3.44 (m, 1H), 2.84-2.55 (m, 2H), 2.50-2.28 (m, 3H), 2.24-1.97 (m, 5H), 1.40-1.32 (m, 2H). |
| 258a | 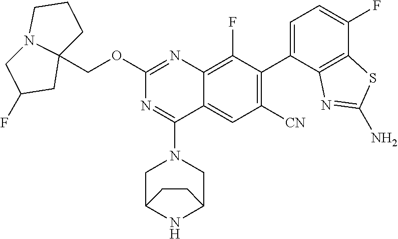 [SMILES: N#Cc1cc2c(N3CC4CCC(C3)N4)nc(OCC34CCCN3CC(F)C4)nc2c(F)c1-c1ccc(F)c2sc(N)nc12] | |
| 259 | 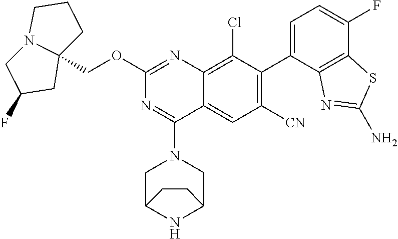 [SMILES: N#Cc1cc2c(N3CC4CCC(C3)N4)nc(OC[C@@]34CCCN3C[C@H](F)C4)nc2c(Cl)c1-c1ccc(F)c2sc(N)nc12] | |
| 259a | 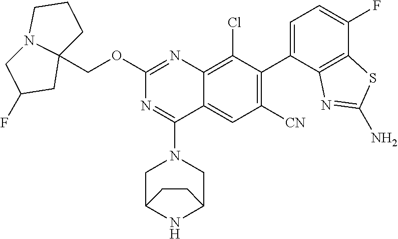 [SMILES: N#Cc1cc2c(N3CC4CCC(C3)N4)nc(OCC34CCCN3CC(F)C4)nc2c(Cl)c1-c1ccc(F)c2sc(N)nc12] | |
| 260 | 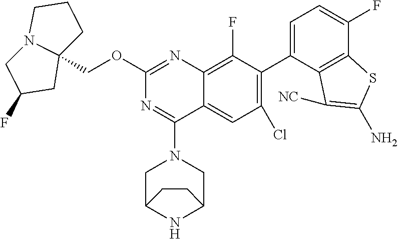 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12] | LCMS m/z [M + 1]: 640.35 ¹HNMR (300 MHz, CD₃OD) δ 7.96 (d, J = 1.7 Hz, 1H), 7.22 (dd, J = 8.4, 5.1 Hz, 1H), 7.06 (dd, J = 9.5, 8.4 Hz, 1H), 5.58 (d, J = 48.0 Hz, 1H), 4.77-4.62 (m, 4H), 4.24 (s, 2H), 4.01-3.86 (m, 5H), 3.48-3.41 (m, 1H), 2.74-2.54 (m, 2H), 2.43-2.27 (m, 3H), 2.22-2.10 (m, 5H). |
| 260a | 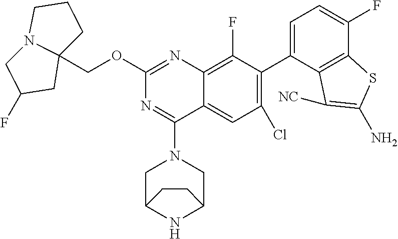 [SMILES: N#Cc1c(N)sc2c(F)ccc(-c3c(Cl)cc4c(N5CC6CCC(C5)N6)nc(OCC56CCCN5CC(F)C6)nc4c3F)c12] | |
| 261 | 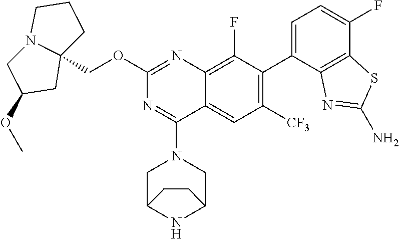 [SMILES: CO[C@H]1CN2CCC[C@@]2(COc2nc(N3CC4CCC(C3)N4)c3cc(C(F)(F)F)c(-c4ccc(F)c5sc(N)nc45)c(F)c3n2)C1] | LCMS m/z [M + 1]: 662.2 ¹HNMR(400 MHz, CD₃OD) δ 8.17 (s, 1H), 7.17-7.23 (m,1H), 6.99 (d, J = 8.8 Hz, 1H), 4.72-4.81 (m, 2H), 4.60-4.72 (m, 2H), 4.22- 4.33 (m, 3H), 3.93-4.02 (m, 2H), 3.73-3.82 (m, 2H), 3.61-3.68 (m, 1H), 3.46-3.57 (m, 1H), 3.39 (s, 3H), 2.24-2.50 (m, 4H), 2.03- 2.19 (m, 6H). |
| 261a | 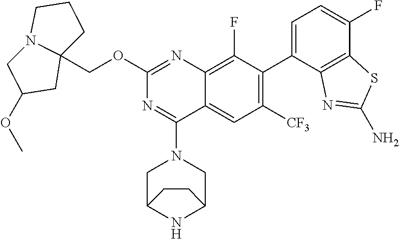 [SMILES: COC1CN2CCCC2(COc2nc(N3CC4CCC(C3)N4)c3cc(C(F)(F)F)c(-c4ccc(F)c5sc(N)nc45)c(F)c3n2)C1] | |
| 262 | 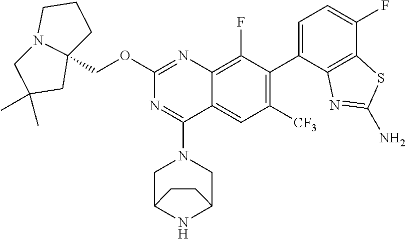 [SMILES: CC1(C)CN2CCC[C@@]2(COc2nc(N3CC4CCC(C3)N4)c3cc(C(F)(F)F)c(-c4ccc(F)c5sc(N)nc45)c(F)c3n2)C1] | |
| 262a | 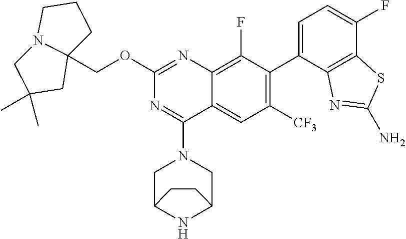 [SMILES: CC1(C)CN2CCCC2(COc2nc(N3CC4CCC(C3)N4)c3cc(C(F)(F)F)c(-c4ccc(F)c5sc(N)nc45)c(F)c3n2)C1] | |
| 263 | 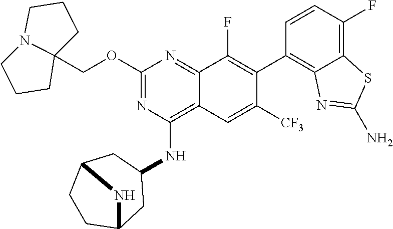 | LCMS m/z [M + 1]: 646.1 ¹H NMR (500 MHz, MeOD) δ 8.69 (s, 1H), 7.37-7.31 (m, 1H), 7.13 (t, J = 8.8 Hz, 1H), 4.88 (dq, J = 11.4, 5.7 Hz, 1H), 4.80 (d, J = 12.4 Hz, 1H), 4.74 (d, J = 12.4 Hz, 1H), 4.21 (q, J = 3.0 Hz, 2H), 3.71 (ddd, J = 11.8, 7.4, 6.0 Hz, 2H), 3.31-3.24 (m, 4H), 2.41-2.03 (m, 14H). |
| 264 | 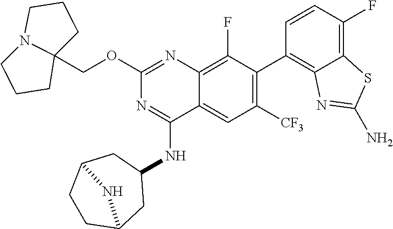 | LCMS m/z [M + 1]: 646.1 ¹H NMR (500 MHz, MeOD) δ 8.60 (s, 1H), 7.27 (dd, J = 8.6, 5.2 Hz, 1H), 7.10-7.02 (m, 1H), 4.69 (d, J = 4.8 Hz, 2H), 4.49 (t, J = 6.5 Hz, 1H), 4.12 (p, J = 3.2 Hz, 2H), 3.74- 3.64 (m, 2H), 3.28 (dd, J = 10.8, 6.6 Hz, 2H), 2.55 (d, J = 16.0 Hz, 2H), 2.47 (ddt, J = 16.2, 6.7, 3.4 Hz, 3H), 2.42-2.35 (m, 3H), 2.31 (ddd, J = 12.8, 9.2, 6.1 Hz, 3H), 2.23 (p, J = 5.4, 4.4 Hz, 5H), 2.17-2.06 (m, 4H). |
| 265 | 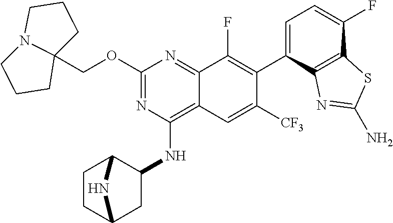 | LCMS m/z [M + 1]: 632.1 ¹H NMR (500 MHz, MeOD) δ 8.56 (s, 1H), 7.25-7.19 (m, 1H), 7.02 (dd, J = 9.1, 8.5 Hz, 1H), 4.75-4.65 (m, 2H), 4.55-4.48 (m, 2H), 4.41 (t, J = 4.4 Hz, 1H), 3.74-3.64 (m, 2H), 3.30-3.25 (m, 1H), 2.53-1.81 (m, 15H). |
| 266 | 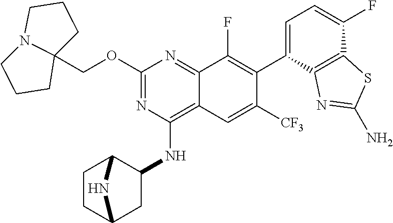 | LCMS m/z [M + 1]: 632.1 ¹H NMR (500 MHz, MeOD) δ 8.56 (d, J = 5.9 Hz, 1H), 7.23 (q, J = 6.9, 6.2 Hz, 1H), 7.03 (q, J = 8.3 Hz, 1H), 4.72-4.67 (m, 2H), 4.50 (ddt, J = 21.8, 7.7, 4.2 Hz, 2H), 4.40 (p, J = 4.2 Hz, 1H), 3.69 (dt, J = 13.2, 6.8 Hz, 2H), 3.27 (s, 1H), 2.58-1.80 (m, 15H). |
| 267 | 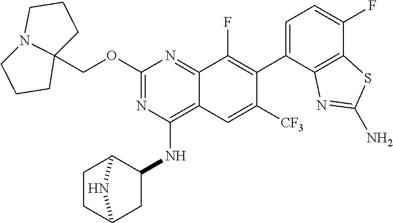 | |
| 268 | 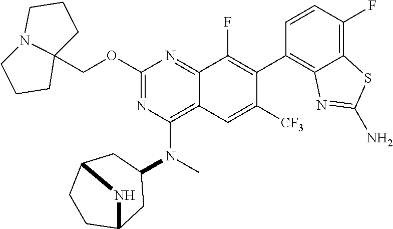 | LCMS m/z [M + 1]: 660.2 ¹H NMR (500 MHz, CD₃OD) δ 8.39 (s, 1H), 7.32 (td, J = 8.7, 5.4 Hz, 1H), 7.13-7.06 (m, 1H), 5.34 (tt, J = 11.8, 5.6 Hz, 1H), 4.73 (q, J = 12.1 Hz, 2H), 4.27 (p, J = 3.0 Hz, 2H), 3.77-3.60 (m, 2H), 3.49 (s, 3H), 3.34-3.22 (m, 3H), 2.43-1.96 (m, 15H). |
| 269 | 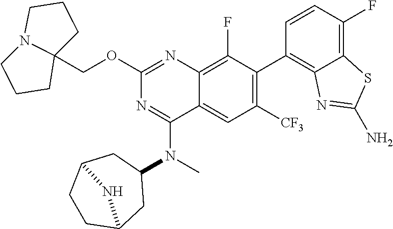 | |
| 270 | 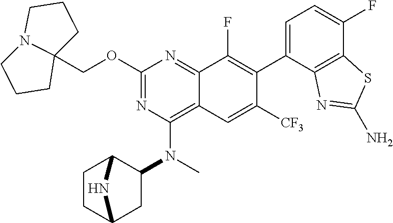 | LCMS m/z [M + 1]: 646.1 ¹HNMR (500 MHz, CD₃OD) δ 8.33 (d, J = 7.4 Hz, 1H), 7.23 (q, J = 7.2, 6.5 Hz, 1H), 7.02 (td, J = 8.8, 2.8 Hz, 1H), 4.77-4.62 (m, 2H), 4.61 (d, J = 4.0 Hz, 1H), 4.42 (d, J = 14.2 Hz, 1H), 4.33-4.22 (m, 1H), 4.11 (dq, J = 11.6, 6.1, 5.7 Hz, 1H), 3.97 (d, J = 18.1 Hz, 1H), 3.61-3.55 (m, 2H), 2.88-2.76 (m, 1H), 2.61-2.49 (m, 1H), 2.41 (d, J = 7.8 Hz, 3H), 2.31 (ddd, J = 17.7, 13.7, 7.1 Hz, 2H), 2.24 (ddd, J = 16.1, 8.4, 3.1 Hz, 2H), 2.19-2.07 (m, 6H), 2.07-1.83 (m, 2H). |
| 271 | 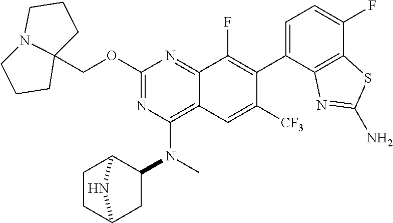 | LCMS m/z [M + 1]: 646.3 ¹HNMR (500 MHz, CD₃OD) δ 8.35 (s, 1H), 7.25-7.18 (m, 1H), 7.00 (ddd, J = 9.3, 8.4, 0.9 Hz, 1H), 4.74-4.64 (m, 2H), 4.54-4.46 (m, 1H), 4.32-4.27 (m, 1H), 3.72-3.63 (m, 3H), 3.49-3.45 (m, 3H), 3.36-3.32 (m, 1H), 2.66 (s, 2H), 2.32 (dd, J = 12.8, 6.9 Hz, 4H), 2.29-2.08 (m, 6H), 2.08-1.82 (m, 3H). |
| 272 | 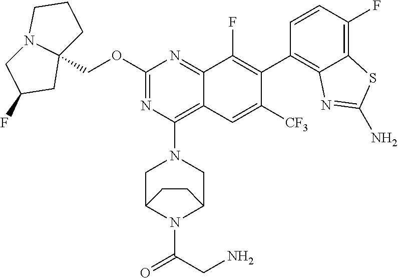 [SMILES: NCC(=O)N1C2CCC1CN(c1nc(OC[C@@]34CCCN3C[C@H](F)C4)nc3c(F)c(-c4ccc(F)c5sc(N)nc45)c(C(F)(F)F)cc13)C2] | LCMS m/z [M + 1]: 707.3 ¹HNMR (400 MHz, CD₃OD) δ 8.17 (s, 1H), 7.18-7.24 (m, 1H), 7.0 (t, J = 8.8 Hz, 1H), 5.56 (d, J = 51.6 Hz, 1H), 4.80-4.84 (m, 1H) 4.57-4.79(m, 4H), 4.41-4.46 (m, 1H), 4.03-4.11 (m, 2H), 3.81-3.99 (m, 4H), 3.69-3.79 (m, 1H), 3.42-3.51 (m, 1H), 2.53-2.76 (m, 2H) 2.28-2.48 (m, 3H), 2.02-2.20 (m, 2H), 1.82-2.00 (m, 3H). |
| 273 | 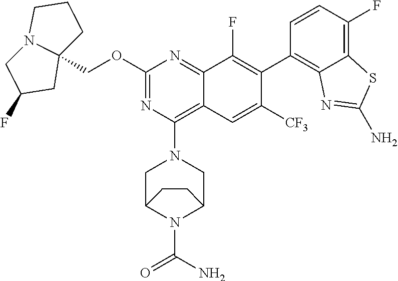 [SMILES: NC(=O)N1C2CCC1CN(c1nc(OC[C@@]34CCCN3C[C@H](F)C4)nc3c(F)c(-c4ccc(F)c5sc(N)nc45)c(C(F)(F)F)cc13)C2] | LCMS m/z [M + 1]: 693.2 ¹HNMR (400 MHz, CD₃OD) δ 8.21 (s, 1H), 7.20-7.25 (m, 1H), 7.01 (t, J = 8.8 Hz, 1H), 5.45 (d, J = 51.6 Hz, 1H), 4.52-4.74 (m, 4H), 4.44 (s, 2H), 3.77-4.07 (m, 5H), 3.42-3.50 (m, 1H), 2.54-2.7 (m, 2H), 2.30- 2.47 (m, 3H), 2.10-2.22 (m, 1H), 1.95-2.03 (m, 2H), 1.73-1.85 (m, 2H). |
| 274 | 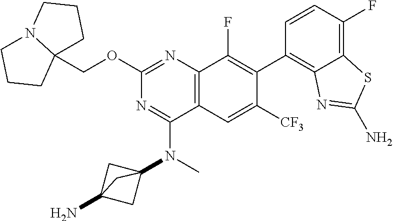 | LCMS m/z [M + 1]: 632.3 ¹H NMR (400 MHz, CD₃OD) δ 8.34 (s, 1H), 7.20 (dd, J = 8.0, 4.8 Hz, 1H), 6.98 (t, J = 8.8 Hz, 1H), 4.66 (s, 2H), 3.61-3.72 (m, 2H), 3.51 (s, 3H), 3.23-3.48 (m, 2H), 2.68 (s, 6H), 2.10-2.35 (m, 8H). |
| 276 | 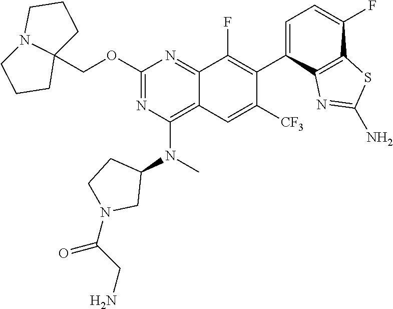 | LCMS m/z [M + 1]: 677.3 ¹HNMR (400 MHz, CD₃OD) δ 8.38 (s, 1H), 7.20-7.30 (m, 1H), 6.99 (t, J = 8.8 Hz, 1H), 5.23-5.51 (m, 1H), 4.67 (s, 2H), 3.76-4.2 (m, 4H), 3.55-3.76 (m, 4H), 3.39-3.55 (m, 3H), 3.18-3.25 (m, 2H), 1.91-2.62(m, 10H). |
| 277 | 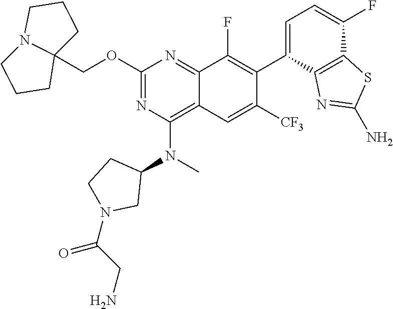 | LCMS m/z [M + 1]: 677.3 ¹HNMR (400 MHz, CD₃OD) δ 8.31-8.40 (m, 1H), 7.21 (dd, J = 6.0, 8.4 Hz, 1H), 6.99 (t, J = 8.4 Hz, 1H), 5.23-5.51 (m, 1H), 4.67 (s, 2H), 3.76-4.21 (m, 4H), 3.55-3.76 (m, 4H), 3.45-3.55 (m, 3H), 3.19-3.30 (m, 2H), 1.91- 2.62 (m, 10H). |
| 278 | 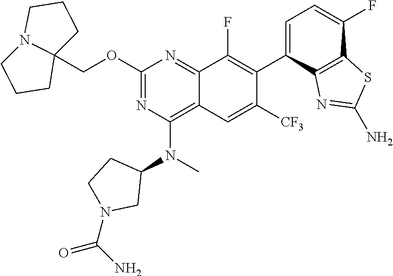 | LCMS m/z [M + 1]: 663.2 ¹HNMR (400 MHz, CD₃OD) δ 8.37 (s, 1H), 7.20-7.29 (m, 1H), 7.02 (t, J = 8.8 Hz, 1H), 5.25-5.37 (m, 1H), 4.61-4.73 (m, 2H), 3.89- 3.99 (m, 1H), 3.53-3.79 (m, 3H), 3.39-3.58 (m, 5H), 3.22-3.30 (m, 2H), 2.03-2.54 (m, 10H). |
| 279 | 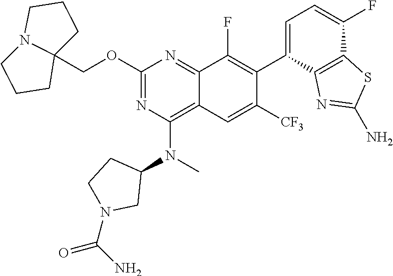 | LCMS m/z [M + 1]: 663.2 ¹HNMR (400 MHz, CD₃OD) δ 8.37 (s, 1H), 7.20-7.29 (m, 1H), 7.02 (t, J = 8.8 Hz, 1H), 5.25-5.37 (m, 1H), 4.67 (s, 2H), 3.89-3.99 (m, 1H), 3.53-3.79 (m, 3H), 3.39-3.58 (m, 5H), 3.22-3.30 (m, 2H), 2.03-2.54 (m, 10H). |
| 280 | 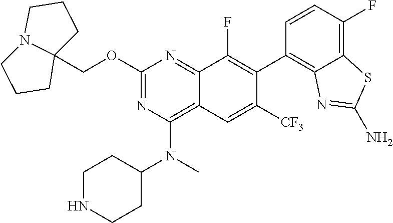 [SMILES: CN(c1nc(OCC23CCCN2CCC3)nc2c(F)c(-c3ccc(F)c4sc(N)nc34)c(C(F)(F)F)cc12)C1CCNCC1] | LCMS m/z [M + 1]: 634.2 ¹HNMR(400 MHz, CD₃OD) δ 8.36 (s, 1H), 7.18-7.24 (m,1H), 6.99 (t, J = 8.8 Hz, 1H), 4.92-5.04 (m, 1H), 4.67 (s, 2H), 3.58-3.73 (m, 4H), 3.47 (s, 3H), 3.20-3.31 (m, 4H), 2.05-2.35 (m, 12H). |

Additional compounds of the present disclosure are included in Table 8.

TABLE 8
Selected compounds of the present disclosure.

| Compound | | |
|---|---|---|
| No. | Structure | Analytical data |
| 119 | 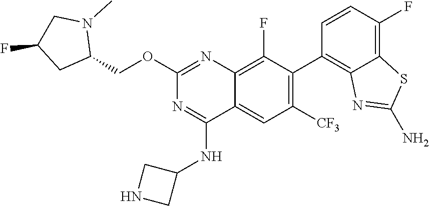 [SMILES: CN1C[C@H](F)C[C@H]1COc1nc(NC2CNC2)c2cc(C(F)(F)F)c(-c3ccc(F)c4sc(N)nc34)c(F)c2n1] | LCMS m/z [M + 1]: 584.3 ¹HNMR (400 MHz, CD3OD): δ 8.57 (s, 1H), 7.18-7.21 (m, 1H), 6.99 (td, J = 2.4, 9.2 Hz, 1H), 5.35 (dd, J = 2.8, 51.6 Hz, 1H), 5.19- 5.23 (m, 1H), 4.98-5.02 (m, 1H), 4.68-4.73 (m, 1H), 4.47-4.52 (m, 2H), 4.35-4.40 (m, 2H ), 4.19-4.31 (m, 1H), 3.95-4.16 (m, 1H), 3.58-3.75 (m, 1H), 3.17 (s, 3H), 2.67-2.78 (m, 1H), 2.25-2.49 (m, 1H). |
| 121 | 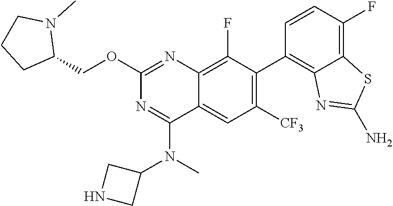 [SMILES: CN(c1nc(OC[C@@H]2CCCN2C)nc2c(F)c(-c3ccc(F)c4sc(N)nc34)c(C(F)(F)F)cc12)C1CNC1] | |
| 122 | 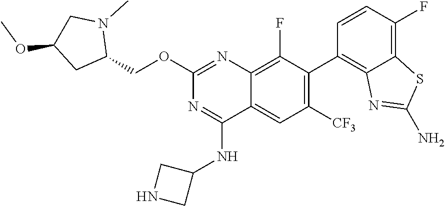 [SMILES: CO[C@@H]1C[C@@H](COc2nc(NC3CNC3)c3cc(C(F)(F)F)c(-c4ccc(F)c5sc(N)nc45)c(F)c3n2)N(C)C1] | LCMS m/z [M + 1]: 596.0 ¹HNMR (400 MHz, CD₃OD) δ 8.56 (s, 1H), 7.17-7.22 (m, 1H), 6.96-7.02 (m, 1H), 5.14- 5.31 (m, 1H), 4.91-4.97 (m, 2H), 4.61-4.68 (m, 1H), 4.33-4.56 (m, 4H), 4.07-4.16 (m, 2H), 3.80-3.84 (m, 1H), 3.39 (s, 3H), 3.12 (s, 3H), 2.45-2.54 (m, 1H), 2.13-2.19 (m, 1H). |
| 125 | 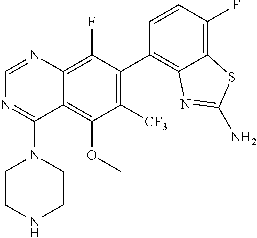 | LCMS m/z [M + 1]: 463.2 ¹HNMR(400 MHz, CD₃OD): 8.72 (s, 1H), 7.25-7.28 (m, 1H), 7.02-7.06 (m, 1H), 3.95- 4.10 (br, 4H), 3.88 (s, 3H), 3.45-3.48 (m, 4H). |
| 128 | 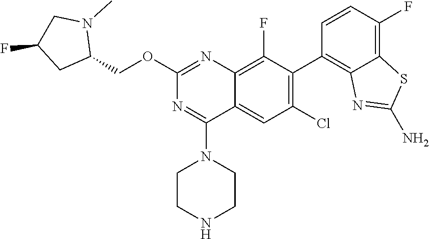 [SMILES: CN1C[C@H](F)C[C@H]1COc1nc(N2CCNCC2)c2cc(Cl)c(-c3ccc(F)c4sc(N)nc34)c(F)c2n1] | LCMS m/z [M + 1]: 564.3 ¹HNMR (400 MHz, CD₃OD) δ 8.00 (s, 1H), 7.20-7.26 (m, 1H), 7.02 (t, J = 8.8 Hz, 1H), 5.47 (d, J = 52.4 Hz, 1H), 4.95-5.03 (m, 1H), 4.70-4.77 (m, 1H), 4.21-4.31 (m, 1H), 3.95- 4.18 (m, 5H), 3.58-3.73 (m, 1H), 3.46-3.54 (m, 4H), 3.18 (s, 3H), 2.60-2.74 (m, 1H), 2.30-2.50 (m, 1H). |
| 129 | 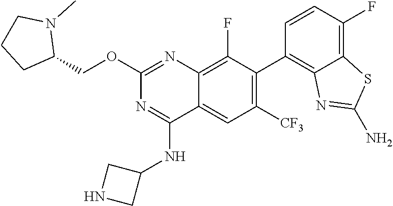 [SMILES: CN1CCC[C@H]1COc1nc(NC2CNC2)c2cc(C(F)(F)F)c(-c3ccc(F)c4sc(N)nc34)c(F)c2n1] | |
| 130 | 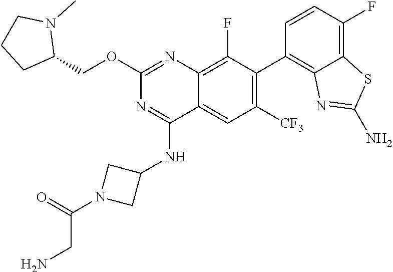 [SMILES: CN1CCC[C@H]1COc1nc(NC2CN(C(=O)CN)C2)c2cc(C(F)(F)F)c(-c3ccc(F)c4sc(N)nc34)c(F)c2n1] | |
| 131 | 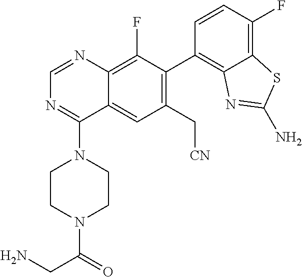 [SMILES: N#CCc1cc2c(N3CCN(C(=O)CN)CC3)ncnc2c(F)c1-c1ccc(F)c2sc(N)nc12] | |
| 132 | 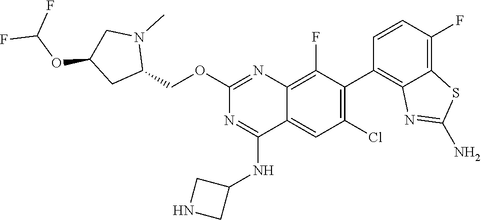 [SMILES: CN1C[C@H](OC(F)F)C[C@H]1COc1nc(NC2CNC2)c2cc(Cl)c(-c3ccc(F)c4sc(N)nc34)c(F)c2n1] | LCMS m/z [M + 1]: 598.3 ¹HNMR (400 MHz, CD₃OD) δ 8.23 (s, 1H), 7.21-7.25 (m, 1H), 7.02 (t, J = 8.8 Hz, 1H), 6.56 (t, J = 72 Hz, 1H), 5.06-5.17 (m, 2H), 4.65-4.70 (m, 1H), 4.46-4.51 (m, 2H), 4.34-4.38 (m, 2H), 4.16-4.21 (m, 1H), 4.01- 4.03 (m, 1H), 3.48-3.52 (m, 2H), 3.06-3.17 (m, 3H), 3.40-3.53 (m, 2H). |
| 133 | 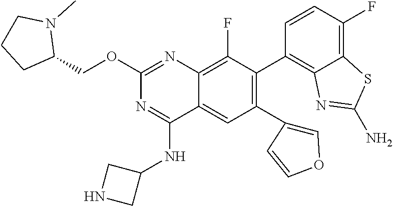 [SMILES: CN1CCC[C@H]1COc1nc(NC2CNC2)c2cc(-c3ccoc3)c(-c3ccc(F)c4sc(N)nc34)c(F)c2n1] | LCMS m/z [M + 1]: 564.2 ¹HNMR (400 MHz, CD₃OD) δ 8.19 (s, 1H), 7.33 (s, 1H), 6.88-7.21 (m, 3H), 6.24 (s, 1H), 5.18-5.32(m, 2H), 4.62-4.69 (m, 1H), 4.43- 4.58 (m, 2H), 4.31-4.44 (m, 2H), 3.60- 3.98 (m, 3H), 3.08 (s, 3H), 2.32-2.48 (m, 1H), 2.03-2.24 (m, 3H). |
| 134 | 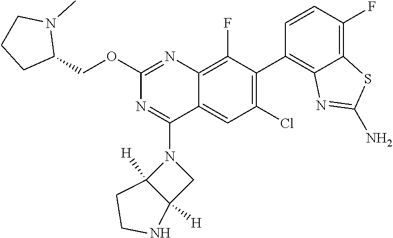 | LCMS m/z [M + 1]: 576.1 ¹HNMR (400 MHz, CD₃OD) δ 7.83 (s, 1H), 7.20-7.25 (m, 1H), 7.00-7.05 (m, 1H), 5.52-5.62 (m, 1H), 5.39-5.52 (m, 1H), 4.62-5.10 (m, 6H), 4.18-4.30 (m, 1H), 3.88- 4.15 (m, 1H), 3.79-3.88 (m, 2H), 3.58-3.70 (m, 1H), 3.17 (s, 3H), 2.62-2.77 (m, 2H), 2.21- 2.50 (m, 2H). |
| 135 | 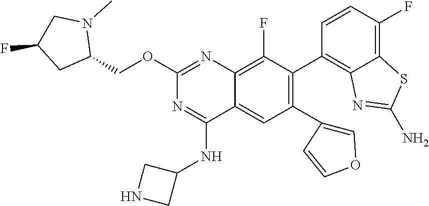 [SMILES: CN1C[C@H](F)C[C@H]1COc1nc(NC2CNC2)c2cc(-c3ccoc3)c(-c3ccc(F)c4sc(N)nc34)c(F)c2n1] | LCMS m/z [M + 1]: 582.1 ¹HNMR (400 MHz, CD₃OD): δ 8.21 (s, 1H), 7.35 (s, 1H), 7.05-7.11 (m, 2H), 6.97 (t, J = 8.8 Hz, 1H), 6.25 (s, 1H), 5.44 (d, J = 51.2 Hz, 1H), 5.23-5.30 (m, 1H), 5.05-5.15 (m, 1H), 4.73-4.82 (m,1H), 4.35-4.85 (m, 4H), 4.01-4.28 (m, 2H), 3.55-3.80 (m, 1H), 3.30 (s, 3H), 2.63-2.70 (m, 1H), 2.32-2.44 (m, 1H). |
| 136 | 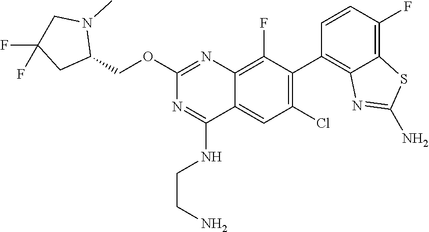 [SMILES: CN1CC(F)(F)C[C@H]1COc1nc(NCCN)c2cc(Cl)c(-c3ccc(F)c4sc(N)nc34)c(F)c2n1] | LCMS m/z [M + 1]: 556.1 ¹HNMR (700 MHz, CD₃OD) δ 8.69 (s, 1H), 8.18 (d, J = 1.5 Hz, 1H), 7.36 (dd, J = 8.5, 5.2 Hz, 1H), 7.11 (t, J = 8.8 Hz, 1H), 4.94- 4.91 (m, 1H), 3.88 (s, 2H), 3.84 (s, 1H), 2.86 (tt, J = 10.7, 4.4 Hz, 1H), 2.66 (s, 3H), 2.21- 2.14 (m, 3H), 2.04-1.95 (m, 2H), 1.33 (s, 1H). |
| 137 | 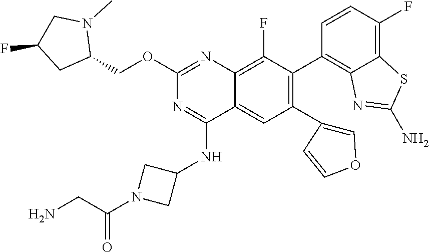 [SMILES: CN1C[C@H](F)C[C@H]1COc1nc(NC2CN(C(=O)CN)C2)c2cc(-c3ccoc3)c(-c3ccc(F)c4sc(N)nc34)c(F)c2n1] | LCMS m/z [M + 1]: 639.4 ¹HNMR (400 MHz, CD₃OD): δ 8.26 (s, 1H), 7.35 (s, 1H), 6.94-7.10 (m, 3H), 6.25 (s, 1H), 5.45 (d, J = 52.4 Hz, 1H), 5.05-5.19 (m, 2H), 4.69-4.85 (m, 2H), 4.54-4.58 (m, 1H), 4.25-4.36 (m, 3H), 4.04-4.10 (m, 1H), 3.70- 3.98 (m, 3H), 3.17 (s, 3H), 2.62-2.70 (m, 1H), 2.35-2.44 (m, 1H). |
| 138 | 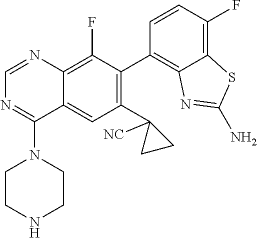 [SMILES: N#CC1(c2cc3c(N4CCNCC4)ncnc3c(F)c2-c2ccc(F)c3sc(N)nc23)CC1] | |
| 139 | 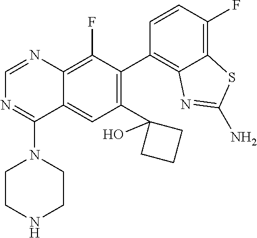 [SMILES: Nc1nc2c(-c3c(C4(O)CCC4)cc4c(N5CCNCC5)ncnc4c3F)ccc(F)c2s1] | LCMS m/z [M + 1]: 469.15 ¹HNMR (300 MHz, CD₃OD) δ 8.61 (s, 1H), 7.74 (s, 1H), 7.53-7.24 (m, 1H), 6.99 (t, J = 8.9 Hz, 1H), 3.94 (m, 4H), 3.06 (m, 4H), 2.50-2.36 (m, 1H), 2.35-2.19 (m, 1H), 2.21-1.96 (m, 2H), 1.88-1.70 (m, 1H), 1.71-1.55 (m, 1H). |
| 140 | 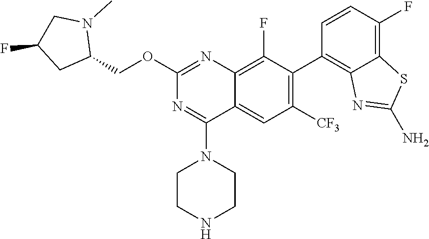 [SMILES: CN1C[C@H](F)C[C@H]1COc1nc(N2CCNCC2)c2cc(C(F)(F)F)c(-c3ccc(F)c4sc(N)nc34)c(F)c2n1] | LCMS m/z [M + 1]: 598.1 ¹H NMR (400 MHz, CD₃OD) δ 8.20 (s, 1H), 7.16-7.28 (m, 1H), 7.00 (t, J = 8.8 Hz, 1H), 5.47 (d, J = 52 Hz, 1H), 4.96-5.08 (m, 1H), 4.82-4.66 (m, 1H), 3.97-4.35 (m, 6H), 3.61- 3.75 (m, 1H), 3.42-3.55 (m, 4H), 3.13 (s, 3H), 2.29-2.86 (m, 2H). |
| 141 | 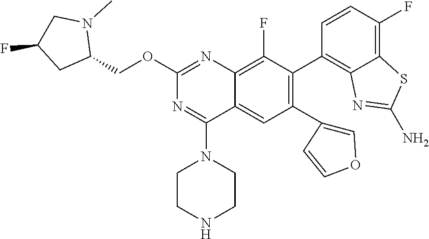 [SMILES: CN1C[C@H](F)C[C@H]1COc1nc(N2CCNCC2)c2cc(-c3ccoc3)c(-c3ccc(F)c4sc(N)nc34)c(F)c2n1] | LCMS m/z [M + 1]: 596.2 ¹HNMR (400 MHz, CD₃OD) δ 7.89 (s, 1H), 7.33 (s, 1H), 7.18 (s, 1H), 7.02-7.12 (m, 1H), 6.95 (t, J = 8.8 Hz, 1H), 6.15 (s, 1H), 5.47 (d, J = 53.0 Hz, 1H), 4.99-5.18 (m, 1H), 4.79- 4.66 (m, 1H), 3.95-4.31 (m, 6H), 3.57- 3.85 (m, 1H), 3.42-3.58 (m, 4H), 3.19 (s, 3H), 2.25-2.75 (m, 2H). |
| 142 | 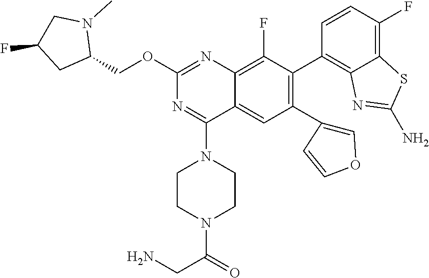 [SMILES: CN1C[C@H](F)C[C@H]1COc1nc(N2CCN(C(=O)CN)CC2)c2cc(-c3ccoc3)c(-c3ccc(F)c4sc(N)nc34)c(F)c2n1] | LCMS m/z [M + 1]: 653.1 ¹HNMR (400 MHz, CD₃OD) δ 8.47 (s, 1H), 7.86 (s, 1H), 7.30 (s, 1H), 7.11 (s, 1H), 7.04- 7.10 (m, 1H), 6.92 (t, J = 8.8 Hz, 1H), 6.14 (s, 1H), 5.11-5.32 (m, 2H), 4.59 (s, 1H), 4.56 (d, J = 4.2 Hz, 2H), 4.00-4.09 (m, 4H), 3.99 (s, 2H), 3.83-3.91 (m, 2H), 3.70-3.78 (m, 2H), 3.50-3.68 (m, 1H), 2.69-2.85 (m, 1H), 2.63 (s, 3H), 2.28-2.41 (m, 1H), 1.96-2.15 (m, 1H). |
| 143 | 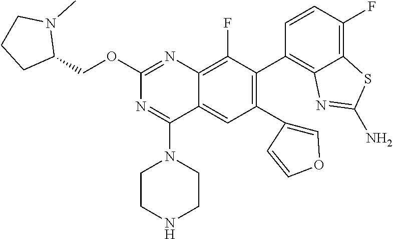 [SMILES: CN1CCC[C@H]1COc1nc(N2CCNCC2)c2cc(-c3ccoc3)c(-c3ccc(F)c4sc(N)nc34)c(F)c2n1] | LCMS m/z [M + 1]: 578.1 ¹HNMR (400 MHz, CD₃OD) δ 7.88 (s, 1H), 7.33 (s, 1H), 7.18 (s, 1H), 7.07-7.14 (m, 1H), 6.94-7.01 (m, 1H), 6.15 (s, 1H), 4.90- 5.00 (m, 2H), 4.66-4.75 (m, 1H), 4.18 (s, 4H), 3.86-3.96 (m, 1H), 3.68-3.80 (m, 1H), 3.45-3.57 (m, 4H), 3.10 (s, 3H), 2.36- 2.48 (m, 1H), 2.16-2.27 (m, 1H), 2.03- 2.16 (m, 2H). |
| 144 | 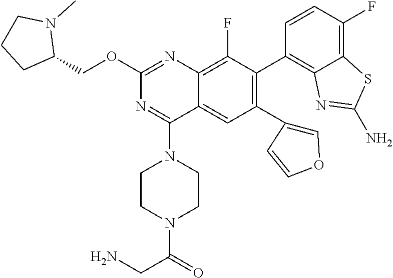 [SMILES: CN1CCC[C@H]1COc1nc(N2CCN(C(=O)CN)CC2)c2cc(-c3ccoc3)c(-c3ccc(F)c4sc(N)nc34)c(F)c2n1] | LCMS m/z [M + 1]: 635.1 ¹HNMR (400 MHz, CD₃OD) δ 7.96 (s, 1H), 7.33 (s, 1H), 7.19 (s, 1H), 7.11-7.17 (m, 1H), 6.98 (t, J = 8.8 Hz, 1H), 6.15 (s, 1H), 4.90- 5.08 (m, 2H), 4.70-4.80 (m, 1H), 4.14-4.29 (m, 4H), 4.04 (s, 2H), 3.92 (s, 3H), 3.77 (s, 3H), 3.10 (s, 3H), 2.36-2.50 (m, 1H), 2.18- 2.30 (m, 1H), 2.05-2.18 (m, 2H). |
| 145 | 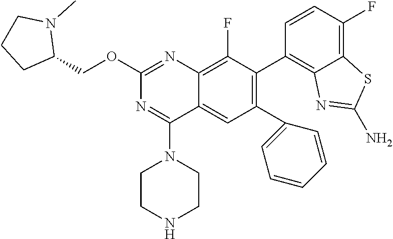 [SMILES: CN1CCC[C@H]1COc1nc(N2CCNCC2)c2cc(-c3ccccc3)c(-c3ccc(F)c4sc(N)nc34)c(F)c2n1] | LCMS m/z [M + 1]: 588.19 ¹HNMR (300 MHz, CD₃OD) δ 7.81 (s, 1H), 7.23-7.10 (m, 5H), 6.92 (dd, J = 8.6, 5.0 Hz, 1H), 6.78 (t, J = 9.0 Hz, 1H), 4.96 (d, J = 12.6 Hz, 1H), 4.73 (dd, J = 13.2, 6.4 Hz, 1H), 4.22-4.14 (m, 4H), 3.93 (s, 1H), 3.76 (s, 1H), 3.54-3.43 (m, 4H), 3.13 (s, 3H), 2.50- 2.04 (m, 5H). |
| 146 | 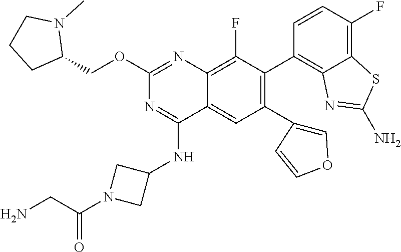 [SMILES: CN1CCC[C@H]1COc1nc(NC2CN(C(=O)CN)C2)c2cc(-c3ccoc3)c(-c3ccc(F)c4sc(N)nc34)c(F)c2n1] | LCMS m/z [M + 1]: 621.1 ¹HNMR (400 MHz, CD₃OD) δ 8.24 (s, 1H), 7.34 (s, 1H), 7.08-7.11 (m, 1H), 7.03 (s, 1H), 6.95 (t, J = 8.8 Hz, 1H), 6.25 (s, 1H), 5.08- 5.19 (s, 2H), 4.61-4.77 (m, 2H), 4.53-4.61 (m, 1H), 4.32-4.39 (m, 1H), 4.22-4.30 (m, 1H ), 3.68-3.92 (m, 4H), 3.19-3.28 (m, 1H), 3.09 (s, 3H), 2.02-2.49 (m, 4H). |
| 147 | 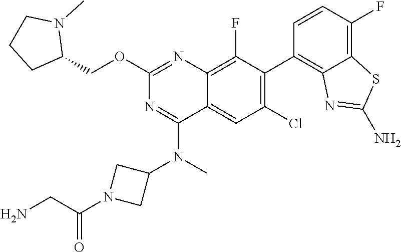 [SMILES: CN(c1nc(OC[C@@H]2CCCN2C)nc2c(F)c(-c3ccc(F)c4sc(N)nc34)c(Cl)cc12)C1CN(C(=O)CN)C1] | |
| 148 | 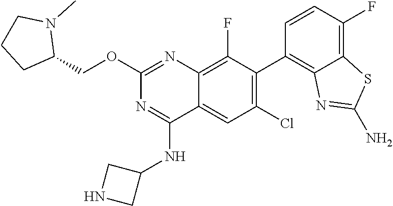 | LCMS m/z [M + 1]: 516.0 ¹HNMR (400 MHz, CD₃OD) δ 8.23 (s, 1H), 7.10-7.18 (m, 1H), 6.98-7.08 (m, 1H), 5.15- 5.25 (m, 1H), 4.60-4.70 (m, 1H), 4.30-4.55 (m, 4H), 3.70-3.95 (m, 2H), 3.20-3.25 (m, 1H), 3.09 (s, 3H), 2.35-2.45 (m, 1H), 2.00- 2.30 (m, 3H). |
| 149 | 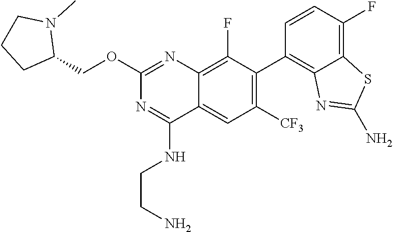 [SMILES: CN1CCC[C@H]1COc1nc(NCCN)c2cc(C(F)(F)F)c(-c3ccc(F)c4sc(N)nc34)c(F)c2n1] | LCMS m/z [M + 1]: 554.07 ¹HNMR (300 MHz, CD₃OD) δ 8.46 (s, 1H), 7.19 (t, J = 7.0 Hz, 1H), 6.98 (t, J = 8.8 Hz, 1H), 4.98-4.86 (m, 1H), 4.79-4.57 (m, 1H), 4.13-3.61 (m, 4H), 3.09 (s, 3H), 2.50- 2.34 (m, 1H), 2.22-2.02 (m, 3H), 1.49- 1.03 (m, 3H). |
| 150 | 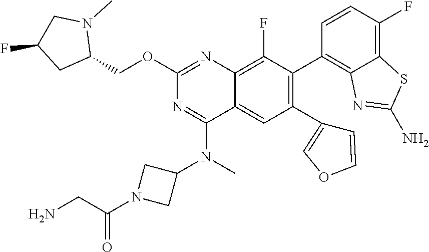 [SMILES: CN(c1nc(OC[C@@H]2C[C@@H](F)CN2C)nc2c(F)c(-c3ccc(F)c4sc(N)nc34)c(-c3ccoc3)cc12)C1CN(C(=O)CN)C1] | |
| 152 | 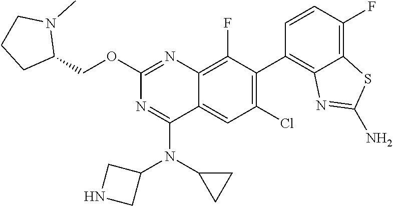 [SMILES: CN1CCC[C@H]1COc1nc(N(C2CC2)C2CNC2)c2cc(Cl)c(-c3ccc(F)c4sc(N)nc34)c(F)c2n1] | LCMS m/z [M + 1]: 572.2 |
| 154 | 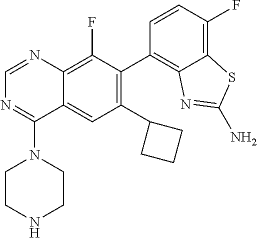 [SMILES: Nc1nc2c(-c3c(C4CCC4)cc4c(N5CCNCC5)ncnc4c3F)ccc(F)c2s1] | LCMS m/z [M + 1]: 453.18 ¹HNMR (300 MHz, CD₃OD) δ 8.84 (s, 1H), 7.88 (s, 1H), 7.28 (dd, J = 8.5, 5.5 Hz, 1H), 7.12 (t, J = 8.8 Hz, 1H), 4.59 (m, 4H), 3.64 (m, 1H), 3.57 (m, 4H), 2.28-1.98 (m, 3H), 1.88-1.68 (m, 3H). |
| 155 | 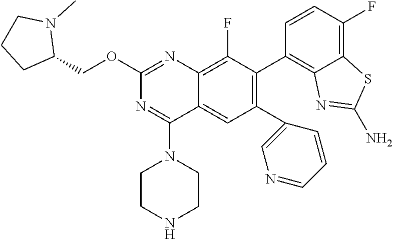 [SMILES: CN1CCC[C@H]1COc1nc(N2CCNCC2)c2cc(-c3cccnc3)c(-c3ccc(F)c4sc(N)nc34)c(F)c2n1] | LCMS m/z [M + 1]: 589.28 ¹HNMR (300 MHz, CD₃OD) δ 8.70 (s, 2H), 8.15 (s, 1H), 7.99 (s, 1H), 7.84-7.61 (m, 4H), 7.27 (s, 1H), 6.99-6.89 (m, 1H), 4.99 (s, 1H), 4.75 (dd, J = 12.5, 6.5 Hz, 1H), 4.21 (t, J = 5.5 Hz, 4H), 3.94 (s, 1H), 3.78 (s, 1H), 3.57- 3.45 (m, 4H), 3.13 (s, 3H), 2.50-2.38 (m, 1H), 2.29-2.07 (m, 3H). |
| 156 | 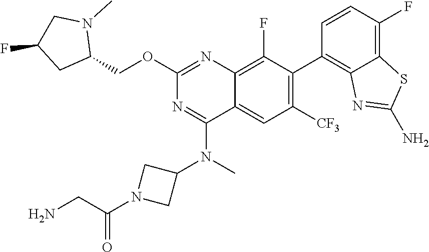 [SMILES: CN(c1nc(OC[C@@H]2C[C@@H](F)CN2C)nc2c(F)c(-c3ccc(F)c4sc(N)nc34)c(C(F)(F)F)cc12)C1CN(C(=O)CN)C1] | LCMS m/z [M+1]: 655.1 |
| 160 | 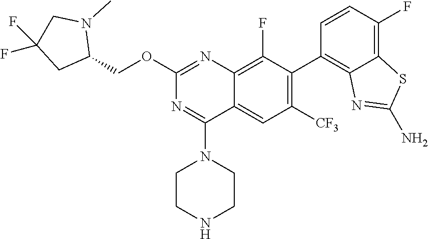 [SMILES: CN1CC(F)(F)C[C@H]1COc1nc(N2CCNCC2)c2cc(C(F)(F)F)c(-c3ccc(F)c4sc(N)nc34)c(F)c2n1] | LCMS m/z [M + 1]: 616.0 ¹HNMR (400 MHz, CD₃OD) δ 8.18 (s, 1H), 7.17-7.22 (m, 1H), 6.99 (t, J = 8.65 Hz, 1H), 4.97-5.00 (m, 1H), 4.74-4.77 (m, 1H), 4.16- 4.21 (m, 5H), 4.10-4.13 (m, 1H), 3.70-3.80 (m, 1H), 3.47 (s, 4H), 3.11 (s, 3H), 2.88-2.96 (m, 1H), 2.67-2.73 (m, 1H). |
| 166 | 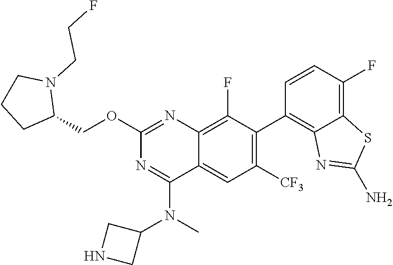 [SMILES: CN(c1nc(OC[C@@H]2CCCN2CCF)nc2c(F)c(-c3ccc(F)c4sc(N)nc34)c(C(F)(F)F)cc12)C1CNC1] | LCMS m/z [M + 1]: 612.1 ¹HNMR (400 MHz, CD₃OD) δ 8.52 (s, 1H), 7.12-7.24 (m, 1H), 7.01 (t, J = 8 Hz, 1H), 5.01-5.27 (m, 4H), 4.79-4.88 (m, 2H), 4.12- 4.27 (m, 1H), 3.81-3.96 (m, 5H), 3.60-3.76 (m, 4H), 3.35-3.48 (m, 1H), 2.44-2.46 (m, 1H), 2.18-2.20 (m, 3H). |
| 168 | 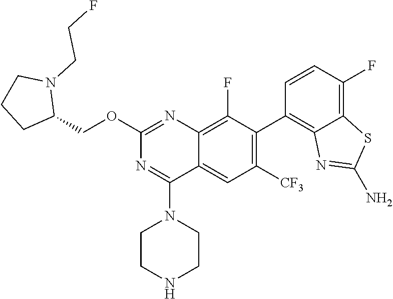 [SMILES: Nc1nc2c(-c3c(C(F)(F)F)cc4c(N5CCNCC5)nc(OC[C@@H]5CCCN5CCF)nc4c3F)ccc(F)c2s1] | LCMS m/z [M + 1]: 612.2 ¹HNMR (400 MHz, CD₃OD) δ 8.20 (s, 1H), 7.17-7.26 (m, 1H), 7.02 (t, J = 8.8 Hz, 1H), 4.69-4.84 (m, 2H), 4.17-4.29 (m, 4H), 3.96- 4.17 (m, 2H), 3.72-3.92 (m, 1H), 3.42-3.71 (m, 6H), 3.35-3.42 (m, 2H), 2.05-2.50 (m, 4H). |
| 170 | 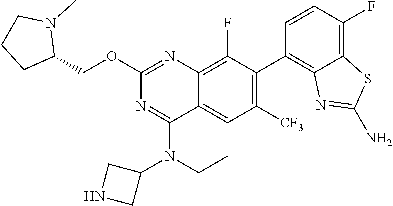 [SMILES: CCN(c1nc(OC[C@@H]2CCCN2C)nc2c(F)c(-c3ccc(F)c4sc(N)nc34)c(C(F)(F)F)cc12)C1CNC1] | LCMS m/z [M + 1]: 594.1 ¹HNMR(400 MHz, CD₃OD): δ 8.41 (s, 1H), 7.15-7.25 (m, 1H), 7.01 (t, J = 7.2 Hz, 1H), 5.05-5.35 (m, 2H), 4.95-5.00 (m, 1H), 4.35- 4.50 (m, 1H), 4.15-4.30 (m, 1H), 3.90-4.10 (m, 1H), 3.55-3.85 (m, 3H), 3.22-3.25 (m, 1H), 3.10 (s, 3H), 2.40-2.50 (m, 1H), 2.10- 2.30 (m, 3H), 1.60 (t, J = 7.2 Hz, 3H). |
| 172 | 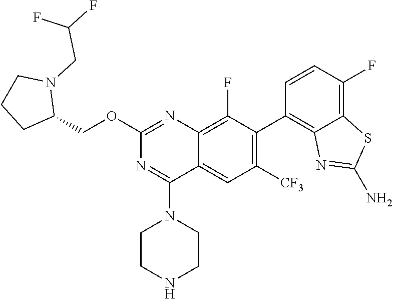 [SMILES: Nc1nc2c(-c3c(C(F)(F)F)cc4c(N5CCNCC5)nc(OC[C@@H]5CCCN5CC(F)F)nc4c3F)ccc(F)c2s1] | LCMS m/z [M + 1]: 630.0 ¹HNMR (400 MHz, CD₃OD) δ 8.20 (s, 1H), 7.20-7.24 (m, 1H), 7.02 (t, J = 8.8 Hz, 1H), 6.42 (t, J = 53.3 Hz, 1H), 4.71-4.78 (m, 1H), 4.17-4.27 (m, 6H), 3.64-3.97 (m, 2H), 3.38-3.50 (m, 5H), 2.03-2.44 (m, 4H). |
| 174 | 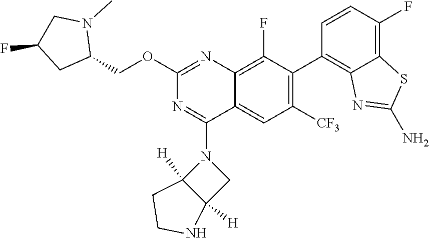 [SMILES: CN1C[C@H](F)C[C@H]1COc1nc(N2C[C@H]3NCC[C@H]32)c2cc(C(F)(F)F)c(-c3ccc(F)c4sc(N)nc34)c(F)c2n1] | LCMS m/z [M + 1]: 610.0 |
| 175 | 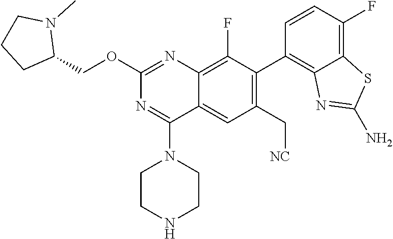 [SMILES: CN1CCC[C@H]1COc1nc(N2CCNCC2)c2cc(CC#N)c(-c3ccc(F)c4sc(N)nc34)c(F)c2n1] | LCMS m/z [M + 1]: 551.23 ¹HNMR (300 MHz, CD₃OD) δ 7.98 (s, 1H), 7.27 (dd, J = 8.5, 5.4 Hz, 1H), 7.04 (t, J = 8.8 Hz, 1H), 4.69 (dd, J = 13.0, 6.4 Hz, 1H), 4.15 (t, J = 4.8 Hz, 4H), 3.94 - 3.73 (m, 4H), 3.53 (t, J = 5.4 Hz, 4H), 3.10 (s, 3H), 2.45- 2.37 (m, 1H), 2.27-2.06 (m, 4H). |
| 176 | 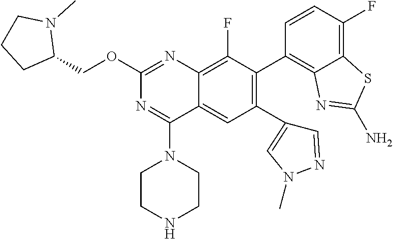 [SMILES: CN1CCC[C@H]1COc1nc(N2CCNCC2)c2cc(-c3cnn(C)c3)c(-c3ccc(F)c4sc(N)nc34)c(F)c2n1] | LCMS m/z [M + 1]: 592.40 ¹HNMR (300 MHz, CD₃OD) δ 7.93 (d, J = 1.4 Hz, 1H), 7.36 (s, 1H), 7.21 (ddd, J = 8.4, 5.3, 1.0 Hz, 1H), 7.12 (s, 1H), 7.08 (td, J = 9.2, 8.8, 2.0 Hz, 1H), 4.99 (dd, J = 12.8 Hz, 2.6 Hz, 1H), 4.76 (dt, J = 12.9, 6.5 Hz, 1H), 4.32-4.24 (m, 4H), 3.94 (s, 1H), 3.77 (s, 4H), 3.58-3.48 (m, 4H), 3.29-3.20 (br s, 1H), 3.13 (s, 3H), 2.44 (q, J = 9.3 Hz, 1H), 2.30-2.05 (m, 3H). |
| 177 | 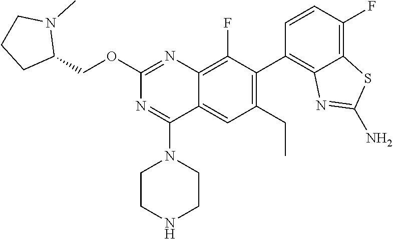 [SMILES: CCc1cc2c(N3CCNCC3)nc(OC[C@@H]3CCCN3C)nc2c(F)c1-c1ccc(F)c2sc(N)nc12] | LCMS m/z [M + 1]: 540.28 ¹HNMR (300 MHz, CD₃OD) δ 7.79 (s, 1H), 7.27 (m, 1H), 7.13 (m, 1H), 4.94 (m, 1H), 4.77 (m, 1H), 4.27 (m, 4H), 3.93 (m, 1H), 3.77 (m, 1H), 3.54 (m, 4H), 3.30-3.17 (m, 1H), 3.11 (s, 3H), 2.61 (dt, J = 14.2, 7.5 Hz, 2H), 2.42 (m, 1H), 2.08 (m, 3H), 1.09 (t, J = 7.4 Hz, 3H). |
| 178 | 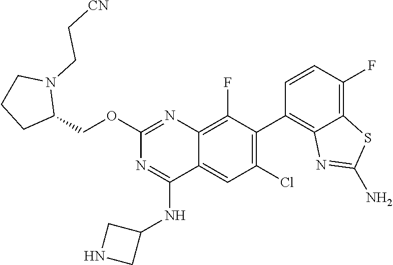 [SMILES: N#CCCN1CCC[C@H]1COc1nc(NC2CNC2)c2cc(Cl)c(-c3ccc(F)c4sc(N)nc34)c(F)c2n1] | LCMS m/z [M + 1]: 571.1 ¹HNMR (500 MHz, CD₃OD) δ 8.25 (q, J = 2.6, 2.1 Hz, 1H), 7.28 (ddd, J = 8.7, 5.3, 3.6 Hz, 1H), 7.07 (tt, J = 7.2, 3.6 Hz, 1H), 5.20 (dqt, J = 7.6, 5.4, 2.4 Hz, 1H), 4.86 (d, J = 5.9 Hz, 3H), 4.66 (ddd, J = 13.0, 7.5, 4.0 Hz, 1H), 4.51 (dddd, J = 10.5, 8.1, 4.7, 2.5 Hz, 2H), 4.36 (tt, J = 7.0, 3.4 Hz, 2H), 4.15- 4.06 (m, 1H), 3.98 (ddt, J = 14.0, 6.8, 3.5 Hz, 1H), 3.81 (p, J = 5.8 Hz, 1H), 3.60 (dtd, J = 12.8, 7.1, 3.0 Hz, 1H), 3.37 (dtd, J = 11.2, 7.6, 3.2 Hz, 1H), 3.18-3.04 (m, 2H), 2.66 (s, 1H), 2.46-2.35 (m, 1H), 2.28-1.99 (m, 3H). |
| 180 | 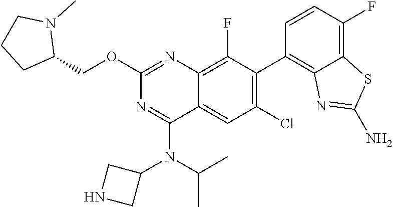 [SMILES: CC(C)N(c1nc(OC[C@@H]2CCCN2C)nc2c(F)c(-c3ccc(F)c4sc(N)nc34)c(Cl)cc12)C1CNC1] | LCMS m/z [M + 1]: 574.0 ¹HNMR (400 MHz, CD₃OD) δ 8.21-8.22 (m, 1H), 7.21-7.26 (m, 1H), 7.03 (t, J = 8.0 Hz, 1H), 5.10-5.28 (m, 4H), 4.74-4.78 (m, 2H), 3.56-3.96 (m, 4H), 3.11-3.27 (m, 4H), 2.14- 2.44 (m, 4H), 1.67-1.76 (m, 6H). |
| 183 | 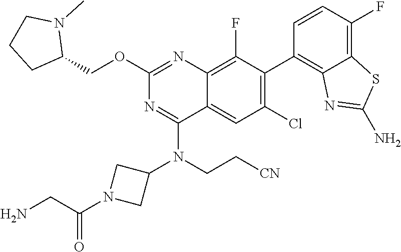 [SMILES: CN1CCC[C@H]1COc1nc(N(CCC#N)C2CN(C(=O)CN)C2)c2cc(Cl)c(-c3ccc(F)c4sc(N)nc34)c(F)c2n1] | LCMS m/z [M + 1]: 642.2 ¹HNMR (400 MHz, CD₃OD) δ 7.93 (s, 1H), 7.23 (t, J = 7.6 Hz, 1H), 7.02 (t, J = 9.2 Hz, 1H), 5.16 (t, J = 6.8 Hz, 1H), 4.43-4.66 (m, 3H), 4.18-4.24 (m, 3H), 3.85-3.92 (m, 1H), 3.71-3.78 (m, 3H), 3.22-3.27 (m, 2H), 3.09 (s, 3H), 2.86 (t, J = 6.4 Hz, 2H), 2.35-2.45 (m, 1H), 2.01-2.20 (m, 3H). |
| 191 | 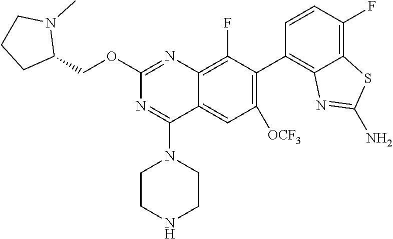 [SMILES: CN1CCC[C@H]1COc1nc(N2CCNCC2)c2cc(OC(F)(F)F)c(-c3ccc(F)c4sc(N)nc34)c(F)c2n1] | LCMS m/z [M + 1]: 596.26 ¹HNMR (300 MHz, CD₃OD) δ 7.79 (s, 1H), 7.42-7.24 (m, 1H), 7.15-7.00 (m, 1H), 5.01-4.92 (m, 1H), 4.78-4.60 (m, 1H), 4.19-3.99 (m, 4H), 3.96-3.83 (m, 1H), 3.82-3.68 (m, 1H), 3.56 3.39 (m, 4H), 3.26-3.17 (m, 1H), 3.09 (s, 3H), 2.46-2.33 (m, 1H), 2.27-2.00 (m, 3H). |
| 193 | 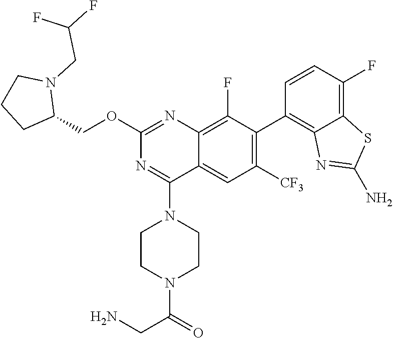 [SMILES: NCC(=O)N1CCN(c2nc(OC[C@@H]3CCCN3CC(F)F)nc3c(F)c(-c4ccc(F)c5sc(N)nc45)c(C(F)(F)F)cc23)CC1] | LCMS m/z [M + 1]: 687.2 ¹HNMR (400 MHz, CD₃OD) δ 8.23 (s, 1H), 7.17-7.25 (m, 1H), 7.00 (t, J = 8.8 Hz, 1H), 6.39 (t, J = 53.5 Hz, 1H), 4.67-4.77 (m, 1H), 4.02-4.19 (m, 8H), 4.03 (s, 3H), 3.69- 3.96 (m, 6H), 3.35-3.54 (m, 1H), 1.98- 2.48 (m, 4H). |
| 196 | 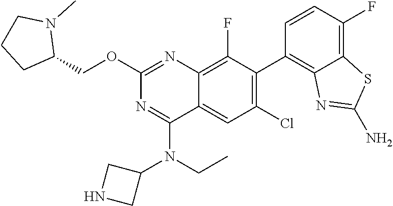 [SMILES: CCN(c1nc(OC[C@@H]2CCCN2C)nc2c(F)c(-c3ccc(F)c4sc(N)nc34)c(Cl)cc12)C1CNC1] | LCMS m/z [M + 1]: 560.0 ¹HNMR (400 MHz, CD₃OD) δ 8.26 (s, 1H), 7.20-7.27 (m, 1H), 7.03 (t, J = 8.8 Hz, 1H), 5.04-5.11 (m, 2H), 4.79-4.83 (m, 3H), 4.42- 4.48 (m, 1H), 4.10-4.16 (m, 2H), 3.58-3.75 (m, 3H), 3.20-3.3 (m, 1H), 3.11 (s, 3H), 2.39-2.52 (m, 1H), 2.14-2.21 (m, 3H), 1.57 (t, J = 6.8 Hz, 3H) |
| 197 | 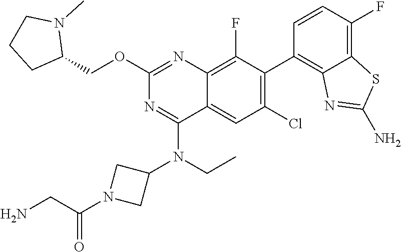 [SMILES: CCN(c1nc(OC[C@@H]2CCCN2C)nc2c(F)c(-c3ccc(F)c4sc(N)nc34)c(Cl)cc12)C1CN(C(=O)CN)C1] | LCMS m/z [M + 1]: 617.2 ¹HNMR (400 MHz, CD₃OD) δ 8.31 (s, 1H), 7.21-7.22 (m, 1H), 7.02 (t, J = 8.8 Hz, 1H), 5.14-5.29 (m, 1H), 4.62-4.74 (m, 2H), 4.17- 4.47 (m, 3H), 4.03 (s, 2H), 3.88-3.94 (m, 2H), 3.72-3.87 (m, 1H), 3.48-3.49 (m, 2H), 3.20-3.31 (m, 1H), 3.09 (s, 3H), 2.41-2.43 (m, 1H), 2.12-2.21 (m, 3H), 1.25-1.35 (m, 3H). |
| 200 | 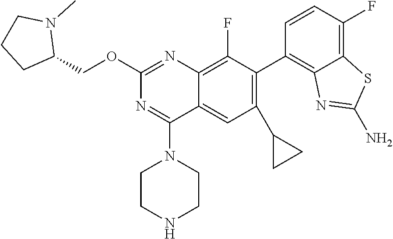 [SMILES: CN1CCC[C@H]1COc1nc(N2CCNCC2)c2cc(C3CC3)c(-c3ccc(F)c4sc(N)nc34)c(F)c2n1] | LCMS m/z [M + 1]: 574.13 ¹HNMR (300 MHz, CD₃OD) δ 7.44 (s, 1H), 7.32 (m, 1H), 7.13 (m, 1H), 4.95 (m, 1H), 4.73 (m, 1H), 4.33-4.10 (m, 4H), 3.92 (m, 1H), 3.75 (m, 1H), 3.51 (m, 4H), 3.11 (s, 3H), 2.44 (m, 1H), 2.06 (m, 4H), 1.74 (m, 1H), 1.02-0.49 (m, 4H). |
| 203 | 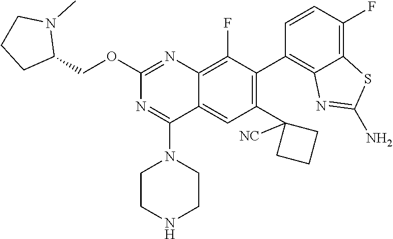 [SMILES: CN1CCC[C@H]1COc1nc(N2CCNCC2)c2cc(C3(C#N)CCC3)c(-c3ccc(F)c4sc(N)nc34)c(F)c2n1] | LCMS m/z [M + 1]: 590.79 ¹HNMR (300 MHz, CD₃OD) δ 7.67 (s, 1H), 7.32 (dd, J = 8.4, 5.5 Hz, 1H), 7.03 (t, J = 8.8 Hz, 1H), 4.77-4.53 (m, 1H), 4.15 (d, J = 5.4 Hz, 5H), 3.97-3.85 (m, 1H), 3.84- 3.73 (m, 1H), 3.50 (d, J = 5.4 Hz, 5H), 3.10 (d, J = 6.6 Hz, 3H), 2.89-2.66 (m, 1H), 2.45-2.34 (m, 1H), 2.26-2.09 (m, 5H), 1.86-1.76 (m, 1H), 1.68-1.57 (m, 1H). |
| 204 | 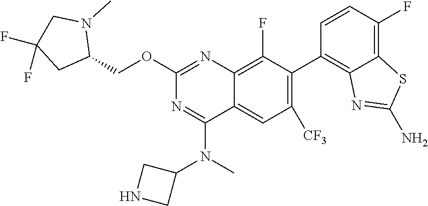 [SMILES: CN(c1nc(OC[C@@H]2CC(F)(F)CN2C)nc2c(F)c(-c3ccc(F)c4sc(N)nc34)c(C(F)(F)F)cc12)C1CNC1] | LCMS m/z [M + 1]: 616.1 ¹HNMR (400 MHz, CD₃OD): δ 8.52 (s, 1H), 7.23-7.19 (m, 1H), 7.01 (t, J = 8.8 Hz, 1H), 4.95-4.99 (m, 1H), 4.76-4.81 (m, 4H), 3.92 (s, 3H), 3.57-3.74 (m, 4H), 3.10-3.25 (m, 1H), 2.71-2.87 (m, 4H), 2.49-2.59 (m, 1H). |
| 205 | 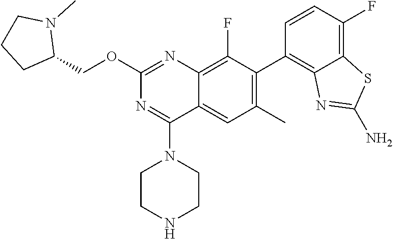 [SMILES: Cc1cc2c(N3CCNCC3)nc(OC[C@@H]3CCCN3C)nc2c(F)c1-c1ccc(F)c2sc(N)nc12] | LCMS m/z [M + Na]: 548.17 ¹HNMR (300 MHz, CD₃OD) δ 7.94 (d, J = 8.3 Hz, 0.5H), 7.81 (s, 0.5H), 7.60 (d, J = 8.6 Hz, 0.44H), 7.48-7.42 (m, 0.48H), 7.26 (d, J = 5.7 Hz, 0.6H), 7.10 (t, J = 8.9 Hz, 1H), 5.00 (s, 1H), 4.76 (dd, J = 12.8, 6.8 Hz, 1H), 4.29-4.20 (s, 4H), 3.58-3.48 (s, 4H), 3.77 (s, 1H), 3.53 (s, 1H), 3.29-3.23 (m, 4H), 3.12 (d, J = 5.1 Hz, 3H), 2.43 (s, 1H), 2.20 (m, 5H). |
| 208 | 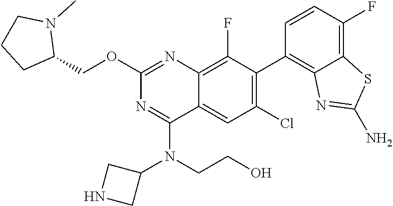 [SMILES: CN1CCC[C@H]1COc1nc(N(CCO)C2CNC2)c2cc(Cl)c(-c3ccc(F)c4sc(N)nc34)c(F)c2n1] | LCMS m/z [M + 1]: 576.2 ¹HNMR (400 MHz, CD₃OD) δ 8.28 (s, 1H), 7.22-7.30 (m, 1H), 7.00-7.12 (m, 1H), 4.96- 5.10 (m, 3H), 4.67-4.79 (m, 1H), 4.43-4.58 (m, 5H), 3.88-3.99 (m, 1H), 3.72-3.82 (m, 1H), 3.60-3.70 (m, 2H), 3.21-3.29 (m, 1H), 3.05-3.16 (m, 3H), 2.37-2.50 (m, 1H), 2.05- 2.27 (m, 3H). |
| 217 | 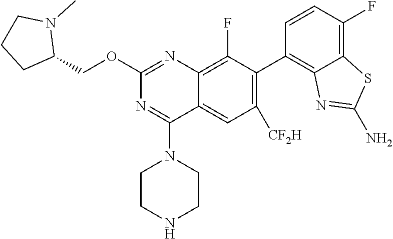 [SMILES: CN1CCC[C@H]1COc1nc(N2CCNCC2)c2cc(C(F)F)c(-c3ccc(F)c4sc(N)nc34)c(F)c2n1] | LCMS m/z [M + 1]: 562.19 ¹HNMR (300 MHz, CD₃OD) δ 8.15 (s, 1H), 7.36 (m, 1H), 7.15 (t, J = 8.7 Hz, 1H), 6.67 (t, J = 54.6 Hz, 1H), 4.95-4.88 (m, 1H), 4.75 (m, 1H), 4.23 (m, 4H), 4.19-4.09 (m, 1H), 3.94 (m, 1H), 3.77 (m, 1H), 3.54 (m, 4H), 3.12 (s, 3H), 2.45 (m, 1H), 2.19 (m, 3H). |
| 242 | 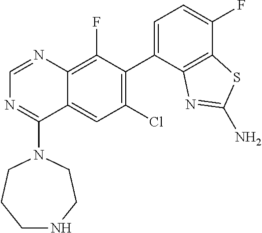 [SMILES: Nc1nc2c(-c3c(Cl)cc4c(N5CCCNCC5)ncnc4c3F)ccc(F)c2s1] | LCMS m/z [M + 1]: 447.0 ¹HNMR (500 MHz, CD₃OD) δ 8.76 (s, 1H), 8.25 (d, J = 1.7 Hz, 1H), 7.29 (dd, J = 8.4, 5.3 Hz, 1H), 7.05 (dd, J = 9.1, 8.5 Hz, 1H), 4.51-4.44 (m, 2H), 4.44-4.32 (m, 2H), 3.70-3.63 (m, 2H), 3.42-3.36 (m, 2H), 2.49-2.40 (m, 2H). |
| 243 | 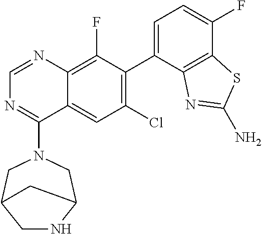 [SMILES: Nc1nc2c(-c3c(Cl)cc4c(N5CC6CNC(C6)C5)ncnc4c3F)ccc(F)c2s1] | LCMS m/z [M + 1]: 459.0 ¹HNMR (500 MHz, CD₃OD) δ 8.77 (d, J = 2.3 Hz, 1H), 8.17 (d, J = 1.7 Hz, 1H), 7.33- 7.21 (m, 1H), 7.10-7.02 (m, 1H), 5.29- 5.14 (m, 1H), 4.81-4.73 (m, 1H), 4.32- 4.24 (m, 1H), 4.16 (d, J = 14.0 Hz, 1H), 3.74 (dd, J = 25.5, 13.2 Hz, 1H), 3.48-3.35 (m, 2H), 2.97 (s, 1H), 2.42-2.32 (m, 1H), 2.23- 2.11 (m, 1H). |
| 244 | 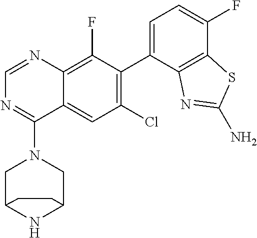 [SMILES: Nc1nc2c(-c3c(Cl)cc4c(N5CC6CCC(C5)N6)ncnc4c3F)ccc(F)c2s1] | LCMS m/z [M + 1]: 459.0 ¹HNMR (500 MHz, CD₃OD) δ 8.80 (s, 1H), 8.13 (s, 1H), 7.31 (dd, J = 8.5, 5.3 Hz, 1H), 7.08 (t, J = 8.8 Hz, 1H), 4.99-4.92 (m, 2H), 4.34-4.24 (m, 2H), 4.06 (dd, J = 14.1, 8.5 Hz, 2H), 2.14 (dd, J = 8.9, 4.1 Hz, 2H), 2.07 (d, J = 10.8 Hz, 2H). |
| 251 | 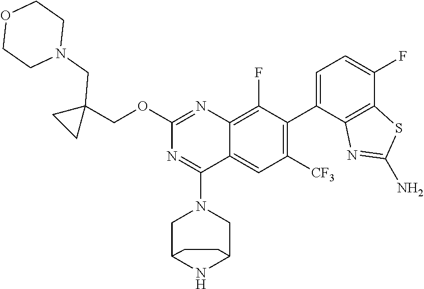 [SMILES: Nc1nc2c(-c3c(C(F)(F)F)cc4c(N5CC6CCC(C5)N6)nc(OCC5(CN6CCOCC6)CC5)nc4c3F)ccc(F)c2s1] | LCMS m/z [M + 1]: 662.3 ¹HNMR (400 MHz, CD₃OD) δ 8.14 (s, 1H), 7.17-7.22 (m, 1H), 6.99 (t, J = 8.4 Hz, 1H), 4.63-4.75 (m, 2H), 4.48 ( dd, J = 12 Hz, 49.2 Hz, 2H), 4.25 (s, 2H), 4.00-4.12 (m ,2H), 3.70-3.96 (m, 6H), 3.32-3.41 (m, 2H), 3.12- 3.26 (m,2H), 2.07-2.19 (m, 4H), 0.97-1.02 (m, 2H), 0.85-0.92 (m, 2H). |
| 252 | 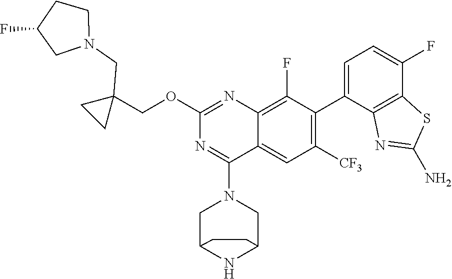 [SMILES: Nc1nc2c(-c3c(C(F)(F)F)cc4c(N5CC6CCC(C5)N6)nc(OCC5(CN6CC[C@@H](F)C6)CC5)nc4c3F)ccc(F)c2s1] | LCMS m/z [M + 1]: 664.3 ¹HNMR (400 MHz, CD₃OD) δ 8.15 (s, 1H), 7.17-7.22 (m, 1H), 7.0 (t, J = 8.8 Hz , 1H), 5.45 (d, J = 54.4 Hz, 1H), 4.72 (d, J = 14 Hz, 2H), 4.31-4.65 (m, 2H), 4.24 (s, 2H), 3.93 (d, J = 13.2 Hz, 3H), 3.15-3.67 (m, 5H), 2.25-2.45 (m, 2H) 2.05-2.19 (m, 4H), 0.85-1.01 (m, 4H). |
| 253 | 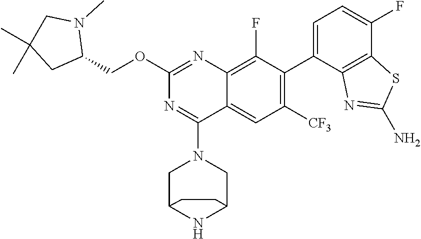 [SMILES: CN1CC(C)(C)C[C@H]1COc1nc(N2CC3CCC(C2)N3)c2cc(C(F)(F)F)c(-c3ccc(F)c4sc(N)nc34)c(F)c2n1] | LCMS m/z [M + 1]: 634.3 ¹HNMR (400 MHz, CD4O) δ 8.17 (s, 1H), 7.16-7.30 (m, 1H), 7.00 (t, J = 8.8 Hz, 1H), 4.89-4.98 (m, 1H), 4.70-4.81 (m, 3H), 4.21- 4.30 (m, 2H), 4.02-4.13 (m, 1H), 3.95 (d, J = 13.8 Hz, 2H), 3.44-3.59 (m, 1H), 3.10 (s, 3H), 1.90-2.28 (m, 6H), 1.27 (s, 3H), 1.22 (s, 3H). |
| 275 | 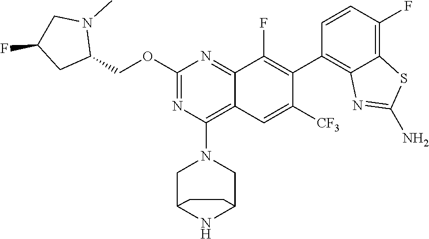 [SMILES: CN1C[C@H](F)C[C@H]1COc1nc(N2CC3CCC(C2)N3)c2cc(C(F)(F)F)c(-c3ccc(F)c4sc(N)nc34)c(F)c2n1] | LCMS m/z [M + 1]: 624.2 ¹HNMR (400 MHz, CD₃OD) δ 8.17 (s, 1H), 7.15-7.27 (m, 1H), 6.99 (t, J = 8.8 Hz, 1H), 5.47 (d, J = 52.4 Hz, 1H), 4.95-5.10 (m, 1H), 4.71-4.79 (m, 3H), 4.19-4.35 (m, 3H), 3.89- 4.19 (m, 3H), 3.56-3.78 (m, 1H), 3.18 (s, 3H), 2.50-2.79 (m, 1H), 2.35-2.48 (m, 1H), 1.85-2.28 (m, 4H). |

Additional compounds of the present disclosure are included in Table 9.

TABLE 9
Selected compounds of the present disclosure.

| Compound | | |
|---|---|---|
| No. | Structure | Analytical data |
| 151 | 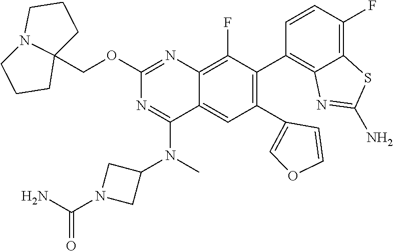 [SMILES: CN(c1nc(OCC23CCCN2CCC3)nc2c(F)c(-c3ccc(F)c4sc(N)nc34)c(-c3ccoc3)cc12)C1CN(C(N)=O)C1] | LCMS m/z [M + 1]: 647.1 |
| 201 | 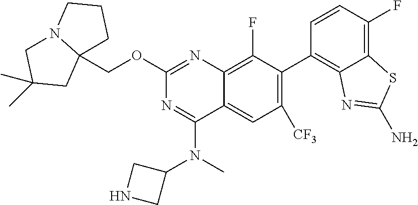 [SMILES: CN(c1nc(OCC23CCCN2CC(C)(C)C3)nc2c(F)c(-c3ccc(F)c4sc(N)nc34)c(C(F)(F)F)cc12)C1CNC1] | LCMS m/z [M + 1]: 634.2 |
| 281 | 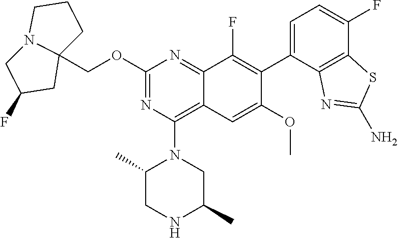 [SMILES: COc1cc2c(N3C[C@@H](C)NC[C@@H]3C)nc(OCC34CCCN3C[C@H](F)C4)nc2c(F)c1-c1ccc(F)c2sc(N)nc12] | LCMS m/z [M + 1]: 614.35 ¹HNMR (300 MHz, CD₃OD) δ 7.33- 7.18 (m, 2H), 7.06 (t, J = 9.2 Hz, 1H), 5.58 (d, J = 51.8 Hz, 1H), 4.69 (s, 2H), 4.38 (br s, 1H), 4.13-3.73 (m, 8H), 3.70- 3.35 (m, 4H), 2.82-2.56 (m, 2H), 2.48- 2.26 (m, 3H), 2.21-2.08 (m, 1H), 1.46 (dd, J = 6.5, 2.1 Hz, 3H), 1.41 (dd, J = 6.7, 2.4 Hz, 3H). |
| 282 | 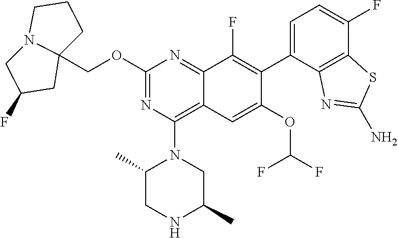 [SMILES: C[C@@H]1CN(c2nc(OCC34CCCN3C[C@H](F)C4)nc3c(F)c(-c4ccc(F)c5sc(N)nc45)c(OC(F)F)cc23)[C@@H](C)CN1] | LCMS m/z [M + 1]: 650.4 ¹HNMR (300 MHz, CD₃OD) δ 7.64 (s, 1H), 7.28 (dd, J = 8.4, 5.4 Hz, 1H), 7.03 (d, J = 8.8 Hz, 1H), 7.14-6.60 (m, 1H), 5.58 (d, J = 51.3 Hz, 1H), 4.70 (s, 2H), 4.46 (s, 1H), 4.04-3.68 (m, 8H), 3.48 (d, J = 7.0 Hz, 1H), 2.80-2.57 (m, 2H), 2.38 (dt, J = 17.6, 8.6 Hz, 3H), 2.19 (s, 1H), 1.51 (dd, J = 6.6, 3.9 Hz, 3H), 1.45- 1.38 (m, 3H). |
| 283 | 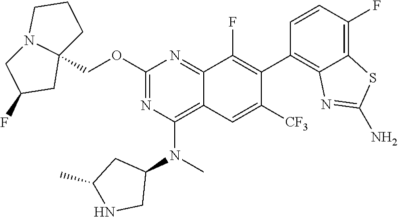 [SMILES: C[C@@H]1C[C@@H](N(C)c2nc(OC[C@@]34CCCN3C[C@H](F)C4)nc3c(F)c(-c4ccc(F)c5sc(N)nc45)c(C(F)(F)F)cc23)CN1] | LCMS m/z [M + 1]: 652.3 ¹HNMR (400 MHz, CD₃OD) δ 8.37 (s, 1H), 7.21 (dd, J = 8.0, 6.0 Hz, 1H), 7.00 (t, J = 8.8 Hz, 1H), 5.55 (d, J = 51.6 Hz, 1H), 5.43-5.48 (m, 1H), 4.64-4.78 (m, 2H), 3.83-4.19 (m, 5H), 3.53 (s, 3H), 3.41-3.51 (m, 2H), 2.57-2.74 (m, 3H), 2.12-2.47 (m, 5H), 1.47 (d, J = 6.4 Hz, 3H). |
| 284 | 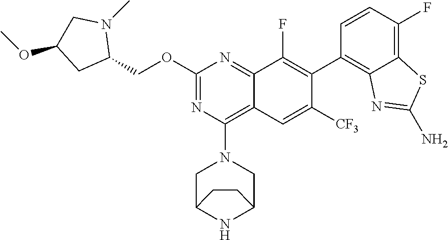 [SMILES: CO[C@@H]1C[C@@H](COc2nc(N3CC4CCC(C3)N4)c3cc(C(F)(F)F)c(-c4ccc(F)c5sc(N)nc45)c(F)c3n2)N(C)C1] | LCMS m/z [M + 1]: 636.3 ¹HNMR (400 MHz, CD₃OD) δ 8.17 (s, 1H), 7.16-7.26 (m, 1H), 7.00 (t, J = 8.8 Hz, 1H), 4.61-5.00 (m, 5H), 4.04-4.30 (m, 4H), 3.79-4.00 (m, 3H), 3.39 (s, 3H), 3.14 (s, 3H), 2.47-2.66 (m, 1H), 1.96-2.24 (m, 5H). |
| 285 | 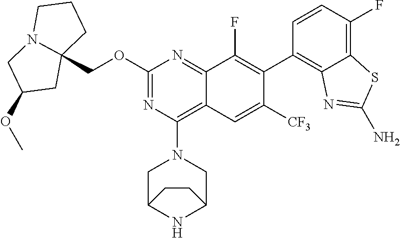 [SMILES: CO[C@H]1CN2CCC[C@]2(COc2nc(N3CC4CCC(C3)N4)c3cc(C(F)(F)F)c(-c4ccc(F)c5sc(N)nc45)c(F)c3n2)C1] | LCMS m/z [M + 1]: 662.2 ¹HNMR (400 MHz, CD₃OD) δ 8.19 (s, 1H), 7.20-7.25 (m, 1H), 7.02(t, J = 8.8 Hz, 1H), 4.84-4.87(m, 1H), 4.76- 4.84(m, 2H), 4.62-4.71 (m, 2H), 4.26- 4.33(m, 2H), 3.96-4.02 (m, 2H), 3.76-3.88 (m, 2H), 3.65-3.69 (m, 1H), 3.49-3.59 (m, 1H) 3.41 (s, 3H), 2.43-2.52 (m, 2H) 2.27-2.40 (m, 3H), 2.08-2.19 (m, 5H). |
| 286 | 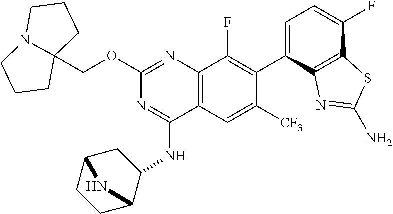 | LCMS m/z [M + 1]: 632.2 ¹HNMR (500 MHz, CD₃OD) δ 8.74 (s, 1H), 7.28-7.17 (m, 1H), 7.04-6.96 (m, 1H), 4.72 (t, J = 4.6 Hz, 1H), 4.69 (s, 2H), 4.33 (t, J = 4.8 Hz, 1H), 3.73-3.63 (m, 2H), 3.29-3.25 (m, 1H), 2.61-2.52 (m, 1H), 2.38-2.28 (m, 2H), 2.28-2.18 (m, 2H), 2.18-2.07 (m, 6H), 2.06-1.97 (m, 4H), 1.92 (d, J = 15.5 Hz, 1H). |
| 287 | 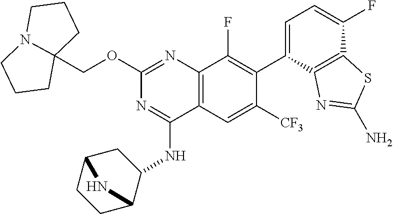 | LCMS m/z [M + 1]: 632.2 ¹HNMR (500 MHz, CD₃OD) δ 8.73 (s, 1H), 7.26-7.18 (m, 1H), 7.01 (dd, J = 9.2, 8.4 Hz, 1H), 4.72 (t, J = 4.7 Hz, 1H), 4.69 (s, 2H), 4.33 (t, J = 4.8 Hz, 1H), 3.68 (tt, J = 11.1, 6.6 Hz, 2H), 3.28 (s, 2H), 2.61-2.51 (m, 1H), 2.39-2.27 (m, 2H), 2.23 (dqt, J = 13.4, 9.2, 5.2 Hz, 3H), 2.12 (dp, J = 13.2, 6.3, 5.8 Hz, 6H), 2.09-1.97 (m, 4H), 1.93 (d, J = 11.9 Hz, 2H). |
| 288 | 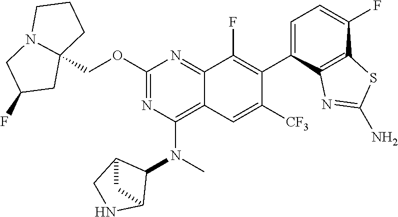 | LCMS m/z [M + 1]: 650.3 ¹HNMR (400 MHz, CD₃OD) δ 8.39 (s, 1H), 7.19 (dd, J = 8.0, 5.2 Hz, 1H), 7.00 (t, J = 8.8 Hz, 1H), 5.56 (d, J = 52 Hz, 1H), 4.65-4.77 (m, 3H), 3.99-4.06 (m, 1H), 3.87-3.97 (m, 2H), 3.33-3.56 (m, 8H), 2.57-2.74 (m, 2H), 2.30-2.48 (m, 3H), 2.12-2.24 (m, 1H), 2.04-2.10 (m, 1H), 1.59 (d, J = 9.6 Hz, 1H). |
| 289 | 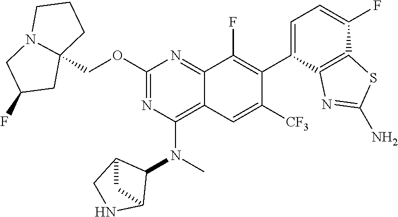 | LCMS m/z [M + 1]: 650.3 ¹HNMR (400 MHz, CD₃OD) δ 8.41 (s, 1H), 7.19 (dd, J = 8.0, 5.6 Hz, 1H), 6.99 (t, J = 8.8 Hz, 1H), 5.57 (d, J = 51.2 Hz, 2H), 4.65-4.78 (m, 3H), 3.97-4.05 (m, 1H), 3.87-3.96 (m, 2H), 3.32-3.63 (m, 8H), 2.55-2.74 (m, 2H), 2.32-2.52 (m, 3H), 2.13-2.23 (m, 1H), 2.02-2.10 (m, 1H), 1.60 (d, J = 9.2 Hz, 1H). |
| 290 | 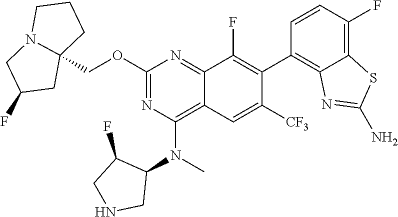 [SMILES: CN(c1nc(OC[C@@]23CCCN2C[C@H](F)C3)nc2c(F)c(-c3ccc(F)c4sc(N)nc34)c(C(F)(F)F)cc12)[C@H]1CNC[C@H]1F] | LCMS m/z [M + 1]: 656.2 ¹HNMR (400 MHz, CD₃OD) δ 8.41 (s, 1H), 7.20-7.23 (m, 1H), 7.00 (t, J = 8.8 Hz, 1H), 5.85 (d, J = 51.6 Hz, 1H), 5.55 (d, J = 52 Hz, 1H), 5.24-5.36 (m, 1H), 4.64-4.75 (m, 2H), 3.73-4.06 (m, 7H), 3.66 (s, 3H), 3.43-3.49 (m, 1H), 2.15-2.72 (m, 6H). |
| 291 | 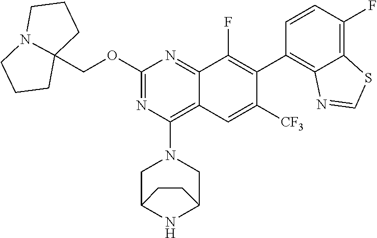 [SMILES: Fc1c(-c2ccc(F)c3scnc23)c(C(F)(F)F)cc2c(N3CC4CCC(C3)N4)nc(OCC34CCCN3CCC4)nc12] | LCMS m/z [M + 1]: 617.2 ¹HNMR (400 MHz, CD₃OD) δ 9.27 (s, 1H), 8.22 (s, 1H), 7.55-7.59 (m, 1H), 7.42-7.46 (m, 1H), 4.76-4.79 (m, 3H), 4.68 (s, 2H), 4.26 (s, 2H), 3.94-4.01 (m, 2H), 3.67-3.70 (m, 2H), 3.31 (m 1H), 2.09-2.35 (m, 12H). |
| 292 | 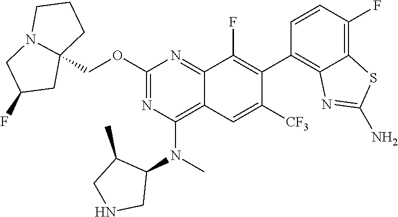 [SMILES: C[C@@H]1CNC[C@@H]1N(C)c1nc(OC[C@@]23CCCN2C[C@H](F)C3)nc2c(F)c(-c3ccc(F)c4sc(N)nc34)c(C(F)(F)F)cc12] | LCMS m/z [M + 1]: 652.2 ¹HNMR(400 MHz, CD₃OD) δ 8.40 (s, 1H), 7.17-7.24 (m,1H), 6.99 (t, J = 8.8 Hz, 1H), 5.54 (d, J = 50.8 Hz, 1H), 5.14-5.22 (m, 1H), 4.63-4.78 (m, 2H), 3.80-4.01 (m, 5H), 3.62-3.71 (m, 1H), 3.61 (s, 3H), 3.39-3.50 (m, 2H ), 3.01-3.15 (m, 1H), 2.52-2.67 (m, 2H), 2.25-2.50 (m, 3H), 2.05-2.21 (m, 1H), 1.14 (d, J = 7.2 Hz, 3H). |
| 293 | 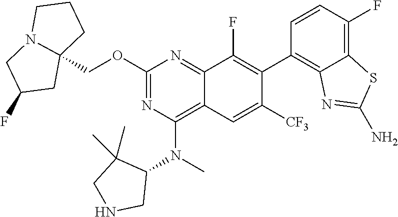 | LCMS m/z [M + 1]: 663.3 ¹HNMR (400 MHz, CD₃OD) δ 8.44 (s, 1H), 7.18-7.29 (m, 1H), 7.01 (t, J = 8.8 Hz, 1H), 5.45-5.65 (d, J = 50.8 Hz, 1H), 4.95-5.11 (m, 1H), 4.63-4.75 (m, 2H), 3.80-4.03 (m, 5H), 3.52-3.71 (m, 4H), 3.40-3.50 (m, 1H), 3.30 (s, 1H), 2.50-2.78 (m, 2H), 2.24-2.50 (m, 3H), 2.02-2.21 (m, 1H), 1.41 (s, 3H), 1.24 (s, 3H). |
| 294 | 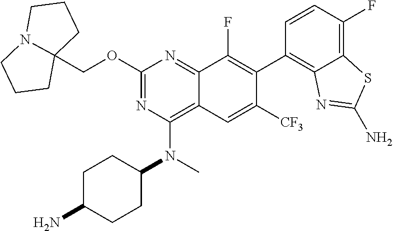 | LCMS m/z [M + 1]: 648.3 ¹HNMR (400 MHz, CD₃OD) δ 8.34 (s, 1H), 7.23-7.26 (m, 1H), 7.02 (t, J = 8.8 Hz, 1H), 4.53-4.68 (m, 3H), 3.65-3.74 (m, 2H), 3.59-3.64 (m, 1H), 3.50 (s, 3H), 3.18-3.41 (m, 2H), 2.03-2.34 (m, 16H). |
| 295 | 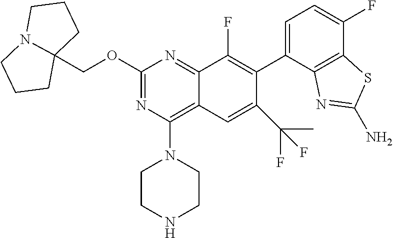 [SMILES: CC(F)(F)c1cc2c(N3CCNCC3)nc(OCC34CCCN3CCC4)nc2c(F)c1-c1ccc(F)c2sc(N)nc12] | LCMS m/z [M + 1]: 602.34 ¹HNMR (300 MHz, CD₃OD) δ 8.05 (d, J = 21.1 Hz, 1H), 7.23 (ddd, J = 8.0, 5.4, 2.3 Hz, 1H), 7.03 (dd, J = 9.2, 8.5 Hz, 1H), 4.70 (s, 2H), 4.21 (t, J = 5.3 Hz, 4H), 3.69 (dq, J = 13.6, 6.1 Hz, 2H), 3.56-3.48 (m, 4H), 3.28 (d, J = 6.6 Hz, 2H), 2.22 (dddt, J = 43.6, 18.7, 12.0, 5.6 Hz, 8H), 1.66 (dd, J = 19.3, 18.1 Hz, 3H). |
| 296 | 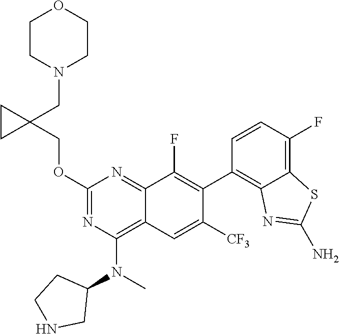 [SMILES: CN(c1nc(OCC2(CN3CCOCC3)CC2)nc2c(F)c(-c3ccc(F)c4sc(N)nc34)c(C(F)(F)F)cc12)[C@@H]1CCNC1] | LCMS m/z [M + 1]: 650.2 ¹HNMR (400 MHz, CD₃OD) δ 8.34 (s, 1H), 7.20-7.24 (m, 1H), 7.00 (t, J = 8.8 Hz, 1H), 5.26-5.34 (m, 1H), 4.58 (d, J = 12 Hz, 1H), 4.40 (d, J = 12 Hz, 1H), 3.69- 4.04 (m, 8H), 3.35-3.51 (m, 7H), 3.13- 3.23 (m, 2H), 2.37-2.55 (m, 2H), 0.88- 0.99 (m, 4H). |
| 297 | 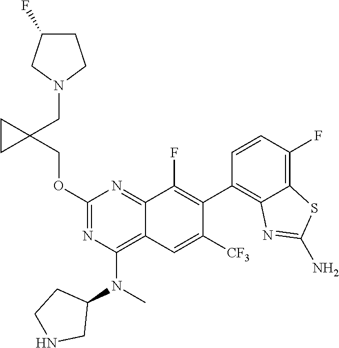 [SMILES: CN(c1nc(OCC2(CN3CC[C@@H](F)C3)CC2)nc2c(F)c(-c3ccc(F)c4sc(N)nc34)c(C(F)(F)F)cc12)[C@@H]1CCNC1] | LCMS m/z [M + 1]: 652.3 ¹HNMR (400 MHz, CD₃OD) δ 8.36 (s, 1H), 7.21-7.25 (m, 1H), 7.01 (t, J = 8.8 Hz, 1H), 5.28-5.51 (m, 2H), 4.64 (d, J = 11.6 Hz, 1H), 4.34 (d, J = 12.4 Hz, 1H), 3.40-3.99 (m, 13H), 2.39-2.60 (m, 4H), 0.86-0.98 (m, 4H). |
| 299 | 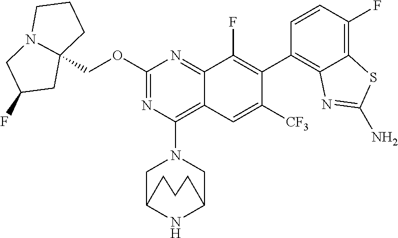 [SMILES: Nc1nc2c(-c3c(C(F)(F)F)cc4c(N5CC6CCCC(C5)N6)nc(OC[C@@]56CCCN5C[C@H](F)C6)nc4c3F)ccc(F)c2s1] | LCMS m/z [M + 1]: 664.3 ¹HNMR (400 MHz, CD₃OD) δ 8.24 (s, 1H), 7.23 (dd, J = 8.8, 5.6 Hz, 1H), 7.01 (t, J = 8.8 Hz, 1H), 5.57 (d, J = 51.6 Hz, 1H), 4.89-4.95 (s, 2H), 4.68-4.77 (m, 2H), 3.82-4.09 (m, 7H), 3.43-3.53 (m, 1H), 2.56-2.78 (m, 2H), 2.31-2.78 (m, 3H), 2.04-2.26 (m, 6H), 1.66-1.75 (m, 1H). |
| 300 | 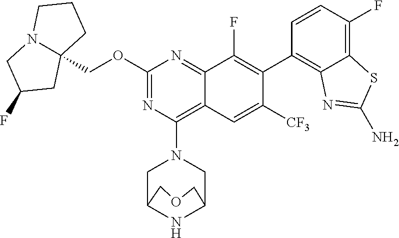 [SMILES: Nc1nc2c(-c3c(C(F)(F)F)cc4c(N5CC6COCC(C5)N6)nc(OC[C@@]56CCCN5C[C@H](F)C6)nc4c3F)ccc(F)c2s1] | LCMS m/z [M + 1]: 666.2 ¹HNMR (400 MHz, CD₃OD) δ 8.23 (s, 1H), 7.21 (dd, J = 8.8, 6.0 Hz, 1H), 7.00 (t, J = 8.8 Hz, 1H), 5.57 (d, J = 51.2 Hz, 1H), 5.19 (dd, J = 21.6, 14.0 Hz, 2H), 4.64-4.76 (m, 2H), 3.83-4.28 (m, 8H), 3.72 (s, 2H), 3.42-3.51 (m, 1H), 3.21-3.25 (m, 1H), 2.58-2.73 (m, 2H), 2.30-2.47 (m, 3H), 2.11-2.20 (m, 1H). |
| 301 | 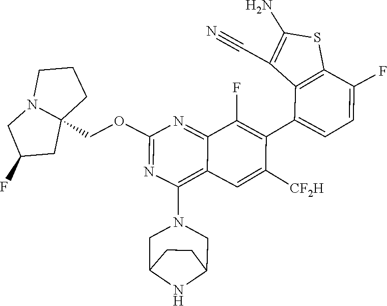 [SMILES: N#Cc1c(N)sc2c(F)ccc(-c3c(C(F)F)cc4c(N5CC6CCC(C5)N6)nc(OC[C@@]56CCCN5C[C@H](F)C6)nc4c3F)c12] | LCMS m/z [M + 1]: 656.32 ¹H NMR (300 MHz, CD₃OD) δ 8.05 (s, 1H), 7.23 (dd, J = 8.4, 5.0 Hz, 1H), 7.05 (dd, J = 9.5, 8.4 Hz, 1H), 6.56 (t, J = 54.8 Hz, 1H), 5.43 (d, J = 52.9 Hz, 1H), 4.67- 4.52 (m, 2H), 4.53-4.40 (m, 2H), 3.97- 3.89 (m, 2H), 3.85-3.67 (m, 3H), 3.64- 3.51 (m, 3H), 2.55-2.35 (m, 2H), 2.32- 2.11 (m, 4H), 1.97 (s, 4H). |
| 302 | 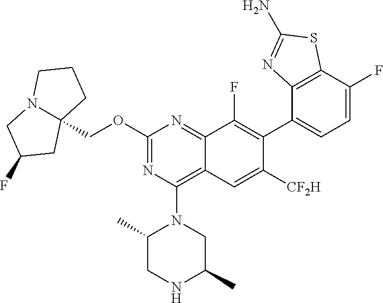 [SMILES: C[C@@H]1CN(c2nc(OC[C@@]34CCCN3C[C@H](F)C4)nc3c(F)c(-c4ccc(F)c5sc(N)nc45)c(C(F)F)cc23)[C@@H](C)CN1] | LCMS m/z [M + 1]: 634.35 ¹H NMR (300 MHz, CD₃OD) δ 8.16 (s, 1H), 7.26 (t, J = 7.1 Hz, 1H), 7.04 (t, J = 8.8 Hz, 1H), 6.62 (t, J = 55.0 Hz, 1H), 5.58 (d, J = 53.9 Hz, 1H), 4.72 (s, 2H), 4.65 (s, 1H), 4.06-3.97 (m, 3H), 3.98- 3.82 (m, 4H), 3.73-3.57 (m, 2H), 3.52- 3.42 (m, 1H), 2.79-2.57 (m, 2H), 2.47- 2.21 (m, 4H), 1.57 (d, J = 6.7 Hz, 3H), 1.46 (d, J = 6.7 Hz, 3H). |
| 304 | 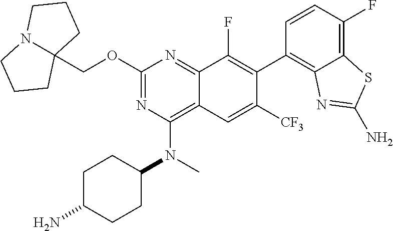 [SMILES: CN(c1nc(OCC23CCCN2CCC3)nc2c(F)c(-c3ccc(F)c4sc(N)nc34)c(C(F)(F)F)cc12)[C@H]1CC[C@H](N)CC1] | LCMS m/z [M + 1]: 648.3 ¹HNMR (400 MHz, CD₃OD) δ 8.31 (s, 1H), 7.17-7.29 (m, 1H), 6.99 (t, J = 8.8 Hz, 1H), 4.67 (s, 2H), 3.62-3.73 (m, 2H), 3.41 (s, 3H), 3.16-3.30 (m, 4H), 1.84-2.58 (m, 14H), 1.58-1.74 (m, 2H). |
| 307 | 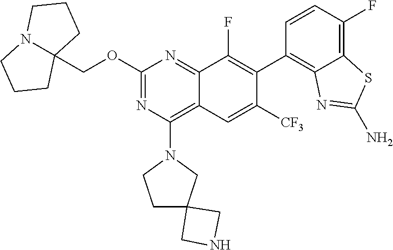 [SMILES: Nc1nc2c(-c3c(C(F)(F)F)cc4c(N5CCC6(CNC6)C5)nc(OCC56CCCN5CCC6)nc4c3F)ccc(F)c2s1] | LCMS m/z [M + 1]: 632.2 ¹HNMR (500 MHz, CD₃OD) δ 8.49 (s, 1H), 7.36 (dd, J = 8.6, 5.1 Hz, 1H), 7.15 (t, J = 8.8 Hz, 1H), 4.84-4.73 (m, 2H), 4.43 (s, 2H), 4.36-4.11 (m, 7H), 3.73 (dtd, J = 14.4, 6.8, 3.1 Hz, 2H), 3.30- 3.26 (m, 1H), 2.53 (t, J = 7.0 Hz, 2H), 2.41-2.06 (m, 8H). |
| 308 | 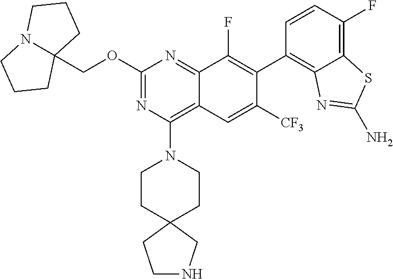 [SMILES: Nc1nc2c(-c3c(C(F)(F)F)cc4c(N5CCC6(CCNC6)CC5)nc(OCC56CCCN5CCC6)nc4c3F)ccc(F)c2s1] | LCMS m/z [M + 1]: 660.2 ¹HNMR (500 MHz, CD₃OD) δ 8.21 (s, 1H), 7.28 (dd, J = 8.7, 5.2 Hz, 1H), 7.07 (t, J = 8.8 Hz, 1H), 4.75-4.64 (m, 2H), 4.20-3.96 (m, 5H), 3.75-3.64 (m, 2H), 3.48 (t, J = 7.5 Hz, 2H), 3.28 (s, 3H), 2.39- 2.05 (m, 10H), 1.93 (ddqt, J = 15.8, 12.5, 8.3, 4.0 Hz, 4H). |
| 309 | 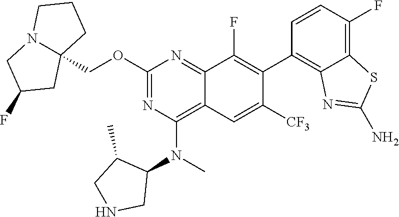 [SMILES: C[C@H]1CNC[C@@H]1N(C)c1nc(OC[C@@]23CCCN2C[C@H](F)C3)nc2c(F)c(-c3ccc(F)c4sc(N)nc34)c(C(F)(F)F)cc12] | LCMS m/z [M + 1]: 652.3 ¹HNMR (400 MHz, CD₃OD) δ 8.40 (s, 1H), 7.23-7.26 (m, 1H), 6.05 (t, J = 8.8 Hz, 1H), 5.56 (d, J = 51.6 Hz, 1H), 4.99- 5.01 (m, 1H), 4.69-4.72 (m, 2H), 3.86- 3.92 (m, 4H), 3.69-3.73 (m, 1H), 3.38- 3.53 (m, 5H), 3.03-3.06 (m, 1H), 2.30- 2.83 (m, 7H), 1.25-1.27 (m, 3H). |
| 311 | 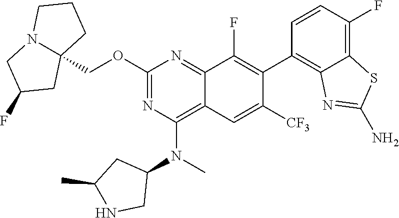 [SMILES: C[C@H]1C[C@@H](N(C)c2nc(OC[C@@]34CCCN3C[C@H](F)C4)nc3c(F)c(-c4ccc(F)c5sc(N)nc45)c(C(F)(F)F)cc23)CN1] | LCMS m/z [M + 1]: 652.3 ¹HNMR (400 MHz, CD₃OD) δ 8.36 (s, 1H), 7.19-7.27 (m, 1H), 7.00 (t, J = 8.0 Hz, 1H), 5.55 (d, J = 52.4 Hz, 1H), 5.28-5.39 (m, 1H), 4.71 (s, 2H), 3.79-4.01 (m, 5H), 3.55 (s, 3H), 3.42-3.51 (m, 2H), 2.58-2.86 (m, 3H), 2.30-2.51 (m, 3H), 2.08-2.22 (m, 2H), 1.58 (d, J = 6.0 Hz, 3H). |
| 312 | 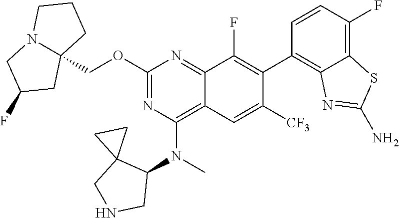 [SMILES: CN(c1nc(OC[C@@]23CCCN2C[C@H](F)C3)nc2c(F)c(-c3ccc(F)c4sc(N)nc34)c(C(F)(F)F)cc12)[C@H]1CNCC12CC2] | LCMS m/z [M + 1]: 664.3 ¹HNMR (400 MHz, CD₃OD) δ 8.37 (s, 1H), 7.19-7.23 (m, 1H), 6.98 (t, J = 8.8 Hz, 1H), 5.55 (d, J = 51.2 Hz, 1H), 5.17- 5.30 (m, 1H), 3.75-4.12 (m, 6H), 3.61 (s, 3H), 3.41-3.50 (m, 1H), 3.21-3.30 (m, 1H), 2.56-2.69 (m, 2H), 2.30-2.50 (m, 3H), 2.09-2.21 (m, 1H), 0.91-1.29 (m, 4H). |
| 313 | 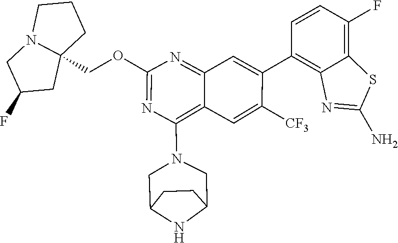 [SMILES: Nc1nc2c(-c3cc4nc(OC[C@@]56CCCN5C[C@H](F)C6)nc(N5CC6CCC(C5)N6)c4cc3C(F)(F)F)ccc(F)c2s1] | LCMS m/z [M + 1]: 632.3 ¹HNMR (400 MHz, CD₃OD) δ 8.38 (s, 1H), 7.64 (s, 1H), 7.15-7.31 (m, 1H), 7.01-7.03 (m, 1H), 5.53 (d, J = 51.2 Hz, 1H), 4.65-4.83 (m, 4H), 4.27 (s, 2H), 3.80-4.12 (m, 5H), 3.41-3.54 (m, 1H), 2.06-2.76 (m, 10H). |
| 314 | 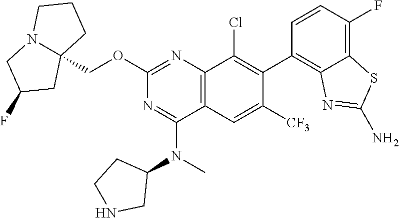 [SMILES: CN(c1nc(OC[C@@]23CCCN2C[C@H](F)C3)nc2c(Cl)c(-c3ccc(F)c4sc(N)nc34)c(C(F)(F)F)cc12)[C@@H]1CCNC1] | LCMS m/z [M + 1]: 654.3 ¹HNMR (400 MHz, CD₃OD) δ 8.49 (s, 1H), 7.10-7.21 (m, 1H), 7.01 (t, J = 8.8 Hz, 1H), 5.55 (d, J = 52 Hz, 1H), 5.25- 5.29 (m, 1H), 4.68-4.78 (m, 2H), 3.84- 4.02 (m, 3H), 3.70-3.71 (m, 1H), 3.53 (s, 3H), 3.44-3.49 (m, 3H), 2.33-2.69 (m, 8H), 2.08-2.21(s, 1H). |
| 315 | 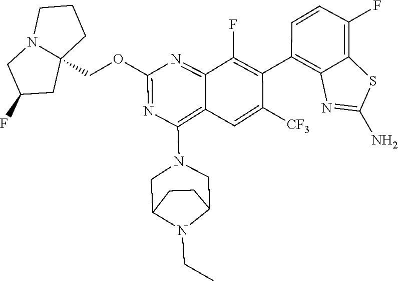 [SMILES: CCN1C2CCC1CN(c1nc(OC[C@@]34CCCN3C[C@H](F)C4)nc3c(F)c(-c4ccc(F)c5sc(N)nc45)c(C(F)(F)F)cc13)C2] | LCMS m/z [M + 1]: 678.4 ¹HNMR (400 MHz, CD₃OD) δ 8.17 (s, 1H), 7.17-7.24 (m, 1H), 7.00(t, J = 8.8 Hz, 1H), 5.57 (d, J = 52 Hz, 1H), 4.78- 4.83 (m, 3H), 4.64-4.75 ( m, 2H), 4.19- 4.32 (m, 2H), 3.82-4.10 (m, 5H), 3.42- 3.52 (m, 1H), 3.17-3.27 (m, 1H), 2.54- 2.76 (m, 2H) 2.08-2.50 (m, 8H), 1.40-1.52 (m, 3H). |
| 316 | 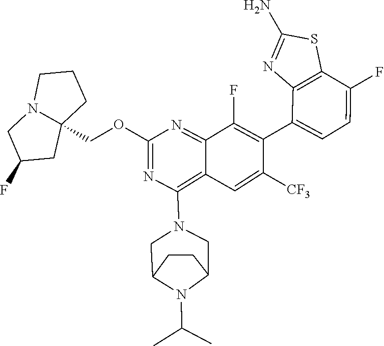 [SMILES: CC(C)N1C2CCC1CN(c1nc(OC[C@@]34CCCN3C[C@H](F)C4)nc3c(F)c(-c4ccc(F)c5sc(N)nc45)c(C(F)(F)F)cc13)C2] | LCMS m/z [M + 1]: 692.4 ¹HNMR (400 MHz, CD₃OD) δ 8.18 (s, 1H), 7.18-7.25 (m, 1H), 7.01 (t, J = 9.2 Hz, 1H), 5.45 (d, J = 52 Hz, 1H), 4.78- 4.88 (m, 2H), 4.68-4.72 (m, 2H), 4.22- 4.52 (m, 2H), 3.83-3.11 (m, 5H), 3.42- 3.52 (m, 1H), 3.15-3.25 (m, 1H), 2.53- 2.76 (m, 2H), 2.02-2.47 (m, 8H), 1.43- 1.54 (m, 6H). |
| 318 | 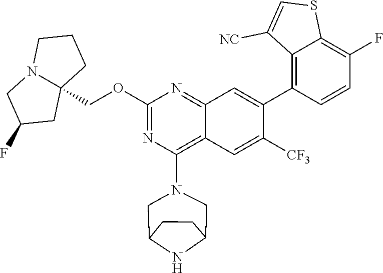 [SMILES: N#Cc1csc2c(F)ccc(-c3cc4nc(OC[C@@]56CCCN5C[C@H](F)C6)nc(N5CC6CCC(C5)N6)c4cc3C(F)(F)F)c12] | LCMS m/z [M + 1]: 641.3 ¹HNMR (400 MHz, CD₃OD) δ 8.65 (s, 1H), 8.38 (s, 1H), 7.69 (s, 1H), 7.51-7.53 (m, 1H), 7.42 (t, J = 9.2 Hz, 1H), 5.58 (d, J = 52 Hz, 1H), 4.62-4.72 (m, 3H), 4.21- 4.29 (m, 2H), 3.88-4.06 (m, 6H), 3.41- 3.52 (m, 1H), 2.02-2.72 (m, 10H). |
| 319 | 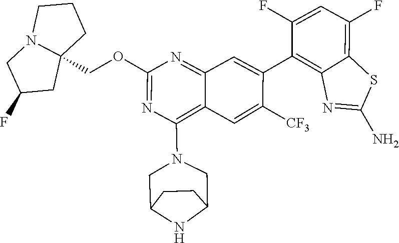 [SMILES: Nc1nc2c(-c3cc4nc(OC[C@@]56CCCN5C[C@H](F)C6)nc(N5CC6CCC(C5)N6)c4cc3C(F)(F)F)c(F)cc(F)c2s1] | LCMS m/z [M + 1]: 650.3 ¹HNMR (400 MHz, CD₃OD) δ 8.37 (s, 1H), 7.64 (s, 1H), 6.88 (t, J = 8.8 Hz, 1H), 5.59 (d, J = 51.2 Hz, 1H), 4.63-4.84 (m, 4H), 4.25 (s, 2H), 3.86-3.99 (m, 5H), 3.44-3.51 (m, 1H), 2.58-2.73 (m, 2H), 2.31-2.45 (m, 3H), 2.05-2.19 (m, 5H). |
| 320 | 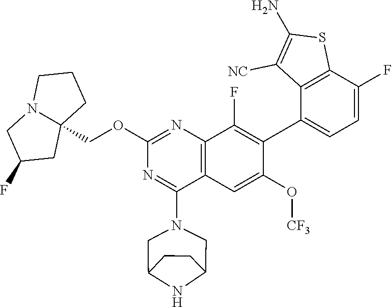 [SMILES: N#Cc1c(N)sc2c(F)ccc(-c3c(OC(F)(F)F)cc4c(N5CC6CCC(C5)N6)nc(OC[C@@]56CCCN5C[C@H](F)C6)nc4c3F)c12] | LCMS m/z [M + 1]: 690.3 ¹HNMR (400 MHz, CD₃OD) δ 7.79 (s, 1H), 7.25 (dd, J = 8.4, 4.8 Hz, 1H), 7.06 (t, J = 8.8 Hz, 1H), 5.57 (d, J = 52.4 Hz, 1H), 4.62-4.76 (m, 4H), 4.25 (s, 2H), 3.84-4.07 (m, 5H), 3.47-3.48 (m, 1H), 2.55-2.74 (m, 2H), 2.35-2.45 (m, 3H), 2.09-2.12 (m, 5H). |
| 322 | 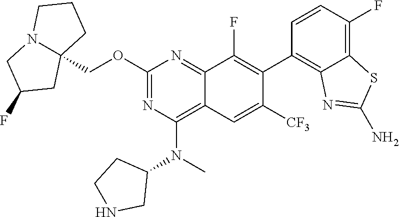 [SMILES: CN(c1nc(OC[C@@]23CCCN2C[C@H](F)C3)nc2c(F)c(-c3ccc(F)c4sc(N)nc34)c(C(F)(F)F)cc12)[C@H]1CCNC1] | LCMS m/z [M + 1]: 638.3 ¹HNMR (400 MHz, CD₃OD) δ 8.37 (s, 1H), 7.22 (dd, J = 8.4, 5.6 Hz, 1H), 7.01 (t, J = 8.8 Hz, 1H), 5.55 (d, J = 52.0 Hz, 1H), 5.28-5.37 (m, 1H), 4.55-4.79 (m, 2H), 3.81-4.06 (m, 4H), 3.61-3.73 (m, 1H), 3.54 (s, 3H), 3.37-3.51 (m, 3H), 2.52-2.77 (m, 3H), 2.26-2.49 (m, 4H), 2.09-2.12 (m, 1H). |
| 323 | 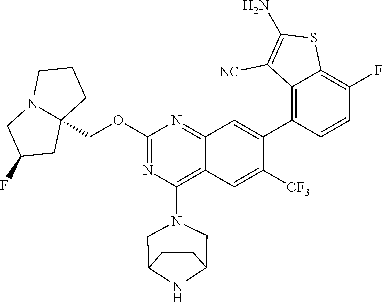 [SMILES: N#Cc1c(N)sc2c(F)ccc(-c3cc4nc(OC[C@@]56CCCN5C[C@H](F)C6)nc(N5CC6CCC(C5)N6)c4cc3C(F)(F)F)c12] | LCMS m/z [M + 1]: 656.3 ¹HNMR (400 MHz, CD₃OD) δ 8.32 (s, 1H), 7.60 (s, 1H), 7.17-7.21 (m, 1H), 6.99 (t, J = 8.8 Hz, 1H), 5.58(d, J = 52.8 Hz, 1H), 4.79-4.85 (m, 1H), 4.61-4.72 (m, 3H), 4.23-4.24 (m, 2H), 3.85-4.01 (m, 5H), 3.47-3.50 (m, 1H), 2.58-2.71 (m, 2H), 2.32-2.46 (m, 3H), 2.10-2.19 (m, 5H). |
| 324 | 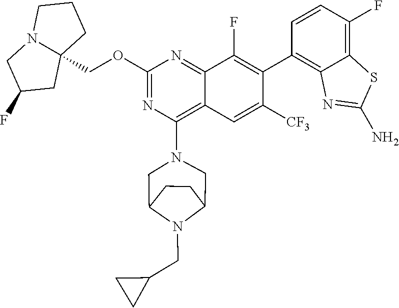 [SMILES: Nc1nc2c(-c3c(C(F)(F)F)cc4c(N5CC6CCC(C5)N6CC5CC5)nc(OC[C@@]56CCCN5C[C@H](F)C6)nc4c3F)ccc(F)c2s1] | LCMS m/z [M + 1]: 704.4 ¹HNMR (400 MHz, CD₃OD) δ 8.18 (s, 1H), 7.19-7.25 (m, 1H), 7.01 (t, J = 8.8 Hz, 1H), 5.57 (d, J = 51.6 Hz, 1H), 4.79- 4.84 (m, 3H), 4.65-4.78 (m, 2H), 4.31- 4.43 (m, 2H), 3.82-4.15 (m, 5H), 3.15- 3.43 (m, 1H), 3.03-3.13 (m, 1H), 2.54- 2.79 (m, 2H), 2.06-2.47 (m, 8H) 1.16-1.26 (m, 1H), 0.79-0.86 (m, 2H), 0.47-0.55 (m, 2H). |
| 325 | 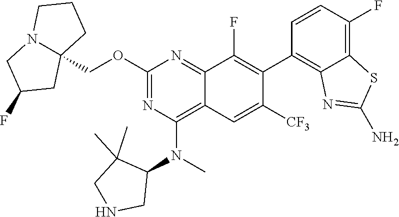 [SMILES: CN(c1nc(OC[C@@]23CCCN2C[C@H](F)C3)nc2c(F)c(-c3ccc(F)c4sc(N)nc34)c(C(F)(F)F)cc12)[C@H]1CNCC1(C)C] | LCMS m/z [M + 1]: 665.4 ¹HNMR (400 MHz, CD₃OD) δ 8.44 (s, 1H), 7.19-7.22 (m, 1H), 6.99 (t, J = 9.2 Hz, 1H), 5.53 (d, J = 50.8 Hz, 1H), 5.03- 5.11 (m, 1H), 4.69-4.71 (m, 2H), 3.62- 3.99 (m, 10H), 3.45-3.48 (m, 1H), 2.46- 2.64 (m, 2H), 2.15-2.38 (m, 4H), 1.40 (s, 3H), 1.23 (s, 3H). |
| 326 | 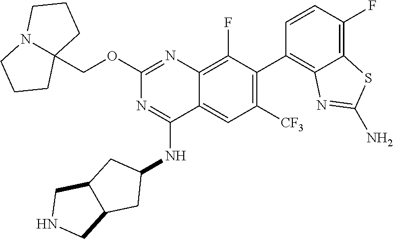 [SMILES: Nc1nc2c(-c3c(C(F)(F)F)cc4c(N[C@H]5C[C@H]6CNC[C@H]6C5)nc(OCC56CCCN5CCC6)nc4c3F)ccc(F)c2s1] | LCMS m/z [M + 1]: 646.1 ¹HNMR (400 MHz, CD₃OD) 8.69 (s, 1H), 7.26-7.29 (m, 1H), 7.06 (t, 1H), 4.74- 4.80 (m, 3H), 3.71 (s, 2H), 3.48 (s, 2H), 3.27-3.32 (m, 3H), 3.04 (s, 2H), 2.61 (s, 2H), 2.10-2.37 (m, 8H), 1.68 (s, 2H), 1.30 (s, 1H). |
| 327 | 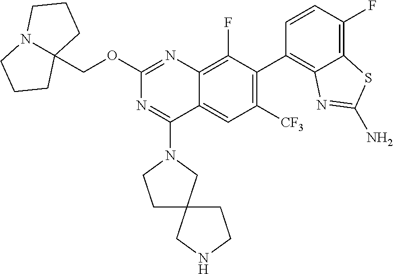 [SMILES: Nc1nc2c(-c3c(C(F)(F)F)cc4c(N5CCC6(CCNC6)C5)nc(OCC56CCCN5CCC6)nc4c3F)ccc(F)c2s1] | LCMS m/z [M + 1]: 646.2 ¹H NMR (500 MHz, CD₃OD) δ 8.50 (s, 1H), 7.32 (dd, J = 8.6, 5.2 Hz, 1H), 7.16- 7.08 (m, 1H), 4.76 (q, J = 12.2 Hz, 2H), 4.31 (s, 3H), 4.21 (s, 3H), 3.72 (dt, J = 11.9, 6.5 Hz, 2H), 3.58-3.36 (m, 4H), 3.29 (d, J = 5.8 Hz, 2H), 2.40-2.07 (m, 10H). |
| 328 | 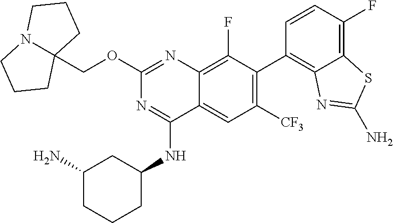 [SMILES: Nc1nc2c(-c3c(C(F)(F)F)cc4c(N[C@H]5CCC[C@H](N)C5)nc(OCC56CCCN5CCC6)nc4c3F)ccc(F)c2s1] | LCMS m/z [M + 1]: 634 ¹HNMR (400 MHz, CD₃OD) δ 8.70 (s, 1H), 7.21-7.24 (m, 1H), 7.00-7.04 (m, 1H), 4.70-4.71 (m, 3H), 3.66-3.69 (m, 3H), 3.21-3.32 (m, 3H), 1.86-2.33 (m, 15H). |
| 329 | 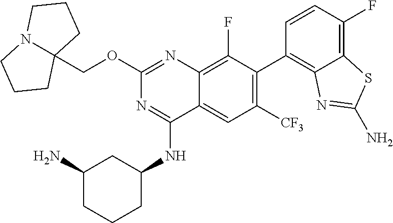 [SMILES: Nc1nc2c(-c3c(C(F)(F)F)cc4c(N[C@H]5CCC[C@@H](N)C5)nc(OCC56CCCN5CCC6)nc4c3F)ccc(F)c2s1] | LCMS m/z [M + 1]: 634 ¹HNMR (400 MHz, CD₃OD) 8.65 (s, 1H), 7.24-7.28 (m, 1H), 7.03-7.07 (m, 1H), 4.71-4.72 (m, 2H), 4.78-4.79 (m, 1H), 3.70-3.72 (m, 2H), 3.27-3.28 (m, 3H), 2.48-2.51 (m, 1H), 2.31-2.35 (m, 2H), 2.22-2.25 (m, 2H), 2.04-2.22 (m, 6H), 1.44-1.65 (m, 5H). |
| 330 | 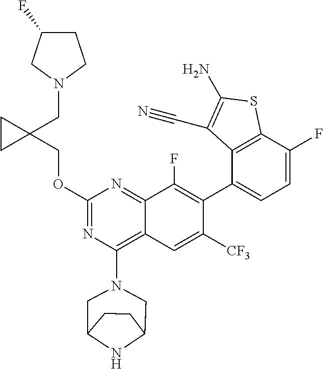 [SMILES: N#Cc1c(N)sc2c(F)ccc(-c3c(C(F)(F)F)cc4c(N5CC6CCC(C5)N6)nc(OCC5(CN6CC[C@@H](F)C6)CC5)nc4c3F)c12] | LCMS m/z [M + 1]: 688.4 ¹HNMR (400 MHz, CD₃OD) δ 8.14 (s, 1H), 7.20-7.23 (m, 1H), 7.03 (t, J = 8.8 Hz, 1H), 5.46 (d, J = 51.6 Hz, 1H), 4.63- 4.80 (m, 2H), 4.37-4.59 (m, 2H), 4.24 (s, 2H), 3.89-3.97 (m, 3H), 3.31-3.62 (m, 5H), 2.38-2.41 (m, 2H), 2.08-2.19 (m, 4H), 0.84-0.96 (m, 4H). |
| 331 | 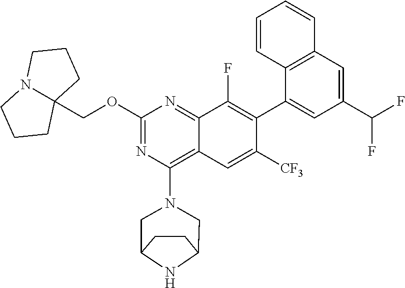 [SMILES: Fc1c(-c2cc(C(F)F)cc3ccccc23)c(C(F)(F)F)cc2c(N3CC4CCC(C3)N4)nc(OCC34CCCN3CCC4)nc12] | LCMS m/z [M + 1]: 642.3 ¹H NMR (400 MHz, CD₃OD) δ 8.28 (d, J = 16.4 Hz, 1H), 8.11 (d, J = 8.4 Hz, 1H), 7.48-7.71 (m, 3H), 7.35-7.44 (m, 1H), 7.00 (t, J = 56 Hz, 1H), 4.78-4.86 (m, 2H), 4.68 (s, 2H), 4.22-4.34 (m, 2H), 3.92-4.09 (m, 2H), 3.61-3.77 (m, 2H), 3.18-3.30 (m, 2H), 1.97-2.39 (m, 12H). |
| 332 | 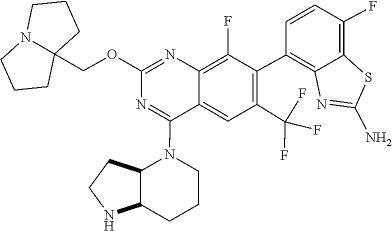 [SMILES: Nc1nc2c(-c3c(C(F)(F)F)cc4c(N5CCC[C@H]6NCC[C@H]65)nc(OCC56CCCN5CCC6)nc4c3F)ccc(F)c2s1] | LCMS m/z [M + 1]: 646 ¹HNMR (400 MHz, CD3OD) 8.23 (s, 1H), 7.25-7.28 (m, 1H),7.04-7.09 (m, 1H), 5.13-5.15 (m, 1H), 4.70-4.71 (m, 1H), 3.90-4.00 (m, 2H), 3.77-3.86 (m, 2H), 3.71-3.77 (m, 4H), 3.50-3.51 (m, 1H), 3.29-3.31 (m, 1H), 2.51-2.55 (m, 2H), 2.01-2.37 (m, 11H), 1.87-1.89 (m, 1H). |
| 333 | 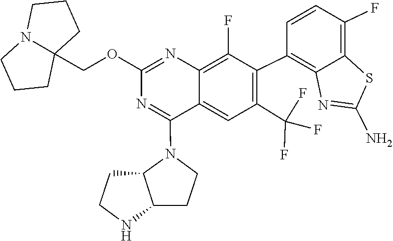 [SMILES: Nc1nc2c(-c3c(C(F)(F)F)cc4c(N5CC[C@@H]6NCC[C@@H]65)nc(OCC56CCCN5CCC6)nc4c3F)ccc(F)c2s1] | LCMS m/z [M + 1]: 632 ¹HNMR (400 MHz, CD₃OD) 8.46-8.48 (m, 1H), 7.23-7.25 (m, 1H), 7.02-7.07 (m, 1H), 4.70 (s, 2H), 4.48-4.58(m, 4H), 3.71-3.73 (m, 1H), 3.70-3.71 (m, 2H), 3.68-3.69 (m, 1H), 3.36-3.50 (m, 2H), 3.27-3.29 (m, 2H), 2.33-2.34 (m, 1H), 2.30-2.31 (m, 2H), 2.10-2.31 (m, 7H). |
| 334 | 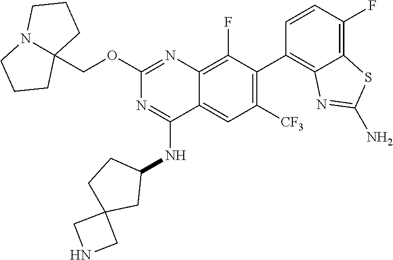 [SMILES: Nc1nc2c(-c3c(C(F)(F)F)cc4c(N[C@@H]5CCC6(CNC6)C5)nc(OCC56CCCN5CCC6)nc4c3F)ccc(F)c2s1] | LCMS m/z [M + 1]: 646 ¹HNMR (400 MHz, CD₃OD) δ 8.62 (s, 1H), 7.22-7.24 (m, 1H), 6.99-7.21 (m, 1H), 4.70-7.84 (m, 5H), 4.00-4.15 (m, 3H), 3.67-3.72 (m, 2H), 3.26-3.28 (m, 3H), 2.62-2.68 (m, 1H), 1.30-2.33 (m, 11H). |
| 335 | 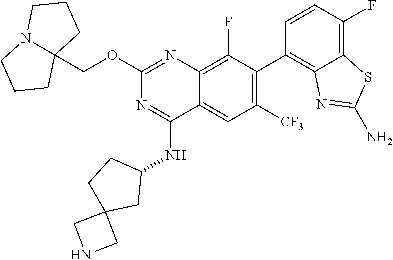 [SMILES: Nc1nc2c(-c3c(C(F)(F)F)cc4c(N[C@H]5CCC6(CNC6)C5)nc(OCC56CCCN5CCC6)nc4c3F)ccc(F)c2s1] | LCMS m/z [M + 1]: 646 ¹HNMR (400 MHz, CD₃OD) δ 8.62 (s, 1H), 7.22-7.24 (m, 1H), 6.99-7.21 (m, 1H), 4.70-7.84 (m, 5H), 4.00-4.15 (m, 3H), 3.67-3.72 (m, 2H), 3.26-3.28 (m, 3H), 2.62-2.68 (m, 1H), 1.30-2.33 (m, 11H). |
| 336 | 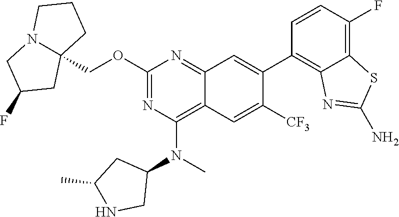 [SMILES: C[C@@H]1C[C@@H](N(C)c2nc(OC[C@@]34CCCN3C[C@H](F)C4)nc3cc(-c4ccc(F)c5sc(N)nc45)c(C(F)(F)F)cc23)CN1] | LCMS m/z [M + 1]: 634.3 ¹HNMR (400 MHz, CD₃OD) δ 8.53 (s, 1H), 7.59 (s, 1H), 7.18-7.22 (m, 1H), 6.97 (t, J = 9.2 Hz, 1H), 5.45-5.64 (m, 2H), 4.66-4.73 (m, 2H), 4.08-4.14 (m, 1H), 3.87-3.99 (m, 4H), 3.55 (s, 3H), 3.45-3.50 (m, 2H), 2.59-2.75 (m, 3H), 2.18-2.46 (m, 5H), 1.50 (d, J = 6.4 Hz, 3H). |
| 337 | 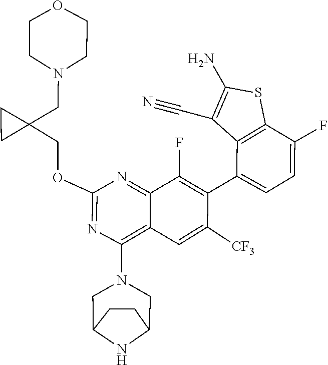 [SMILES: N#Cc1c(N)sc2c(F)ccc(-c3c(C(F)(F)F)cc4c(N5CC6CCC(C5)N6)nc(OCC5(CN6CCOCC6)CC5)nc4c3F)c12] | LCMS m/z [M + 1]: 686.3 ¹HNMR (400 MHz, CD₃OD) δ 8.14 (s, 1H), 7.20-7.23 (m, 1H), 7.03 (t, J = 8.8 Hz, 1H), 4.64-4.73 (m, 2H), 4.54-4.57 (m, 1H), 4.40-4.43 (m, 1H), 4.25 (s, 2H), 4.06-4.07 (m, 2H), 3.75-3.96 (m, 6H), 3.35-3.50 (m, 2H), 3.20-3.21 (m, 2H), 2.03-2.02 (m, 4H), 0.99 (s, 2H), 0.89 (s, 2H). |
| 339 | 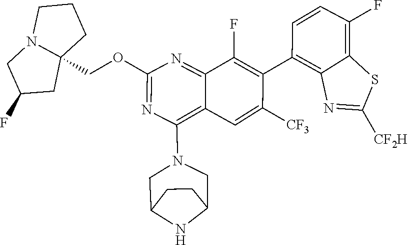 [SMILES: Fc1c(-c2ccc(F)c3sc(C(F)F)nc23)c(C(F)(F)F)cc2c(N3CC4CCC(C3)N4)nc(OC[C@@]34CCCN3C[C@H](F)C4)nc12] | LCMS m/z [M + 1]: 685.2 ¹HNMR (400 MHz, CD₃OD) δ 8.23 (s, 1H), 7.61-7.72 (m, 1H), 7.55 (t, J = 8.8 Hz, 1H), 7.02 (t, J = 56 Hz, 1H), 5.57 (d, J = 52 Hz, 1H), 4.74-4.83 (m, 2H), 4.68 (s, 2H), 4.23-4.27 (m, 2H), 3.79-4.06 (m, 5H), 3.41-3.53 (m, 1H), 2.52-2.79 (m, 2H), 2.27-2.48 (m, 3H), 1.95-2.26 (m, 5H). |
| 340 | 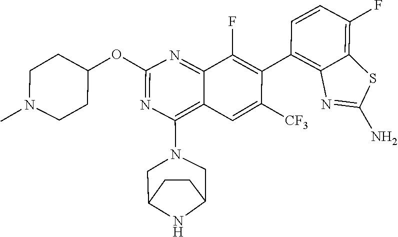 [SMILES: CN1CCC(Oc2nc(N3CC4CCC(C3)N4)c3cc(C(F)(F)F)c(-c4ccc(F)c5sc(N)nc45)c(F)c3n2)CC1] | LCMS m/z [M + 1]: 606.0 ¹HNMR (400 MHz, DMSO-d6) δ 8.2 (s, 1H), 8.06 (s, 1H), 7.92 (s, 2H), 7.17 (t, 1H), 7.04 (t, 1H), 5.07-5.06 (m, 1H), 4.40 (d, 1H), 4.32 (d, 1H), 3.66 (d, 2H), 3.58 (d, 2H), 2.76 (s, 2H), 2.37 (s, 2H), 2.29 (s, 3H), 2.02 (s, 2H), 1.78-1.71 (m, 6H). |
| 341 | 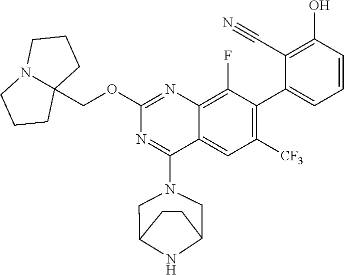 [SMILES: N#Cc1c(O)cccc1-c1c(C(F)(F)F)cc2c(N3CC4CCC(C3)N4)nc(OCC34CCCN3CCC4)nc2c1F] | LCMS m/z [M + 1]: 583.1 ¹H NMR (400 MHz, DMSO-d6) δ 11.67 (s, 1H), 10.35 (s, 1H), 9.45 (d, 1H), 9.25 (s, 1H), 8.19 (s, 1H), 7.64 (t, 1H), 7.19 (d, 1H), 6.92 (d, 1H), 4.69-4.46 (m, 4H), 4.20 (d, 2H), 3.93 (d, 1H), 3.79 (d, 2H), 3.51 (s, 3H), 3.23- 3.22 (m, 2H), 2.20-1.87 (m, 10H) |
| 342 | 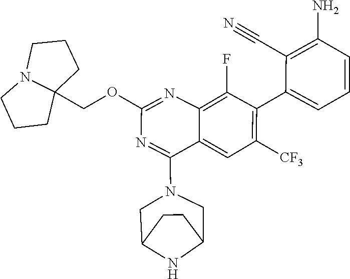 [SMILES: N#Cc1c(N)cccc1-c1c(C(F)(F)F)cc2c(N3CC4CCC(C3)N4)nc(OCC34CCCN3CCC4)nc2c1F] | LCMS m/z [M + 1]: 582.4 ¹HNMR (400 MHz, CD₃OD) δ 8.22 (s, 1H), 7.41-7.43 (m, 1H), 6.96-6.98 (m, 1H), 6.63-6.64 (m, 1H), 4.69-4.73 (m, 3H), 4.26 (s, 2H), 3.97-3.98 (t, 2H), 3.70- 3.72 (m, 2H), 3.30-3.32 (m, 3H), 2.35- 2.37 (m, 2H), 2.10-2.24 (m, 10H). |
| 343 | 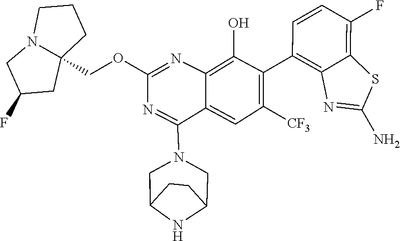 [SMILES: Nc1nc2c(-c3c(C(F)(F)F)cc4c(N5CC6CCC(C5)N6)nc(OC[C@@]56CCCN5C[C@H](F)C6)nc4c3O)ccc(F)c2s1] | LCMS m/z [M + 1]: 648.5 ¹HNMR (400 MHz, CD₃OD) δ 8.12 (s, 1H, 7.26-7.28 (m, 1H), 7.06-7.08 (m, 1H), 5.36-5.38 (m, 1H), 5.25 (s, 0.5H), 5.11 (s, 0.5H), 4.28-4.31 (m, 3H), 3.81-4.04 (m, 6H), 1.92-2.22 (m, 8H), 1.79-1.84 (m, 2H), 1.60-1.66 (m, 2H). |
| 344 | 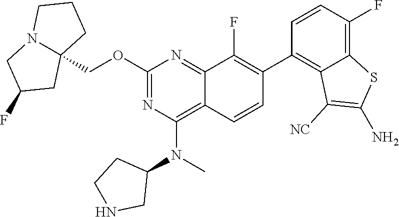 [SMILES: CN(c1nc(OC[C@@]23CCCN2C[C@H](F)C3)nc2c(F)c(-c3ccc(F)c4sc(N)c(C#N)c34)ccc12)[C@@H]1CCNC1] | LCMS m/z [M + 1]: 594.3 ¹HNMR (400 MHz, CD₃OD) δ 8.07 (d, J = 8.8 Hz, 1H), 7.40-7.43 (m, 1H), 7.27- 7.39 (m, 1H), 7.05 (t, J = 8.8 Hz, 1H), 5.56 (d, J = 52 Hz, 1H), 5.17-5.23 (m, 1H), 4.78-4.81 (m, 1H), 4.59-4.66 (m, 1H), 3.83-3.98 (m, 5H), 3.51 (s, 3H), 3.43-3.50 (m, 3H), 2.32-2.62 (m, 8H) |
| 345 | 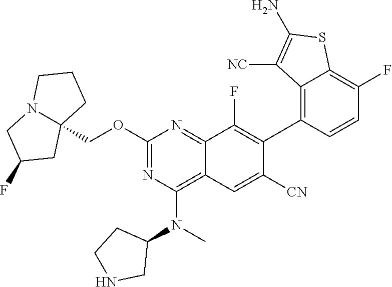 [SMILES: CN(c1nc(OC[C@@]23CCCN2C[C@H](F)C3)nc2c(F)c(-c3ccc(F)c4sc(N)c(C#N)c34)c(C#N)cc12)[C@@H]1CCNC1] | LCMS m/z [M + 1]: 619.0 ¹H NMR (400 MHz, CD₃OD) δ 8.52 (s, 1 H), 7.38-7.35 (m, 1H), 7.12 (t, 1H), 5.64 (s, 0.57 H), 5.51 (s, 0.57 H), 5.39-5.34 (m, 2H), 4.81-4.64 (m, 2H), 3.95-3.86 (m, 3H), 3.69 (t, 1H), 3.55 (s, 3H), 3.51-3.43 (m, 3H), 2.65-2.56 (m, 2H), 2.45-2.31 (m, 1H), 2.40-2.30 (m, 2H), 2.04 (t, 1H), 2.08- 2.01 (m, 1H), 1.62 (t, 1H). |
| 346 | 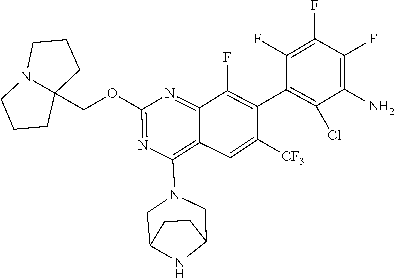 [SMILES: Nc1c(F)c(F)c(F)c(-c2c(C(F)(F)F)cc3c(N4CC5CCC(C4)N5)nc(OCC45CCCN4CCC5)nc3c2F)c1Cl] | LCMS m/z [M + 1]: 645.1 ¹HNMR (400 MHz, CD₃OD) δ 8.78 (s, 1H), 7.34 (dd, 1H), 7.04 (t, 1H), 4.83 (d, 1H), 4.68 (s, 2H), 4.03 (d, 2H), 3.75-3.65 (m, 3H), 3.29-3.21 (m, 1H), 2.38-1.99 (m, 12H). |
| 347 | 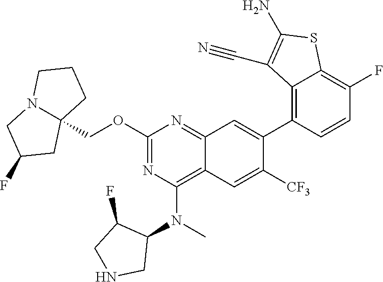 [SMILES: CN(c1nc(OC[C@@]23CCCN2C[C@H](F)C3)nc2cc(-c3ccc(F)c4sc(N)c(C#N)c34)c(C(F)(F)F)cc12)[C@H]1CNC[C@H]1F] | LCMS m/z [M + 1]: 662.3 ¹HNMR (400 MHz, CD₃OD) δ 8.54-8.56 (m, 1H), 7.60 (s, 1H) 7.01-7.22 (m, 1H), 7.00 (t, J = 9.2 Hz, 1H), 5.84 (d, J = 54.4 Hz, 1H), 5.58 (d, J = 52 Hz, 1H), 4.15- 5.25 (m, 1H), 4.61-4.74(m, 2H), 3.65-4.05 (m, 10H), 3.47-3.48 (m, 1H), 2.19-2.64 (m, 6H). |
| 348 | 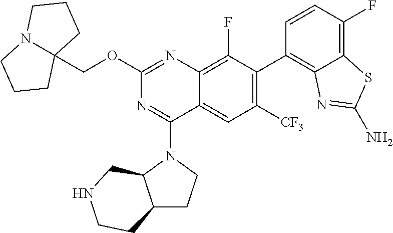 [SMILES: Nc1nc2c(-c3c(C(F)(F)F)cc4c(N5CC[C@H]6CCNC[C@H]65)nc(OCC56CCCN5CCC6)nc4c3F)ccc(F)c2s1] | LCMS m/z [M + 1]: 646.1 ¹H NMR (400 MHz, CD3OD) δ 8.48 (s, 1H), 7.22-7.25 (m, 1H), 7.01-7.06 (m, 1H), 4.68 (m, 2H), 4.15- 4.17 (m, 1H), 3.85-3.88 (m, 1H), 3.64- 3.83 (m, 5H), 3.20-3.29 (m, 3H), 2.73- 2.75 (m, 1H), 2.29-2.36 (m, 2H), 2.04- 2.22 (m, 11H). |
| 349 | 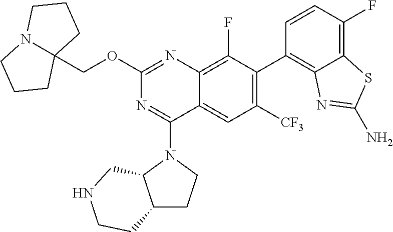 [SMILES: Nc1nc2c(-c3c(C(F)(F)F)cc4c(N5CC[C@@H]6CCNC[C@@H]65)nc(OCC56CCCN5CCC6)nc4c3F)ccc(F)c2s1] | LCMS m/z [M + 1]: 646.1 ¹H NMR (400 MHz, CD3OD) δ 8.49 (s, 1H), 7.21-7.25 (m, 1H), 7.00-7.04 (m, 1H), 4.44-4.69 (m, 2H), 4.20-4.44 (m, 1H), 4.17-4.19 (m, 1H), 3.65-3.73 (m, 4H), 3.32-3.36 (m, 2H), 3.22-3.28 (m, 5H), 2.71-2.74 (m, 1H), 2.02-2.34 (m, 10H). |
| 351 | 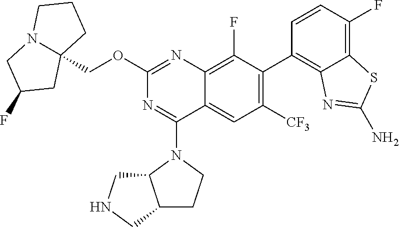 [SMILES: Nc1nc2c(-c3c(C(F)(F)F)cc4c(N5CC[C@@H]6CNC[C@@H]65)nc(OC[C@@]56CCCN5C[C@H](F)C6)nc4c3F)ccc(F)c2s1] | LCMS m/z [M + 1]: 650.1 ¹HNMR (400 MHz, CD₃OD) δ 8.46 (d, 1H), 7.19 (t, 1H), 6.99 (t, 1H), 5.49 (s, 0.5H), 5.36 (s, 0.5H), 5.21 (s, 1H), 4.90 (s, 2H), 4.32-4.49 (m, 3H), 3.79-3.80 (m, 1H), 3.49-3.66 (m, 4H), 3.23-3.27 (m, 4H), 2.15-2.44 (m, 7H). |
| 352 | 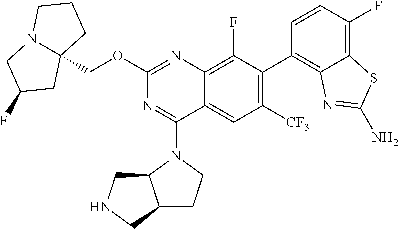 [SMILES: Nc1nc2c(-c3c(C(F)(F)F)cc4c(N5CC[C@H]6CNC[C@H]65)nc(OC[C@@]56CCCN5C[C@H](F)C6)nc4c3F)ccc(F)c2s1] | LCMS m/z [M + 1]: 650.1 ¹H NMR (400 MHz, CD₃OD) δ 8.47 (d, 1H), 7.20 (t, 1H), 6.99 (t, 1H), 5.34 (s, 0.5H), 5.28 (s, 0.5H), 5.20 (s, 1H), 4.90 (s, 2H), 4.32-4.46 (m, 3H), 3.79-3.80 (m, 1H), 3.46-3.63 (m, 4H), 3.25-3.29 (m, 4H), 2.11-2.41 (m, 7H). |
| 353 | 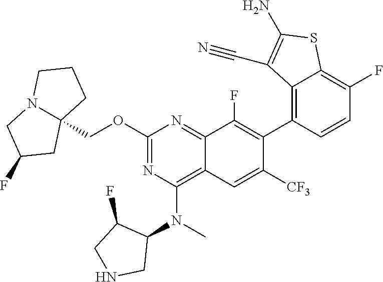 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1CNC[C@H]1F] | LCMS m/z [M + 1]: 680.3 ¹H NMR (400 MHz, CD₃OD) δ 8.41 (s, 1H), 7.22-7.27 (m, 1H), 7.04 (t, J = 8.8 Hz, 1H), 5.84 (d, J = 52 Hz, 1H), 5.56 (d, J = 53.2 Hz, 1H), 5.26-5.35 (m, 1H), 4.62- 4.78 (m, 2H), 3.78-4.06 (m, 7H), 3.67 (s, 3H), 3.42-3.54 (m, 1H), 2.12-2.71 (m, 6H). |
| 354 | 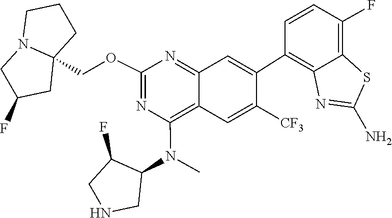 [SMILES: CN(c1nc(OC[C@@]23CCCN2C[C@H](F)C3)nc2cc(-c3ccc(F)c4sc(N)nc34)c(C(F)(F)F)cc12)[C@H]1CNC[C@H]1F] | LCMS m/z [M + 1]: 638.3 ¹HNMR (400 MHz, CD₃OD) δ 8.59 (s, 1H), 7.63 (s, 1H), 7.19-7.27 (m, 1H), 7.00 (t, J = 8.8 Hz, 1H), 5.85 (d, J = 52.6 Hz, 1H), 5.58 (d, J = 51.9 Hz, 1H), 5.25- 5.39 (m, 1H), 4.70 (q, J = 12.0 Hz, 2H), 3.84-4.09 (m, 5H), 3.73-3.82 (m, 2H), 3.68 (s, 3H), 3.42-3.51 (m, 1H), 2.57- 2.78 (m, 2H), 2.28-2.49 (m, 3H), 2.14- 2.25 (m, 1H). |
| 355 | 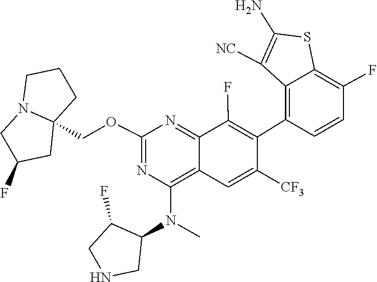 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1CNC[C@@H]1F] | LCMS m/z [M + 1]: 680.2 ¹HNMR(400 MHz, CD₃OD): δ 8.24 (s, 1H), 7.20-7.26 (m, 1H), 7.01-7.07 (m, 1H), 5.88 (d, J = 52.4 Hz, 1H), 5.55 (d, J = 51.2 Hz, 1H), 4.98-5.13 (m, 2H), 4.65- 4.82 (m, 2H), 4.11-4.25 (m, 1H), 3.71- 4.06 (m, 5H), 3.66 (s, 3H), 3.41-3.52 (m, 1H), 2.55-2.72 (m, 2H), 2.27-2.48 (m, 3H), 2.02-2.21 (m, 1H). |
| 356 | 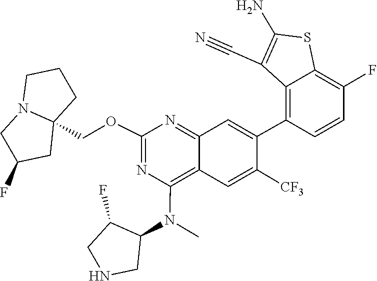 [SMILES: CN(c1nc(OC[C@@]23CCCN2C[C@H](F)C3)nc2cc(-c3ccc(F)c4sc(N)c(C#N)c34)c(C(F)(F)F)cc12)[C@H]1CNC[C@@H]1F] | LCMS m/z [M + 1]: 662.3 ¹HNMR (400 MHz, CD₃OD) δ 8.51 (s, 1H), 7.54 (s, 1H), 7.18 (dd, J = 8.4, 5.6Hz, 1H), 6.98 (t, J = 8.8 Hz, 1H), 5.73 (d, J = 52.8 Hz, 1H), 5.45 (d, J = 53.4 Hz, 1H), 5.01-5.10 (m, 1H), 4.48 (s, 2H), 3.71-3.90 (m, 2H), 3.30-3.67 (m, 8H), 3.22-3.29 (m, 1H), 2.04-2.60 (m, 6H). |
| 357 | 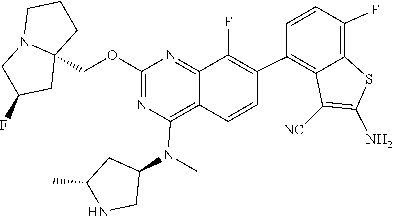 [SMILES: C[C@@H]1C[C@@H](N(C)c2nc(OC[C@@]34CCCN3C[C@H](F)C4)nc3c(F)c(-c4ccc(F)c5sc(N)c(C#N)c45)ccc23)CN1] | LCMS m/z [M + 1]: 608.4 ¹HNMR (400 MHz, CD₃OD) δ 8.08 (d, J = 8.8 Hz, 1H), 7.39-7.42 (m, 1H), 7.27-7.30 (m, 1H), 7.03-7.08 (d, J = 8.8 Hz, 1H), 5.54 (d, J = 51.6 Hz, 1H), 5.38-5.40 (m, 1H), 4.58-4.78 (m, 2H), 5.58-5.69 (m, 1H), 3.87-4.20 (m, 6H), 3.45-3.54 (m, 4H), 2.24-2.62 (m, 8H), 1.66 (s, 3H) |
| 358 | 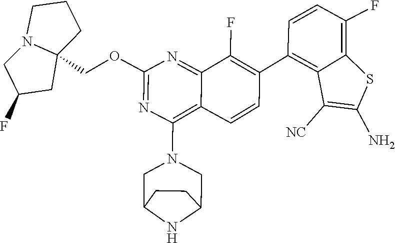 [SMILES: N#Cc1c(N)sc2c(F)ccc(-c3ccc4c(N5CC6CCC(C5)N6)nc(OC[C@@]56CCCN5C[C@H](F)C6)nc4c3F)c12] | LCMS m/z [M + 1]: 606.3 ¹HNMR (400 MHz, CD₃OD) δ 7.91 (d, J = 8.8 Hz, 1H), 7.38-7.45 (m, 1H), 7.28 (dd, J = 7.4, 5.4 Hz, 1H), 7.05 (t, J = 8.8 Hz, 1H), 5.56 (d, J = 53.6 Hz, 1H), 4.60- 4.83 (m, 4H), 4.24 (s, 2H), 3.83-4.09 (m, 5H), 3.44-3.54 (m, 1H), 2.45-2.78 (m, 3H), 2.31-2.42 (m, 2H), 2.09-2.24 (m, 5H). |
| 359 | 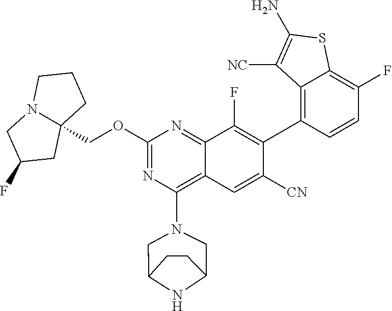 [SMILES: N#Cc1cc2c(N3CC4CCC(C3)N4)nc(OC[C@@]34CCCN3C[C@H](F)C4)nc2c(F)c1-c1ccc(F)c2sc(N)c(C#N)c12] | LCMS m/z [M + 1]: 631.1 ¹HNMR (400 MHz, CDCl₃) δ 8.35 (d, 1H), 7.42-7.43 (m, 2H), 6.93 (t, 1H), 6.70 (s, 2H), 6.40-6.55 (m, 2H), 5.73-5.77 (m, 1H), 5.35-5.36 (m, 0.5H), 5.34-5.35 (m, 0.5H), 5.20-5.26 (m, 1H), 4.47-4.55 (m, 2H), 3.98-4.23 (m, 3H), 3.64-3.72 (m, 4H), 3.34-3.60 (m, 4H), 3.14 (s, 1H), 2.03-2.50 (m, 6H), 1.27 (s, 1H). |
| 360 | 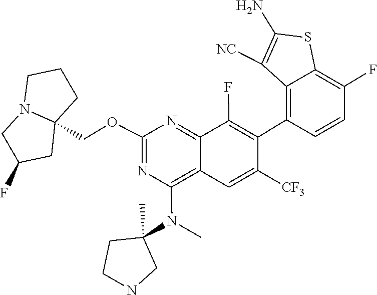 | LCMS m/z [M + 1]: 676.2 ¹H NMR (400 MHz, CD₃OD) δ 8.31 (s, 1H), 7.22 (dd, J = 8.4, 4.8 Hz, 1H), 7.04 (t, J = 8.4 Hz, 1H), 5.56 (d, J = 51.6 Hz, 1H), 4.59-4.80 (m, 2H), 3.80-4.05 (m, 5H), 3.42-3.64 (m, 6H), 2.56-2.76 (m, 3H), 2.31-2.51 (m, 4H), 2.10-2.21 (m, 1H), 1.80 (s, 3H). |
| 361 | 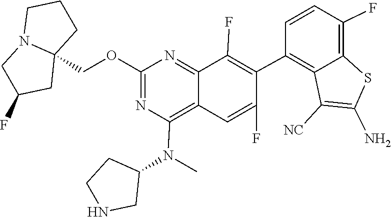 [SMILES: CN(c1nc(OC[C@@]23CCCN2C[C@H](F)C3)nc2c(F)c(-c3ccc(F)c4sc(N)c(C#N)c34)c(F)cc12)[C@H]1CCNC1] | LCMS m/z [M + 1]: 612.4 ¹HNMR (400 MHz, CD₃OD) δ 7.82 (d, J= 9.6 Hz, 1H), 7.28-7.32 (m, 1H), 7.06 (t, J = 8.8 Hz, 1H), 5.53 (d, J = 51.2 Hz, 1H), 5.19-5.23 (m, 1H), 4.64-4.78 (m, 2H), 3.79-3.96 (m, 4H), 3.67-3.69 (m, 1H), 3.40-3.48 (m, 6H), 2.16-2.63 (m, 8H). |
| 362 | 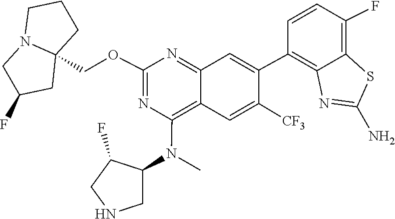 [SMILES: CN(c1nc(OC[C@@]23CCCN2C[C@H](F)C3)nc2cc(-c3ccc(F)c4sc(N)nc34)c(C(F)(F)F)cc12)[C@H]1CNC[C@@H]1F] | LCMS m/z [M + 1]: 638.3 ¹HNMR(400 MHz, CD3OD) δ 8.57 (s, 1H), 7.63 (s, 1H), 7.17-7.22 (m, 1H), 6.93-7.00 (m, 1H), 5.87 (d, J = 58.8 Hz, 1H), 5.58 (d, J = 52.8 Hz, 1H), 4.98-5.12 (m, 1H), 4.68 (s, 2H), 3.66-4.25 (m, 7H). 3.64 (s, 3H), 3.44-3.52 (m, 1H), 2.55-2.75 (m, 2H), 2.30-2.47 (m, 3H), 2.10-2.23 (m, 1H). |
| 363 | 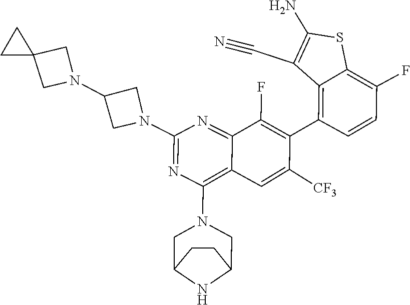 [SMILES: N#Cc1c(N)sc2c(F)ccc(-c3c(C(F)(F)F)cc4c(N5CC6CCC(C5)N6)nc(N5CC(N6CC7(CC7)C6)C5)nc4c3F)c12] | LCMS m/z [M + 1]: 653.3 ¹HNMR (400 MHz, CD₃OD): δ 8.00 (s, 1H), 7.19 (t, J = 8.4 Hz, 1H), 7.01 (t, J = 8.0 Hz, 1H), 4.54-4.68 (m, 5H), 4.21-4.35 (m, 8H), 3.84 (dd, J = 26.0, 14.0 Hz, 2H), 2.12 (s, 4H), 0.86 (s, 4H). |
| 364 | 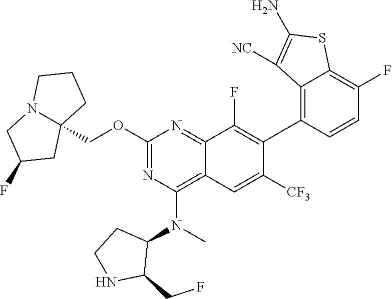 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN[C@@H]1CF] | LCMS m/z [M + 1]: 694.3 ¹HNMR (400 MHz, CD₃OD): δ 8.37 (s, 1H), 7.20-7.23 (m, 1H), 7.01 (t, J = 8.8 Hz, 1H), 5.20-5.42 (m, 2H), 4.96-5.08 (m, 1H), 4.27-4.37 (m, 2H), 3.62 (s, 3H), 3.36-3.44 (m, 5H), 2.98-3.21 (m, 3H), 1.92-2.52 (m, 8H). |
| 365 | 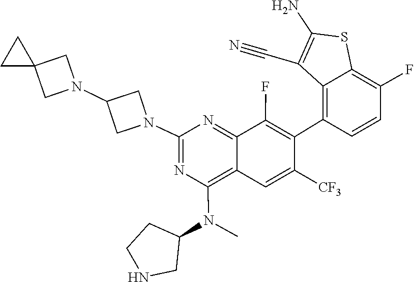 | LCMS m/z [M + 1]: 614.3 ¹HNMR (400 MHz, CD₃OD) δ 8.16 (s, 1H), 7.15-7.19 (m, 1H), 7.01 (t, J = 9.2 Hz, 1H), 5.15-5.22 (m, 1H), 4.65-4.75 (m, 3H), 4.35 (s, 3H), 4.25-4.32 (m, 3H), 3.84 (dd, J = 21.6, 9.6 Hz, 1H), 3.55-3.63 (m, 1H), 3.39-3.45 (m, 5H), 2.34-2.62 (m, 2H), 0.86 (s, 4H). |
| 366 | 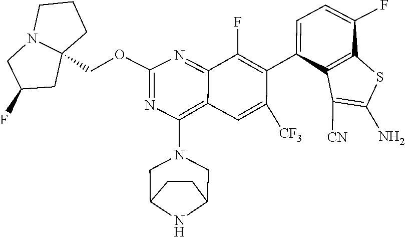 | LCMS m/z [M + 1]: 674.3 ¹HNMR (300 MHz, CD₃OD) δ 8.13 (s, 1H), 7.22 (dd, J = 8.4, 5.4 Hz, 1H), 7.02 (t, J = 9.0 Hz, 1H), 5.36 (d, J = 53.7 Hz, 1H), 4.57 (d, J = 12.6 Hz, 1H), 4.52 (d, J = 11.7 Hz, 1H), 4.32 (d, J = 10.5 Hz, 1H), 4.22 (d, J = 10.5 Hz, 1H), 3.59-3.81 (m, 4H), 3.19-3.30 (m, 2H), 2.91-3.15 (m, 1H), 2.99-3.12 (m, 1H), 2.11-2.42 (m, 3H), 1.93-2.10 (m, 2H), 1.78-1.92 (m, 5H). |
| 367 | 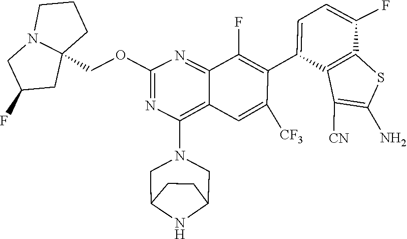 | LCMS m/z [M + 1]: 674.3 ¹HNMR (300 MHz, CD₃OD) δ 8.13 (s, 1H), 7.22 (dd, J = 8.4, 5.7 Hz, 1H), 7.02(t, J = 9.0 Hz, 1H), 5.33(d, J = 53.4 Hz, 1H), 4.60 (d, J = 13.5 Hz, 1H), 4.47 (d, J = 12.0 Hz, 1H), 4.33 (d, J = 10.5 Hz, 1H), 4.25(d, J = 10.8 Hz, 1H), 3.59-3.81 (m, 4H), 3.21- 3.45 (m, 3H), 2.95-3.14 (m, 1H), 2.12- 2.48 (m, 3H), 1.97-2.10 (m, 2H), 1.78- 1.96 (m, 5H). |
| 368 | 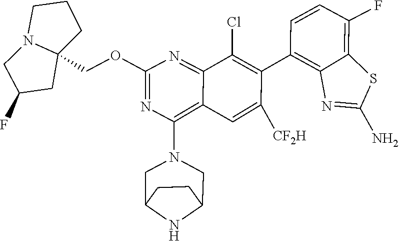 [SMILES: Nc1nc2c(-c3c(C(F)F)cc4c(N5CC6CCC(C5)N6)nc(OC[C@@]56CCCN5C[C@H](F)C6)nc4c3Cl)ccc(F)c2s1] | LCMS m/z [M + 1]: 648.40 ¹H NMR (300 MHz, CD₃OD) δ 8.23 (s, 1H), 7.21 (dd, J = 8.3, 5.5 Hz, 1H), 7.14- 7.06 (m, 1H), 6.59 (d, J = 55.0 Hz, 1H), 5.58 (d, J = 51.9 Hz, 1H), 4.82-4.70 (m, 4H), 4.27 (s, 2H), 4.14-3.91 (m, 5H), 3.49 (s, 1H), 2.80-2.59 (m, 2H), 2.48 (s, 1H), 2.37 (t, J = 8.9 Hz, 2H), 2.14 (s, 5H). |
| 369 | 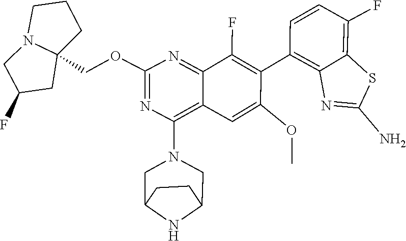 [SMILES: COc1cc2c(N3CC4CCC(C3)N4)nc(OC[C@@]34CCCN3C[C@H](F)C4)nc2c(F)c1-c1ccc(F)c2sc(N)nc12] | LCMS m/z [M + 1]: 612.45 ¹HNMR (300 MHz, CD₃OD) δ 7.30 (dd, J = 8.4, 5.2 Hz, 1H), 7.17 (s, 1H), 7.09 (t, J = 8.8 Hz, 1H), 5.69-5.42 (m, 1H), 4.80-4.61 (m, 4H), 4.26 (s, 2H), 4.11- 3.74 (m, 7H), 3.54-3.40 (m, 1H), 2.81- 2.71 (m, 1H), 2.61-2.25 (m, 5H), 2.16 (s, 5H). |
| 370 | 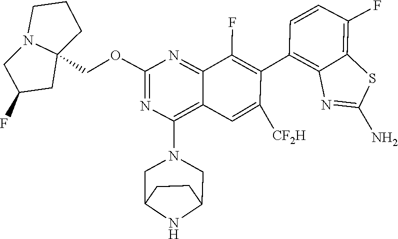 [SMILES: Nc1nc2c(-c3c(C(F)F)cc4c(N5CC6CCC(C5)N6)nc(OC[C@@]56CCCN5C[C@H](F)C6)nc4c3F)ccc(F)c2s1] | LCMS m/z [M + 1]: 632.43 ¹HNMR (300 MHz, CD₃OD) δ 8.10 (s, 1H), 7.27 (dd, J = 8.5, 5.3 Hz, 1H), 7.06 (dd, J = 9.2, 8.5 Hz, 1H), 6.61 (t, J = 55.0 Hz, 1H), 5.57 (d, J = 51.6 Hz, 1H), 4.80- 4.70 (m, 3H), 4.26 (s, 2H), 4.03-3.94 (m, 4H), 3.93-3.82 (m, 2H), 3.50-3.42 (m, 1H), 2.83-2.69 (m, 1H), 2.63-2.55 (m, 1H), 2.48-2.29 (m, 3H), 2.23-2.06 (m, 5H). |
| 371 | 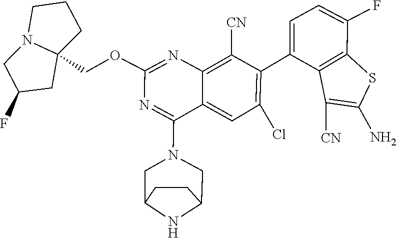 [SMILES: N#Cc1c(-c2ccc(F)c3sc(N)c(C#N)c23)c(Cl)cc2c(N3CC4CCC(C3)N4)nc(OC[C@@]34CCCN3C[C@H](F)C4)nc12] | LCMS m/z [M + 1]: 647.25 ¹HNMR (300 MHz, CD₃OD) δ 8.35 (s, 1H), 7.28 (s, 1H), 7.06 (t, J = 8.8 Hz, 1H), 5.73-5.39 (m, 1H), 4.76-4.70 (m, 3H), 4.28-4.20 (m, 2H), 3.98-3.86 (m, 4H), 3.72-3.63 (m, 2H), 3.57-3.46 (m, 1H), 2.70-2.58 (m, 2H), 2.41-2.29 (m, 3H), 2.16-2.07 (m, 5H). |
| 372 | 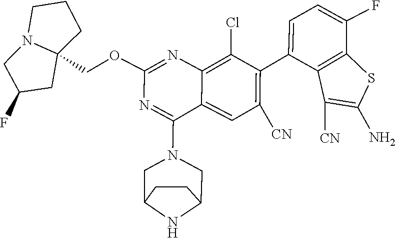 [SMILES: N#Cc1cc2c(N3CC4CCC(C3)N4)nc(OC[C@@]34CCCN3C[C@H](F)C4)nc2c(Cl)c1-c1ccc(F)c2sc(N)c(C#N)c12] | LCMS m/z [M + 1]: 647.24 ¹HNMR (300 MHz, CD₃OD) δ 7.85 (s, 1H), 7.19 (dd, J = 8.4, 5.2 Hz, 1H), 7.00 (t, J = 8.9 Hz, 1H), 5.58 (d, J = 52.0 Hz, 1H), 4.75-4.59 (m, 3H), 4.16 (s, 2H), 4.02-3.71 (m, 5H), 3.50 (s, 2H), 2.79- 2.59 (m, 2H), 2.42 (d, J = 34.2 Hz, 3H), 2.04 (s, 5H). |
| 373 | 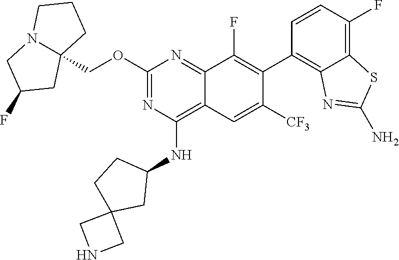 [SMILES: Nc1nc2c(-c3c(C(F)(F)F)cc4c(N[C@@H]5CCC6(CNC6)C5)nc(OC[C@@]56CCCN5C[C@H](F)C6)nc4c3F)ccc(F)c2s1] | LCMS m/z [M + 1]: 646 ¹H NMR (400 MHz, CD₃OD) δ 8.62 (s, 1H), 7.22-7.24 (m, 1H), 6.99-7.21 (m, 1H), 4.70-7.84 (m, 5H), 4.00-4.15 (m, 3H), 3.67-3.72 (m, 2H), 3.26-3.28 (m, 3H), 2.62-2.68 (m, 1H), 1.30-2.33 (m, 11H). |
| 374 | 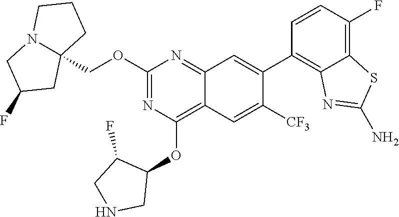 [SMILES: Nc1nc2c(-c3cc4nc(OC[C@@]56CCCN5C[C@H](F)C6)nc(O[C@H]5CNC[C@@H]5F)c4cc3C(F)(F)F)ccc(F)c2s1] | LCMS m/z [M + 1]: 625.2 ¹HNMR (400 MHz, CD₃OD) δ 8.53 (s, 1H), 7.64 (s, 1H), 7.15-7.19 (m, 1H), 6.93 (t, J = 8.8 Hz, 1H), 5.86-5.89 (m, 1H), 5.45 (d, J = 51.2 Hz, 1H), 5.36 (d, J = 52 Hz, 1H), 4.37-4.43 (m, 2H), 3.62-3.66 (m, 1H), 3.36-3.57 (m, 5H), 3.07-3.23 (m, 2H), 1.96-2.44 (m, 6H). |
| 375 | 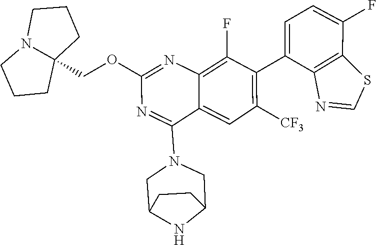 | LCMS m/z [M + 1]: 617.2 ¹HNMR (400 MHz, CD₃OD): δ 9.27 (s, 1H), 8.22 (s, 1H), 7.56 (dd, J = 8.0, 5.2 Hz, 1H), 7.44 (t, J = 8.8 Hz, 1H), 4.75-4.83 (m, 3H), 4.68 (s, 2H), 4.25 (s, 2H), 3.94-4.01 (m, 2H), 3.68 (t, J = 6 Hz, 2H), 3.25-3.29 (m, 1 H), 2.30-2.35 (m, 2 H), 2.10-2.29 (m, 10 H). |
| 376 | 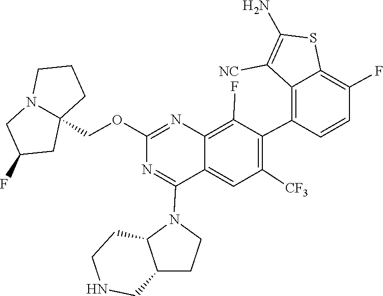 [SMILES: N#Cc1c(N)sc2c(F)ccc(-c3c(C(F)(F)F)cc4c(N5CC[C@@H]6CNCC[C@@H]65)nc(OC[C@@]56CCCN5C[C@H](F)C6)nc4c3F)c12] | LCMS m/z [M + 1]: 688.1 ¹HNMR (400 MHz, CD₃OD) δ 8.53 (s, 1H), 7.23-7.26 (m, 1H), 7.03-7.07 (m, 1H), 5.63 (s, 0.5H), 5.51 (s, 0.5H), 4.50-4.65 (m, 3H), 4.02-4.04 (m, 1H), 3.85-3.87 (m, 3H), 3.42-3.45 (m, 3H), 3.22-3.25 (m, 4H), 2.17-2.81 (m, 10H). |
| 377 | 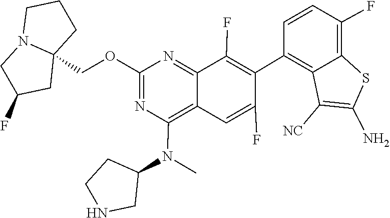 [SMILES: CN(c1nc(OC[C@@]23CCCN2C[C@H](F)C3)nc2c(F)c(-c3ccc(F)c4sc(N)c(C#N)c34)c(F)cc12)[C@@H]1CCNC1] | LCMS m/z [M + 1]: 612.3 ¹HNMR (400 MHz, CD₃OD) δ 7.83 (d, J = 10.0 Hz, 1H), 7.28-7.34 (m, 1H), 7.07 (t, J = 8.8 Hz, 1H), 5.54 (d, J = 51.6 Hz, 1H), 5.16-5.25 (m, 1H), 4.72 (d, J = 12.8 Hz, 1H), 4.66 (d, J = 12.8 Hz, 1H), 3.77- 4.02 (m, 4H), 3.64-3.72 (m, 1H), 3.34- 3.45 (m, 6H), 2.28-2.68 (m, 7H), 2.10- 2.21 (m, 1H). |
| 378 | 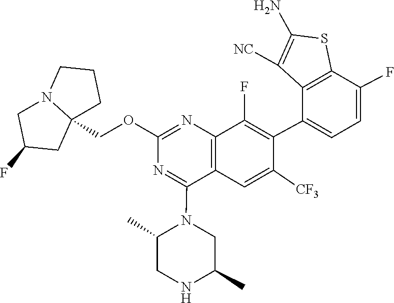 [SMILES: C[C@@H]1CN(c2nc(OC[C@@]34CCCN3C[C@H](F)C4)nc3c(F)c(-c4ccc(F)c5sc(N)c(C#N)c45)c(C(F)(F)F)cc23)[C@@H](C)CN1] | LCMS m/z [M + 1]: 676.3 1H NMR (400 MHz, CD₃OD) δ 8.23 (s, 1 H), 7.23 (dd, J = 7.8, 5.2 Hz, 1H), 7.04 (t, J = 8.8 Hz, 1H), 5.58 (d, J = 52.4 Hz, 1H), 4.66-4.80 (m, 3H), 3.82-4.16 (m, 6H), 3.62-3.68 (m, 1H), 3.43-3.53 (m, 1H), 3.32-3.40 (m, 1H), 2.56-2.79 (m, 2H), 2.30-2.51 (m, 3H), 2.11-2.24 (m, 1H), 1.55- 1.65 (m, 3H), 1.41-1.52 (m, 3H). |
| 379 | 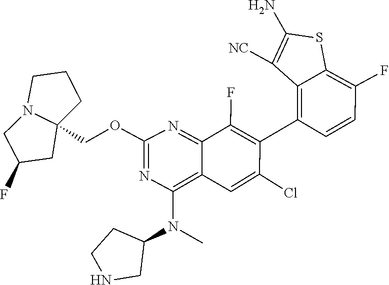 [SMILES: CN(c1nc(OC[C@@]23CCCN2C[C@H](F)C3)nc2c(F)c(-c3ccc(F)c4sc(N)c(C#N)c34)c(Cl)cc12)[C@@H]1CCNC1] | LCMS m/z [M + 1]: 628.3 ¹HNMR (400 MHz, CD₃OD) δ 8.15 (s, 1H), 7.20-7.29 (m, 1H), 7.08 (t, J = 8.8 Hz, 1H), 5.57(d, J = 50.8 Hz, 1H), 5.21- 5.32 (m, 1H), 4.66-4.79 (m, 2H), 3.64- 4.04 (m, 6H), 3.45-3.54 (m, 5H), 2.10- 2.73(m, 8H). |
| 380 | 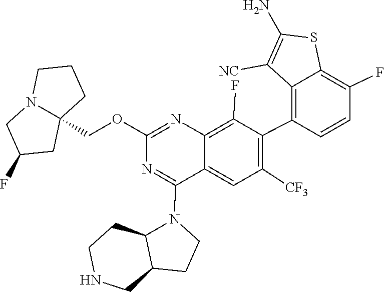 [SMILES: N#Cc1c(N)sc2c(F)ccc(-c3c(C(F)(F)F)cc4c(N5CC[C@H]6CNCC[C@H]65)nc(OC[C@@]56CCCN5C[C@H](F)C6)nc4c3F)c12] | LCMS m/z [M + 1]: 688.1 ¹HNMR (400 MHz, CD₃OD) δ 8.51 (s, 2H), 7.22-7.25 (m, 1H), 7.02-7.07 (m, 1H), 5.56 (s, 0.5H), 5.43 (s, 0.5H), 4.50-4.60 (m, 3H), 4.21 (s, 1H), 3.67-3.74 (m, 3H), 3.47-3.50 (m, 3H), 3.41-3.43 (m, 4H), 2.05-2.59 (m, 10H). |
| 381 | 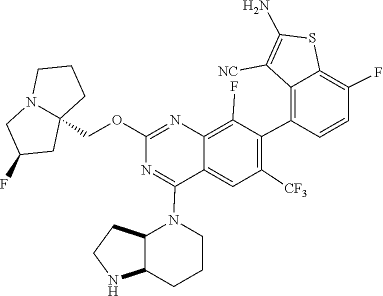 [SMILES: N#Cc1c(N)sc2c(F)ccc(-c3c(C(F)(F)F)cc4c(N5CCC[C@H]6NCC[C@H]65)nc(OC[C@@]56CCCN5C[C@H](F)C6)nc4c3F)c12] | LCMS m/z [M + 1]: 688.1 ¹HNMR (400 MHz, CD₃OD) δ 8.22 (s, 1H), 7.23-7.25 (m, 1H), 7.05 (t, 1H), 5.65 (s, 0.5H), 5.52 (s, 0.5H), 5.17 (d, 1H), 4.65-4.82 (m, 2H), 3.99 (d, 4H), 3.62-3.74 (m, 2H), 3.49-3.54 (m, 2H), 2.43-2.75 (m, 5H), 2.31-2.41 (m, 2H), 1.94-2.25 (m, 6H). |
| 382 | 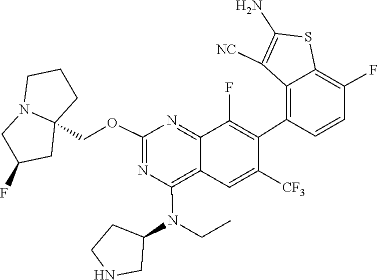 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCNC1] | LCMS m/z [M + 1]: 676.2 |

### Biological Example 1: Determination of Compounds' Binding Affinity to RAS Ligands by SPR

The affinity of compounds of the present disclosure to RAS ligands was assessed using surface plasmon resonance (SPR) binding analysis using a Biacore S200 (GE Healthcare Life Sciences) instrument. The SPR binding analysis employed the following reagents and protocols:

Buffer preparation: To make 0.5 L add required amounts of stocks for: 20 mM Hepes, 150 mM NaCl, 5 mM MgCl₂, 1 mM TCEP, 0.05% P20, and 5 μM nucleotide. Adjust pH to 7.4, then bring volume up to 475 mL 1.05×Filter through 0.2 μm cellulose acetate membrane. Remove a volume of buffer as No DMSO Buffer 1.05× and set aside. Add appropriate volume of DMSO to buffer to a final concentration 5% DMSO.

Chip preparation: Prime 2× with 5% DMSO buffer. Allow system to flow on standby throughout the day.

Sample preparation: Make a 20× compound solution in 100% DMSO of highest compound concentration to be screened (2 mM for final 100 μM compound). Add appropriate volume of 1.05× buffer without DMSO to give 5% DMSO and mix by pipetting. Centrifuge sample 18,000×g 5 min 25° C. Make appropriate serial dilutions from the highest concentration into 5% DMSO running buffer for the concentrations needed. Prepare 5.8% DMSO and 4.5% DMSO solvent correction solutions using buffer without DMSO. Make two more solvent corrections by mixing 200 μL 4.5% 500 μL 5.8%, and 400 μL 4.5% 300 μL 5.8%

Capture Avi-tagged protein: Thaw protein stock on ice, aliquot into single use size and freeze at −80° C. for future use. Dilute protein (avi-KRAS WT, avi-KRAS-G12D, avi-KRAS-G12V, or avi-RhoA) appropriately, to 20 μg/mL to begin in 5% DMSO buffer. Manually inject at 10 μL/min 12 second contact time. Adjust flow and or contact time to achieve desired level of captured protein. When desired level of protein is captured, flow for 10 minutes to stabilize signal.

General Binding Level Screen Method: Choose LMW screen template in Fragment/LMW subfolder in Binding screen folder. 30 μL/min high performance injection, 60 sec contact and dissociation time, carry over control, extra wash with 50% DMSO for each injection. Fifteen Startup cycles to condition. Control samples before and after compound samples. For GppNHp loaded KRAS, control is 20 μM Rafl RBD domain. Adjust this sample to final 5% DMSO to match running buffer. This process is performed at a temperature of 25° C. using a single chip per experiment.

Binding affinity determination: Compounds selected from the binding level screen are evaluated in a dose response to determine the binding affinity (K_{D}). Serial dilutions of compounds are prepared and injected over the sensor chip using a flow rate of 30 μL/min with a 60 second contact and dissociation time. After double referencing of the sensorgrams the total level of compound binding at each concentration is calculated. The steady state binding affinity is calculated with a Langmuir binding model assuming a 1:1 interaction with a constrained Rmax using the BIAevaluation software.

### Biological Example 2: Disrupting RAS-Effector Binding HTRF Assay

A protein:protein interaction (PPI) Homogeneous Time Resolved Fluorescence (HTRF) assay was used to determine the effectiveness of compounds of the present disclosure in disrupting KRAS protein and effector (RAF1 or PIK3CA) binding.

The HTRF assay used the following reagents and proteins: 375 nM Avi-KRAS G12D/Q25A (1-169) GppNHp/3×FLAG-PI3KCA (157-299); 100 nM Avi-KRAS G12D (1-169) GppNHp/RAF1 RBD-3×FLAG (52-151); 35 nM Avi-PI3K RBD CA-3×FLAG; Assay Buffer: 50 mM Tris pH 7.5, 100 mM NaCl, 5 mM MgCl₂, 0.1% BSA, 0.01% Tween, 1 mM TCEP, 10% DMSO; Bead Buffer: 50 mM Tris pH 7.5, 0.01% Tween 20; Assay volume: 20 μL (384 well plate-low volume format); and Compound titration: 100-0.02 μM, 3× dilution series.

The HTRF assay employed the following protocol:

Compounds were dispensed in assay plate (384-well, Grenier Bione #784075) using Echo (model 555) with dose response settings: 200 nL final volume, titration from 100 μM as a 10-point dilution series. Protein mix was prepared in assay buffer, and dispensed on plates, 10 μL per well, then incubated for 1 h at room temperature, with 700 rpm shaking. Reagents mix was prepared and dispensed on plates, 10 μL per well, then incubated for 1 h at room temperature, with 700 rpm shaking.

Plates were analyzed on an Envision plate reader using the following setting: Excitation 320 nM, Bandwidth 75 nM; Emission 615 nM, Bandwidth 85 nM, Gain 100%, Flashes 100, Lag 60 μs. Data was reported as percentage of activity, with DMSO as 100%. Data was plotted and analyzed using Prism 8. The SPR Kd, biochemical Rafl-KRAS G12D-GppNHP and PI3KCA-KRAS G12D/Q25A-GppNHp disruption assay IC₅₀ of selected compounds described herein are shown in Table 10.

### Biological Example 3. Cell-Based pERK HTRF Assay

pERK assay (Perkin Elmer) was used to determine the effectiveness of compounds in disrupting KRAS G12D protein/effector signaling in cells.

On Day 1, cells (GP2d) were seeded into 96-well plates at 3×10⁴ cells/well in 80 μl complete growth media (DMEM, 10% FBS).

On Day 2, cells were treated with compounds at 0.25% DMSO. The source plate was created with compounds diluted in media at 5-fold the final assay concentration. The compounds were run in a 9-point concentration curve starting at 1 μM, with a half-log dilution between concentrations. 20 μL was transferred onto the cell plates (final volume in wells was 100 μL). Plates were harvested after 60 min incubation by aspirating media and adding kit-supplied 1× supplemented lysis buffer to all wells (50 μl per well). Plates were then placed on a plate shaker and incubated at 850 rpm for an additional 30 min.

Antibody mixture solution is prepared by diluting aliquoted d2 and Eu Cryptate antibodies 1:20 in kit supplied detection buffer, mix the diluted antibodies solutions (1:1 v:v). Four μL of this solution is then added to a 384-well detection plate (Perkin Elmer; 6008230).

Samples were homogenized by pipetting up and down and then transferred (16 μL of cell lysates) from the 96-well cell culture plate to two wells of the HTRF 384-well detection plate containing the antibody solution. Plates were centrifuged (524 g for 1 min) and allowed to incubate between 4 and 24 h at room temperature. Maximum signal is reached after 4 h incubation time and remains stable over a period of 24 hours. Therefore, readings can be made between 4 and 24 h of incubation. Plates were centrifuged again (524 g for 1 min), then analyzed on the EnVision plate reader using the following settings: Excitation 320 nm, Bandwidth 75 nm; Emission 615 nm, Bandwidth 85 nm; Gain 100%; Flashes 100; Lag 60 μs.

Table 10 includes biological characterization data for selected compounds using the assays described in Biological Examples 1-3. For KRAS G12D binding affinity: A: K_{D}≤0.1 μM; B: 0.1 μM<K_{D}≤1 μM; C: 1 μM<K_{D}≤10 μM; D: 10 μM<K_{D}≤30 μM. For Rafl-KRAS G12D-GppNHP and PI3KCA-KRAS G12D/Q25A-GppNHp disruption assay: A: IC₅₀≤0.1 μM; B: 0.1 μM<IC₅₀≤1 μM; C: 1 μM<IC₅₀≤10 μM; D: 10 μM<IC₅₀≤30 μM. For GP2d pERK HTRF assay: A: IC₅₀≤0.1 μM; B: 0.1 μM<IC₅₀≤1 μM; C: IC₅₀>1 μM. Blanks in the table represent that a compound was not tested in the indicated assay.

TABLE 10
Biological characterization of selected compounds of the present disclosure.

| | | | | PI3KCA- | |
|---|---|---|---|---|---|
| | GDP KRAS | GppNHp | Raf1-GppNHp- | GppNHp-KRAS | |
| Compound | G12D binding | KRAS G12D | KRAS G12D | G12D/Q25A | pERK-Gp2D- |
| No. | affinity | binding affinity | disruption | disruption | 1hr |
| 1 | C | D | D | D | |
| 5 | C | D | D | D | |
| 46 | C | D | D | D | |
| 47 | C | D | D | D | |
| 49 | A | C | C | C | |
| 53 | A | C | D | D | |
| 54 | B | C | C | C | |
| 55 | A | C | C | C | |
| 56 | B | D | D | D | |
| 59 | | C | D | D | |
| 60 | | D | D | D | |
| 61 | | C | C | C | |
| 62 | | D | D | D | |
| 63 | | D | D | D | |
| 64 | | | D | D | |
| 65 | | | D | D | |
| 66 | | | D | D | |
| 67 | | | D | D | |
| 68 | | | D | D | |
| 69 | | | D | D | |
| 70 | | | C | D | |
| 71 | | | D | D | |
| 73 | | | C | D | |
| 74 | | D | D | D | |
| 75 | | | C | D | |
| 76 | | | D | D | |
| 77 | | B | C | C | |
| 78 | | | D | D | |
| 80 | | | D | D | |
| 81 | | | D | D | |
| 82 | | | C | C | |
| 83 | | | C | C | |
| 84 | | | D | D | |
| 85 | | | D | D | |
| 86 | | | D | D | |
| 87 | | | D | D | |
| 88 | | | D | D | |
| 89 | | B | C | C | |
| 90 | | | D | D | |
| 91 | | | C | C | |
| 92 | | | D | D | |
| 93 | | | D | D | |
| 94 | | | D | D | |
| 95 | | | D | D | |
| 96 | | | D | D | |
| 97 | | | C | D | |
| 98 | | | C | C | |
| 99 | | | C | D | |
| 100 | | | D | D | |
| 101 | | | D | D | |
| 102 | | | D | D | |
| 103 | | | D | D | |
| 104 | | | D | D | |
| 105 | | | D | D | |
| 106 | | | C | C | |
| 107 | | | D | D | |
| 108 | | | D | D | |
| 109 | | | D | D | |
| 112 | | | C | C | |
| 113 | | | B | C | |
| 114 | | | C | C | |
| 115 | | | C | C | |
| 116 | | | C | C | |
| 119 | | | C | C | |
| 120 | A | A | B | B | C |
| 122 | | | C | C | |
| 123 | | | C | C | |
| 124 | | | D | D | |
| 125 | | | D | D | |
| 126 | | | C | C | C |
| 127 | | | B | C | C |
| 128 | | | D | D | |
| 129 | | | B | C | C |
| 130 | | | C | C | |
| 131 | | | D | D | |
| 132 | | | C | C | |
| 133 | | | B | C | C |
| 134 | | | C | D | |
| 135 | | | C | C | |
| 136 | | | D | D | |
| 137 | | | C | D | |
| 140 | | | C | D | |
| 141 | | | D | D | |
| 143 | | | D | D | |
| 144 | | | D | D | |
| 145 | | | D | D | |
| 146 | | | C | D | |
| 147 | | | D | D | |
| 148 | | | C | C | |
| 149 | | | C | C | |
| 150 | | | D | D | |
| 151 | | | C | D | |
| 152 | | | D | D | |
| 153 | | | C | D | |
| 154 | | | D | D | |
| 155 | | | D | C | |
| 156 | | | D | D | |
| 157 | A | A | B | B | B |
| 158 | A | A | B | B | A |
| 159 | | | B | D | |
| 160 | | | D | D | |
| 161 | | | C | C | |
| 162 | | | D | D | |
| 163 | | | D | D | |
| 164 | | | D | D | |
| 165 | | | D | D | |
| 166 | | | D | D | |
| 167 | | | D | | |
| 168 | | | D | | |
| 169 | | | D | | |
| 170 | | | D | D | |
| 171 | | | C | | |
| 172 | | | D | | |
| 173 | | | C | | |
| 174 | | | C | | C |
| 175 | | | D | C | |
| 176 | | | D | D | |
| 177 | | | D | D | |
| 178 | | | D | D | |
| 179 | | | C | D | |
| 180 | | | D | D | |
| 181 | | | C | C | |
| 182 | | | C | D | |
| 183 | | | D | D | |
| 184 | | | B | C | C |
| 185 | | | B | B | B |
| 186 | | | D | D | |
| 187 | | | B | C | C |
| 188 | | | B | C | C |
| 189 | | | C | C | |
| 190 | | | C | C | |
| 191 | | | C | C | |
| 192 | | | C | C | |
| 193 | | | D | | |
| 194 | | | C | | |
| 196 | | | D | D | |
| 197 | | | D | D | |
| 198 | | | C | C | |
| 199 | | | B | C | C |
| 201 | | | D | D | |
| 202 | | | B | C | |
| 203 | | | D | D | |
| 204 | | | D | D | |
| 205 | | | D | D | |
| 206 | | | B | C | |
| 207 | | | B | C | A |
| 208 | | | D | D | |
| 209 | | | B | C | |
| 210 | | | C | D | |
| 211 | | | C | D | |
| 212 | | | B | C | |
| 214 | | | B | D | |
| 215 | | | C | D | |
| 216 | | | C | C | |
| 217 | | | C | C | |
| 219 | A | A | A | B | A |
| 220 | | | B | B | A |
| 221 | A | C | C | C | B |
| 222 | | | C | D | |
| 223 | | | B | B | A |
| 224 | | | D | D | |
| 225 | | | C | D | |
| 226 | | | B | B | A |
| 227 | | | C | C | |
| 228 | | | C | C | |
| 229 | | | D | D | |
| 230 | | | C | C | |
| 231 | | | C | C | |
| 232 | | | B | D | A |
| 233 | | | C | C | |
| 234 | | | C | C | |
| 235 | | | C | D | |
| 236 | | | B | B | C |
| 237 | | | B | C | |
| 238 | | | C | C | |
| 239 | | | C | C | |
| 240 | | | C | C | |
| 241 | A | A | B | B | A |
| 242 | | | D | D | |
| 243 | | | D | D | |
| 245 | A | A | A | A | A |
| 246 | A | C | C | C | |
| 247 | | | B | B | C |
| 248 | A | A | A | B | C |
| 249 | A | C | C | D | |
| 250 | | | B | B | |
| 251 | | | B | C | A |
| 252 | | | B | B | A |
| 253 | | | C | C | |
| 255 | A | A | A | B | A |
| 256 | | | B | B | A |
| 258 | | | B | B | A |
| 260 | A | A | A | B | A |
| 261 | | | B | B | A |
| 263 | | | C | C | |
| 264 | | | B | B | C |
| 265 | | | B | C | |
| 266 | | | B | C | |
| 268 | | | C | C | |
| 270 | | | C | D | |
| 271 | | | B | C | |
| 272 | | | B | C | |
| 273 | | | C | C | |
| 274 | | | B | B | B |
| 275 | | | C | C | A |
| 276 | | | B | C | C |
| 277 | | | C | C | |
| 278 | | | C | C | |
| 279 | | | D | C | |
| 280 | | | C | C | |
| 281 | | | D | D | |
| 282 | | | C | C | |
| 283 | | | B | B | B |
| 284 | | | B | B | |
| 285 | | | B | B | A |
| 286 | | | B | B | |
| 287 | | | C | C | |
| 288 | | | B | C | |
| 289 | A | B | B | B | |
| 290 | A | B | B | B | A |
| 29 | C | D | D | D | |
| 292 | | | C | C | |
| 293 | | | B | B | B |
| 294 | | | B | C | |
| 295 | | | C | C | |
| 296 | | | B | C | A |
| 297 | | | B | C | B |
| 299 | | | B | B | A |
| 300 | | | B | B | B |
| 301 | A | A | B | B | A |
| 302 | | | C | C | |
| 304 | | | C | C | C |
| 307 | | | B | C | |
| 308 | | | B | C | |
| 309 | | | C | C | B |
| 311 | | | B | D | A |
| 312 | | | C | D | B |
| 313 | | | B | C | A |
| 314 | | | B | C | B |
| 315 | | | C | D | B |
| 316 | | | C | C | B |
| 318 | | | C | D | B |
| 319 | | | B | B | A |
| 320 | | | B | B | A |
| 322 | | | B | C | B |
| 323 | | | A | B | A |
| 324 | | | C | C | B |
| 325 | | | C | C | C |
| 326 | | | C | C | C |
| 327 | | | B | C | C |
| 328 | | | C | C | C |
| 329 | | | D | D | C |
| 330 | | | A | B | A |
| 331 | | | D | D | C |
| 332 | | | B | C | B |
| 333 | | | C | C | C |
| 334 | | | C | C | C |
| 335 | | | B | B | C |
| 336 | | | B | C | B |
| 337 | | | A | B | A |
| 339 | | | D | D | C |
| 340 | | | C | C | B |
| 341 | | | D | D | C |
| 342 | | | D | D | C |
| 343 | | | D | D | C |
| 344 | | | B | B | B |
| 345 | | | B | B | B |
| 346 | | | D | D | C |
| 347 | | | B | B | A |
| 348 | | | C | C | C |
| 349 | | | C | C | C |
| 350 | | | B | B | A |
| 351 | | | B | B | B |
| 352 | | | C | C | B |
| 353 | | | B | B | A |
| 354 | | | B | B | B |
| 355 | | | B | C | A |
| 356 | | | C | C | B |
| 357 | | | C | C | B |
| 358 | | | B | C | A |
| 359 | | | B | B | A |
| 360 | | | A | B | A |
| 361 | | | B | B | B |
| 362 | | | D | D | |
| 363 | | | B | B | A |
| 364 | | | A | B | A |
| 365 | | | B | B | B |
| 366 | | | A | A | A |
| 367 | | | C | C | B |
| 368 | | | B | B | A |
| 369 | | | B | C | |
| 370 | | | B | B | A |
| 371 | | | B | B | A |
| 372 | | | C | C | B |
| 373 | | | C | C | C |
| 374 | | | C | D | C |
| 375 | | | D | D | |
| 376 | | | B | B | A |
| 377 | | | B | B | B |
| 378 | | | A | B | A |
| 379 | | | B | B | A |

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations, and equivalents. It is intended that the following claims define the scope of the invention and that methods and structures within the scope of these claims and their equivalents be covered thereby.

1. A compound represented by Formula I: 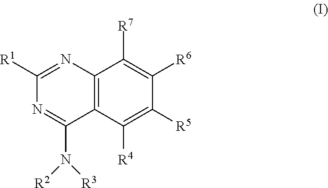
or a pharmaceutically acceptable salt thereof, wherein:
R¹ is selected from —OR⁸, a 4-6 membered heterocycle comprising a nitrogen atom, and H, wherein the heterocycle is unsubstituted or substituted with one or more R¹⁶;
R² is selected from H, a 3-6 membered carbocycle, and C₁₋₆ alkyl, wherein the 3-6 membered carbocycle or the C₁₋₆ alkyl is unsubstituted or substituted with one or more R¹³;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁵ is selected from H, halogen, —OR¹², —CN, C₁₋₆alkyl, C₂₋₆alkenyl, C₂₋₆alkynyl, a 3-6 membered carbocycle, a 3-6 membered heterocycle, a 5-6 membered heteroaryl, and phenyl, wherein any C₁₋₆alkyl is unsubstituted or substituted with one or more R¹³, and wherein any carbocycle, heterocycle, heteroaryl, and phenyl is unsubstituted or substituted with one or more R¹⁴;
R⁶ is a phenyl, monocyclic heteroaryl, bicyclic aryl, or bicyclic heteroaryl, wherein any phenyl, monocyclic heteroaryl, bicyclic aryl, and bicyclic heteroaryl is unsubstituted or substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶, and wherein an alkyl moiety of any alkylheterocycle is selected from C₁₋₆ alkyl;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from halogen, —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, —OR¹², —CN, —N(R¹²)₂, and C₁₋₆alkyl, wherein any C₁₋₆alkyl is unsubstituted or substituted with one or more R¹³;
each R¹⁵ is independently selected from halogen, —OR¹², —CN, —N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, —OR¹², and 3-6 membered heterocycle, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl, 3-6 membered heterocycle, and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, halogen, and 3-6 membered carbocycle;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

2. The compound of claim 1, wherein R¹ is —OR⁸.

3. The compound of claim 1 or 2, wherein R⁸ is a heterocycle, wherein the heterocycle is unsubstituted or substituted with one or more R¹⁶.

4. The compound of any one of claims 1-3, wherein R⁸ is a 4-8 membered heterocycle having 1-2 heteroatoms independently selected from nitrogen, oxygen, and sulfur, wherein the heterocycle is unsubstituted or substituted with one or more R¹⁶.

5. The compound of claim 1 or 2, wherein R⁸ is an alkylheterocycle, wherein the alkylheterocycle is unsubstituted or substituted with one or more R¹⁶.

6. The compound of claim 5, wherein the alkylheterocycle is —CH₂(heterocycle), wherein the heterocycle is unsubstituted or substituted with one or more R¹⁶.

7. The compound of claim 5 or 6, wherein the heterocycle of the alkylheterocycle is a 4-8 membered heterocycle having 1-2 heteroatoms independently selected from nitrogen, oxygen, and sulfur, and the heterocycle is unsubstituted or substituted with one or more R¹⁶.

8. The compound of any one of claims 1-7, wherein R⁸ is substituted with one or more R¹⁶.

9. The compound of any one of claims 1-8, wherein each R¹⁶ is independently selected from halogen, C₁₋₆alkyl, and —OR¹², wherein any C₁₋₆alkyl is unsubstituted or substituted with one or more R¹³.

10. The compound of any one of claims 1, 2, and 5-9, wherein —OR⁸ is: 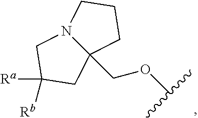
wherein R^{a} and R^{b} are each independently selected from halogen, C₁₋₆alkyl, —OR, and H, wherein any C₁₋₆alkyl is unsubstituted or is substituted with one or more R¹³.

11. The compound of any one of claims 1-9, wherein —OR⁸ is selected from: 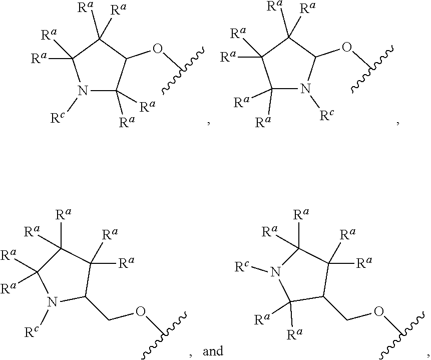
wherein:
each R^{a} is independently selected from halogen, C₁₋₆ alkyl, —OR¹², and H; and
R^{c} is selected from C₁₋₆ alkyl,
wherein a C₁₋₆ alkyl of a R^{a} or R^{c} is unsubstituted or is substituted with one or more R¹³.

12. The compound of claim 11, wherein one R^{a} is selected from halogen, C₁₋₆ alkyl, and —OR¹², and the other R^{a}'s are H.

13. The compound of claim 1, wherein —OR⁸ is selected from:

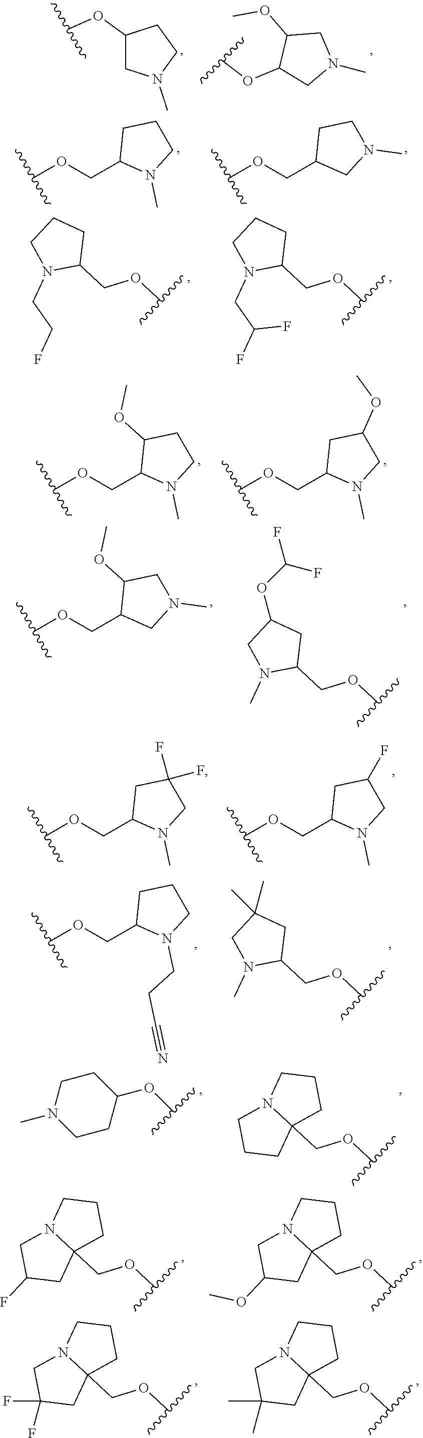

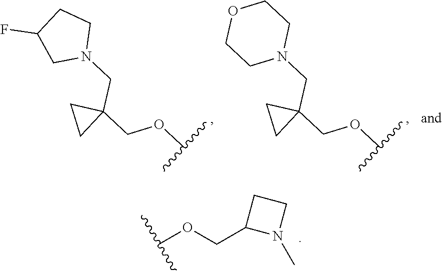

14. The compound of claim 1, wherein R¹ is a 4-6 membered heterocycle comprising a nitrogen atom, wherein the heterocycle is unsubstituted or substituted with one or more R¹⁶.

15. The compound of claim 1 or 14, wherein each R¹⁶ is independently selected from —N(R¹²)₂, C₁₋₆alkyl, and 3-6 membered heterocycle.

16. The compound of any one of claim 1, 14, and 15, wherein R¹ is selected from:

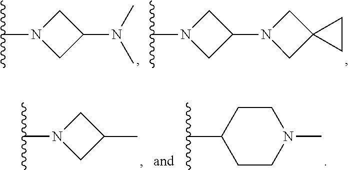

17. The compound of claim 1, wherein R¹ is H.

18. The compound of any one of claims 1-17, wherein R² is H.

19. The compound of any one of claims 1-17, wherein R² is C₁₋₆ alkyl, unsubstituted or substituted with one or more R¹³.

20. The compound of any one of claims 1-17, wherein R² is a 3-6 membered carbocycle.

21. The compound of any one of claim 1-20, wherein R³ is C₁₋₆alkyl substituted with one or more R⁹.

22. The compound of claim 21, wherein R⁹ is —N(R¹⁷)₂.

23. The compound of claim 1 or 21, wherein R³ is selected from:

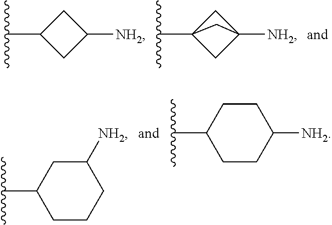

24. The compound of any one of claims 1-20, wherein R³ is a carbocycle, unsubstituted or substituted with one or more R¹⁰.

25. The compound of claim 24, wherein R³ is a 4-6 membered carbocycle, unsubstituted or substituted with one or more R¹⁰.

26. The compound of claim 24 or 25, wherein R¹⁰ is —N(R¹⁹)₂, wherein each R¹⁹ is independently selected from unsubstituted or substituted C₁₋₆ alkyl and H.

27. The compound of any one of claims 1, 25, and 26, wherein R³ is selected from:

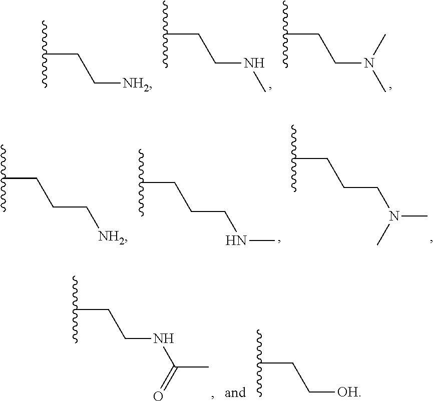

28. The compound of any one of claims 1-20, wherein R³ is a heterocycle, unsubstituted or substituted with one or more R¹⁰.

29. The compound of claim 28, wherein R³ is a 4-8 membered heterocycle having 1-2 heteroatoms independently selected from nitrogen, oxygen, and sulfur, wherein the heterocycle is unsubstituted or substituted with one or more R¹⁰.

30. The compound of claim 28 or 29, wherein each R¹⁰ is independently selected from halogen, —N(R¹⁹)₂, —C(O)R¹⁹, —C(O)N(R¹⁹)₂, —C(O)(C₁₋₆alkyl)N(R¹⁹)₂, and C₁₋₆alkyl, wherein any C₁₋₆alkyl is unsubstituted or substituted with one or more R²⁰.

31. The compound of any one of claims 1 and 28-30, wherein R³ is selected from:

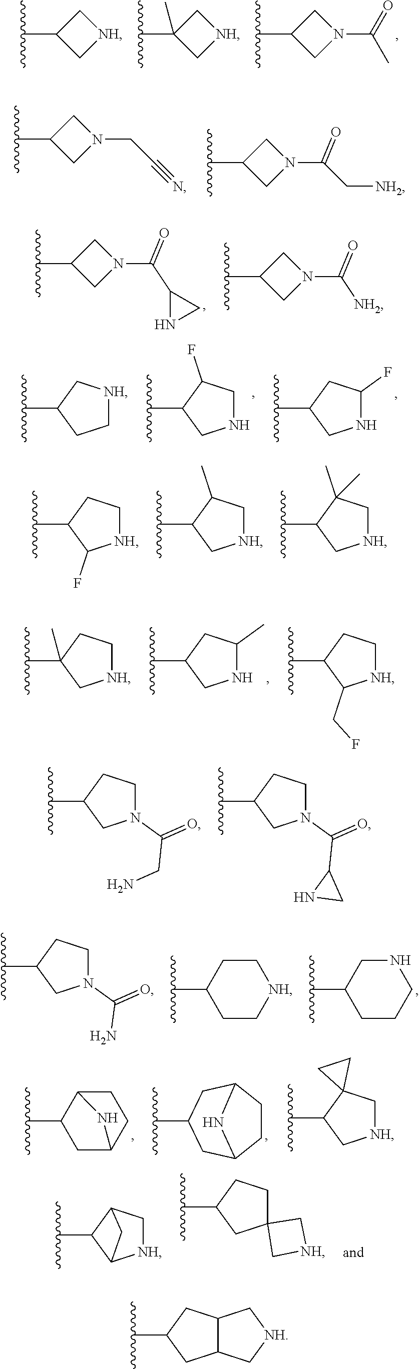

32. The compound of any one of claims 1-20, wherein R² and R³, together with the atom to which they are attached, form a heterocycle that is unsubstituted or substituted with one or more R¹¹.

33. The compound of claim 32, wherein R² and R³, together with the atom to which they are attached, form a 4-9 membered heterocycle having 0-2 additional heteroatoms independently selected from nitrogen, oxygen, and sulfur, wherein the heterocycle is unsubstituted or substituted with one or more R¹¹.

34. The compound of any one of claims 1, 32, and 33, wherein each R¹¹ is independently selected from —N(R¹⁹)₂, —C(O)R¹⁹, —C(O)N(R¹⁹)₂, —C(O)(C₁₋₆alkyl)N(R¹⁹)₂, and C₁₋₆alkyl, wherein any C₁₋₆alkyl is unsubstituted or substituted with one or more R²⁰.

35. The compound of any one of claims 1, 32, and 33, wherein R² and R³, together with the atom to which they are attached, form a heterocycle selected from:

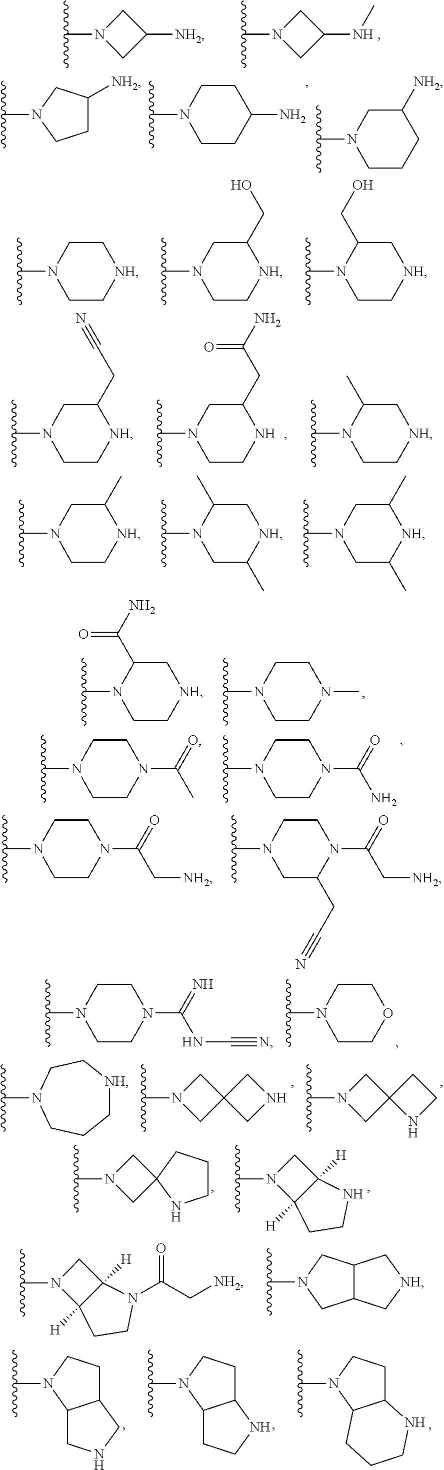

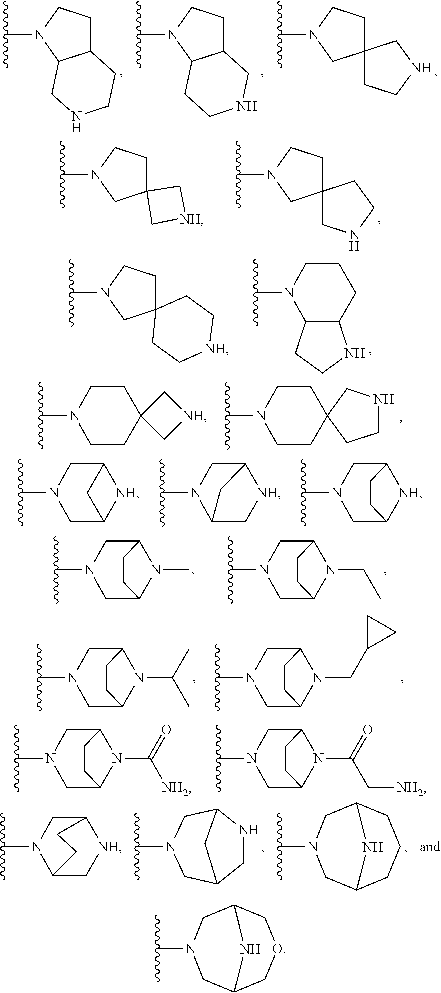

36. The compound of any one of claims 1-35, wherein R⁶ is a bicyclic aryl or bicyclic heteroaryl, wherein the aryl or heteroaryl are unsubstituted or substituted with one or more R¹⁵.

37. The compound of any one of claims 1-36, wherein R⁶ is a 9-10 membered heteroaryl having 1-3 heteroatoms independently selected from nitrogen, oxygen, and sulfur that is unsubstituted or substituted with one or more R¹⁵.

38. The compound of claim 37, wherein R⁶ is substituted with one or more R¹⁵.

39. The compound of claim 38, wherein each R¹⁵ is independently selected from halogen, —CN, and —N(R¹²)₂.

40. The compound of claim 38 or 39, wherein at least one R¹⁵ is —N(R¹²)₂.

41. The compound of any one of claims 38-40, wherein at least one R¹⁵ is a halogen.

42. The compound of any one of claims 1, 36, and 37, wherein R⁶ is selected from:

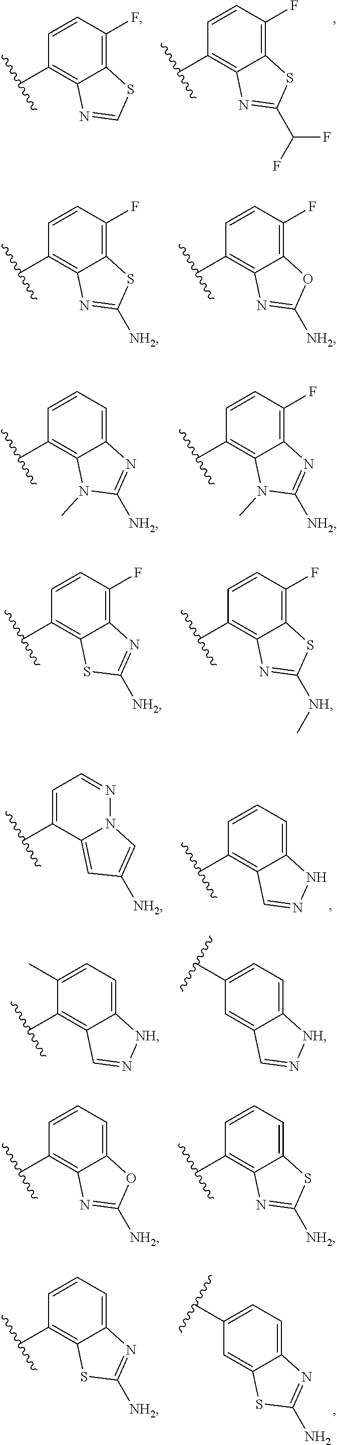

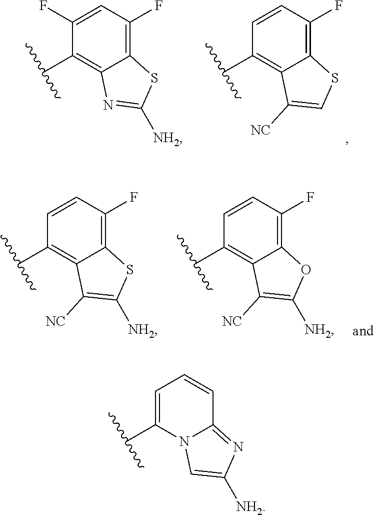 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12]

43. The compound of claim 42, wherein R⁶ is naphthyl substituted with one or more R¹⁵.

44. The compound of any one of claims 1-35, wherein R⁶ is phenyl or monocyclic heteroaryl, wherein the phenyl or heteroaryl are unsubstituted or substituted with one or more R¹⁵.

45. The compound of any one of claims 1-44, wherein R⁴ is H.

46. The compound of any one of claims 1-44, wherein R⁴ is —OR¹².

47. The compound of any one of claims 1-46, wherein R⁵ is selected from H, halogen, —OR¹², —CN, C₁₋₆alkyl, C₂₋₆alkynyl, a 3-6 membered carbocycle, a 5-6 membered heteroaryl, and phenyl, wherein any C₁₋₆alkyl is unsubstituted or substituted with one or more R¹³, and wherein any carbocycle, heteroaryl, and phenyl is unsubstituted or substituted with one or more R¹⁴.

48. The compound of claim 47, wherein R⁵ is H.

49. The compound of claim 47, wherein R⁵ is a halogen.

50. The compound of claim 47, wherein R⁵ is C₁₋₆alkyl, unsubstituted or substituted with one or more R¹³.

51. The compound of claim 47, wherein R⁵ is selected from a 3-6 membered carbocycle, a 5-6 membered heteroaryl, and phenyl, wherein any carbocycle, heteroaryl, and phenyl is unsubstituted or substituted with one or more R¹⁴.

52. The compound of any one of claims 1-51, wherein R⁷ is a halogen.

53. The compound of any one of claims 1-51, wherein R⁷ is —CN.

54. The compound of any one of claims 1-53, wherein:
R² is selected from H and C₁₋₆ alkyl;
R⁵ is selected from halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a bicyclic heteroaryl substituted with one or more R¹⁵;
R⁸ is selected from heterocycle and alkylheterocycle, any of which is unsubstituted or substituted with one or more R¹⁶;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹⁴ is independently selected from halogen, N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹; and
each R²⁰ is independently selected from —OH, —OC₁₋₆alkyl, —CN, —NH₂, —NHC₁₋₆alkyl, and halogen.

55. The compound of any one of claims 1-54, wherein the compound is of Formula IA: 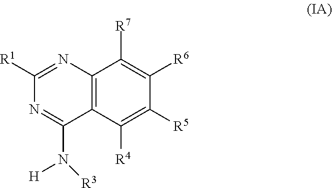
or a pharmaceutically acceptable salt thereof.

56. The compound of claim 55, wherein the compound is of Formula IA1: 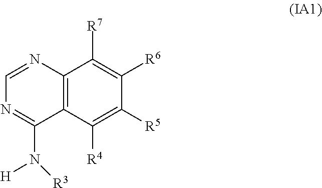
or a pharmaceutically acceptable salt thereof.

57. The compound of claim 55, wherein the compound is of Formula IA2: 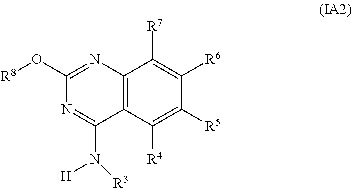
or a pharmaceutically acceptable salt thereof.

58. The compound of any one of claims 1-54, wherein the compound is of Formula IB: 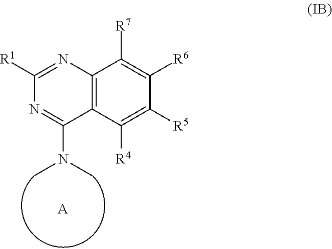
or a pharmaceutically acceptable salt thereof, wherein:
Ring A is a heterocycle that is unsubstituted or substituted with one or more R¹¹.

59. The compound of claim 58, wherein the compound is of Formula IB1: 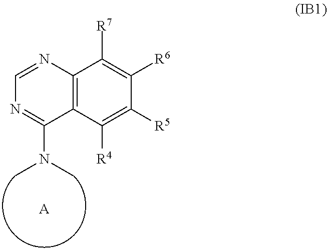
or a pharmaceutically acceptable salt thereof.

60. The compound of claim 58, wherein the compound is of Formula IB2: 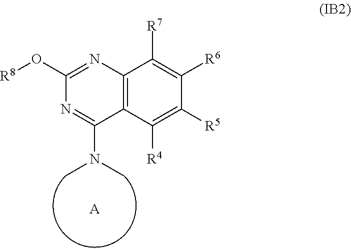
or a pharmaceutically acceptable salt thereof.

61. The compound of any one of claims 1-54, wherein the compound is of Formula IC: 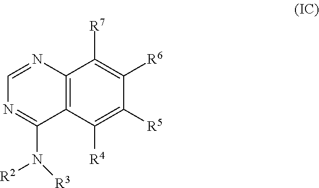
or a pharmaceutically acceptable salt thereof.

62. The compound of any one of claims 1-54, wherein the compound is of Formula ID or ID′: 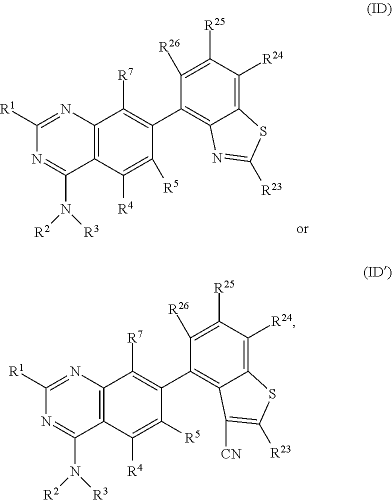
or a pharmaceutically acceptable salt thereof, wherein:
R²³ is selected from —N(R¹²)₂ and C₁₋₆alkyl-N(R¹²)₂; and
R²⁴, R²⁵, and R²⁶ are independently selected from H, halogen, —OR¹², and C₁₋₆alkyl, wherein any C₁₋₆alkyl is unsubstituted or substituted with one or more R¹¹.

63. The compound of any one of claims 1-54, wherein the compound is of Formula IE: 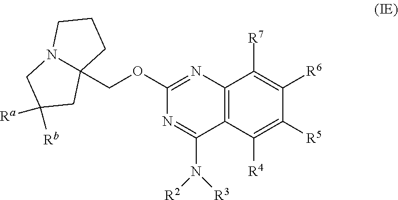
or a pharmaceutically acceptable salt thereof, wherein:
R^{a} and R^{b} are independently selected from halogen, —OR¹², C₁₋₆alkyl, and H, wherein any C₁₋₆alkyl is unsubstituted or is substituted with one or more R¹³.

64. The compound of any one of claims 1-54, wherein the compound is of Formula IF: 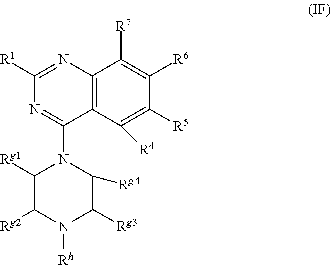
or a pharmaceutically acceptable salt thereof, wherein:
R^{g1}, R^{g2}, R^{g3}, and R^{g4} are each independently selected from H and C₁₋₆alkyl; or (a) R^{g1} and R^{g3} or (b) R^{g2} and R^{g3} join together to form a second ring containing 4-6 members, and any of R^{g1}, R^{g2}, R^{g3}, and R^{g4} that are not part of the second ring are independently selected from H and C₁₋₆alkyl, wherein any C₁₋₆alkyl is unsubstituted or substituted with one or more R¹³; and
R^{h} is selected from H, C₁₋₆alkyl, —C(O)NH₂, and —C(O)C₁₋₆alkylNH₂.

65. A compound according to Formula II: 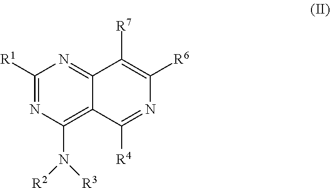
or a pharmaceutically acceptable salt thereof, wherein:
R¹ is selected from —OR⁸, a 4-6 membered heterocycle comprising a nitrogen atom, and H, wherein the heterocycle is unsubstituted or substituted with one or more R¹⁶;
R² is selected from H, a 3-6 membered carbocycle, and C₁₋₆ alkyl, wherein the 3-6 membered carbocycle or the C₁₋₆ alkyl is unsubstituted or substituted with one or more R¹³;
R³ is selected from C₁₋₆ alkyl, a carbocycle, and a heterocycle, wherein any C₁₋₆alkyl is substituted with one or more R⁹, and wherein the carbocycle or heterocycle is unsubstituted or substituted with one or more R¹⁰;
or R² and R³, together with the atom to which they are attached, form a heterocycle that is unsubstituted or substituted with one or more R¹¹;
R⁴ is selected from H, halogen, —OR¹², —CN, C₁₋₆alkyl, C₂₋₆alkenyl, C₂₋₆alkynyl, a 3-6 membered carbocycle, and a 3-6 membered heterocycle, wherein any C₁₋₆alkyl is unsubstituted or substituted with one or more R¹³, and wherein any carbocycle and heterocycle is unsubstituted or substituted with one or more R¹⁴;
R⁶ is a phenyl, monocyclic heteroaryl, bicyclic aryl, or bicyclic heteroaryl, wherein any phenyl, monocyclic heteroaryl, bicyclic aryl, and bicyclic heteroaryl is unsubstituted or substituted with one or more R¹⁵;
R⁷ is selected from halogen, —OR¹², —CN, and H;
R⁸ is selected from heterocycle and alkylheterocycle, any of which is unsubstituted or substituted with one or more R¹⁶, and wherein an alkyl moiety of any alkylheterocycle is selected from C₁₋₆ alkyl;
each R⁹ is independently selected from —N(R¹⁷)₂, —N(R¹⁷)C(O)C₁₋₆alkyl, and —OR¹⁷, wherein any C₁₋₆alkyl is unsubstituted or substituted with one or more R¹⁸;
each R¹⁰ is independently selected from halogen, —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹¹ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR²², —CN, —N(R²²)₂, and halogen;
each R¹⁴ is independently selected from halogen, —OR¹², —CN, —N(R¹²)₂, and C₁₋₆alkyl, wherein any C₁₋₆alkyl is unsubstituted or substituted with one or more R¹³;
each R¹⁵ is independently selected from halogen, —OR¹², —CN, —N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, —OR¹², and 3-6 membered heterocycle, wherein any C₁₋₆alkyl is unsubstituted or substituted with one or more R¹³;
each R¹⁷ is independently selected from C₁₋₆ alkyl and H;
each R¹⁸ is independently selected from —OH, —OC₁₋₆alkyl, —CN, —NH₂, —NHC₁₋₆alkyl, and halogen;
each R¹⁹ is independently selected from C₁₋₆ alkyl, 3-6 membered heterocycle, and H, wherein any C₁₋₆alkyl is unsubstituted or substituted with one or more R²¹;
each R²⁰ is independently selected from —OH, —OC₁₋₆alkyl, —CN, —NH₂, —NHC₁₋₆alkyl, and halogen, and 3-6 membered carbocycle;
each R²¹ is independently selected from —OH, —OC₁₋₆alkyl, —CN, —NH₂, —NHC₁₋₆alkyl, and halogen; and
each R²² is independently selected from C₁₋₆ alkyl, C₂₋₆ alkenyl, and H.

66. The compound of claim 65, wherein R¹ is —OR⁸.

67. The compound of claim 65 or 66, wherein R⁸ is a heterocycle, wherein the heterocycle is unsubstituted or substituted with one or more R¹⁶.

68. The compound of claim 65 or 66, wherein R⁸ is an alkylheterocycle, wherein the alkylheterocycle is unsubstituted or substituted with one or more R¹⁶.

69. The compound of claim 68, wherein the alkylheterocycle is —CH₂(heterocycle), wherein the heterocycle is unsubstituted or substituted with one or more R¹⁶.

70. The compound of any one of claims 65-69, wherein each R¹⁶ is independently selected from halogen, C₁₋₆alkyl, and —OR¹², wherein any C₁₋₆alkyl is unsubstituted or substituted with one or more R¹³.

71. The compound of claim 65, wherein R¹ is a 4-6 membered heterocycle comprising a nitrogen atom, wherein the heterocycle is unsubstituted or substituted with one or more R¹⁶.

72. The compound of claim 65 or 71, wherein each R¹⁶ is independently selected from —N(R¹²)₂, C₁₋₆alkyl, and 3-6 membered heterocycle.

73. The compound of claim 65, wherein R¹ is H.

74. The compound of any one of claims 65-73, wherein R² is H.

75. The compound of any one of claims 65-73, wherein R² is C₁₋₆ alkyl, unsubstituted or substituted with one or more R¹³.

76. The compound of any one of claims 65-73, wherein R² is a 3-6 membered carbocycle.

77. The compound of any one of claim 65-76, wherein R³ is C₁₋₆alkyl substituted with one or more R⁹.

78. The compound of claim 77, wherein R⁹ is —N(R¹⁷)₂.

79. The compound of any one of claims 65-76, wherein R³ is a carbocycle, unsubstituted or substituted with one or more R¹⁰.

80. The compound of any one of claims 65-76, wherein R³ is a heterocycle, unsubstituted or substituted with one or more R¹⁰.

81. The compound of any one of claims 65-76, wherein R² and R³, together with the atom to which they are attached, form a heterocycle that is unsubstituted or substituted with one or more R¹¹.

82. The compound of any one of claims 65-81, wherein R⁶ is a bicyclic aryl or bicyclic heteroaryl, wherein the aryl or heteroaryl are unsubstituted or substituted with one or more R¹⁵.

83. The compound of claim 82, wherein each R¹⁵ is independently selected from halogen, —CN, and —N(R¹²)₂.

84. The compound of any one of claims 65-83, wherein R⁴ is H.

85. The compound of any one of claims 65-84, wherein R⁷ is a halogen.

86. The compound of any one of claim 65-85, wherein:
R² is selected from H and C₁₋₆ alkyl;
R⁶ is a bicyclic heteroaryl substituted with one or more R⁵;
R⁸ is selected from heterocycle and alkylheterocycle, any of which is unsubstituted or substituted with one or more R¹⁶;
each R¹⁰ is independently selected from —N(R¹⁹)₂, —C(O)R¹⁹, —C(O)N(R¹⁹)₂, —C(O)(C₁₋₆alkyl)N(R¹⁹)₂, —(C₁₋₆alkyl)C(O)N(R¹⁹)₂, —C(NR¹⁹)NR¹⁹CN, and C₁₋₆alkyl, wherein any C₁₋₆alkyl is unsubstituted or substituted with one or more R²⁰;
each R¹⁴ is independently selected from halogen, —N(R¹²)₂, and C₁₋₆alkyl, wherein any C₁₋₆alkyl is unsubstituted or substituted with one or more R¹³;
each R¹⁵ is independently selected from halogen, N(R¹²)₂, and C₁₋₆alkyl, wherein any C₁₋₆alkyl is unsubstituted or substituted with one or more R¹³;
each R¹⁶ is independently selected from halogen, —N(R¹²)₂, C₁₋₆alkyl, and —OR¹², wherein any C₁₋₆alkyl is unsubstituted or substituted with one or more R¹³;
each R¹⁹ is independently selected from C₁₋₆ alkyl and H, wherein any C₁₋₆alkyl is unsubstituted or substituted with one or more R²¹; and
each R²⁰ is independently selected from —OH, —OC₁₋₆alkyl, —CN, —NH₂, —NHC₁₋₆alkyl, and halogen.

87. A compound selected from any one of Tables 2-9, or a pharmaceutically acceptable salt thereof.

88. A pharmaceutical composition comprising a compound of any one of claims 1-87, or a pharmaceutically acceptable salt thereof, and a pharmaceutically acceptable excipient.

89. A compound of any one of claims 1-87, or a pharmaceutically acceptable salt thereof, for use as a medicament.

90. A compound of any one of claims 1-87, or a pharmaceutically acceptable salt thereof, for use in the manufacture of a medicament.

91. The compound of claim 89 or 90, wherein the medicament is useful in the treatment of a disease, disorder, or condition ameliorated by the inhibition of KRAS having a G12D mutation.

92. The compound of any one of claims 89-91, wherein the medicament is useful in the treatment of a cancer.

93. A compound of any one of claims 1-87, or a pharmaceutically acceptable salt thereof, for use in the treatment of a disease, disorder, or condition.

94. The compound of claim 93, wherein the disease, disorder, or condition is a cancer.

95. The compound of claim 92 or 94, wherein the cancer is selected from the group consisting of pancreatic cancer, colorectal cancer, and lung cancer.

96. A method, comprising administering a therapeutically effective amount of a compound of any one of claims 1-87, or a pharmaceutically acceptable salt thereof, to a subject in need thereof.

97. The method of claim 96, wherein the subject has a disease, disorder, or condition ameliorated by the inhibition of KRAS having a G12D mutation.

98. The method of claim 96 or 97, wherein the subject has a cancer.

99. The method of claim 98, wherein the subject was previously diagnosed with the cancer.

100. The method of claim 98, wherein the subject has previously undergone a treatment regimen for the cancer.

101. The method of claim 98, wherein the subject has previously entered remission from the cancer.

102. The method of any one of claims 98-101, wherein the cancer is selected from the group consisting of pancreatic cancer, colorectal cancer, and lung cancer.

103. The method of any one of claims 98-102, wherein the compound or pharmaceutically acceptable salt thereof is administered in combination with an additional therapeutic agent.

104. Use of a compound of any one of claims 1-87, or a pharmaceutically acceptable salt thereof, in the manufacture of a medicament.

105. The use of claim 104, wherein the medicament is for treating cancer.

106. The use of claim 105, wherein the cancer is selected from the group consisting of pancreatic cancer, colorectal cancer, and lung cancer.

107. A method, comprising contacting a KRAS protein with a compound of any one of claims 1-87, or a pharmaceutically acceptable salt thereof.

108. The method of claim 107, wherein contacting the KRAS protein with the compound modulates KRAS.

109. The method of claim 107 or 108, wherein the KRAS protein has a G12D mutation.

110. The method of any one of claims 107-109, wherein the KRAS protein is in an active state.

111. The method of any one of claims 107-109, wherein the KRAS protein is in an inactive state.
